SEQUENCE LISTING (352 sequences)

SEQ ID NO: 1 - DNA, 357 bp, synthetic construct
  misc_feature 1..357: note = synthetic
  source 1..357: mol_type = other DNA
caggtgcagctggtggagtctgggggaggcgtggtccagcctggaaggtccctgagactctcctgtgtagcgtctggattcaccttcagtagttatggcattcactgggtccgccaggctccaggcaaggggctggagtgggtggcagttatatgggatgatggaaataatataaactattcagactccgtgaagggccgattcatcatctccagagacaattccaggaagacagtgtatctgcaaatgaacagcctgagaggcgaggacacggctgtttattactgtgcgagagatgcccccatagcaccagtccctgactattggggccagggaaccctggtcaccgtctcctca

SEQ ID NO: 2 - protein, 119 aa, synthetic construct
  REGION 1..119: note = synthetic
QVQLVESGGGVVQPGRSLRLSCVASGFTFSSYGIHWVRQAPGKGLEWVAVIWDDGNNINYSDSVKGRFIISRDNSRKTVYLQMNSLRGEDTAVYYCARDAPIAPVPDYWGQGTLVTVSS

SEQ ID NO: 3 - DNA, 24 bp, synthetic construct
  misc_feature 1..24: note = synthetic
  source 1..24: mol_type = other DNA
ggattcaccttcagtagttatggc

SEQ ID NO: 4 - protein, 8 aa, synthetic construct
  REGION 1..8: note = synthetic
GFTFSSYG

SEQ ID NO: 5 - DNA, 24 bp, synthetic construct
  misc_feature 1..24: note = synthetic
  source 1..24: mol_type = other DNA
atatgggatgatggaaataatata

SEQ ID NO: 6 - protein, 8 aa, synthetic construct
  REGION 1..8: note = synthetic
IWDDGNNI

SEQ ID NO: 7 - DNA, 36 bp, synthetic construct
  misc_feature 1..36: note = synthetic
  source 1..36: mol_type = other DNA
gcgagagatgcccccatagcaccagtccctgactat

SEQ ID NO: 8 - protein, 12 aa, synthetic construct
  REGION 1..12: note = synthetic
ARDAPIAPVPDY

SEQ ID NO: 9 - DNA, 321 bp, synthetic construct
  misc_feature 1..321: note = synthetic
  source 1..321: mol_type = other DNA
gacatccagatgacccagtctccttccaccctgtctgcatctgtaggagacagagtcaccatcacttgccgggccagtcagagtattagtagttggttggcctggtatcagcagaaaccagggaaagcccctaagctcctgatctataaggcgtctagtttagacactggggtcccatcaaggttcagcggcagtggatctgggacagagttcactctcaccatcagcagcctgcagcctgatgattttgcaacttattactgccaacagtataatacttattcgtacacttttggcctggggaccaaactggagatcaaa

SEQ ID NO: 10 - protein, 107 aa, synthetic construct
  REGION 1..107: note = synthetic
DIQMTQSPSTLSASVGDRVTITCRASQSISSWLAWYQQKPGKAPKLLIYKASSLDTGVPSRFSGSGSGTEFTLTISSLQPDDFATYYCQQYNTYSYTFGLGTKLEIK

SEQ ID NO: 11 - DNA, 18 bp, synthetic construct
  misc_feature 1..18: note = synthetic
  source 1..18: mol_type = other DNA
cagagtattagtagttgg

SEQ ID NO: 12 - protein, 6 aa, synthetic construct
  REGION 1..6: note = synthetic
QSISSW

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caacagtataatacttattcgtacact

SEQ ID NO: 16 - protein, 9 aa, synthetic construct
  REGION 1..9: note = synthetic
QQYNTYSYT

SEQ ID NO: 17 - DNA, 357 bp, synthetic construct
  misc_feature 1..357: note = synthetic
  source 1..357: mol_type = other DNA
caggtgcaactggtggagtctgggggaggcttggtcaagcctggagggtccctgagactctcctgtgcagcttctggattcaccttcagtgactactacatgagctggatccgccaggctccagggaaggggctggagtgggtttcatatattagcagtagtggtaataccataaaatatgcagactctatgaagggccgattcaccatctccagggacaacgccaagaaatcactgtttgtggaaatgaacagcctgagagccgaggacacggccgtgtattactgtgcgaggtataaaagttcgtccgactactttgaccactggggccagggaaccctggtcaccgtctcctca

SEQ ID NO: 18 - protein, 119 aa, synthetic construct
  REGION 1..119: note = synthetic
QVQLVESGGGLVKPGGSLRLSCAASGFTFSDYYMSWIRQAPGKGLEWVSYISSSGNTIKYADSMKGRFTISRDNAKKSLFVEMNSLRAEDTAVYYCARYKSSSDYFDHWGQGTLVTVSS

SEQ ID NO: 19 - DNA, 24 bp, synthetic construct
  misc_feature 1..24: note = synthetic
  source 1..24: mol_type = other DNA
ggattcaccttcagtgactactac

SEQ ID NO: 20 - protein, 8 aa, synthetic construct
  REGION 1..8: note = synthetic
GFTFSDYY

SEQ ID NO: 21 - DNA, 24 bp, synthetic construct
  misc_feature 1..24: note = synthetic
  source 1..24: mol_type = other DNA
attagcagtagtggtaataccata

SEQ ID NO: 22 - protein, 8 aa, synthetic construct
  REGION 1..8: note = synthetic
ISSSGNTI

SEQ ID NO: 23 - DNA, 36 bp, synthetic construct
  misc_feature 1..36: note = synthetic
  source 1..36: mol_type = other DNA
gcgaggtataaaagttcgtccgactactttgaccac

SEQ ID NO: 24 - protein, 12 aa, synthetic construct
  REGION 1..12: note = synthetic
ARYKSSSDYFDH

SEQ ID NO: 25 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtgttaggagttacttagcctggtaccaacagaaacctggccaggctcccaggctcctcatctatgatgcatccaacagggccactgccatcccagccaggttcagtggcagtgggtctgggacagacttcactctcaccatcagcagcctagagcctgaagatttagcagtttattactgtcagcagtctggcaactggccgctcactttcggcggagggaccaaggtggagatcaaa

SEQ ID NO: 26 - protein, 107 aa, synthetic construct
  REGION 1..107: note = synthetic
EIVLTQSPATLSLSPGERATLSCRASQSVRSYLAWYQQKPGQAPRLLIYDASNRATAIPARFSGSGSGTDFTLTISSLEPEDLAVYYCQQSGNWPLTFGGGTKVEIK

SEQ ID NO: 27 - DNA, 18 bp, synthetic construct
  misc_feature 1..18: note = synthetic
  source 1..18: mol_type = other DNA
cagagtgttaggagttac

SEQ ID NO: 28 - protein, 6 aa, synthetic construct
  REGION 1..6: note = synthetic
QSVRSY

SEQ ID NO: 29-30 - skipped (2 entries)

SEQ ID NO: 31 - DNA, 27 bp, synthetic construct
  misc_feature 1..27: note = synthetic
  source 1..27: mol_type = other DNA
cagcagtctggcaactggccgctcact

SEQ ID NO: 32 - protein, 9 aa, synthetic construct
  REGION 1..9: note = synthetic
QQSGNWPLT

SEQ ID NO: 33 - DNA, 357 bp, synthetic construct
  misc_feature 1..357: note = synthetic
  source 1..357: mol_type = other DNA
caggtgcagctggtggagtctgggggaggcgtggtccagcctgggaggtccttgagactctcctgtggagcgtctggattcaccttcagtacttatggcatgcactgggtccgccaggctccaggcaaggggctggagtgggtggcagttatctgggatgatggaaataataaatattatgcagactccgtgaagggccgattcaccatctccagagacaattcgaagaacacgctgtatctgcagatgaacagcctgagagccgaggacacggctgtttattactgtgcgagagattcagaggtcgccccagttggggactactggggccagggcaccctggtcaccgtctcctca

SEQ ID NO: 34 - protein, 119 aa, synthetic construct
  REGION 1..119: note = synthetic
QVQLVESGGGVVQPGRSLRLSCGASGFTFSTYGMHWVRQAPGKGLEWVAVIWDDGNNKYYADSVKGRFTISRDNSKNTLYLQMNSLRAEDTAVYYCARDSEVAPVGDYWGQGTLVTVSS

SEQ ID NO: 35 - DNA, 24 bp, synthetic construct
  misc_feature 1..24: note = synthetic
  source 1..24: mol_type = other DNA
ggattcaccttcagtacttatggc

SEQ ID NO: 36 - protein, 8 aa, synthetic construct
  REGION 1..8: note = synthetic
GFTFSTYG

SEQ ID NO: 37 - DNA, 24 bp, synthetic construct
  misc_feature 1..24: note = synthetic
  source 1..24: mol_type = other DNA
atctgggatgatggaaataataaa

SEQ ID NO: 38 - protein, 8 aa, synthetic construct
  REGION 1..8: note = synthetic
IWDDGNNK

SEQ ID NO: 39 - DNA, 36 bp, synthetic construct
  misc_feature 1..36: note = synthetic
  source 1..36: mol_type = other DNA
gcgagagattcagaggtcgccccagttggggactac

SEQ ID NO: 40 - protein, 12 aa, synthetic construct
  REGION 1..12: note = synthetic
ARDSEVAPVGDY

SEQ ID NO: 41 - DNA, 321 bp, synthetic construct
  misc_feature 1..321: note = synthetic
  source 1..321: mol_type = other DNA
gacatccagatgacccagtctccttccaccctgtctgcatctgtaggagacagagtcactatcatttgccgggccagtcagagtattaacaggtggttggcctggtatcagcagaaaccagggaaggcccctaaactcctgatctataaggcgtctagtttagaaagtggggtcccatcaaggttcagcggcagtggatctgggacagaattcactctcaccatcagcagcctgcagcctgatgattttgcagcttattactgccaacagtataatgattattcgtacacttttggccaggggaccaagctggagatcaaa

SEQ ID NO: 42 - protein, 107 aa, synthetic construct
  REGION 1..107: note = synthetic
DIQMTQSPSTLSASVGDRVTIICRASQSINRWLAWYQQKPGKAPKLLIYKASSLESGVPSRFSGSGSGTEFTLTISSLQPDDFAAYYCQQYNDYSYTFGQGTKLEIK

SEQ ID NO: 43 - DNA, 18 bp, synthetic construct
  misc_feature 1..18: note = synthetic
  source 1..18: mol_type = other DNA
cagagtattaacaggtgg

SEQ ID NO: 44 - protein, 6 aa, synthetic construct
  REGION 1..6: note = synthetic
QSINRW

SEQ ID NO: 45-46 - skipped (2 entries)

SEQ ID NO: 47 - DNA, 27 bp, synthetic construct
  misc_feature 1..27: note = synthetic
  source 1..27: mol_type = other DNA
caacagtataatgattattcgtacact

SEQ ID NO: 48 - protein, 9 aa, synthetic construct
  REGION 1..9: note = synthetic
QQYNDYSYT

SEQ ID NO: 49 - DNA, 366 bp, synthetic construct
  misc_feature 1..366: note = synthetic
  source 1..366: mol_type = other DNA
gaggtgcagctggtggagtctgggggagacttggtccagcctggagggtccctgagactctcctgtgcagcctctggattcaccttcagtgaccactatatggactgggtccgccaggctccagggaaggggctggactggattggccgtattagaaacaaagctaacgcttataacacagaatacgccgcgtctgtgagaggcagattcaccatctcaagagatgattcacagaatttactgtatctgcaaatgaacagcctgaaaaccgatgacacggccgtatattattgtgttagagtctggaactacgcctacttcgctatggacgtctggggccaagggaccacggtcaccgtctcctca

SEQ ID NO: 50 - protein, 122 aa, synthetic construct
  REGION 1..122: note = synthetic
EVQLVESGGDLVQPGGSLRLSCAASGFTFSDHYMDWVRQAPGKGLDWIGRIRNKANAYNTEYAASVRGRFTISRDDSQNLLYLQMNSLKTDDTAVYYCVRVWNYAYFAMDVWGQGTTVTVSS

SEQ ID NO: 51 - DNA, 24 bp, synthetic construct
  misc_feature 1..24: note = synthetic
  source 1..24: mol_type = other DNA
ggattcaccttcagtgaccactat

SEQ ID NO: 52 - protein, 8 aa, synthetic construct
  REGION 1..8: note = synthetic
GFTFSDHY

SEQ ID NO: 53 - DNA, 30 bp, synthetic construct
  misc_feature 1..30: note = synthetic
  source 1..30: mol_type = other DNA
attagaaacaaagctaacgcttataacaca

SEQ ID NO: 54 - protein, 10 aa, synthetic construct
  REGION 1..10: note = synthetic
IRNKANAYNT

SEQ ID NO: 55 - DNA, 39 bp, synthetic construct
  misc_feature 1..39: note = synthetic
  source 1..39: mol_type = other DNA
gttagagtctggaactacgcctacttcgctatggacgtc

SEQ ID NO: 56 - protein, 13 aa, synthetic construct
  REGION 1..13: note = synthetic
VRVWNYAYFAMDV

SEQ ID NO: 57 - DNA, 321 bp, synthetic construct
  misc_feature 1..321: note = synthetic
  source 1..321: mol_type = other DNA
gacatccagatgacccagtctccatcctccctatctgcatctgtgggagacagagtcaccatcacttgccggtcaagtcagaacattggaatctttttaaactggtatcaacaaaaaccaggggaagcccctaacctcctgatctccgctgcatccagtttacacagtggggtcccttcaaggttcagtggcagtgggtctgggacagatttcactctcaccatcggcagtctgcagcctgaagattttgcgacttactactgtcaacagacgtacaataccatattcactttcggccctgggaccaaagtggatatcaaa

SEQ ID NO: 58 - protein, 107 aa, synthetic construct
  REGION 1..107: note = synthetic
DIQMTQSPSSLSASVGDRVTITCRSSQNIGIFLNWYQQKPGEAPNLLISAASSLHSGVPSRFSGSGSGTDFTLTIGSLQPEDFATYYCQQTYNTIFTFGPGTKVDIK

SEQ ID NO: 59 - DNA, 18 bp, synthetic construct
  misc_feature 1..18: note = synthetic
  source 1..18: mol_type = other DNA
cagaacattggaatcttt

SEQ ID NO: 60 - protein, 6 aa, synthetic construct
  REGION 1..6: note = synthetic
QNIGIF

SEQ ID NO: 61-62 - skipped (2 entries)

SEQ ID NO: 63 - DNA, 27 bp, synthetic construct
  misc_feature 1..27: note = synthetic
  source 1..27: mol_type = other DNA
caacagacgtacaataccatattcact

SEQ ID NO: 64 - protein, 9 aa, synthetic construct
  REGION 1..9: note = synthetic
QQTYNTIFT

SEQ ID NO: 65 - DNA, 363 bp, synthetic construct
  misc_feature 1..363: note = synthetic
  source 1..363: mol_type = other DNA
gaggtgcagctggtggagtctgggggagacttggtacagcctggggggtccctgagactctcctgtgcagcctctggattcacctttagcagctatgccatgaactgggtccgccagggtccagggaagggactggagtgggtctcagctattagtggtcgtggtgatagtacatactacgcagactccgtgaagggccggctcaccatctccagagacaattccaagaacacgctgtatctgcaaatgaacagcctgagagccgaggacacggccgtatattactgtgtgaaagagggggagcaactcgtctactggtacttcgatctctggggccgtggcaccctggtcaccgtctcctca

SEQ ID NO: 66 - protein, 121 aa, synthetic construct
  REGION 1..121: note = synthetic
EVQLVESGGDLVQPGGSLRLSCAASGFTFSSYAMNWVRQGPGKGLEWVSAISGRGDSTYYADSVKGRLTISRDNSKNTLYLQMNSLRAEDTAVYYCVKEGEQLVYWYFDLWGRGTLVTVSS

SEQ ID NO: 67 - DNA, 24 bp, synthetic construct
  misc_feature 1..24: note = synthetic
  source 1..24: mol_type = other DNA
ggattcacctttagcagctatgcc

SEQ ID NO: 68 - protein, 8 aa, synthetic construct
  REGION 1..8: note = synthetic
GFTFSSYA

SEQ ID NO: 69 - DNA, 24 bp, synthetic construct
  misc_feature 1..24: note = synthetic
  source 1..24: mol_type = other DNA
attagtggtcgtggtgatagtaca

SEQ ID NO: 70 - protein, 8 aa, synthetic construct
  REGION 1..8: note = synthetic
ISGRGDST

SEQ ID NO: 71 - DNA, 42 bp, synthetic construct
  misc_feature 1..42: note = synthetic
  source 1..42: mol_type = other DNA
gtgaaagagggggagcaactcgtctactggtacttcgatctc

SEQ ID NO: 72 - protein, 14 aa, synthetic construct
  REGION 1..14: note = synthetic
VKEGEQLVYWYFDL

SEQ ID NO: 73 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagaccattagcaactttttacattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagatttttcaacttacttctgtcaacagagttacactaccccgctcactttcggcggagggaccaaggtggagatcaaa

SEQ ID NO: 74 - protein, 107 aa, synthetic construct
  REGION 1..107: note = synthetic
DIQMTQSPSSLSASVGDRVTITCRASQTISNFLHWYQQKPGKAPKLLIYAASSLQSGVPSRFSGSGSGTDFTLTISSLQPEDFSTYFCQQSYTTPLTFGGGTKVEIK

SEQ ID NO: 75 - DNA, 18 bp, synthetic construct
  misc_feature 1..18: note = synthetic
  source 1..18: mol_type = other DNA
cagaccattagcaacttt

SEQ ID NO: 76 - protein, 6 aa, synthetic construct
  REGION 1..6: note = synthetic
QTISNF

SEQ ID NO: 77-78 - skipped (2 entries)

SEQ ID NO: 79 - DNA, 27 bp, synthetic construct
  misc_feature 1..27: note = synthetic
  source 1..27: mol_type = other DNA
caacagagttacactaccccgctcact

SEQ ID NO: 80 - protein, 9 aa, synthetic construct
  REGION 1..9: note = synthetic
QQSYTTPLT

SEQ ID NO: 81 - DNA, 363 bp, synthetic construct
  misc_feature 1..363: note = synthetic
  source 1..363: mol_type = other DNA
gaggtgcagctggtggagtctgggggaggcttggtgaggtcgggggggtccctgagactctcctgtgcagcctctggattcacctttaacagatatgccatgacctgggtccgccaggctccagggaaggggctggagtgggtctcagctataagtggtagtggtagcagcacatactacacagactccgtgaaaggccggttcaccatctccagagacaattccaagaattcggtggatctgcaaatgcacagcctgagagtcgaagacacggccatatattattgtgcgagagggactacagtcactacggggtacggtatggacgtctggggccaagggaccacggtcaccgtctcctca

SEQ ID NO: 82 - protein, 121 aa, synthetic construct
  REGION 1..121: note = synthetic
EVQLVESGGGLVRSGGSLRLSCAASGFTFNRYAMTWVRQAPGKGLEWVSAISGSGSSTYYTDSVKGRFTISRDNSKNSVDLQMHSLRVEDTAIYYCARGTTVTTGYGMDVWGQGTTVTVSS

SEQ ID NO: 83 - DNA, 24 bp, synthetic construct
  misc_feature 1..24: note = synthetic
  source 1..24: mol_type = other DNA
ggattcacctttaacagatatgcc

SEQ ID NO: 84 - protein, 8 aa, synthetic construct
  REGION 1..8: note = synthetic
GFTFNRYA

SEQ ID NO: 85 - DNA, 24 bp, synthetic construct
  misc_feature 1..24: note = synthetic
  source 1..24: mol_type = other DNA
ataagtggtagtggtagcagcaca

SEQ ID NO: 86 - protein, 8 aa, synthetic construct
  REGION 1..8: note = synthetic
ISGSGSST

SEQ ID NO: 87 - DNA, 42 bp, synthetic construct
  misc_feature 1..42: note = synthetic
  source 1..42: mol_type = other DNA
gcgagagggactacagtcactacggggtacggtatggacgtc

SEQ ID NO: 88 - protein, 14 aa, synthetic construct
  REGION 1..14: note = synthetic
ARGTTVTTGYGMDV

SEQ ID NO: 89 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccttcacttgccaggcgagtcaggacattaccaattctttaaattggtatcaacagaaacctgggagagcccctaagctcctgatctacgatgcatcgtatttgaaggcaggggtcccatcaagattcagtggaagtggatctgggacagattttactttcaccatcagcagcctgcagcctgaagatattgcaacatattactgtcaacaatatgatgatctcccatacacttttggccaggggaccaagctggagatcaaa

SEQ ID NO: 90 - protein, 107 aa, synthetic construct
  REGION 1..107: note = synthetic
DIQMTQSPSSLSASVGDRVTFTCQASQDITNSLNWYQQKPGRAPKLLIYDASYLKAGVPSRFSGSGSGTDFTFTISSLQPEDIATYYCQQYDDLPYTFGQGTKLEIK

SEQ ID NO: 91 - DNA, 18 bp, synthetic construct
  misc_feature 1..18: note = synthetic
  source 1..18: mol_type = other DNA
caggacattaccaattct

SEQ ID NO: 92 - protein, 6 aa, synthetic construct
  REGION 1..6: note = synthetic
QDITNS

SEQ ID NO: 93-94 - skipped (2 entries)

SEQ ID NO: 95 - DNA, 27 bp, synthetic construct
  misc_feature 1..27: note = synthetic
  source 1..27: mol_type = other DNA
caacaatatgatgatctcccatacact

SEQ ID NO: 96 - protein, 9 aa, synthetic construct
  REGION 1..9: note = synthetic
QQYDDLPYT

SEQ ID NO: 97 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatatttgactactggggccagggaaccctggtcaccgtctcctca

SEQ ID NO: 98 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 99 - DNA, 24 bp, synthetic construct
  misc_feature 1..24: note = synthetic
  source 1..24: mol_type = other DNA
ggtgactccgtcagtagttcctac

SEQ ID NO: 100 - protein, 8 aa, synthetic construct
  REGION 1..8: note = synthetic
GDSVSSSY

SEQ ID NO: 101 - DNA, 21 bp, synthetic construct
  misc_feature 1..21: note = synthetic
  source 1..21: mol_type = other DNA
atctattacagtgggagttcc

SEQ ID NO: 102 - protein, 7 aa, synthetic construct
  REGION 1..7: note = synthetic
IYYSGSS

SEQ ID NO: 103 - DNA, 42 bp, synthetic construct
  misc_feature 1..42: note = synthetic
  source 1..42: mol_type = other DNA
gcgagagaagggaacgtggatacaactatgatatttgactac

SEQ ID NO: 104 - protein, 14 aa, synthetic construct
  REGION 1..14: note = synthetic
AREGNVDTTMIFDY

SEQ ID NO: 105 - DNA, 321 bp, synthetic construct
  misc_feature 1..321: note = synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggacgttcggccaagggaccaaggtggaaatcaaa

SEQ ID NO: 106 - protein, 107 aa, synthetic construct
  REGION 1..107: note = synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 107 - DNA, 18 bp, synthetic construct
  misc_feature 1..18: note = synthetic
  source 1..18: mol_type = other DNA
cagggcattagaaatgat

SEQ ID NO: 108 - protein, 6 aa, synthetic construct
  REGION 1..6: note = synthetic
QGIRND

SEQ ID NO: 109-110 - skipped (2 entries)

SEQ ID NO: 111 - DNA, 27 bp, synthetic construct
  misc_feature 1..27: note = synthetic
  source 1..27: mol_type = other DNA
ctacaagatttcaattacccgtggacg

SEQ ID NO: 112 - protein, 9 aa, synthetic construct
  REGION 1..9: note = synthetic
LQDFNYPWT

SEQ ID NO: 113 - DNA, 321 bp, synthetic construct
  misc_feature 1..321: note = synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtcatcaagatttcaattacccgtggacgttcggccaagggaccaaggtggaaatcaaa

SEQ ID NO: 114 - protein, 107 aa, synthetic construct
  REGION 1..107: note = synthetic
AIQMTQSPSSLSASVGDRVTITCRASQGIRNDLGWYQQKPGKAPKLLIYAASSLQSGVPSRFAGRGSGTDFTLTISSLQPEDFATYYCHQDFNYPWTFGQGTKVEIK

SEQ ID NO: 115 - DNA, 18 bp, synthetic construct
  misc_feature 1..18: note = synthetic
  source 1..18: mol_type = other DNA
cagggcattagaaatgat

SEQ ID NO: 116 - protein, 6 aa, synthetic construct
  REGION 1..6: note = synthetic
QGIRND

SEQ ID NO: 117-118 - skipped (2 entries)

SEQ ID NO: 119 - DNA, 27 bp, synthetic construct
  misc_feature 1..27: note = synthetic
  source 1..27: mol_type = other DNA
catcaagatttcaattacccgtggacg

SEQ ID NO: 120 - protein, 9 aa, synthetic construct
  REGION 1..9: note = synthetic
HQDFNYPWT

SEQ ID NO: 121 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaacataacgtggatacaactatgatatttgactactggggccagggaaccctggtcaccgtctcctca

SEQ ID NO: 122 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREHNVDTTMIFDYWGQGTLVTVSS

SEQ ID NO: 123 - DNA, 24 bp, synthetic construct
  misc_feature 1..24: note = synthetic
  source 1..24: mol_type = other DNA
ggtgactccgtcagtagttcctac

SEQ ID NO: 124 - protein, 8 aa, synthetic construct
  REGION 1..8: note = synthetic
GDSVSSSY

SEQ ID NO: 125 - DNA, 21 bp, synthetic construct
  misc_feature 1..21: note = synthetic
  source 1..21: mol_type = other DNA
atctattacagtgggagttcc

SEQ ID NO: 126 - protein, 7 aa, synthetic construct
  REGION 1..7: note = synthetic
IYYSGSS

SEQ ID NO: 127 - DNA, 42 bp, synthetic construct
  misc_feature 1..42: note = synthetic
  source 1..42: mol_type = other DNA
gcgagagaacataacgtggatacaactatgatatttgactac

SEQ ID NO: 128 - protein, 14 aa, synthetic construct
  REGION 1..14: note = synthetic
AREHNVDTTMIFDY

SEQ ID NO: 129 - DNA, 321 bp, synthetic construct
  misc_feature 1..321: note = synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggcacttcggccaagggaccaaggtggaaatcaaa

SEQ ID NO: 130 - protein, 107 aa, synthetic construct
  REGION 1..107: note = synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWHFGQGTKVEIK

SEQ ID NO: 131 - DNA, 18 bp, synthetic construct
  misc_feature 1..18: note = synthetic
  source 1..18: mol_type = other DNA
cagggcattagaaatgat

SEQ ID NO: 132 - protein, 6 aa, synthetic construct
  REGION 1..6: note = synthetic
QGIRND

SEQ ID NO: 133-134 - skipped (2 entries)

SEQ ID NO: 135 - DNA, 27 bp, synthetic construct
  misc_feature 1..27: note = synthetic
  source 1..27: mol_type = other DNA
ctacaagatttcaattacccgtggcac

SEQ ID NO: 136 - protein, 9 aa, synthetic construct
  REGION 1..9: note = synthetic
LQDFNYPWH

SEQ ID NO: 137 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatacatgactactggggccagggaaccctggtcaccgtctcctca

SEQ ID NO: 138 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIHDYWGQGTLVTVSS

SEQ ID NO: 139 - DNA, 24 bp, synthetic construct
  misc_feature 1..24: note = synthetic
  source 1..24: mol_type = other DNA
ggtgactccgtcagtagttcctac

SEQ ID NO: 140 - protein, 8 aa, synthetic construct
  REGION 1..8: note = synthetic
GDSVSSSY

SEQ ID NO: 141 - DNA, 21 bp, synthetic construct
  misc_feature 1..21: note = synthetic
  source 1..21: mol_type = other DNA
atctattacagtgggagttcc

SEQ ID NO: 142 - protein, 7 aa, synthetic construct
  REGION 1..7: note = synthetic
IYYSGSS

SEQ ID NO: 143 - DNA, 42 bp, synthetic construct
  misc_feature 1..42: note = synthetic
  source 1..42: mol_type = other DNA
gcgagagaagggaacgtggatacaactatgatacatgactac

SEQ ID NO: 144 - protein, 14 aa, synthetic construct
  REGION 1..14: note = synthetic
AREGNVDTTMIHDY

SEQ ID NO: 145 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatcacactatgatatttgactactggggccagggaaccctggtcaccgtctcctca

SEQ ID NO: 146 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDHTMIFDYWGQGTLVTVSS

SEQ ID NO: 147 - DNA, 24 bp, synthetic construct
  misc_feature 1..24: note = synthetic
  source 1..24: mol_type = other DNA
ggtgactccgtcagtagttcctac

SEQ ID NO: 148 - protein, 8 aa, synthetic construct
  REGION 1..8: note = synthetic
GDSVSSSY

SEQ ID NO: 149 - DNA, 21 bp, synthetic construct
  misc_feature 1..21: note = synthetic
  source 1..21: mol_type = other DNA
atctattacagtgggagttcc

SEQ ID NO: 150 - protein, 7 aa, synthetic construct
  REGION 1..7: note = synthetic
IYYSGSS

SEQ ID NO: 151 - DNA, 42 bp, synthetic construct
  misc_feature 1..42: note = synthetic
  source 1..42: mol_type = other DNA
gcgagagaagggaacgtggatcacactatgatatttgactac

SEQ ID NO: 152 - protein, 14 aa, synthetic construct
  REGION 1..14: note = synthetic
AREGNVDHTMIFDY

SEQ ID NO: 153 - DNA, 360 bp, synthetic construct
  misc_feature 1..360: note = synthetic
  source 1..360: mol_type = other DNA
caggtgcagctggtggagtctgggggaggcttggtcaagcctggagggtccctgagactctcctgtgcagcctctggattcaccttcagtgactcctacatgtcctggatccgtcaggctccagggaagggactagagtggatttcatacattggtagtagtggtaataccttttactacgcagactctgtgaagggccggttcaccatttccagagacaacgccaacaatttactgtatctgcaaatgaccagcctgagagccgaggacacggccgtgtattactgtgcgagagaagaaggcgatttttggagtgccgttgactcctggggccagggaaccctggtcaccgtctcctca

SEQ ID NO: 154 - protein, 120 aa, synthetic construct
  REGION 1..120: note = synthetic
QVQLVESGGGLVKPGGSLRLSCAASGFTFSDSYMSWIRQAPGKGLEWISYIGSSGNTFYYADSVKGRFTISRDNANNLLYLQMTSLRAEDTAVYYCAREEGDFWSAVDSWGQGTLVTVSS

SEQ ID NO: 155 - DNA, 24 bp, synthetic construct
  misc_feature 1..24: note = synthetic
  source 1..24: mol_type = other DNA
ggattcaccttcagtgactcctac

SEQ ID NO: 156 - protein, 8 aa, synthetic construct
  REGION 1..8: note = synthetic
GFTFSDSY

SEQ ID NO: 157 - DNA, 24 bp, synthetic construct
  misc_feature 1..24: note = synthetic
  source 1..24: mol_type = other DNA
attggtagtagtggtaataccttt

SEQ ID NO: 158 - protein, 8 aa, synthetic construct
  REGION 1..8: note = synthetic
IGSSGNTF

SEQ ID NO: 159 - DNA, 39 bp, synthetic construct
  misc_feature 1..39: note = synthetic
  source 1..39: mol_type = other DNA
gcgagagaagaaggcgatttttggagtgccgttgactcc

SEQ ID NO: 160 - protein, 13 aa, synthetic construct
  REGION 1..13: note = synthetic
AREEGDFWSAVDS

SEQ ID NO: 161 - DNA, 321 bp, synthetic construct
  misc_feature 1..321: note = synthetic
  source 1..321: mol_type = other DNA
gacatccagttgacccagtctccatccttcctgtctgcatctgtaggagacagagtcaccatcacttgctgggccagtcagggcattagcagttatttagcctggtatcagcaaaaaccaggtaaagcccctaaactcctgatccatactgcatccactttgcaaagtggggtcccatcaaggttcagcggcagtggatctgggacagaattcactctcacaatcagcaacctgcagcctgaagattttgcaacttattactgtcaacagcttaatagttacccattcactttcggccctgggaccaaagtggatatcaaa

SEQ ID NO: 162 - protein, 107 aa, synthetic construct
  REGION 1..107: note = synthetic
DIQLTQSPSFLSASVGDRVTITCWASQGISSYLAWYQQKPGKAPKLLIHTASTLQSGVPSRFSGSGSGTEFTLTISNLQPEDFATYYCQQLNSYPFTFGPGTKVDIK

SEQ ID NO: 163 - DNA, 18 bp, synthetic construct
  misc_feature 1..18: note = synthetic
  source 1..18: mol_type = other DNA
cagggcattagcagttat

SEQ ID NO: 164 - protein, 6 aa, synthetic construct
  REGION 1..6: note = synthetic
QGISSY

SEQ ID NO: 165-166 - skipped (2 entries)

SEQ ID NO: 167 - DNA, 27 bp, synthetic construct
  misc_feature 1..27: note = synthetic
  source 1..27: mol_type = other DNA
caacagcttaatagttacccattcact

SEQ ID NO: 168 - protein, 9 aa, synthetic construct
  REGION 1..9: note = synthetic
QQLNSYPFT

SEQ ID NO: 169 - DNA, 366 bp, synthetic construct
  misc_feature 1..366: note = synthetic
  source 1..366: mol_type = other DNA
caggtgcagctggtggagtctgggggaggcgtggtccagcctggggggtccctgagactctcctgtgcagcctctggattcaccttcggtggccatgccatgcactgggtccgccaggctccaggcaaggggctggagtggctggcagttatatcatctgatggcagtaataaacagtatgcagattctgtgaagggccgattcaccatctccagggacaatcccaagaacacgctgtatctgcaaatgaacagtctgagagttggggacacggctatttattactgtgcgaaagaggtggcacctcgttattattattacggtctggacgtctggggccaagggaccacggtcaccgtctcctca

SEQ ID NO: 170 - protein, 122 aa, synthetic construct
  REGION 1..122: note = synthetic
QVQLVESGGGVVQPGGSLRLSCAASGFTFGGHAMHWVRQAPGKGLEWLAVISSDGSNKQYADSVKGRFTISRDNPKNTLYLQMNSLRVGDTAIYYCAKEVAPRYYYYGLDVWGQGTTVTVSS

SEQ ID NO: 171 - DNA, 24 bp, synthetic construct
  misc_feature 1..24: note = synthetic
  source 1..24: mol_type = other DNA
ggattcaccttcggtggccatgcc

SEQ ID NO: 172 - protein, 8 aa, synthetic construct
  REGION 1..8: note = synthetic
GFTFGGHA

SEQ ID NO: 173 - DNA, 24 bp, synthetic construct
  misc_feature 1..24: note = synthetic
  source 1..24: mol_type = other DNA
atatcatctgatggcagtaataaa

SEQ ID NO: 174 - protein, 8 aa, synthetic construct
  REGION 1..8: note = synthetic
ISSDGSNK

SEQ ID NO: 175 - DNA, 45 bp, synthetic construct
  misc_feature 1..45: note = synthetic
  source 1..45: mol_type = other DNA
gcgaaagaggtggcacctcgttattattattacggtctggacgtc

SEQ ID NO: 176 - protein, 15 aa, synthetic construct
  REGION 1..15: note = synthetic
AKEVAPRYYYYGLDV

SEQ ID NO: 177 - DNA, 318 bp, synthetic construct
  misc_feature 1..318: note = synthetic
  source 1..318: mol_type = other DNA
gacatccagatgacccagtctccatcctccctgtctgcatctgtgggagacagagtcaccatcacttgccgggcgagtcaggacattagcaattttttagcctggtatcagcagaaaccagggaaggttcctaaactcctgatctatactgcatccactttacaatcaggggtcccatctcggttcagtggcagtggatctgggacagatttcactctcaccgtcagcagcctacagcctgaagatgttgcaacttattactgtcaaaagtatgccggcgccctcactttcggccctgggaccaaagtggatatcaaa

SEQ ID NO: 178 - protein, 106 aa, synthetic construct
  REGION 1..106: note = synthetic
DIQMTQSPSSLSASVGDRVTITCRASQDISNFLAWYQQKPGKVPKLLIYTASTLQSGVPSRFSGSGSGTDFTLTVSSLQPEDVATYYCQKYAGALTFGPGTKVDIK

SEQ ID NO: 179 - DNA, 18 bp, synthetic construct
  misc_feature 1..18: note = synthetic
  source 1..18: mol_type = other DNA
caggacattagcaatttt

SEQ ID NO: 180 - protein, 6 aa, synthetic construct
  REGION 1..6: note = synthetic
QDISNF

SEQ ID NO: 181-182 - skipped (2 entries)

SEQ ID NO: 183 - DNA, 24 bp, synthetic construct
  misc_feature 1..24: note = synthetic
  source 1..24: mol_type = other DNA
caaaagtatgccggcgccctcact

SEQ ID NO: 184 - protein, 8 aa, synthetic construct
  REGION 1..8: note = synthetic
QKYAGALT

SEQ ID NO: 185 - DNA, 357 bp, synthetic construct
  misc_feature 1..357: note = synthetic
  source 1..357: mol_type = other DNA
gaggtgcagctggtggagtctgggggaggcttggcacagcctggggggtccctgagactctcctgtgcagcctctggattcacgtttagaagctatgccatgagctgggtccgccaggctccagggaaggggccggagtgggtctcaggtataggtggtaatggtgttaccacatactacgcagactccgtgaagggccggttcaccatctccagagacaattccaagaacacgctgtttctgcaaatgaatagcctgagagccgaggacacggccgtatattattgtgtgcaggggggtttaggtggttattttacaggctactggggccagggaaccctggtcaccgtctcctca

SEQ ID NO: 186 - protein, 119 aa, synthetic construct
  REGION 1..119: note = synthetic
EVQLVESGGGLAQPGGSLRLSCAASGFTFRSYAMSWVRQAPGKGPEWVSGIGGNGVTTYYADSVKGRFTISRDNSKNTLFLQMNSLRAEDTAVYYCVQGGLGGYFTGYWGQGTLVTVSS

SEQ ID NO: 187 - DNA, 24 bp, synthetic construct
  misc_feature 1..24: note = synthetic
  source 1..24: mol_type = other DNA
ggattcacgtttagaagctatgcc

SEQ ID NO: 188 - protein, 8 aa, synthetic construct
  REGION 1..8: note = synthetic
GFTFRSYA

SEQ ID NO: 189 - DNA, 24 bp, synthetic construct
  misc_feature 1..24: note = synthetic
  source 1..24: mol_type = other DNA
ataggtggtaatggtgttaccaca

SEQ ID NO: 190 - protein, 8 aa, synthetic construct
  REGION 1..8: note = synthetic
IGGNGVTT

SEQ ID NO: 191 - DNA, 36 bp, synthetic construct
  misc_feature 1..36: note = synthetic
  source 1..36: mol_type = other DNA
gtgcaggggggtttaggtggttattttacaggctac

SEQ ID NO: 192 - protein, 12 aa, synthetic construct
  REGION 1..12: note = synthetic
VQGGLGGYFTGY

SEQ ID NO: 193 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagtattagtacctatttaaattggtatcagcagaatccagggaaagcccctaaactcctgatctttgatgcatccagtttgcaaagtggggtcccatcaaggttcagtggcagtggatctgggacagatttcactctcaccatcagaggtctgcaacctgaagattttgcaacttactactgtcaacagagttacagtgccccgctcactttcggcggagggaccaaggtggagatcaaa

SEQ ID NO: 194 - protein, 107 aa, synthetic construct
  REGION 1..107: note = synthetic
DIQMTQSPSSLSASVGDRVTITCRASQSISTYLNWYQQNPGKAPKLLIFDASSLQSGVPSRFSGSGSGTDFTLTIRGLQPEDFATYYCQQSYSAPLTFGGGTKVEIK

SEQ ID NO: 195 - DNA, 18 bp, synthetic construct
  misc_feature 1..18: note = synthetic
  source 1..18: mol_type = other DNA
cagagtattagtacctat

SEQ ID NO: 196 - protein, 6 aa, synthetic construct
  REGION 1..6: note = synthetic
QSISTY

SEQ ID NO: 197-198 - skipped (2 entries)

SEQ ID NO: 199 - DNA, 27 bp, synthetic construct
  misc_feature 1..27: note = synthetic
  source 1..27: mol_type = other DNA
caacagagttacagtgccccgctcact

SEQ ID NO: 200 - protein, 9 aa, synthetic construct
  REGION 1..9: note = synthetic
QQSYSAPLT

SEQ ID NO: 201 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcgtctggattcaccttcagtggttatggcatgcactgggtccgccaggctccaggcaaggggctggagtgggtggcacttatatggcttgatggaagtaatgactactatgcagactccgtgaagggccgattcaccatctccagagacaattccaagaacacgttatatctgcaaatgaacagactgagagccgaggacacggctgtgtattactgtgcgagagatggcccggttgctgctatacccgactactggggccagggaaccctggtcaccgtctcctca

SEQ ID NO: 202 - protein, 119 aa, synthetic construct
  REGION 1..119: note = synthetic
QVQLVESGGGVVQPGRSLRLSCAASGFTFSGYGMHWVRQAPGKGLEWVALIWLDGSNDYYADSVKGRFTISRDNSKNTLYLQMNRLRAEDTAVYYCARDGPVAAIPDYWGQGTLVTVSS

SEQ ID NO: 203 - DNA, 24 bp, synthetic construct
  misc_feature 1..24: note = synthetic
  source 1..24: mol_type = other DNA
ggattcaccttcagtggttatggc

SEQ ID NO: 204 - protein, 8 aa, synthetic construct
  REGION 1..8: note = synthetic
GFTFSGYG

SEQ ID NO: 205 - DNA, 24 bp, synthetic construct
  misc_feature 1..24: note = synthetic
  source 1..24: mol_type = other DNA
atatggcttgatggaagtaatgac

SEQ ID NO: 206 - protein, 8 aa, synthetic construct
  REGION 1..8: note = synthetic
IWLDGSND

SEQ ID NO: 207 - DNA, 36 bp, synthetic construct
  misc_feature 1..36: note = synthetic
  source 1..36: mol_type = other DNA
gcgagagatggcccggttgctgctatacccgactac

SEQ ID NO: 208 - protein, 12 aa, synthetic construct
  REGION 1..12: note = synthetic
ARDGPVAAIPDY

SEQ ID NO: 209 - DNA, 321 bp, synthetic construct
  misc_feature 1..321: note = synthetic
  source 1..321: mol_type = other DNA
gacatccagatgacccagtctccttccaccctgtctgcatctgtaggagacagagtcaccatcacttgccgggccagtcagagtattagtaggtggttggcctggtatcagctgaaaccagggaaagcccctaagctcctgatctataaggcgtctagtttagaaagtggggtcccatcaaggttcagcggcagtggatctgggacagacttcactctcaccatcagcagcctgcaacctgatgattttgcaacttattactgccaacagtataatacttattcgtacacttttggccaggggaccaagctggagatcaaa

SEQ ID NO: 210 - protein, 107 aa, synthetic construct
  REGION 1..107: note = synthetic
DIQMTQSPSTLSASVGDRVTITCRASQSISRWLAWYQLKPGKAPKLLIYKASSLESGVPSRFSGSGSGTDFTLTISSLQPDDFATYYCQQYNTYSYTFGQGTKLEIK

SEQ ID NO: 211 - DNA, 18 bp, synthetic construct
  misc_feature 1..18: note = synthetic
  source 1..18: mol_type = other DNA
cagagtattagtaggtgg

SEQ ID NO: 212 - protein, 6 aa, synthetic construct
  REGION 1..6: note = synthetic
QSISRW

SEQ ID NO: 213-214 - skipped (2 entries)

SEQ ID NO: 215 - DNA, 27 bp, synthetic construct
  misc_feature 1..27: note = synthetic
  source 1..27: mol_type = other DNA
caacagtataatacttattcgtacact

SEQ ID NO: 216 - protein, 9 aa, synthetic construct
  REGION 1..9: note = synthetic
QQYNTYSYT

SEQ ID NO: 217 - DNA, 363 bp, synthetic construct
  misc_feature 1..363: note = synthetic
  source 1..363: mol_type = other DNA
gaggtgcagctggtggagtctgggggaggtgtggtacggcctggggggtccctgagactctcctgtgcagcctctggattcacctttgatgaatatggcatgacttgggtccgccaagttccagggaaggggctggagtgggtctctggtattacttggaatggtggtttcacagattatacagactctgtgaagggccgattcaccagctccagagacaacgccaagaactccctgtatctgcaaatgaacagtctgagagccgaggacacggccttgtattactgtgcgagagatggatatagcagctcgtggggggcttatgatatatggggccaagggacaatggtcaccgtctcttca

SEQ ID NO: 218 - protein, 121 aa, synthetic construct
  REGION 1..121: note = synthetic
EVQLVESGGGVVRPGGSLRLSCAASGFTFDEYGMTWVRQVPGKGLEWVSGITWNGGFTDYTDSVKGRFTSSRDNAKNSLYLQMNSLRAEDTALYYCARDGYSSSWGAYDIWGQGTMVTVSS

SEQ ID NO: 219 - DNA, 24 bp, synthetic construct
  misc_feature 1..24: note = synthetic
  source 1..24: mol_type = other DNA
ggattcacctttgatgaatatggc

SEQ ID NO: 220 - protein, 8 aa, synthetic construct
  REGION 1..8: note = synthetic
GFTFDEYG

SEQ ID NO: 221 - DNA, 24 bp, synthetic construct
  misc_feature 1..24: note = synthetic
  source 1..24: mol_type = other DNA
attacttggaatggtggtttcaca

SEQ ID NO: 222 - protein, 8 aa, synthetic construct
  REGION 1..8: note = synthetic
ITWNGGFT

SEQ ID NO: 223 - DNA, 42 bp, synthetic construct
  misc_feature 1..42: note = synthetic
  source 1..42: mol_type = other DNA
gcgagagatggatatagcagctcgtggggggcttatgatata

SEQ ID NO: 224 - protein, 14 aa, synthetic construct
  REGION 1..14: note = synthetic
ARDGYSSSWGAYDI

SEQ ID NO: 225 - DNA, 321 bp, synthetic construct
  misc_feature 1..321: note = synthetic
  source 1..321: mol_type = other DNA
gacatccagatgacccagtctccatcatccctgtctgcatctgtgggagacagagtcaccatcacttgccgggcaagtcagagcattagcacctatttaaattggtatcagcagaaaccagggaaagcccctaagctcctgatctatgctgcatccagtttgcaaagtggggtcccattaaggttcagtggcagtggatctgggactgatttcactctcaccatcagcagtctgcaacctgaagattttgcaagttatttctgtcaacagagttacagtaccccgtacacttttggccaggggaccaagctggagatcaaa

SEQ ID NO: 226 - protein, 107 aa, synthetic construct
  REGION 1..107: note = synthetic
DIQMTQSPSSLSASVGDRVTITCRASQSISTYLNWYQQKPGKAPKLLIYAASSLQSGVPLRFSGSGSGTDFTLTISSLQPEDFASYFCQQSYSTPYTFGQGTKLEIK

SEQ ID NO: 227 - DNA, 18 bp, synthetic construct
  misc_feature 1..18: note = synthetic
  source 1..18: mol_type = other DNA
cagagcattagcacctat

SEQ ID NO: 228 - protein, 6 aa, synthetic construct
  REGION 1..6: note = synthetic
QSISTY

SEQ ID NO: 229-230 - skipped (2 entries)

SEQ ID NO: 231 - DNA, 27 bp, synthetic construct
  misc_feature 1..27: note = synthetic
  source 1..27: mol_type = other DNA
caacagagttacagtaccccgtacact

SEQ ID NO: 232 - protein, 9 aa, synthetic construct
  REGION 1..9: note = synthetic
QQSYSTPYT

SEQ ID NO: 233 - DNA, 360 bp, synthetic construct
  misc_feature 1..360: note = synthetic
  source 1..360: mol_type = other DNA
gaagtgcagctggtggagtctgggggaggcgtggtacagcctggggggtccctgagactctcctgtgcagcctctggattcacctttaatgattatgccatgcactgggtccgtcaagctccagggaagggtctggagtgggtctctcttattagtggagatggtggtaacacatactatgcagactctgtgaagggccgactcaccatctccagagacaacagcaaaaactccctgtatctgcaaatgaacagtctgagaacagaggacaccgccttatattactgtgcaaaagataagggctggaacttcggttacttcgatctctggggccgtggcaccctggtcactgtctcctca

SEQ ID NO: 234 - protein, 120 aa, synthetic construct
  REGION 1..120: note = synthetic
EVQLVESGGGVVQPGGSLRLSCAASGFTFNDYAMHWVRQAPGKGLEWVSLISGDGGNTYYADSVKGRLTISRDNSKNSLYLQMNSLRTEDTALYYCAKDKGWNFGYFDLWGRGTLVTVSS

SEQ ID NO: 235 - DNA, 24 bp, synthetic construct
  misc_feature 1..24: note = synthetic
  source 1..24: mol_type = other DNA
ggattcacctttaatgattatgcc

SEQ ID NO: 236 - protein, 8 aa, synthetic construct
  REGION 1..8: note = synthetic
GFTFNDYA

SEQ ID NO: 237 - DNA, 24 bp, synthetic construct
  misc_feature 1..24: note = synthetic
  source 1..24: mol_type = other DNA
attagtggagatggtggtaacaca

SEQ ID NO: 238 - protein, 8 aa, synthetic construct
  REGION 1..8: note = synthetic
ISGDGGNT

SEQ ID NO: 239 - DNA, 39 bp, synthetic construct
  misc_feature 1..39: note = synthetic
  source 1..39: mol_type = other DNA
gcaaaagataagggctggaacttcggttacttcgatctc

SEQ ID NO: 240 - protein, 13 aa, synthetic construct
  REGION 1..13: note = synthetic
AKDKGWNFGYFDL

SEQ ID NO: 241 - DNA, 327 bp, synthetic construct
  misc_feature 1..327: note = synthetic
  source 1..327: mol_type = other DNA
gacatccagatgacccagtctccatcctccctgtctacatctgtgggagacagagtcaccatcacttgccgggcaagtcagaacattgacacctatttaaattggtatcagcagaaaccagggaaagcccctaaactcctgatctatgatgcatccagtttacaaagtggggtcccatcacggttcagtggcagcggatctgggacagatttcactctcaccatcaccagtctgcaacctgaagattttgccacttactactgtcaacagaatgacaatattcttcaccctctcactttcggcggagggaccaaggtggagatcaaa

SEQ ID NO: 242 - protein, 109 aa, synthetic construct
  REGION 1..109: note = synthetic
DIQMTQSPSSLSTSVGDRVTITCRASQNIDTYLNWYQQKPGKAPKLLIYDASSLQSGVPSRFSGSGSGTDFTLTITSLQPEDFATYYCQQNDNILHPLTFGGGTKVEIK

SEQ ID NO: 243 - DNA, 18 bp, synthetic construct
  misc_feature 1..18: note = synthetic
  source 1..18: mol_type = other DNA
cagaacattgacacctat

SEQ ID NO: 244 - protein, 6 aa, synthetic construct
  REGION 1..6: note = synthetic
QNIDTY

SEQ ID NO: 245-246 - skipped (2 entries)

SEQ ID NO: 247 - DNA, 33 bp, synthetic construct
  misc_feature 1..33: note = synthetic
  source 1..33: mol_type = other DNA
caacagaatgacaatattcttcaccctctcact

SEQ ID NO: 248 - protein, 11 aa, synthetic construct
  REGION 1..11: note = synthetic
QQNDNILHPLT

SEQ ID NO: 249 - DNA, 369 bp, synthetic construct
  misc_feature 1..369: note = synthetic
  source 1..369: mol_type = other DNA
gaggtgcagctggtggagtctgggggaggcttggtccaaccgggggggtccctgagactctcctgtgcagcctctggattccactctaatagatattggatggactgggtccgccaggctccagggaaggggctggagtgggtggccaacataaagcaagatggaagtgaggaaaactatgtggactctgtgaagggccgattcaccatctccagagacaacgccaagaactcactttatctgcaaatgaacagcctgagagccgaggacacggctgtgtattactgtgcgagagatcgaagcacctcgtgggtcccttactggttcttcgatctctggggccgtggcaccctggtcactgtctcctca

SEQ ID NO: 250 - protein, 123 aa, synthetic construct
  REGION 1..123: note = synthetic
EVQLVESGGGLVQPGGSLRLSCAASGFHSNRYWMDWVRQAPGKGLEWVANIKQDGSEENYVDSVKGRFTISRDNAKNSLYLQMNSLRAEDTAVYYCARDRSTSWVPYWFFDLWGRGTLVTVSS

SEQ ID NO: 251 - DNA, 24 bp, synthetic construct
  misc_feature 1..24: note = synthetic
  source 1..24: mol_type = other DNA
ggattccactctaatagatattgg

SEQ ID NO: 252 - protein, 8 aa, synthetic construct
  REGION 1..8: note = synthetic
GFHSNRYW

SEQ ID NO: 253 - DNA, 24 bp, synthetic construct
  misc_feature 1..24: note = synthetic
  source 1..24: mol_type = other DNA
ataaagcaagatggaagtgaggaa

SEQ ID NO: 254 - protein, 8 aa, synthetic construct
  REGION 1..8: note = synthetic
IKQDGSEE

SEQ ID NO: 255 - DNA, 48 bp, synthetic construct
  misc_feature 1..48: note = synthetic
  source 1..48: mol_type = other DNA
gcgagagatcgaagcacctcgtgggtcccttactggttcttcgatctc

SEQ ID NO: 256 - protein, 16 aa, synthetic construct
  REGION 1..16: note = synthetic
ARDRSTSWVPYWFFDL

SEQ ID NO: 257 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 258 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 259 - DNA, 18 bp, synthetic construct
  misc_feature 1..18: note = synthetic
  source 1..18: mol_type = other DNA
cagagcattagcagctat

SEQ ID NO: 260 - protein, 6 aa, synthetic construct
  REGION 1..6: note = synthetic
QSISSY

SEQ ID NO: 261-262 - skipped (2 entries)

SEQ ID NO: 263 - DNA, 30 bp, synthetic construct
  misc_feature 1..30: note = synthetic
  source 1..30: mol_type = other DNA
caacagagttacagtacccctccgatcacc

SEQ ID NO: 264 - protein, 10 aa, synthetic construct
  REGION 1..10: note = synthetic
QQSYSTPPIT

SEQ ID NO: 265 - DNA, 360 bp, synthetic construct
  misc_feature 1..360: note = synthetic
  source 1..360: mol_type = other DNA
gaagtgcagctggtggagtctgggggaggcgtggtacagcggggggagtccctgagactctcctgttcagcctctgacttcatctttaaagattatgccatgtactgggtccgtcaaattccagggaagggtctagagtggatctctcttattagtggtgatggtgacactacatggtatggagactctgtgaagggccgattcaccatctccagagacaacaacgaaaactccctctttctgcaaatgaacgatctgagaactgaggacaccgccatgtactactgtgcaagagatatggggtggaacttctttcagttgcaatactggggccagggaaccctggtcaccgtctcctca

SEQ ID NO: 266 - protein, 120 aa, synthetic construct
  REGION 1..120: note = synthetic
EVQLVESGGGVVQRGESLRLSCSASDFIFKDYAMYWVRQIPGKGLEWISLISGDGDTTWYGDSVKGRFTISRDNNENSLFLQMNDLRTEDTAMYYCARDMGWNFFQLQYWGQGTLVTVSS

SEQ ID NO: 267 - DNA, 24 bp, synthetic construct
  misc_feature 1..24: note = synthetic
  source 1..24: mol_type = other DNA
gacttcatctttaaagattatgcc

SEQ ID NO: 268 - protein, 8 aa, synthetic construct
  REGION 1..8: note = synthetic
DFIFKDYA

SEQ ID NO: 269 - DNA, 24 bp, synthetic construct
  misc_feature 1..24: note = synthetic
  source 1..24: mol_type = other DNA
attagtggtgatggtgacactaca

SEQ ID NO: 270 - protein, 8 aa, synthetic construct
  REGION 1..8: note = synthetic
ISGDGDTT

SEQ ID NO: 271 - DNA, 39 bp, synthetic construct
  misc_feature 1..39: note = synthetic
  source 1..39: mol_type = other DNA
gcaagagatatggggtggaacttctttcagttgcaatac

SEQ ID NO: 272 - protein, 13 aa, synthetic construct
  REGION 1..13: note = synthetic
ARDMGWNFFQLQY

SEQ ID NO: 273 - DNA, 361 bp, synthetic construct
  misc_feature 1..361: note = synthetic
  source 1..361: mol_type = other DNA
dcaggtgcagctgcaggagtcgggccccgcactggtgaagccttcacagaccctgtccctcacctgcactgtctctggtggctccatcatcagaggtagtacctactggagttgggtccgccaattcccagggaagggcctggagtggattggatacagttattacagtgggaccgcctactataatccgtccctcgagagtcgagctaccatttctgtagacacgtctaagaaccagttctccctgaacctgaagtctgtgacggccgcggacacggccgtgtattattgtacaagagaaataggagtggctggtctctttgacatctggggccagggaaccctggtcaccgtctcctca

SEQ ID NO: 274 - protein, 120 aa, synthetic construct
  REGION 1..120: note = synthetic
QVQLQESGPALVKPSQTLSLTCTVSGGSIIRGSTYWSWVRQFPGKGLEWIGYSYYSGTAYYNPSLESRATISVDTSKNQFSLNLKSVTAADTAVYYCTREIGVAGLFDIWGQGTLVTVSS

SEQ ID NO: 275 - DNA, 30 bp, synthetic construct
  misc_feature 1..30: note = synthetic
  source 1..30: mol_type = other DNA
ggtggctccatcatcagaggtagtacctac

SEQ ID NO: 276 - protein, 10 aa, synthetic construct
  REGION 1..10: note = synthetic
GGSIIRGSTY

SEQ ID NO: 277 - DNA, 21 bp, synthetic construct
  misc_feature 1..21: note = synthetic
  source 1..21: mol_type = other DNA
agttattacagtgggaccgcc

SEQ ID NO: 278 - protein, 7 aa, synthetic construct
  REGION 1..7: note = synthetic
SYYSGTA

SEQ ID NO: 279 - DNA, 36 bp, synthetic construct
  misc_feature 1..36: note = synthetic
  source 1..36: mol_type = other DNA
acaagagaaataggagtggctggtctctttgacatc

SEQ ID NO: 280 - protein, 12 aa, synthetic construct
  REGION 1..12: note = synthetic
TREIGVAGLFDI

SEQ ID NO: 281 - DNA, 324 bp, synthetic construct
  misc_feature 1..324: note = synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 282 - protein, 108 aa, synthetic construct
  REGION 1..108: note = synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 283 - DNA, 36 bp, synthetic construct
  misc_feature 1..36: note = synthetic
  source 1..36: mol_type = other DNA
agagcaagtcagtcagtctctagctcttatctcgcc

SEQ ID NO: 284 - protein, 12 aa, synthetic construct
  REGION 1..12: note = synthetic
RASQSVSSSYLA

SEQ ID NO: 285 - DNA, 21 bp, synthetic construct
  misc_feature 1..21: note = synthetic
  source 1..21: mol_type = other DNA
ggggcaagttccagggccacc

SEQ ID NO: 286 - protein, 7 aa, synthetic construct
  REGION 1..7: note = synthetic
GASSRAT

SEQ ID NO: 287 - DNA, 27 bp, synthetic construct
  misc_feature 1..27: note = synthetic
  source 1..27: mol_type = other DNA
caacagtacggaagcagcccgtggacg

SEQ ID NO: 288 - protein, 9 aa, synthetic construct
  REGION 1..9: note = synthetic
QQYGSSPWT

SEQ ID NO: 289 - DNA, 357 bp, synthetic construct
  misc_feature 1..357: note = synthetic
  source 1..357: mol_type = other DNA
caggagcagttggtgcagtctggggctgaggtgaagaagcctggggcctcagtgaaggtctcctgtaaggcttctggatacaccttcaccggctactatatacattgggtgcgacaggcccctggactagggcttgaatggatgggatggatcaaccctaacagtggtggcacaaaatatgcacagaagtttcagggcagggtcaccatgaccagggacacgtccatcaatacagcctacatggagctgaaaagactgaaatctgacgactcggccgtatattactgtgcgagagacgcccctccccatgatgtttttgatatctggggccaagggacattggtcaccgtctcttca

SEQ ID NO: 290 - protein, 119 aa, synthetic construct
  REGION 1..119: note = synthetic
QEQLVQSGAEVKKPGASVKVSCKASGYTFTGYYIHWVRQAPGLGLEWMGWINPNSGGTKYAQKFQGRVTMTRDTSINTAYMELKRLKSDDSAVYYCARDAPPHDVFDIWGQGTLVTVSS

SEQ ID NO: 291 - DNA, 24 bp, synthetic construct
  misc_feature 1..24: note = synthetic
  source 1..24: mol_type = other DNA
ggatacaccttcaccggctactat

SEQ ID NO: 292 - protein, 8 aa, synthetic construct
  REGION 1..8: note = synthetic
GYTFTGYY

SEQ ID NO: 293 - DNA, 24 bp, synthetic construct
  misc_feature 1..24: note = synthetic
  source 1..24: mol_type = other DNA
atcaaccctaacagtggtggcaca

SEQ ID NO: 294 - protein, 8 aa, synthetic construct
  REGION 1..8: note = synthetic
INPNSGGT

SEQ ID NO: 295 - DNA, 36 bp, synthetic construct
  misc_feature 1..36: note = synthetic
  source 1..36: mol_type = other DNA
gcgagagacgcccctccccatgatgtttttgatatc

SEQ ID NO: 296 - protein, 12 aa, synthetic construct
  REGION 1..12: note = synthetic
ARDAPPHDVFDI

SEQ ID NO: 297 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaattggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaa

SEQ ID NO: 298 - protein, 107 aa, synthetic construct
  REGION 1..107: note = synthetic
DIQMTQSPSSLSASVGDRVTITCRASQGIRNDLGWYQQKPGKAPKRLIYAASSLQIGVPSRFSGSGSGTEFTLTISSLQPEDFATYYCLQHNSYPLTFGGGTKVEIK

SEQ ID NO: 299 - DNA, 18 bp, synthetic construct
  misc_feature 1..18: note = synthetic
  source 1..18: mol_type = other DNA
cagggcattagaaatgat

SEQ ID NO: 300 - protein, 6 aa, synthetic construct
  REGION 1..6: note = synthetic
QGIRND

SEQ ID NO: 301-302 - skipped (2 entries)

SEQ ID NO: 303 - DNA, 27 bp, synthetic construct
  misc_feature 1..27: note = synthetic
  source 1..27: mol_type = other DNA
ctacagcataatagttacccgctcact

SEQ ID NO: 304 - protein, 9 aa, synthetic construct
  REGION 1..9: note = synthetic
LQHNSYPLT

SEQ ID NO: 305 - DNA, 357 bp, synthetic construct
  misc_feature 1..357: note = synthetic
  source 1..357: mol_type = other DNA
caggtgcagctgcaagagtcgggcccaggactggtgaagccttcacagaccctgtccctcacctgcactgtctctggtggctccatcagtagtggtgcttaccactggagctggatccgccagcacccagggaagggcctagagtggattggatacatctattacaatggggacacctactataatccgtccctcaagagtcgcgttaccatttcagtggacacgtctaagaaccaattcttcctgaaggtgacctctgtgactgccgcggacacggccatgtattactgtgcgggagaaaagcagctgactgcttttgatatctggggccaagggacattggtcaccgtctcttca

SEQ ID NO: 306 - protein, 119 aa, synthetic construct
  REGION 1..119: note = synthetic
QVQLQESGPGLVKPSQTLSLTCTVSGGSISSGAYHWSWIRQHPGKGLEWIGYIYYNGDTYYNPSLKSRVTISVDTSKNQFFLKVTSVTAADTAMYYCAGEKQLTAFDIWGQGTLVTVSS

SEQ ID NO: 307 - DNA, 30 bp, synthetic construct
  misc_feature 1..30: note = synthetic
  source 1..30: mol_type = other DNA
ggtggctccatcagtagtggtgcttaccac

SEQ ID NO: 308 - protein, 10 aa, synthetic construct
  REGION 1..10: note = synthetic
GGSISSGAYH

SEQ ID NO: 309 - DNA, 21 bp, synthetic construct
  misc_feature 1..21: note = synthetic
  source 1..21: mol_type = other DNA
atctattacaatggggacacc

SEQ ID NO: 310 - protein, 7 aa, synthetic construct
  REGION 1..7: note = synthetic
IYYNGDT

SEQ ID NO: 311 - DNA, 33 bp, synthetic construct
  misc_feature 1..33: note = synthetic
  source 1..33: mol_type = other DNA
gcgggagaaaagcagctgactgcttttgatatc

SEQ ID NO: 312 - protein, 11 aa, synthetic construct
  REGION 1..11: note = synthetic
AGEKQLTAFDI

SEQ ID NO: 313 - DNA, 321 bp, synthetic construct
  misc_feature 1..321: note = synthetic
  source 1..321: mol_type = other DNA
gtcatccagatgacccagtctccatcctccctgtctgcatctgttggagacagagtcaccataacttgccgggcgagtcaggacattaataattttttaaattggtatcaacagaaattagggaaagcccctaaactcctgatctccgatgcatccaatttgcagacaggagtcccgtcaaggttcagtggaagtggatctgggacagattttactttcaccatcagcagcctgcagcctgaagatattgctgcatattactgtcaacaatatgatcatttcccgtatacttttggccaggggaccagactggagaacaat

SEQ ID NO: 314 - protein, 107 aa, synthetic construct
  REGION 1..107: note = synthetic
VIQMTQSPSSLSASVGDRVTITCRASQDINNFLNWYQQKLGKAPKLLISDASNLQTGVPSRFSGSGSGTDFTFTISSLQPEDIAAYYCQQYDHFPYTFGQGTRLENN

SEQ ID NO: 315 - DNA, 18 bp, synthetic construct
  misc_feature 1..18: note = synthetic
  source 1..18: mol_type = other DNA
caggacattaataatttt

SEQ ID NO: 316 - protein, 6 aa, synthetic construct
  REGION 1..6: note = synthetic
QDINNF

SEQ ID NO: 317-318 - skipped (2 entries)

SEQ ID NO: 319 - DNA, 27 bp, synthetic construct
  misc_feature 1..27: note = synthetic
  source 1..27: mol_type = other DNA
caacaatatgatcatttcccgtatact

SEQ ID NO: 320 - protein, 9 aa, synthetic construct
  REGION 1..9: note = synthetic
QQYDHFPYT

SEQ ID NO: 321 - DNA, 357 bp, synthetic construct
  misc_feature 1..357: note = synthetic
  source 1..357: mol_type = other DNA
gaggtgcagttggtggagtctgggggaggtgtggttcggcctggggggtccctgagactctcctgtgcagcctctggattcacctttgatgattatggcatgacctgggtccgccaagctccagggaaggggctggagtgggtctctggtattaattggaatggcgatagcacagagtattcagactctgtgaagggccgattcaccatctccagagacaacgccaagaactccctgtatctgcaaatgaacagtctgagagccgaggacacggccttctatcactgtgcgagagagaataactggaacttctactttgactactggggccagggaaccctggtcaccgtctcctca

SEQ ID NO: 322 - protein, 119 aa, synthetic construct
  REGION 1..119: note = synthetic
EVQLVESGGGVVRPGGSLRLSCAASGFTFDDYGMTWVRQAPGKGLEWVSGINWNGDSTEYSDSVKGRFTISRDNAKNSLYLQMNSLRAEDTAFYHCARENNWNFYFDYWGQGTLVTVSS

SEQ ID NO: 323 - DNA, 24 bp, synthetic construct
  misc_feature 1..24: note = synthetic
  source 1..24: mol_type = other DNA
ggattcacctttgatgattatggc

SEQ ID NO: 324 - protein, 8 aa, synthetic construct
  REGION 1..8: note = synthetic
GFTFDDYG

SEQ ID NO: 325 - DNA, 24 bp, synthetic construct
  misc_feature 1..24: note = synthetic
  source 1..24: mol_type = other DNA
attaattggaatggcgatagcaca

SEQ ID NO: 326 - protein, 8 aa, synthetic construct
  REGION 1..8: note = synthetic
INWNGDST

SEQ ID NO: 327 - DNA, 36 bp, synthetic construct
  misc_feature 1..36: note = synthetic
  source 1..36: mol_type = other DNA
gcgagagagaataactggaacttctactttgactac

SEQ ID NO: 328 - protein, 12 aa, synthetic construct
  REGION 1..12: note = synthetic
ARENNWNFYFDY

SEQ ID NO: 329 - DNA, 321 bp, synthetic construct
  misc_feature 1..321: note = synthetic
  source 1..321: mol_type = other DNA
gaaatagtgatgacgcagtctccagccaccctgtctgtgtctcgaggggaaagagccaccctctcctgtagggccagtcagagtgttagcagcaacttagcctggtaccagcagaaacttggccaggctcccaggctcctcatctatggtgcatccaccagggccactggtatcccagccaggttcagtggcagtgggtctgggacagagttcactctcaccatcagcagcctgcagtctgaagattttgcagtttattattgtcagcagtataataactggccgtggacgttcggccaagggaccaaggtggaaatcaaa

SEQ ID NO: 330 - protein, 107 aa, synthetic construct
  REGION 1..107: note = synthetic
EIVMTQSPATLSVSRGERATLSCRASQSVSSNLAWYQQKLGQAPRLLIYGASTRATGIPARFSGSGSGTEFTLTISSLQSEDFAVYYCQQYNNWPWTFGQGTKVEIK

SEQ ID NO: 331 - DNA, 18 bp, synthetic construct
  misc_feature 1..18: note = synthetic
  source 1..18: mol_type = other DNA
cagagtgttagcagcaac

SEQ ID NO: 332 - protein, 6 aa, synthetic construct
  REGION 1..6: note = synthetic
QSVSSN

SEQ ID NO: 333-334 - skipped (2 entries)

SEQ ID NO: 335 - DNA, 27 bp, synthetic construct
  misc_feature 1..27: note = synthetic
  source 1..27: mol_type = other DNA
cagcagtataataactggccgtggacg

SEQ ID NO: 336 - protein, 9 aa, synthetic construct
  REGION 1..9: note = synthetic
QQYNNWPWT

SEQ ID NO: 337 - DNA, 351 bp, synthetic construct
  misc_feature 1..351: note = synthetic
  source 1..351: mol_type = other DNA
caggtccacctggtacagtctggggctgaggtgaagaagcctggggcctcagtgaaggtctcctgcaaggtttccggaaacaccctcactgaattatccatgcactgggtgcgacaggctcctggaaaagggcttgagtggatgggaggttttgatcctgaagatggtgacacaatctactcacagaagttccagggcagagtcaccttgaccgaggacacatctacagacacagcctacatggagctgagcagcctgagatctgaggacacggccgtgtattactgttcaacagtggggggacctacctctgactgctggggccagggaaccctggtcaccgtctcctca

SEQ ID NO: 338 - protein, 117 aa, synthetic construct
  REGION 1..117: note = synthetic
QVHLVQSGAEVKKPGASVKVSCKVSGNTLTELSMHWVRQAPGKGLEWMGGFDPEDGDTIYSQKFQGRVTLTEDTSTDTAYMELSSLRSEDTAVYYCSTVGGPTSDCWGQGTLVTVSS

SEQ ID NO: 339 - DNA, 24 bp, synthetic construct
  misc_feature 1..24: note = synthetic
  source 1..24: mol_type = other DNA
ggaaacaccctcactgaattatcc

SEQ ID NO: 340 - protein, 8 aa, synthetic construct
  REGION 1..8: note = synthetic
GNTLTELS

SEQ ID NO: 341 - DNA, 24 bp, synthetic construct
  misc_feature 1..24: note = synthetic
  source 1..24: mol_type = other DNA
tttgatcctgaagatggtgacaca

SEQ ID NO: 342 - protein, 8 aa, synthetic construct
  REGION 1..8: note = synthetic
FDPEDGDT

SEQ ID NO: 343 - DNA, 30 bp, synthetic construct
  misc_feature 1..30: note = synthetic
  source 1..30: mol_type = other DNA
tcaacagtggggggacctacctctgactgc

SEQ ID NO: 344 - protein, 10 aa, synthetic construct
  REGION 1..10: note = synthetic
STVGGPTSDC

SEQ ID NO: 345 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaaggtcctgatcttcgatgcatccaatttagaaccaggggtcccatcaaggttcagtggaagtggatctgggacagattttactttcaccatcatcagcctgcagcctgaagatattgcaacatattactgtcaacaatatgataatctcccgatcaccttcggccaggggacacgactggacattaaa

SEQ ID NO: 346 - protein, 107 aa, synthetic construct
  REGION 1..107: note = synthetic
DIQMTQSPSSLSASVGDRVTITCQASQDISNYLNWYQQKPGKAPKVLIFDASNLEPGVPSRFSGSGSGTDFTFTIISLQPEDIATYYCQQYDNLPITFGQGTRLDIK

SEQ ID NO: 347 - DNA, 18 bp, synthetic construct
  misc_feature 1..18: note = synthetic
  source 1..18: mol_type = other DNA
caggacattagcaactat

SEQ ID NO: 348 - protein, 6 aa, synthetic construct
  REGION 1..6: note = synthetic
QDISNY

SEQ ID NO: 349-350 - skipped (2 entries)

SEQ ID NO: 351 - DNA, 27 bp, synthetic construct
  misc_feature 1..27: note = synthetic
  source 1..27: mol_type = other DNA
caacaatatgataatctcccgatcacc

SEQ ID NO: 352 - protein, 9 aa, synthetic construct
  REGION 1..9: note = synthetic
QQYDNLPIT

(19) United States
(12) Patent Application Publication
(10) Pub. No.: US 2024/0175027 A1
(43) Pub. Date: May 30, 2024

(54) ANTI-C5 ANTIBODY/C5 IRNA CO-FORMULATIONS AND COMBINATION THERAPIES

(71) Applicant: Regeneron Pharmaceuticals, Inc., Tarrytown, NY (US)

(72) Inventors: Mary Kleppe, Milford, CT (US); Hunter Hong-Chun Chen, New York, NY (US); Sarah Ingram, New York, NY (US); Xiaolin Tang, Old Tappan, NJ (US); George D. Yancopoulos, Yorktown Heights, NY (US); Umesh Chaudhari, Kendall Park, NJ (US); Jonathan Weyne, Chappaqua, NY (US); Lorah Perlee, Westport, CT (US); Jeffrey Trevenen, Highland Lakes, NJ (US); Kuan-Ju Lin, Stamford, CT (US)

(21) Appl. No.: 18/496,632

(22) Filed: Oct. 27, 2023

Related U.S. Application Data
(60) Provisional application No. 63/505,011, filed on May 30, 2023.
(60) Provisional application No. 63/498,112, filed on Apr. 25, 2023.
(60) Provisional application No. 63/495,767, filed on Apr. 12, 2023.
(60) Provisional application No. 63/386,787, filed on Dec. 9, 2022.
(60) Provisional application No. 63/385,909, filed on Dec. 2, 2022.
(60) Provisional application No. 63/383,442, filed on Nov. 11, 2022.
(60) Provisional application No. 63/382,264, filed on Nov. 3, 2022.
(60) Provisional application No. 63/382,087, filed on Nov. 2, 2022.
(60) Provisional application No. 63/381,450, filed on Oct. 28, 2022.

(30) Foreign Application Priority Data
Oct. 26, 2023 (TW) 112141079

(51) Int. Cl. C12N 15/113; A61K 9/00; A61K 45/06; A61K 47/18; A61K 47/22; A61K 47/26; A61K 47/54; A61P 7/00; C07K 16/28; A61K 39/00

(52) U.S. Cl. CPC C12N 15/113; A61K 9/0019; A61K 45/06; A61K 47/183; A61K 47/22; A61K 47/26; A61K 47/549; A61P 7/00; C07K 16/2896; A61K 2039/505; A61K 2039/54; A61K 2039/545; C07K 2317/565; C12N 2310/14

(57) ABSTRACT

The present disclosure provides a co-formulation that includes an antibody that binds specifically to C5 and a C5 iRNA which is a glycoconjugate that includes a ligand having terminal N-Acetylgalactosamine (GalNAc) residues and/or N-acetylglucosamine (GlcNAc) residues. Methods for reducing degradation of glycoconjugate RNA by beta-hexosaminidase enzyme are also provided. The present disclosure also includes methods for treating or preventing a C5-associated disease or disorder by administering one or more doses of an anti-C5 antibody or antigen-binding fragment thereof in combination with one or more doses of a C5 iRNA; preferably wherein the anti-C5 antibody or fragment and the C5 iRNA are in a co-formulation. The present disclosure also includes dosing regimens for treating C5-associated disease or disorder with a combination of anti-C5 antibody and C5 iRNA in subjects that either are treatment naïve or are switching from a previous C5 inhibitor therapy.

### CROSS-REFERENCE TO RELATED APPLICATIONS

The present application claims the benefit of U.S. Provisional Patent Appl. No. 63/381,450 filed Oct. 28, 2022, U.S. Provisional Patent Appl. No. 63/382,087 filed Nov. 2, 2022, U.S. Provisional Patent Appl. No. 63/382,264 filed Nov. 3, 2022, U.S. Provisional Patent Appl. No. 63/383,442 filed Nov. 11, 2022, U.S. Provisional Patent Appl. No. 63/385,909 filed Dec. 2, 2022, U.S. Provisional Patent Appl. No. 63/386,787 filed Dec. 9, 2022, U.S. Provisional Patent Appl. No. 63/495,767 filed Apr. 12, 2023, U.S. Provisional Patent Appl. No. 63/498,112 filed Apr. 25, 2023, U.S. Provisional Patent Appl. No. 63/505,011 filed May 30, 2023, and Taiwanese Patent Appl. No. 112141079 filed Oct. 26, 2023, each of which is herein incorporated by reference in its entirety.

### SEQUENCE LISTING

The sequence listing of the present application is submitted electronically as an ASCII formatted sequence listing with a file name “11282seqlist”, creation date of Oct. 28, 2022, and a size of 112 Kb. This sequence listing submitted is part of the specification and is herein incorporated by reference in its entirety.

### FIELD

The field of the present disclosure relates to co-formulations and combination therapies that include an RNA and an antibody or antigen-binding fragment thereof along with methods for stabilizing RNA in a composition including beta-hexosaminidase.

### BACKGROUND

Complement component C5 is target for several rare diseases, including paroxysmal nocturnal hemoglobinuria (PNH), atypical hemolytic uremic syndrome, neuromyelitis optica, and generalized myasthenia gravis. Uncontrolled complement activation in PNH patients, for example, results in the primarily clinical manifestation of chronic hemolysis, as well as an increased risk of thromboembolism, leading to target organ damage and death.

Complement 5 is a validated target in the treatment of complement-mediated diseases such as generalized myasthenia gravis (gMG) as was established by the approval of Eculizumab (Ecu) for the treatment of patients with gMG. Anti-C5 antibody, Pozelimab, monotherapy has been shown to be effective in blocking C5 activity in another disease (paroxysmal nocturnal hemoglobinuria [PNH]) that is highly sensitive to complement mediated effects.

A great degree of complement inhibition is necessary to provide rapid and pronounced disease suppression and attain complete and uninterrupted inhibition of C5 throughout the entire C5-inhibitor dosing interval. Current C5-inhibitor monotherapies have not achieved a sufficient level of inhibition.

Therapies targeting C5 for PNH, such as Eculizumab and Ravulizumab (Soliris and Ultomiris, Alexion Pharmaceuticals), have demonstrated efficacy. However, in rare instances, Eculizumab and Ravulizumab are ineffective due to polymorphic variation in the gene encoding C5 such that the C5 protein is not bound by Eculizumab or Ravulizumab (Nishimura et al., Genetic variants in C5 and poor response to Eculizumab. N Engl J Med 2014; 370(7):632-639). In addition, treatment is burdensome as the drugs are generally administered chronically by IV infusion Q2W or Q8W, respectively, to maintain efficacy. Moreover, it has been reported that as many as 20% of patients with PNH on Eculizumab treatment at the labeled maintenance dose (900 mg Q2W IV) require significant increases in dose or dose frequency due to breakthrough hemolysis secondary to incomplete inhibition of C5 (Peffault de Latour et al., Assessing complement blockade in patients with paroxysmal nocturnal hemoglobinuria receiving Eculizumab. Blood 2015; 125(5):775-783) (Hillmen et al., Long-term safety and efficacy of sustained Eculizumab treatment in patients with paroxysmal nocturnal haemoglobinuria. Br J Haematol 2013; 162(1):62-73). While the regulatory approval of Ravulizumab has provided an agent with an IV dosing frequency of Q8W, patients still experience some hemolytic breakthrough (Lee et al., Ravulizumab (ALXN1210) vs Eculizumab in adult patients with PNH naive to complement inhibitors: the 301 study. Blood 2019; 133(6):530-539).

A regimen of 30 mg/kg IV loading dose followed by 800 mg SC weekly was effective in a phase 2 study in complement treatment naïve patients with PNH (R3918-PNH-1852) at reducing serum LDH to <1.5×ULN in all patients and <1.0 ULN in most patients. However, the regimen represents relatively high doses for a biologic agent.

The requirement for such high anti-C5 mAb doses is driven by the need for 100% inhibition which is achieved with complete target engagement (Peffault de Latour, 2015) and C5 levels are high; and in order to achieve 100% inhibition on a population basis, inter- and intra-patient variability of C5 concentrations and instances of enhanced complement activation (which may occur with intercurrent illness) must be considered.

Cemdisiran is a synthetic small interfering ribonucleic acid (siRNA) targeting C5 messenger ribonucleic acid (mRNA) that is covalently linked to a triantennary N-acetylgalactosamine (GalNAc) ligand. Cemdisiran is designed to suppress liver production of C5 protein, when administered via SC injection. C5 is encoded by a single gene and is expressed and secreted predominantly by hepatocytes. Through the ribonucleic acid (RNA) interference pathway, Cemdisiran leads to the degradation of C5 mRNA by RNases, thereby reducing C5 protein production, leading to reduced levels of circulating C5 protein. Cemdisiran monotherapy has been found to be insufficiently effective as a monotherapy treatment for PNH. Badri et al., Clin Pharmacokinet. 2021; 60(3):365-78-Epub 2020/10/14.

Combining Cemdisiran with recombinant antibodies in a co-formulation that can conveniently be administered in a common injection raises the risk of contaminants from the antibody degrading the Cemdisiran molecule.

Moreover, treating patients suffering from a condition such as PNH raises the likelihood that a substantial portion of such patients will either be currently receiving another anti-C5 antibody or have recently received such an antibody and, thus, have detectable blood concentrations thereof. Experiments suggest that antibodies having the sequence of Eculizumab and Pozelimab, in combination, were able to form high molecular weight heteromeric complexes with C5, thus posing the risk of formation of such complexes in vivo when both antibodies are present in the circulation.

Results of a previous clinical study reported adverse reactions (e.g., serum sickness-like reactions, skin rash) upon switching from one C5 mAb to another, specifically, upon switching from eculizumab to crovalimab (SKY59/RO7112689/RG6107), a therapeutic C5 antibody that binds a different epitope than Eculizumab. These reactions were attributed to the formation of DTD immune complexes comprising C5 and the two C5 antibodies (Röth et al., The complement C5 inhibitor crovalimab in paroxysmal nocturnal hemoglobinuria. Blood 2020; 135(12):912-920; Röth et al., The SMART Anti-hC5 Antibody (SKY59/RO7112689) Shows Good Safety and Efficacy in Patients with Paroxysmal Nocturnal Hemoglobinuria (PNH). Blood 2018a; 132(Suppl 1):535; United States Patent Publication US2009/0220508). The size of such immune complexes has been related to the occurrence of adverse events. For example, in a study where anti-drug-antibody (ADA)-positive patients were injected with infliximab, an antibody with specificity for a target unrelated to the complement system, a severe infusion reaction was observed when immune complexes larger than 1000 kDa (>6 antibodies) were detected for 1 patient, but not when only smaller immune complexes were detected (<1000 kDa) in 2 patients (van der Laken et al., Imaging and serum analysis of immune complex formation of radiolabelled infliximab and anti-infliximab in responders and non-responders to therapy for rheumatoid arthritis. Ann Rheum Dis 2007; 66(2):253-256), suggesting that large DTD immune complexes are more likely to be associated with adverse events. Furthermore, small DTD immune complexes are expected to be clinically insignificant based on extrapolation from other autoimmune disease states such as systemic lupus erythematous, whereby small immune complexes are inefficient in complement activation and interactions with Fc gamma receptor and do not deposit in tissues (Wener et al., Immune Complexes in Systemic Lupus Erythematosus (Chapter 19). Systemic Lupus Erythematosus. Academic Press; 2010).

Reducing the likelihood of such adverse events is difficult. In the COMMODORE-1 clinical trial, had two arms wherein patients were treated with the anti-C5 antibody, Crovalimab or Eculizumab during a 24 week primary treatment period. After the primary treatment period, patients in the Eculizumab had the option to switch to Crovalimab treatment. Sixteen percent of patients switching from Eculizumab to Crovalimab experienced type 3 hypersensitivity (T3H) reactions. T3H reactions and injection-related reactions were not applicable to the eculizumab arm as they were related to large DTD complex formation and subcutaneous administration, respectively, which are exclusive to the crovalimab arm. Scheinberg et al., Phase III Randomized, Multicenter, Open-Label Commodore 1 Trial: Comparison of Crovalimab vs Eculizumab in Complement Inhibitor-Experienced Patients With Paroxysmal Nocturnal Hemoglobinuria, European Hematology Association, Frankfurt, Germany; Virtual (Hybrid) 9 Jun. 2023.

### SUMMARY

The present invention includes a co-formulation including a C5 iRNA which is conjugated to a ligand that comprises one or more terminal amino sugars, such as N-Acetylgalactosamine (GalNAc) and/or N-acetylglucosamine (GlcNAc) residues; an antibody or antigen-binding fragment thereof that binds specifically to C5 (anti-C5) which is isolated from a mammalian host cell; having a pH of greater than or less than about 6 (e.g., about 6.5); and a pharmaceutically acceptable carrier. For example, in an embodiment of the invention, the co-formulation includes a C5 iRNA; an antibody or antigen-binding fragment thereof that binds specifically to C5; a buffer (e.g., histidine-based buffer, a citrate-based buffer, a phosphate-based buffer and/or an acetate-based buffer, for example, at a concentration of about 10-35, 35-45, 20-50, 20, 25, 30, 35, 40, 45 or 50 mM); a stabilizer (e.g., a polyol, a sugar, trehalose, sorbitol, mannitol, taurine, propane sulfonic acid, L-proline, sucrose, glycerol, threitol, maltitol, polyethylene glycol (PEG), and/or PEG3350; for example, at a concentration of about 0.8-3.6, 0.8, 0.9, 1.0, 1.25, 1.50, 2.0, 2.25, 2.50, 2.75, 3.00, 3.1, 3.2, 3.3, 3.4, 3.5 or 3.6% (w/v)); a viscosity reducer, and a non-ionic surfactant (e.g., a polyoxyethylene glycol alkyl ether; glucoside alkyl ether; polyoxyethylene glycol octylphenol ether; polyoxyethylene glycol alkylphenol ether; glycerol alkyl ester; polyoxyethylene glycol sorbitan alkyl ester; sorbitan alkyl ester; block copolymer of polypropylene glycol; block copolymer of polyethylene glycol; a polysorbate, octaethylene glycol monododecyl ether; pentaethylene glycol monododecyl ether; polyoxypropylene glycol alkyl ether; decyl glucoside, lauryl glucoside, octyl glucoside; triton X-100; nonoxynol-9; glyceryl laurate; cocamide MEA, cocamide DEA, dodecyldimethylamine oxide; poloxamer; polyethoxylated tallow amine (POEA); polysorbate-20 (PS20) and/or polysorbate-80 (PS80); for example, at a concentration of about 0.025, 0.05, 0.075, 0.1, 0.125, 0.15, 0.175% (w/v)), and pH of greater than or less than about 6 (e.g., within not less than 0.5 of 6.0) (e.g., about 6.5).

In an embodiment of the invention, the viscosity reducer (e.g., a dicarboxylic acid, an inorganic salt, an ester of citric acid, a xanthine, adipic acid; NaCl; caffeine; triethyl citrate, an amino acid, (D- or L-) arginine, L-arginine HCl, (D- or L-) alanine, (D- or L-) histidine, proline, (D- or L-) valine, glycine, (D- or L-) serine, (D- or L-) phenylalanine, (D- or L-) lysine, and (D- or L-) glutamate, and salts thereof; pyridoxamine; L-Ornithine; thiamine phosphoric acid ester chloride dihydrate, benzenesulfonic acid and/or pyridoxine; for example at a concentration of about 20-140, 20, 25, 30, 35, 40, 45, 50, 55, 60, 65, 70, 75, 80, 85, 90, 95, 100, 105, 110, 115, 120, 125, 130, 135 or 140 mM)) is at a concentration of about 5 mM to about 100 mM (e.g., 50-75 mM) each. If the viscosity reducer is an amino acid, it can be the L-enantiomer thereof or the D-enantiomer thereof. The viscosity reducer may be the conjugate base or salt thereof of an acid specified herein. In an embodiment of the invention, the co-formulation is characterized by about 96% or more anti-C5 antibody or antigen-binding fragment purity as assessed by size exclusion chromatography after about 1 month at 2-8° C.; and/or about 94% or more C5 iRNA purity as assessed by anion exchange chromatography after about 1 month at 2-8° C. In an embodiment of the invention, the co-formulation has a 1:1 ratio of milligrams per milliliter concentration of C5 iRNA and anti-C5 antibody or antigen-binding fragment; and, optionally, a viscosity reducer which is arginine, adipate, NaCl, lysine, aspartate, proline, histidine, caffeine, phenylalanine and/or triethyl citrate, e.g., at a concentration of about 75 mM arginine, 75 mM adipate, 75 mM NaCl, 75 mM lysine, 75 mM aspartate, 75 mM proline, 50 mM histidine (wherein, if the buffer is histidine based, then the total histidine concentration of the co-formulation is 50 mM), 50 mM caffeine, 50 mM phenylalanine and/or 75 mM triethyl citrate. In an embodiment of the invention, the co-formulation has a 1:2 ratio of milligrams per milliliter concentration of C5 iRNA and anti-C5 antibody or antigen-binding fragment; and, optionally, a viscosity reducer such as is arginine, adipate, NaCl, lysine and/or aspartate, e.g., at a concentration of about 75 mM arginine, 75 mM adipate, 75 mM NaCl, 75 mM lysine and/or 75 mM aspartate.

In an embodiment of the invention, the co-formulation includes an antibody or antigen-binding fragment thereof that binds specifically to C5 (anti-C5) including: a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10; a heavy chain variable region (HCVR) that comprises the HCDR1, HCDR2 and HCDR3 of a HCVR that comprises the amino acid sequence set forth in SEQ ID NO: 18, and a light chain variable region (LCVR) that comprises the LCDR1, LCDR2 and LCDR3 of a LCVR that comprises the amino acid sequence set forth in SEQ ID NO: 26; a heavy chain variable region (HCVR) that comprises the HCDR1, HCDR2 and HCDR3 of a HCVR that comprises the amino acid sequence set forth in SEQ ID NO: 34, and a light chain variable region (LCVR) that comprises the LCDR1, LCDR2 and LCDR3 of a LCVR that comprises the amino acid sequence set forth in SEQ ID NO: 42; a heavy chain variable region (HCVR) that comprises the HCDR1, HCDR2 and HCDR3 of a HCVR that comprises the amino acid sequence set forth in SEQ ID NO: 50, and a light chain variable region (LCVR) that comprises the LCDR1, LCDR2 and LCDR3 of a LCVR that comprises the amino acid sequence set forth in SEQ ID NO: 58; a heavy chain variable region (HCVR) that comprises the HCDR1, HCDR2 and HCDR3 of a HCVR that comprises the amino acid sequence set forth in SEQ ID NO: 66, and a light chain variable region (LCVR) that comprises the LCDR1, LCDR2 and LCDR3 of a LCVR that comprises the amino acid sequence set forth in SEQ ID NO: 74; a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90; a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 106; a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 114; a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 106; a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 130; a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 106; a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 106; a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130; a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 114; a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 130; a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 130; a heavy chain variable region (HCVR) that comprises the HCDR1, HCDR2 and HCDR3 of a HCVR that comprises the amino acid sequence set forth in SEQ ID NO: 154, and a light chain variable region (LCVR) that comprises the LCDR1, LCDR2 and LCDR3 of a LCVR that comprises the amino acid sequence set forth in SEQ ID NO: 162; a heavy chain variable region (HCVR) that comprises the HCDR1, HCDR2 and HCDR3 of a HCVR that comprises the amino acid sequence set forth in SEQ ID NO: 170, and a light chain variable region (LCVR) that comprises the LCDR1, LCDR2 and LCDR3 of a LCVR that comprises the amino acid sequence set forth in SEQ ID NO: 178; a heavy chain variable region (HCVR) that comprises the HCDR1, HCDR2 and HCDR3 of a HCVR that comprises the amino acid sequence set forth in SEQ ID NO: 186, and a light chain variable region (LCVR) that comprises the LCDR1, LCDR2 and LCDR3 of a LCVR that comprises the amino acid sequence set forth in SEQ ID NO: 194; a heavy chain variable region (HCVR) that comprises the HCDR1, HCDR2 and HCDR3 of a HCVR that comprises the amino acid sequence set forth in SEQ ID NO: 202, and a light chain variable region (LCVR) that comprises the LCDR1, LCDR2 and LCDR3 of a LCVR that comprises the amino acid sequence set forth in SEQ ID NO: 210; a heavy chain variable region (HCVR) that comprises the HCDR1, HCDR2 and HCDR3 of a HCVR that comprises the amino acid sequence set forth in SEQ ID NO: 218, and a light chain variable region (LCVR) that comprises the LCDR1, LCDR2 and LCDR3 of a LCVR that comprises the amino acid sequence set forth in SEQ ID NO: 226; a heavy chain variable region (HCVR) that comprises the HCDR1, HCDR2 and HCDR3 of a HCVR that comprises the amino acid sequence set forth in SEQ ID NO: 234, and a light chain variable region (LCVR) that comprises the LCDR1, LCDR2 and LCDR3 of a LCVR that comprises the amino acid sequence set forth in SEQ ID NO: 242; a heavy chain variable region (HCVR) that comprises the HCDR1, HCDR2 and HCDR3 of a HCVR that comprises the amino acid sequence set forth in SEQ ID NO: 250, and a light chain variable region (LCVR) that comprises the LCDR1, LCDR2 and LCDR3 of a LCVR that comprises the amino acid sequence set forth in SEQ ID NO: 258; a heavy chain variable region (HCVR) that comprises the HCDR1, HCDR2 and HCDR3 of a HCVR that comprises the amino acid sequence set forth in SEQ ID NO: 266, and a light chain variable region (LCVR) that comprises the LCDR1, LCDR2 and LCDR3 of a LCVR that comprises the amino acid sequence set forth in SEQ ID NO: 258; a heavy chain variable region (HCVR) that comprises the HCDR1, HCDR2 and HCDR3 of a HCVR that comprises the amino acid sequence set forth in SEQ ID NO: 274, and a light chain variable region (LCVR) that comprises the LCDR1, LCDR2 and LCDR3 of a LCVR that comprises the amino acid sequence set forth in SEQ ID NO: 282; a heavy chain variable region (HCVR) that comprises the HCDR1, HCDR2 and HCDR3 of a HCVR that comprises the amino acid sequence set forth in SEQ ID NO: 290, and a light chain variable region (LCVR) that comprises the LCDR1, LCDR2 and LCDR3 of a LCVR that comprises the amino acid sequence set forth in SEQ ID NO: 298; a heavy chain variable region (HCVR) that comprises the HCDR1, HCDR2 and HCDR3 of a HCVR that comprises the amino acid sequence set forth in SEQ ID NO: 306, and a light chain variable region (LCVR) that comprises the LCDR1, LCDR2 and LCDR3 of a LCVR that comprises the amino acid sequence set forth in SEQ ID NO: 314; a heavy chain variable region (HCVR) that comprises the HCDR1, HCDR2 and HCDR3 of a HCVR that comprises the amino acid sequence set forth in SEQ ID NO: 322, and a light chain variable region (LCVR) that comprises the LCDR1, LCDR2 and LCDR3 of a LCVR that comprises the amino acid sequence set forth in SEQ ID NO: 330; and/or a heavy chain variable region (HCVR) that comprises the HCDR1, HCDR2 and HCDR3 of a HCVR that comprises the amino acid sequence set forth in SEQ ID NO: 338, and a light chain variable region (LCVR) that comprises the LCDR1, LCDR2 and LCDR3 of a LCVR that comprises the amino acid sequence set forth in SEQ ID NO: 346. For example, in an embodiment of the invention, the antibody or antigen-binding fragment thereof that binds specifically to C5 (anti-C5) includes a heavy chain variable region comprising an HCDR1 that comprises the amino acid sequence set forth in SEQ ID NO: 4, an HCDR2 that comprises the amino acid sequence set forth in SEQ ID NO: 6, an HCDR3 that comprises the amino acid sequence set forth in SEQ ID NO: 8, and a light chain variable region comprising an LCDR1 that comprises the amino acid sequence set forth in SEQ ID NO: 12, an LCDR2 that comprises the amino acid sequence set forth in SEQ ID NO: 14, an LCDR3 that comprises the amino acid sequence set forth in SEQ ID NO: 16; a heavy chain variable region comprising an HCDR1 that comprises the amino acid sequence set forth in SEQ ID NO: 20, an HCDR2 that comprises the amino acid sequence set forth in SEQ ID NO: 22, an HCDR3 that comprises the amino acid sequence set forth in SEQ ID NO: 24, and a light chain variable region comprising an LCDR1 that comprises the amino acid sequence set forth in SEQ ID NO: 28, an LCDR2 that comprises the amino acid sequence set forth in SEQ ID NO: 30, an LCDR3 that comprises the amino acid sequence set forth in SEQ ID NO: 32; a heavy chain variable region comprising an HCDR1 that comprises the amino acid sequence set forth in SEQ ID NO: 36, an HCDR2 that comprises the amino acid sequence set forth in SEQ ID NO: 38, an HCDR3 that comprises the amino acid sequence set forth in SEQ ID NO: 40, and a light chain variable region comprising an LCDR1 that comprises the amino acid sequence set forth in SEQ ID NO: 44, an LCDR2 that comprises the amino acid sequence set forth in SEQ ID NO: 46, an LCDR3 that comprises the amino acid sequence set forth in SEQ ID NO: 48; a heavy chain variable region comprising an HCDR1 that comprises the amino acid sequence set forth in SEQ ID NO: 52, an HCDR2 that comprises the amino acid sequence set forth in SEQ ID NO: 54, an HCDR3 that comprises the amino acid sequence set forth in SEQ ID NO: 56, and a light chain variable region comprising an LCDR1 that comprises the amino acid sequence set forth in SEQ ID NO: 60, an LCDR2 that comprises the amino acid sequence set forth in SEQ ID NO: 62, an LCDR3 that comprises the amino acid sequence set forth in SEQ ID NO: 64; a heavy chain variable region comprising an HCDR1 that comprises the amino acid sequence set forth in SEQ ID NO: 68, an HCDR2 that comprises the amino acid sequence set forth in SEQ ID NO: 70, an HCDR3 that comprises the amino acid sequence set forth in SEQ ID NO: 72, and a light chain variable region comprising an LCDR1 that comprises the amino acid sequence set forth in SEQ ID NO: 76, an LCDR2 that comprises the amino acid sequence set forth in SEQ ID NO: 78, an LCDR3 that comprises the amino acid sequence set forth in SEQ ID NO: 80; a heavy chain variable region comprising an HCDR1 that comprises the amino acid sequence set forth in SEQ ID NO: 84, an HCDR2 that comprises the amino acid sequence set forth in SEQ ID NO: 86, an HCDR3 that comprises the amino acid sequence set forth in SEQ ID NO: 88, and a light chain variable region comprising an LCDR1 that comprises the amino acid sequence set forth in SEQ ID NO: 92, an LCDR2 that comprises the amino acid sequence set forth in SEQ ID NO: 94, an LCDR3 that comprises the amino acid sequence set forth in SEQ ID NO: 96; a heavy chain variable region comprising an HCDR1 that comprises the amino acid sequence set forth in SEQ ID NO: 100, an HCDR2 that comprises the amino acid sequence set forth in SEQ ID NO: 102, an HCDR3 that comprises the amino acid sequence set forth in SEQ ID NO: 104, and a light chain variable region comprising an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a heavy chain variable region comprising an HCDR1 that comprises the amino acid sequence set forth in SEQ ID NO: 100, an HCDR2 that comprises the amino acid sequence set forth in SEQ ID NO: 102, an HCDR3 that comprises the amino acid sequence set forth in SEQ ID NO: 104, and a light chain variable region comprising an LCDR1 that comprises the amino acid sequence set forth in SEQ ID NO: 116, an LCDR2 that comprises the amino acid sequence set forth in SEQ ID NO: 118, an LCDR3 that comprises the amino acid sequence set forth in SEQ ID NO: 120; a heavy chain variable region comprising an HCDR1 that comprises the amino acid sequence set forth in SEQ ID NO: 124, an HCDR2 that comprises the amino acid sequence set forth in SEQ ID NO: 126, an HCDR3 that comprises the amino acid sequence set forth in SEQ ID NO: 128, and a light chain variable region comprising an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a heavy chain variable region comprising an HCDR1 that comprises the amino acid sequence set forth in SEQ ID NO: 100, an HCDR2 that comprises the amino acid sequence set forth in SEQ ID NO: 102, an HCDR3 that comprises the amino acid sequence set forth in SEQ ID NO: 104, and a light chain variable region comprising an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a heavy chain variable region comprising an HCDR1 that comprises the amino acid sequence set forth in SEQ ID NO: 140, an HCDR2 that comprises the amino acid sequence set forth in SEQ ID NO: 142, an HCDR3 that comprises the amino acid sequence set forth in SEQ ID NO: 144, and a light chain variable region comprising an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a heavy chain variable region comprising an HCDR1 that comprises the amino acid sequence set forth in SEQ ID NO: 148, an HCDR2 that comprises the amino acid sequence set forth in SEQ ID NO: 150, an HCDR3 that comprises the amino acid sequence set forth in SEQ ID NO: 152, and a light chain variable region comprising an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a heavy chain variable region comprising an HCDR1 that comprises the amino acid sequence set forth in SEQ ID NO: 124, an HCDR2 that comprises the amino acid sequence set forth in SEQ ID NO: 126, an HCDR3 that comprises the amino acid sequence set forth in SEQ ID NO: 128, and a light chain variable region comprising an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a heavy chain variable region comprising an HCDR1 that comprises the amino acid sequence set forth in SEQ ID NO: 148, an HCDR2 that comprises the amino acid sequence set forth in SEQ ID NO: 150, an HCDR3 that comprises the amino acid sequence set forth in SEQ ID NO: 152, and a light chain variable region comprising an LCDR1 that comprises the amino acid sequence set forth in SEQ ID NO: 116, an LCDR2 that comprises the amino acid sequence set forth in SEQ ID NO: 118, an LCDR3 that comprises the amino acid sequence set forth in SEQ ID NO: 120; a heavy chain variable region comprising an HCDR1 that comprises the amino acid sequence set forth in SEQ ID NO: 148, an HCDR2 that comprises the amino acid sequence set forth in SEQ ID NO: 150, an HCDR3 that comprises the amino acid sequence set forth in SEQ ID NO: 152, and a light chain variable region comprising an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a heavy chain variable region comprising an HCDR1 that comprises the amino acid sequence set forth in SEQ ID NO: 140, an HCDR2 that comprises the amino acid sequence set forth in SEQ ID NO: 142, an HCDR3 that comprises the amino acid sequence set forth in SEQ ID NO: 144, and a light chain variable region comprising an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a heavy chain variable region comprising an HCDR1 that comprises the amino acid sequence set forth in SEQ ID NO: 156, an HCDR2 that comprises the amino acid sequence set forth in SEQ ID NO: 158, an HCDR3 that comprises the amino acid sequence set forth in SEQ ID NO: 160, and a light chain variable region comprising an LCDR1 that comprises the amino acid sequence set forth in SEQ ID NO: 164, an LCDR2 that comprises the amino acid sequence set forth in SEQ ID NO: 166, an LCDR3 that comprises the amino acid sequence set forth in SEQ ID NO: 168; a heavy chain variable region comprising an HCDR1 that comprises the amino acid sequence set forth in SEQ ID NO: 172, an HCDR2 that comprises the amino acid sequence set forth in SEQ ID NO: 174, an HCDR3 that comprises the amino acid sequence set forth in SEQ ID NO: 176, and a light chain variable region comprising an LCDR1 that comprises the amino acid sequence set forth in SEQ ID NO: 180, an LCDR2 that comprises the amino acid sequence set forth in SEQ ID NO: 182, an LCDR3 that comprises the amino acid sequence set forth in SEQ ID NO: 184; a heavy chain variable region comprising an HCDR1 that comprises the amino acid sequence set forth in SEQ ID NO: 188, an HCDR2 that comprises the amino acid sequence set forth in SEQ ID NO: 190, an HCDR3 that comprises the amino acid sequence set forth in SEQ ID NO: 192, and a light chain variable region comprising an LCDR1 that comprises the amino acid sequence set forth in SEQ ID NO: 196, an LCDR2 that comprises the amino acid sequence set forth in SEQ ID NO: 198, an LCDR3 that comprises the amino acid sequence set forth in SEQ ID NO: 200; a heavy chain variable region comprising an HCDR1 that comprises the amino acid sequence set forth in SEQ ID NO: 204, an HCDR2 that comprises the amino acid sequence set forth in SEQ ID NO: 206, an HCDR3 that comprises the amino acid sequence set forth in SEQ ID NO: 208, and a light chain variable region comprising an LCDR1 that comprises the amino acid sequence set forth in SEQ ID NO: 212, an LCDR2 that comprises the amino acid sequence set forth in SEQ ID NO: 214, an LCDR3 that comprises the amino acid sequence set forth in SEQ ID NO: 216; a heavy chain variable region comprising an HCDR1 that comprises the amino acid sequence set forth in SEQ ID NO: 220, an HCDR2 that comprises the amino acid sequence set forth in SEQ ID NO: 222, an HCDR3 that comprises the amino acid sequence set forth in SEQ ID NO: 224, and a light chain variable region comprising an LCDR1 that comprises the amino acid sequence set forth in SEQ ID NO: 228, an LCDR2 that comprises the amino acid sequence set forth in SEQ ID NO: 230, an LCDR3 that comprises the amino acid sequence set forth in SEQ ID NO: 232; a heavy chain variable region comprising an HCDR1 that comprises the amino acid sequence set forth in SEQ ID NO: 236, an HCDR2 that comprises the amino acid sequence set forth in SEQ ID NO: 238, an HCDR3 that comprises the amino acid sequence set forth in SEQ ID NO: 240, and a light chain variable region comprising an LCDR1 that comprises the amino acid sequence set forth in SEQ ID NO: 244, an LCDR2 that comprises the amino acid sequence set forth in SEQ ID NO: 246, an LCDR3 that comprises the amino acid sequence set forth in SEQ ID NO: 248; a heavy chain variable region comprising an HCDR1 that comprises the amino acid sequence set forth in SEQ ID NO: 252, an HCDR2 that comprises the amino acid sequence set forth in SEQ ID NO: 254, an HCDR3 that comprises the amino acid sequence set forth in SEQ ID NO: 256, and a light chain variable region comprising an LCDR1 that comprises the amino acid sequence set forth in SEQ ID NO: 260, an LCDR2 that comprises the amino acid sequence set forth in SEQ ID NO: 262, an LCDR3 that comprises the amino acid sequence set forth in SEQ ID NO: 264; a heavy chain variable region comprising an HCDR1 that comprises the amino acid sequence set forth in SEQ ID NO: 268, an HCDR2 that comprises the amino acid sequence set forth in SEQ ID NO: 270, an HCDR3 that comprises the amino acid sequence set forth in SEQ ID NO: 272, and a light chain variable region comprising an LCDR1 that comprises the amino acid sequence set forth in SEQ ID NO: 260, an LCDR2 that comprises the amino acid sequence set forth in SEQ ID NO: 262, an LCDR3 that comprises the amino acid sequence set forth in SEQ ID NO: 264; a heavy chain variable region comprising an HCDR1 that comprises the amino acid sequence set forth in SEQ ID NO: 276, an HCDR2 that comprises the amino acid sequence set forth in SEQ ID NO: 278, an HCDR3 that comprises the amino acid sequence set forth in SEQ ID NO: 280, and a light chain variable region comprising an LCDR1 that comprises the amino acid sequence set forth in SEQ ID NO: 284, an LCDR2 that comprises the amino acid sequence set forth in SEQ ID NO: 286, an LCDR3 that comprises the amino acid sequence set forth in SEQ ID NO: 288; a heavy chain variable region comprising an HCDR1 that comprises the amino acid sequence set forth in SEQ ID NO: 292, an HCDR2 that comprises the amino acid sequence set forth in SEQ ID NO: 294, an HCDR3 that comprises the amino acid sequence set forth in SEQ ID NO: 296, and a light chain variable region comprising an LCDR1 that comprises the amino acid sequence set forth in SEQ ID NO: 300, an LCDR2 that comprises the amino acid sequence set forth in SEQ ID NO: 302, an LCDR3 that comprises the amino acid sequence set forth in SEQ ID NO: 304; a heavy chain variable region comprising an HCDR1 that comprises the amino acid sequence set forth in SEQ ID NO: 308, an HCDR2 that comprises the amino acid sequence set forth in SEQ ID NO: 310, an HCDR3 that comprises the amino acid sequence set forth in SEQ ID NO: 312, and a light chain variable region comprising an LCDR1 that comprises the amino acid sequence set forth in SEQ ID NO: 316, an LCDR2 that comprises the amino acid sequence set forth in SEQ ID NO: 318, an LCDR3 that comprises the amino acid sequence set forth in SEQ ID NO: 320; a heavy chain variable region comprising an HCDR1 that comprises the amino acid sequence set forth in SEQ ID NO: 324, an HCDR2 that comprises the amino acid sequence set forth in SEQ ID NO: 326, an HCDR3 that comprises the amino acid sequence set forth in SEQ ID NO: 328, and a light chain variable region comprising an LCDR1 that comprises the amino acid sequence set forth in SEQ ID NO: 332, an LCDR2 that comprises the amino acid sequence set forth in SEQ ID NO: 334, an LCDR3 that comprises the amino acid sequence set forth in SEQ ID NO: 336; or a heavy chain variable region comprising an HCDR1 that comprises the amino acid sequence set forth in SEQ ID NO: 340, an HCDR2 that comprises the amino acid sequence set forth in SEQ ID NO: 342, an HCDR3 that comprises the amino acid sequence set forth in SEQ ID NO: 344, and a light chain variable region comprising an LCDR1 that comprises the amino acid sequence set forth in SEQ ID NO: 348, an LCDR2 that comprises the amino acid sequence set forth in SEQ ID NO: 350, an LCDR3 that comprises the amino acid sequence set forth in SEQ ID NO: 352. In an embodiment of the invention, the antibody or antigen-binding fragment thereof that binds specifically to C5 (anti-C5) includes a heavy chain variable region that comprises the amino acid sequence set forth in SEQ ID NO: 2, a light chain variable region that comprises the amino acid sequence set forth in SEQ ID NO: 10; a heavy chain variable region that comprises the amino acid sequence set forth in SEQ ID NO: 18, a light chain variable region that comprises the amino acid sequence set forth in SEQ ID NO: 26; a heavy chain variable region that comprises the amino acid sequence set forth in SEQ ID NO: 34, a light chain variable region that comprises the amino acid sequence set forth in SEQ ID NO: 42; a heavy chain variable region that comprises the amino acid sequence set forth in SEQ ID NO: 50, a light chain variable region that comprises the amino acid sequence set forth in SEQ ID NO: 58; a heavy chain variable region that comprises the amino acid sequence set forth in SEQ ID NO: 66, a light chain variable region that comprises the amino acid sequence set forth in SEQ ID NO: 74; a heavy chain variable region that comprises the amino acid sequence set forth in SEQ ID NO: 82, a light chain variable region that comprises the amino acid sequence set forth in SEQ ID NO: 90; a heavy chain variable region that comprises the amino acid sequence set forth in SEQ ID NO: 98, a light chain variable region that comprises the amino acid sequence set forth in SEQ ID NO: 106; a heavy chain variable region that comprises the amino acid sequence set forth in SEQ ID NO: 98, a light chain variable region that comprises the amino acid sequence set forth in SEQ ID NO: 114; a heavy chain variable region that comprises the amino acid sequence set forth in SEQ ID NO: 122, a light chain variable region that comprises the amino acid sequence set forth in SEQ ID NO: 106; a heavy chain variable region that comprises the amino acid sequence set forth in SEQ ID NO: 98, a light chain variable region that comprises the amino acid sequence set forth in SEQ ID NO: 130; a heavy chain variable region that comprises the amino acid sequence set forth in SEQ ID NO: 138, a light chain variable region that comprises the amino acid sequence set forth in SEQ ID NO: 106; a heavy chain variable region that comprises the amino acid sequence set forth in SEQ ID NO: 146, a light chain variable region that comprises the amino acid sequence set forth in SEQ ID NO: 106; a heavy chain variable region that comprises the amino acid sequence set forth in SEQ ID NO: 122, a light chain variable region that comprises the amino acid sequence set forth in SEQ ID NO: 130; a heavy chain variable region that comprises the amino acid sequence set forth in SEQ ID NO: 146, a light chain variable region that comprises the amino acid sequence set forth in SEQ ID NO: 114; a heavy chain variable region that comprises the amino acid sequence set forth in SEQ ID NO: 146, a light chain variable region that comprises the amino acid sequence set forth in SEQ ID NO: 130; a heavy chain variable region that comprises the amino acid sequence set forth in SEQ ID NO: 138, a light chain variable region that comprises the amino acid sequence set forth in SEQ ID NO: 130; a heavy chain variable region that comprises the amino acid sequence set forth in SEQ ID NO: 154, a light chain variable region that comprises the amino acid sequence set forth in SEQ ID NO: 162; a heavy chain variable region that comprises the amino acid sequence set forth in SEQ ID NO: 170, a light chain variable region that comprises the amino acid sequence set forth in SEQ ID NO: 178; a heavy chain variable region that comprises the amino acid sequence set forth in SEQ ID NO: 186, a light chain variable region that comprises the amino acid sequence set forth in SEQ ID NO: 194; a heavy chain variable region that comprises the amino acid sequence set forth in SEQ ID NO: 202, a light chain variable region that comprises the amino acid sequence set forth in SEQ ID NO: 210; a heavy chain variable region that comprises the amino acid sequence set forth in SEQ ID NO: 218, a light chain variable region that comprises the amino acid sequence set forth in SEQ ID NO: 226; a heavy chain variable region that comprises the amino acid sequence set forth in SEQ ID NO: 234, a light chain variable region that comprises the amino acid sequence set forth in SEQ ID NO: 242; a heavy chain variable region that comprises the amino acid sequence set forth in SEQ ID NO: 250, a light chain variable region that comprises the amino acid sequence set forth in SEQ ID NO: 258; a heavy chain variable region that comprises the amino acid sequence set forth in SEQ ID NO: 266, a light chain variable region that comprises the amino acid sequence set forth in SEQ ID NO: 258; a heavy chain variable region that comprises the amino acid sequence set forth in SEQ ID NO: 274, a light chain variable region that comprises the amino acid sequence set forth in SEQ ID NO: 282; a heavy chain variable region that comprises the amino acid sequence set forth in SEQ ID NO: 290, a light chain variable region that comprises the amino acid sequence set forth in SEQ ID NO: 298; a heavy chain variable region that comprises the amino acid sequence set forth in SEQ ID NO: 306, a light chain variable region that comprises the amino acid sequence set forth in SEQ ID NO: 314; a heavy chain variable region that comprises the amino acid sequence set forth in SEQ ID NO: 322, a light chain variable region that comprises the amino acid sequence set forth in SEQ ID NO: 330; or a heavy chain variable region that comprises the amino acid sequence set forth in SEQ ID NO: 338, a light chain variable region that comprises the amino acid sequence set forth in SEQ ID NO: 346. For example, in an embodiment of the invention, the co-formulation includes about 90 to about 275 mg/ml; or about 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 211, 220, 242, or 274 mg/ml; or at least about 150 mg/ml, at least about 175 mg/ml, at least about 200 mg/ml, at least about 211 mg/ml, at least about 220 mg/ml, at least about 242 mg/ml or at least about 274 mg/ml of the antibody or antigen-binding fragment that specifically binds to C5 (anti-C5).

In an embodiment of the invention, the co-formulation includes a C5 iRNA that is a double-stranded ribonucleic acid (dsRNA) agent comprising a sense strand and an antisense strand, wherein the antisense strand comprises a region of complementarity comprising at least 17 contiguous nucleotides differing by no more than 3 nucleotides from the nucleotide sequence of 5′-UAUUAUAAAAAUAUCUUGCUUUU-3′ (SEQ ID NO: 364), and wherein the dsRNA agent comprises at least one modified nucleotide. In an embodiment of the invention, the co-formulation includes a C5 iRNA that is a double-stranded ribonucleic acid (dsRNA) agent comprising a sense strand and an antisense strand, wherein the sense strand comprises 5′-asasGfcAfaGfaUfAfUfuUfuuAfuAfaua-3′ (SEQ ID NO: 406) and the antisense strand comprises 5′-usAfsUfuAfuaAfaAfauaUfcUfuGfcuususudTdT-3′ (SEQ ID NO: 369), wherein a, g, c and u are 2′-O-methyl (2′-OMe) A, G, C, and U, respectively; Af, Gf, Cf and Uf are 2′-fluoro A, G, C and U, respectively; dT is a deoxy-thymine nucleotide; s is a phosphorothioate linkage; and wherein the sense strand is conjugated at the 3′-terminus to the ligand

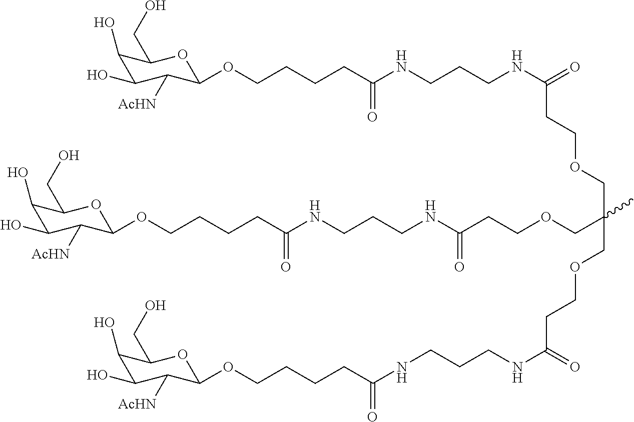 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

(e.g., wherein the C5 iRNA is Cemdisiran). In an embodiment of the invention, the co-formulation includes a C5 iRNA which is Cemdisiran and one or more of Cemdisiran impurity 1, Cemdisiran impurity 2 and Cemdisiran impurity 3 as discussed herein. In an embodiment of the invention, the C5 iRNA is at a concentration of about 20-100, 20, 25, 30, 35, 40, 45, 50, 55, 60, 65, 70, 75, 80, 85, 90, 95, 100, 110, 115, 120, 130, 140, 150, 155, 160, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg/ml.

In an embodiment of the invention, the co-formulation is characterized by a viscosity <30 cP at 20° C.; and/or an osmolality of 240-450 mOsm/kg; e.g. a viscosity ≤20 cP at 20° C.

The present invention includes a co-formulation including any of the following:
a double stranded C5 iRNA; and
an anti-C5 antibody or antigen-binding fragment thereof,
a pH above or below (by at least 0.5) 6.0 (e.g., about 6.5);
a C5 iRNA,
an anti-C5 antibody or antigen-binding fragment thereof,
a buffer,
a viscosity reducer,
a stabilizer, and
a non-ionic surfactant;
a C5 iRNA,
an anti-C5 antibody or antigen-binding fragment thereof,
histidine-based buffer,
L-arginine,
a stabilizer, and
a non-ionic surfactant;
a C5 iRNA,
an anti-C5 antibody or antigen-binding fragment thereof,
histidine-based buffer,
L-arginine,
a sugar or polyol, and
a non-ionic surfactant;
Cemdisiran,
Pozelimab,
histidine-based buffer,
L-arginine,
a stabilizer, and
a non-ionic surfactant,
pH about 6.5;
Cemdisiran,
Pozelimab
histidine-based buffer,
L-arginine,
sucrose, and
polysorbate 80,
pH about 6.5;
100 (±10) mg/mL C5 iRNA,
100 (±10) mg/mL anti-C5 antibody or antigen-binding fragment thereof,
50 (±5) mM viscosity reducer,
10 (±1) mM buffer,
1.0 (±0.1)% stabilizer,
0.075 (±0.0075)% non-ionic surfactant,
pH about 6.5;
75 (±7.5) mg/mL C5 iRNA,
150 (±15) mg/mL anti-C5 antibody or antigen-binding fragment thereof,
75 (±7.5) mM viscosity reducer,
15 (±1.5) mM buffer,
1.5 (±0.15)% stabilizer,
0.1125 (±0.01125)% non-ionic surfactant,
pH about 6.5;
50 (±5) mg/mL C5 iRNA,
100 (±10) mg/mL anti-C5 antibody or antigen-binding fragment thereof,
75 mM (±7.5) viscosity reducer,
15 (±1.5) mM buffer,
1.5 (±0.15)% stabilizer,
0.1125 (±0.01125)% non-ionic surfactant;
pH about 6.5;
50 (±5) mg/mL C5 iRNA,
100 (±10) mg/mL anti-C5 antibody or antigen-binding fragment thereof,
75 (±7.5) mM viscosity reducer,
35 (±3.5) mM buffer,
1.5 (±0.15)% stabilizer,
0.1125 (±0.01125)% non-ionic surfactant,
pH about 6.5;
100 (±10) mg/mL C5 iRNA,
100 (±10) mg/mL anti-C5 antibody or antigen-binding fragment thereof,
50 (±5) mM viscosity reducer,
30 (±3) mM buffer,
1 (±0.1)% stabilizer,
0.075 (±0.0075)% non-ionic surfactant,
pH about 6.5;
50 (±5) mg/mL C5 iRNA,
100 (±10) mg/mL anti-C5 antibody or antigen-binding fragment thereof,
90 (±9) mM viscosity reducer,
30 (±3) mM buffer,
1 (±0.1)% stabilizer,
0.075 (±0.0075)% non-ionic surfactant,
pH about 6.5;
about 100 mg/mL Cemdisiran,
about 100 mg/mL Pozelimab,
about 50 mM L-arginine,
about 30 mM histidine-based buffer,
about 1% (w/v) sucrose,
about 0.075% (w/v) PS80,
pH about 6.5;
about 50 mg/mL Cemdisiran,
about 100 mg/mL Pozelimab,
about 90 mM L-arginine,
about 30 mM histidine-based buffer,
about 1% (w/v) sucrose,
about 0.075% (w/v) PS80,
pH about 6.5;
about 100 mg/mL Cemdisiran,
about 100 mg/mL Pozelimab,
about 50 mM L-arginine,
about 10 mM histidine-based buffer,
about 1.0% sucrose,
about 0.075% PS80,
pH about 6.5;
about 75 mg/mL Cemdisiran,
about 150 mg/mL Pozelimab,
about 75 mM L-arginine,
about 15 mM histidine-based buffer,
about 1.5% sucrose,
about 0.1125% PS80,
pH about 6.5;
about 50 mg/mL Cemdisiran,
about 100 mg/mL Pozelimab,
about 75 mM L-arginine,
about 15 mM histidine-based buffer,
about 1.5% sucrose,
about 0.1125% PS80;
pH about 6.5;
about 50 mg/mL Cemdisiran,
about 100 mg/mL Pozelimab,
about 75 mM L-arginine,
about 35 mM histidine-based buffer,
about 1.5% sucrose,
about 0.1125% PS80,
pH about 6.5;
about 100 mg/mL Cemdisiran,
about 100 mg/mL Pozelimab,
about 50 mM L-arginine,
about 30 mM histidine-based buffer,
about 1% sucrose,
about 0.075% PS80,
pH about 6.5;
about 50 mg/mL Cemdisiran,
about 100 mg/mL Pozelimab,
about 90 mM L-arginine,
about 30 mM histidine-based buffer,
about 1% sucrose,
about 0.075% PS80,
pH about 6.5;
optionally, further comprising GalNAc and/or GlcNAc;
about 120 mg/mL C5 iRNA,
about 120 mg/mL anti-C5 antibody or antigen-binding fragment,
A viscosity reducer;
about 15 mM histidine,
pH about 6.2;
about 75 mg/mL C5 iRNA,
about 150 mg/mL anti-C5 antibody or antigen-binding fragment,
A viscosity reducer;
about 15 mM histidine,
pH about 6.2;
about 120 mg/mL C5 iRNA,
about 120 mg/mL anti-C5 antibody or antigen-binding fragment,
about 15 mM histidine,
pH about 6.2;
about 75 mg/mL C5 iRNA,
about 150 mg/mL anti-C5 antibody or antigen-binding fragment,
about 15 mM histidine,
pH about 6.2;
about 120 mg/mL Cemdisiran,
about 120 mg/mL Pozelimab,
about 15 mM histidine,
pH about 6.2;
about 120 mg/mL Cemdisiran,
about 120 mg/mL Pozelimab,
about 75 mM arginine,
about 15 mM histidine,
pH about 6.2;
about 120 mg/mL Cemdisiran,
about 120 mg/mL Pozelimab,
about 75 mM adipate,
about 15 mM histidine,
pH about 6.2;
about 120 mg/mL Cemdisiran,
about 120 mg/mL Pozelimab,
about 75 mM NaCl,
about 15 mM histidine,
pH about 6.2;
about 120 mg/mL Cemdisiran,
about 120 mg/mL Pozelimab,
about 75 mM lysine,
about 15 mM histidine,
pH about 6.2;
about 120 mg/mL Cemdisiran,
about 120 mg/mL Pozelimab,
about 75 mM aspartate,
about 15 mM histidine,
pH about 6.2;
about 120 mg/mL Cemdisiran,
about 120 mg/mL Pozelimab,
about 75 mM proline,
about 15 mM histidine,
pH about 6.2;
about 120 mg/mL Cemdisiran,
about 120 mg/mL Pozelimab,
about 50 mM histidine,
pH about 6.2;
about 120 mg/mL Cemdisiran,
about 120 mg/mL Pozelimab,
about 50 mM caffeine,
about 15 mM histidine,
pH about 6.2;
about 120 mg/mL Cemdisiran,
about 120 mg/mL Pozelimab,
about 50 mM phenylalanine,
about 15 mM histidine,
pH about 6.2
about 120 mg/mL Cemdisiran,
about 120 mg/mL Pozelimab,
about 50 mM triethyl citrate,
about 15 mM histidine,
pH about 6.2;
about 75 mg/mL Cemdisiran,
about 150 mg/mL Pozelimab,
about 15 mM histidine,
pH about 6.2;
about 75 mg/mL Cemdisiran,
about 150 mg/mL Pozelimab,
about 75 mM arginine,
about 15 mM histidine,
pH about 6.2;
about 75 mg/mL Cemdisiran,
about 150 mg/mL Pozelimab,
about 75 mM adipate,
about 15 mM histidine,
pH about 6.2;
about 75 mg/mL Cemdisiran,
about 150 mg/mL Pozelimab,
about 75 mM NaCl,
about 15 mM histidine,
pH about 6.2;
about 75 mg/mL Cemdisiran,
about 150 mg/mL Pozelimab,
about 75 mM lysine,
about 15 mM histidine,
pH about 6.2;
or
about 75 mg/mL Cemdisiran,
about 150 mg/mL Pozelimab,
about 75 mM aspartate,
about 15 mM histidine,
pH about 6.2.

The present invention includes a co-formulation including a C5 iRNA which is Cemdisiran; an antibody or antigen-binding fragment which is Pozelimab; a viscosity reducer, which is L-arginine; a buffer which is a histidine-based buffer; a stabilizer which is sucrose; a non-ionic surfactant which is polysorbate 80; and a pH of about 6.5.

In an embodiment of the invention, the co-formulation includes a C5 iRNA that is conjugated to a ligand that comprises one or more terminal N-Acetylgalactosamine (GalNAc) or N-acetylglucosamine (GlcNAc) residues; has a pH that is within no less than about 0.5 of about 6; and/or a pH that is about 6.5.

In an embodiment of the invention, the co-formulation is characterized by one or more of: comprises beta-hexosaminidase; comprises the antibody of antigen-binding fragment thereof which was expressed and isolated from a mammalian host cell that contains beta-hexosaminidase; comprises the antibody of antigen-binding fragment thereof which was expressed and isolated from a Chinese hamster ovary cell; comprises no more than about 1% Cemdisiran Impurity 1 relative to total Cemdisiran; comprises no less than about 80% Cemdisiran, relative to total Cemdisiran, after 2 years storage at 2-8° C.; has about 91% Cemdisiran before storage (at t=0); has no less than about 80% Cemdisiran after 1, 1%, 2, 2% or 3 years storage at 2-8° C.; has about 80% to about 91% Cemdisiran; exhibits a Cemdisiran Purity (%) by dIPRP of about 90.5% at t=0; 91.1% after 1 month storage at 2-8° C.; 90.8% after 3 months storage at 2-8° C.; 90% after 6 months storage at 2-8° C.; 88.8% after 9 months storage at 2-8° C.; 88.7% after 12 months storage at 2-8° C.; 89% after 18 months storage at 2-8° C.; and/or 89.4% after 24 months storage at 2-8° C.; exhibits a Cemdisiran Purity (%) by dIPRP of about 90.8% at t=0, 90.6% after 1 month storage at 2-8° C.; 90.5% after 3 months storage at 2-8° C.; 89.4% after 6 months storage at 2-8° C.; 88.3% after 9 months storage at 2-8° C.; 87.8% after 12 months storage at 2-8° C.; 87.8% after 18 months storage at 2-8° C.; and/or 89.4% after 24 months storage at 2-8° C.; exhibits a Cemdisiran Single Strand Purity (%) by dIPRP of about 90.5% at t=0; 90.2% after 1 month storage at 25° C. and 60% RH; 87.8% after 3 months storage at 25° C. and 60% RH; 85.1% after 6 months storage at 25° C. and 60% RH; 90% after 0.5 months storage at 40° C. and 75% RH; 88.9% after 1 month storage at 40° C. and 75% RH; 85.8% after 3 months storage at 40° C. and 75% RH; exhibits a Cemdisiran Purity (%) by dIPRP of about 90.8% at t=0; 88.8% after 1 month storage at 25° C. and 60% RH; 85.9% after 3 months storage at 25° C. and 60% RH; 82.3% after 6 months storage at 25° C. and 60% RH; 88.9% after 0.5 months storage at 40° C. and 75% RH; 87.3% after 1 month storage at 40° C. and 75% RH; 82.3% after 3 months storage at 40° C. and 75% RH; exhibits a Cemdisiran purity (%) by dIPRP of about 90.9% at t=0; about 90.1% after 1 month of storage at 25° C., 60% RH; about 90.9% after 3 months of storage at 25° C., 60% RH; about 90.4% after 6 months of storage at 25° C., 60% RH; about 89.9% after 0.5 months of storage at 40° C., 75% RH; about 89.7% after 1 month of storage at 40° C., 75% RH; and/or about 89.5% after 3 months of storage at 40° C., 75% RH; exhibits a Cemdisiran purity (%) by dIPRP of about 90.8% at t=0; about 90.2% after 1 month of storage at 25° C., 60% RH; about 90.8% after 3 months of storage at 25° C., 60% RH; about 90.3% after 6 months of storage at 25° C., 60% RH; about 89.5% after 0.5 months of storage at 40° C., 75% RH; about 89.6% after 1 month of storage at 40° C., 75% RH; and/or about 89.1% after 3 months of storage at 40° C., 75% RH; exhibits a Cemdisiran purity (%) by dIPRP of about 90.5% at t=0; about 89.9% after 1 month of storage at 25° C., 60% RH; about 90.8% after 3 months of storage at 25° C., 60% RH; about 90.4% after 6 months of storage at 25° C., 60% RH; about 90.1% after 0.5 months of storage at 40° C., 75% RH; about 89.6% after 1 month of storage at 40° C., 75% RH; and/or about 89.9% after 3 months of storage at 40° C., 75% RH; and/or exhibits a Cemdisiran purity (%) by dIPRP of about 91.1% at t=0; about 90% after 1 month of storage at 25° C., 60% RH; about 91% after 3 months of storage at 25° C., 60% RH; about 90.7% after 6 months of storage at 25° C., 60% RH; about 90% after 0.5 months of storage at 40° C., 75% RH; about 89.7% after 1 month of storage at 40° C., 75% RH; and/or about 89.9% after 3 months of storage at 40° C., 75% RH. In an embodiment of the invention, the co-formulation is characterized by one or more of no more than about 2.1 parts per million (ppm) molar ratio of beta-hexosaminidase to antibody or antigen-binding fragment; include no more than about 0.170 micrograms/ml beta-hexosaminidase, include no more than about 0.04 micrograms/ml beta-hexosaminidase; and/or about 0.04; 0.05; 0.06; 0.06; 0.0605; 0.0605; 0.0605; 0.063; 0.07; 0.07; 0.0765; 0.078; 0.08; 0.14; 0.141; 0.15; 0.1525; 0.166; or 0.17 micrograms/ml beta-hexosaminidase; or no more than any of such concentrations.

The present invention also includes a method for administering a co-formulation as set forth herein to a subject comprising introducing the co-formulation into the body of the subject, for example, by injecting the co-formulation into the body of the subject; e.g., by intramuscular, subcutaneous, intravenous, intraocular and/or intravitreal injection.

The present invention also includes a method for treating or preventing a C5-associated disease or disorder (e.g., a disorder of inappropriate or undesirable complement activation; a hemodialysis complication; a lung disease or disorder; a neurological disorder; a parasitic disease; a post-ischemic reperfusion condition; a proteinuric kidney disease; a renal disorder; adult respiratory distress syndrome (ARDS); age-related macular degeneration (AMD); allergy; Alport's syndrome; Alzheimer's disease; an autoimmune disease; an immune complex disorder; an inflammatory disorder; an ocular disease; an organic dust disease; angiopathic thrombosis and protein-losing enteropathy; asthma; atherosclerosis; bronchoconstriction; bullous pemphigoid; C3 glomerulopathy; capillary leak syndrome; CHAPLE disease (CD55 deficiency with hyperactivation of complement; chemical injury due to irritant gasses and/or chemicals; chronic obstructive pulmonary disease (COPD); complement activation due to burn; complement activation due to frostbite; complement activation due to obesity; complement activation due to sepsis; Crohn's disease; diabetes; diabetic macular edema (DME); diabetic nephropathy; diabetic retinopathy; dry AMD; dyspnea; emphysema; epilepsy; fibrogenic dust diseases; geographic atrophy (GA); glomerulopathy; Goodpasture's Syndrome; Guillain-Barre Syndrome; hemolytic anemia; hemoptysis; hereditary angioedema; hyperacute allograft rejection; hypersensitivity pneumonitis; immune complex-associated inflammation; infectious disease; inflammation of an autoimmune disease; inherited CD59 deficiency; injury due to inert dusts and/or minerals; interleukin-2 induced toxicity during IL-2 therapy; lupus nephritis; membranoproliferative glomerulonephritis; membranoproliferative nephritis; mesenteric artery reperfusion after aortic reconstruction; multiple sclerosis; myasthenia gravis; myocardial infarction; neuromyelitis optica; ocular angiogenesis; Parkinson's disease; pneumonia; progressive kidney failure; psoriasis; pulmonary embolisms and infarcts; pulmonary fibrosis; pulmonary vasculitis; renal ischemia; renal ischemia-reperfusion injury; rheumatoid arthritis; schizophrenia; SLE nephritis; smoke injury; stroke; systemic inflammatory response in post-pump syndrome due to cardiopulmonary bypass or renal bypass; systemic lupus erythematosus (SLE); thermal injury; traumatic brain injury; uveitis; vasculitis; wet AMD; paroxysmal nocturnal hemoglobinuria (PNH); and/or xenograft rejection) in a subject in need thereof comprising administering a therapeutically effective amount of a co-formulation as set forth herein to the subject. In an embodiment of the invention, the subject is administered one or more further therapeutic agents, such as, for example, an androgen, an anti-coagulant, an anti-inflammatory drug, an antihypertensive, an immunosuppressive agent, a fibrinolytic agent, a lipid-lowering agent, an anti-CD20 agent, an anti-TNF alpha agent, a C3 inhibitor, an anti-thrombotic agent, a corticosteroid, a non-steroidal anti-inflammatory drug, an angiotensin-converting enzyme inhibitor, an inhibitor of hydroxymethylglutaryl CoA reductase, an anti-seizure agent, warfarin, aspirin, heparin, phenindione, fondaparinux, idraparinux, and thrombin inhibitors such as argatroban, lepirudin, bivalirudin, dabigatran, vincristine, cyclosporine A, methotrexate, ancrod, ε-aminocaproic acid, antiplasmin-a1, prostacyclin, defibrotide, rituximab, infliximab and/or magnesium sulfate.

The present invention provides a method for increasing the stability of RNA, or for reducing beta-hexosaminidase activity, in a composition, comprising the RNA which is conjugated to a ligand that comprises one or more terminal N-Acetylgalactosamine (GalNAc) residues and/or N-acetylglucosamine (GlcNAc) residues; and beta-hexosaminidase comprising (i) adding GalNAc and/or GlcNAc to the composition and/or (ii) increasing or decreasing the pH of the composition from about 6; for example, wherein the composition comprises the RNA, which is a C5 iRNA; an antibody or antigen-binding fragment thereof that was expressed and isolated from a mammalian host cell (e.g., Chinese hamster ovary (CHO) cell) that comprises the beta-hexosaminidase; and, optionally, a buffer; a viscosity reducer; a stabilizer; and a non-ionic surfactant. In an embodiment of the invention, the RNA is a double stranded RNA, optionally comprising an overhang of 1 or 2 nucleotides on one or both ends, for example, wherein the RNA was chemically synthesized.

The present invention includes a method for making a co-formulation including combining the RNAi and the antibody or antigen-binding fragment, and (i) adding GalNAc to the co-formulation and/or (ii) adjusting the pH of the co-formulation to about or below about 6. Co-formulations, which are the product of the method form part of the present invention.

The present invention provides a method for administering, to a subject, an antibody or antigen-binding fragment thereof that binds specifically to C5 (anti-C5) in combination with a C5 iRNA comprising introducing the antibody or fragment and the iRNA into the body of the subject. In an embodiment of the invention, the antibody or fragment and the iRNA are introduced by a subcutaneous injection or intravenous infusion of a co-formulation that comprises both the antibody or fragment and the iRNA; or subcutaneous injections or intravenous infusions of separate formulations that each comprises either the antibody or fragment or the iRNA.

The present invention provides a method for treating or preventing a C5-associated disease or disorder in a subject in need thereof comprising administering to the subject a therapeutically effective amount of an antibody or antigen-binding fragment thereof that binds specifically to C5 in combination with a C5 iRNA which are in a single co-formulation or are in separate formulations. In an embodiment of the invention, the method further includes administering, to the subject, one or more initial intravenous or subcutaneous loading doses of the antibody or antigen-binding fragment and/or the iRNA. For example, in an embodiment of the invention, the method includes administering one or more doses of both (1) about 400 mg of the anti-C5 antibody or antigen-binding fragment; and (2) about 200 mg of the C5 iRNA; e.g., about 400 mg of the anti-C5 antibody or antigen-binding fragment is administered about every 2, 3 or 4 weeks (±3 days); and about 200 mg of the C5 iRNA is administered about every 4 weeks (±3 days). In an embodiment of the invention, the method includes administering (i) about 400 mg of the anti-C5 antibody or antigen-binding fragment subcutaneously about every 2 weeks (±3, 4, 5, 6 or 7 days), and about 200 mg of the C5 iRNA subcutaneously about every 4 weeks (±3, 4, 5, 6 or 7 days); (ii) about 400 mg of the anti-C5 antibody or antigen-binding fragment subcutaneously about every 4 weeks (±3, 4, 5, 6 or 7 days), and about 200 mg of the C5 iRNA subcutaneously about every 4 weeks (±3, 4, 5, 6 or 7 days); (iii) an intravenous loading dose of anti-C5 antibody or antigen-binding fragment, about 400 mg of the anti-C5 antibody or antigen-binding fragment subcutaneously and about 200 mg of the C5 iRNA subcutaneously; and then, about every 4 weeks (±3, 4, 5, 6 or 7 days) thereafter, about 400 mg of the anti-C5 antibody or antigen-binding fragment subcutaneously and about 200 mg of the C5 iRNA subcutaneously; (iv) an intravenous loading dose of about 30 or 60 mg/kg anti-C5 antibody or antigen-binding fragment, about 400 mg of the anti-C5 antibody or antigen-binding fragment subcutaneously and about 200 mg of the C5 iRNA subcutaneously; and then, about every 4 weeks (±3, 4, 5, 6 or 7 days) thereafter, about 400 mg of the anti-C5 antibody or antigen-binding fragment subcutaneously and about 200 mg of the C5 iRNA subcutaneously; (v) an intravenous loading dose of about 30 or 60 mg/kg anti-C5 antibody or antigen-binding fragment followed by one or more weekly subcutaneous doses of about 800 mg anti-C5 antibody or antigen-binding fragment, then, after an optional 1 week period, about 400 mg of the anti-C5 antibody or antigen-binding fragment subcutaneously and about 200 mg of the C5 iRNA subcutaneously; and then, about every 4 weeks (±3, 4, 5, 6 or 7 days) thereafter, about 400 mg of the anti-C5 antibody or antigen-binding fragment subcutaneously and about 200 mg of the C5 iRNA subcutaneously; (vi) (a) a dose of Eculizumab intravenously and about 200 mg C5 iRNA subcutaneously; (b) a dose of the Eculizumab up to about 14 days (±3, 4, 5, 6 or 7 days) later; and (c) about another 14 or 15 days (±3, 4, 5, 6 or 7 days) later, an anti-C5 antibody or antigen-binding fragment dose of 30 or 60 mg/kg body weight intravenously, an anti-C5 antibody or antigen-binding fragment about 400 mg subcutaneously and C5 iRNA about 200 mg subcutaneously and (d) about every 4 weeks (±3, 4, 5, 6 or 7 days) thereafter, a dose of anti-C5 antibody or antigen-binding fragment about 400 mg subcutaneously and C5 iRNA about 200 mg subcutaneously; or (vii) (a) about a 200 mg SC dose of C5 iRNA; (b) about 28 days (±3, 4, 5, 6 or 7 days) later, a 30 or 60 mg/kg IV loading dose of anti-C5 antibody or antigen-binding fragment, a 400 mg SC dose of anti-C5 antibody or antigen-binding fragment and a 200 mg SC dose of C5 iRNA; and (c) about another 29 days (±3, 4, 5, 6 or 7 days) later and about every 4 weeks (±3, 4, 5, 6 or 7 days) thereafter, about a 400 mg SC dose of anti-C5 antibody or antigen-binding fragment and about a 200 mg SC dose of C5 iRNA; or (viii) (a) about 4 weeks (±3, 4, 5, 6 or 7 days) after an administration of Ravulizumab, a 200 mg SC dose of C5 iRNA; (b) about another 28 days (±3, 4, 5, 6 or 7 days) later, a 30 or 60 mg/kg IV loading dose of anti-C5 antibody or antigen-binding fragment, a 400 mg SC dose of anti-C5 antibody or antigen-binding fragment and a 200 mg SC dose of C5 iRNA; and (c) about another 29 days (±3, 4, 5, 6 or 7 days) later and about every 4 weeks (±3, 4, 5, 6 or 7 days) thereafter, a 400 mg SC dose of anti-C5 antibody or antigen-binding fragment and a 200 mg SC dose of C5 iRNA. In an embodiment of the invention, the anti-C5 antibody or antigen-binding fragment and C5 iRNA are administered about every 4 weeks (±3, 4, 5, 6 or 7 days) subcutaneously in a single injection of a co-formulation that comprises the anti-C5 antibody or antigen-binding fragment and C5 iRNA; and about every 4 weeks (±3, 4, 5, 6 or 7 days) a further injection of the anti-C5 antibody or antigen-binding fragment is administered subcutaneously; the anti-C5 antibody or antigen-binding fragment and C5 iRNA are administered about every 4 weeks (±3, 4, 5, 6 or 7 days) subcutaneously in separate injections of separate formulations wherein one comprises the anti-C5 antibody or antigen-binding fragment and the other comprises the C5 iRNA; and about every 4 weeks (±3, 4, 5, 6 or 7 days) a further injection of the anti-C5 antibody or antigen-binding fragment is administered subcutaneously; the anti-C5 antibody or antigen-binding fragment and C5 iRNA are administered about every 4 weeks (±3, 4, 5, 6 or 7 days) subcutaneously in a single injection of a co-formulation that comprises the anti-C5 antibody or antigen-binding fragment and C5 iRNA; and about every 2 weeks (±3, 4, 5, 6 or 7 days) a further injection of the anti-C5 antibody or antigen-binding fragment is administered subcutaneously; and/or the anti-C5 antibody or antigen-binding fragment and C5 iRNA are administered about every 4 weeks (±3, 4, 5, 6 or 7 days) subcutaneously in separate injections of separate formulations wherein one comprises the anti-C5 antibody or antigen-binding fragment and the other comprises the C5 iRNA; and about every 2 weeks (±3, 4, 5, 6 or 7 days) a further injection of the anti-C5 antibody or antigen-binding fragment is administered subcutaneously.

In an embodiment of the invention, the subject has previously received Ravulizumab (e.g., administered intravenous or subcutaneous) and/or Eculizumab (e.g., intravenously administered, e.g., 900 mg intravenously) therapy; and/or Pozelimab monotherapy. In an embodiment of the invention, the subject is complement inhibitor naïve.

The present invention includes a method for treating or preventing a C5-associated disease or disorder in a subject in need thereof comprising administering to the subject a therapeutically effective amount of an anti-C5 antibody or antigen-binding fragment thereof and C5 iRNA, wherein the subject has previously received Eculizumab wherein the subject is administered: (i) a dose of Eculizumab intravenously and 200 mg C5 iRNA subcutaneously; (ii) a dose of the Eculizumab up to about 14 days (±3, 4, 5, 6 or 7 days) later (about day 15); (iii) about 14 or 15 days (±3, 4, 5, 6 or 7 days) later (about day 29), the anti-C5 antibody or antigen-binding fragment at a dose of about 60 mg/kg body weight intravenously, about 400 mg of the anti-C5 antibody or antigen-binding fragment subcutaneously and about 200 mg of the C5 iRNA subcutaneously; and (iv) starting about 28 days (±3, 4, 5, 6 or 7 days) later (about day 57) and about every about 28 days (±3, 4, 5, 6 or 7 days) thereafter, about 400 mg of the anti-C5 antibody or antigen-binding fragment subcutaneously and about 200 mg of the C5 iRNA subcutaneously.

The present invention provides a method for treating or preventing a C5-associated disease or disorder in a subject in need thereof comprising administering to the subject a therapeutically effective amount of an anti-C5 antibody or antigen-binding fragment thereof and a C5 iRNA, wherein the subject has previously received Ravulizumab wherein the subject is administered: (i) about 28 days (±3, 4, 5, 6 or 7 days) after the last administration of Ravulizumab, about a 200 mg SC dose of C5 iRNA; (ii) about 28 days (±3, 4, 5, 6 or 7 days) later (about day 29), about a 60 mg/kg IV dose of anti-C5 antibody or antigen-binding fragment, about a 400 mg SC dose of anti-C5 antibody or antigen-binding fragment and about a 200 mg SC dose of C5 iRNA; (iii) starting about 28 days (±3, 4, 5, 6 or 7 days) later (about day 57) and about every about 28 days (±3, 4, 5, 6 or 7 days) thereafter, about a 400 mg SC dose of anti-C5 antibody or antigen-binding fragment and about a 200 mg SC dose of C5 iRNA.

The present invention provides a method for treating or preventing a C5-associated disease or disorder in a subject in need thereof comprising administering to the subject a therapeutically effective amount of an anti-C5 antibody or antigen-binding fragment thereof and a C5 iRNA, wherein the subject has not previously received complement inhibitor treatment or not received complement inhibitor treatment recently, wherein the subject is administered (i) on about day 1, an intravenous dose of about 30 mg/kg anti-C5 antibody or antigen-binding fragment, about a 400 mg subcutaneous (SC) dose of the antibody or fragment, and about a 200 mg SC dose of the C5 iRNA; and (ii) starting about 28 days later (±3, 4, 5, 6 or 7 days) and about every 4 weeks (±3, 4, 5, 6 or 7 days) thereafter, about 400 mg SC of the anti-C5 antibody or antigen-binding fragment and about 200 mg SC of the C5 iRNA.

The present invention provides a method for treating or preventing a C5-associated disease or disorder in a subject in need thereof comprising administering to the subject a therapeutically effective amount of an anti-C5 antibody or antigen-binding fragment thereof and a C5 iRNA, wherein the subject has previously received anti-C5 antibody or antigen-binding fragment monotherapy (i) starting about 7 to 8 (±3 days) days after the last dose of anti-C5 antibody or antigen-binding fragment monotherapy or when the next dose of the monotherapy is due and about every 4 weeks (±3, 4, 5, 6 or 7 days) thereafter, about a 400 mg SC dose of the anti-C5 antibody or antigen-binding fragment and about a 200 mg SC dose of the C5 iRNA; or (ii) starting about 7 to 8 (±3 days) days after the last dose of anti-C5 antibody or antigen-binding fragment monotherapy or when the next dose of the monotherapy is due: about a 400 mg SC dose of the anti-C5 antibody or antigen-binding fragment and another the dose about every 2 weeks (±3, 4, 5, 6 or 7 days) thereafter; and about a 200 mg SC dose of the C5 iRNA and another the dose about every 4 weeks (±3, 4, 5, 6 or 7 days) thereafter.

The present invention further provides a method for treating or preventing a C5-associated disease or disorder in a subject in need thereof comprising administering to the subject a therapeutically effective amount of an anti-C5 antibody or antigen-binding fragment thereof in combination with a C5 iRNA, wherein the subject has received one or more doses of a non-competing anti-C5 antibody or antigen-binding fragment (N/C Ab) (e.g., wherein the subject has detectable blood levels of N/C Ab when treatment is initiated): (1) a dose of C5 iRNA and the non-competing antibody or fragment on the day the dose of N/C Ab is due; (2) the next dose of non-competing anti-C5 antibody or antigen-binding fragment on the day such dose is due; (3) after about 1-2 half-lives of the N/C Ab, Pozelimab 60 mg/kg IV loading dose, Pozelimab 400 mg SC and Cemdisiran 200 mg SC; (4) starting 4 weeks thereafter, Pozelimab 400 mg SC Q4W and Cemdisiran 200 mg SC Q4W; or (1) following about 1-2 half-lives of the non-competing anti-C5 antibody or antigen-binding fragment from the last dose thereof, a dose of C5 iRNA; (2) following about another 1-2 half-lives of the N/C Ab, Pozelimab 60 mg/kg IV loading dose, Pozelimab 400 mg SC and Cemdisiran 200 mg SC; and (3) starting 4 weeks thereafter, Pozelimab 400 mg SC Q4W and Cemdisiran 200 mg SC Q4W. In an embodiment of the invention, the C5 iRNA is Cemdisiran; the anti-C5 antibody or antigen-binding fragment thereof is Pozelimab; the non-competing anti-C5 antibody or antigen-binding fragment is Eculizumab; the non-competing anti-C5 antibody or antigen-binding fragment is Ravulizumab; the half-life of the non-competing antibody is about 11 days; and/or the half-life of the non-competing antibody is about 32 days.

In an embodiment of the invention, during treatment, the subject achieves or achieves and maintains any one or more of: hemoglobin stabilization; does not receive a red blood cell transfusion; has no decrease in hemoglobin ≥2 g/dL; does not experience breakthrough hemolysis; CH50 levels in blood are fully suppressed relative to baseline (at 0 klU/L) before treatment and/or during any breakthrough hemolysis event; lack of treatment emergent adverse events; Improvement in fatigue, relative to before treatment; >5 point improvement in FACIT-Fatigue score relative to before treatment; improvement in physical functioning score on the European; organization for Research and Treatment of Cancer: Quality-of-Life Questionnaire; core 30 items (EORTC QLQ-C30)) relative to before treatment; improvement in GHS/QoL (global health status/QOL scale (GHS)), relative to before treatment; reduction in lactate dehydrogenase (LDH) levels relative to before treatment; achievement of LDH≤1.5× upper limit of normal (ULN) relative to before treatment achievement and maintenance of LDH≤1.0×ULN; a reduction in blood bilirubin levels relative to before treatment; a reduction in reticulocyte count relative to before treatment; a reduction in alternative pathway hemolytic activity assay (AH50) relative to before treatment; a reduction in PNH erythrocytes and/or granulocytes relative to before treatment; improvement in fatigue, shortness of breath, muscle weakness, headache, abdominal, pain, pain in back/legs, chest discomfort, difficulty sleeping, difficulty thinking clearly, and/or difficulty swallowing relative to before treatment; improvement in renal function as measured by estimated glomerular filtration rate (eGFR) relative to before treatment; reduction in blood free hemoglobin relative to before treatment; reduction in total C5 blood levels relative to before treatment; reduction in PNH clone size relative to before treatment; and/or increase in haptoglobin level relative to before treatment.

In an embodiment of the invention, the C5-associated disease or disorder is a disorder of inappropriate or undesirable complement activation; a hemodialysis complication; a lung disease or disorder; a neurological disorder; a parasitic disease; a post-ischemic reperfusion condition; a proteinuric kidney disease; a renal disorder; adult respiratory distress syndrome (ARDS); age-related macular degeneration (AMD); allergy; Alport's syndrome; Alzheimer's disease; an autoimmune disease; an immune complex disorder; an inflammatory disorder; an ocular disease; an organic dust disease; angiopathic thrombosis and protein-losing enteropathy; asthma; atherosclerosis; bronchoconstriction; bullous pemphigoid; C3 glomerulopathy; capillary leak syndrome; CHAPLE disease (CD55 deficiency with hyperactivation of complement; chemical injury due to irritant gasses and/or chemicals; chronic obstructive pulmonary disease (COPD); complement activation due to burn; complement activation due to frostbite; complement activation due to obesity; complement activation due to sepsis; Crohn's disease; diabetes; diabetic macular edema (DME); diabetic nephropathy; diabetic retinopathy; dry AMD; dyspnea; emphysema; epilepsy; fibrogenic dust diseases; geographic atrophy (GA); glomerulopathy; Goodpasture's Syndrome; Guillain-Barre Syndrome; hemolytic anemia; hemoptysis; hereditary angioedema; hyperacute allograft rejection; hypersensitivity pneumonitis; immune complex-associated inflammation; infectious disease; inflammation of an autoimmune disease; inherited CD59 deficiency; injury due to inert dusts and/or minerals; interleukin-2 induced toxicity during IL-2 therapy; lupus nephritis; membranoproliferative glomerulonephritis; membranoproliferative nephritis; mesenteric artery reperfusion after aortic reconstruction; multiple sclerosis; myasthenia gravis; myocardial infarction; neuromyelitis optica; ocular angiogenesis; Parkinson's disease; pneumonia; progressive kidney failure; psoriasis; pulmonary embolisms and infarcts; pulmonary fibrosis; pulmonary vasculitis; renal ischemia; renal ischemia-reperfusion injury; rheumatoid arthritis; schizophrenia; SLE nephritis; smoke injury; stroke; systemic inflammatory response in post-pump syndrome due to cardiopulmonary bypass or renal bypass; systemic lupus erythematosus (SLE); thermal injury; traumatic brain injury; uveitis; vasculitis; wet AMD; paroxysmal nocturnal hemoglobinuria (PNH); and/or xenograft rejection.

In an embodiment of the invention, the C5 iRNA and the anti-C5 antibody or antigen-binding fragment are co-formulated into a co-formulation and both the antibody or fragment and the C5 iRNA are administered by way of a single injection of the co-formulation.

In an embodiment of the invention, the co-formulation has a pH of about 6.5. In an embodiment of the invention, the C5 iRNA and the anti-C5 antibody or antigen-binding fragment are co-formulated into a co-formulation comprising 100 mg/ml Cemdisiran and 100 mg/ml Pozelimab; or 50 mg/ml Cemdisiran and 100 mg/ml Pozelimab.

In an embodiment of the invention, the co-formulation includes Cemdisiran; Pozelimab that was expressed and isolated from a mammalian host cell that includes beta-hexosaminidase; a buffer; a viscosity reducer; a stabilizer; a non-ionic surfactant and an optional viscosity reducer; at a pH of about 6.5.

In an embodiment of the invention, the subcutaneous injection is performed with a pre-filled syringe or an autoinjector.

In an embodiment of the invention, the subject suffers from aplastic anemia and/or myelodysplastic syndrome.

In an embodiment of the invention, the subject has previously received or which further comprises administering, before (optionally, which is any of 1, 2, 3, 4, 5, 6, 7, 8, 9 10, 11, 12, 13, 14, 15, 16, 17, 18, 19, 20, 21, 22, 23, 24, 25, 26, 27, 28, 29 or 30 days before), after or during the administering of 400 mg subcutaneous Pozelimab and 200 mg subcutaneous Cemdisiran, to the subject: one or more doses of subcutaneous or intravenous Pozelimab; one or more 400 mg subcutaneous doses of Pozelimab; one or more doses of subcutaneous or intravenous anti-C5 antibody or antigen-binding fragment; one or more doses of subcutaneous or intravenous Eculizumab; one or more doses of subcutaneous or intravenous Ravulizumab; one or more doses of subcutaneous or intravenous Cemdisiran; one or more doses of subcutaneous or intravenous C5 iRNA; one or more subcutaneous doses of 800 mg Pozelimab; one or more subcutaneous doses of 800 mg anti-C5 antibody or antigen-binding fragment; one or more intravenous doses of 30 mg/kg body weight Pozelimab; one or more intravenous doses of 30 mg/kg body weight anti-C5 antibody or antigen-binding fragment; one or more intravenous doses of about 60 mg/kg body weight of Pozelimab; one or more intravenous doses of about 60 mg/kg body weight of anti-C5 antibody or antigen-binding fragment; one or more subcutaneous doses of about 800 mg of Pozelimab; one or more subcutaneous doses of about 800 mg of anti-C5 antibody or antigen-binding fragment; one intravenous dose of about 60 mg/kg body weight of Pozelimab and then one or more subcutaneous doses of about 800 mg of Pozelimab; one intravenous dose of about 60 mg/kg body weight of anti-C5 antibody or antigen-binding fragment and then one or more subcutaneous doses of about 800 mg of anti-C5 antibody or antigen-binding fragment; one or more doses of ≥300, ≥600, ≥900 or 1200 mg of Eculizumab intravenously; one or more doses of 200 mg of Cemdisiran subcutaneously; and/or one or more doses of 200 mg of C5 iRNA subcutaneously.

In an embodiment of the invention, intravenous administration of anti-C5 antibody or antigen-binding fragment is separated from subcutaneous administration of anti-C5 antibody or antigen-binding fragment or C5 iRNA by about 30 minutes; subcutaneous administration of anti-C5 antibody or antigen-binding fragment and C5 iRNA is followed by an observation period of about 30 minutes, 1 hour or 2 hours; and/or subcutaneous administration of C5 iRNA is followed by an observation period of about 30 minutes, 1 hour or 2 hours.

In an embodiment of the invention, if the subject exhibits one or more of the criteria: breakthrough hemolysis that is not due to a complement activating condition; and/or LDH increase ≥2×ULN due to a complement activating condition, then the subject receives an intensified treatment further comprising one or more 30 mg/kg IV doses of anti-C5 antibody or antigen-binding fragment.

In an embodiment of the invention the subject exhibits one or more of the criteria: breakthrough hemolysis that is not due to a complement activating condition; and/or LDH increase ≥2×ULN due to a complement activating condition, then the subject receives an intensified treatment wherein: (1) if the subject had received a treatment regimen comprising about 400 mg of the anti-C5 antibody or antigen-binding fragment administered subcutaneously about every 4 weeks (±3, 4, 5, 6 or 7 days), and about 200 mg of the C5 iRNA administered subcutaneously about every 4 weeks (±3, 4, 5, 6 or 7 days); then administering a single 30 mg/kg IV dose of anti-C5 antibody or antigen-binding fragment on the day of intensification and an intensified regimen of about 400 mg of the anti-C5 antibody or antigen-binding fragment administered subcutaneously about every 2 weeks (±3, 4, 5, 6 or 7 days), and about 200 mg of the C5 iRNA administered subcutaneously about every 4 weeks (±3, 4, 5, 6 or 7 days) is administered starting on the day of intensification; or (2) if the subject had received a treatment regimen comprising about 400 mg of the anti-C5 antibody or antigen-binding fragment administered subcutaneously about every 2 weeks (±3, 4, 5, 6 or 7 days), and about 200 mg of the C5 iRNA administered subcutaneously about every 4 weeks (±3, 4, 5, 6 or 7 days); then administering a single 30 mg/kg IV dose of anti-C5 antibody or antigen-binding fragment on the day of intensification and re-initiation of the treatment regimen comprising about 400 mg of the anti-C5 antibody or antigen-binding fragment administered subcutaneously about every 2 weeks (±3, 4, 5, 6 or 7 days), and about 200 mg of the C5 iRNA administered subcutaneously about every 4 weeks (±3, 4, 5, 6 or 7 days) starting on the day of intensification.

In an embodiment of the invention, the anti-C5 antibody or antigen-binding fragment or Pozelimab is expressed in a mammalian host cell (e.g., Chinese hamster ovary cell) and the iRNA or Cemdisiran is chemically synthesized.

In an embodiment of the invention, the anti-C5 antibody or antigen-binding fragment and C5 iRNA are co-formulated into a co-formulation that comprises no more than about 2.1 parts per million (ppm) molar ratio of beta-hexosaminidase to antibody or antigen-binding fragment; include no more than about 0.170 micrograms/ml beta-hexosaminidase, include no more than about 0.04 micrograms/ml beta-hexosaminidase; and/or about 0.04; 0.05; 0.06; 0.0605; 0.063; 0.07; 0.0765; 0.078; 0.08; 0.14; 0.141; 0.15; 0.1525; 0.166; or 0.17 micrograms/ml beta-hexosaminidase; or no more than any of such concentrations.

In an embodiment of the invention, the anti-C5 antibody or antigen-binding fragment thereof is (1) a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10; (2) a heavy chain variable region (HCVR) that comprises the HCDR1, HCDR2 and HCDR3 of a HCVR that comprises the amino acid sequence set forth in SEQ ID NO: 18, and a light chain variable region (LCVR) that comprises the LCDR1, LCDR2 and LCDR3 of a LCVR that comprises the amino acid sequence set forth in SEQ ID NO: 26; (3) a heavy chain variable region (HCVR) that comprises the HCDR1, HCDR2 and HCDR3 of a HCVR that comprises the amino acid sequence set forth in SEQ ID NO: 34, and a light chain variable region (LCVR) that comprises the LCDR1, LCDR2 and LCDR3 of a LCVR that comprises the amino acid sequence set forth in SEQ ID NO: 42; (4) a heavy chain variable region (HCVR) that comprises the HCDR1, HCDR2 and HCDR3 of a HCVR that comprises the amino acid sequence set forth in SEQ ID NO: 50, and a light chain variable region (LCVR) that comprises the LCDR1, LCDR2 and LCDR3 of a LCVR that comprises the amino acid sequence set forth in SEQ ID NO: 58; (5) a heavy chain variable region (HCVR) that comprises the HCDR1, HCDR2 and HCDR3 of a HCVR that comprises the amino acid sequence set forth in SEQ ID NO: 66, and a light chain variable region (LCVR) that comprises the LCDR1, LCDR2 and LCDR3 of a LCVR that comprises the amino acid sequence set forth in SEQ ID NO: 74; (6) a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90; (7) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 106; (8) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 114; (9) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 106; (10) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 130; (11) a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 106; (12) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 106; (13) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130; (14) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 114; (15) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 130; (16) a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 130; (17) a heavy chain variable region (HCVR) that comprises the HCDR1, HCDR2 and HCDR3 of a HCVR that comprises the amino acid sequence set forth in SEQ ID NO: 154, and a light chain variable region (LCVR) that comprises the LCDR1, LCDR2 and LCDR3 of a LCVR that comprises the amino acid sequence set forth in SEQ ID NO: 162; (18) a heavy chain variable region (HCVR) that comprises the HCDR1, HCDR2 and HCDR3 of a HCVR that comprises the amino acid sequence set forth in SEQ ID NO: 170, and a light chain variable region (LCVR) that comprises the LCDR1, LCDR2 and LCDR3 of a LCVR that comprises the amino acid sequence set forth in SEQ ID NO: 178; (19) a heavy chain variable region (HCVR) that comprises the HCDR1, HCDR2 and HCDR3 of a HCVR that comprises the amino acid sequence set forth in SEQ ID NO: 186, and a light chain variable region (LCVR) that comprises the LCDR1, LCDR2 and LCDR3 of a LCVR that comprises the amino acid sequence set forth in SEQ ID NO: 194; (20) a heavy chain variable region (HCVR) that comprises the HCDR1, HCDR2 and HCDR3 of a HCVR that comprises the amino acid sequence set forth in SEQ ID NO: 202, and a light chain variable region (LCVR) that comprises the LCDR1, LCDR2 and LCDR3 of a LCVR that comprises the amino acid sequence set forth in SEQ ID NO: 210; (21) a heavy chain variable region (HCVR) that comprises the HCDR1, HCDR2 and HCDR3 of a HCVR that comprises the amino acid sequence set forth in SEQ ID NO: 218, and a light chain variable region (LCVR) that comprises the LCDR1, LCDR2 and LCDR3 of a LCVR that comprises the amino acid sequence set forth in SEQ ID NO: 226; (22) a heavy chain variable region (HCVR) that comprises the HCDR1, HCDR2 and HCDR3 of a HCVR that comprises the amino acid sequence set forth in SEQ ID NO: 234, and a light chain variable region (LCVR) that comprises the LCDR1, LCDR2 and LCDR3 of a LCVR that comprises the amino acid sequence set forth in SEQ ID NO: 242; (23) a heavy chain variable region (HCVR) that comprises the HCDR1, HCDR2 and HCDR3 of a HCVR that comprises the amino acid sequence set forth in SEQ ID NO: 250, and a light chain variable region (LCVR) that comprises the LCDR1, LCDR2 and LCDR3 of a LCVR that comprises the amino acid sequence set forth in SEQ ID NO: 258; (24) a heavy chain variable region (HCVR) that comprises the HCDR1, HCDR2 and HCDR3 of a HCVR that comprises the amino acid sequence set forth in SEQ ID NO: 266, and a light chain variable region (LCVR) that comprises the LCDR1, LCDR2 and LCDR3 of a LCVR that comprises the amino acid sequence set forth in SEQ ID NO: 258; (25) a heavy chain variable region (HCVR) that comprises the HCDR1, HCDR2 and HCDR3 of a HCVR that comprises the amino acid sequence set forth in SEQ ID NO: 274, and a light chain variable region (LCVR) that comprises the LCDR1, LCDR2 and LCDR3 of a LCVR that comprises the amino acid sequence set forth in SEQ ID NO: 282; (26) a heavy chain variable region (HCVR) that comprises the HCDR1, HCDR2 and HCDR3 of a HCVR that comprises the amino acid sequence set forth in SEQ ID NO: 290, and a light chain variable region (LCVR) that comprises the LCDR1, LCDR2 and LCDR3 of a LCVR that comprises the amino acid sequence set forth in SEQ ID NO: 298; (27) a heavy chain variable region (HCVR) that comprises the HCDR1, HCDR2 and HCDR3 of a HCVR that comprises the amino acid sequence set forth in SEQ ID NO: 306, and a light chain variable region (LCVR) that comprises the LCDR1, LCDR2 and LCDR3 of a LCVR that comprises the amino acid sequence set forth in SEQ ID NO: 314; (28) a heavy chain variable region (HCVR) that comprises the HCDR1, HCDR2 and HCDR3 of a HCVR that comprises the amino acid sequence set forth in SEQ ID NO: 322, and a light chain variable region (LCVR) that comprises the LCDR1, LCDR2 and LCDR3 of a LCVR that comprises the amino acid sequence set forth in SEQ ID NO: 330; and/or (29) a heavy chain variable region (HCVR) that comprises the HCDR1, HCDR2 and HCDR3 of a HCVR that comprises the amino acid sequence set forth in SEQ ID NO: 338, and a light chain variable region (LCVR) that comprises the LCDR1, LCDR2 and LCDR3 of a LCVR that comprises the amino acid sequence set forth in SEQ ID NO: 346.

In an embodiment of the invention, the C5 iRNA comprises an RNA strand that is complementary to an mRNA transcribed from the C5 gene sense strand DNA sequence AAGCAAGATATTTTTATAATA (nucleotides 782-802 of SEQ ID NO: 360). In an embodiment of the invention, the C5 iRNA is a double-stranded ribonucleic acid (dsRNA) agent comprising a sense strand and an antisense strand, wherein the antisense strand comprises a region of complementarity comprising at least 17 contiguous nucleotides differing by no more than 3 nucleotides from the nucleotide sequence of 5′-UAUUAUAAAAAUAUCUUGCUUUU-3′ (SEQ ID NO: 364), and wherein the dsRNA agent comprises at least one modified nucleotide. In an embodiment of the invention the C5 iRNA is a double-stranded ribonucleic acid (dsRNA) agent comprising a sense strand and an antisense strand, wherein the sense strand comprises 5′-asasGfcAfaGfaUfAfUfuUfuuAfuAfaua-3′ (SEQ ID NO: 406) and the antisense strand comprises 5′-usAfsUfuAfuaAfaAfauaUfcUfuGfcuususudTdT-3′ (SEQ ID NO: 369), wherein a, g, c and u are 2′-O-methyl (2′-OMe) A, G, C, and U, respectively; Af, Gf, Cf and Uf are 2′-fluoro A, G, C and U, respectively; dT is a deoxy-thymine nucleotide; s is a phosphorothioate linkage; and wherein the sense strand is conjugated at the 3′-terminus to the ligand

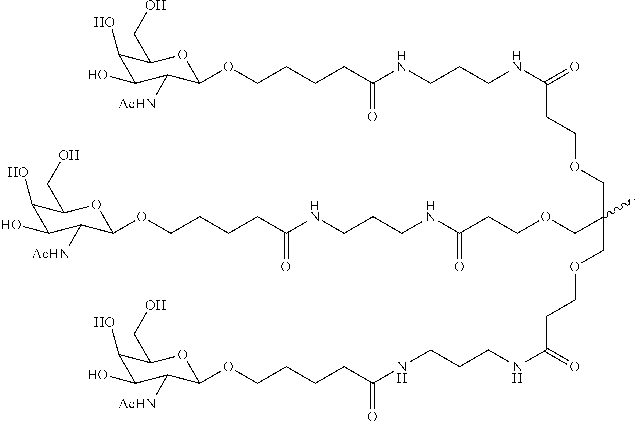 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In an embodiment of the invention, the C5 iRNA and the antibody or antigen-binding fragment thereof that binds specifically to C5 are in a co-formulation that is specifically set forth herein.

In an embodiment of the invention, wherein the C5 iRNA and the anti-C5 antibody or antigen-binding fragment thereof are in a single co-formulation which, when administered subcutaneously, is administered in 1 or 2 or more (e.g., 2) injections of said co-formulation.

Preferably, the C5 iRNA is Cemdisiran; and/or the anti-C5 antibody or antigen-binding fragment thereof is Pozelimab.

SUMMARY: Complement inhibitor naïve: On day 1: Pozelimab in a single loading dose of 30 mg/kg intravenous (IV) and 400 mg subcutaneous (SC), and Cemdisiran 200 mg SC (the combination maintenance dose); Starting on day 29: Pozelimab 400 mg SC every 4 weeks (q4W) and Cemdisiran 200 mg SC q4W (e.g., in a Cemdisiran/Pozelimab co-formulation). Switch from Pozelimab monotherapy to Pozelimab+Cemdisiran combination therapy: the last dose of Pozelimab monotherapy) or, when the next dose of Pozelimab monotherapy is due, subjects start receiving Pozelimab 400 mg SC every 4 weeks (q4W) and Cemdisiran 200 mg SC q4W; Switch from Eculizumab therapy to Pozelimab+Cemdisiran combination therapy: On day 1 (the day of subject's scheduled Eculizumab administration): Cemdisiran 200 mg SC and Eculizumab >900 mg IV (subject's usual dose); On day 15, for subjects on Eculizumab q14 days (labeled dose regimen): Labeled Eculizumab dose [for subjects on Eculizumab more frequently than q14 days: patients are dosed within 2 days of their usual planned dose; On day 29 (or when the next Eculizumab is due (if on Eculizumab doses more frequent than q14w) or 2 weeks later): Pozelimab 60 mg/kg IV loading dose, and Pozelimab 400 mg SC and Cemdisiran 200 mg SC; and Starting on day 57 (or 4 weeks later): Pozelimab 400 mg SC and Cemdisiran 200 mg SC q4W; Switch from Ravulizumab therapy to Pozelimab+Cemdisiran combination therapy: On day 1 (4 weeks after the last administration of Ravulizumab): Cemdisiran 200 mg SC; On day 29 or 4 weeks later: Pozelimab 60 mg/kg single IV loading dose, and Pozelimab 400 mg SC and Cemdisiran 200 mg SC; and Starting on Day 57 or 4 weeks later: Start Pozelimab 400 mg SC q4W and Cemdisiran 200 mg SC q4W.

### BRIEF DESCRIPTION OF THE FIGURES

FIG. 1: Cemdisiran structure. Duplex RNA sense strand and anti-sense strand with modified nucleotides having sense strand linked to a ligand (L96).

FIG. 2: Stability of Cemdisiran (total impurities #1 and #2) over time at 5° C. of 75:100 and 100:100 co-formulations (Cemdisiran:Pozelimab concentration (mg/ml)).

FIG. 3: Stability of Cemdisiran (total impurities #1 and #2) over time at 40° C. of 75:100 and 100:100 co-formulations (Cemdisiran:Pozelimab concentration (mg/ml)) along with Cemdisiran only control.

FIG. 4: Chromatograms from dIPRP analysis of 100:100, 75:100 (Cemdisiran:Pozelimab concentration (mg/ml)) and Cemdisiran only samples stored for 3 months at 40° C.

FIG. 5: Structure representing Cemdisiran impurity 1 lacking one GalNAc (wavy line represents double stranded RNA).

FIG. 6: Cemdisiran purity (by dIPRP) for co-formulations 75:100 and 100:100 (Cemdisiran:Pozelimab concentration (mg/ml)) manufactured from Pozelimab process 1 and 2 material and stored at 40° C.

FIG. 7: Chromatograms from dIPRP analysis after 0.5 month at 40° C. of two Cemdisiran only formulations (±10 micrograms/ml beta-hexosaminidase).

FIG. 8: Total impurity (Cemdisiran impurities #1, #2 and #3) over time in storage at all three temperatures (left to right: 40° C., 25° C. and 5° C.) between the two 50:100 co-formulations (Cemdisiran:Pozelimab concentration (mg/ml)) at pH5.9 or pH 6.6.

FIG. 9: Total impurity (Cemdisiran impurities #1, #2 and #3) over time in storage at 40° C. between the two 100:100 and two 50:100 co-formulations (Cemdisiran:Pozelimab concentration (mg/ml)) at pH6.0 made from Pozelimab lot 3 or 4. Degradation progress fit to curves having shown equations.

FIG. 10: Characteristics varied in 50:100 co-formulation evaluated in the DOE (design of experiment) experiments (pH, sucrose, Arginine, Pozelimab (REGN3918), Cemdisiran, Histidine; as well as desirability.

FIG. 11: Characteristics varied in 100:100 co-formulation evaluated in the DOE (design of experiment) experiments (pH, sucrose, Arginine, Pozelimab (REGN3918), Cemdisiran, Histidine; as well as desirability.

FIG. 12: Percent change in high molecular weight species of Pozelimab after agitation at various concentrations of polysorbate 80 with two co-formulations (100:100 and 50:100) (Cemdisiran:Pozelimab concentration (mg/ml)).

FIG. 13: Total impurity (Cemdisiran impurities #1, #2 and #3) over time in storage at 40° C. of two co-formulations at pH6.0 (50:100 and 100:100) and two co-formulations at pH 6.5 (50:100 and 100:100) (Cemdisiran:Pozelimab concentration (mg/ml)).

FIG. 14: Quantitation of beta-hex in various lots of Pozelimab (ng/ml).

FIG. 15: Assay calibration curve.

FIG. 16: Dilution curve.

FIG. 17: Schematic showing Pozelimab+Cemdisiran Dosing regimen for patients previously on Pozelimab monotherapy, as described in Example 4.

FIG. 18: Graph showing individual LDH (×ULN) values over time for patients in arm 1 (Pozelimab Q4W+Cemdisiran) of the study described in Example 4.

FIG. 19: Graph showing individual LDH (×ULN) values over time for patients in arm 2 (Pozelimab Q2W+Cemdisiran) of the study described in Example 4.

FIG. 20: Graphs showing individual hemoglobin values over time for patients in arm 1 (Pozelimab Q4W+Cemdisiran) and arm 2 (Pozelimab Q2W+Cemdisiran) of the study described in Example 4. Each line represents an individual patient.

FIGS. 21A-C are graphs showing patient-reported outcomes over time for patients in the study described in Example 4. FIG. 21A is a graph showing FACIT-Fatigue score, FIG. 21B is a graph showing EORTC-QLQ-C30 physical functioning score, and FIG. 21C is a graph showing EORTC-QLQ-C30 GHS/QoL score.

FIG. 22: Graph showing individual LDH (×ULN) values by visit for patients in the study described in Example 5. Each line represents an individual patient.

FIG. 23: Individual patient LDH values by visit for patients in the study described in Example 5.

FIG. 24: Individual patient hemoglobin values by visit for patients in the study described in Example 5.

FIG. 25: Study Flow Diagram for the study described in Example 5.

FIG. 26: Study Flow Diagram for the study described in Example 6.

FIG. 27: Study Flow Diagram for the study described in Example 7.

FIG. 28: Spaghetti Plot: Ratio of LDH to ULN (LDH/ULN) Results by Visit (Full Analysis Set), Pozelimab q2w+Cemdisiran q4w and Pozelimab q4w+Cemdisiran q4w

FIG. 29: Spaghetti Plot: CH50 Results by Visit (Full Analysis Set), Pozelimab q2w+Cemdisiran q4w and Pozelimab q4w+Cemdisiran q4w

FIG. 30: Spaghetti Plot: LDH (xULN) Results by Visit from Baseline Visit 2 (Day 1) to Day 225 (Full Analysis Set), 1.5×ULN and 1×ULN indicated

FIG. 31: Individual LDH values by visit (5 patients completed the OLTP). Each line represents an individual patient. LDH, lactate dehydrogenase; ULN, upper limit of normal.

FIG. 32: Individual hemoglobin values by visit (5 patients completed the OLTP). Each line represents an individual patient.

FIG. 33: Percentage of patients with LDH≤1.5×ULN over time (at data cut-off, all 24 randomized patients completed the OLTP, and 23 entered the optional OLEP). Arm 1: Pozelimab 400 mg SC Q4W+cemdisiran 200 mg SC Q4W. Arm 2: Pozelimab 400 mg SC Q2W+cemdisiran 200 mg SC Q4W. LDH, lactate dehydrogenase; Q2W, every 2 weeks; Q4W, every 4 weeks; SC, subcutaneous; ULN, upper limit of normal.

FIG. 34: Hemoglobin over time (at data cut-off, all 24 randomized patients completed the OLTP, and 23 entered the optional OLEP). Arm 1: Pozelimab 400 mg SC Q4W+cemdisiran 200 mg SC Q4W. Arm 2: Pozelimab 400 mg SC Q2W+cemdisiran 200 mg SC Q4W. SC, subcutaneous; SE, standard error; Q2W, every 2 weeks; Q4W, every 4 weeks.

FIG. 35: Mean percentage change in lactate dehydrogenase excretion rate from baseline (U/L) over time (by visit (weeks)) among patients (pozelimab 400 mg SC Q4W+cemdisiran 200 mg SC Q4W & ravulizumab patients).

FIG. 36: Spaghetti plot of LDH/ULN results by visit among patients (pozelimab 400 mg SC Q4W+cemdisiran 200 mg SC Q4W & ravulizumab patients)−1.5 and 1×ULN levels indicated. Doses of combination or ravulizumab are indicated.

FIG. 37: Spaghetti plot of LDH/ULN results by visit among five patients that failed to achieve adequate control of LDH by week 8 (pozelimab 400 mg SC Q4W+cemdisiran 200 mg SC Q4W & ravulizumab patients)−1.5 and 1×ULN levels indicated. Doses of combination or ravulizumab are indicated.

FIG. 38: Spaghetti plot of CH50 (U/ml) over time by visit among patients (pozelimab 400 mg SC Q4W+cemdisiran 200 mg SC Q4W & ravulizumab patients). Doses of combination or ravulizumab and CH50 measurements are indicated.

FIG. 39: Spaghetti plot of CH50 (U/ml) over time by visit among patients who were inadequate responders (pozelimab 400 mg SC Q4W+cemdisiran 200 mg SC Q4W & ravulizumab patients). Patient that transitioned to trial R3918-PNH-2050 indicated.

FIG. 40: LDH over time of an inadequate responder in Ravulizumab arm before and after switch to combination.

FIG. 41: On Study Per Protocol Transfusions Between Groups. One patient in each arm that met protocol definition for transfusion but did not receive a transfusion.

FIG. 42: Spaghetti plot of red blood cell hemoglobin (g/i) by visit in patients who completed week 26. Hb=hemoglobin; reference range (g/L): 110 to 155 female, 125 to 170 male

FIG. 43: Spaghetti plots of LDH (xULN) by visit for patients with aplastic anemia (AA) or myelodysplastic syndrome (MDS) reported in medical history. Solid lines represent AA patients and dotted lines represent MDS patients.

FIG. 44: Chemical Structures of Tested Viscosity Reducers. Effect on viscosity in 1:1 base formulation:120 mg/mL Cemdisiran, 120 mg/mL Pozelimab, 15 mM histidine, pH 6.2 and 1:2 base formulation: 75 mg/mL Cemdisiran, 150 mg/mL Pozelimab, 15 mM histidine, pH 6.2 relative to control formulation lacking viscosity reducer shown in parentheses.

FIG. 45: Percentage of Patients with <1.5×ULN by Visit (Cohort A)

FIG. 46: Percentage of Patients with <1.0×ULN by Visit (Cohort A)

FIG. 47: Summary of Cohorts in Study presented in Example 7

FIG. 48: Spaghetti Plot of Red Blood Cell Hemoglobin (g/i) Results by Visit for Subjects that Completed the Open Label Treatment Period (OLTP, Visit Week 26)-Analysis of Cohort A.

FIG. 49: Correspondence of LDH & CH50 in inadequate LDH responders in the combination vs ravulizumab arms. Combo Patient ID: 158-007-102; Ravulizumab Patient IDs:124-001-103; 158-001-101; 410-001-102; and 410-001-104.

FIG. 50: Complement inhibitor naïve, Eculizumab switch, Ravulizumab switch and Pozelimab monotherapy switch regimens. Cem=Cemdisiran; Ecu=Eculizumab; Pz=Pozelimab; SC=subcutaneous; IV=intravenous. In an embodiment of the invention, each regimen is characterized by the timelines shown.

### DETAILED DESCRIPTION

While combining two C5-inhibitor therapies with complementary mechanisms of action (Pozelimab and Cemdisiran) offers the advantages of fully suppressing the C5 pathway at a (relatively) low level of C5 expression, various technical issue must be overcome in order to reach a suitable dosing regimen and delivery vehicle for the agents.

The superior C5 suppression offered by the compositions and methods of the present disclosure results in the need for less Pozelimab, which, in turn, leads to a reduced SC volume of antibody injection, a reduced dosing frequency, a window of drug administration that is wider for the combination than the Pozelimab monotherapy, and the potential for reduced injection site reactions. In patients requiring chronic and long-term administration, the combination offers the potential for improved compliance and quality of life compared to Pozelimab monotherapy, while still providing for maximal inhibition of C5 activity in a greater percentage of patients than Eculizumab therapy. Moreover, as discussed herein, the dosing regimens of the present disclosure avoid the danger of adverse events caused by the formation of large drug-target-drug complexes (e.g., Eculizumab-C5-Pozelimab). The co-formulation of both agents also offers the convenience of only a single subcutaneous injection in order to administer both agents together.

The present disclosure provides a stable co-formulation that comprises an antibody or antigen-binding fragment thereof, e.g., Pozelimab and a C5 iRNA, e.g., Cemdisiran. Co-formulating an antibody expressed from a mammalian host cell and an iRNA molecule conjugated to ligand having a terminal N-acetylgalactosamine (GalNAc) and/or N-acetylglucosamine (GlcNAc) presents technical challenges. Small amounts of an enzyme from such host cells which frequently contaminates antibodies preparations, beta hexosaminidase, has been shown to catalyze the removal of terminal GalNAc residues from such iRNA ligands. The present disclosure provides stable co-formulations that include such antibodies and iRNA molecules which overcome this issue, for example, by adjustment of pH from 6 (e.g., 6.5), the addition of GalNAc and/or GlcNAc; and/or the addition of arginine (e.g., L-arginine such as L-arginine HCl).

The administration of anti-C5 antibody and C5 iRNA in the methods of the present disclosure have been designed to rapidly and continuously suppress concentrations of C5 to pharmacologically inactive levels. Typically, anti-C5 monotherapies call for relative high doses in patients with PNH. The requirement for such high anti-C5 mAb doses is driven by 2 factors. First, C5 levels are high and there is a need for 100% inhibition which can only be achieved with complete target engagement (Peffault de Latour R et al., Assessing complement blockade in patients with paroxysmal nocturnal hemoglobinuria receiving eculizumab. Blood 2015; 125(5):775-83); second, in order to achieve 100% inhibition on a population basis, inter- and intra-patient variability of C5 concentrations and instances of enhanced complement activation (which may occur with intercurrent illness) could be employed. Combinations of Pozelimab and Cemdisiran achieve high complement inhibition with a relatively lower dose of the antibody. Combining Pozelimab and Cemdisiran also offers the advantages achieving the low complement levels while administering less Pozelimab (relative to Pozelimab monotherapy), thereby leading to a reduced SC volume of injection, reduced dosing frequency, a window of drug administration that is wider than that of Pozelimab monotherapy, and the potential for reduced injection site reactions.

The present disclosure includes dosing regimens for switching from a prior anti-C5 antibody therapy (e.g., Eculizumab or Ravulizumab) to a C5 iRNA+anti-C5 antibody or antigen-binding fragment thereof therapy of the present disclosure (e.g., Pozelimab+Cemdisiran). Pozelimab has been shown to bind C5 non-competitively with antibodies having the amino acid sequence of Eculizumab (Eculizumab*), and thus has the potential to form heteromeric complexes including large DTD immune complexes, for example, in patients switching from Eculizumab to Pozelimab therapy. Large DTD immune complexes are known to cause adverse events such as serum sickness-like reactions, skin rash. The dosing regimens of the present disclosure have been designed to reduce the danger of the formation of the large DTD complexes and the occurrence of such adverse events.

### Antigen-Binding Proteins

The term “antibody”, as used herein, refers to immunoglobulin molecules comprising four polypeptide chains, two heavy chains (HCs) and two light chains (LCs), inter-connected by disulfide bonds (e.g., IgG)—for example H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; crovalimab; Eculizumab, tesidolumab, mubodina or Ravulizumab; preferably, Pozelimab. In an embodiment of the disclosure, each antibody heavy chain (HC) comprises a heavy chain variable region (“HCVR” or “V_{H}”) (e.g., SEQ ID NO: 2; 18; 34; 50; 66; 82; 98; 98; 122; 98; 138; 146; 122; 146; 146; 138; 154; 170; 186; 202; 218; 234; 250; 266; 274; 290; 306; 322; or 338; or a variant thereof) and a heavy chain constant region; and each antibody light chain (LC) comprises a light chain variable region (“LCVR or “V_{L}”) (e.g., SEQ ID NO: 10; 26; 42; 58; 74; 90; 106; 114; 106; 130; 106; 106; 130; 114; 130; 130; 162; 178; 194; 210; 226; 242; 258; 258; 282; 298; 314; 330; or 346; or a variant thereof) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. Preferably an antibody or antigen-binding fragment thereof in a co-formulation of the present disclosure was expressed and isolated from a mammalian host cell such as a Chinese hamster ovary (CHO) cell.

Antibodies as set forth herein include, for example, monoclonal, recombinant, chimeric, human and/or humanized antibodies.

In an embodiment of the disclosure, the assignment of amino acids to each framework or CDR domain is in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883. Thus, the present disclosure includes antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined according to Kabat and/or Chothia.

In an embodiment of the disclosure, an anti-C5 antigen-binding protein, e.g., antibody or antigen-binding fragment, comprises a heavy chain constant domain, e.g., of the type IgA (e.g., IgA1 or IgA2), IgD, IgE, IgG (e.g., IgG1, IgG2, IgG3 and IgG4 (e.g., comprising a S228P and/or S108P mutation)) or IgM. In an embodiment of the disclosure, an antigen-binding protein, e.g., antibody or antigen-binding fragment, comprises a light chain constant domain, e.g., of the type kappa or lambda. The present disclosure includes antigen-binding proteins comprising the variable domains set forth herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; crovalimab; Eculizumab, tesidolumab, mubodina or Ravulizumab; preferably, Pozelimab) which are linked to a heavy and/or light chain constant domain, e.g., as set forth above.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

In an embodiment of the disclosure, an antibody or antigen-binding fragment thereof that binds specifically to complement factor 5 (C5) protein, interacts with one or more amino acids contained within NMATGMDSW (SEQ ID NO: 353) (or at least 1, 2, 3, 4 or 5 amino acids therein); or WEVHLVPRRKQLQFALPDSL (SEQ ID NO: 354) (or at least 1, 2, 3, 4 or 5 amino acids therein), as determined by hydrogen/deuterium exchange. In an embodiment of the disclosure, an antibody or antigen-binding fragment thereof that binds specifically to complement factor 5 (C5) protein interacts with one or more amino acids contained within the alpha chain and/or the beta chain of C5, as determined by hydrogen/deuterium exchange. For example, in an embodiment of the disclosure, the antibody or antigen-binding fragment does not interact with an amino acid of the C5a anaphylatoxin region of C5, as determined by hydrogen/deuterium exchange. In an embodiment of the disclosure, an antibody or antigen-binding fragment thereof that binds specifically to complement factor 5 (C5) protein interacts with an amino acid sequence selected from the group consisting of

(SEQ ID NO: 353)
(a) NMATGMDSW;
(SEQ ID NO: 355)
(b) ATGMDSW;
(SEQ ID NO: 356)
(C) WEVHLVPRRKQLQ;
(SEQ ID NO: 354)
(d) WEVHLVPRRKQLQFALPDSL;
and
(SEQ ID NO: 357)
(e) LVPRRKQLQ.

The sequence of anti-C5 antibodies and antigen-binding fragments thereof (e.g., LCVRs and HCVRs or LCDRs and HCDRs thereof) that may be included in a co-formulation or used in a method are set forth below.

TABLE A
Anti-C5 Antibody Chain Amino Acid Sequences*

| Antibody | SEQ ID NOs | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 2 | 4 | 6 | 8 | 10 | 12 | 14 | 16 |
| H2M11686N | 18 | 20 | 22 | 24 | 26 | 28 | 30 | 32 |
| H4H12159P | 34 | 36 | 38 | 40 | 42 | 44 | 46 | 48 |
| H4H12161P | 50 | 52 | 54 | 56 | 58 | 60 | 62 | 64 |
| H4H12163P | 66 | 68 | 70 | 72 | 74 | 76 | 78 | 80 |
| H4H12164P | 82 | 84 | 86 | 88 | 90 | 92 | 94 | 96 |
| H4H12166P | 98 | 100 | 102 | 104 | 106 | 108 | 110 | 112 |
| H4H12166P2 | 98 | 100 | 102 | 104 | 114 | 116 | 118 | 120 |
| H4H12166P3 | 122 | 124 | 126 | 128 | 106 | 108 | 110 | 112 |
| H4H12166P4 | 98 | 100 | 102 | 104 | 130 | 132 | 134 | 136 |
| H4H12166P5 | 138 | 140 | 142 | 144 | 106 | 108 | 110 | 112 |
| H4H12166P6 | 146 | 148 | 150 | 152 | 106 | 108 | 110 | 112 |
| H4H12166P7 | 122 | 124 | 126 | 128 | 130 | 132 | 134 | 136 |
| H4H12166P8 | 146 | 148 | 150 | 152 | 114 | 116 | 118 | 120 |
| H4H12166P9 | 146 | 148 | 150 | 152 | 130 | 132 | 134 | 136 |
| H4H12166P10 | 138 | 140 | 142 | 144 | 130 | 132 | 134 | 136 |
| H4H12167P | 154 | 156 | 158 | 160 | 162 | 164 | 166 | 168 |
| H4H12168P | 170 | 172 | 174 | 176 | 178 | 180 | 182 | 184 |
| H4H12169P | 186 | 188 | 190 | 192 | 194 | 196 | 198 | 200 |
| H4H12170P | 202 | 204 | 206 | 208 | 210 | 212 | 214 | 216 |
| H4H12171P | 218 | 220 | 222 | 224 | 226 | 228 | 230 | 232 |
| H4H12175P | 234 | 236 | 238 | 240 | 242 | 244 | 246 | 248 |
| H4H12176P2 | 250 | 252 | 254 | 256 | 258 | 260 | 262 | 264 |
| H4H12177P2 | 266 | 268 | 270 | 272 | 258 | 260 | 262 | 264 |
| H4H12183P2 | 274 | 276 | 278 | 280 | 282 | 284 | 286 | 288 |
| H2M11682N | 290 | 292 | 294 | 296 | 298 | 300 | 302 | 304 |
| H2M11684N | 306 | 308 | 310 | 312 | 314 | 316 | 318 | 320 |
| H2M11694N | 322 | 324 | 326 | 328 | 330 | 332 | 334 | 336 |
| H2M11695N | 338 | 340 | 342 | 344 | 346 | 348 | 350 | 352 |

*Antibodies and fragments may include one or more variants of the sequences. See WO2017/218515

Polynucleotides encoding the chains set forth in Table A are set forth below in Table B.

TABLE B
Anti-C5 Antibody Chain Nucleotide Sequences*

| Antibody | SEQ ID NOS | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 1 | 3 | 5 | 7 | 9 | 11 | 13 | 15 |
| H2M11686N | 17 | 19 | 21 | 23 | 25 | 27 | 29 | 31 |
| H4H12159P | 33 | 35 | 37 | 39 | 41 | 43 | 45 | 47 |
| H4H12161P | 49 | 51 | 53 | 55 | 57 | 59 | 61 | 63 |
| H4H12163P | 65 | 67 | 69 | 71 | 73 | 75 | 77 | 79 |
| H4H12164P | 81 | 83 | 85 | 87 | 89 | 91 | 93 | 95 |
| H4H12166P | 97 | 99 | 101 | 103 | 105 | 107 | 109 | 111 |
| H4H12166P2 | 97 | 99 | 101 | 103 | 113 | 115 | 117 | 119 |
| H4H12166P3 | 121 | 123 | 125 | 127 | 105 | 107 | 109 | 111 |
| H4H12166P4 | 97 | 99 | 101 | 103 | 129 | 131 | 133 | 135 |
| H4H12166P5 | 137 | 139 | 141 | 143 | 105 | 107 | 109 | 111 |
| H4H12166P6 | 145 | 147 | 149 | 151 | 105 | 107 | 109 | 111 |
| H4H12166P7 | 121 | 123 | 125 | 127 | 129 | 131 | 133 | 135 |
| H4H12166P8 | 145 | 147 | 149 | 151 | 113 | 115 | 117 | 119 |
| H4H12166P9 | 145 | 147 | 149 | 151 | 129 | 131 | 133 | 135 |
| H4H12166P10 | 137 | 139 | 141 | 143 | 129 | 131 | 133 | 135 |
| H4H12167P | 153 | 155 | 157 | 159 | 161 | 163 | 165 | 167 |
| H4H12168P | 169 | 171 | 173 | 175 | 177 | 179 | 181 | 183 |
| H4H12169P | 185 | 187 | 189 | 191 | 193 | 195 | 197 | 199 |
| H4H12170P | 201 | 203 | 205 | 207 | 209 | 211 | 213 | 215 |
| H4H12171P | 217 | 219 | 221 | 223 | 225 | 227 | 229 | 231 |
| H4H12175P | 233 | 235 | 237 | 239 | 241 | 243 | 245 | 247 |
| H4H12176P2 | 249 | 251 | 253 | 255 | 257 | 259 | 261 | 263 |
| H4H12177P2 | 265 | 267 | 269 | 271 | 257 | 259 | 261 | 263 |
| H4H12183P2 | 273 | 275 | 277 | 279 | 281 | 283 | 285 | 287 |
| H2M11682N | 289 | 291 | 293 | 295 | 297 | 299 | 301 | 303 |
| H2M11684N | 305 | 307 | 309 | 311 | 313 | 315 | 317 | 319 |
| H2M11694N | 321 | 323 | 325 | 327 | 329 | 331 | 333 | 335 |
| H2M11695N | 337 | 339 | 341 | 343 | 345 | 347 | 349 | 351 |

*Antibodies and fragments may include one or more variants of
the sequences.
See WO2017/218515
H2M11683N
HCVR
QVQLVESGGGVVQPGRSLRLSCVASGFTFSSYGIHWVRQAPGKGLEWVAVIWDDGNNINY
SDSVKGRFIISRDNSRKTVYLQMNSLRGEDTAVYYCARDAPIAPVPDYWGQGTLVTVSS
(SEQ ID NO: 2)
HCDR1, HCDR2 and HCDR3 are set forth below, respectively:
Gly Phe Thr Phe Ser Ser Tyr Gly;
Ile Trp Asp Asp Gly Asn Asn Ile; and
Ala Arg Asp Ala Pro Ile Ala Pro Val Pro Asp Tyr
LCVR
DIQMTQSPSTLSASVGDRVTITCRASQSISSWLAWYQQKPGKAPKLLIYKASSLDTGVPS
RFSGSGSGTEFTLTISSLQPDDFATYYCQQYNTYSYTFGLGTKLEIK
(SEQ ID NO: 10)
LCDR1, LCDR2 and LCDR3 are set forth below, respectively:
Gln Ser Ile Ser Ser Trp;
Lys Ala Ser; and
Gln Gln Tyr Asn Thr Tyr Ser Tyr Thr
H2M11686N
HCVR
QVQLVESGGGLVKPGGSLRLSCAASGFTFSDYYMSWIRQAPGKGLEWVSYISSSGNTIKY
ADSMKGRFTISRDNAKKSLFVEMNSLRAEDTAVYYCARYKSSSDYFDHWGQGTLVTVSS
(SEQ ID NO: 18)
HCDR1, HCDR2 and HCDR3 are set forth below, respectively:
Gly Phe Thr Phe Ser Asp Tyr Tyr;
Ile Ser Ser Ser Gly Asn Thr Ile; and
Ala Arg Tyr Lys Ser Ser Ser Asp Tyr Phe Asp His
LCVR
EIVLTQSPATLSLSPGERATLSCRASQSVRSYLAWYQQKPGQAPRLLIYDASNRATAIPA
RFSGSGSGTDFTLTISSLEPEDLAVYYCQQSGNWPLTFGGGTKVEIK
(SEQ ID NO: 26)
LCDR1, LCDR2 and LCDR3 are set forth below, respectively:
Gln Ser Val Arg Ser Tyr;
Asp Ala Ser; and
Gln Gln Ser Gly Asn Trp Pro Leu Thr
H4H12159P
HCVR
QVQLVESGGGVVQPGRSLRLSCGASGFTFSTYGMHWVRQAPGKGLEWVAVIWDDGNNKYY
ADSVKGRFTISRDNSKNTLYLQMNSLRAEDTAVYYCARDSEVAPVGDYWGQGTLVTVSS
(SEQ ID NO: 34)
HCDR1, HCDR2 and HCDR3 are set forth below, respectively:
Gly Phe Thr Phe Ser Thr Tyr Gly;
Ile Trp Asp Asp Gly Asn Asn Lys; and
Ala Arg Asp Ser Glu Val Ala Pro Val Gly Asp Tyr
LCVR
DIQMTQSPSTLSASVGDRVTIICRASQSINRWLAWYQQKPGKAPKLLIYKASSLESGVPS
RFSGSGSGTEFTLTISSLQPDDFAAYYCQQYNDYSYTFGQGTKLEIK
(SEQ ID NO: 42)
LCDR1, LCDR2 and LCDR3 are set forth below, respectively:
Gln Ser Ile Asn Arg Trp;
Lys Ala Ser; and
Gln Gln Tyr Asn Asp Tyr Ser Tyr Thr
H4H12161P
HCVR
EVQLVESGGDLVQPGGSLRLSCAASGFTFSDHYMDWVRQAPGKGLDWIGRIRNKANAYNT
EYAASVRGRFTISRDDSQNLLYLQMNSLKTDDTAVYYCVRVWNYAYFAMDVWGQGTTVTV
SS
(SEQ ID NO: 50)
HCDR1, HCDR2 and HCDR3 are set forth below, respectively:
Gly Phe Thr Phe Ser Asp His Tyr;
Ile Arg Asn Lys Ala Asn Ala Tyr Asn Thr; and
Val Arg Val Trp Asn Tyr Ala Tyr Phe Ala Met Asp Val
LCVR
DIQMTQSPSSLSASVGDRVTITCRSSQNIGIFLNWYQQKPGEAPNLLISAASSLHSGVPS
RFSGSGSGTDFTLTIGSLQPEDFATYYCQQTYNTIFTFGPGTKVDIK
(SEQ ID NO: 58)
LCDR1, LCDR2 and LCDR3 are set forth below, respectively:
Gln Asn Ile Gly Ile Phe;
Ala Ala Ser; and
Gln Gln Thr Tyr Asn Thr Ile Phe Thr
H4H12163P
HCVR
EVQLVESGGDLVQPGGSLRLSCAASGFTFSSYAMNWVRQGPGKGLEWVSAISGRGDSTYY
ADSVKGRLTISRDNSKNTLYLQMNSLRAEDTAVYYCVKEGEQLVYWYFDLWGRGTLVTVS
S
(SEQ ID NO: 66)
HCDR1, HCDR2 and HCDR3 are set forth below, respectively:
Gly Phe Thr Phe Ser Ser Tyr Ala;
Ile Ser Gly Arg Gly Asp Ser Thr; and
Val Lys Glu Gly Glu Gln Leu Val Tyr Trp Tyr Phe Asp Leu
LCVR
DIQMTQSPSSLSASVGDRVTITCRASQTISNFLHWYQQKPGKAPKLLIYAASSLQSGVPS
RFSGSGSGTDFTLTISSLQPEDFSTYFCQQSYTTPLTFGGGTKVEIK
(SEQ ID NO: 74)
LCDR1, LCDR2 and LCDR3 are set forth below, respectively:
Gln Thr Ile Ser Asn Phe;
Ala Ala Ser; and
Gln Gln Ser Tyr Thr Thr Pro Leu Thr
H4H12164P
HCVR
EVQLVESGGGLVRSGGSLRLSCAASGFTFNRYAMTWVRQAPGKGLEWVSAISGSGSSTYY
TDSVKGRFTISRDNSKNSVDLQMHSLRVEDTAIYYCARGTTVTTGYGMDVWGQGTTVTVS
S
(SEQ ID NO: 82)
HCDR1, HCDR2 and HCDR3 are set forth below, respectively:
Gly Phe Thr Phe Asn Arg Tyr Ala;
Ile Ser Gly Ser Gly Ser Ser Thr; and
Ala Arg Gly Thr Thr Val Thr Thr Gly Tyr Gly Met Asp Val
LCVR
DIQMTQSPSSLSASVGDRVTFTCQASQDITNSLNWYQQKPGRAPKLLIYDASYLKAGVPS
RFSGSGSGTDFTFTISSLQPEDIATYYCQQYDDLPYTFGQGTKLEIK
(SEQ ID NO: 90)
LCDR1, LCDR2 and LCDR3 are set forth below, respectively:
Gln Asp Ile Thr Asn Ser;
Asp Ala Ser; and
Gln Gln Tyr Asp Asp Leu Pro Tyr Thr
H4H12166P
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIFDYWGQGTLVTVSS
(SEQ ID NO: 98)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp Thr Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
(SEQ ID NO: 106)
LCDR1, LCDR2 and LCDR3 are set forth below, respectively:
Gln Gly Ile Arg Asn Asp;
Ala Ala Ser; and
Leu Gln Asp Phe Asn Tyr Pro Trp Thr
H4H12166P2
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIFDYWGQGTLVTVSS
(SEQ ID NO: 98)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp Thr Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCHQDFNYPWTFGQGTKVEIK
(SEQ ID NO: 114)
LCDR1, LCDR2 and LCDR3 are set forth below, respectively:
Gln Gly Ile Arg Asn Asp;
Ala Ala Ser; and
His Gln Asp Phe Asn Tyr Pro Trp Thr
H4H12166P3
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREHNVDTTMIFDYWGQGTLVTVSS
(SEQ ID NO: 122)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu His Asn Val Asp Thr Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
(SEQ ID NO: 106)
LCDR1, LCDR2 and LCDR3 are set forth below, respectively:
Gln Gly Ile Arg Asn Asp;
Ala Ala Ser; and
Leu Gln Asp Phe Asn Tyr Pro Trp Thr
H4H12166P4
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIFDYWGQGTLVTVSS
(SEQ ID NO: 98)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp Thr Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
(SEQ ID NO: 130)
LCDR1, LCDR2 and LCDR3 are set forth below, respectively:
Gln Gly Ile Arg Asn Asp;
Ala Ala Ser; and
Leu Gln Asp Phe Asn Tyr Pro Trp His
H4H12166P5
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIHDYWGQGTLVTVSS
(SEQ ID NO: 138)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp Thr Thr Met Ile His Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
(SEQ ID NO: 106)
LCDR1, LCDR2 and LCDR3 are set forth below, respectively:
Gln Gly Ile Arg Asn Asp;
Ala Ala Ser; and
Leu Gln Asp Phe Asn Tyr Pro Trp Thr
H4H12166P6
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDHTMIFDYWGQGTLVTVSS
(SEQ ID NO: 146)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp His Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
(SEQ ID NO: 106)
LCDR1, LCDR2 and LCDR3 are set forth below, respectively:
Gln Gly Ile Arg Asn Asp;
Ala Ala Ser; and
Leu Gln Asp Phe Asn Tyr Pro Trp Thr
H4H12166P7
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREHNVDTTMIFDYWGQGTLVTVSS
(SEQ ID NO: 122)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu His Asn Val Asp Thr Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
(SEQ ID NO: 130)
LCDR1, LCDR2 and LCDR3 are set forth below, respectively:
Gln Gly Ile Arg Asn Asp;
Ala Ala Ser; and
Leu Gln Asp Phe Asn Tyr Pro Trp His
H4H12166P8
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDHTMIFDYWGQGTLVTVSS
(SEQ ID NO: 146)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp His Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCHQDFNYPWTFGQGTKVEIK
(SEQ ID NO: 114)
LCDR1, LCDR2 and LCDR3 are set forth below, respectively:
Gln Gly Ile Arg Asn Asp;
Ala Ala Ser; and
His Gln Asp Phe Asn Tyr Pro Trp Thr
H4H12166P9
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDHTMIFDYWGQGTLVTVSS
(SEQ ID NO: 146)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp His Thr Met Ile Phe Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
(SEQ ID NO: 130)
LCDR1, LCDR2 and LCDR3 are set forth below, respectively:
Gln Gly Ile Arg Asn Asp;
Ala Ala Ser; and
Leu Gln Asp Phe Asn Tyr Pro Trp His
H4H12166P10
HCVR
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIHDYWGQGTLVTVSS
(SEQ ID NO: 138)
HCDR1, HCDR2 and HCDR3 are set forth below, respectively:
Gly Asp Ser Val Ser Ser Ser Tyr;
Ile Tyr Tyr Ser Gly Ser Ser; and
Ala Arg Glu Gly Asn Val Asp Thr Thr Met Ile His Asp Tyr
LCVR
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
(SEQ ID NO: 130)
LCDR1, LCDR2 and LCDR3 are set forth below, respectively:
Gln Gly Ile Arg Asn Asp;
Ala Ala Ser; and
Leu Gln Asp Phe Asn Tyr Pro Trp His
H4H12167P
HCVR
QVQLVESGGGLVKPGGSLRLSCAASGFTFSDSYMSWIRQAPGKGLEWISYIGSSGNTFYY
ADSVKGRFTISRDNANNLLYLQMTSLRAEDTAVYYCAREEGDFWSAVDSWGQGTLVTVSS
(SEQ ID NO: 154)
HCDR1, HCDR2 and HCDR3 are set forth below, respectively:
Gly Phe Thr Phe Ser Asp Ser Tyr;
Ile Gly Ser Ser Gly Asn Thr Phe; and
Ala Arg Glu Glu Gly Asp Phe Trp Ser Ala Val Asp Ser
LCVR
DIQLTQSPSFLSASVGDRVTITCWASQGISSYLAWYQQKPGKAPKLLIHTASTLQSGVPS
RFSGSGSGTEFTLTISNLQPEDFATYYCQQLNSYPFTFGPGTKVDIK
(SEQ ID NO: 162)
LCDR1, LCDR2 and LCDR3 are set forth below, respectively:
Gln Gly Ile Ser Ser Tyr;
Thr Ala Ser; and
Gln Gln Leu Asn Ser Tyr Pro Phe Thr
H4H12168P
HCVR
QVQLVESGGGVVQPGGSLRLSCAASGFTFGGHAMHWVRQAPGKGLEWLAVISSDGSNKQY
ADSVKGRFTISRDNPKNTLYLQMNSLRVGDTAIYYCAKEVAPRYYYYGLDVWGQGTTVTV
SS
(SEQ ID NO: 170)
HCDR1, HCDR2 and HCDR3 are set forth below, respectively:
Gly Phe Thr Phe Gly Gly His Ala;
Ile Ser Ser Asp Gly Ser Asn Lys; and
Ala Lys Glu Val Ala Pro Arg Tyr Tyr Tyr Tyr Gly Leu Asp Val
LCVR
DIQMTQSPSSLSASVGDRVTITCRASQDISNFLAWYQQKPGKVPKLLIYTASTLQSGVPS
RFSGSGSGTDFTLTVSSLQPEDVATYYCQKYAGALTFGPGTKVDIK
(SEQ ID NO: 178)
LCDR1, LCDR2 and LCDR3 are set forth below, respectively:
Gln Asp Ile Ser Asn Phe;
Thr Ala Ser; and
Gln Lys Tyr Ala Gly Ala Leu Thr
H4H12169P
HCVR
EVQLVESGGGLAQPGGSLRLSCAASGFTFRSYAMSWVRQAPGKGPEWVSGIGGNGVTTYY
ADSVKGRFTISRDNSKNTLFLQMNSLRAEDTAVYYCVQGGLGGYFTGYWGQGTLVTVSS
(SEQ ID NO: 186)
HCDR1, HCDR2 and HCDR3 are set forth below, respectively:
Gly Phe Thr Phe Arg Ser Tyr Ala;
Ile Gly Gly Asn Gly Val Thr Thr; and
Val Gln Gly Gly Leu Gly Gly Tyr Phe Thr Gly Tyr
LCVR
DIQMTQSPSSLSASVGDRVTITCRASQSISTYLNWYQQNPGKAPKLLIFDASSLQSGVPS
RFSGSGSGTDFTLTIRGLQPEDFATYYCQQSYSAPLTFGGGTKVEIK
(SEQ ID NO: 194)
LCDR1, LCDR2 and LCDR3 are set forth below, respectively:
Gln Ser Ile Ser Thr Tyr;
Asp Ala Ser; and
Gln Gln Ser Tyr Ser Ala Pro Leu Thr
H4H12170P
HCVR
QVQLVESGGGVVQPGRSLRLSCAASGFTFSGYGMHWVRQAPGKGLEWVALIWLDGSNDYY
ADSVKGRFTISRDNSKNTLYLQMNRLRAEDTAVYYCARDGPVAAIPDYWGQGTLVTVSS
(SEQ ID NO: 202)
HCDR1, HCDR2 and HCDR3 are set forth below, respectively:
Gly Phe Thr Phe Ser Gly Tyr Gly;
Ile Trp Leu Asp Gly Ser Asn Asp; and
Ala Arg Asp Gly Pro Val Ala Ala Ile Pro Asp Tyr
LCVR
DIQMTQSPSTLSASVGDRVTITCRASQSISRWLAWYQLKPGKAPKLLIYKASSLESGVPS
RFSGSGSGTDFTLTISSLQPDDFATYYCQQYNTYSYTFGQGTKLEIK
(SEQ ID NO: 210)
LCDR1, LCDR2 and LCDR3 are set forth below, respectively:
Gln Ser Ile Ser Arg Trp;
Lys Ala Ser; and
Gln Gln Tyr Asn Thr Tyr Ser Tyr Thr
H4H12171P
HCVR
EVQLVESGGGVVRPGGSLRLSCAASGFTFDEYGMTWVRQVPGKGLEWVSGITWNGGFTDY
TDSVKGRFTSSRDNAKNSLYLQMNSLRAEDTALYYCARDGYSSSWGAYDIWGQGTMVTVSS
(SEQ ID NO: 218)
HCDR1, HCDR2 and HCDR3 are set forth below, respectively:
Gly Phe Thr Phe Asp Glu Tyr Gly;
Ile Thr Trp Asn Gly Gly Phe Thr; and
Ala Arg Asp Gly Tyr Ser Ser Ser Trp Gly Ala Tyr Asp Ile
LCVR
DIQMTQSPSSLSASVGDRVTITCRASQSISTYLNWYQQKPGKAPKLLIYAASSLQSGVPL
RFSGSGSGTDFTLTISSLQPEDFASYFCQQSYSTPYTFGQGTKLEIK
(SEQ ID NO: 226)
LCDR1, LCDR2 and LCDR3 are set forth below, respectively:
Gln Ser Ile Ser Thr Tyr;
Ala Ala Ser; and
Gln Gln Ser Tyr Ser Thr Pro Tyr Thr
H4H12175P
HCVR
EVQLVESGGGVVQPGGSLRLSCAASGFTFNDYAMHWVRQAPGKGLEWVSLISGDGGNTYY
ADSVKGRLTISRDNSKNSLYLQMNSLRTEDTALYYCAKDKGWNFGYFDLWGRGTLVTVSS
(SEQ ID NO: 234)
HCDR1, HCDR2 and HCDR3 are set forth below, respectively:
Gly Phe Thr Phe Asn Asp Tyr Ala;
Ile Ser Gly Asp Gly Gly Asn Thr; and
Ala Lys Asp Lys Gly Trp Asn Phe Gly Tyr Phe Asp Leu
LCVR
DIQMTQSPSSLSTSVGDRVTITCRASQNIDTYLNWYQQKPGKAPKLLIYDASSLQSGVPS
RFSGSGSGTDFTLTITSLQPEDFATYYCQQNDNILHPLTFGGGTKVEIK
(SEQ ID NO: 242)
LCDR1, LCDR2 and LCDR3 are set forth below, respectively:
Gln Asn Ile Asp Thr Tyr;
Asp Ala Ser; and
Gln Gln Asn Asp Asn Ile Leu His Pro Leu Thr
H4H12176P2
HCVR
EVQLVESGGGLVQPGGSLRLSCAASGFHSNRYWMDWVRQAPGKGLEWVANIKQDGSEENY
VDSVKGRFTISRDNAKNSLYLQMNSLRAEDTAVYYCARDRSTSWVPYWFFDLWGRGTLVTVSS
(SEQ ID NO: 250)
HCDR1, HCDR2 and HCDR3 are set forth below, respectively:
Gly Phe His Ser Asn Arg Tyr Trp;
Ile Lys Gln Asp Gly Ser Glu Glu; and
Ala Arg Asp Arg Ser Thr Ser Trp Val Pro Tyr Trp Phe Phe Asp Leu
LCVR
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
(SEQ ID NO: 258)
LCDR1, LCDR2 and LCDR3 are set forth below, respectively:
Gln Ser Ile Ser Ser Tyr;
Ala Ala Ser; and
Gln Gln Ser Tyr Ser Thr Pro Pro Ile Thr
H4H12177P2
HCVR
EVQLVESGGGVVQRGESLRLSCSASDFIFKDYAMYWVRQIPGKGLEWISLISGDGDTTWY
GDSVKGRFTISRDNNENSLFLQMNDLRTEDTAMYYCARDMGWNFFQLQYWGQGTLVTVSS
(SEQ ID NO: 266)
HCDR1, HCDR2 and HCDR3 are set forth below, respectively:
Asp Phe Ile Phe Lys Asp Tyr Ala;
Ile Ser Gly Asp Gly Asp Thr Thr; and
Ala Arg Asp Met Gly Trp Asn Phe Phe Gln Leu Gln Tyr
LCVR
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
(SEQ ID NO: 258)
LCDR1, LCDR2 and LCDR3 are set forth below, respectively:
Gln Ser Ile Ser Ser Tyr;
Ala Ala Ser; and
Gln Gln Ser Tyr Ser Thr Pro Pro Ile Thr
H4H12183P2
HCVR
QVQLQESGPALVKPSQTLSLTCTVSGGSIIRGSTYWSWVRQFPGKGLEWIGYSYYSGTAY
YNPSLESRATISVDTSKNQFSLNLKSVTAADTAVYYCTREIGVAGLFDIWGQGTLVTVSS
(SEQ ID NO: 274)
HCDR1, HCDR2 and HCDR3 are set forth below, respectively:
Gly Gly Ser Ile Ile Arg Gly Ser Thr Tyr;
Ser Tyr Tyr Ser Gly Thr Ala; and
Thr Arg Glu Ile Gly Val Ala Gly Leu Phe Asp Ile
LCVR
EIVLTQSPGTLSLSPGERATLSCRASQSVSSSYLAWYQQKPGQAPRLLIYGASSRATGIP
DRFSGSGSGTDFTLTISRLEPEDFAVYYCQQYGSSPWTFGQGTKVEIK
(SEQ ID NO: 282)
LCDR1, LCDR2 and LCDR3 are set forth below, respectively:
Arg Ala Ser Gln Ser Val Ser Ser Ser Tyr Leu Ala;
Gly Ala Ser Ser Arg Ala Thr; and
Gln Gln Tyr Gly Ser Ser Pro Trp Thr
H2M11682N
HCVR
QEQLVQSGAEVKKPGASVKVSCKASGYTFTGYYIHWVRQAPGLGLEWMGWINPNSGGTKY
AQKFQGRVTMTRDTSINTAYMELKRLKSDDSAVYYCARDAPPHDVFDIWGQGTLVTVSS
(SEQ ID NO: 290)
HCDR1, HCDR2 and HCDR3 are set forth below, respectively:
Gly Tyr Thr Phe Thr Gly Tyr Tyr;
Ile Asn Pro Asn Ser Gly Gly Thr; and
Ala Arg Asp Ala Pro Pro His Asp Val Phe Asp Ile
LCVR
DIQMTQSPSSLSASVGDRVTITCRASQGIRNDLGWYQQKPGKAPKRLIYAASSLQIGVPS
RFSGSGSGTEFTLTISSLQPEDFATYYCLQHNSYPLTFGGGTKVEIK
(SEQ ID NO: 298)
LCDR1, LCDR2 and LCDR3 are set forth below, respectively:
Gln Gly Ile Arg Asn Asp;
Ala Ala Ser; and
Leu Gln His Asn Ser Tyr Pro Leu Thr
H2M11684N
HCVR
QVQLQESGPGLVKPSQTLSLTCTVSGGSISSGAYHWSWIRQHPGKGLEWIGYIYYNGDTY
YNPSLKSRVTISVDTSKNQFFLKVTSVTAADTAMYYCAGEKQLTAFDIWGQGTLVTVSS
(SEQ ID NO: 306)
HCDR1, HCDR2 and HCDR3 are set forth below, respectively:
Gly Gly Ser Ile Ser Ser Gly Ala Tyr His;
Ile Tyr Tyr Asn Gly Asp Thr; and
Ala Gly Glu Lys Gln Leu Thr Ala Phe Asp Ile
LCVR
VIQMTQSPSSLSASVGDRVTITCRASQDINNFLNWYQQKLGKAPKLLISDASNLQTGVPS
RFSGSGSGTDFTFTISSLQPEDIAAYYCQQYDHFPYTFGQGTRLENN
(SEQ ID NO: 314)
LCDR1, LCDR2 and LCDR3 are set forth below, respectively:
Gln Asp Ile Asn Asn Phe;
Asp Ala Ser; and
Gln Gln Tyr Asp His Phe Pro Tyr Thr
H2M11694N
HCVR
EVQLVESGGGVVRPGGSLRLSCAASGFTFDDYGMTWVRQAPGKGLEWVSGINWNGDSTEY
SDSVKGRFTISRDNAKNSLYLQMNSLRAEDTAFYHCARENNWNFYFDYWGQGTLVTVSS
(SEQ ID NO: 322)
HCDR1, HCDR2 and HCDR3 are set forth below, respectively:
Gly Phe Thr Phe Asp Asp Tyr Gly;
Ile Asn Trp Asn Gly Asp Ser Thr; and
Ala Arg Glu Asn Asn Trp Asn Phe Tyr Phe Asp Tyr
LCVR
EIVMTQSPATLSVSRGERATLSCRASQSVSSNLAWYQQKLGQAPRLLIYGASTRATGIPA
RFSGSGSGTEFTLTISSLQSEDFAVYYCQQYNNWPWTFGQGTKVEIK
(SEQ ID NO: 330)
LCDR1, LCDR2 and LCDR3 are set forth below, respectively:
Gln Ser Val Ser Ser Asn;
Gly Ala Ser; and
Gln Gln Tyr Asn Asn Trp Pro Trp Thr
H2M11695N
HCVR
QVHLVQSGAEVKKPGASVKVSCKVSGNTLTELSMHWVRQAPGKGLEWMGGFDPEDGDTIY
SQKFQGRVTLTEDTSTDTAYMELSSLRSEDTAVYYCSTVGGPTSDCWGQGTLVTVSS
(SEQ ID NO: 338)
HCDR1, HCDR2 and HCDR3 are set forth below, respectively:
Gly Asn Thr Leu Thr Glu Leu Ser;
Phe Asp Pro Glu Asp Gly Asp Thr; and
Ser Thr Val Gly Gly Pro Thr Ser Asp Cys
LCVR
DIQMTQSPSSLSASVGDRVTITCQASQDISNYLNWYQQKPGKAPKVLIFDASNLEPGVPS
RFSGSGSGTDFTFTIISLQPEDIATYYCQQYDNLPITFGQGTRLDIK
(SEQ ID NO: 346)
LCDR1, LCDR2 and LCDR3 are set forth below, respectively:
Gln Asp Ile Ser Asn Tyr;
Asp Ala Ser; and
Gln Gln Tyr Asp Asn Leu Pro Ile Thr

### CDRs Underscored

In an embodiment of the disclosure, an antibody or antigen-binding fragment thereof that binds specifically to C5, which is in a co-formulation of the present disclosure comprises:

(1)
a HCVR that comprises the HCDR1, HCDR2 and HCDR3 of a HCVR that comprises the amino acid sequence set forth in SEQ ID NO: 2 (or a variant thereof), and
a LCVR that comprises the LCDR1, LCDR2 and LCDR3 of a LCVR that comprises the amino acid sequence set forth in SEQ ID NO: 10 (or a variant thereof);
   (2)
a HCVR that comprises the HCDR1, HCDR2 and HCDR3 of a HCVR that comprises the amino acid sequence set forth in SEQ ID NO: 18 (or a variant thereof), and
a LCVR that comprises the LCDR1, LCDR2 and LCDR3 of a LCVR that comprises the amino acid sequence set forth in SEQ ID NO: 26 (or a variant thereof);
   (3)
a HCVR that comprises the HCDR1, HCDR2 and HCDR3 of a HCVR that comprises the amino acid sequence set forth in SEQ ID NO: 34 (or a variant thereof), and
a LCVR that comprises the LCDR1, LCDR2 and LCDR3 of a LCVR that comprises the amino acid sequence set forth in SEQ ID NO: 42 (or a variant thereof);
   (4)
a HCVR that comprises the HCDR1, HCDR2 and HCDR3 of a HCVR that comprises the amino acid sequence set forth in SEQ ID NO: 50 (or a variant thereof), and
a LCVR that comprises the LCDR1, LCDR2 and LCDR3 of a LCVR that comprises the amino acid sequence set forth in SEQ ID NO: 58 (or a variant thereof);
   (5)
a HCVR that comprises the HCDR1, HCDR2 and HCDR3 of a HCVR that comprises the amino acid sequence set forth in SEQ ID NO: 66 (or a variant thereof), and
a LCVR that comprises the LCDR1, LCDR2 and LCDR3 of a LCVR that comprises the amino acid sequence set forth in SEQ ID NO: 74 (or a variant thereof);
   (6)
a HCVR that comprises the HCDR1, HCDR2 and HCDR3 of a HCVR that comprises the amino acid sequence set forth in SEQ ID NO: 82 (or a variant thereof), and
a LCVR that comprises the LCDR1, LCDR2 and LCDR3 of a LCVR that comprises the amino acid sequence set forth in SEQ ID NO: 90 (or a variant thereof);
   (7)
a HCVR that comprises the HCDR1, HCDR2 and HCDR3 of a HCVR that comprises the amino acid sequence set forth in SEQ ID NO: 98 (or a variant thereof), and
a LCVR that comprises the LCDR1, LCDR2 and LCDR3 of a LCVR that comprises the amino acid sequence set forth in SEQ ID NO: 106 (or a variant thereof);
   (8)
a HCVR that comprises the HCDR1, HCDR2 and HCDR3 of a HCVR that comprises the amino acid sequence set forth in SEQ ID NO: 98 (or a variant thereof), and
a LCVR that comprises the LCDR1, LCDR2 and LCDR3 of a LCVR that comprises the amino acid sequence set forth in SEQ ID NO: 114 (or a variant thereof);
   (9)
a HCVR that comprises the HCDR1, HCDR2 and HCDR3 of a HCVR that comprises the amino acid sequence set forth in SEQ ID NO: 122 (or a variant thereof), and
a LCVR that comprises the LCDR1, LCDR2 and LCDR3 of a LCVR that comprises the amino acid sequence set forth in SEQ ID NO: 106 (or a variant thereof);
   (10)
a HCVR that comprises the HCDR1, HCDR2 and HCDR3 of a HCVR that comprises the amino acid sequence set forth in SEQ ID NO: 98 (or a variant thereof), and
a LCVR that comprises the LCDR1, LCDR2 and LCDR3 of a LCVR that comprises the amino acid sequence set forth in SEQ ID NO: 130 (or a variant thereof);
   (11)
a HCVR that comprises the HCDR1, HCDR2 and HCDR3 of a HCVR that comprises the amino acid sequence set forth in SEQ ID NO: 138 (or a variant thereof), and
a LCVR that comprises the LCDR1, LCDR2 and LCDR3 of a LCVR that comprises the amino acid sequence set forth in SEQ ID NO: 106 (or a variant thereof);
   (12)
a HCVR that comprises the HCDR1, HCDR2 and HCDR3 of a HCVR that comprises the amino acid sequence set forth in SEQ ID NO: 146 (or a variant thereof), and
a LCVR that comprises the LCDR1, LCDR2 and LCDR3 of a LCVR that comprises the amino acid sequence set forth in SEQ ID NO: 106 (or a variant thereof);
   (13)
a HCVR that comprises the HCDR1, HCDR2 and HCDR3 of a HCVR that comprises the amino acid sequence set forth in SEQ ID NO: 122 (or a variant thereof), and
a LCVR that comprises the LCDR1, LCDR2 and LCDR3 of a LCVR that comprises the amino acid sequence set forth in SEQ ID NO: 130 (or a variant thereof);
   (14)
a HCVR that comprises the HCDR1, HCDR2 and HCDR3 of a HCVR that comprises the amino acid sequence set forth in SEQ ID NO: 146 (or a variant thereof), and
a LCVR that comprises the LCDR1, LCDR2 and LCDR3 of a LCVR that comprises the amino acid sequence set forth in SEQ ID NO: 114 (or a variant thereof);
   (15)
a HCVR that comprises the HCDR1, HCDR2 and HCDR3 of a HCVR that comprises the amino acid sequence set forth in SEQ ID NO: 146 (or a variant thereof), and
a LCVR that comprises the LCDR1, LCDR2 and LCDR3 of a LCVR that comprises the amino acid sequence set forth in SEQ ID NO: 130 (or a variant thereof);
   (16)
a HCVR that comprises the HCDR1, HCDR2 and HCDR3 of a HCVR that comprises the amino acid sequence set forth in SEQ ID NO: 138, and
a LCVR that comprises the LCDR1, LCDR2 and LCDR3 of a LCVR that comprises the amino acid sequence set forth in SEQ ID NO: 130 (or a variant thereof);
   (17)
a HCVR that comprises the HCDR1, HCDR2 and HCDR3 of a HCVR that comprises the amino acid sequence set forth in SEQ ID NO: 154 (or a variant thereof), and
a LCVR that comprises the LCDR1, LCDR2 and LCDR3 of a LCVR that comprises the amino acid sequence set forth in SEQ ID NO: 162 (or a variant thereof);
   (18)
a HCVR that comprises the HCDR1, HCDR2 and HCDR3 of a HCVR that comprises the amino acid sequence set forth in SEQ ID NO: 170 (or a variant thereof), and
a LCVR that comprises the LCDR1, LCDR2 and LCDR3 of a LCVR that comprises the amino acid sequence set forth in SEQ ID NO: 178 (or a variant thereof);
   (19)
a HCVR that comprises the HCDR1, HCDR2 and HCDR3 of a HCVR that comprises the amino acid sequence set forth in SEQ ID NO: 186 (or a variant thereof), and
a LCVR that comprises the LCDR1, LCDR2 and LCDR3 of a LCVR that comprises the amino acid sequence set forth in SEQ ID NO: 194 (or a variant thereof);
   (20)
a HCVR that comprises the HCDR1, HCDR2 and HCDR3 of a HCVR that comprises the amino acid sequence set forth in SEQ ID NO: 202 (or a variant thereof), and
a LCVR that comprises the LCDR1, LCDR2 and LCDR3 of a LCVR that comprises the amino acid sequence set forth in SEQ ID NO: 210 (or a variant thereof);
   (21)
a HCVR that comprises the HCDR1, HCDR2 and HCDR3 of a HCVR that comprises the amino acid sequence set forth in SEQ ID NO: 218 (or a variant thereof), and
a LCVR that comprises the LCDR1, LCDR2 and LCDR3 of a LCVR that comprises the amino acid sequence set forth in SEQ ID NO: 226 (or a variant thereof);
   (22)
a HCVR that comprises the HCDR1, HCDR2 and HCDR3 of a HCVR that comprises the amino acid sequence set forth in SEQ ID NO: 234 (or a variant thereof), and
a LCVR that comprises the LCDR1, LCDR2 and LCDR3 of a LCVR that comprises the amino acid sequence set forth in SEQ ID NO: 242 (or a variant thereof);
   (23)
a HCVR that comprises the HCDR1, HCDR2 and HCDR3 of a HCVR that comprises the amino acid sequence set forth in SEQ ID NO: 250 (or a variant thereof), and
a LCVR that comprises the LCDR1, LCDR2 and LCDR3 of a LCVR that comprises the amino acid sequence set forth in SEQ ID NO: 258 (or a variant thereof);
   (24)
a HCVR that comprises the HCDR1, HCDR2 and HCDR3 of a HCVR that comprises the amino acid sequence set forth in SEQ ID NO: 266 (or a variant thereof), and
a LCVR that comprises the LCDR1, LCDR2 and LCDR3 of a LCVR that comprises the amino acid sequence set forth in SEQ ID NO: 258 (or a variant thereof);
   (25)
a HCVR that comprises the HCDR1, HCDR2 and HCDR3 of a HCVR that comprises the amino acid sequence set forth in SEQ ID NO: 274 (or a variant thereof), and
a LCVR that comprises the LCDR1, LCDR2 and LCDR3 of a LCVR that comprises the amino acid sequence set forth in SEQ ID NO: 282 (or a variant thereof);
   (26)
a HCVR that comprises the HCDR1, HCDR2 and HCDR3 of a HCVR that comprises the amino acid sequence set forth in SEQ ID NO: 290 (or a variant thereof), and
a LCVR that comprises the LCDR1, LCDR2 and LCDR3 of a LCVR that comprises the amino acid sequence set forth in SEQ ID NO: 298 (or a variant thereof);
   (27)
a HCVR that comprises the HCDR1, HCDR2 and HCDR3 of a HCVR that comprises the amino acid sequence set forth in SEQ ID NO: 306 (or a variant thereof), and
a LCVR that comprises the LCDR1, LCDR2 and LCDR3 of a LCVR that comprises the amino acid sequence set forth in SEQ ID NO: 314 (or a variant thereof);
   (28)
a HCVR that comprises the HCDR1, HCDR2 and HCDR3 of a HCVR that comprises the amino acid sequence set forth in SEQ ID NO: 322 (or a variant thereof), and
a LCVR that comprises the LCDR1, LCDR2 and LCDR3 of a LCVR that comprises the amino acid sequence set forth in SEQ ID NO: 330 (or a variant thereof); and/or
   (29)
a HCVR that comprises the HCDR1, HCDR2 and HCDR3 of a HCVR that comprises the amino acid sequence set forth in SEQ ID NO: 338 (or a variant thereof), and
a LCVR that comprises the LCDR1, LCDR2 and LCDR3 of a LCVR that comprises the amino acid sequence set forth in SEQ ID NO: 346 (or a variant thereof).

In an embodiment of the disclosure, an antibody or antigen-binding fragment thereof that binds specifically to C5, which is in a co-formulation of the present disclosure comprises:

(a)
a heavy chain variable region comprising
an HCDR1 that comprises the amino acid sequence set forth in SEQ ID NO: 4 (or a variant thereof),
an HCDR2 that comprises the amino acid sequence set forth in SEQ ID NO: 6 (or a variant thereof),
an HCDR3 that comprises the amino acid sequence set forth in SEQ ID NO: 8 (or a variant thereof),
and a light chain variable region comprising
an LCDR1 that comprises the amino acid sequence set forth in SEQ ID NO: 12 (or a variant thereof),
an LCDR2 that comprises the amino acid sequence set forth in SEQ ID NO: 14 (or a variant thereof),
an LCDR3 that comprises the amino acid sequence set forth in SEQ ID NO: 16 (or a variant thereof);
   (b)
a heavy chain variable region comprising
an HCDR1 that comprises the amino acid sequence set forth in SEQ ID NO: 20 (or a variant thereof),
an HCDR2 that comprises the amino acid sequence set forth in SEQ ID NO: 22 (or a variant thereof),
an HCDR3 that comprises the amino acid sequence set forth in SEQ ID NO: 24 (or a variant thereof),
and a light chain variable region comprising
an LCDR1 that comprises the amino acid sequence set forth in SEQ ID NO: 28 (or a variant thereof),
an LCDR2 that comprises the amino acid sequence set forth in SEQ ID NO: 30 (or a variant thereof),
an LCDR3 that comprises the amino acid sequence set forth in SEQ ID NO: 32 (or a variant thereof);
(c)
a heavy chain variable region comprising
an HCDR1 that comprises the amino acid sequence set forth in SEQ ID NO: 36 (or a variant thereof),
an HCDR2 that comprises the amino acid sequence set forth in SEQ ID NO: 38 (or a variant thereof),
an HCDR3 that comprises the amino acid sequence set forth in SEQ ID NO: 40 (or a variant thereof),
and a light chain variable region comprising
an LCDR1 that comprises the amino acid sequence set forth in SEQ ID NO: 44 (or a variant thereof),
an LCDR2 that comprises the amino acid sequence set forth in SEQ ID NO: 46 (or a variant thereof),
an LCDR3 that comprises the amino acid sequence set forth in SEQ ID NO: 48 (or a variant thereof);
   (d)
a heavy chain variable region comprising
an HCDR1 that comprises the amino acid sequence set forth in SEQ ID NO: 52 (or a variant thereof),
an HCDR2 that comprises the amino acid sequence set forth in SEQ ID NO: 54 (or a variant thereof),
an HCDR3 that comprises the amino acid sequence set forth in SEQ ID NO: 56 (or a variant thereof),
and a light chain variable region comprising
an LCDR1 that comprises the amino acid sequence set forth in SEQ ID NO: 60 (or a variant thereof),
an LCDR2 that comprises the amino acid sequence set forth in SEQ ID NO: 62 (or a variant thereof),
an LCDR3 that comprises the amino acid sequence set forth in SEQ ID NO: 64 (or a variant thereof);
   (e)
a heavy chain variable region comprising
an HCDR1 that comprises the amino acid sequence set forth in SEQ ID NO: 68 (or a variant thereof),
an HCDR2 that comprises the amino acid sequence set forth in SEQ ID NO: 70 (or a variant thereof),
an HCDR3 that comprises the amino acid sequence set forth in SEQ ID NO: 72 (or a variant thereof),
and a light chain variable region comprising
an LCDR1 that comprises the amino acid sequence set forth in SEQ ID NO: 76 (or a variant thereof),
an LCDR2 that comprises the amino acid sequence set forth in SEQ ID NO: 78 (or a variant thereof),
an LCDR3 that comprises the amino acid sequence set forth in SEQ ID NO: 80 (or a variant thereof);
   (f)
a heavy chain variable region comprising
an HCDR1 that comprises the amino acid sequence set forth in SEQ ID NO: 84 (or a variant thereof),
an HCDR2 that comprises the amino acid sequence set forth in SEQ ID NO: 86 (or a variant thereof),
an HCDR3 that comprises the amino acid sequence set forth in SEQ ID NO: 88 (or a variant thereof),
and a light chain variable region comprising
an LCDR1 that comprises the amino acid sequence set forth in SEQ ID NO: 92 (or a variant thereof),
an LCDR2 that comprises the amino acid sequence set forth in SEQ ID NO: 94 (or a variant thereof),
an LCDR3 that comprises the amino acid sequence set forth in SEQ ID NO: 96 (or a variant thereof);
   (h)
a heavy chain variable region comprising
an HCDR1 that comprises the amino acid sequence set forth in SEQ ID NO: 100 (or a variant thereof),
an HCDR2 that comprises the amino acid sequence set forth in SEQ ID NO: 102 (or a variant thereof),
an HCDR3 that comprises the amino acid sequence set forth in SEQ ID NO: 104 (or a variant thereof),
and a light chain variable region comprising
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
   (j)
a heavy chain variable region comprising
an HCDR1 that comprises the amino acid sequence set forth in SEQ ID NO: 100 (or a variant thereof),
an HCDR2 that comprises the amino acid sequence set forth in SEQ ID NO: 102 (or a variant thereof),
an HCDR3 that comprises the amino acid sequence set forth in SEQ ID NO: 104 (or a variant thereof),
and a light chain variable region comprising
an LCDR1 that comprises the amino acid sequence set forth in SEQ ID NO: 116 (or a variant thereof),
an LCDR2 that comprises the amino acid sequence set forth in SEQ ID NO: 118 (or a variant thereof),
an LCDR3 that comprises the amino acid sequence set forth in SEQ ID NO: 120 (or a variant thereof);
   (k)
a heavy chain variable region comprising
an HCDR1 that comprises the amino acid sequence set forth in SEQ ID NO: 124 (or a variant thereof),
an HCDR2 that comprises the amino acid sequence set forth in SEQ ID NO: 126 (or a variant thereof),
an HCDR3 that comprises the amino acid sequence set forth in SEQ ID NO: 128 (or a variant thereof),
and a light chain variable region comprising
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
   (m)
a heavy chain variable region comprising
an HCDR1 that comprises the amino acid sequence set forth in SEQ ID NO: 100 (or a variant thereof),
an HCDR2 that comprises the amino acid sequence set forth in SEQ ID NO: 102 (or a variant thereof),
an HCDR3 that comprises the amino acid sequence set forth in SEQ ID NO: 104 (or a variant thereof),
and a light chain variable region comprising
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
   (n)
a heavy chain variable region comprising
an HCDR1 that comprises the amino acid sequence set forth in SEQ ID NO: 140 (or a variant thereof),
an HCDR2 that comprises the amino acid sequence set forth in SEQ ID NO: 142 (or a variant thereof),
an HCDR3 that comprises the amino acid sequence set forth in SEQ ID NO: 144 (or a variant thereof),
and a light chain variable region comprising
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
   (p)
a heavy chain variable region comprising
an HCDR1 that comprises the amino acid sequence set forth in SEQ ID NO: 148 (or a variant thereof),
an HCDR2 that comprises the amino acid sequence set forth in SEQ ID NO: 150 (or a variant thereof),
an HCDR3 that comprises the amino acid sequence set forth in SEQ ID NO: 152 (or a variant thereof),
and a light chain variable region comprising
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
   (q)
a heavy chain variable region comprising
an HCDR1 that comprises the amino acid sequence set forth in SEQ ID NO: 124 (or a variant thereof),
an HCDR2 that comprises the amino acid sequence set forth in SEQ ID NO: 126 (or a variant thereof),
an HCDR3 that comprises the amino acid sequence set forth in SEQ ID NO: 128 (or a variant thereof),
and a light chain variable region comprising
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
   (r)
a heavy chain variable region comprising
an HCDR1 that comprises the amino acid sequence set forth in SEQ ID NO: 148 (or a variant thereof),
an HCDR2 that comprises the amino acid sequence set forth in SEQ ID NO: 150 (or a variant thereof),
an HCDR3 that comprises the amino acid sequence set forth in SEQ ID NO: 152 (or a variant thereof),
and a light chain variable region comprising
an LCDR1 that comprises the amino acid sequence set forth in SEQ ID NO: 116 (or a variant thereof),
an LCDR2 that comprises the amino acid sequence set forth in SEQ ID NO: 118 (or a variant thereof),
an LCDR3 that comprises the amino acid sequence set forth in SEQ ID NO: 120 (or a variant thereof);
   (s)
a heavy chain variable region comprising
an HCDR1 that comprises the amino acid sequence set forth in SEQ ID NO: 148 (or a variant thereof),
an HCDR2 that comprises the amino acid sequence set forth in SEQ ID NO: 1 50 (or a variant thereof),
an HCDR3 that comprises the amino acid sequence set forth in SEQ ID NO: 152 (or a variant thereof),
and a light chain variable region comprising
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
   (t)
a heavy chain variable region comprising
an HCDR1 that comprises the amino acid sequence set forth in SEQ ID NO: 140 (or a variant thereof),
an HCDR2 that comprises the amino acid sequence set forth in SEQ ID NO: 142 (or a variant thereof),
an HCDR3 that comprises the amino acid sequence set forth in SEQ ID NO: 144 (or a variant thereof),
and a light chain variable region comprising
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
   (u)
a heavy chain variable region comprising
an HCDR1 that comprises the amino acid sequence set forth in SEQ ID NO: 156 (or a variant thereof),
an HCDR2 that comprises the amino acid sequence set forth in SEQ ID NO: 158 (or a variant thereof),
an HCDR3 that comprises the amino acid sequence set forth in SEQ ID NO: 160 (or a variant thereof),
and a light chain variable region comprising
an LCDR1 that comprises the amino acid sequence set forth in SEQ ID NO: 164 (or a variant thereof),
an LCDR2 that comprises the amino acid sequence set forth in SEQ ID NO: 166 (or a variant thereof),
an LCDR3 that comprises the amino acid sequence set forth in SEQ ID NO: 168 (or a variant thereof);
   (v)
a heavy chain variable region comprising
an HCDR1 that comprises the amino acid sequence set forth in SEQ ID NO: 172 (or a variant thereof),
an HCDR2 that comprises the amino acid sequence set forth in SEQ ID NO: 174 (or a variant thereof),
an HCDR3 that comprises the amino acid sequence set forth in SEQ ID NO: 176 (or a variant thereof),
and a light chain variable region comprising
an LCDR1 that comprises the amino acid sequence set forth in SEQ ID NO: 180 (or a variant thereof),
an LCDR2 that comprises the amino acid sequence set forth in SEQ ID NO: 182 (or a variant thereof),
an LCDR3 that comprises the amino acid sequence set forth in SEQ ID NO: 184 (or a variant thereof);
   (w)
a heavy chain variable region comprising
an HCDR1 that comprises the amino acid sequence set forth in SEQ ID NO: 188 (or a variant thereof),
an HCDR2 that comprises the amino acid sequence set forth in SEQ ID NO: 190 (or a variant thereof),
an HCDR3 that comprises the amino acid sequence set forth in SEQ ID NO: 192 (or a variant thereof),
and a light chain variable region comprising
an LCDR1 that comprises the amino acid sequence set forth in SEQ ID NO: 196 (or a variant thereof),
an LCDR2 that comprises the amino acid sequence set forth in SEQ ID NO: 198 (or a variant thereof),
an LCDR3 that comprises the amino acid sequence set forth in SEQ ID NO: 200 (or a variant thereof);
   (x)
a heavy chain variable region comprising
an HCDR1 that comprises the amino acid sequence set forth in SEQ ID NO: 204 (or a variant thereof),
an HCDR2 that comprises the amino acid sequence set forth in SEQ ID NO: 206 (or a variant thereof),
an HCDR3 that comprises the amino acid sequence set forth in SEQ ID NO: 208 (or a variant thereof),
and a light chain variable region comprising
an LCDR1 that comprises the amino acid sequence set forth in SEQ ID NO: 212 (or a variant thereof),
an LCDR2 that comprises the amino acid sequence set forth in SEQ ID NO: 214 (or a variant thereof),
an LCDR3 that comprises the amino acid sequence set forth in SEQ ID NO: 216 (or a variant thereof);
   (y)
a heavy chain variable region comprising
an HCDR1 that comprises the amino acid sequence set forth in SEQ ID NO: 220 (or a variant thereof),
an HCDR2 that comprises the amino acid sequence set forth in SEQ ID NO: 222 (or a variant thereof),
an HCDR3 that comprises the amino acid sequence set forth in SEQ ID NO: 224 (or a variant thereof),
and a light chain variable region comprising
an LCDR1 that comprises the amino acid sequence set forth in SEQ ID NO: 228 (or a variant thereof),
an LCDR2 that comprises the amino acid sequence set forth in SEQ ID NO: 230 (or a variant thereof),
an LCDR3 that comprises the amino acid sequence set forth in SEQ ID NO: 232 (or a variant thereof);
   (z)
a heavy chain variable region comprising
an HCDR1 that comprises the amino acid sequence set forth in SEQ ID NO: 236 (or a variant thereof),
an HCDR2 that comprises the amino acid sequence set forth in SEQ ID NO: 238 (or a variant thereof),
an HCDR3 that comprises the amino acid sequence set forth in SEQ ID NO: 240 (or a variant thereof),
and a light chain variable region comprising
an LCDR1 that comprises the amino acid sequence set forth in SEQ ID NO: 244 (or a variant thereof),
an LCDR2 that comprises the amino acid sequence set forth in SEQ ID NO: 246 (or a variant thereof),
an LCDR3 that comprises the amino acid sequence set forth in SEQ ID NO: 248 (or a variant thereof);
   (aa)
a heavy chain variable region comprising
an HCDR1 that comprises the amino acid sequence set forth in SEQ ID NO: 252 (or a variant thereof),
an HCDR2 that comprises the amino acid sequence set forth in SEQ ID NO: 254 (or a variant thereof),
an HCDR3 that comprises the amino acid sequence set forth in SEQ ID NO: 256 (or a variant thereof),
and a light chain variable region comprising
an LCDR1 that comprises the amino acid sequence set forth in SEQ ID NO: 260 (or a variant thereof),
an LCDR2 that comprises the amino acid sequence set forth in SEQ ID NO: 262 (or a variant thereof),
an LCDR3 that comprises the amino acid sequence set forth in SEQ ID NO: 264 (or a variant thereof);
   (ab)
a heavy chain variable region comprising
an HCDR1 that comprises the amino acid sequence set forth in SEQ ID NO: 268 (or a variant thereof),
an HCDR2 that comprises the amino acid sequence set forth in SEQ ID NO: 270 (or a variant thereof),
an HCDR3 that comprises the amino acid sequence set forth in SEQ ID NO: 272 (or a variant thereof),
and a light chain variable region comprising
an LCDR1 that comprises the amino acid sequence set forth in SEQ ID NO: 260 (or a variant thereof),
an LCDR2 that comprises the amino acid sequence set forth in SEQ ID NO: 262 (or a variant thereof),
an LCDR3 that comprises the amino acid sequence set forth in SEQ ID NO: 264 (or a variant thereof);
   (ac)
a heavy chain variable region comprising
an HCDR1 that comprises the amino acid sequence set forth in SEQ ID NO: 276 (or a variant thereof),
an HCDR2 that comprises the amino acid sequence set forth in SEQ ID NO: 278 (or a variant thereof),
an HCDR3 that comprises the amino acid sequence set forth in SEQ ID NO: 280 (or a variant thereof),
and a light chain variable region comprising
an LCDR1 that comprises the amino acid sequence set forth in SEQ ID NO: 284 (or a variant thereof),
an LCDR2 that comprises the amino acid sequence set forth in SEQ ID NO: 286 (or a variant thereof),
an LCDR3 that comprises the amino acid sequence set forth in SEQ ID NO: 288 (or a variant thereof);
   (ad)
a heavy chain variable region comprising
an HCDR1 that comprises the amino acid sequence set forth in SEQ ID NO: 292 (or a variant thereof),
an HCDR2 that comprises the amino acid sequence set forth in SEQ ID NO: 294 (or a variant thereof),
an HCDR3 that comprises the amino acid sequence set forth in SEQ ID NO: 296 (or a variant thereof),
and a light chain variable region comprising
an LCDR1 that comprises the amino acid sequence set forth in SEQ ID NO: 300 (or a variant thereof),
an LCDR2 that comprises the amino acid sequence set forth in SEQ ID NO: 302 (or a variant thereof),
an LCDR3 that comprises the amino acid sequence set forth in SEQ ID NO: 304 (or a variant thereof);
   (ae)
a heavy chain variable region comprising
an HCDR1 that comprises the amino acid sequence set forth in SEQ ID NO: 308 (or a variant thereof),
an HCDR2 that comprises the amino acid sequence set forth in SEQ ID NO: 310 (or a variant thereof),
an HCDR3 that comprises the amino acid sequence set forth in SEQ ID NO: 312 (or a variant thereof),
and a light chain variable region comprising
an LCDR1 that comprises the amino acid sequence set forth in SEQ ID NO: 316 (or a variant thereof),
an LCDR2 that comprises the amino acid sequence set forth in SEQ ID NO: 318 (or a variant thereof),
an LCDR3 that comprises the amino acid sequence set forth in SEQ ID NO: 320 (or a variant thereof);
   (af)
a heavy chain variable region comprising
an HCDR1 that comprises the amino acid sequence set forth in SEQ ID NO: 324 (or a variant thereof),
an HCDR2 that comprises the amino acid sequence set forth in SEQ ID NO: 326 (or a variant thereof),
an HCDR3 that comprises the amino acid sequence set forth in SEQ ID NO: 328 (or a variant thereof),
and a light chain variable region comprising
an LCDR1 that comprises the amino acid sequence set forth in SEQ ID NO: 332 (or a variant thereof),
an LCDR2 that comprises the amino acid sequence set forth in SEQ ID NO: 334 (or a variant thereof),
an LCDR3 that comprises the amino acid sequence set forth in SEQ ID NO: 336 (or a variant thereof);
and/or
   (ag)
a heavy chain variable region comprising
an HCDR1 that comprises the amino acid sequence set forth in SEQ ID NO: 340 (or a variant thereof),
an HCDR2 that comprises the amino acid sequence set forth in SEQ ID NO: 342 (or a variant thereof),
an HCDR3 that comprises the amino acid sequence set forth in SEQ ID NO: 344 (or a variant thereof),
and a light chain variable region comprising
an LCDR1 that comprises the amino acid sequence set forth in SEQ ID NO: 348 (or a variant thereof),
an LCDR2 that comprises the amino acid sequence set forth in SEQ ID NO: 350 (or a variant thereof),
an LCDR3 that comprises the amino acid sequence set forth in SEQ ID NO: 352 (or a variant thereof).

In an embodiment of the disclosure, an antibody or antigen-binding fragment thereof that binds specifically to C5, which is in a co-formulation of the present disclosure comprises:

(i)
a heavy chain variable region that comprises the amino acid sequence set forth in SEQ ID NO: 2 (or a variant thereof),
and
a light chain variable region that comprises the amino acid sequence set forth in SEQ ID NO: 10 (or a variant thereof);
   (ii)
a heavy chain variable region that comprises the amino acid sequence set forth in SEQ ID NO: 18 (or a variant thereof),
and
a light chain variable region that comprises the amino acid sequence set forth in SEQ ID NO: 26 (or a variant thereof);
   (iii)
a heavy chain variable region that comprises the amino acid sequence set forth in SEQ ID NO: 34 (or a variant thereof),
and
a light chain variable region that comprises the amino acid sequence set forth in SEQ ID NO: 42 (or a variant thereof);
   (iv)
a heavy chain variable region that comprises the amino acid sequence set forth in SEQ ID NO: 50 (or a variant thereof),
and
a light chain variable region that comprises the amino acid sequence set forth in SEQ ID NO: 58 (or a variant thereof);
   (v)
a heavy chain variable region that comprises the amino acid sequence set forth in SEQ ID NO: 66 (or a variant thereof),
and
a light chain variable region that comprises the amino acid sequence set forth in SEQ ID NO: 74 (or a variant thereof);
   (vi)
a heavy chain variable region that comprises the amino acid sequence set forth in SEQ ID NO: 82 (or a variant thereof),
and
a light chain variable region that comprises the amino acid sequence set forth in SEQ ID NO: 90 (or a variant thereof);
   (vii)
a heavy chain variable region that comprises the amino acid sequence set forth in SEQ ID NO: 98 (or a variant thereof),
and
a light chain variable region that comprises the amino acid sequence set forth in SEQ ID NO: 106 (or a variant thereof);
   (viii)
a heavy chain variable region that comprises the amino acid sequence set forth in SEQ ID NO: 98 (or a variant thereof),
and
a light chain variable region that comprises the amino acid sequence set forth in SEQ ID NO: 114 (or a variant thereof);
   (ix)
a heavy chain variable region that comprises the amino acid sequence set forth in SEQ ID NO: 122 (or a variant thereof),
and
a light chain variable region that comprises the amino acid sequence set forth in SEQ ID NO: 106 (or a variant thereof);
   (x)
a heavy chain variable region that comprises the amino acid sequence set forth in SEQ ID NO: 98 (or a variant thereof),
and
a light chain variable region that comprises the amino acid sequence set forth in SEQ ID NO: 130 (or a variant thereof);
   (xi)
a heavy chain variable region that comprises the amino acid sequence set forth in SEQ ID NO: 138 (or a variant thereof),
and
a light chain variable region that comprises the amino acid sequence set forth in SEQ ID NO: 106 (or a variant thereof);
   (xii)
a heavy chain variable region that comprises the amino acid sequence set forth in SEQ ID NO: 146 (or a variant thereof),
and
a light chain variable region that comprises the amino acid sequence set forth in SEQ ID NO: 106 (or a variant thereof);
   (xiii)
a heavy chain variable region that comprises the amino acid sequence set forth in SEQ ID NO: 122 (or a variant thereof),
and
a light chain variable region that comprises the amino acid sequence set forth in SEQ ID NO: 130 (or a variant thereof);
   (xiv)
a heavy chain variable region that comprises the amino acid sequence set forth in SEQ ID NO: 146 (or a variant thereof),
and
a light chain variable region that comprises the amino acid sequence set forth in SEQ ID NO: 114 (or a variant thereof);
   (xv)
a heavy chain variable region that comprises the amino acid sequence set forth in SEQ ID NO: 146 (or a variant thereof),
and
a light chain variable region that comprises the amino acid sequence set forth in SEQ ID NO: 130 (or a variant thereof);
   (xvi)
a heavy chain variable region that comprises the amino acid sequence set forth in SEQ ID NO: 138 (or a variant thereof),
and
a light chain variable region that comprises the amino acid sequence set forth in SEQ ID NO: 130 (or a variant thereof);
   (xvii)
a heavy chain variable region that comprises the amino acid sequence set forth in SEQ ID NO: 154 (or a variant thereof),
and
a light chain variable region that comprises the amino acid sequence set forth in SEQ ID NO: 162 (or a variant thereof);
   (xviii)
a heavy chain variable region that comprises the amino acid sequence set forth in SEQ ID NO: 170 (or a variant thereof),
and
a light chain variable region that comprises the amino acid sequence set forth in SEQ ID NO: 178 (or a variant thereof);
   (xix)
a heavy chain variable region that comprises the amino acid sequence set forth in SEQ ID NO: 186 (or a variant thereof),
and
a light chain variable region that comprises the amino acid sequence set forth in SEQ ID NO: 194 (or a variant thereof);
   (xx)
a heavy chain variable region that comprises the amino acid sequence set forth in SEQ ID NO: 202 (or a variant thereof),
and
a light chain variable region that comprises the amino acid sequence set forth in SEQ ID NO: 210 (or a variant thereof);
   (xxi)
a heavy chain variable region that comprises the amino acid sequence set forth in SEQ ID NO: 218 (or a variant thereof),
and
a light chain variable region that comprises the amino acid sequence set forth in SEQ ID NO: 226 (or a variant thereof);
   (xxii)
a heavy chain variable region that comprises the amino acid sequence set forth in SEQ ID NO: 234 (or a variant thereof),
and
a light chain variable region that comprises the amino acid sequence set forth in SEQ ID NO: 242 (or a variant thereof);
   (xxiii)
a heavy chain variable region that comprises the amino acid sequence set forth in SEQ ID NO: 250 (or a variant thereof),
and
a light chain variable region that comprises the amino acid sequence set forth in SEQ ID NO: 258 (or a variant thereof);
   (xxiv)
a heavy chain variable region that comprises the amino acid sequence set forth in SEQ ID NO: 266 (or a variant thereof),
and
a light chain variable region that comprises the amino acid sequence set forth in SEQ ID NO: 258 (or a variant thereof);
   (xxv)
a heavy chain variable region that comprises the amino acid sequence set forth in SEQ ID NO: 274 (or a variant thereof),
and
a light chain variable region that comprises the amino acid sequence set forth in SEQ ID NO: 282 (or a variant thereof);
   (xxvi)
a heavy chain variable region that comprises the amino acid sequence set forth in SEQ ID NO: 290 (or a variant thereof),
and
a light chain variable region that comprises the amino acid sequence set forth in SEQ ID NO: 298 (or a variant thereof);
   (xxvii)
a heavy chain variable region that comprises the amino acid sequence set forth in SEQ ID NO: 306 (or a variant thereof),
and
a light chain variable region that comprises the amino acid sequence set forth in SEQ ID NO: 314 (or a variant thereof);
   (xxviii)
a heavy chain variable region that comprises the amino acid sequence set forth in SEQ ID NO: 322 (or a variant thereof),
and
a light chain variable region that comprises the amino acid sequence set forth in SEQ ID NO: 330 (or a variant thereof);
and/or
   (xxix)
a heavy chain variable region that comprises the amino acid sequence set forth in SEQ ID NO: 338 (or a variant thereof),
and
a light chain variable region that comprises the amino acid sequence set forth in SEQ ID NO: 346 (or a variant thereof).

In an embodiment of the disclosure, an antibody or antigen-binding fragment thereof that binds specifically to C5, which is in a co-formulation of the present disclosure comprises a heavy chain comprising the amino acid sequence:

(SEQ ID NO: 358)
QVQLQESGPGLVKPSETLSLTCTVSGDSVSSSYWTWIRQP
PGKGLEWIGYIYYSGSSNYNPSLKSRATISVDTSKNQFSL
KLSSVTAADTAVYYCAREGNVDTTMIFDYWGQGTLVTVSS
ASTKGPSVFPLAPCSRSTSESTAALGCLVKDYFPEPVTVS
WNSGALTSGVHTFPAVLQSSGLYSLSSVVTVPSSSLGTKT
YTCNVDHKPSNTKVDKRVESKYGPPCPPCPAPEFLGGPSV
FLFPPKPKDTLMISRTPEVTCVVVDVSQEDPEVQFNWYVD
GVEVHNAKTKPREEQFNSTYRVVSVLTVLHQDWLNGKEYK
CKVSNKGLPSSIEKTISKAKGQPREPQVYTLPPSQEEMTK
NQVSLTCLVKGFYPSDIAVEWESNGQPENNYKTTPPVLDS
DGSFFLYSRLTVDKSRWQEGNVFSCSVMHEALHNHYTQKS
LSLSLGK

a light chain comprising the amino acid sequence:

(SEQ ID NO: 359)
AIQMTQSPSSLSASVGDRVTITCRASQGIRNDLGWYQQKP
GKAPKLLIYAASSLQSGVPSRFAGRGSGTDFTLTISSLQP
EDFATYYCLQDENYPWTFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSENRGEC;

such an antibody may be referred to herein as Pozelimab or REGN3918 (variable regions and CDRs underscored).

“H2M11683N”; “H2M11686N”; “H4H12159P”; “H4H12161P”; “H4H12163P”; “H4H12164P”; “H4H12166P”; “H4H12166P2”; “H4H12166P3”; “H4H12166P4”; “H4H12166P5”; “H4H12166P6”; “H4H12166P7”; “H4H12166P8”; “H4H12166P9”; “H4H12166P10”; “H4H12167P”; “H4H12168P”; “H4H12169P”; “H4H12170P”; “H4H12171P”; “H4H12175P”; “H4H12176P2”; “H4H12177P2”; “H4H12183P2”; “H2M 11682N”; “H2M11684N”; “H2M 11694N” or “H2M 11695N”, unless otherwise stated, refer to anti-C5 antigen-binding proteins, e.g., antibodies and antigen-binding fragments thereof (including multispecific antigen-binding proteins), that bind specifically to C5, comprising an immunoglobulin heavy chain or variable region thereof (V_{H}) comprising the amino acid sequence specifically set forth herein corresponding, in Table A herein or Table 1 of WO2017/218515 (and the sequences set forth therein), to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 2; 18; 34; 50; 66; 82; 98; 98; 122; 98; 138; 146; 122; 146; 146; 138; 154; 170; 186; 202; 218; 234; 250; 266; 274; 290; 306; 322 or 338) (or a variant thereof), and/or an immunoglobulin light chain or variable region thereof (V_{L}) comprising the amino acid sequence specifically set forth herein corresponding, in Table A herein or Table 1 of WO2017/218515 (and the sequences set forth therein), to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 10; 26; 42; 58; 74; 90; 106; 114; 106; 130; 106; 106; 130; 114; 130; 130; 162; 178; 194; 210; 226; 242; 258; 258; 282; 298; 314; 330 or 346) (or a variant thereof); and/or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)). In an embodiment of the disclosure, the V_{H} is linked to an IgG constant heavy chain domain (e.g., IgG1 or IgG4 (e.g., IgG4 (S228P mutant))) and/or the V_{L} is linked to a lambda or kappa constant light chain domain.

An “anti-C5” antibody or antigen-binding fragment or antibody or antigen-binding fragment that “binds specifically” to C5 binds to human C5 with a K_{D} of at least 1 nM (i.e., 1 nM or a higher affinity), e.g., about 0.1 or 0.2 nM.

In an embodiment of the invention, an anti-C5 antibody or antigen-binding fragment is missing the C-terminal Lysine from the heavy chain.

### Interfering RNA (iRNA)

The present disclosure provides a co-formulation that includes an anti-C5 antibody or antigen-binding fragment thereof (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; crovalimab; Eculizumab, tesidolumab, mubodina or Ravulizumab; preferably, Pozelimab); and an iRNA which effects the RNA-induced silencing complex (RISC)-mediated cleavage of RNA transcripts of a C5 gene (C5 iRNA), e.g. Cemdisiran (e.g., Cemdisiran/Pozelimab). The C5 gene may be within a cell, e.g., a cell within a subject, such as a human. The present disclosure provides iRNA agents for inclusion in a co-formulation of the disclosure which effect the RNA-induced silencing complex (RISC)-mediated cleavage of RNA transcripts of a complement component C5 gene.

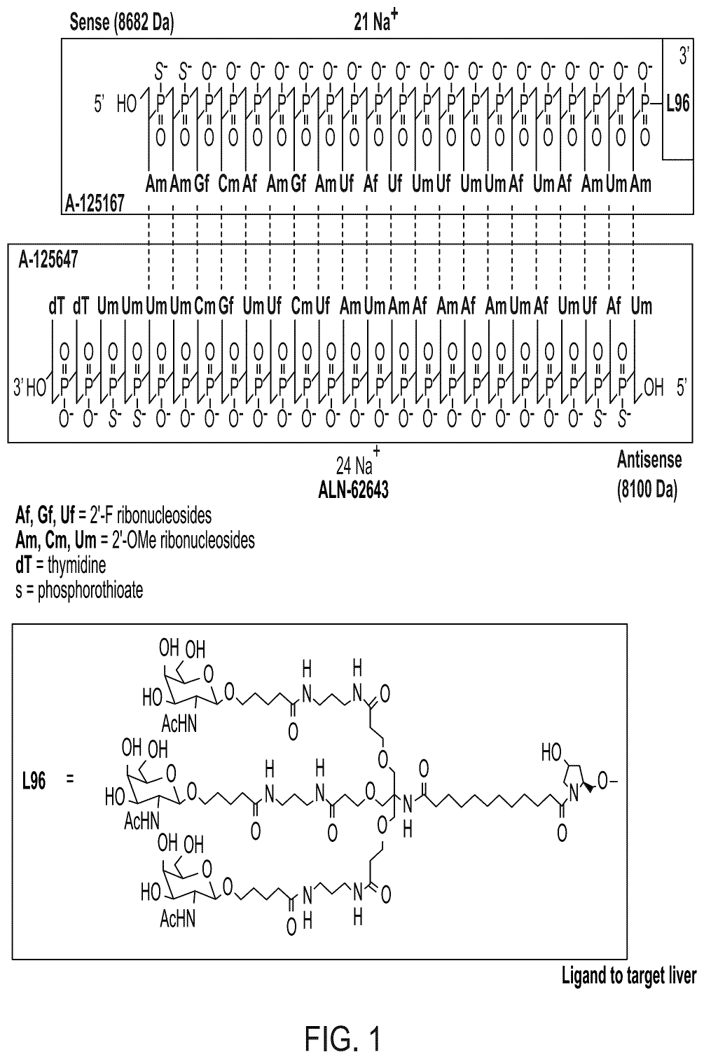

Cemdisiran is a chemically synthesized double-stranded oligonucleotide glycoconjugate that is covalently linked to a ligand containing 3 GalNAc residues to facilitate targeted delivery to the liver. See e.g., FIG. 1. All nucleosides are modified with 2′-deoxy, 2′-methoxy, or 2′-fluoro groups and are connected through 3′ to 5′ phosphodiester linkages, thus forming the sugar-phosphate backbone of the oligonucleotide.

The sense strand (A-125167) contains 21 nucleotides and the antisense strand (A-125647) contains 25. The 3′-end of the sense strand is conjugated to a triantennary GalNAc moiety (referred to as L96) through a phosphodiester linkage.

The antisense strand (A-125647) contains four phosphorothioate linkages, two consecutive phosphorothioate linkages at the 3′ end and two at the 5′ end. The sense strand (A-125167) contains two phosphorothioate linkages at the 5′ end. The 21 nucleotides of the sense strand hybridize with the complementary 21 nucleotides of the antisense strand, thus forming 21 nucleotide base pairs duplex with a 4-base overhang at the 3′-end of the antisense strand. The bases involved in base pair formation are connected with a center dot. Cemdisiran is preferably in a salt form, e.g., the Na⁺ salt form, but the present disclosure includes embodiments including Cemdisiran in the free acid form as well as in other salt forms, e.g., Ca²⁺ salts.

When expressing, herein, the concentration of RNAi in a composition, such as a co-formulation of the present disclosure, in terms of mass per volume (e.g., mg/ml), the RNAi is in a salt form or a free acid form. Preferably, when referring to Cemdisiran as such, the Cemdisiran is in salt form, preferably Na⁺ salt form. Na⁺ counter-ions are present due to the net-negatively charged ribonucleotide phosphate backbone. The quantity of Cemdisiran free acid form can be obtained by multiplying the Cemdisiran Na⁺ salt form concentration by 0.9443.

The structure of Cemdisiran Sodium (ALN-62643) is shown below wherein A-125167 is on top (5′-3′) and A-125647 is on bottom (3′-5′):

```
                                                                                                                                                                    3'
                    5' Ams-Ams-Gf-Cm-Af-Am-Gf-Am-Uf-Af-Uf-Um-Uf-Um-Um-Af-Um-Af-Am-Um-Am-L96
                            • • • • • • • • • • • • • • • • • • • • •
dT-dT-Ums-Ums-Um-Um-Cm-Gf-Um-Uf-Cm-Uf-Am-Um-Am-Af-Am-Af-Am-Um-Af-Um-Ufs-Afs-Um
3' 5'
```

Af, Gf and Uf=2′-F ribonucleosides
Am, Cm and Um-2′-OMe ribonucleosides
dT=thymidine
S=phosphorothioate
L96 is

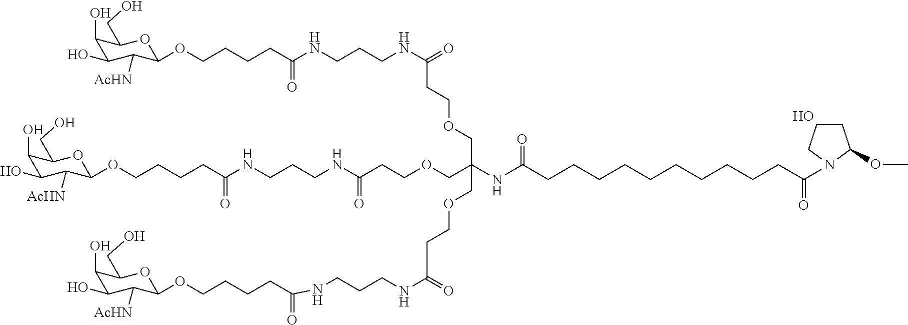

The C5 iRNAs that can be included in co-formulations of the disclosure include an RNA strand (e.g., the antisense strand) having a region which is about 30 nucleotides or less in length, e.g., at least 15, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of a C5 gene.

In an embodiment of the disclosure, a C5 iRNA is a glycoconjugate that includes a double stranded RNA complementary to a region of C5 which is conjugated (e.g., by a linker) to a terminal mono-, or bi-, tri-antennary N-acetylgalactosamine (GalNAc) group, preferably triantennary N-acetylgalactosamine.

In an embodiment of the disclosure, an iRNA agent which may be included in a co-formulation of the present disclosure, includes a single stranded RNA that interacts with a target RNA sequence, e.g., a C5 target mRNA sequence, to direct the cleavage of the target RNA. Without wishing to be bound by theory, it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a single stranded RNA (siRNA) generated within a cell and which promotes the formation of a RISC complex to effect silencing of the target gene, i.e., a C5 gene. Accordingly, the term “siRNA” is also used herein to refer to an iRNA as described above.

In another embodiment, the iRNA agent which may be included in a co-formulation of the present disclosure may be a single-stranded siRNA that is introduced into a cell or organism to inhibit a target mRNA. Single-stranded iRNA agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, an iRNA for use in the compositions, uses, and methods of the disclosure is a double-stranded RNA and is referred to herein as a “double stranded iRNA agent,” “double-stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a C5 gene. In some embodiments of the disclosure, a double-stranded RNA (dsRNA) triggers the degradation of a target RNA, e.g., an mRNA, through a post-transcriptional gene-silencing mechanism referred to herein as RNA interference or iRNA.

In an embodiment of the disclosure, the iRNA is a double-stranded ribonucleic acid (dsRNA) wherein the dsRNA comprises a sense strand and an antisense strand, wherein the sense strand comprises nucleotides (e.g., at least 15 contiguous nucleotides) differing by no more than 3 nucleotides from the nucleotide sequence of C5 (open reading frame underscored):

| | |
|---|---|
| tatatccgtg gtttcctgct acctccaacc atgggccttt tgggaatact ttgtttttta | 60 |
| atcttcctgg ggaaaacctg gggacaggag caaacatatg tcatttcagc accaaaaata | 120 |
| ttccgtgttg gagcatctga aaatattgtg attcaagttt atggatacac tgaagcattt | 180 |
| gatgcaacaa tctctattaa aagttatcct gataaaaaat ttagttactc ctcaggccat | 240 |
| gttcatttat cctcagagaa taaattccaa aactctgcaa tcttaacaat acaaccaaaa | 300 |
| caattgcctg gaggacaaaa cccagtttct tatgtgtatt tggaagttgt atcaaagcat | 360 |
| ttttcaaaat caaaaagaat gccaataacc tatgacaatg gatttctctt cattcataca | 420 |
| gacaaacctg tttatactcc agaccagtca gtaaaagtta gagtttattc gttgaatgac | 480 |
| gacttgaagc cagccaaaag agaaactgtc ttaactttca tagatcctga aggatcagaa | 540 |
| gttgacatgg tagaagaaat tgatcatatt ggaattatct cttttcctga cttcaagatt | 600 |
| ccgtctaatc ctagatatgg tatgtggacg atcaaggcta aatataaaga ggacttttca | 660 |
| acaactggaa ccgcatattt tgaagttaaa gaatatgtct tgccacattt ttctgtctca | 720 |
| atcgagccag aatataattt cattggttac aagaacttta agaattttga aattactata | 780 |
| aaagcaagat atttttataa taaagtagtc actgaggctg acgtttatat cacatttgga | 840 |
| ataagagaag acttaaaaga tgatcaaaaa gaaatgatgc aaacagcaat gcaaaacaca | 900 |
| atgttgataa atggaattgc tcaagtcaca tttgattctg aaacagcagt caaagaactg | 960 |
| tcatactaca gtttagaaga tttaaacaac aagtaccttt atattgctgt aacagtcata | 1020 |
| gagtctacag gtggattttc tgaagaggca gaaatacctg gcatcaaata tgtcctctct | 1080 |
| ccctacaaac tgaatttggt tgctactcct cttttcctga agcctgggat tccatatccc | 1140 |
| atcaaggtgc aggttaaaga ttcgcttgac cagttggtag gaggagtccc agtaacactg | 1200 |
| aatgcacaaa caattgatgt aaaccaagag acatctgact tggatccaag caaaagtgta | 1260 |
| acacgtgttg atgatggagt agcttccttt gtgcttaatc tcccatctgg agtgacggtg | 1320 |
| ctggagttta atgtcaaaac tgatgctcca gatcttccag aagaaaatca ggccagggaa | 1380 |
| ggttaccgag caatagcata ctcatctctc agccaaagtt acctttatat tgattggact | 1440 |
| gataaccata aggctttgct agtgggagaa catctgaata ttattgttac ccccaaaagc | 1500 |
| ccatatattg acaaaataac tcactataat tacttgattt tatccaaggg caaaattatc | 1560 |
| cactttggca cgagggagaa attttcagat gcatcttatc aaagtataaa cattccagta | 1620 |
| acacagaaca tggttccttc atcccgactt ctggtctatt acatcgtcac aggagaacag | 1680 |
| acagcagaat tagtgtctga ttcagtctgg ttaaatattg aagaaaaatg tggcaaccag | 1740 |
| ctccaggttc atctgtctcc tgatgcagat gcatattctc caggccaaac tgtgtctctt | 1800 |
| aatatggcaa ctggaatgga ttcctgggtg gcattagcag cagtggacag tgctgtgtat | 1860 |
| ggagtccaaa gaggagccaa aaagcccttg gaaagagtat ttcaattctt agagaagagt | 1920 |
| gatctgggct gtggggcagg tggtggcctc aacaatgcca atgtgttcca cctagctgga | 1980 |
| cttaccttcc tcactaatgc aaatgcagat gactcccaag aaaatgatga accttgtaaa | 2040 |
| gaaattctca ggccaagaag aacgctgcaa aagaagatag aagaaatagc tgctaaatat | 2100 |
| aaacattcag tagtgaagaa atgttgttac gatggagcct gcgttaataa tgatgaaacc | 2160 |
| tgtgagcagc gagctgcacg gattagttta gggccaagat gcatcaaagc tttcactgaa | 2220 |
| tgttgtgtcg tcgcaagcca gctccgtgct aatatctctc ataaagacat gcaattggga | 2280 |
| aggctacaca tgaagaccct gttaccagta agcaagccag aaattcggag ttattttcca | 2340 |
| gaaagctggt tgtgggaagt tcatcttgtt cccagaagaa aacagttgca gtttgcccta | 2400 |
| cctgattctc taaccacctg ggaaattcaa ggcgttggca tttcaaacac tggtatatgt | 2460 |
| gttgctgata ctgtcaaggc aaaggtgttc aaagatgtct tcctggaaat gaatatacca | 2520 |
| tattctgttg tacgaggaga acagatccaa ttgaaaggaa ctgtttacaa ctataggact | 2580 |
| tctgggatgc agttctgtgt taaaatgtct gctgtggagg gaatctgcac ttcggaaagc | 2640 |
| ccagtcattg atcatcaggg cacaaagtcc tccaaatgtg tgcgccagaa agtagagggc | 2700 |
| tcctccagtc acttggtgac attcactgtg cttcctctgg aaattggcct tcacaacatc | 2760 |
| aatttttcac tggagacttg gtttggaaaa gaaatcttag taaaaacatt acgagtggtg | 2820 |
| ccagaaggtg tcaaaaggga aagctattct ggtgttactt tggatcctag gggtatttat | 2880 |
| ggtaccatta gcagacgaaa ggagttccca tacaggatac ccttagattt ggtccccaaa | 2940 |
| acagaaatca aaaggatttt gagtgtaaaa ggactgcttg taggtgagat cttgtctgca | 3000 |
| gttctaagtc aggaaggcat caatatccta acccacctcc ccaaagggag tgcagaggcg | 3060 |
| gagctgatga gcgttgtccc agtattctat gtttttcact acctggaaac aggaaatcat | 3120 |
| tggaacattt ttcattctga cccattaatt gaaaagcaga aactgaagaa aaaattaaaa | 3180 |
| gaagggatgt tgagcattat gtcctacaga aatgctgact actcttacag tgtgtggaag | 3240 |
| ggtggaagtg ctagcacttg gttaacagct tttgctttaa gagtacttgg acaagtaaat | 3300 |
| aaatacgtag agcagaacca aaattcaatt tgtaattctt tattgtggct agttgagaat | 3360 |
| tatcaattag ataatggatc tttcaaggaa aattcacagt atcaaccaat aaaattacag | 3420 |
| ggtaccttgc ctgttgaagc ccgagagaac agcttatatc ttacagcctt tactgtgatt | 3480 |
| ggaattagaa aggctttcga tatatgcccc ctggtgaaaa tcgacacagc tctaattaaa | 3540 |
| gctgacaact ttctgcttga aaatacactg ccagcccaga gcacctttac attggccatt | 3600 |
| tctgcgtatg ctctttccct gggagataaa actcacccac agtttcgttc aattgtttca | 3660 |
| gctttgaaga gagaagcttt ggttaaaggt aatccaccca tttatcgttt ttggaaagac | 3720 |
| aatcttcagc ataaagacag ctctgtacct aacactggta cggcacgtat ggtagaaaca | 3780 |
| actgcctatg ctttactcac cagtctgaac ttgaaagata taaattatgt taacccagtc | 3840 |
| atcaaatggc tatcagaaga gcagaggtat ggaggtggct tttattcaac ccaggacaca | 3900 |
| atcaatgcca ttgagggcct gacggaatat tcactcctgg ttaaacaact ccgcttgagt | 3960 |
| atggacatcg atgtttctta caagcataaa ggtgccttac ataattataa aatgacagac | 4020 |
| aagaatttcc ttgggaggcc agtagaggtg cttctcaatg atgacctcat tgtcagtaca | 4080 |
| ggatttggca gtggcttggc tacagtacat gtaacaactg tagttcacaa aaccagtacc | 4140 |
| tctgaggaag tttgcagctt ttatttgaaa atcgatactc aggatattga agcatcccac | 4200 |
| tacagaggct acggaaactc tgattacaaa cgcatagtag catgtgccag ctacaagccc | 4260 |
| agcagggaag aatcatcatc tggatcctct catgcggtga tggacatctc cttgcctact | 4320 |
| ggaatcagtg caaatgaaga agacttaaaa gcccttgtgg aaggggtgga tcaactattc | 4380 |
| actgattacc aaatcaaaga tggacatgtt attctgcaac tgaattcgat tccctccagt | 4440 |
| gatttccttt gtgtacgatt ccggatattt gaactctttg aagttgggtt tctcagtcct | 4500 |
| gccactttca cagtgtacga ataccacaga ccagataaac agtgtaccat gttttatagc | 4560 |
| acttccaata tcaaaattca gaaagtctgt gaaggagccg cgtgcaagtg tgtagaagct | 4620 |
| gattgtgggc aaatgcagga agaattggat ctgacaatct ctgcagagac aagaaaacaa | 4680 |
| acagcatgta aaccagagat tgcatatgct tataaagtta gcatcacatc catcactgta | 4620 |
| gaaaatgttt ttgtcaagta caaggcaacc cttctggata tctacaaaac tggggaagct | 4800 |
| gttgctgaga aagactctga gattaccttc attaaaaagg taacctgtac taacgctgag | 4860 |
| ctggtaaaag gaagacagta cttaattatg ggtaaagaag ccctccagat aaaatacaat | 4920 |
| ttcagtttca ggtacatcta ccctttagat tccttgacct ggattgaata ctggcctaga | 4980 |
| gacacaacat gttcatcgtg tcaagcattt ttagctaatt tagatgaatt tgccgaagat | 5040 |
| atctttttaa atggatgcta aaattcctga agttcagctg catacagttt gcacttatgg | 5100 |
| actcctgttg ttgaagttcg tttttttgtt ttcttctttt tttaaacatt catagctggt | 5160 |
| cttatttgta aagctcactt tacttagaat tagtggcact tgcttttatt agagaatgat | 5220 |
| ttcaaatgct gtaactttct gaaataacat ggccttggag ggcatgaaga cagatactcc | 5280 |
| tccaaggtta ttggacaccg gaaacaataa attggaacac ctcctcaaac ctaccactca | 5340 |
| ggaatgtttg ctggggccga aagaacagtc cattgaaagg gagtattaca aaaacatggc | 5400 |
| ctttgcttga aagaaaatac caaggaacag gaaactgatc attaaagcct gagtttgctt | 5460 |
| tcaaaaaaaa aaaaaaaaaa | 5480 |

(SEQ ID NO: 360) and the antisense strand comprises nucleotides (e.g., at least 15 contiguous nucleotides) differing by no more than 3 nucleotides from the nucleotide sequence of:

| (SEQ ID NO: 361) | |
|---|---|
| tttttttttt ttttttttga aagcaaactc aggctttaat gatcagtttc ctgttccttg | 60 |
| gtattttctt tcaagcaaag gccatgtttt tgtaatactc cctttcaatg gactgttctt | 120 |
| tcggccccag caaacattcc tgagtggtag gtttgaggag gtgttccaat ttattgtttc | 180 |
| cggtgtccaa taaccttgga ggagtatctg tcttcatgcc ctccaaggcc atgttatttc | 240 |
| agaaagttac agcatttgaa atcattctct aataaaagca agtgccacta attctaagta | 300 |
| aagtgagctt tacaaataag accagctatg aatgtttaaa aaaagaagaa aacaaaaaaa | 360 |
| cgaacttcaa caacaggagt ccataagtgc aaactgtatg cagctgaact tcaggaattt | 420 |
| tagcatccat ttaaaaagat atcttcggca aattcatcta aattagctaa aaatgcttga | 480 |
| cacgatgaac atgttgtgtc tctaggccag tattcaatcc aggtcaagga atctaaaggg | 540 |
| tagatgtacc tgaaactgaa attgtatttt atctggaggg cttctttacc cataattaag | 600 |
| tactgtcttc cttttaccag ctcagcgtta gtacaggtta cctttttaat gaaggtaatc | 660 |
| tcagagtctt tctcagcaac agcttcccca gttttgtaga tatccagaag ggttgccttg | 720 |
| tacttgacaa aaacattttc tacagtgatg gatgtgatgc taactttata agcatatgca | 780 |
| atctctggtt tacatgctgt ttgttttctt gtctctgcag agattgtcag atccaattct | 840 |
| tcctgcattt gcccacaatc agcttctaca cacttgcacg cggctccttc acagactttc | 900 |
| tgaattttga tattggaagt gctataaaac atggtacact gtttatctgg tctgtggtat | 960 |
| tcgtacactg tgaaagtggc aggactgaga aacccaactt caaagagttc aaatatccgg | 1020 |
| aatcgtacac aaaggaaatc actggaggga atcgaattca gttgcagaat aacatgtcca | 1080 |
| tctttgattt ggtaatcagt gaatagttga tccacccctt ccacaagggc ttttaagtct | 1140 |
| tcttcatttg cactgattcc agtaggcaag gagatgtcca tcaccgcatg agaggatcca | 1200 |
| gatgatgatt cttccctgct gggcttgtag ctggcacatg ctactatgcg tttgtaatca | 1260 |
| gagtttccgt agcctctgta gtgggatgct tcaatatcct gagtatcgat tttcaaataa | 1320 |
| aagctgcaaa cttcctcaga ggtactggtt ttgtgaacta cagttgttac atgtactgta | 1380 |
| gccaagccac tgccaaatcc tgtactgaca atgaggtcat cattgagaag cacctctact | 1440 |
| ggcctcccaa ggaaattctt gtctgtcatt ttataattat gtaaggcacc tttatgcttg | 1500 |
| taagaaacat cgatgtccat actcaagcgg agttgtttaa ccaggagtga atattccgtc | 1560 |
| aggccctcaa tggcattgat tgtgtcctgg gttgaataaa agccacctcc atacctctgc | 1620 |
| tcttctgata gccatttgat gactgggtta acataattta tatctttcaa gttcagactg | 1680 |
| gtgagtaaag cataggcagt tgtttctacc atacgtgccg taccagtgtt aggtacagag | 1740 |
| ctgtctttat gctgaagatt gtctttccaa aaacgataaa tgggtggatt acctttaacc | 1800 |
| aaagcttctc tottcaaagc tgaaacaatt gaacgaaact gtgggtgagt tttatctccc | 1860 |
| agggaaagag catacgcaga aatggccaat gtaaaggtgc tctgggctgg cagtgtattt | 1920 |
| tcaagcagaa agttgtcagc tttaattaga gctgtgtcga ttttcaccag ggggcatata | 1980 |
| tcgaaagcct ttctaattcc aatcacagta aaggctgtaa gatataagct gttctctcgg | 2040 |
| gcttcaacag gcaaggtacc ctgtaatttt attggttgat actgtgaatt ttccttgaaa | 2100 |
| gatccattat ctaattgata attctcaact agccacaata aagaattaca aattgaattt | 2160 |
| tggttctgct ctacgtattt atttacttgt ccaagtactc ttaaagcaaa agctgttaac | 2220 |
| caagtgctag cacttccacc cttccacaca ctgtaagagt agtcagcatt tctgtaggac | 2280 |
| ataatgctca acatcccttc ttttaatttt ttcttcagtt tctgcttttc aattaatggg | 2340 |
| tcagaatgaa aaatgttcca atgatttcct gtttccaggt agtgaaaaac atagaatact | 2400 |
| gggacaacgc tcatcagctc cgcctctgca ctccctttgg ggaggtgggt taggatattg | 2460 |
| atgccttcct gacttagaac tgcagacaag atctcaccta caagcagtcc ttttacactc | 2520 |
| aaaatccttt tgatttctgt tttggggacc aaatctaagg gtatcctgta tgggaactcc | 2580 |
| tttcgtctgc taatggtacc ataaataccc ctaggatcca aagtaacacc agaatagctt | 2640 |
| tcccttttga caccttctgg caccactcgt aatgttttta ctaagatttc ttttccaaac | 2700 |
| caagtctcca gtgaaaaatt gatgttgtga aggccaattt ccagaggaag cacagtgaat | 2760 |
| gtcaccaagt gactggagga gccctctact ttctggcgca cacatttgga ggactttgtg | 2820 |
| ccctgatgat caatgactgg gctttccgaa gtgcagattc cctccacagc agacatttta | 2880 |
| acacagaact gcatcccaga agtcctatag ttgtaaacag ttcctttcaa ttggatctgt | 2940 |
| tctcctcgta caacagaata tggtatattc atttccagga agacatcttt gaacaccttt | 3000 |
| gccttgacag tatcagcaac acatatacca gtgtttgaaa tgccaacgcc ttgaatttcc | 3060 |
| caggtggtta gagaatcagg tagggcaaac tgcaactgtt ttcttctggg aacaagatga | 3120 |
| acttcccaca accagctttc tggaaaataa ctccgaattt ctggcttgct tactggtaac | 3180 |
| agggtcttca tgtgtagcct toccaattgc atgtctttat gagagatatt agcacggagc | 3240 |
| tggcttgcga cgacacaaca ttcagtgaaa gctttgatgc atcttggccc taaactaatc | 3300 |
| cgtgcagctc gctgctcaca ggtttcatca ttattaacgc aggctccatc gtaacaacat | 3360 |
| ttcttcacta ctgaatgttt atatttagca gctatttctt ctatcttctt ttgcagcgtt | 3420 |
| cttcttggcc tgagaatttc tttacaaggt tcatcatttt cttgggagtc atctgcattt | 3480 |
| gcattagtga ggaaggtaag tccagctagg tggaacacat tggcattgtt gaggccacca | 3540 |
| cctgccccac agcccagatc actcttctct aagaattgaa atactctttc caagggcttt | 3600 |
| ttggctcctc tttggactcc atacacagca ctgtccactg ctgctaatgc cacccaggaa | 3660 |
| tccattccag ttgccatatt aagagacaca gtttggcctg gagaatatgc atctgcatca | 3720 |
| ggagacagat gaacctggag ctggttgcca catttttctt caatatttaa ccagactgaa | 3780 |
| tcagacacta attctgctgt ctgttctcct gtgacgatgt aatagaccag aagtcgggat | 3840 |
| gaaggaacca tgttctgtgt tactggaatg tttatacttt gataagatgc atctgaaaat | 3900 |
| ttctccctcg tgccaaagtg gataattttg cccttggata aaatcaagta attatagtga | 3960 |
| gttattttgt caatatatgg gcttttgggg gtaacaataa tattcagatg ttctcccact | 4020 |
| agcaaagcct tatggttatc agtccaatca atataaaggt aactttggct gagagatgag | 4080 |
| tatgctattg ctcggtaacc ttccctggcc tgattttctt ctggaagatc tggagcatca | 4140 |
| gttttgacat taaactccag caccgtcact ccagatggga gattaagcac aaaggaagct | 4200 |
| actccatcat caacacgtgt tacacttttg cttggatcca agtcagatgt ctcttggttt | 4260 |
| acatcaattg tttgtgcatt cagtgttact gggactcctc ctaccaactg gtcaagcgaa | 4320 |
| tctttaacct gcaccttgat gggatatgga atcccaggct tcaggaaaag aggagtagca | 4380 |
| accaaattca gtttgtaggg agagaggaca tatttgatgc caggtatttc tgcctcttca | 4440 |
| gaaaatccac ctgtagactc tatgactgtt acagcaatat aaaggtactt gttgtttaaa | 4500 |
| tcttctaaac tgtagtatga cagttctttg actgctgttt cagaatcaaa tgtgacttga | 4560 |
| gcaattccat ttatcaacat tgtgttttgc attgctgttt gcatcatttc tttttgatca | 4620 |
| tcttttaagt cttctcttat tccaaatgtg atataaacgt cagcctcagt gactacttta | 4680 |
| ttataaaaat atcttgcttt tatagtaatt tcaaaattct taaagttctt gtaaccaatg | 4740 |
| aaattatatt ctggctcgat tgagacagaa aaatgtggca agacatattc tttaacttca | 4800 |
| aaatatgcgg ttccagttgt tgaaaagtcc tctttatatt tagccttgat cgtccacata | 4860 |
| ccatatctag gattagacgg aatcttgaag tcaggaaaag agataattcc aatatgatca | 4920 |
| atttcttcta ccatgtcaac ttctgatcct tcaggatcta tgaaagttaa gacagtttct | 4980 |
| cttttggctg gcttcaagtc gtcattcaac gaataaactc taacttttac tgactggtct | 5040 |
| ggagtataaa caggtttgtc tgtatgaatg aagagaaatc cattgtcata ggttattggc | 5100 |
| attctttttg attttgaaaa atgctttgat acaacttcca aatacacata agaaactggg | 5160 |
| ttttgtcctc caggcaattg ttttggttgt attgttaaga ttgcagagtt ttggaattta | 5220 |
| ttctctgagg ataaatgaac atggcctgag gagtaactaa attttttatc aggataactt | 5280 |
| ttaatagaga ttgttgcatc aaatgcttca gtgtatccat aaacttgaat cacaatattt | 5340 |
| tcagatgctc caacacggaa tatttttggt gctgaaatga catatgtttg ctcctgtccc | 5400 |
| caggttttcc ccaggaagat taaaaaacaa agtattccca aaaggcccat ggttggaggt | 5460 |
| agcaggaaac cacggatata | 5480 |

In an embodiment of the disclosure the C5 iRNA (e.g., dsRNA) is characterized b the structure:

(SEQ ID NO: 363)
(3′-5′) A=A=G-C-A-A-G-A-U-
A-U-U-U-U-U-A-U-A-A-U-A-R1
(SEQ ID NO: 362)
(5′-3′) Z-Z-U=U=U-U-C-G-U-U-
C-U-A-U-A-A-A-A-A-U-A-U-U=A=U

wherein,
X is 2′-deoxy-2′-fluoro
X is 2′-O-methyl
Z is thymidine
- is

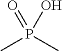

= is

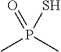

R1- is

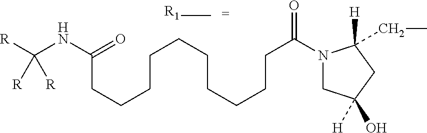

R— is

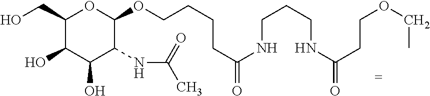 [SMILES: CCOCCC(=O)NCCCNC(=O)CCCCO[C@@H]1O[C@H](CO)[C@H](O)[C@H](O)[C@H]1NC(C)=O]

See International Nonproprietary Names for Pharmaceutical Substances (INN) (Proposed INN: List 114), WHO Drug Information, Vol. 29, No. 4, 2015.

The present disclosure includes an iRNA which can be included in a co-formulation of the present disclosure that is a double-stranded ribonucleic acid (dsRNA) agent (e.g., having a complementarity region of 19-23 nucleotides in length and/or having a strand length of no more than 30 nucleotides) for inhibiting expression of complement component C5, wherein the dsRNA agent comprises a sense strand and an antisense strand, the antisense strand comprising a region of complementarity which comprises at least 17 contiguous nucleotides differing by no more than 3 nucleotides from the nucleotide sequence of 5′-UAUUAUAAAAAUAUCUUGCUUUU-3′ (SEQ ID NO: 364), wherein one or more of the dsRNA nucleotides are modified. The dsRNA agent may include at least one modified nucleotide e.g., with 2′-deoxy, 2′-methoxy, and/or 2′-fluoro groups, for example, where substantially all of the nucleotides of the sense strand and antisense strand are modified nucleotides. Moreover, the sense strand can be conjugated to a ligand attached at the 3′-terminus, e.g., terminally modified with a triantennary GalNAc moiety.

The modified nucleotides that may be included in a dsRNA include a 3′-terminal deoxy-thymine (dT) nucleotide, a 2′-O-methyl modified nucleotide, a 2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, a nucleotide comprising a 5′-phosphorothioate group, and a terminal nucleotide linked to a cholesteryl derivative or a dodecanoic acid bisdecylamide group. The dsRNA may include a phosphorothioate and/or methylphosphonate internucleotide linkage.

A dsRNA is double stranded, but may include one or more overhangs, such as at the 3′ end of one or more strands (e.g., 2 or more nucleotides of overhang).

Double stranded RNAs of the present disclosure may include a ligand (e.g., a N-acetylgalactosamine (GalNAc) derivative,

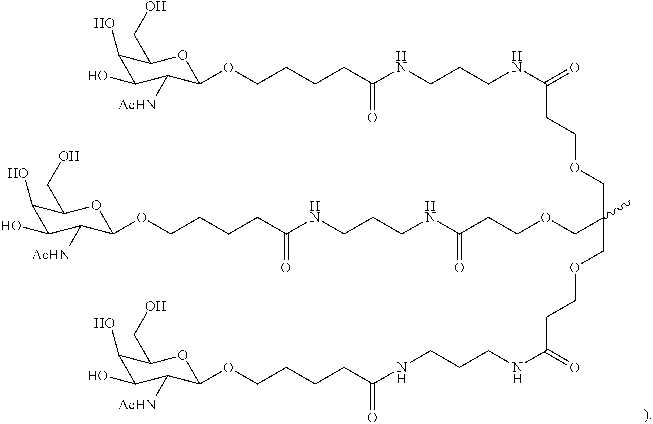 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In an embodiment of the disclosure, the ligand is conjugated to the 3′ end of the sense strand of the dsRNA.

In one aspect, the present disclosure provides a double-stranded ribonucleic acid (dsRNA) agent for inhibiting expression of complement component C5 which can be included in a co-formulation of the present disclosure, wherein the dsRNA agent comprises a sense strand and an antisense strand, wherein the sense strand comprises the nucleotide sequence 5′-AAGCAAGAUAUUUUUAUAAUA-3′ (SEQ ID NO: 365) and wherein the antisense strand comprises the nucleotide sequence 5′-UAUUAUAAAAAUAUCUUGCUUUU-3′ (SEQ ID NO: 364), e.g., wherein one or more of the dsRNA nucleotides are modified; e.g., with 2′-deoxy, 2′-methoxy, and/or 2′-fluoro groups and/or terminally modified with a triantennary GalNAc moiety. In one embodiment, the dsRNA agent comprises at least one modified nucleotide, as described herein.

In one aspect, the present disclosure provides a double stranded iRNA agent, which can be included in a co-formulation of the present disclosure, for inhibiting expression of complement component C5 wherein the double stranded iRNA agent comprises a sense strand and an antisense strand forming a double-stranded region, wherein the sense strand comprises at least 15 contiguous nucleotides differing by no more than 3 nucleotides from the nucleotide sequence of SEQ ID NO: 365 and the antisense strand comprises at least 15 contiguous nucleotides differing by no more than 3 nucleotides from the nucleotide sequence of SEQ ID NO: 364, wherein substantially all of the nucleotides of the sense strand and substantially all of the nucleotides of the antisense strand are modified nucleotides, and wherein the sense strand is conjugated to a ligand attached at the 3′-terminus. In one embodiment, the dsRNA agent comprises at least one modified nucleotide, as described herein.

In one embodiment, substantially all of the nucleotides of the sense strand are modified nucleotides selected from the group consisting of a 2′-O-methyl modification, a 2′-fluoro modification and a 3′-terminal deoxy-thymine (dT) nucleotide. In another embodiment, substantially all of the nucleotides of the antisense strand are modified nucleotides selected from the group consisting of a 2′-O-methyl modification, a 2′-fluoro modification and a 3′-terminal deoxy-thymine (dT) nucleotide. In another embodiment, the modified nucleotides are a short sequence of deoxy-thymine (dT) nucleotides. In another embodiment, the sense strand comprises two phosphorothioate intemucleotide linkages at the 5′-terminus. In one embodiment, the antisense strand comprises two phosphorothioate intemucleotide linkages at the 5′-terminus and two phosphorothioate intemucleotide linkages at the 3′-terminus. In yet another embodiment, the sense strand is conjugated to one or more GalNAc derivatives attached through a branched bivalent or trivalent linker at the 3′-terminus.

In one embodiment, at least one of the modified nucleotides is selected from the group consisting of a 3-terminal deoxy-thymine (dT) nucleotide, a 2′-O-methyl modified nucleotide, a 2′-fluoro modified nucleotide, a 2′-deoxy-modified nucleotide, a locked nucleotide, a basic nucleotide, a 2′-amino-modified nucleotide, a 2′-alkyl-modified nucleotide, a morpholino nucleotide, a phosphoramidate, a non-natural base comprising nucleotide, a nucleotide 20 comprising a 5′-phosphorothioate group, and a terminal nucleotide linked to a cholesteryl derivative or a dodecanoic acid bisdecylamide group.

In another embodiment, the modified nucleotides comprise a short sequence of 3-terminal deoxy-thymine (dT) nucleotides.

In one embodiment, the region of complementarity is at least 17 nucleotides in length. In another embodiment, the region of complementarity is between 19 and 21 nucleotides in length. In one embodiment, the region of complementarity is 19 nucleotides in length. In one embodiment, each strand is no more than 30 nucleotides in length. In one embodiment, at least one strand comprises a 3′ overhang of at least 1 nucleotide. In another embodiment, at least one strand comprises a 3′ overhang of at least 2 nucleotides. In one embodiment, the dsRNA agent further comprises a ligand. In one embodiment, the ligand is conjugated to the 3′ end of the sense strand of the dsRNA agent. In one embodiment, the ligand is an N-acetylgalactosamine (GalNAc) derivative. In one embodiment, the ligand is

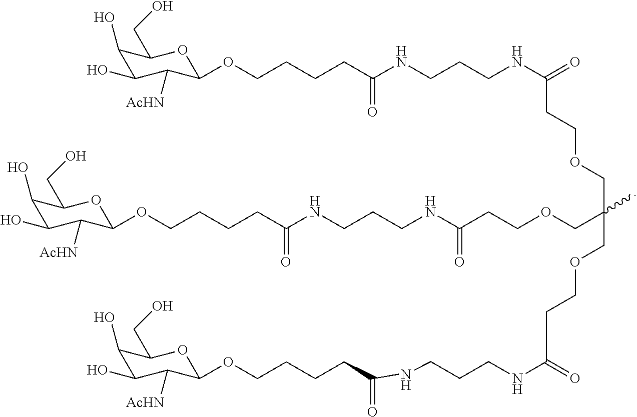

In one embodiment, the dsRNA agent is conjugated to the ligand as shown in the following schematic

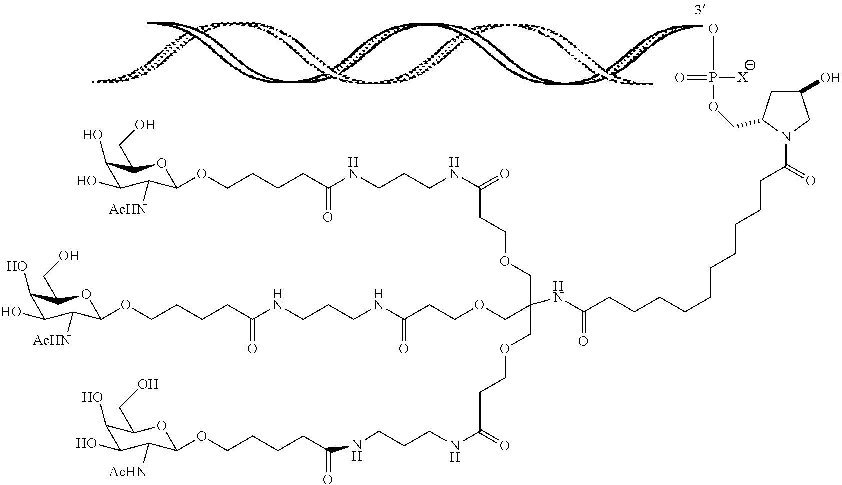

and, wherein X is O or S. In one embodiment, the X is O.

In an embodiment of the present disclosure, the C5 iRNA includes an RNA strand that is complementary to an mRNA transcribed from the C5 gene sense strand DNA sequence AAGCAAGATATTTTTATAATA, for example, wherein the iRNA is a dsRNA that includes another hybridized RNA strand.

In another aspect, the present disclosure provides a double-stranded ribonucleic acid (dsRNA) agent for inhibiting expression of complement component C5, wherein the dsRNA agent comprises a sense strand and an antisense strand, wherein the sense strand comprises the nucleotide sequence 5′-AAGCAAGAUAUUUUUAUAAUA-3′ (SEQ ID NO: 366) and wherein the antisense strand comprises the nucleotide sequence 5′-UAUUAUAAAAAUAUCUUGCUUUUdTdT-3′ (SEQ ID NO: 367).

In another aspect, the present disclosure provides a double-stranded ribonucleic acid (dsRNA) agent for inhibiting expression of complement component C5, wherein the dsRNA agent comprises a sense strand and an antisense strand, wherein the sense strand comprises the nucleotide sequence asasGfcAfaGfaUfAfUfuUfuuAfuAfauaL96 (SEQ ID NO: 368) and wherein the antisense strand comprises the nucleotide sequence usAfsUfuAfuaAfaAfauaUfcUfuGfcuususudTdT (SEQ ID NO: 369).

In an embodiment of the disclosure, the sense or antisense strands of a dsRNA that can be included in a formulation of the present disclosure comprises sequences selected from the group consisting of A-118320, A-118321, A-118316, A-118317, A-118332, A-118333, A-118396, A-118397, A-118386, A-118387, A-118312, A-118313, A-118324, A-118325, A-119324, A-119325, A-119332, A-119333, A-119328, A-119329, A-119322, A-119323, A-119324, A-119325, A-119334, A-119335, A-119330, A-119331, A-119326, A-I119327, A-125167, A-125173, A-125647, A-125157, A-125173, and A-125127. In one embodiment, the dsRNA agent comprises at least one modified nucleotide.

TABLE C
Sense and Anti-sense RNA strands of C5 dsRNAs
(unmodified and modified strands shown)

| | |
|---|---|
| A-118320 | aagcaagaua uuuuuauaau a |
| (SEQ ID NO: 370) | AfaGfcAfaGfaUfAfUfuUfuUfaUfaAfuAfL96 |
| sense strand | |
| A-118321 | uauuauaaaa auaucuugcu uuu |
| anti-sense strand | uAfuUfaUfaAfaAfauaUfcUfuGfcUfusUfsu |
| (SEQ ID NO: 371) | |
| A-118316 | gacaaaauaa cucacuauaa u |
| sense strand | GfaCfaAfaAfuAfAfCfuCfaCfuAfuAfaUfL96 |
| (SEQ ID NO: 372) | |
| A-118317 | auuauaguga guuauuuugu caa |
| anti-sense strand | aUfuAfuAfgUfgAfguuAfuUfuUfgUfcsAfsa |
| (SEQ ID NO: 373) | |
| A-118332 | aggauuuuga guguaaaagg a |
| sense strand | AfgGfaUfuUfuGfAfGfuGfuAfaAfaGfgAfL96 |
| (SEQ ID NO: 374) | |
| A-118333 | uccuuuuaca cucaaaaucc uuu |
| anti-sense strand | |
| (SEQ ID NO: 375) | |
| A-118396 | guuccggaua uuugaacuuu u |
| sense strand | GfuUfcCfgGfaUfAfUfuUfgAfaCfuUfuUfL96 |
| (SEQ ID NO: 376) | |
| A-118397 | aaaaguucaa auauccggaa ccg |
| anti-sense strand | aAfaAfgUfuCfaAfauaUfcCfgGfaAfcsCfsg |
| (SEQ ID NO: 377) | |
| A-118386 | cagaucaaac acaauuucag u |
| sense strand | CfaGfaUfcAfaAfCfAfcAfaUfuUfcAfgUfL96 |
| (SEQ ID NO: 378) | |
| A-118387 | acugaaauug uguuugaucu gca |
| anti-sense strand | aCfuGfaAfaUfuGfuguUfuGfaUfcUfgsCfsa |
| (SEQ ID NO: 379) | |
| A-118312 | ugacaaaaua acucacuaua a |
| sense strand | UfgAfcAfaAfaUfAfAfcUfcAfcUfaUfaAfL96 |
| (SEQ ID NO: 380) | |
| A-118313 | uuauagugag uuauuuuguc aau |
| anti-sense strand | uUfaUfaGfuGfaGfuuaUfuUfuGfuCfasAfsu |
| (SEQ ID NO: 381) | |
| A-118324 | auuuaaacaa caaguaccuu u |
| sense strand | AfuUfuAfaAfcAfAfCfaAfgUfaCfcUfuUfL96 |
| (SEQ ID NO: 382) | |
| A-118325 | aaagguacuu guuguuuaaa ucu |
| anti-sense strand | AfuUfuAfaAfcAfAfCfaAfgUfaCfcUfuUfL96 |
| (SEQ ID NO: 383) | |
| A-119324 | gacaaaauaa cucacuauaa u |
| sense strand | AfuUfuAfaAfcAfAfCfaAfgUfaCfcUfuUfL96 |
| (SEQ ID NO: 384) | |
| A-119325 | auuauaguga guuauuuugu caa |
| anti-sense strand | asUfsuAfuAfgUfgAfguuAfuUfuUfgUfcsasa |
| (SEQ ID NO: 385) | |
| A-119332 | cagaucaaac acaauuucag u |
| sense strand | CfsasGfaUfcAfaAfCfAfcAfaUfuUfcAfgUfL96 |
| (SEQ ID NO: 386) | |
| A-119333 | acugaaauug uguuugaucu gca |
| anti-sense strand | asCfsuGfaAfaUfuGfuguUfuGfaUfcUfgscsa |
| (SEQ ID NO: 387) | |
| A-119328 | auuuaaacaa caaguaccuu u |
| sense strand | AfsusUfuAfaAfcAfAfCfaAfgUfaCfcUfuUfL96 |
| (SEQ ID NO: 388) | |
| A-119329 | aaagguacuu guuguuuaaa ucu |
| anti-sense strand | asAfsaGfgUfaCfuUfguuGfuUfuAfaAfusesu |
| (SEQ ID NO: 389) | |
| A-119322 | ugacaaaaua acucacuaua a |
| sense strand | UfsgsAfcAfaAfaUfAfAfcUfcAfcUfaUfaAfL96 |
| (SEQ ID NO: 390) | |
| A-119323 | uuauagugag uuauuuuguc aau |
| anti-sense strand | |
| (SEQ ID NO: 391) | |
| A-119324 | gacaaaauaa cucacuauaa u |
| sense strand | GfsasCfaAfaAfuAfAfCfuCfaCfuAfuAfaUfL96 |
| (SEQ ID NO: 392) | |
| A-119325 | auuauagugaguuauuuugucaa |
| anti-sense strand | asUfsuAfuAfgUfgAfguuAfuUfuUfgUfcsasa |
| (SEQ ID NO: 393) | |
| A-119334 | guuccggaua uuugaacuuu u |
| sense strand | GfsusUfcCfgGfaUfAfUfuUfgAfaCfuUfuUfL96 |
| (SEQ ID NO: 394) | |
| A-119335 | aaaaguucaa auauccggaa ccg |
| anti-sense strand | asAfsaAfgUfuCfaAfauaUfcCfgGfaAfesesg |
| (SEQ ID NO: 395) | |
| A-119330 | ugcagaucaa acacaauuuc a |
| sense strand | UfsgsCfaGfaUfcAfAfAfcAfcAfaUfuUfcAfL96 |
| (SEQ ID NO: 396) | |
| A-119331 | ugaaauugug uuugaucugc aga |
| anti-sense strand | usGfsaAfaUfuGfuGfuuuGfaUfcUfgCfasgsa |
| (SEQ ID NO: 397) | |
| A-119326 | aagcaagaua uuuuuauaau a |
| sense strand | AfsasGfcAfaGfaUfAfUfuUfuUfaUfaAfuAfL96 |
| (SEQ ID NO: 398) | |
| A-119327 | uauuauaaaa auaucuugcu uuu |
| | usAfsuUfaUfaAfaAfauaUfcUfuGfcUfususu |
| anti-sense strand | |
| (SEQ ID NO: 399) | |
| A-125167 | aagcaagaua uuuuuauaau a |
| sense strand | asasGfcAfaGfaUfAfUfuUfuuAfuAfauaL96 |
| (SEQ ID NO: 400) | |
| A-125173 | uauuauaaaa auaucuugcu uuutt |
| anti-sense strand | usAfsUfuAfuAfAfaAfauaUfcUfuGfcuususudTdT |
| (SEQ ID NO: 401) | |
| A-125647 | uauuauaaaa auaucuugcu uuutt |
| anti-sense stand | usAfsUfuAfuaAfaAfauaUfcUfuGfcuususudTdT |
| (SEQ ID NO: 402) | |
| A-125157 | aagcaagaua uuuuuauaau a |
| sense strand | asasGfcAfaGfaUfAfUfuUfuuAfuaAfuaL96 |
| (SEQ ID NO: 403) | |
| A-125173 | uauuauaaaa auaucuugcu uuutt |
| anti-sense strand | usAfsUfuAfuAfAfaAfauaUfcUfuGfcuususudTdT |
| (SEQ ID NO: 404) | |
| A-125127 | aagcaagaua uuuuuauaau a |
| sense strand | asasgcaagaUfaUfuuuuauaauaL96 |
| (SEQ ID NO: 405) | |

wherein,
A=Adenosi ne-3′-phosphate
Af=2′-fluoroadenosine-3′-phosphate
Afs=2′-fluoroadenosine-3′-phosphorothioate
As=adenosine-3′-phosphorothioate
C=cytidine-3′-phosphate
Cf=2′-fluorocytidine-3′-phosphate
Cfs=2′-fluorocytidine-3′-phosphorothioate
Cs=cytidine-3′-phosphorothioate
G=guanosine-3′-phosphate
Gf=2′-fluoroguanosine-3′-phosphate
Gfs=2′-fluoroguanosine-3′-phosphorothioate
Gs=guanosine-3′-phosphorothioate
T=5′-methyluridine-3-phosphate
Tf=2′-fluoro-5-methyluridine-3′-phosphate
Tfs=2′-fluoro-5-methyluridine-3′-phosphorothioate
Ts=5-methyluridine-3-phosphorothioate
U=Uridine-3′-phosphate
Uf=2′-fluorouridine-3-phosphate
Ufs=2′-fluorouridine-3-phosphorothioate
Us=uridine-3′-phosphorothioate
N=any nucleotide (G, A, C, T or U)
a=2′-O-methyladenosine-3′-phosphate
as=2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
t=2′-O-methyl-5-methyluridine-3-phosphate
ts=2′-O-methyl-5-methyluridine-3-phosphorothioate
u=2′-O-methyluridine-3′-phosphate
us=2′-O-methyluridine-3′-phosphorothioate
s=phosphorothioate linkage
L96=N-[tris(GalNAc-alkyl)-amidodecanoyl)]-4-hydroxyprolinol
Hyp—(GalNAc-alkyl)₃
(dt)=deoxy-thymine

In an embodiment of the disclosure, the dsRNA comprises the two following pairs of strands:
A-118320 & A-118321
A-118316 & A-118317
A-118332 & A-118333
A-118396 & A-118397
A-118386 & A-118387
A-118312 & A-118313
A-118324 & A-118325
A-119324 & A-119325
A-119332 & A-119333
A-119328 & A-119329
A-119322 & A-119323
A-119324 & A-119325
A-119334 & A-119335
A-119330 & A-119331
A-119326 & A-119327
A-125167 & A-125173 or A-125647
A-125157 & A-125173 or A-125647
A-125127 & A-125173 or A-125647

In an embodiment of the disclosure, the C5 iRNA (e.g., Cemdisiran) comprises one or more alactosamines e.g., 3, for example, represented by the structure:

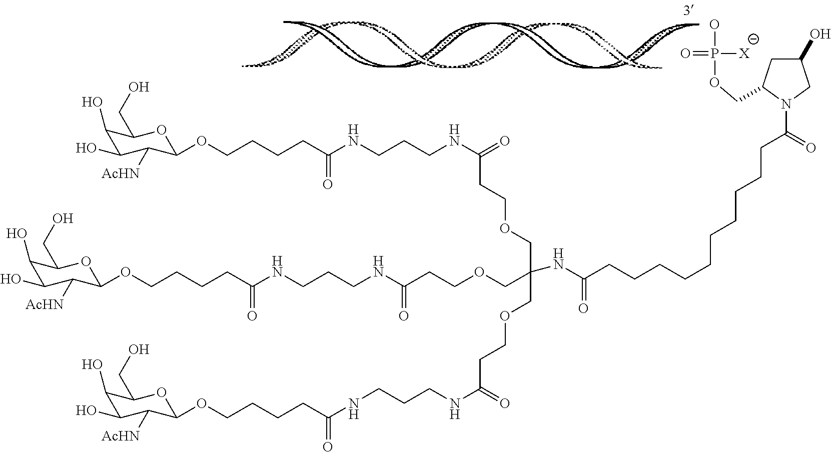

wherein the wavy double helix-like structure represents the RNA portion of the molecule and X is O or X is S; e.g.,

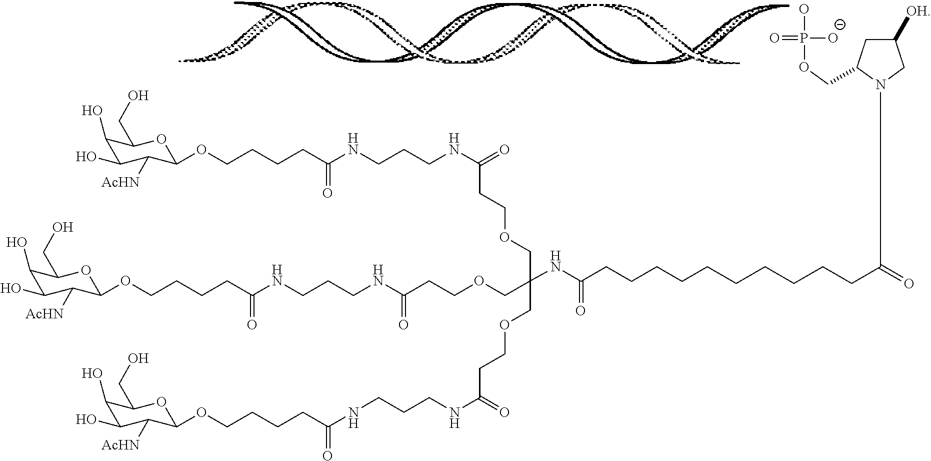 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2COP(=O)([O-])O)OC(CO)C(O)C1O]

However, in an embodiment of the disclosure, a co-formulation of the present disclosure further comprises degradation products represented by one or more of the following structures (wavy line represents double stranded RNA structure):

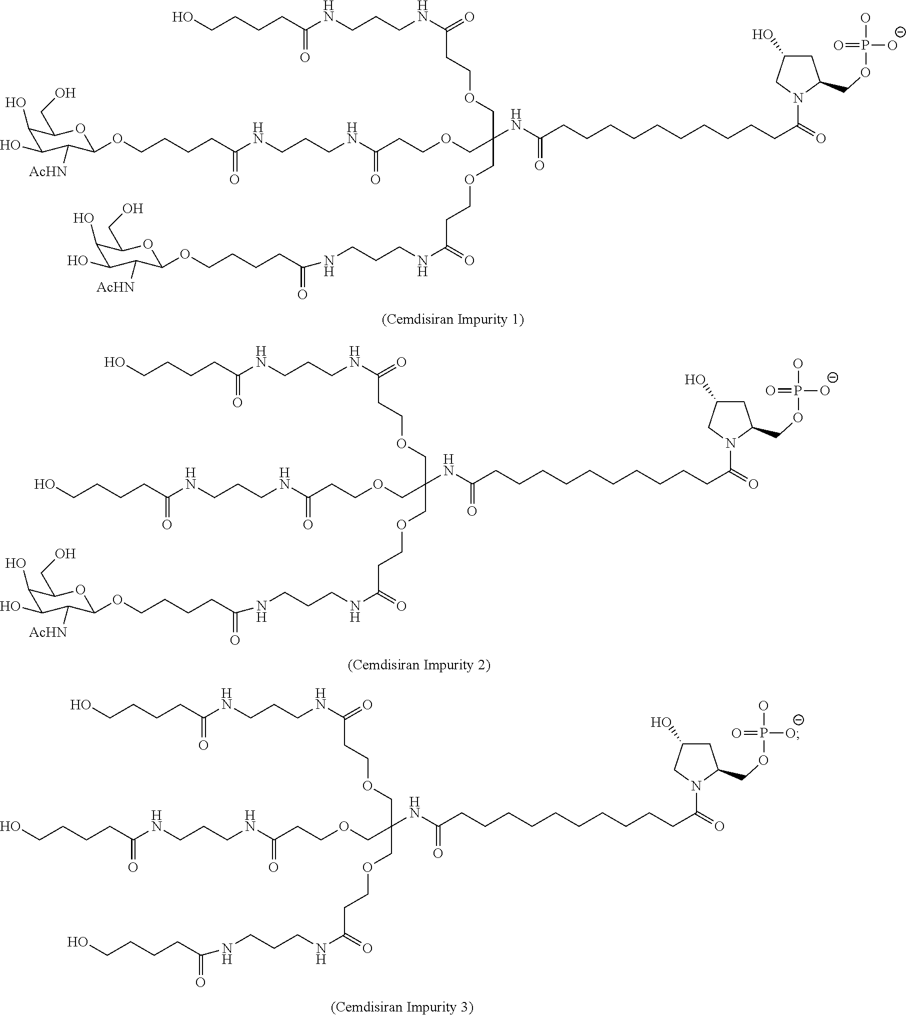

wherein 1, 2 or 3 of the terminal N-acetylgalactosamines (GalNAc) are missing.

iRNAs of the present disclosure can be chemically linked, by the RNA portion of the molecule, to one or more ligands, moieties or conjugates that enhance the activity, cellular distribution or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

A ligand can be a carbohydrate. A carbohydrate conjugated RNA is advantageous for the in vivo delivery of nucleic acids. As used herein, “carbohydrate” ligand refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate for use in the compositions and methods of the disclosure is a monosaccharide. In one embodiment, the monosaccharide is an N-acetylgalactosamine, such as

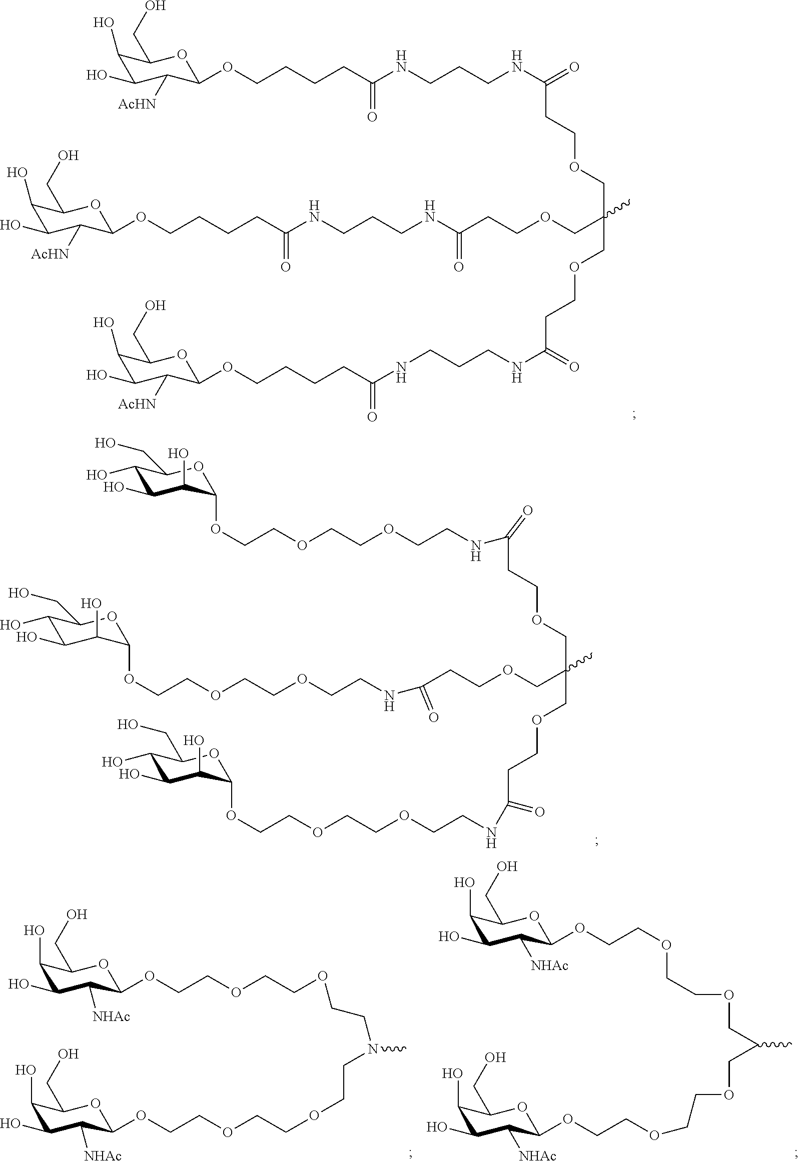

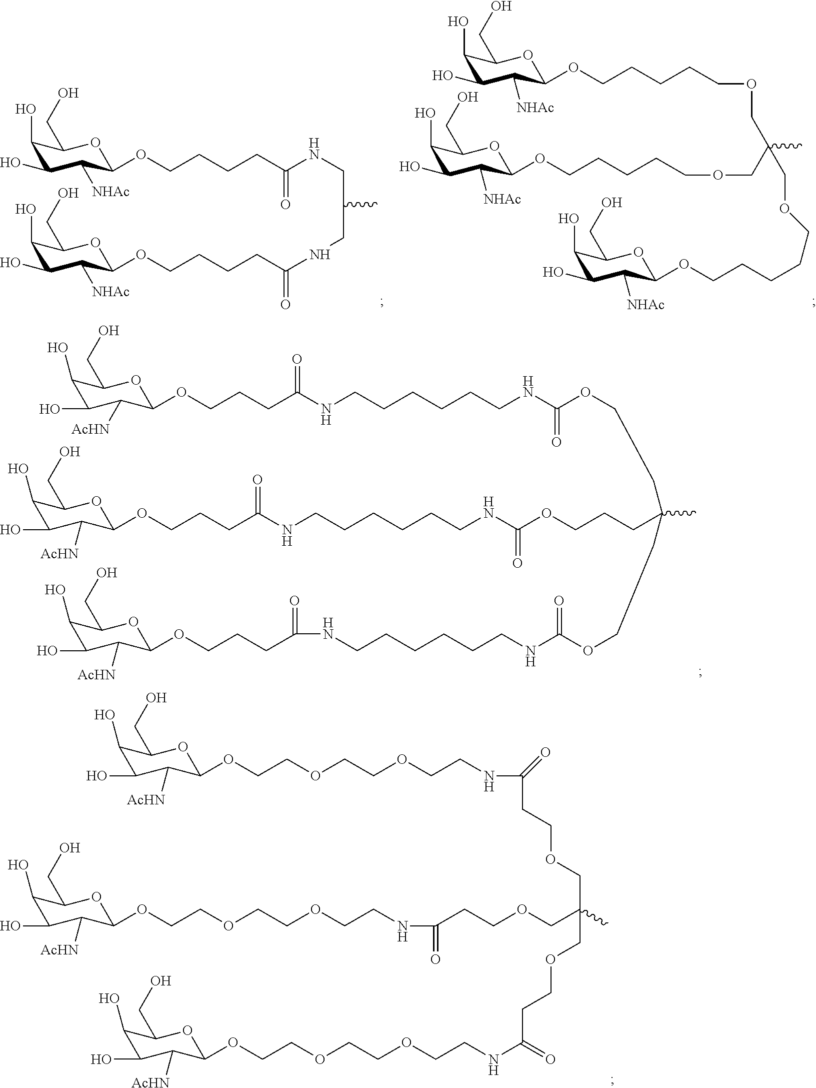

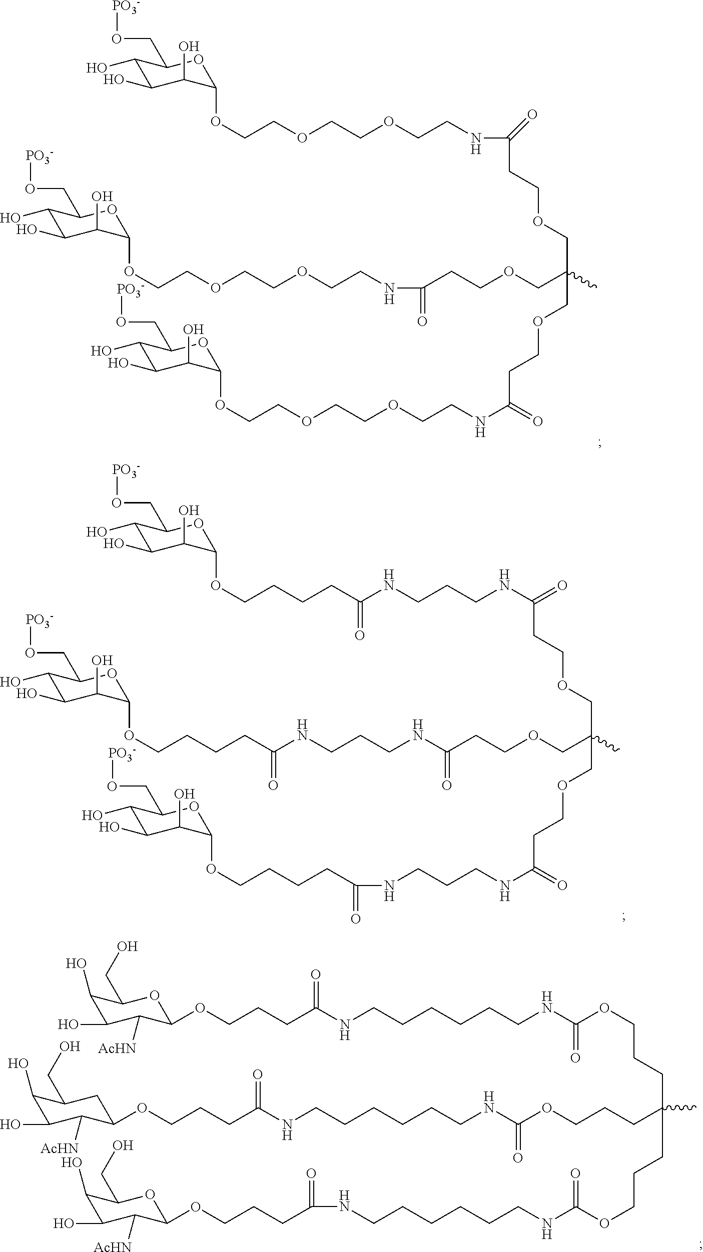

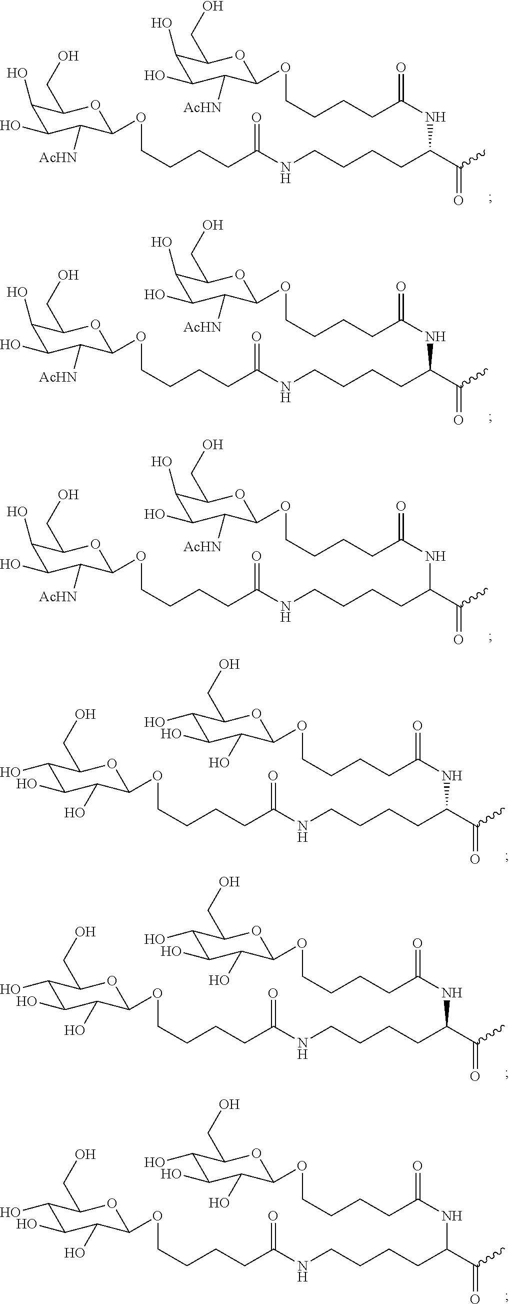

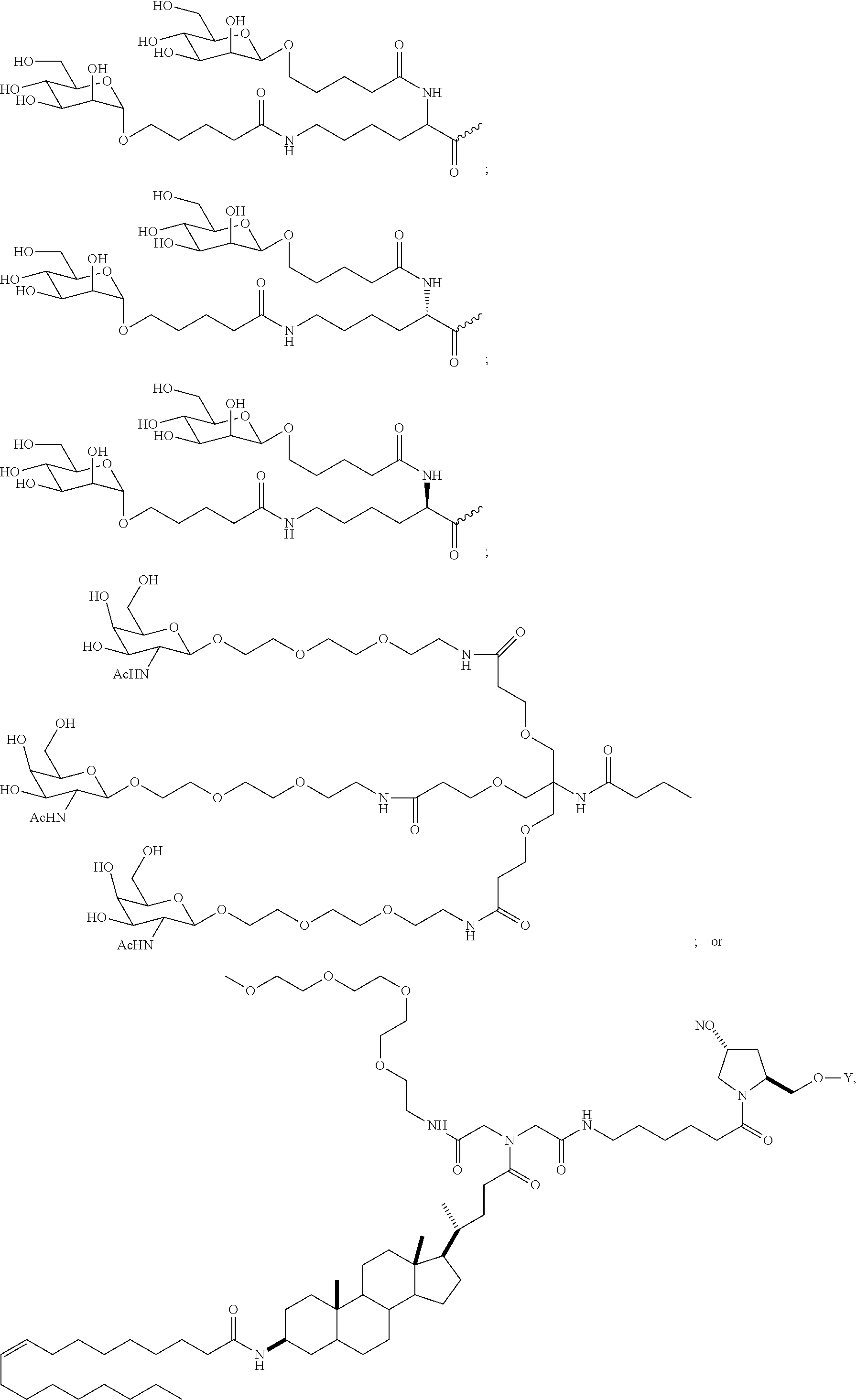

when one of X or Y is an oligonucleotide, the other is a hydrogen.

In some embodiments, the conjugate or ligand described herein can be attached to an iRNA oligonucleotide with various linkers that can be cleavable or non-cleavable. The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-16, or 8-16 atoms. Linkers may comprise redox cleavable linking groups, phosphate-based cleavable linking groups, acid cleavable linking groups, ester-based linking groups and/or peptide-based cleaving groups.

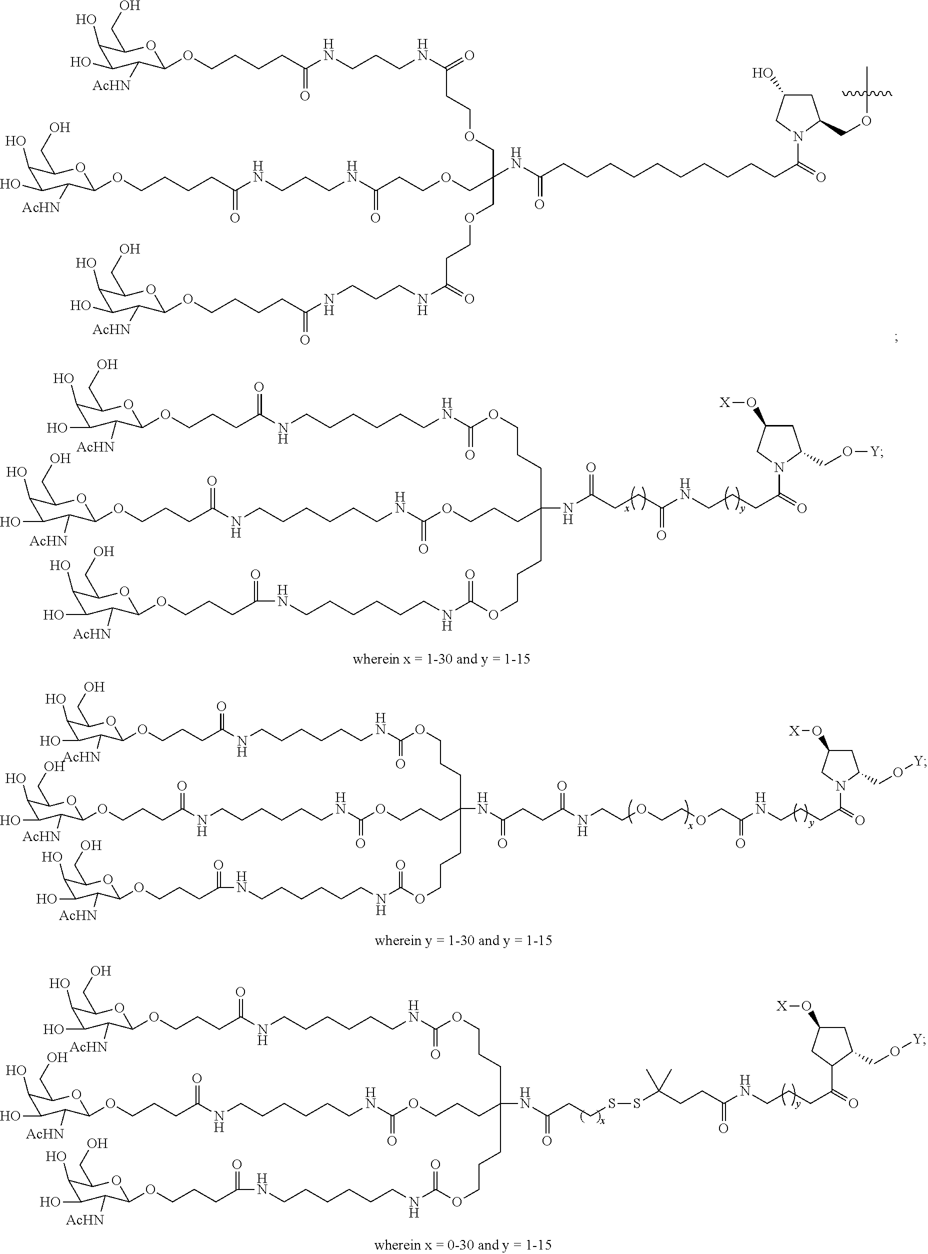

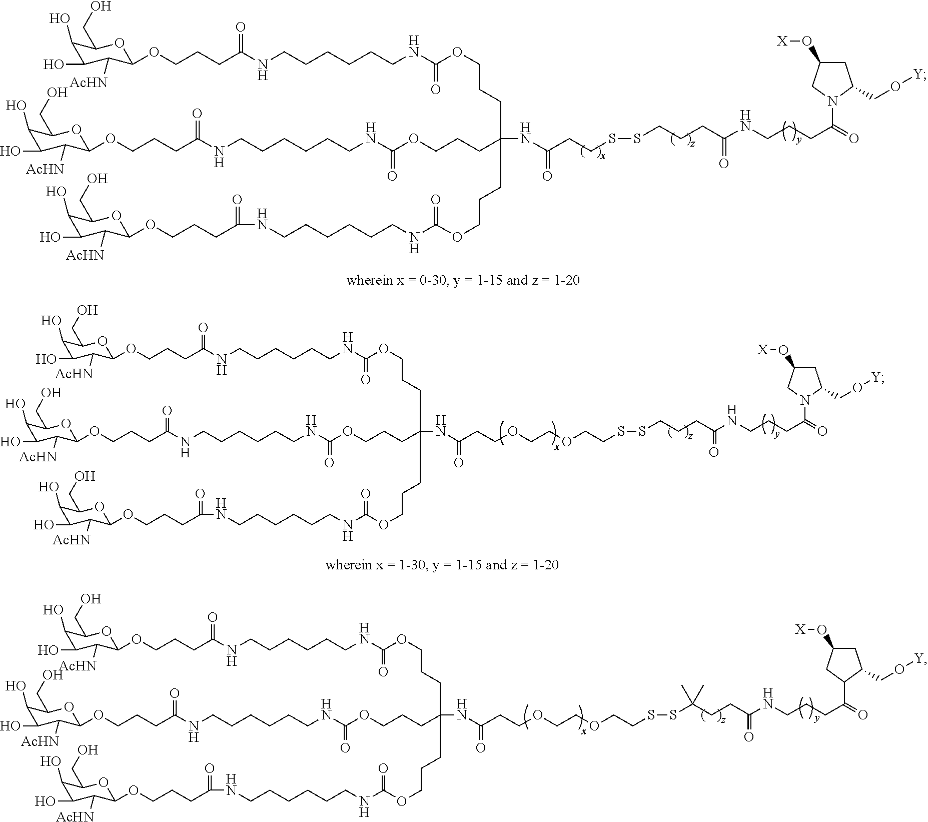

wherein x=1-30, y=1-15 and z=1-20; when one of X or Y is an oligonucleotide, the other is a hydrogen. In certain embodiments of the compositions and methods of the disclosure, a ligand is one or more GalNAc (N-acetylgalactosamine) derivatives attached through a bivalent or trivalent branched linker.

### Co-Formulations

The present disclosure provides pharmaceutical, preferably aqueous, co-formulations that comprise a pharmaceutically acceptable carrier and the separate components (i) an anti-C5 antibody or antigen-binding fragment thereof (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H-4H-12171P; H4H12175P; H4H12176P2; H4H12177P2; H4H12183P2; H2M11682N; H2M11684N; H2M11694N; H2M11695N; crovalimab; Eculizumab, tesidolumab, mubodina or Ravulizumab; preferably, Pozelimab) and; (ii) a C5 iRNA, preferably a glycoconjugate, such as Cemdisiran.

A co-formulation may be designated in the form: antibody/iRNA; for example, “Pozelimab/Cemdisiran” or “Cemdisiran/Pozelimab” denotes a co-formulation of the present disclosure including Pozelimab and Cemdisiran.

A co-formulation or pharmaceutical co-formulation, as used herein, refers to a formulation including an anti-C5 antigen-binding protein (e.g., antibody or antigen-binding fragment thereof), a C5 iRNA and a pharmaceutically acceptable carrier. A pharmaceutically acceptable carrier includes, for example, one or more excipients. In an embodiment of the disclosure, a co-formulation of the present disclosure is aqueous, i.e., includes water.

Pharmaceutical formulations including anti-C5 antigen-binding proteins may be prepared by admixing the antigen-binding protein with one or more excipients (see, e.g., Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis et al. (eds.) (1993) Pharmaceutical Dosage Forms: Parenteral Medications, Marcel Dekker, NY; Lieberman et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

The present invention provides a method for making a co-formulation comprising combining a C5 iRNA (e.g., Cemdisiran or the Na⁺ salt thereof; e.g., wherein the C5 iRNA is reconstituted with water from a lyophilized composition thereof); an antibody or antigen-binding fragment thereof that binds specifically to C5 (e.g., Pozelimab); a buffer (e.g., Histidine); a viscosity reducer (e.g., L-arginine); a stabilizer (e.g., sucrose); and a non-ionic surfactant (e.g., polysorbate 80), and, optionally, adjusting the co-formulation pH to greater than or less than about 6 (e.g., about 6.5±0.2); and, optionally sterile filtering the co-formulation.

The present disclosure provides methods for making a co-formulation of the present disclosure including combining an RNAi (e.g., Cemdisiran) and the antibody or antigen-binding fragment (e.g., Pozelimab) (e.g., that includes detectable quantities of beta-hexosaminidase contaminant), and (i) adding GalNAc to the co-formulation and/or (ii) adjusting the pH of the co-formulation to about or below about 6 (e.g., within not less than 0.5). In an embodiment of the disclosure, other excipients are also combined, e.g., buffer, viscosity reducer, stabilizer and/or surfactant. Co-formulations (e.g., Cemdisiran/Pozelimab) produced by such methods are part of the present disclosure. In an embodiment of the disclosure, the antibody or fragment which is combined with the other components is initially in a lot that includes beta-hexosaminidase contaminant and is diluted by a factor of 0.25, 0.5 or 0.75 when incorporated into the co-formulation.

Various viscosity reducer are known in the art for use with co-formulations. viscosity reducer are agents that can reduce the viscosity of a formulation. Viscosity reducers may also function as tonicifiers that modulate the osmolality of the formulation. Such viscosity reducer include an adipic acid; an amino acid or salt thereof; (D- or L-) arginine; L-arginine HCl; (D- or L-) alanine; benzenesulfonic acid; caffeine; a dicarboxylic acid; an ester of citric acid; (D- or L-) glutamate; Glycine; (D- or L-) histidine; an inorganic salt; L-Ornithine; (D- or L-) lysine; Proline; (D- or L-) phenylalanine; (D- or L-) serine; NaCl; pyridoxamine; pyridoxine; thiamine phosphoric acid ester chloride dihydrate; triethyl citrate; (D- or L-) valine; and/or a xanthine. In an embodiment of the disclosure, the amino acid is an L-amino acid such as L-arginine. L-arginine acts both as a tonicifier as well as a stabilizer and viscosity reducer. Arginine HCl can decrease Cemdisiran degradation and allow for a near isotonic solution.

Stabilizers include agents, such as sugars or polyols, that aid in the reduction of degradation, for example, of antibodies or antigen-binding fragments, e.g., aggregation. Polyols are sugar alcohols having multiple hydroxyl groups. Stabilizers include a sugar or polyol, e.g., trehalose, sorbitol, mannitol, taurine, propane sulfonic acid, L-proline, sucrose, glycerol, threitol, maltitol, and/or polyethylene glycol (PEG; such as PEG3350).

Non-ionic surfactants contain molecules with head groups that are uncharged. Non-ionic surfactants include a non-ionic surfactant including a polyoxyethylene moiety; a sorbitan; a polyoxyethylene glycol alkyl ether, such as octaethylene glycol monododecyl ether; pentaethylene glycol monododecyl ether; polyoxypropylene glycol alkyl ether; glucoside alkyl ether, such as decyl glucoside, lauryl glucoside, octyl glucoside; polyoxyethylene glycol octylphenol ether, such as triton X-100; polyoxyethylene glycol alkylphenol ether, such as nonoxynol-9; glycerol alkyl ester, such as glyceryl laurate; polyoxyethylene glycol sorbitan alkyl ester, such as polysorbate; sorbitan alkyl ester, such as spans; cocamide MEA, cocamide DEA, dodecyldimethylamine oxide; block copolymer of polyethylene glycol and polypropylene glycol, such as poloxamer; and polyethoxylated tallow amine (POEA); poloxamer 188, polyethylene glycol 3350, a polyethylene glycol (e.g., PEG3350) or a polysorbate such as polysorbate 80 (PS80) or polysorbate 20 (PS20). In an embodiment of the disclosure, the non-ionic detergent is polysorbate-20 (PS20), polysorbate-80 (PS80).

A buffer is a mixture of a weak acid and its conjugate base or vice versa which resists changes in its pH and therefore keeps the pH at a nearly constant value. Various buffers may be used in the co-formulations of the present disclosure, for example, histidine-based buffer, phosphate buffer or citrate buffer. A histidine-based buffer is a buffer comprising histidine. Examples of histidine buffers include histidine chloride, histidine hydrochloride, histidine acetate, histidine phosphate, and histidine sulphate.

The present disclosure encompasses co-formulations having any of the specifically recited components, e.g., at the specifically recited concentrations, but wherein the pH of the co-formulation is about 6.5.

In an embodiment of the disclosure, the co-formulation contains the impurity, beta-hexosaminidase, e.g., in a quantity of about 0.04 to about 0.17 micrograms/ml, e.g., when pH of the co-formulation is less than or greater than about 6 (e.g., by at least 0.5), e.g., 6.5.

For example, the disclosure includes a pharmaceutical co-formulation (e.g., Cemdisiran/Pozelimab) comprising:
One or more C5 iRNAs, for example as set forth herein (e.g., Cemdisiran, preferably the Na⁺ form), for example, at a concentration of about 20-100, 20, 25, 30, 35, 40, 45, 50, 55, 60, 65, 70, 75, 80, 85, 90, 95, 100 mg/ml (or higher, e.g., 110, 115, 120, 130, 140, 150, 155, 160, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg/ml); wherein the free acid form content can be determined by multiplying the Na⁺ form concentration is multiplied by 0.9443;
One or more anti-C5 antibodies or antigen-binding fragments thereof (e.g., Pozelimab), for example, at a concentration from about 90 to about 275 mg/ml (e.g., about 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 211, 220, 242, 274 mg/ml) or at least about 150 mg/ml, at least about 175 mg/ml, at least about 200 mg/ml, at least about 211 mg/ml, at least about 220 mg/ml, at least about 242 mg/ml or at least about 274 mg/ml);
A viscosity reducer such as L-arginine (e.g., L-arginine HCl) (e.g., at a concentration from about 40-140 mM, e.g., about 50 mM or 90 mM) (e.g., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139 or 140 mM);
A stabilizer, such as a sugar or polyol (e.g., at a concentration from about 0.8 to about 3.6% (w/v), for example about 1%) (e.g., 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6% (w/v));
A non-ionic surfactant, such as polysorbate 80 (PS80) or polysorbate 20 (PS20) (e.g., at a concentration of about 0.025 to about 0.2% (w/v), e.g., about 0.075% (w/v) (e.g., 0.025, 0.05, 0.075, 0.1, 0.125, 0.15, 0.175, 0.2% (w/v)));
A buffer such as a histidine-based buffer (e.g., at a concentration from about 10 to about 50 mM, for example about 30 mM (e.g., 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50 mM)); and having
A pH of about 5.5 to about 7.0, e.g., about 6.5; or within not less than 0.5 of pH 6.0.

In an embodiment of the disclosure, a co-formulation (e.g., Cemdisiran/Pozelimab) comprises (e.g., for example, with detectable quantities of beta-hexosaminidase as discussed herein):
A double stranded C5 iRNA that is conjugated to a triantennary GalNAc moiety; and an anti-C5 antibody or antigen-binding fragment thereof that was expressed and isolated from a mammalian host cell that includes beta-hexosaminidase,
a pH above or below (by at least 0.5) 6.0;
a C5 iRNA (e.g., that is conjugated to a triantennary GalNAc moiety),
an anti-C5 antibody or antigen-binding fragment thereof (e.g., that was expressed and isolated from a mammalian host cell that includes beta-hexosaminidase),
a buffer,
a viscosity reducer,
a stabilizer, and
a non-ionic surfactant;
a C5 iRNA (e.g., that is conjugated to a triantennary GalNAc moiety),
an anti-C5 antibody or antigen-binding fragment thereof (e.g., that was expressed and isolated from a mammalian host cell that includes beta-hexosaminidase),
Histidine-based buffer,
L-arginine,
a stabilizer, and
a non-ionic surfactant;
a C5 iRNA (e.g., that is conjugated to a triantennary GalNAc moiety),
An anti-C5 antibody or antigen-binding fragment thereof (e.g., that was expressed and isolated from a mammalian host cell that includes beta-hexosaminidase),
Histidine-based buffer,
L-arginine,
a sugar or polyol, and
a non-ionic surfactant;
Cemdisiran,
Pozelimab (e.g., that was expressed and isolated from a mammalian host cell that includes beta-hexosaminidase),
Histidine-based buffer,
L-arginine,
a stabilizer, and
a non-ionic surfactant,
pH about 6.5;
Cemdisiran,
Pozelimab (e.g., that was expressed and isolated from a mammalian host cell that includes beta-hexosaminidase),
Histidine-based buffer,
L-arginine,
sucrose, and
polysorbate 80,
pH about 6.5;
100 (±10) mg/mL C5 iRNA such as Cemdisiran (Na⁺ form),
100 (±10) mg/mL anti-C5 antibody or antigen-binding fragment thereof such as Pozelimab (e.g., that was expressed and isolated from a mammalian host cell that includes beta-hexosaminidase),
50 (±10) mM viscosity reducer such as L-arginine (e.g., L-arginine HCl),
10 (±2) mM buffer such as histidine-based buffer,
1.0 (±0.2)% stabilizer such as sucrose,
0.075 (±0.00375)% non-ionic surfactant such as PS80,
pH 6.5;
75 (±7.5) mg/mL C5 iRNA such as Cemdisiran (Na⁺ form),
150 (±15) mg/mL anti-C5 antibody or antigen-binding fragment thereof such as Pozelimab (e.g., that was expressed and isolated from a mammalian host cell that includes beta-hexosaminidase),
75 (±15) mM viscosity reducer such as L-arginine (e.g., L-arginine HCl),
15 (±3) mM buffer such as histidine-based buffer,
1.5 (±0.3)% stabilizer such as sucrose,
0.1125 (±0.056)% non-ionic surfactant such as PS80,
pH 6.5;
50 (±5) mg/mL C5 iRNA such as Cemdisiran (Na⁺ form),
100 (±10) mg/mL anti-C5 antibody or antigen-binding fragment thereof such as Pozelimab (e.g., that was expressed and isolated from a mammalian host cell that includes beta-hexosaminidase),
75 mM (±15) viscosity reducer such as L-arginine (e.g., L-arginine HCl),
15 (±3) mM buffer such as histidine-based buffer,
1.5 (±0.3)% stabilizer such as sucrose,
0.1125 (±0.056)% non-ionic surfactant such as PS80;
pH 6.5;
50 (±5) mg/mL C5 iRNA such as Cemdisiran (Na⁺ form),
100 (±10) mg/mL anti-C5 antibody or antigen-binding fragment thereof such as Pozelimab (e.g., that was expressed and isolated from a mammalian host cell that includes beta-hexosaminidase),
75 (±15) mM viscosity reducer such as L-arginine (e.g., L-arginine HCl),
35 (±7) mM buffer such as histidine-based buffer,
1.5 (±0.3)% stabilizer such as sucrose,
0.1125 (±0.056)% non-ionic surfactant such as PS80,
pH 6.5;
100 (±10) mg/mL C5 iRNA such as Cemdisiran (Na⁺ form),
100 (±10) mg/mL anti-C5 antibody or antigen-binding fragment thereof such as Pozelimab (e.g., that was expressed and isolated from a mammalian host cell that includes beta-hexosaminidase),
50 (±10) mM viscosity reducer such as L-arginine (e.g., L-arginine HCl),
30 (±6) mM buffer such as histidine-based buffer,
1 (±0.2)% stabilizer such as sucrose,
0.075 (±0.00375)% non-ionic surfactant such as PS80,
pH 6.5;
50 (±5) mg/mL C5 iRNA such as Cemdisiran (Na⁺ form),
100 (±10) mg/mL anti-C5 antibody or antigen-binding fragment thereof such as Pozelimab (e.g., that was expressed and isolated from a mammalian host cell that includes beta-hexosaminidase),
90 (±18) mM viscosity reducer such as L-arginine (e.g., L-arginine HCl),
30 (±6) mM buffer such as histidine-based buffer,
1 (±0.2)% stabilizer such as sucrose,
0.075 (±0.00375)% non-ionic surfactant such as PS80,
pH 6.5;
100 mg/mL Cemdisiran (Na⁺ form),
100 mg/mL Pozelimab,
50 mM L-arginine (e.g., L-arginine HCl),
30 mM histidine-based buffer,
1% (w/v) sucrose,
0.075% (w/v) PS80,
pH 6.5;
50 mg/mL Cemdisiran (Na⁺ form),
100 mg/mL Pozelimab,
90 mM L-arginine (e.g., L-arginine HCl),
30 mM histidine-based buffer,
1% (w/v) sucrose,
0.075% (w/v) PS80,
pH 6.5;
100 mg/mL Cemdisiran (Na⁺ form),
100 mg/mL Pozelimab,
50 mM L-arginine (e.g., L-arginine HCl),
10 mM histidine-based buffer,
1.0% sucrose,
0.075% PS80,
pH 6.5;
75 mg/mL Cemdisiran (Na⁺ form),
150 mg/mL Pozelimab,
75 mM L-arginine (e.g., L-arginine HCl),
15 mM histidine-based buffer,
1.5% sucrose,
0.1125% PS80,
pH 6.5;
50 mg/mL Cemdisiran (Na⁺ form),
100 mg/mL Pozelimab,
75 mM L-arginine (e.g., L-arginine HCl),
15 mM histidine-based buffer,
1.5% sucrose,
0.1125% PS80;
pH 6.5;
50 mg/mL Cemdisiran (Na⁺ form),
100 mg/mL Pozelimab,
75 mM L-arginine (e.g., L-arginine HCl),
35 mM histidine-based buffer,
1.5% sucrose,
0.1125% PS80,
pH 6.5;
100 mg/mL Cemdisiran (Na⁺ form),
100 mg/mL Pozelimab,
50 mM L-arginine (e.g., L-arginine HCl),
30 mM histidine-based buffer,
1% sucrose,
0.075% PS80,
pH 6.5;
47.2 mg/mL Cemdisiran (Free Acid Form (FAF)) molecule, which may be any salt form, such as Na⁺,
100 mg/mL Pozelimab,
30 mM histidine,
90 mM L-arginine,
1% (w/v) sucrose,
0.075% (w/v) polysorbate 80 (e.g., super refined grade (SR)),
pH 6.5
50 mg/mL Cemdisiran (Na⁺ form),
100 mg/mL Pozelimab,
90 mM L-arginine (e.g., L-arginine HCl),
30 mM buffer such as histidine-based buffer,
1% stabilizer such as sucrose,
0.075% PS80,
pH 6.5;
   optionally, any of the co-formulations set forth herein further comprises GalNAc or GlcNAc, e.g., about 5% (w/v).

In an embodiment of the disclosure, a co-formulation of the present disclosure includes the antibody and iRNA and is in association with a further therapeutic agent, such as, for example, an anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, or methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor and/or an anti-thrombotic agent.

The term “in association with” indicates that a co-formulation is provided along with (2) one or more further therapeutic agents, such as methotrexate, which can be formulated into a single composition, e.g., for simultaneous delivery, or formulated separately into two or more compositions (e.g., a kit including each component, for example, wherein the further therapeutic agent is in a separate formulation). Components administered in association with each another can be administered to a subject at the same time or at a different time than when the other component is administered; for example, each administration may be given simultaneously (e.g., together in a single composition or essentially simultaneously during the same administration session) or non-simultaneously at one or more intervals over a given period of time. Moreover, the separate components administered in association with each another may be administered to a subject by the same or by a different route. Thus, the present disclosure includes co-formulations which are in association with a further therapeutic agent as well as methods of treating or preventing a disease or disorder associated with C5 (e.g., PNH, MG or CHAPLE) in a subject by administering to a subject in need thereof a co-formulation of the present disclosure in association with a further therapeutic agent.

The present disclosure includes co-formulations described herein wherein the concentration of the antibody and/or iRNA is ±10% the value shown; the concentration of surfactant is ±50% the value shown; and/or any of the other excipient concentrations (e.g., viscosity reducer, buffer, stabilizer) or pH are ±20% the value shown.

In an embodiment of the disclosure, a co-formulation of the present disclosure,
comprises about 0.04 to 0.17 micrograms/ml of beta-hexosaminidase (e.g., about 0.04; 0.05; 0.06; 0.06; 0.0605; 0.0605; 0.0605; 0.063; 0.07; 0.07; 0.0765; 0.078; 0.08; 0.14; 0.141; 0.15; 0.1525; 0.166; or 0.17 micrograms/ml (or no more than such an amount));
is a clear to slightly opalescent liquid;
is essentially free of visible particulates;
has a colorless to yellow color;
is characterized by a ratio of Cemdisiran concentration:Pozelimab concentration of about 1:1, 1:2, 1:3, 1:4, 1:5 or 2:3, e.g., wherein the viscosity if less than about 20 cP or less than about 30 cP at 20° C.;
has a Viscosity <30 cP (at 20° C.) (e.g., about 6, 10, 20 or 30 cP);
has an osmolality of 266-706 (e.g., about 266; 276; 286; 296; 306; 316; 326; 334; 336; 346; 356; 366; 376; 386; 396; 406; 416; 426; 436; 446; 456; 466; 476; 486; 496; 506; 516; 526; 536; 546; 556; 566; 576; 586; 596; 606; 616; 626; 636; 646; 656; 666; 676; 686; 696; or 706;) mOsm/kg;
has a density of about 1.1 or 1.061 g/ml;
has a pH of about 6.5±0.2;
Has a pH higher than 6 which results in a significant reduction in Cemdisiran degradation at 40° C., 25° C., and 2-8° C. relative to pH 6;
exhibits a Cemdisiran Purity (%) by dIPRP of about 90.5% at t=0; 91.1% after 1 month storage at 2-8° C.; 90.8% after 3 months storage at 2-8° C.; 90% after 6 months storage at 2-8° C.; 88.8% after 9 months storage at 2-8° C.; 88.7% after 12 months storage at 2-8° C.; 89% after 18 months storage at 2-8° C.; 89.4% after 24 months storage at 2-8° C.; and/or 87.4% after 36 months storage at 2-8° C., e.g., wherein the co-formulation was liquid (aqueous) during storage and includes 100 mg/ml Cemdisiran and 100 mg/ml Pozelimab (e.g., at pH 6.0); for example wherein the co-formulation includes about 60.5 ng/ml beta-hex;
exhibits a Cemdisiran Purity (%) by dIPRP of about 90.8% at t=0; 90.6% after 1 month storage at 2-8° C.; 90.5% after 3 months storage at 2-8° C.; 89.4% after 6 months storage at 2-8° C.; 88.3% after 9 months storage at 2-8° C.; 87.8% after 12 months storage at 2-8° C.; 87.8% after 18 months storage at 2-8° C.; and/or 87.4% after 24 months storage at 2-8° C. and/or about 85.4% after 36 months storage at 2-8° C.; e.g., wherein the co-formulation was liquid (aqueous) during storage and includes 75 mg/ml Cemdisiran and 150 mg/ml Pozelimab (e.g., at pH 6.0); for example wherein the co-formulation includes about 60.5 ng/ml beta-hex;
exhibits a Cemdisiran Single Strand Purity (%) by dIPRP of about 90.5% at t=0; 90.2% after 1 month storage at 25° C. and 60% RH (relative humidity); 87.8% after 3 months storage at 25° C. and 60% RH; 85.1% after 6 months storage at 25° C. and 60% RH; 90% after 0.5 months storage at 40° C. and 75% RH; 88.9% after 1 month storage at 40° C. and 75% RH; 85.8% after 3 months storage at 40° C. and 75% RH; e.g., wherein the co-formulation was liquid (aqueous) during storage and includes 100 mg/ml Cemdisiran and 100 mg/ml Pozelimab (e.g., at pH 6.0); for example wherein the co-formulation includes about 60.5 ng/ml beta-hex;
exhibits a Cemdisiran Single Strand Purity (%) by dIPRP of about 91.4% after about 48 hours agitation, and/or about 90.7% after about 4 freeze-thaw cycles e.g., wherein the co-formulation was liquid (aqueous) and includes 100 mg/ml Cemdisiran and 100 mg/ml Pozelimab (e.g., at pH 6.0);
exhibits a Cemdisiran Purity (%) by dIPRP of about 90.8% at t=0; 88.8% after 1 month storage at 25° C. and 60% RH; 85.9% after 3 months storage at 25° C. and 60% RH; 82.3% after 6 months storage at 25° C. and 60% RH; 88.9% after 0.5 months storage at 40° C. and 75% RH; 87.3% after 1 month storage at 40° C. and 75% RH; 82.3% after 3 months storage at 40° C. and 75% RH; e.g., wherein the co-formulation was liquid (aqueous) during storage and includes 75 mg/ml Cemdisiran and 150 mg/ml Pozelimab (e.g., at pH 6.0); for example wherein the co-formulation includes about 60.5 or 91 ng/ml beta-hex;
exhibits a Cemdisiran Purity (%) by dIPRP of about 90.7% after about 48 hours agitation, and/or about 91.2% after about 4 freeze-thaw cycles e.g., wherein the co-formulation was liquid (aqueous) during storage and includes 75 mg/ml Cemdisiran and 150 mg/ml Pozelimab (e.g., at pH 6.0);
exhibits no more (e.g., within 5%) increase in detectable high molecular weight species (HMW species) after 48 hours of agitation on an orbital shaker at 250 rpm in the presence of 0.025% non-ionic surfactant (e.g., polysorbate 80) than in the presence of 0.050, 0.075, 0.100, 0.125, 0.150, 0.175 or 0.200% (w/v); e.g., wherein the co-formulation includes 50 or 100 mg/mL Cemdisiran and 100 mg/mL Pozelimab, for example, 100 mg/mL Cemdisiran, 100 mg/mL Pozelimab, 50 mM arginine HCl, 30 mM histidine, 1% sucrose, X % PS80, pH 6.5, or 50 mg/mL Cemdisiran, 100 mg/mL Pozelimab, 90 mM arginine HCl, 30 mM histidine, 1% sucrose, X % PS80, pH 6.5 (wherein X is about 0.025% to about 0.2% (w/v), e.g., 0.075%);
exhibits a Cemdisiran purity (%) by dIPRP of about 90.9% at t=0; about 90.1% after 1 month of storage at 25° C., 60% RH; about 90.9% after 3 months of storage at 25° C., 60% RH; about 90.4% after 6 months of storage at 25° C., 60% RH; about 89.9% after 0.5 months of storage at 40° C., 75% RH; about 89.7% after 1 month of storage at 40° C., 75% RH; and/or about 89.5% after 3 months of storage at 40° C., 75% RH; e.g., wherein the co-formulation was liquid (aqueous) during storage and contained 50 mg/ml Cemdisiran and 100 mg/ml Pozelimab, pH6 with 5% GlcNAc; e.g., wherein the co-formulation includes about 78 ng/ml beta-hex;
exhibits a Cemdisiran purity (%) by dIPRP of about 90.8% at t=0; about 90.2% after 1 month of storage at 25° C., 60% RH; about 90.8% after 3 months of storage at 25° C., 60% RH; about 90.3% after 6 months of storage at 25° C., 60% RH; about 89.5% after 0.5 months of storage at 40° C., 75% RH; about 89.6% after 1 month of storage at 40° C., 75% RH; and/or about 89.1% after 3 months of storage at 40° C., 75% RH; e.g., wherein the co-formulation was liquid (aqueous) during storage and contained 50 mg/ml Cemdisiran and 100 mg/ml Pozelimab, pH6 with 5% GalNAc; e.g., wherein the co-formulation includes about 78 ng/ml beta-hex;
exhibits a Cemdisiran purity (%) by dIPRP of about 90.5% at t=0; about 89.9% after 1 month of storage at 25° C., 60% RH; about 90.8% after 3 months of storage at 25° C., 60% RH; about 90.4% after 6 months of storage at 25° C., 60% RH; about 90.1% after 0.5 months of storage at 40° C., 75% RH; about 89.6% after 1 month of storage at 40° C., 75% RH; and/or about 89.9% after 3 months of storage at 40° C., 75% RH; e.g., wherein the co-formulation was liquid (aqueous) during storage and contained 100 mg/ml Cemdisiran and 100 mg/ml Pozelimab, pH6 with 5% GlcNAc; e.g., wherein the co-formulation includes about 78 ng/ml beta-hex; and/or
exhibits a Cemdisiran purity (%) by dIPRP of about 91.1% at t=0; about 90% after 1 month of storage at 25° C., 60% RH; about 91% after 3 months of storage at 25° C., 60% RH; about 90.7% after 6 months of storage at 25° C., 60% RH; about 90% after 0.5 months of storage at 40° C., 75% RH; about 89.7% after 1 month of storage at 40° C., 75% RH; and/or about 89.9% after 3 months of storage at 40° C., 75% RH; e.g., wherein the co-formulation was liquid (aqueous) during storage and contained 100 mg/ml Cemdisiran and 100 mg/ml Pozelimab, pH 6 with 5% GalNAc; e.g., wherein the co-formulation includes about 78 ng/ml beta-hex;

Combination Therapy Dosing Regimens of Anti-C5 and C5 iRNA

The present disclosure includes methods that comprise administering to a subject in need thereof, with a disease or disorder or condition associated with C5, an anti-C5 antibody or antigen-binding fragment thereof in combination with a C5 iRNA (e.g., in the form of a co-formulation including both the antibody or fragment and the iRNA, e.g., as set forth herein) at a dosing amount and frequency that achieves a safe and effective therapeutic response (combination therapy of the present disclosure).

In some embodiments, the present disclosure relates to the administration of one or more doses of an anti-C5 antibody or antigen-binding fragment thereof (e.g., Pozelimab) in combination with one or more doses of a C5 iRNA (e.g., Cemdisiran). Preferably, the administration is in a co-formulation of the present disclosure (as discussed herein), e.g., 100:100 or 50:100 (Cemdisiran mg/ml:Pozelimab mg/ml)), for example in an injection volume of about 2 ml.

Generally, herein, a co-formulation including Cemdisiran and Pozelimab may be referred to in the following format: 100:100, 75:150 or 50:100. In such notation, when referring to such a co-formulation, the first number indicates the mg/ml of Cemdisiran and the second number indicates the mg/ml of Pozelimab.

A “dosing regimen” or “combination therapy dosing regimen” refers to a method for treating or preventing a disease or disorder or condition associated with C5 (preferably, PNH) including administering amounts of a combination therapy of the present disclosure at the frequencies as discussed herein.

For example, the present disclosure encompasses methods for administering an anti-C5 antibody or antigen-binding fragment thereof and a C5 iRNA comprising introducing the agents into the body of a subject, e.g., by injection such as by subcutaneous injection or intravenous infusion, for example, under a schedule according to any of the dosing regimens discussed herein (e.g., about 400 mg of the anti-C5 antibody or antigen-binding fragment (e.g., Pozelimab) subcutaneously about every 2-4 weeks (±3, 4, 5, 6 or 7 days) and about 200 mg of the iRNA (e.g., Cemdisiran) subcutaneously about every 4 weeks (±3, 4, 5, 6 or 7 days)).

Thus, the present disclosure provides a method for treating or preventing a C5-associated disease or disorder (for example, dry AMD or MG; preferably, PNH) in a subject in need thereof comprising administering to the subject an anti-C5 antibody or antigen-binding fragment thereof (“the anti-C5 Ab”) and a C5 iRNA according to the following:
(i) one or more doses of about 400 mg of the anti-C5 Ab (e.g., Pozelimab) subcutaneously, and about 200 mg of the C5 iRNA (e.g., Cemdisiran) subcutaneously;
(ii) about 400 mg of the anti-C5 Ab (e.g., Pozelimab) is administered subcutaneously about every 2 weeks, and about 200 mg of the C5 iRNA (e.g., Cemdisiran) is administered subcutaneously about every 4 weeks. Preferably, each 4 weeks (Q4W), a single injection of a co-formulation that includes the anti-C5 Ab and the C5 iRNA is administered and a separate additional injection of the anti-C5 Ab and not the C5 iRNA is administered on a biweekly basis (Q2W);
(iii) about 400 mg of the anti-C5 Ab (e.g., Pozelimab) is administered subcutaneously about every 4 weeks, and about 200 mg of the C5 iRNA (e.g., Cemdisiran) is administered subcutaneously about every 4 weeks, preferably in a single injection about every 4 weeks of a co-formulation that includes the anti-C5 Ab and the C5 iRNA; or
(iv) in a 50:100 co-formulation, about 4 ml that is administered subcutaneously, about 400 mg of the anti-C5 Ab (e.g., Pozelimab), and about 200 mg of the C5 iRNA (e.g., Cemdisiran) about every 4 weeks.

In an embodiment of the disclosure, the subject is administered, concurrently,
about 400 mg of the anti-C5 antibody or antigen-binding fragment (e.g., Pozelimab) subcutaneously about every 2-4 weeks (±3, 4, 5, 6 or 7 days); and
about 200 mg of the iRNA (e.g., Cemdisiran) subcutaneously about every 2-4 weeks (e.g., 4 weeks) (±3, 4, 5, 6 or 7 days).

The present disclosure also includes embodiments wherein the subject is administered concurrently:
about 400 mg of the anti-C5 antibody or antigen-binding fragment subcutaneously about every 2 weeks (±3, 4, 5, 6 or 7 days); and
every 4 weeks (±3, 4, 5, 6 or 7 days) a 200 mg dose of C5 iRNA subcutaneously.

The present disclosure also includes embodiments wherein the subject is administered concurrently:
about 400 mg of the anti-C5 antibody or antigen-binding fragment subcutaneously about every 4 weeks (±3, 4, 5, 6 or 7 days); and
every 4 weeks (±3, 4, 5, 6 or 7 days) a 200 mg dose of C5 iRNA subcutaneously (may be referred to herein as “Pozelimab Q4W and Cemdisiran” or ““Pozelimab 400 mg SC Q4W+Cemdisiran 200 mg SC Q4W).
   Switch from Pozelimab Monotherapy

A dosing regimen, e.g., for treatment of a disease or disorder or condition associated with C5 such as PNH, including the anti-C5 and C5 iRNA, for a subject who has previously received Pozelimab monotherapy, e.g., as set forth herein, may be referred to herein as a “Pozelimab Monotherapy Switch” regimen.

In an embodiment of the invention, the regimen is as follows: On day 1 (7 to 8 days after the last dose of Pozelimab monotherapy) or, when the next dose of pozelimab monotherapy is due, subjects start receiving either
(1) Pozelimab 400 mg SC every 4 weeks (Q4W) and Cemdisiran 200 mg SC Q4W; or
(2) Pozelimab 400 mg SC every 2 weeks (Q2W) and Cemdisiran 200 mg SC Q4W.

Pozelimab monotherapy include treatment of a disease or disorder or condition associated with C5 (preferably, PNH) with Pozelimab as the only C5-specific inhibitor or, more specifically, anti-C5 antibody or antigen-binding fragment (e.g., not with both Pozelimab and Eculizumab). In an embodiment of the present disclosure, before receiving a combination therapy as discussed herein, 400 mg anti-C5 antibody or antigen-binding fragment SC about every 2, 3 or 4 weeks and about 400 mg C5 iRNA SC about every 4 weeks, the subject has received a dosing regimen according to the following:
(i) one or more doses of about 30 mg/kg of the antigen-binding protein intravenously (IV); then
(ii) one or more doses (e.g., weekly) of about 800 mg of Pozelimab, subcutaneously (SC) [may be referred to as maintenance phase]; e.g., wherein the subject suffers from PNH;
or,
(a) one or more doses, preferably, only one dose, of about 30 mg/kg of Pozelimab intravenously (IV); then
(b) one or more doses (e.g., weekly) of 10 mg/kg Pozelimab, subcutaneously (SC) [may be referred to as maintenance phase], e.g., wherein the subject suffers from CHAPLE
or,
(a) one or more doses, preferably, only one dose, of about 30 mg/kg of Pozelimab intravenously (IV); then
(b) one or more doses (e.g., weekly) of Pozelimab, subcutaneously (SC), according to the following:
   for body weight (BW)<about 10 kg: about 125 mg;
   for BW≥10 kg and <about 20 kg: about 200 mg;
   for BW≥20 kg and <about 40 kg: about 350 mg;
   for BW≥40 kg and <about 60 kg: about 500 mg; and
   for BW≥60 kg: about 800 mg;
[may be referred to as maintenance phase]
   e.g., wherein the subject suffers from CHAPLE;
wherein, the subject has received one or more of such doses and transitions to the combination therapy at any point (e.g., from the maintenance phase). In an embodiment of the present disclosure, the subject begins receiving the combination of the anti-C5 and C5 iRNA on the day that the next dose of Pozelimab monotherapy is due to be administered and ceases the monotherapy at that time as well. For example, wherein the subject transitions from a Pozelimab monotherapy and receives the first dose of the combination after receiving:
(i) one or more doses of about 800 mg of Pozelimab subcutaneously (SC);
(ii) one or more doses of about 30 mg/kg of Pozelimab intravenously (IV);
(iii) one or more doses of about 125 mg of Pozelimab subcutaneously (SC);
(iv) one or more doses of about 200 mg of Pozelimab subcutaneously (SC);
(v) one or more doses of about: 350 mg of Pozelimab subcutaneously (SC);
(vi) one or more doses of about: 500 mg of Pozelimab subcutaneously (SC);
(vii) one or more doses of about 800 mg of Pozelimab subcutaneously (SC);
(viii) one or more doses of 10 mg/kg Pozelimab subcutaneously (SC),
e.g., wherein the first dose of the combination is received on the date when dose labeled (i), (ii), (iii), (iv), (v), (vi), (vii) or (viii) would have been due or about 1 week after the last dose of the Pozelimab monotherapy was received.

In an embodiment of the present disclosure, subcutaneous Pozelimab monotherapy doses are administered in a formulation comprising about:
161-274 mg/ml or more Pozelimab, and
a pharmaceutically acceptable carrier comprising: a buffer; L-arginine; water; and, optionally, an oligosaccharide (e.g., sucrose, mannitol, dextrose, glycerol, TMAO (trimethylamine N-oxide), trehalose, ethylene glycol, glycine betaine, xylitol or sorbitol); and optionally, a non-ionic detergent (e.g., polysorbate-20, polysorbate-80),
   with a pH of up to about 5.8, 6.1 or 5.5-6.1; and, a viscosity, at 20° C., of about 6.8, about 9.6, about 11.9, about 13.2, about 16.7, about 20.6, about 33.0, about 48.4, about 13.2-16.7 or about 6.8-48.4. Intravenous doses of Pozelimab monotherapy can be administer after introducing the formulation into an aqueous intravenous solution (e.g., 0.9% Normal Saline).

### Complement Inhibitor Naive

Naïve or complement inhibitor naïve patients have not ever or not recently (e.g., not in the last 1, 2, 3, 4, 5 or 6 months or for at least about 4 or 5 half-lives of the last complement inhibitor they received) received complement inhibitor therapy (e.g., Eculizumab, Ravulizumab, Pozelimab).

In an embodiment of the disclosure, a complement inhibitor naïve subject is treated for a disease or disorder or condition associated with C5 (preferably, PNH) by a method including administering:
On day 1: Pozelimab in a single loading dose of 30 mg/kg intravenous (IV) and (e.g., followed, optionally, by a delay of at least about 30 minutes) 400 mg subcutaneous (SC), and Cemdisiran 200 mg SC (the combination maintenance dose);
Starting on day 29 or about 4 weeks later: Pozelimab 400 mg SC every 4 weeks (q4W) and Cemdisiran 200 mg SC q4W (e.g., in a Cemdisiran/Pozelimab co-formulation).

In an embodiment of the disclosure, a complement inhibitor naïve subject is treated for a disease or disorder or condition associated with C5 (preferably, PNH) by a method including administering:
(i) on about day 1, a single loading dose of about 30 mg/kg intravenous (IV) an anti-C5 antibody or antigen-binding fragment, preferably Pozelimab, followed by about 400 mg subcutaneous (SC) of the anti-C5 antibody or fragment and about 200 mg SC of C5 iRNA, preferably Cemdisiran; and
(ii) starting on about day 29, an about 400 mg SC dose of the anti-C5 antibody or fragment Q4W and an about 200 mg SC dose of the C5 iRNA Q4W.

During the transition of therapy from Eculizumab or Ravulizumab to a therapy including the anti-C5 Ab (e.g., Pozelimab) and C5 iRNA (e.g., Cemdisiran), an additional dose of Pozelimab (e.g., a dose of 30 or 60 mg/kg IV) can be administered, e.g., under circumstances such as if there is a suspicion that an adverse event (AE) potentially due to large drug-target-drug (DTD) immune complexes may occur or has occurred and/or if systemic corticosteroids are administered for a type III hypersensitivity reaction. This additional dose will establish conditions of Pozelimab excess in the circulation and thereby minimize the risk of further formation of immune complexes.

Switch from Eculizumab Therapy

A dosing regimen for a subject who has previously received Eculizumab, e.g., as set forth above, may be referred to herein as an “Eculizumab Switch” regimen. Preferably, the subject is being treated for a disease or disorder or condition associated with C5, such as PNH.

In an embodiment of the invention, the Eculizumab Switch regimen has a lead-in loading phase and a switch phase as follows:

### Lead-In

Initially, subjects remain on Eculizumab background treatment at their usual dose/frequency, and Cemdisiran alone is introduced as follows:
On day 1 (the day of subject's scheduled Eculizumab administration, preferably wherein the subject is on a q2w maintenance regimen of Eculizumab): Cemdisiran 200 mg SC and Eculizumab >900 mg IV (subject's usual dose). Note: Eculizumab may be administered up to 2 days after Cemdisiran if not administered with Cemdisiran on day 1;
On day 15 (+2 days), for subjects on Eculizumab q14 days (labeled dose regimen): Labeled Eculizumab dose [for subjects on Eculizumab more frequently than q14 days: patients are dosed within 2 days of their usual planned dose];

### Switch

On day 29 (or week 4 (counting from day 1) or about 2 weeks later or when the next Eculizumab dose is due or about 1-2 half-lives of Eculizumab): Pozelimab 60 mg/kg IV loading dose, and (e.g., followed, optionally, by a delay of at least about 30 minutes) Pozelimab 400 mg SC and Cemdisiran 200 mg SC (e.g., in a Cemdisiran/Pozelimab co-formulation); and
Starting on day 57 (or week 8; or about 4 weeks later): Pozelimab 400 mg SC and Cemdisiran 200 mg SC q4W maintenance regimen (±7 days) (e.g., in a Cemdisiran/Pozelimab co-formulation) [may be referred to as maintenance phase]

In an embodiment of the invention, the Eculizumab half-life (e.g., in a subject having PNH) is about 4, 5, 6, 7, 8, 9, 10, 11, 12, 13, 14, 15, 16, 17, 18, 19, 20 or 21 days, e.g., about 11 days (Wijnsma et al., Pharmacology, Pharmacokinetics and Pharmacodynamics of Eculizumab, and Possibilities for an Individualized Approach to Eculizumab. Clin Pharmacokinet. 2019 July; 58(7):859-874; AI-Ani et al., Eculizumab in the management of paroxysmal nocturnal hemoglobinuria: patient selection and special considerations. Ther Clin Risk Manag. 2016 Aug. 1; 12:1161-70).

In an embodiment of the present disclosure, the dosing regimen includes:
(i) an initial dose (on day 1; e.g., on the day the next dose of Eculizumab is due or up to 2 days prior) of Eculizumab (at the subject's usual dose, e.g., ≥about 900 mg) intravenously and about 200 mg of the C5 iRNA, subcutaneously;
(ii) a second dose of the Eculizumab (at the subject's usual dose, e.g., ≥about 900 mg) up to about 14 days (+2 days) later;
(iii) on about day 29 (week 4): the anti-C5 Ab at a dose of about 60 mg/kg intravenously (IV), followed by the anti-C5 Ab at a dose of about 400 mg subcutaneously and the C5 iRNA at a dose of about 200 mg subcutaneously; and
(iv) on day 57 (week 8) and forward, the anti-C5 Ab at a dose of about 400 mg subcutaneously about every 4 weeks (±3, 4, 5, 6 or 7 days) and the C5 iRNA at a dose of about 200 mg subcutaneously about every 4 weeks (±3, 4, 5, 6 or 7 days).

A prescribed dosing regimen (e.g., for subjects 18 years of age or older) for treatment of PNH with Eculizumab is as follows:
600 mg weekly for the first 4 weeks, followed by
900 mg for the fifth dose 1 week later, then
900 mg every 2 weeks thereafter.

A prescribed dosing regimen for treatment of aHUS with Eculizumab is as follows:
900 mg weekly for the first 4 weeks, followed by
1200 mg for the fifth dose 1 week later, then
1200 mg every 2 weeks thereafter.

A prescribed dosing regimen of Eculizumab for treatment of generalized myasthenia gravis or neuromyelitis optica spectrum is as follows:
900 mg weekly for the first 4 weeks, followed by
1200 mg for the fifth dose 1 week later, then
1200 mg every 2 weeks thereafter.
   See Soliris® Prescribing Information. In an embodiment of the present disclosure a subject has previously received Eculizumab treatment for at least 12 weeks.

In an embodiment of the present disclosure, Eculizumab is administered to a subject in a dose taken from a pharmaceutical formulation comprising 300 mg of Eculizumab, polysorbate 80 (6.6 mg) (vegetable origin), sodium chloride (263.1 mg), sodium phosphate dibasic (53.4 mg), sodium phosphate monobasic (13.8 mg), and Water for Injection, USP, at pH 7 and in a volume of 30 mL.

### See Soliris® Prescribing Information.

Switch from Ravulizumab Therapy

A dosing regimen for a subject who has previously received Ravulizumab, e.g., as set forth above, may be referred to herein as an “Ravulizumab Switch” regimen. Preferably, the subject is being treated for a disease or disorder or condition associated with C5 such as PNH.

In an embodiment of the disclosure, a Ravulizumab switch regimen is as follows:
On day 1 (4 weeks after the last administration of Ravulizumab, preferably wherein the subject is receiving a q8w Ravulizumab maintenance regimen): Cemdisiran 200 mg SC;
On day 29 or 4 weeks later or 8 weeks after the last administration of Ravulizumab or after 1-2 half-lives of Ravulizumab: Pozelimab 30 mg/kg or 60 mg/kg single IV loading dose, and (e.g., followed, optionally, by a delay of at least about 30 minutes) Pozelimab 400 mg SC and Cemdisiran 200 mg SC (e.g., in a Cemdisiran/Pozelimab co-formulation); and
Starting on Day 57 or 4 weeks later or after 1-2 half-lives of Ravulizumab: Start Pozelimab 400 mg SC q4W and Cemdisiran 200 mg SC (e.g., in a Cemdisiran/Pozelimab co-formulation) q4W maintenance regimen.

In an embodiment of the invention, the Ravulizumab half-life (e.g., in a subject having PNH) is about 32 days (Stern et al., Ravulizumab: a novel C5 inhibitor for the treatment of paroxysmal nocturnal hemoglobinuria. Ther Adv Hematol. 2019 Sep. 10; 10:2040620719874728; Lee et al., Ravulizumab (ALXN1210) vs eculizumab in adult patients with PNH naive to complement inhibitors: the 301 study. Blood. 2019 Feb. 7; 133(6):530-539; Lee et al., Immediate, complete, and sustained inhibition of C5 with ALXN1210 reduces complement-mediated hemolysis in patients with paroxysmal nocturnal hemoglobinuria (PNH): interim analysis of a dose-escalation study [abstract]. Blood. 2016; 128(22). Abstract 2428).

In an embodiment of the disclosure, a subject is treated for a disease or disorder or condition associated with C5 (preferably, PNH), where the subject previously received Ravulizumab (e.g., according to the prescribed dosing regimen) and is being switched to a treatment regimen with a different anti-C5 antibody or antigen-binding fragment (the anti-C5 Ab), preferably Pozelimab, and a C5 iRNA (the C5 iRNA), preferably Cemdisiran, is administered:
(i) on day 1 (e.g., 4 weeks (17 days) or 26 days (±7 days) or 27 days (±7 days) or 28 days (17 days) after the last administration of Ravulizumab), the C5 iRNA 200 mg SC;
(ii) on about day 29, a single IV loading dose of the anti-C5 Ab at 30 mg/kg or 60 mg/kg. This IV loading dose may be followed by a 30 minute observation period. The IV dose is followed by an additional loading dose of the anti-C5 Ab 400 mg SC and the C5 iRNA 200 mg SC;
(iii) on about day 57, the anti-C5 Ab 400 mg SC and the C5 iRNA 200 mg SC maintenance regimen initiates and may be followed,
(iv) about every 4 weeks thereafter with administering the anti-C5 Ab 400 mg SC and the C5 iRNA 200 mg SC.

The day 1 Ravulizumab loading dose can be according to the patient's weight (>40 kg to <60 kg, 2400 mg IV; >60 kg to <100 kg, 2700 mg IV; 2100 kg, 3000 mg IV). The first maintenance dose that is administered 2 weeks after the loading dose as follows: (>40 kg to <60 kg, 3000 mg IV; >60 kg to <100 kg, 3300 mg IV; 100 kg, 3600 mg IV). Thereafter, the maintenance doses should be administered IV Q8W (±7 days).

A Ravulizumab subcutaneous maintenance dose is 490 mg once weekly in adult patients greater than or equal to 40 kg body weight, e.g., with PNH or aHUS. The subcutaneous dosing schedule is allowed to occasionally vary by ±1 day of the scheduled dose day, but the subsequent dose should be administered according to the original schedule.

A prescribed dosing regimen for treatment of PNH with Ravulizumab is as follows:
For patients with a body weight of 5 to less than 10 kg, a loading dose of 600 mg and a maintenance dose of 300 mg every 4 weeks;
For patients with a body weight of 10 to less than 20 kg, a loading dose of 600 mg and a maintenance dose of 600 mg every 4 weeks;
For patients with a body weight of 20 to less than 30 kg, a loading dose of 900 mg and a maintenance dose of 2,100 mg every 8 weeks;
For patients with a body weight of 30 to less than 40 kg, a loading dose of 1200 mg and a maintenance dose of 2,700 mg every 8 weeks;
For patients with a body weight of 40 to less than 60 kg, a loading dose of 2,400 mg and a maintenance dose of 3,000 mg every 8 weeks;
For patients with a body weight of 60 to less than 100 kg, a loading dose of 2,700 mg and a maintenance dose of 3,300 mg every 8 weeks; and
For patients with a body weight of 100 or greater kg, a loading dose of 3,000 mg and a maintenance dose of 3,600 mg every 8 weeks.

### See Ultomiris® Prescribing Information.

Subcutaneous Ravulizumab maintenance doses may be 490 mg once weekly in adult patients greater than or equal to 40 kg body weight with PNH. Patients not currently on Ravulizumab or Eculizumab treatment with a body weight of >40 kg at treatment start may initiate the subcutaneous doses of Ravulizumab about 2 weeks after the intravenous Ravulizumab loading dose. Patients currently treated with Eculizumab with a body weight of >40 kg at time of next scheduled Eculizumab dose may initiate the subcutaneous doses of Ravulizumab about 2 weeks after the intravenous Ravulizumab loading dose. Patients currently treated with Ravulizumab intravenous (IV) administration may initiate the subcutaneous doses of Ravulizumab about 8 weeks after the last intravenous Ravulizumab maintenance dose. In an embodiment of the present disclosure, the subject has previously received Ravulizumab treatment for at least 24 weeks.

TABLE C
Summary of Eculizumab and Ravulizumab Switch Regimens

| | Patients switching from Eculizumab | Patients switching from Ravulizumab |
|---|---|---|
| Day 1 of Switch | Introduction of Cemdisiran: 200 mg SC | Ravulizumab IV at the labeled |
| | along with Eculizumab IV* | weight-based dose |
| rcDay 15 (week 2) | Eculizumab IV | |
| Day 29 (week 4) | Pozelimab 60 mg/kg IV loading dose | Introduction of Cemdisiran: |
| | followed on the same day by Pozelimab | Cemdisiran 200 mg SC |
| | 400 mg SC and Cemdisiran 200 mg SC and | |
| | thereafter, Pozelimab 400 mg SC and | |
| | Cemdisiran 200 mg SC Q4W | |
| Day 57 (week 8) | (see above) | Pozelimab 60 mg/kg IV loading dose |
| | | followed on the same day by Pozelimab |
| | | 400 mg SC and Cemdisiran 200 mg SC and |
| | | thereafter, Pozelimab 400 mg SC and |
| | | Cemdisiran 200 mg SC Q4W |

*For example, 900 mg Eculizumab (IV)

As discussed, the subject may have been previously receiving Pozelimab monotherapy, e.g., at a dosage of about 800 mg subcutaneously (SC) every 1, 2, 3 or 4 weeks (which may have been preceded by a loading dose of Pozelimab, e.g., intravenously), or Ravulizumab or Eculizumab, e.g., according to a prescribed dosing regimen. Patients who have received Pozelimab monotherapy, Ravulizumab or Eculizumab previously may be in any phase of the prescribed dosing regimen of the antibody before switching to a combination therapy of the present disclosure. For example, the subject may have received one or more loading doses and/or one or more maintenance doses of Eculizumab. In an embodiment of the invention, prior to or on the same day as initiating treatment with a monthly regimen of 400 mg Pozelimab and 200 mg Cemdisiran, when the subject is switching from Eculizumab or Ravulizumab or another anti-C5 antibody or antigen-binding fragment thereof, the subject receives an intravenous loading dose of Pozelimab (e.g., 30 mg/kg or 60 mg/kg) and/or a single SC dose of Cemdisiran (e.g., 200 mg). The transition period mitigates the risk for the formation of large DTD (drug-target-drug) immune complexes of Eculizumab-C5-Pozelimab during the switch from Eculizumab or Ravulizumab-C5-Pozelimab during the switch from Ravulizumab to the Pozelimab+Cemdisiran combination.

Pozelimab binds C5 non-competitively with eculizumab, and thus has the potential to form heteromeric complexes including large DTD immune complexes. In vitro, neither Pozelimab nor Eculizumab individually form higher-order multimers larger than a 1:2 mAb:C5 complex with C5. Pozelimab was added to pre-formed in-house Eculizumab:C5 complexes under conditions of excess Pozelimab (5:1:1 Pozelimab:in-house eculizumab:C5) and equimolar amounts of total mAb to C5 (1:1:2 Pozelimab:in-house eculizumab:C5). Under conditions of Pozelimab excess, the majority of samples (˜86%) had free antibody and either trimeric or pentameric complexes (2:1 or 3:2 mAb:C5 molar ratios, respectively), with the remainder comprising large HMW complexes. At an equimolar ratio of total mAb and C5, the majority of the samples (˜86%) had large HMW complexes larger than pentamers. While Eculizumab and Pozelimab in combination were able to form heteromeric complexes with C5, the presence of excess Pozelimab reduced the formation of large higher order immune complexes relative to conditions where total mAb and C5 were present at equimolar concentrations.

When switching from a regimen that includes an antibody that does not compete significantly with Pozelimab for binding to C5 (a non-competing antibody or antigen-binding fragment thereof (N/C Ab), e.g., Eculizumab or Ravulizumab), a transition period is designed to mitigate the potential risk for the formation of large DTD immune complexes of, for example, Eculizumab-C5-Pozelimab during the switch from Eculizumab to the Pozelimab/Cemdisiran combination. The transition period can include a lead-in Cemdisiran dose followed by a high higher IV loading dose of Pozelimab (60 mg/kg) than is used in treatment naïve patients (30 mg/kg). The initial dose of Cemdisiran (e.g., occurring on day 1) reduces C5 production and, thereby, the circulating level of total C5 available for potential large DTD complex formation prior to the introduction of Pozelimab. To further minimize the risk, the 60 mg/kg IV loading dose of Pozelimab establishes a high Pozelimab:Eculizumab molar ratio. This excess concentration of Pozelimab reduces the formation of higher-order DTD immune complexes, relative to equimolar molar concentrations of total antibody and C5, by assuring saturation of the C5 binding sites by Pozelimab. This IV loading dose results in a molar ratio of Pozelimab to Eculizumab of approximately 17:1, based on a reported mean trough concentrations of Eculizumab of 97 mg/L (Soliris® Eculizumab (Prescribing information)) and the predicted concentration of Pozelimab.

An additional dose of anti-C5 antibody or antigen-binding fragment, preferably Pozelimab, of about 30 mg/kg IV, can be included in circumstances such as if there is a suspicion of an adverse event [AE] potentially due to large DTD (drug-target-drug) immune complexes and/or if systemic corticosteroids are administered for a type III hypersensitivity reaction. This additional dose will likely establish conditions of Pozelimab excess in the circulation and thereby minimize the risk of further formation of immune complexes.

Thus, the present disclosure includes:
methods for reducing the likelihood of formation of large DTD complexes and/or establishing an excess of Pozelimab (relative to that of a N/C antibody such as Eculizumab or Ravulizumab, e.g., a ratio of >1:1::Pozelimab: N/C Ab (greater than equimolar), for example, about 17:1::Pozelimab: N/C Ab) in the body of a subject prior to initiation of a regimen including Cemdisiran and Pozelimab (as set forth herein, e.g., Pozelimab 400 mg SC Q4W and Cemdisiran 200 mg SC Q4W), and
methods for treating or preventing a C5-associated disease or disorder (preferably, PNH) in a subject in need thereof comprising administering to the subject a therapeutically effective amount of an Pozelimab and C5 iRNA (preferably Cemdisiran), wherein the subject has previously received a treatment regimen including an antibody or antigen-binding fragment thereof that does not compete for binding to C5 with Pozelimab (a non-competing antibody or antigen-binding fragment thereof (N/C Ab), e.g., Eculizumab or Ravulizumab), wherein the subject is administered:
(1) a dose of C5 iRNA (e.g., Cemdisiran) after a dose of N/C Ab (e.g., Eculizumab or Ravulizumab), but before the first dose of Pozelimab (e.g., Pozelimab IV (e.g., 60 mg/kg) and/or Pozelimab 400 mg SC Q4W and Cemdisiran 200 mg SC Q4W);
or
(1) a dose of C5 iRNA (e.g., Cemdisiran) after a dose of N/C Ab, but before the first dose of Pozelimab; then
(2) an intravenous loading dose of Pozelimab (e.g., 60 mg/kg) which is the first dose of Pozelimab;
or
(1) an intravenous loading dose of Pozelimab (e.g., 60 mg/kg) along with a dose of Pozelimab 400 mg SC Q4W and Cemdisiran 200 mg SC Q4W; then
(2) starting about 4 weeks thereafter (and continuing every 4 weeks thereafter), Pozelimab 400 mg SC Q4W and Cemdisiran 200 mg SC Q4W;
or
(1) a dose of C5 iRNA (e.g., Cemdisiran) after a dose of the N/C Ab has been administered and about 4 weeks before an intravenous loading dose of Pozelimab (e.g., 60 mg/kg) along with a dose of Pozelimab 400 mg SC Q4W and Cemdisiran 200 mg SC Q4W; then
(2) starting about 4 weeks thereafter (and continuing every 4 weeks thereafter), Pozelimab 400 mg SC Q4W and Cemdisiran 200 mg SC Q4W;
or
(1) a dose of C5 iRNA (e.g., Cemdisiran) and the non-competing antibody or fragment (N/C Ab) on the day the dose of N/C Ab is due;
(2) the next dose of N/C Ab on the day such dose is due;
(3) after about 1-2 half-lives of the N/C Ab or when the next dose of N/C Ab would be due, a Pozelimab IV loading dose (e.g., 60 mg/kg), and Pozelimab 400 mg SC and Cemdisiran 200 mg SC;
(4) starting 4 weeks thereafter (and continuing every 4 weeks thereafter), Pozelimab 400 mg SC Q4W and Cemdisiran 200 mg SC Q4W;
or
(1) following about 1-2 half-lives of the N/C Ab from the last dose thereof; or, on the day the next dose of N/C Ab is due; or, after half of the interval between doses has elapsed since the last dose of N/C Ab, a dose of C5 iRNA (e.g., Cemdisiran);
(2) following about another 1-2 half-lives of the N/C Ab, Pozelimab IV loading dose (e.g., 60 mg/kg), Pozelimab 400 mg SC and Cemdisiran 200 mg SC; and
(3) starting 4 weeks thereafter (and continuing every 4 weeks thereafter), Pozelimab 400 mg SC Q4W and Cemdisiran 200 mg SC Q4W.

Generally, the initial dose or doses that are non-recurring may be referred to as “loading” doses and subsequent doses that are recurring may be referred to as “maintenance” doses.

In an embodiment of the invention, a large DTD complex refers to a complex larger that an pentameric complex (e.g., 2:1 or 3:2::mAb:C5 molar ratio) or a complex having a molecular weight of 1000 kDa or more.

In an embodiment of the invention, an excess of Pozelimab relative to an N/C Ab such as Eculizumab or Ravulizumab refers to a molar excess of greater than 1:1::Pozelimab:N/C Ab (e.g., 17:1).

A dosing regimen including monthly doses of both anti-C5 antibody or antigen-binding fragment thereof (e.g., Pozelimab; e.g., about 400 mg) and C5 iRNA (e.g., Cemdisiran; e.g., about 200 mg) may be referred to as the q4w or Q4W regimen.

A dosing regimen including doses (e.g., of about 400 mg) of anti-C5 antibody or antigen-binding fragment thereof every 2 weeks and monthly doses (e.g., of about 200 mg) of C5 iRNA may be referred to as the q2w or Q2W regimen.

The term “4 weeks” or a “month”, in an embodiment of the invention, refers to about 28, 29 or 30 days (±3, 4, 5, 6 or 7 days).

The term “2 weeks”, in an embodiment of the invention, refers to about 14 days (±3, 4, 5, 6 or 7 days).

Anti-C5 antibody or antigen-binding fragment thereof 400 mg SC Q4W refers to administration of about 400 mg of the antibody or fragment (e.g., Pozelimab) subcutaneously about every month, 4 weeks or 28 days (±3, 4, 5, 6 or 7 days).

Anti-C5 antibody or antigen-binding fragment thereof 400 mg SC Q2W refers to administration of about 400 mg of the antibody or fragment (e.g., Pozelimab) subcutaneously about every 2 weeks or 14 days (±3, 4, 5, 6 or 7 days).

C5 iRNA 200 mg SC Q4W refers to administration of 200 mg of the iRNA (e.g., Cemdisiran) subcutaneously about every 4 weeks or 28 days (±3, 4, 5, 6 or 7 days).

As set forth herein, any dosing episode (e.g., which involves multiple doses of drugs), may be followed by a 30 minute to 2-hour observation period after the last administration or for however long, in the judgment of the treating physician, no adverse events are likely to occur acutely. Typically, on a given day when a subject receives an intravenous dose and one or more subcutaneous doses, the intravenous dose is given first; however, the scope of the present disclosure includes embodiments wherein the doses are given in any order, e.g., SC then IV then SC.

In an embodiment of the disclosure, a subject receiving the combination therapy of anti-C5 Ab and C5 iRNA achieves or achieves and maintains while receiving the therapy one or more of the following:
Hemoglobin stabilization;
Does not receive a red blood cell transfusion;
Does not accumulate detectable and/or clinically significant quantities of drug-target-drug complexes (e.g., Pozelimab-C5-Pozelimab);
Does not accumulate detectable and/or clinically significant quantities of Eculizumab-C5-Pozelimab complex, e.g., if subject switched from Eculizumab therapy to combination therapy;
Does not experience an adverse event due to large DTD complexes, e.g., rash, fever, malaise, rash, or polyarthralgia;
Has no decrease in hemoglobin >2 g/dL;
Does not experience breakthrough hemolysis; CH50 levels in blood are fully suppressed relative to baseline before treatment and/or during any breakthrough hemolysis event;
Improvement in fatigue, relative to before treatment;
>5 point improvement in FACIT-Fatigue score relative to before treatment;
Improvement in physical functioning score on the European;
Organization for Research and Treatment of Cancer: Quality-of-Life Questionnaire; core 30 items (EORTC QLQ-C30)) relative to before treatment;
Improvement in GHS/QoL (global health status/QOL scale (GHS)), relative to before treatment;
Reduction in lactate dehydrogenase (LDH) levels relative to before treatment;
Achievement of LDH≤1.5× upper limit of normal (ULN) relative to before treatment;
Achievement and maintenance of LDH≤1.0×ULN;
A reduction in blood bilirubin levels relative to before treatment;
A reduction in reticulocyte count relative to before treatment;
A reduction in alternative pathway hemolytic activity assay (AH50) relative to before treatment;
A reduction in PNH erythrocytes and/or granulocytes relative to before treatment;
Improvement in fatigue, shortness of breath, muscle weakness, headache, abdominal, pain, pain in back/legs, chest discomfort, difficulty sleeping, difficulty thinking clearly, and/or difficulty swallowing relative to before treatment;
Improvement in renal function as measured by estimated glomerular filtration rate (eGFR) relative to before treatment;
Reduction in blood free hemoglobin relative to before treatment; and/or
Increase in haptoglobin level relative to before treatment.

In an embodiment of the disclosure, a subject receiving the combination therapy of anti-C5 Ab and C5 iRNA achieves or achieves and maintains while receiving the therapy one or more of the following:
A gain in FACIT-Fatigue score of about 13 when on the q4w regimen; or a gain of about 11 when on the q2w regimen, e.g., by about 2 weeks from the start of treatment;
A gain in FACIT-Fatigue score of about 8 when on the q4w regimen; or a gain of about 8 when on the q2w regimen, e.g., by about 4 weeks from the start of treatment;
A gain in FACIT-Fatigue score of about 11 when on the q4w regimen; or a gain of about 7 when on the q2w regimen, e.g., by about 8 weeks from the start of treatment;
A gain in FACIT-Fatigue score of about 11 when on the q4w regimen; or a gain of about 8 when on the q2w regimen, e.g., by about 12 weeks from the start of treatment;
A gain in FACIT-Fatigue score of about 12 when on the q4w regimen; or a gain of about 9 when on the q2w regimen, e.g., by about 16 weeks from the start of treatment;
A gain in FACIT-Fatigue score of about 11 when on the q4w regimen; or a gain of about 4 when on the q2w regimen, e.g., by about 20 weeks from the start of treatment;
A gain in FACIT-Fatigue score of about 12 when on the q4w regimen; or a gain of about 11 when on the q2w regimen, e.g., by about 24 weeks from the start of treatment;
A gain in FACIT-Fatigue score of about 11 when on the q4w regimen; or a gain of about 9 when on the q2w regimen, e.g., by about 28 weeks from the start of treatment;
A gain in EORTC-QLQ-C30 physical functioning score of about 23 when on the q4w regimen or of about 14 when on the q2w regimen, e.g., at about 2 weeks from the initiation of treatment;
A gain in EORTC-QLQ-C30 physical functioning score of about 19 when on the q4w regimen or of about 13 when on the q2w regimen, e.g., at about 4 weeks from the initiation of treatment;
A gain in EORTC-QLQ-C30 physical functioning score of about 22 when on the q4w regimen or of about 14 when on the q2w regimen, e.g., at about 8 weeks from the initiation of treatment;
A gain in EORTC-QLQ-C30 physical functioning score of about 20 when on the q4w regimen or of about 17 when on the q2w regimen, e.g., at about 12 weeks from the initiation of treatment;
A gain in EORTC-QLQ-C30 physical functioning score of about 19 when on the q4w regimen or of about 14 when on the q2w regimen, e.g., at about 16 weeks from the initiation of treatment;
A gain in EORTC-QLQ-C30 physical functioning score of about 23 when on the q4w regimen or of about 11 when on the q2w regimen, e.g., at about 20 weeks from the initiation of treatment;
A gain in EORTC-QLQ-C30 physical functioning score of about 20 when on the q4w regimen or of about 15 when on the q2w regimen, e.g., at about 24 weeks from the initiation of treatment;
A gain in EORTC-QLQ-C30 physical functioning score of about 24 when on the q4w regimen or of about 20 when on the q2w regimen, e.g., at about 28 weeks from the initiation of treatment;
A gain in EORTC-QLQ-C30 GHS/QoL score of about 15 when on the q4w regimen, or of about 14 when on the q2w regimen, e.g., by about 2 weeks from treatment initiation;
A gain in EORTC-QLQ-C30 GHS/QoL score of about 10 when on the q4w regimen, or of about 15 when on the q2w regimen, e.g., by about 4 weeks from treatment initiation;
A gain in EORTC-QLQ-C30 GHS/QoL score of about 9 when on the q4w regimen, or of about 9 when on the q2w regimen, e.g., by about 8 weeks from treatment initiation;
A gain in EORTC-QLQ-C30 GHS/QoL score of about 11 when on the q4w regimen, or of about 12 when on the q2w regimen, e.g., by about 12 weeks from treatment initiation;
A gain in EORTC-QLQ-C30 GHS/QoL score of about 12 when on the q4w regimen, or of about 8 when on the q2w regimen, e.g., by about 16 weeks from treatment initiation;
A gain in EORTC-QLQ-C30 GHS/QoL score of about 17 when on the q4w regimen, or of about 6 when on the q2w regimen, e.g., by about 20 weeks from treatment initiation;
A gain in EORTC-QLQ-C30 GHS/QoL score of about 15 when on the q4w regimen, or of about 13 when on the q2w regimen, e.g., by about 24 weeks from treatment initiation;
A gain in EORTC-QLQ-C30 GHS/QoL score of about 14 when on the q4w regimen, or of about 13 when on the q2w regimen, e.g., by about 28 weeks from treatment initiation;
A FACIT-fatigue score of about 45 (±4) e.g., when on the q4w regimen, or a score of 43 (±8) e.g., when on the q2w regimen, for example by week 2 following treatment initiation;
A FACIT-fatigue score of about 40 (±11) e.g., when on the q4w regimen, or a score of 40 (±10) e.g., when on the q2w regimen, for example by week 4 following treatment initiation;
A FACIT-fatigue score of about 43 (±7) e.g., when on the q4w regimen, or a score of 39 (±11) e.g., when on the q2w regimen, for example by week 8 following treatment initiation;
A FACIT-fatigue score of about 44 (±7) e.g., when on the q4w regimen, or a score of 40 (±9) e.g., when on the q2w regimen, for example by week 12 following treatment initiation;
A FACIT-fatigue score of about 44 (±5) e.g., when on the q4w regimen, or a score of 41 (±11) e.g., when on the q2w regimen, for example by week 16 following treatment initiation;
A FACIT-fatigue score of about 43 (±7) e.g., when on the q4w regimen, or a score of 37 (±13) e.g., when on the q2w regimen, for example by week 20 following treatment initiation;
A FACIT-fatigue score of about 44 (±7) e.g., when on the q4w regimen, or a score of 43 (±9) e.g., when on the q2w regimen, for example by week 24 following treatment initiation;
A FACIT-fatigue score of about 44 (±7) e.g., when on the q4w regimen, or a score of 42 (±10) e.g., when on the q2w regimen, for example by week 28 following treatment initiation;
An EORTC-QLQ-C30 physical functioning score of about 94 (±9) e.g., when on the q4w regimen, or a score of 85 (±19) e.g., when on the q2w regimen, for example by week 2 following treatment initiation;
An EORTC-QLQ-C30 physical functioning score of about 90 (±9) e.g., when on the q4w regimen, or a score of 84 (±19) e.g., when on the q2w regimen, for example by week 4 following treatment initiation;
An EORTC-QLQ-C30 physical functioning score of about 93 (±7) e.g., when on the q4w regimen, or a score of 84 (±19) e.g., when on the q2w regimen, for example by week 8 following treatment initiation;
An EORTC-QLQ-C30 physical functioning score of about 91 (±9) e.g., when on the q4w regimen, or a score of 88 (±16) e.g., when on the q2w regimen, for example by week 12 following treatment initiation;
An EORTC-QLQ-C30 physical functioning score of about 90.0 (±9.6) e.g., when on the q4w regimen, or a score of 85.0 (±19.9) e.g., when on the q2w regimen, for example by week 16 following treatment initiation;
An EORTC-QLQ-C30 physical functioning score of about 94 (±8) e.g., when on the q4w regimen, or a score of 82 (±19) e.g., when on the q2w regimen, for example by week 20 following treatment initiation;
An EORTC-QLQ-C30 physical functioning score of about 91 (±9) e.g., when on the q4w regimen, or a score of 86 (±19) e.g., when on the q2w regimen, for example by week 24 following treatment initiation;
An EORTC-QLQ-C30 physical functioning score of about 95 (±6) e.g., when on the q4w regimen, or a score of 91 (±17) e.g., when on the q2w regimen, for example by week 28 following treatment initiation;
An EORTC-QLQ-C30 GHS/QoL score of about 76 (±18) e.g., when on the q4w regimen, or a score of 75 (±17) e.g., when on the q2w regimen, for example by week 2 following treatment initiation;
An EORTC-QLQ-C30 GHS/QoL score of about 71 (±26) e.g., when on the q4w regimen, or a score of 76 (±23) e.g., when on the q2w regimen, for example by week 4 following treatment initiation;
An EORTC-QLQ-C30 GHS/QoL score of about 69 (±21) e.g., when on the q4w regimen, or a score of 70 (±26) e.g., when on the q2w regimen, for example by week 8 following treatment initiation;
An EORTC-QLQ-C30 GHS/QoL score of about 72 (±15) e.g., when on the q4w regimen, or a score of 73 (±20) e.g., when on the q2w regimen, for example by week 12 following treatment initiation;
An EORTC-QLQ-C30 GHS/QoL score of about 72 (±22) e.g., when on the q4w regimen, or a score of 69 (±29) e.g., when on the q2w regimen, for example by week 16 following treatment initiation;
An EORTC-QLQ-C30 GHS/QoL score of about 77 (±20) e.g., when on the q4w regimen, or a score of 67 (±25) e.g., when on the q2w regimen, for example by week 20 following treatment initiation;
An EORTC-QLQ-C30 GHS/QoL score of about 76 (±19.0) e.g., when on the q4w regimen, or a score of 74 (±29) e.g., when on the q2w regimen, for example by week 24 following treatment initiation; AND/OR
An EORTC-QLQ-C30 GHS/QoL score of about 75 (±17) e.g., when on the q4w regimen, or a score of 74 (±24) e.g., when on the q2w regimen, for example by week 28 following treatment initiation.
   Achieving a given score or score improvement (e.g., in EORTC-QLQ-C30 GHS/QoL score; EORTC-QLQ-C30 physical functioning score or FACIT-Fatigue score) includes embodiments wherein the subject's conditions are measured, for example, according to the relevant questionnaire or scale; and embodiments wherein, though not measured with the questionnaire or scale, the subject's condition would achieve such a score or improvement if measured with the questionnaire or scale.

In an embodiment of the disclosure, in spite of treatment with a combination therapy of the present disclosure, a subject may be administered a transfusion with red blood cells (RBCs) for example, according to the following:
Transfuse with RBCs if the hemoglobin level is 59 g/dL with new onset or worsening signs or symptoms resulting from anemia that are of sufficient severity to warrant transfusion; or
Transfuse with RBCs if the hemoglobin level is 57 g/dL with or without signs or symptoms of anemia.

In an embodiment of the disclosure, a subject receiving a combination therapy of the present disclosure receives an “intensified” treatment, e.g., if the subject experiences breakthrough hemolysis that is not due to a complement activating condition (e.g., intercurrent infection) and/or if the subject experiences inadequate LDH response (i.e., LDH >1.5×ULN) that is sustained (e.g., on 2 consecutive measurements spanning at least about 2 weeks). Intensified treatment includes one or more doses of anti-C5 antibody or antigen-binding fragment, preferably Pozelimab and/or C5 iRNA, preferably Cemdisiran, in addition to the doses specified in a combination therapy as discussed herein, for example,
For subjects receiving Pozelimab 400 mg SC Q4W (±3, 4, 5, 6 or 7 days) and Cemdisiran 200 mg SC Q4W (±3, 4, 5, 6 or 7 days), the subject receives a single administration of 30 mg/kg Pozelimab intravenously (IV) on the day of intensification and an intensified Pozelimab regimen of 400 mg Q2W (±3, 4, 5, 6 or 7 days) along with Cemdisiran 200 mg Q4W (±3, 4, 5, 6 or 7 days) starting on the day of intensification.
For subjects receiving Pozelimab 400 mg SC Q2W (±3, 4, 5, 6 or 7 days) and Cemdisiran 200 mg SC Q4W (±3, 4, 5, 6 or 7 days), the subject receives a single administration of 30 mg/kg Pozelimab IV on the day of intensification and a re-initiation of the combination regimen of Pozelimab 400 mg SC Q2W (±3, 4, 5, 6 or 7 days) and Cemdisiran 200 mg SC Q4W (±3, 4, 5, 6 or 7 days) starting on the day of intensification.

In an embodiment of the disclosure, a subject who receives intensified treatment (e.g., who was receiving the Eculizumab switch regimen), preferably for treatment of PNH, receives administration of 30 mg/kg Pozelimab IV on the day of initiation (e.g., which can be initiated from day 57 onward) in addition to a maintenance regimen with a shortened frequency of Pozelimab administration 400 mg SC Q2W (±3, 4, 5, 6 or 7 days) along with Cemdisiran 200 mg SC Q4W (±3, 4, 5, 6 or 7 days) e.g., for a period of 32 weeks starting on the day of initiation.

In some embodiments, the disclosed combination therapy includes administering the anti-C5 antibody or antigen-binding fragment thereof to a subject in need thereof in one or more doses administered about four times a week, twice a week, once a week, once every two weeks, once every three weeks, once every four weeks, once every five weeks, once every six weeks, once every eight weeks, once every twelve weeks, or less frequently so long as a therapeutic response is achieved. In one embodiment, the disclosed anti-C5 antibody or antigen-binding fragment thereof (e.g., Pozelimab) is administered to the subject once every two weeks or once every four weeks.

As used herein, the expression “in combination with” means that the anti-C5 antibody or antigen-binding fragment thereof is administered before, after, or concurrently with the C5 iRNA. This expression includes sequential or concurrent administration of the anti-C5 antibody or antigen-binding fragment thereof and a C5 iRNA.

In some embodiments, when the anti-C5 antibody or antigen-binding fragment thereof is administered “before” the C5 iRNA, the anti-C5 antibody or antigen-binding fragment thereof may be administered more than 12 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 1 week, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the C5 iRNA.

In some embodiments, when the anti-C5 antibody or antigen-binding fragment thereof is administered “after” the C5 iRNA, the anti-C5 antibody or antigen-binding fragment thereof may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, about 1 week, about 2 weeks, about 3 weeks, about 4 weeks, about 5 weeks, about 5 weeks, about 7 weeks, about 8 weeks, about 9 weeks, about 10 weeks, about 11 weeks, about 12 weeks, or more than 12 weeks after the administration of the C5 iRNA.

As used herein, “concurrent” administration means that the anti-C5 antibody or antigen-binding fragment thereof (e.g., Pozelimab) and a C5 iRNA (e.g., Cemdisiran) are administered to the subject in a single dosage form (e.g., co- formulated) or in separate dosage forms administered to the subject during the same treatment episode, preferably within about 1 or 2 hours or 30 minutes or less of each other (i.e., before, after, or at the same time), such as about 15 minutes or less, or about 5 minutes or less. If administered in separate dosage forms, each dosage form may be administered via the same route (e.g., both administered intravenously, subcutaneously, etc.); or, alternatively, each dosage form may be administered via a different route. In any event, administering the components in a single dosage from, in separate dosage forms by the same route, or in separate dosage forms by different routes are all considered “concurrent” administration” for purposes of the present disclosure. In an embodiment of the disclosure, concurrent subcutaneous doses of anti-C5 antibody or antigen-binding fragment and C5 iRNA are administered by injection into separate arms.

As used herein, “sequential” administration means that each dose of a selected therapy is administered to the subject at a different point in time, e.g., on different days separated by a predetermined interval (e.g., hours, days, weeks, or months). For illustrative purposes, sequential administration may include administering an initial dose of the anti-C5 antibody or antigen-binding fragment thereof (or C5 iRNA), followed by one or more secondary doses the C5 iRNA (or anti-C5 antibody or antigen-binding fragment thereof), optionally followed by one or more tertiary doses of the anti-C5 antibody or antigen-binding fragment thereof (or C5 iRNA). For illustrative purposes, sequential administration may include administering to the subject an initial dose of the anti-C5 antibody or antigen-binding fragment thereof (or C5 iRNA), followed by one or more secondary doses of the C5 iRNA (or anti-C5 antibody or antigen-binding fragment thereof), and optionally followed by one or more tertiary doses of the C5 iRNA (or anti-C5 antibody or antigen-binding fragment thereof).

As used herein, “initial” dose, “secondary” dose, and “tertiary” dose refer to the temporal sequence of administration. Thus, the “initial” dose is the dose which is administered at the beginning of the treatment regimen (also referred to as the “baseline dose”); “secondary” doses are administered after the initial dose; and “tertiary” doses are administered after the secondary doses. The initial, secondary, and tertiary doses may all contain the same amount of the selected therapy or may contain different amounts of the selected therapy.

### Treatment and Administration

The co-formulations and/or combination therapy of the present disclosure (e.g., Cemdisiran/Pozelimab) are useful for the treatment or prevention of a disease or disorder or condition associated with C5 that includes the step of administering a therapeutically effective amount of anti-C5 antibody or antigen-binding fragment and a C5 iRNA, preferably in a co-formulation, e.g., by parenteral route, e.g., intramuscular (IM), subcutaneous (SC), intravenous (IV) or intravitreal (IVT) or intraocular injection. Preferably, about 400 mg of the antibody, preferably Pozelimab, is administered every about 2-4 (e.g., 2, 3 or 4) weeks whereas about 200 mg iRNA, preferably Cemdisiran, is administered about every 4 weeks.

In some embodiments, the disclosed co-formulation and/or combination therapy (e.g., Cemdisiran/Pozelimab) can be used for treating or preventing myasthenia gravis (MG), for example, a 100:100 co-formulation. Signs and symptoms of MG include, but are not limited to, weakness of the eye muscles (ocular myasthenia), drooping of one or both eyelids (ptosis), blurred or double vision (diplopia), a change in facial expression, difficulty swallowing, shortness of breath, impaired speech (dysarthria), weakness in the arms, hands, fingers, legs, and/or neck. Sometimes the severe weakness of myasthenia gravis may cause respiratory failure. Thus, the present disclosure includes methods for treating or preventing MG, in a subject in need thereof, including the steps of administering a therapeutically effective amount of co-formulation of the present disclosure to the subject (e.g., by SC, IM or IV injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

In some embodiments, the disclosed co-formulations and/or combination therapy of the present disclosure (e.g., Cemdisiran/Pozelimab) are useful in treating or preventing atypical hemolytic uremic syndrome (aHUS). Signs and symptoms of aHUS include, but are not limited to, platelet activation, hemolysis, systemic thrombotic microangiopathy (formation of blood clots in small blood vessels throughout the body) leading to stroke, heart attack, kidney failure and/or death, end-stage renal disease, permanent renal damage, abdominal pain, confusion, edema, fatigue, nausea/vomiting, diarrhea, and microangiopathic anemia. Thus, the present disclosure includes methods for treating or preventing aHUS, in a subject in need thereof, including the steps of administering a therapeutically effective amount of co-formulation of the present disclosure to the subject (e.g., by SC, IM or IV injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

In some embodiments, the disclosed co-formulation and/or combination therapy (e.g., Cemdisiran/Pozelimab) can be used for treating or preventing paroxysmal nocturnal hemoglobinuria (PNH), for example, a 50:100 co-formulation (Cemdisiran mg/ml:Pozelimab mg/ml). Signs and symptoms of PNH include, but are not limited to, destruction of red blood cells, thrombosis (including deep vein thrombosis, pulmonary embolism), intravascular hemolytic anemia, red discoloration of urine, symptoms of anemia such as tiredness, shortness of breath, and palpitations, abdominal pain and difficulty swallowing. Thus, the present disclosure includes methods for treating or preventing PNH, in a subject in need thereof, including the steps of administering a therapeutically effective amount of co-formulation of the present disclosure to the subject (e.g., by SC, IM or IV injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

In some embodiments, the disclosed co-formulations and/or combination therapy are useful in treating PNH patients (including PNH patients who have transitioned from Pozelimab monotherapy) by, for example, controlling hemolysis without any breakthrough hemolysis events, achieving hemoglobin stabilization, and/or maintaining normalization of LDH for a sustained period of time (e.g., at least 28 weeks). In some embodiments, the disclosed co-formulations and/or combination therapy are useful in treating PNH patients (including PNH patients who have transitioned from Pozelimab monotherapy) by, for example, improving patient fatigue, improving global health status (GHS)/Quality-of-Life (QoL), and/or improving physical functioning as compared to baseline,

In some embodiments, the disclosed co-formulation and/or combination therapy (e.g., Cemdisiran/Pozelimab) can be used for treating or preventing CHAPLE disease (CD55 deficiency with hyperactivation of complement, angiopathic thrombosis and protein-losing enteropathy). CHAPLE disease is characterized by symptoms such as inflammatory bowel disease, protein losing enteropathy (which can be associated with hypoalbuminemia), hypogammaglobulinemia, intestinal lymphangiectasia, and/or thrombotic events. Thus, the present disclosure includes methods for treating or preventing CHAPLE, in a subject in need thereof, including the steps of administering a therapeutically effective amount of co-formulation of the present disclosure to the subject (e.g., by SC, IM or IV injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

In some embodiments, the disclosed co-formulation and/or combination therapy (e.g., Cemdisiran/Pozelimab) can be used for treating or preventing (including reducing or eliminating signs or symptoms thereof, or reducing complement activation associated therewith) a disease or disorder or condition associated with C5 such as a disorder of inappropriate or undesirable complement activation; systemic inflammatory response in post-pump syndrome due to cardiopulmonary bypass or renal bypass; a neurological disorder; a renal disorder; a hemodialysis complication; an inflammatory disorder; inflammation of an autoimmune disease; thermal injury; an immune complex disorder; an autoimmune disease or a proteinuric kidney disease. Thus, the present disclosure includes methods for treating or preventing any of such disorders, in a subject in need thereof, including the steps of administering a therapeutically effective amount of co-formulation of the present disclosure to the subject (e.g., by SC, IM or IV injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

In some embodiments, the disclosed co-formulation and/or combination therapy (e.g., Cemdisiran/Pozelimab) can be used for treating or preventing (including reducing or eliminating signs or symptoms thereof, or reducing complement activation associated therewith) a disease or disorder or condition associated with C5 such as complement activation due to burn; inherited CD59 deficiency; renal ischemia; a post-ischemic reperfusion condition; adult respiratory distress syndrome; Alport's syndrome; Alzheimer's disease; atherosclerosis; bullous pemphigoid; C3 glomerulopathy; capillary leak syndrome; Crohn's disease; diabetes; diabetic nephropathy; epilepsy; glomerulopathy; Guillain-Barre Syndrome; hemolytic anemia; hyperacute allograft rejection; infectious disease; interleukin-2 induced toxicity during IL-2 therapy; lupus nephritis; membranoproliferative glomerulonephritis; membranoproliferative nephritis; mesenteric artery reperfusion after aortic reconstruction; multiple sclerosis; myasthenia gravis; myocardial infarction; neuromyelitis optica; complement activation due to obesity; Parkinson's disease; progressive kidney failure; psoriasis; renal ischemia-reperfusion injury; rheumatoid arthritis; schizophrenia; SLE nephritis; stroke; systemic lupus erythematosus (SLE); traumatic brain injury; vasculitis; xenograft rejection; CHAPLE disease/syndrome (CD55 deficiency with hyperactivation of complement, angiopathic thrombosis and PLE); complement activation due to frostbite; complement activation due to sepsis. Thus, the present disclosure includes methods for treating or preventing any of such condition or disease, in a subject in need thereof, including the steps of administering a therapeutically effective amount of co-formulation of the present disclosure to the subject (e.g., by SC, IM or IV injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

In some embodiments, the disclosed co-formulations and/or combination therapy of the present disclosure (e.g., Cemdisiran/Pozelimab) are useful for treating or preventing (including reducing or eliminating signs or symptoms thereof, or reducing complement activation associated therewith) a disease or disorder or condition associated with C5 such as a lung disease or disorder such as dyspnea, hemoptysis, ARDS, asthma, chronic obstructive pulmonary disease (COPD), emphysema, pulmonary embolisms and infarcts, pneumonia, fibrogenic dust diseases, injury due to inert dusts and minerals (e.g., silicon, coal dust, beryllium, and asbestos), pulmonary fibrosis, an organic dust disease, chemical injury (due to irritant gasses and chemicals, e.g., chlorine, phosgene, sulfur dioxide, hydrogen sulfide, nitrogen dioxide, ammonia, and hydrochloric acid), smoke injury, thermal injury (e.g., burn or freeze), asthma, allergy, bronchoconstriction, hypersensitivity pneumonitis, a parasitic disease, Goodpasture's Syndrome, pulmonary vasculitis, hereditary angioedema, or immune complex-associated inflammation. Thus, the present disclosure includes methods for treating or preventing any of such condition or disease, in a subject in need thereof, including the steps of administering a therapeutically effective amount of co-formulation of the present disclosure to the subject (e.g., by SC, IM or IV injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

In some embodiments, the disclosed co-formulations and/or combination therapy of the present disclosure (e.g., Cemdisiran/Pozelimab) are useful for treating or preventing (including reducing or eliminating signs or symptoms thereof, or reducing complement activation associated therewith) a disease or disorder or condition associated with C5 which is an ocular disease such as age-related macular degeneration (AMD), diabetic macular edema (DME), diabetic retinopathy, ocular angiogenesis (ocular neovascularization affecting choroidal, corneal or retinal tissue), geographic atrophy (GA), uveitis and neuromyelitis optica. The co-formulations of the present disclosure may be used to treat or to ameliorate at least one sign and/or symptom of dry AMD or wet AMD. Thus, the present disclosure includes methods for treating or preventing any of such condition or disease, in a subject in need thereof, including the steps of administering a therapeutically effective amount of co-formulation of the present disclosure to the subject (e.g., parenteral injection; or preferably, by intraocular or intravitreal injection). In an embodiment of the invention, such a therapeutically effective amount is any of the dosing regimens set forth herein (e.g., one or more doses of 400 mg Pozelimab SC and 200 mg Cemdisiran SC (e.g., every 4 weeks)).

“Treat” or “treating” means to administer a co-formulation of the present disclosure (e.g., Cemdisiran/Pozelimab) to a subject having a disease or disorder or condition associated with C5, such that one or more signs or symptoms thereof in the subject are reduced or eliminated, e.g., reducing complement activation associated therewith.

A therapeutically effective dose or amount of anti-C5 antibody and a C5 iRNA, in a co-formulation for treating a disease or disorder or condition associated with C5, is in the range of about 10-800 mg of each, administered once every 1, 2, 3, 4, 5, 6, 7, or 8 weeks.

A subject or patient—used interchangeably herein—refers to a mammal, preferably, a human. In an embodiment of the disclosure, the subject suffers from a disease or disorder or condition associated with C5, such as PNH or MG or aHUS or CHAPLE. In an embodiment of the disclosure, the subject is or was previously receiving a therapeutic agent for treating the disease or disorder (e.g., a complement inhibitor, such as crovalimab; Eculizumab, tesidolumab, mubodina and/or Ravulizumab) before switching to a co-formulation and/or combination therapy of the present disclosure that includes different agents (e.g., Cemdisiran/Pozelimab). In an embodiment of the present disclosure, the subject is treatment “naïve” having never previously received a complement inhibitor or not having recently received a complement inhibitor, e.g., with in 1, 2, 3, 4, 5 or 6 months. In an embodiment of the present disclosure, a subject has been diagnosed with paroxysmal nocturnal hemoglobinuria which has been confirmed by a history of high-sensitivity flow cytometry. In an embodiment of the present disclosure, the subject has a lactate dehydrogenase of at least 1.5×ULN (upper limit of normal). Sahin et al., Pesg PNH diagnosis, follow-up and treatment guidelines. Am J Blood Res 2016; 6(2):19-27. In an embodiment of the present disclosure, the subject or patient does not have any one or more of the following characteristics:
history of bone marrow transplantation or receipt of an organ transplant
body weight <40 kg
any 2 of the following 3 abnormalities:
   a. Peripheral blood absolute neutrophil count (ANC)<500/μL [<0.5×10⁹/L]; or
   b. Peripheral blood platelet count <20,000/μL; or
   c. Peripheral blood reticulocyte count abnormality defined as <20,000/μL or <1%
Hypocellular bone marrow based on a history of reduced age-adjusted bone marrow cellularity and/or bone marrow cellularity 525%
no documented meningococcal vaccination
unable to take antibiotics for meningococcal prophylaxis
any active, ongoing infection or a recent infection requiring ongoing systemic treatment with antibiotics, antivirals, or antifungals within 2 weeks
history of systemic fungal disease or unresolved tuberculosis (TB), or active or latent tuberculosis infection (LTBI)
positive hepatitis B surface antigen or hepatitis C virus RNA
history of human immunodeficiency virus (HIV) infection
SARS-CoV-2 infection
hereditary complement deficiency
history of active, uncontrolled, ongoing systemic autoimmune diseases
history of liver cirrhosis or liver disease with evidence of current, impaired liver function, or ALT or AST (unrelated to PNH) greater than 3×ULN
eGFR of <30 mL/min/1.73 m² (according to Chronic Kidney Disease-Epidemiology Collaboration equation 2009 [CKD-EPI])
anticipated need for major surgery
cancer within the past 5 years, except for adequately treated basal cell skin cancer, squamous cell skin cancer, or in situ cervical cancer
hypersensitivity to Pozelimab, Cemdisiran
documented functional or anatomic asplenia
pregnant or breastfeeding woman
woman of childbearing potential unwilling to practice highly effective contraception
not vaccinated against Streptococcus pneumoniae and/or Haemophilus influenza type B.

In an embodiment of the disclosure, the subject is receiving or has received a blood transfusion.

In an embodiment of the disclosure, a subject receiving a co-formulation of the present disclosure to treat a disease or disorder or condition associated with C5 achieve a reduction in intravascular hemolysis or blood lactate dehydrogenase (LDH) levels and/or a reduction in the receipt of blood transfusions compared to prior to the initiation of treatment.

### Devices

The present disclosure also provides an injection device comprising the co-formulations of the present disclosure (e.g., Cemdisiran/Pozelimab). An injection device is a device that introduces a substance into the body of a patient via a parenteral route, e.g., intramuscular, subcutaneous, intravitreal, intraocular or intravenous. For example, an injection device may be a syringe (e.g., pre-filled or auto-injector) which, for example, includes a cylinder or barrel for holding fluid to be injected (e.g., the co-formulation), a needle for piecing skin and/or blood vessels for injection of the fluid; and a plunger for pushing the fluid out of the cylinder and through the needle bore. In an embodiment of the disclosure, an injection device that comprises a co-formulation is suitable for subcutaneous, intravitreal or intravenous (IV) injection. Such a device includes a co-formulation in a cannula or trocar/needle which may be attached to a tube which may be attached to a bag or reservoir for holding fluid (e.g., saline; or lactated ringer solution comprising NaCl, sodium lactate, KCl, CaCl₂ and optionally including glucose) introduced into the body of the patient through the cannula or trocar/needle.

The co-formulation can, in an embodiment of the disclosure, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line).

In an embodiment of the disclosure, an injection device is an autoinjector; a jet injector or an external infusion pump. A jet injector uses a high-pressure narrow jet of liquid which penetrate the epidermis to introduce a co-formulation to a patient's body. External infusion pumps are medical devices that deliver the co-formulation into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

### Beta-Hexosaminidase (Beta-Hex)

The present disclosure provides methods for reducing the level of beta-hexosaminidase enzymatic activity in a composition, such as a pharmaceutical co-formulation, e.g., which comprises a molecule which is a substrate for the enzyme (e.g., a co-formulation of an antibody or antigen-binding fragment thereof (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; crovalimab; Eculizumab, tesidolumab, mubodina or Ravulizumab; preferably, Pozelimab) isolated from a mammalian host cell and including beta hexosaminidase (beta hex) contaminants and an iRNA that includes a ligand having one or more terminal GalNAc and/or GlcNAc residues which are substrates of beta-hex). Typically, the beta-hexosaminidase is present in small, trace amounts that have carried over with the antibody or antigen-binding fragment after isolation from the host cell and/or host cell growth medium.

Evidence suggests that the amount of beta-hex in antibody compositions is dependent on the particular antibody. Most antibodies tested exhibited <2 ppm beta-hex. Pozelimab has been observed to have somewhat higher beta-hex levels than many other antibodies tested. Purification steps, however, may also affect the degree of antibody-to-antibody beta-hex content variability.

In an embodiment of the disclosure, the beta-hex is Chinese hamster beta-hex. In an embodiment of the disclosure, the beta-hex is characterized as mammalian beta-hex, e.g., mouse or human beta-hex. Beta-hex may, in an embodiment of the disclosure, be fungal, e.g., from yeast such as Candida albicans or Pichia (e.g., Pichia pastoris).

In an embodiment of the disclosure, the antibody or fragment binds specifically to C5, tumor necrosis factor alpha, PD-1, PD-L1, VEGF, VEGF receptor, HER2, CTLA4, Leptin receptor, CD3, CD28, CD20, IL-23 and/or EGFR. In an embodiment of the disclosure, the iRNA binds to a polynucleotide encoding any of such genes.

The present disclosure includes co-formulations that preferably include no detectable beta-hex. However, included within the scope of the present disclosure are co-formulations (e.g., Cemdisiran/Pozelimab) that:
include no more than about 0.170 micrograms/ml beta-hex, e.g., about 0.04; 0.05; 0.06; 0.06; 0.0605; 0.0605; 0.0605; 0.063; 0.07; 0.07; 0.0765; 0.078; 0.08; 0.14; 0.141; 0.15; 0.1525; 0.166; or 0.17; or no more than any of such concentrations;
include no more than about 2, 1.9, 1.65, 1.75, 1, 0.9, 0.8, 0.75, 0.7 or 0.5 parts per million (ppm) molar ratio of beta-hex to antibody;
Include no more than about 1% Cemdisiran Impurity 1 (e.g., about 0.2, 0.4, 0.6, 0.8, 0.9 or 1%);
Exhibit an increase in percentage of Cemdisiran Impurity 1, 2 and 3 over time relative to the total Cemdisiran (Cemdisiran+any Cemdisiran impurities) at initiation of storage characterized by the formula 0.9+3.84 (months of storage)^{1/2} or 0.9+2.84 (months of storage)^{1/2} when stored at 40° C. (e.g., in a formulation includes about 100 mg/ml Pozelimab and about 100 mg/ml Cemdisiran), e.g., wherein impurity percentage is as measured by dIPRP (denaturing ion pair reverse phase chromatography);
Exhibit an increase in percentage of Cemdisiran Impurity 1, 2 and 3 over time relative to the total Cemdisiran (Cemdisiran+any Cemdisiran impurities) at initiation of storage characterized by the formula 0.8+4.79 (months of storage)^{1/2} or 0.9+3.30 (months of storage)^{1/2} when stored at 40° C. (e.g., in a formulation includes about 100 mg/ml Pozelimab and about 50 mg/ml Cemdisiran);
Has about 91% Cemdisiran before storage (at t=0);
Has no less than about 80% Cemdisiran after 1, 1%, 2, 2% or 3 years storage at 2-8° C.;
and/or
Has about 80% to about 91% Cemdisiran.

In humans, lysosomal β-hexosaminidases catalyze the hydrolysis of β-glycosidically linked N-acetylglucosamine (GlcNAc) and N-acetylgalactosamine (GalNAc) residues from the nonreducing end of a number of glycoconjugates (may be referred to herein in terms of a molecule conjugated to a ligand that comprises one or more terminal GlcNAc or GalNAc residues). Three different isoforms of the dimeric enzymes are known: β-hexosaminidase A (HexA), which represents the heterodimer of the noncovalently linked α and β chain, and the homodimeric isoenzymes β-hexosaminidase B (HexB, ββ) and β-hexosaminidase S (HexS, αα). β-hexosaminidases are particularly important for the lysosomal catabolism of glycosphingolipids, essential membrane components of eukaryotic cell surfaces. See Wendeler & Sandhoff, Hexosaminidase assays, Glycoconj J (2009) 26:945-952.

Assays for beta-hexosaminidases are commercially available. See β-Hexosaminidase Activity Assay, Tribioscience (Sunnyvale, CA). For example, a colorometric assay determines the conversion of p-Nitrophenyl N-acetyl-β-D-glucosaminide to and N-acetyl-D glucosamine and p-Nitrophenol which can be measured at absorbance (OD 405 nm).

The optimal pH for beta-hexosaminidase activity against Cemdisiran was measured to be about 6. Thus, such activity can be reduced by changing the pH to a value above or below 6, for example, 6.5. A pH higher than 6 led to greater Cemdisiran stability; however, in a co-formulation, an antibody such as Pozelimab, an increased pH leads to an increase in % Region1/acidic charge species for the antibody. Some co-formulations of the present disclosure have been formulated to reach a balance of conditions that lead to stable Cemdisiran while still maintaining stability in Pozelimab.

The present disclosure includes methods for reducing beta-hexosaminidase activity against a double stranded RNA (dsRNA) substrate (e.g., that includes a terminal GalNAc such as Cemdisiran) in a composition comprising adjusting the pH to a value above or below about 6, for example, to a value that is no closer than 0.5 to 6.

Beta-hexosaminidase activity has also been shown to be reduced in the presence of N-acetylgalactosamine (GalNAc) or N-acetylglucosamine (GlcNAc). The present disclosure includes methods for reducing beta-hexosaminidase activity against a double stranded RNA (dsRNA) substrate (e.g., that includes a terminal GalNAc such as Cemdisiran) in a composition comprising adding N-acetylgalactosamine (GalNAc) or N-acetylglucosamine (GlcNAc) to the composition. For example, about 5% (w/v) GlcNAc and/or GalNAc can be added.

### EXAMPLES

These examples are intended to exemplify the present disclosure are not a limitation thereof. Compositions and methods, including dosing regimens, set forth in the Examples form part of the present disclosure.

### Example 1: Quantitation of Beta-Hexosaminidase Host Cell Protein Impurity by UPLC-SRM-MS/MS

In this Example, an accurate and absolute quantification of the host cell protein, beta-hexosaminidase as a process-related impurity in Pozelimab antibody preparations was performed.

### Materials

Recombinant beta-hexosaminidase protein and antibody drug substance were produced in-house. Tris-HCl, Tris(2-carboxyethyl)phosphine (TCEP HCl), iodoacetamide (IAM), and formic acid (FA) were from Thermo Fisher Scientific (Waltham, MA). Sequencing grade modified trypsin was from Promega (Madison, WI). LC/MS grade acetonitrile with 0.1% FA and water with 0.1% FA were purchased from Fisher Scientific (USA). Milli-Q water used in the experiments was generated.

### Methods

Beta-hexosaminidase (‘beta-hex’) calibration standards were prepared by serially diluting recombinant beta-hex spiked in 5 mg/mL of Pozelimab antibody drug substance which was free of beta-hex. The beta-hex standard concentrations were 3.3, 8.2, 20.5, 51.2, 128, 320, and 800 ppm (in moles). Quality controls were prepared separately at 3.3, 7.5, 173, 588, and 800 ppm (in moles).

20 μL of beta-hex calibration standards, controls and samples were transferred to a 96 well plate and concentrated to dryness with a sample concentrator. Then the dried proteins were reconstituted in the denaturation and reduction solution (8 M urea and 10 mM TCEP-HCl) with brief mixing and incubated at 56° C. with shaking for 30 minutes. After cooling, the mixtures were alkylated in 10 mM of IAM at room temperature in the dark for 30 minutes. After alkylation, 10 μg of trypsin was added to the mixture (1:10 ratio of trypsin to substrate) and incubated at 37° C. with shaking overnight. At the end of digestion, 10% FA was added to quench the reaction.

The digested peptide mixture was injected into liquid chromatography (Agilent 1290 Infinity II LC Systems) coupled with the Agilent 6495B Triple Quadrupole mass spectrometer to perform MRM analysis. The separation was conducted by reversed-phase liquid chromatography using an ACQUITY UPLC BEH130 C18 column (2.1×50 mm, 1.7 μm; Waters). Mobile phase A was 0.1% FA in water, and mobile phase B was 0.1% FA in acetonitrile. The initial gradient started at 3% B for 0.5 min, then increased to 35% B over 10 min, and was followed by 90% B wash for 2.4 min and 3% B equilibration for 2.4 min. A flow rate of 0.4 mL/min was used in the gradient. The Agilent Jet Stream electrospray ionization (AJS ESI source) was applied with heated nitrogen as the sheath gas and drying gas at 400° C. and 180° C., respectively, at a flow rate of 12 L/min. The MS was operated in positive mode with capillary voltage at 3000 V, nozzle voltage at 300 V and nebulizer pressure at 35 psi. Pre-selected mass-to-charge ratio (m/z) of precursor and product ion pair of beta-hex peptides were fragmented with optimized collision energy and detected in the mass spectrometer. Agilent MassHunter Workstation Data Acquisition for 6400 Series Triple Quadrupole, version B.10.0, was used to run the LC/MS system. Agilent MassHunter Quantitative Analysis, version B.09.00, was used for data analysis.

### Assay Performance Summary

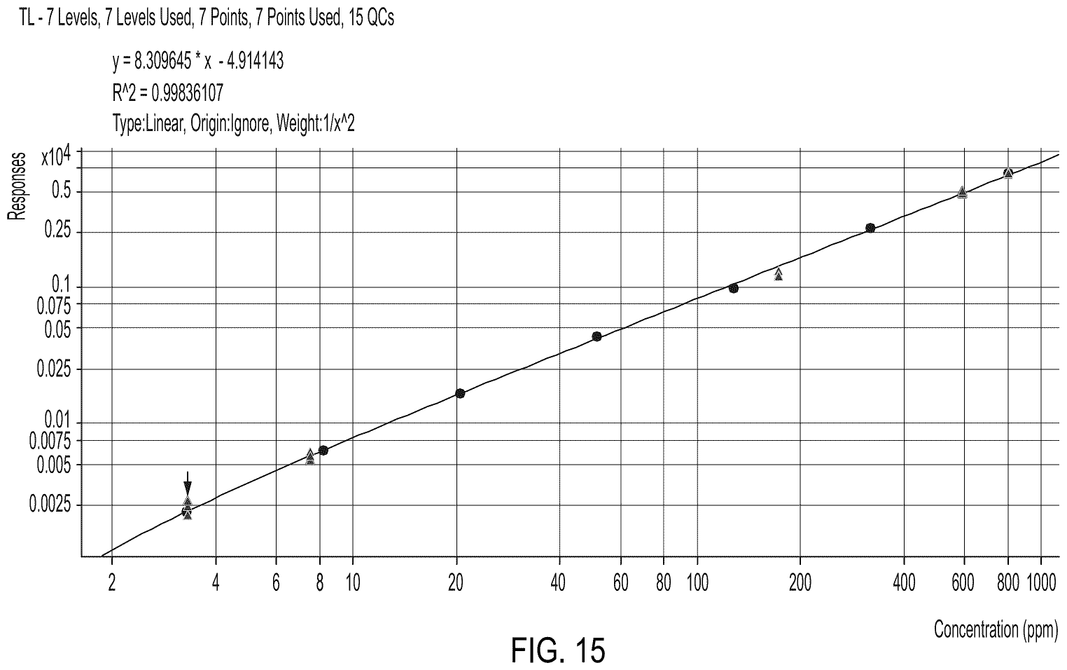

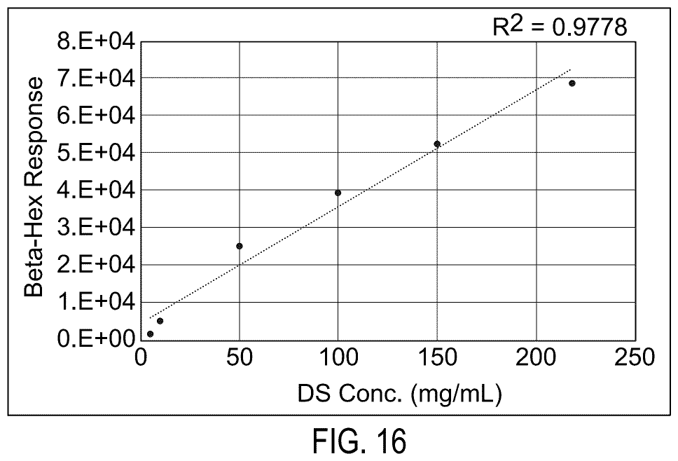

The assay was performed in 5 mg/mL beta-hex free antibody drug substance and ranged from 3.3-800.0 ppm (in moles). Five known concentration samples, LLOQ, LQC, MQC, HQC, and ULOQ, always prepared in triplicate for evaluation during assay development process. Inter-assay accuracy and precision met acceptance criteria of QCs (±25% for LLOQ, ±20% for other QCs) except for one LQC (14/15 of QCs at Day 1) failed at accuracy (Table 1-2). Intra-assay accuracy and precision were calculated from the triplicate QC analysis prepared from single set experiment performed at Day 2 instead of duplicate preparations. Both intra-assay accuracy and precision met acceptance criteria of all QCs (Table 1-3). Mass to charge ratio of quantifier target peptide (‘TLDAMAFNK’; m/z transition: 505.9 >796.7) was specific to beta-hex in assay matrix and other components that might be present in digestion buffer. Dilution linearity evaluated using spiked standards showed that recovery of Beta-hex at 5, 10, 50, 100, 150 and 218 mg/mL antibody matrix was linear (FIG. 16). The assay calibration curve ranged from 3.3-800.0 ppm with 1/x2 weighting was developed to construct beta-hex relative response-standard concentrations relationship (FIG. 15). Interference from Cemdisiran up to 5 mg/mL (1:1 mAb:Cemdisiran ratio) spiked in four QCs (LQC, MQC, HQC, and ULOQ) were evaluated and 92% (11/12) of QCs met acceptance criteria (Table 1-4). Instrument reproducibility test showed that instrument injection and detector were reproducible over repeated sample injections (Table 1-5). Freeze-thaw stability test using QCs showed that samples were stable after 3 freeze-thaw cycles (Table 1-6). Processed sample was also shown to be stable in autosampler at 4° C. up to 48 hours (Table 1-7). In summary, all parameters evaluated met the acceptance criteria (Table 1-1).

### Beta-Hex Measurement for Pozelimab Lots

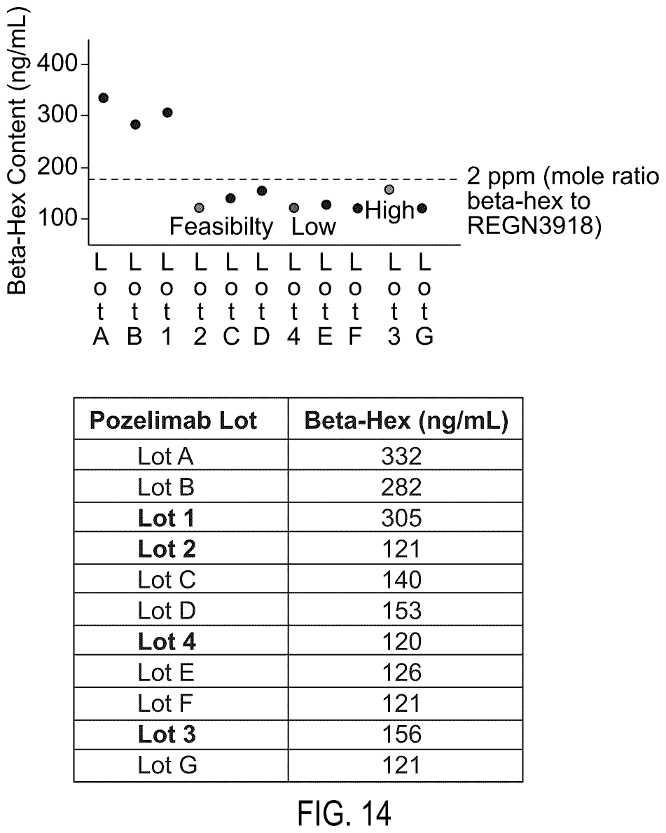

Eleven lots of Pozelimab were analyzed for beta-hex levels by the SRM-LC-MS/MS assay (see method section). The abundance of beta-hex in Pozelimab Lot A, Lot B and Lot 1 (282-332 ng/mL) were at least 2-times higher than in the Lot 2, Lot C, Lot D, Lot 4, Lot E, Lot F, Lot 3 and Lot G materials (120-156 ng/mL). See beta-hex levels measured in various lots in FIG. 14.

TABLE 1-1
Assay Performance Summary

| Evaluation Parameters and Acceptance Criteria | Result |
|---|---|
| Assay Range | |
| 5 mg/mL mAb matrix (ULOQ-LLOQ) | 800.0-3.3 ppm (moles) |
| Inter assay-Accuracy | |
| % Accuracy 80-120% (LLOQ 75-125%) | 84-114% (one LQC failed; |
| | overall 93% of QCs passed) |
| Inter assay-Precision | |
| CV % ≤ 20% (LLOQ ≤ 25%) | 2.3-14.5% |
| Intra assay-Accuracy | |
| % Accuracy 80-120% (LLOQ 75-125%) | 84-113% |
| Intra assay-Precision | |
| CV % ≤ 20% (LLOQ ≤ 25%) | 1.6-10.7% |
| Selectivity | |
| Blank Assay Matrix | BLQ |
| Blank Digest Buffer | BLQ |
| Cemdisiran (5 mg/mL) | BLQ |
| Interference (up to 5 mg/mL Cemdisiran; | |
| mAb:Cemdisiran ratio = 1:1) | |
| % Accuracy 80-120% (LQC, MQC, HQC, ULOQ) | 98-112% (one ULOQ failed, |
| CV % ≤ 20% (LQC, MQC, HQC, ULOQ) | overall 92% of QCs passed) |
| | 0.1-8.7% |
| Carryover | |
| Blank Buffer After ULOQ | BLQ |
| Instrument Reproducibility (Injector and Detector) | |
| % Accuracy 80-120% (LLOQ 75-125%) | 85-112% |
| CV % ≤ 20% (LLOQ ≤ 25%) | 1.2-10.7% |
| Processed Sample Stability (48 hrs) | |
| % Accuracy 80-120% (LLOQ 75-125%) | 84-118% |
| CV % ≤ 20% (LLOQ ≤ 25%) | 3.5-11.0% |
| Sample Freeze-Thaw Stability (3-cycle) | |
| % Accuracy 80-120% (LLOQ 75-125%) | 83-105% |
| CV % ≤ 20% (LLOQ ≤ 25%) | 1.9-11.5% |

TABLE 1-2
Inter-assay Accuracy and Precision

| | | |
|---|---|---|
| QC | Accuracy (%) | CV |

| (N = 3) | Day 1 | Day 2 | (%) |
|---|---|---|---|
| LLOQ | 97 | 84 | 7.7 |
| LLOQ | 97 | 87 | |
| LLOQ | 100 | 102 | |
| LQC | 91 | 105 | 14.5 |
| LQC | 68 | 102 | |
| LQC | 93 | 100 | |
| MQC | 102 | 99 | 3.0 |
| MQC | 94 | 96 | |
| MQC | 97 | 97 | |
| HQC | 107 | 89 | 6.8 |
| HQC | 95 | 96 | |
| HQC | 92 | 91 | |
| ULOQ | 110 | 107 | 2.3 |
| ULOQ | 109 | 113 | |
| ULOQ | 114 | 110 | |

TABLE 1-3
Intra-assay Accuracy and Precision

| QC | Accuracy | CV |
|---|---|---|
| (N = 3) | (%) | (%) |
| LLOQ | 84 | 10.7 |
| LLOQ | 87 | |
| LLOQ | 102 | |
| LQC | 105 | 2.8 |
| LQC | 102 | |
| LQC | 100 | |
| MQC | 99 | 1.6 |
| MQC | 96 | |
| MQC | 97 | |
| HQC | 89 | 4.2 |
| HQC | 96 | |
| HQC | 91 | |
| ULOQ | 107 | 2.6 |
| ULOQ | 113 | |
| ULOQ | 110 | |

TABLE 1-4
Instrument Reproducibility (injector and detector)

| | Accuracy | CV |
|---|---|---|
| QC | (%) | (%) |
| LLOQ - injection1 | 102 | 3.1 |
| LLOQ - injection2 | 101 | |
| LLOQ - injection3 | 96 | |
| LQC - injection1 | 100 | 10.7 |
| LQC - injection2 | 106 | |
| LQC - injection3 | 85 | |
| MQC - injection1 | 97 | 2.8 |
| MQC - injection2 | 92 | |
| MQC - injection3 | 96 | |
| HQC - injection1 | 91 | 1.8 |
| HQC - injection2 | 91 | |
| HQC - injection3 | 94 | |
| ULOQ - injection1 | 110 | 1.2 |
| ULOQ - injection2 | 112 | |
| ULOQ - injection3 | 110 | |

TABLE 1-5
Interference: (1:1 mAb:Cemdisiran ratio)

| | Accuracy (%) | | |
|---|---|---|---|
| QC | 5 mg/mL | 5 mg/mL mAb + | CV |
| (N = 3) | mAb | 5 mg/mL Cemdisiran | (%) |
| LQC | 101 | 98 | 6.5 |
| LQC | 116 | 105 | |
| LQC | 107 | 101 | |
| MQC | 110 | 110 | 1.0 |
| MQC | 111 | 110 | |
| MQC | 109 | 108 | |
| HQC | 111 | 108 | 4.9 |
| HQC | 115 | 102 | |
| HQC | 116 | 108 | |
| ULOQ | 114 | 111 | 3.8 |
| ULOQ | 115 | 112 | |
| ULOQ | 117 | 123 | |

TABLE 1-6
Processed Sample Stability (48 hrs)

| QC | Accuracy | CV |
|---|---|---|
| (N = 3) | (%) | (%) |
| LLOQ | 84 | 11.0 |
| LLOQ | 87 | |
| LLOQ | 102 | |
| LLOQ-48 | 110 | |
| LLOQ-48 | 109 | |
| LLOQ-48 | 102 | |
| LQC | 105 | 6.5 |
| LQC | 102 | |
| LQC | 100 | |
| LQC-48 | 92 | |
| LQC-48 | 105 | |
| LQC-48 | 91 | |
| MQC | 99 | 3.5 |
| MQC | 96 | |
| MQC | 97 | |
| MQC-48 | 104 | |
| MQC-48 | 103 | |
| MQC-48 | 102 | |
| HQC | 89 | 5.2 |
| HQC | 96 | |
| HQC | 91 | |
| HQC-48 | 100 | |
| HQC-48 | 102 | |
| HQC-48 | 97 | |
| ULOQ | 107 | 3.7 |
| ULOQ | 113 | |
| ULOQ | 110 | |
| ULOQ-48 | 116 | |
| ULOQ-48 | 117 | |
| ULOQ-48 | 118 | |

TABLE 1-7
Freeze-thaw Stability (3 cycles)

| | Accuracy (%) | | |
|---|---|---|---|
| | Fresh | 3x FT | CV |
| QC | (N = 3) | (N = 1) | (%) |
| LLOQ | 83 | 85 | 11.5 |
| LLOQ | 102 | | |
| LLOQ | 102 | | |
| LQC | 89 | 90 | 7.6 |
| LQC | 105 | | |
| LQC | 93 | | |
| MQC | 97 | 92 | 3.0 |
| MQC | 99 | | |
| MQC | 96 | | |
| HQC | 100 | 99 | 1.9 |
| HQC | 96 | | |
| HQC | 99 | | |
| ULOQ | 101 | 97 | 3.2 |
| ULOQ | 103 | | |
| ULOQ | 97 | | |

### Example 2: Cemdisiran+ Pozelimab Stability Study

Research studies were initiated to assess the long-term, accelerated, and stress stability for the co-formulation of Cemdisiran and Pozelimab in a single formulation. Co-formulations were manufactured and Cemdisiran was reconstituted in WFI (water for injection).

Glass vials were filled with filtered co-formulation (100:100 or 75:150 Cemdisiran mg/mL: Pozelimab (mg/mL)). The 100:100 co-formulation contains 100 mg/mL Cemdisiran, 100 mg/mL Pozelimab, 10 mM histidine, 50 mM arginine hydrochloride, 0.075% (w/v) polysorbate 80, 1% (w/v) sucrose, pH 6.0. The 75:150 co-formulation contains 75 mg/mL Cemdisiran, 150 mg/mL Pozelimab, 15 mM histidine, 75 mM arginine hydrochloride, 0.1125% (w/v) polysorbate 80, 1.5% (w/v) sucrose, pH 6.0.

A Cemdisiran only liquid formulation was also manufactured (100 mg/mL Cemdisiran in 50 mM Arg HCl, 10 mM His, 1% sucrose, 0.075% PS80, pH 6) and used as a control for this study.

Long-term, Accelerated, and Stress Stability Study. An outline of the long-term, accelerated, and stress stability conditions is presented in Table 2-1.

TABLE 2-1
Research Stability Studies for Cemdisiran +
Pozelimab Initial Feasibility Assessment

| Long-Term | | Container/ |
|---|---|---|
| Stability^{a} | | Closure |
| Storage Temperature | Length of | glass vial |
| 5° C. | Storage (months) | with elastomeric |
| | 0, 1, 3, 6, 9, 12, | stopper |
| | 18, 24, and 36 | |
| Accelerated and Stress Stability | | |
| Incubation Temperature | Length of Incubation | |
| 25° C./60% RH | 0, 1, 3, and 6 months | |
| 40° C./75% RH | 0, 0.5, 1, and 3 months | |
| Stability to Freeze-thaw and Agitation | | |
| Condition | Duration of Stress | |
| Freeze-thaw | 0 and 4 cycles | |
| (−30° C. to RT) | | |
| Agitation | 0 and 48 hours | |
| (250 rpm on orbital shaker) | | |

Results (Pozelimab: 24 month storage at 5° C.). No appreciable change in the physical or chemical stability of Pozelimab in co-formulations was observed in any of the monitored attributes after 24 months at 5° C. (see Table 2-2 and Table 2-3).

Results (Pozelimab: 3-6 month storage at 25° C. or 40° C.). Appreciable changes in the Pozelimab stability (e.g., formation of HMW and charge variants) in a liquid formulation after 25° C./60% RH and 40° C./75% RH storage (see Table 2-4 and Table 2-5) was similar to the Pozelimab only liquid formulation (data not shown).

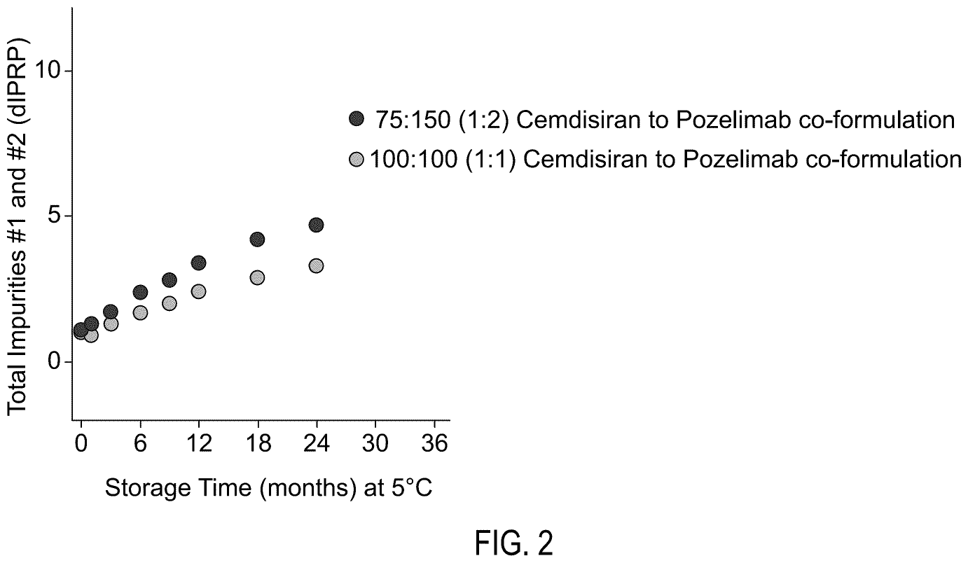

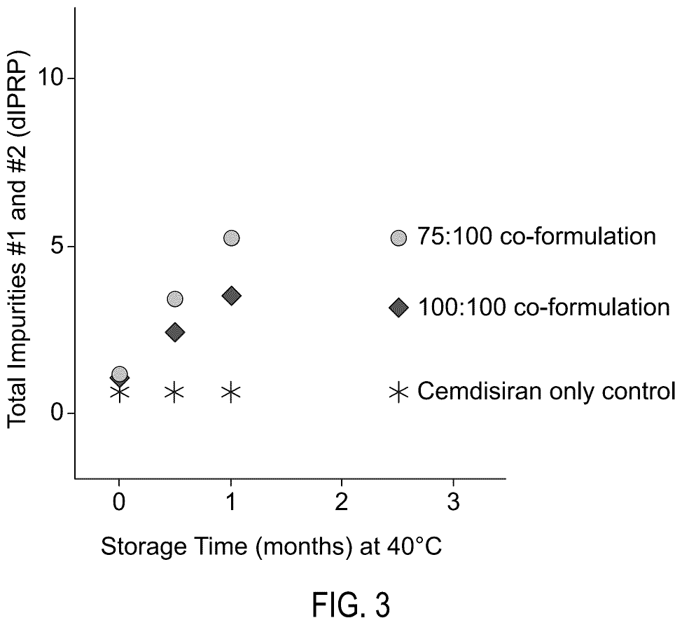

Results (Cemdisiran: Storage at 5° C., 25° C. or 40° C.). There was a decrease in Cemdisiran purity (by dIPRP) in both co-formulations (100:100 and 75:100) at 5° C. and 25° C./60% RH and 40° C./75% RH (see Table 2-2 through Table 2-5, FIG. 2 and FIG. 3). However, no appreciable change in the physical or chemical stability of Cemdisiran control formulation was observed in any of the monitored attributes after 3 months at 40° C./75% RH (Table 2-6 and FIG. 3).

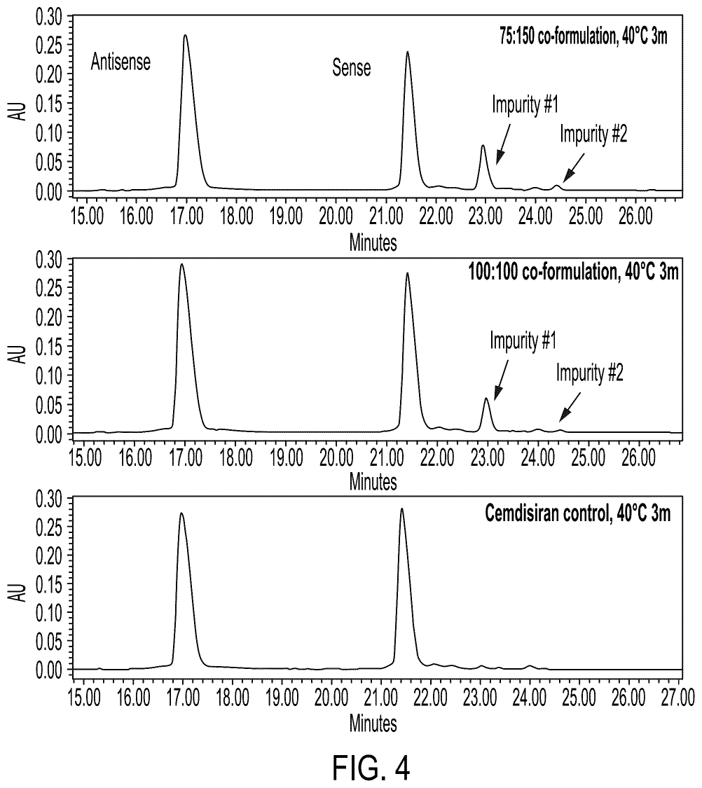

Chromatograms from dIPRP analysis of 40° C./75% RH 3 month samples are shown in FIG. 4. There was a decrease in purity due to a decrease in the sense peak and an increase in two impurities (#1 and #2).

TABLE 2-2
Stability of 100 mg/mL Cemdisiran & 100 mg/mL Pozelimab
Liquid Drug Product After 5° C. Storage (Lot 2)

| | | 100 mg/mL Cemdisiran, 100 mg/mL Pozelimab, 10 mM | | | | | | | | |
|---|---|---|---|---|---|---|---|---|---|---|
| | | histidine, 50 mM arginine hydrochloride, 0.075% (w/v) | | | | | | | | |
| | | polysorbate 80, 1% (w/v) sucrose, pH 6.0 | | | | | | | | |
| Formulation | | Length of Storage (months) | | | | | | | | |
| Assay | | t = 0 | 1 | 3 | 6 | 9 | 12 | 18 | 24 | 36 |
| Color and appearance^{a} | | Pass | Pass | Pass | Pass | Pass | Pass | Pass | Pass | Pass |
| pH | | 6.0 | 6.0 | 6.0 | 6.0 | 6.0 | 6.0 | 6.0 | 6.0 | 6.0 |
| Particulate | ≥10 μm | 38 | 14 | 41 | 165 | 35 | 22 | NR | 16 | 16 |
| Analysis by MFI^{b} | ≥25 μm | 8 | 3 | 24 | 17 | 19 | 5 | NR | 3 | 5 |
| (#/mL) | | | | | | | | | | |
| Cemdisiran Purity (%) by | | 90.5 | 91.1 | 90.8 | 90.0 | 88.8 | 88.7 | 89.0 | 89.4 | 87.4 |
| dIPRP | | | | | | | | | | |
| Total Impurities 1, 2, and 3 | | 1.0 | 0.9 | 1.4 | 1.7 | 2.0 | 2.4 | 2.9 | 3.3 | 4.8 |
| (%) by dIPRP^{c} | | | | | | | | | | |
| Pozelimab | HMW | 1.0 | 0.9 | 0.9 | 1.1 | 1.2 | 1.4 | 1.4 | 1.3 | 1.6 |
| Purity by SE- | Main | 98.3 | 98.4 | 98.4 | 98.0 | 98.4 | 98.0 | 97.8 | 98.2 | 97.4 |
| FLR-UPLC (%) | peak | | | | | | | | | |
| | LMW | 0.7 | 0.7 | 0.7 | 0.9 | 0.4 | 0.7 | 0.8 | 0.6 | 0.9 |
| Pozelimab | Region 1 | 28.6 | 28.2 | 28.6 | 29.9 | 30.1 | 30.8 | 32.0 | 29.0 | 31.0 |
| Charge Variant | Region 2 | 56.2 | 57.2 | 55.7 | 57.1 | 57.8 | 57.3 | 55.7 | 58.1 | 56.6 |
| Analysis by | Region 3 | 15.2 | 14.6 | 15.7 | 13.0 | 12.1 | 11.8 | 12.3 | 12.9 | 12.5 |
| iCIEF^{d} ^{(%)} | | | | | | | | | | |

| | | | | | | | | | |
|---|---|---|---|---|---|---|---|---|---|
| Pozelimab Relative Potency | 101 | 103 | 105 | NR | NR | 85 | NR | 105 | 118 |
| (%) | | | | | | | | | |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak. dIPRP, denaturing ion pair reverse phase; DP, drug product; FDS, Formulated Drug Substance; FLR,
fluorescence; HMW, high molecular weight; iCIEF, imaging capillary isoelectric focusing; LMW, low molecular weight; Micro-Flow Imaging; NR, not required; SE, size exclusion; UPLC, ultra-performance liquid chromatography

TABLE 2-3
Stability of 75 mg/mL Cemdisiran & 150 mg/mL Pozelimab
Liquid Drug Product After 5° C. Storage (Lot 2)

| | | 75 mg/mL Cemdisiran, 150 mg/mL Pozelimab, 15 mM | | | | | | | | |
|---|---|---|---|---|---|---|---|---|---|---|
| | | histidine, 75 mM arginine hydrochloride, 0.1125% (w/v) | | | | | | | | |
| | | polysorbate 80, 1.5% (w/v) sucrose, pH 6.0 | | | | | | | | |
| Formulation | | Length of Storage (months) | | | | | | | | |
| Assay | | t = 0 | 1 | 3 | 6 | 9 | 12 | 18 | 24 | 36 |
| Color and appearance^{a} | | Pass | Pass | Pass | Pass | Pass | Pass | Pass | Pass | Pass |
| pH | | 6.0 | 6.0 | 6.0 | 6.0 | 6.0 | 6.0 | 5.9 | 6.0 | 5.9 |
| Particulate | ≥10 μm | 19 | 19 | 63 | 76 | 8 | 5 | NR | 19 | 5 |
| Analysis by MFI^{b} | ≥25 μm | 5 | 5 | 27 | 17 | 0 | 3 | NR | 5 | 0 |
| (#/mL) | | | | | | | | | | |
| Cemdisiran Purity (%) by | | 90.8 | 90.6 | 90.5 | 89.4 | 88.3 | 87.8 | 87.8 | 87.4 | 85.4 |
| dIPRP | | | | | | | | | | |
| Total Impurities 1, 2, and 3 | | 1.1 | 1.3 | 1.7 | 2.4 | 2.8 | 3.4 | 4.2 | 4.7 | 6.6 |
| (%) by dIPRP^{c} | | | | | | | | | | |
| Pozelimab | HMW | 0.9 | 0.9 | 1.0 | 1.0 | 1.2 | 1.3 | 1.2 | 1.2 | 1.5 |
| Purity by SE- | Main | 98.4 | 98.4 | 98.3 | 98.3 | 98.3 | 98.1 | 98.2 | 98.2 | 97.7 |
| FLR-UPLC (%) | peak | | | | | | | | | |
| | LMW | 0.7 | 0.7 | 0.7 | 0.7 | 0.5 | 0.6 | 0.6 | 0.6 | 0.8 |
| Pozelimab | Region 1 | 28.1 | 28.7 | 28.8 | 30.2 | 31.1 | 30.5 | 32.1 | 30.1 | 30.2 |
| Charge Variant | Region 2 | 56.3 | 55.7 | 55.9 | 57.6 | 57.0 | 57.1 | 53.7 | 55.1 | 55.0 |
| Analysis by | Region 3 | 15.5 | 15.6 | 15.3 | 12.2 | 11.9 | 12.4 | 14.2 | 14.8 | 14.8 |
| iCIEF^{d} (%) | | | | | | | | | | |

| | | | | | | | | | |
|---|---|---|---|---|---|---|---|---|---|
| Pozelimab Relative Potency | 90 | 103 | 114 | NR | NR | 99 | NR | 92 | 93 |
| (%) | | | | | | | | | |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak. dIPRP, denaturing ion pair reverse phase; DP, drug product; FDS, Formulated Drug Substance; FLR, fluorescence; HMW, high molecular weight; iCIEF, imaging capillary isoelectric focusing; LMW, low molecular weight; Micro-Flow Imaging; NR, not required; SE, size exclusion; UPLC, ultra-performance liquid chromatography

TABLE 2-4
Stability of 100 mg/mL Cemdisiran & 100 mg/mL Pozelimab Liquid Drug Product
After 25° C./60% RH and 40° C./75% RH Storage (Lot 2)

| | | 100 mg/mL Cemdisiran, 100 mg/mL Pozelimab, | | | | | | |
|---|---|---|---|---|---|---|---|---|
| | | 10 mM histidine, 50 mM arginine hydrochloride, | | | | | | |
| | | 0.075% (w/v) polysorbate 80, 1% (w/v) sucrose, | | | | | | |
| | | pH 6.0 | | | | | | |
| | | Length of Storage (months) | | | | | | |
| Formulation | | | 25° C./60% RH | | | 40° C./75% RH | | |
| Assay | | t = 0 | 1 | 3 | 6 | 0.5 | 1 | 3 |
| Color and appearance^{a} | | Pass | Pass | Pass | Pass | Pass | Pass | Pass |
| pH | | 6.0 | 6.0 | 6.0 | 6.0 | 6.0 | 6.0 | 6.0 |
| Particulate | ≥10 μm | 38 | 14 | 24 | 28 | 30 | 52 | 22 |
| Analysis by | ≥25 μm | 8 | 3 | 16 | 6 | 14 | 11 | 11 |
| MFI^{b} (#/mL) | | | | | | | | |
| Cemdisiran Purity (%) by | | 90.5 | 90.2 | 87.8 | 85.1 | 90.0 | 88.9 | 85.8 |
| dIPRP | | | | | | | | |
| Total Impurities 1, 2, and 3 | | 1.0 | 2.2 | 4.2 | 6.1 | 2.4 | 3.5 | 6.2 |
| (%) by dIPRP^{c} | | | | | | | | |
| Pozelimab | HMW | 1.0 | 1.2 | 1.4 | 1.8 | 2.0 | 3.0 | 8.5 |
| Purity by SE- | Main peak | 98.3 | 98.1 | 97.9 | 97.3 | 97.2 | 96.1 | 90.2 |
| FLR-UPLC (%) | LMW | 0.7 | 0.7 | 0.7 | 0.9 | 0.7 | 0.9 | 1.3 |
| Pozelimab | Region 1 | 28.6 | 29.1 | 34.4 | 39.7 | 32.8 | 40.4 | 64.5 |
| Charge Variant | Region 2 | 56.2 | 53.5 | 51.7 | 46.6 | 48.4 | 43.0 | 25.5 |
| Analysis by | Region 3 | 15.2 | 17.4 | 13.9 | 13.7 | 18.8 | 16.6 | 10.0 |
| iCIEF^{d} (%) | | | | | | | | |

| | | | | | | | |
|---|---|---|---|---|---|---|---|
| Pozelimab Relative Potency | 101 | NR | NR | NR | NR | 91 | 85 |
| (%) | | | | | | | |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak. dIPRP, denaturing ion pair reverse phase; FLR, fluorescence; HMW, high molecular weight; iCIEF, imaging capillary isoelectric focusing; LMW, low molecular weight; Micro-Flow Imaging; NR, not required; SE, size exclusion; UPLC, ultra-performance liquid chromatography

TABLE 2-5
Stability of 75 mg/mL Cemdisiran & 150 mg/mL Pozelimab Liquid Drug Product
After 25° C./60% RH and 40° C./75% RH Storage (Lot 2)

| | | 75 mg/mL Cemdisiran, 150 mg/mL Pozelimab, | | | | | | |
|---|---|---|---|---|---|---|---|---|
| | | 15 mM histidine, 75 mM arginine hydrochloride, | | | | | | |
| | | 0.1125% (w/v) polysorbate 80, 1.5% (w/v) | | | | | | |
| | | sucrose, pH 6.0 | | | | | | |
| | | Length of Storage (months) | | | | | | |
| Formulation | | | 25° C./60% RH | | | 40° C./75% RH | | |
| Assay | | t = 0 | 1 | 3 | 6 | 0.5 | 1 | 3 |
| Color and appearance^{a} | | Pass | Pass | Pass | Pass | Pass | Pass | Pass |
| Turbidity (Increase in OD at | | 0.00 | 0.00 | 0.00 | 0.00 | 0.00 | 0.00 | 0.01 |
| 405 nm) | | | | | | | | |
| pH | | 6.0 | 6.0 | 6.0 | 6.0 | 6.0 | 6.0 | 6.0 |
| Particulate | ≥10 μm | 19 | 11 | 0 | 33 | 31 | 8 | 8 |
| Analysis by MFI^{b} | ≥25 μm | 5 | 0 | 0 | 5 | 0 | 0 | 3 |
| (#/mL) | | | | | | | | |
| Cemdisiran Purity (%) by | | 90.8 | 88.8 | 85.9 | 82.3 | 88.9 | 87.3 | 82.3 |
| dIPRP | | | | | | | | |
| Total Impurities 1, 2, and 3 | | 1.1 | 3.1 | 6.0 | 8.8 | 3.4 | 5.2 | 9.7 |
| (%) by dIPRP^{c} | | | | | | | | |
| Total Pozelimab Recovered | | 100 | 99 | 99 | 99 | 99 | 98 | 94 |
| (%) | | | | | | | | |
| Pozelimab Purity | HMW | 0.9 | 1.2 | 1.4 | 1.8 | 2.0 | 3.1 | 9.1 |
| by SE-FLR-UPLC | Main | 98.4 | 98.1 | 97.8 | 97.5 | 97.2 | 95.9 | 89.5 |
| (%) | peak | | | | | | | |
| | LMW | 0.7 | 0.7 | 0.8 | 0.8 | 0.8 | 1.0 | 1.4 |
| Pozelimab Charge | Region 1 | 28.1 | 28.2 | 33.5 | 38.7 | 32.1 | 40.5 | 63.3 |
| Variant Analysis | Region 2 | 56.3 | 54.0 | 50.5 | 45.2 | 48.9 | 40.7 | 25.0 |
| by iCIEF^{c} (%) | Region 3 | 15.5 | 17.9 | 16.0 | 16.1 | 19.0 | 18.9 | 11.7 |

| | | | | | | | |
|---|---|---|---|---|---|---|---|
| Pozelimab Relative Potency | 90 | NR | NR | NR | NR | 103 | 104 |
| (%) | | | | | | | |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak. dIPRP, denaturing ion pair reverse phase; FLR, fluorescence; HMW, high molecular weight; iCIEF, imaging capillary isoelectric focusing; LMW, low molecular weight; Micro-Flow Imaging; NR, not required; SE, size exclusion; UPLC, ultra-performance liquid chromatography

TABLE 2-6
Stability of 100 mg/mL Cemdisiran Liquid
Drug Product After 40° C./75% RH Storage

| | |
|---|---|
| Formulation | 100 mg/mL Cemdisiran, 10 mM histidine, 50 |
| | mM arginine hydrochloride, 0.075% (w/v) |
| | polysorbate 80, 1% (w/v) sucrose, pH 6.0 |

| | | Length of Storage (months) | | | |
|---|---|---|---|---|---|
| | | | 40° C./75% RH | | |
| Assay | | t = 0 | 0.5 | 1 | 3 |
| Color and appearance^{a} | | Pass | Pass | Pass | Pass |
| pH | | 6.1 | 6.2 | 6.1 | 6.1 |
| Particulate Analysis by | ≥10 μm | 22 | 5 | 5 | 106 |
| MFI^{b} (#/mL) | ≥25 μm | 5 | 3 | 5 | 27 |

| | | | | |
|---|---|---|---|---|
| Cemdisiran Purity (%) by dIPRP | 91.3 | 91.2 | 91.9 | 91.3 |
| Total Impurities 1, 2, and | 0.6 | 0.6 | 0.6 | 0.7 |
| 3 (%) by dIPRP^{c} | | | | |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs). dIPRP, denaturing ion pair reverse phase; DP, drug product; NR, not required; UPLC, ultra-performance liquid chromatography

### Identifying Cemdisiran Impurities

DIPRP was coupled with MS (mass spectroscopy) to identity the Cemdisiran impurities in co-formulations.

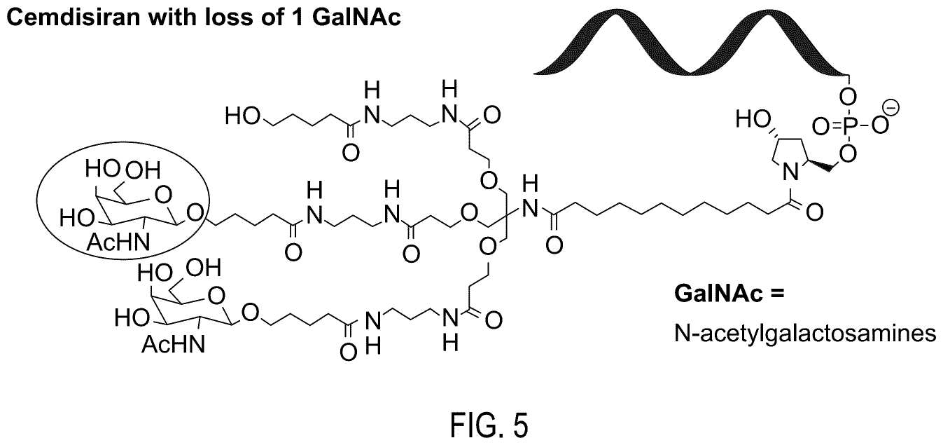

Results confirmed that impurity #1 and #2 in FIG. 5 represented Cemdisiran with the loss of 1 or 2 N-acetylgalactosamines (GalNAc). Three GalNAc at the terminal end of the ligand which is attached to the 3′ end of the sense strand of the Cemdisiran molecule. This ligand (with GalNAcs) enables the molecule to bind to liver receptors and enter liver cells.

Confirmation that Beta-Hexosaminidase is the Host Cell Protein (HCP) in Pozelimab Formulated Drug Substance is Responsible for Cemdisiran Degradation in Co-Formulations

Experiment 1. Co-formulations (100:100 and 75:150) were manufactured as set forth above using Pozelimab from lot 1. A quantitative method was developed by selected reaction monitoring (SRM)-LC-MS/MS, so that there was 2.5x higher beta-hexosaminidase concentration in lot 1 compared to lot 2 material.

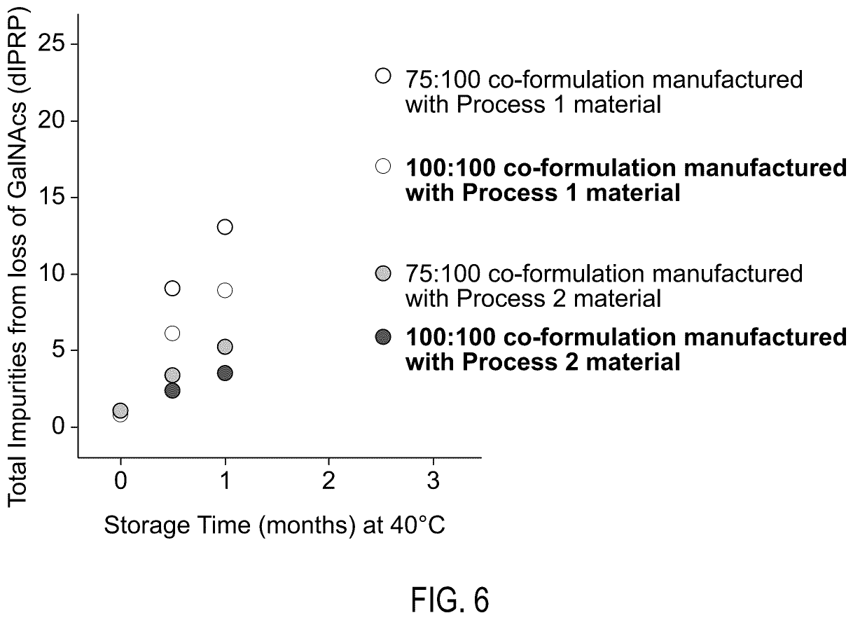

Co-formulations manufactured with the lot 1 material were placed on stability at 5° C., 25° C./60% RH, and 40° C./75% RH. Cemdisiran purity (by dIPRP) for co-formulation manufactured from Pozelimab lot 1 and lot 2 at 40° C./75% are shown in FIG. 6. The rate of Cemdisiran degradation (i.e., the formation of impurities with the loss of 1 or more GalNAc) is 2.3 and 2.8 times faster in co-formulations manufactured with lot 1 material vs lot 2 material. The higher rate of Cemdisiran degradation was similar to the fold increase in beta-hexosaminidase concentration.

TABLE 2-7
Evaluation of Beta-hexosaminidase Levels in Pozelimab Formulated
Drug Substance used to Manufacture Co-formulations

| | Beta-hexosaminidase concentration | | Beta-hexosaminidase | |
|---|---|---|---|---|
| | (ppm mole ratio of | | concentration | |
| | beta-hex to Pozelimab) | | (ng/mL) | |
| Process | Mean | SD | Mean | SD |
| 1 | 3.5 | 0.3 | 306 | 25 |
| 2 | 1.5 | 0.2 | 132 | 15 |

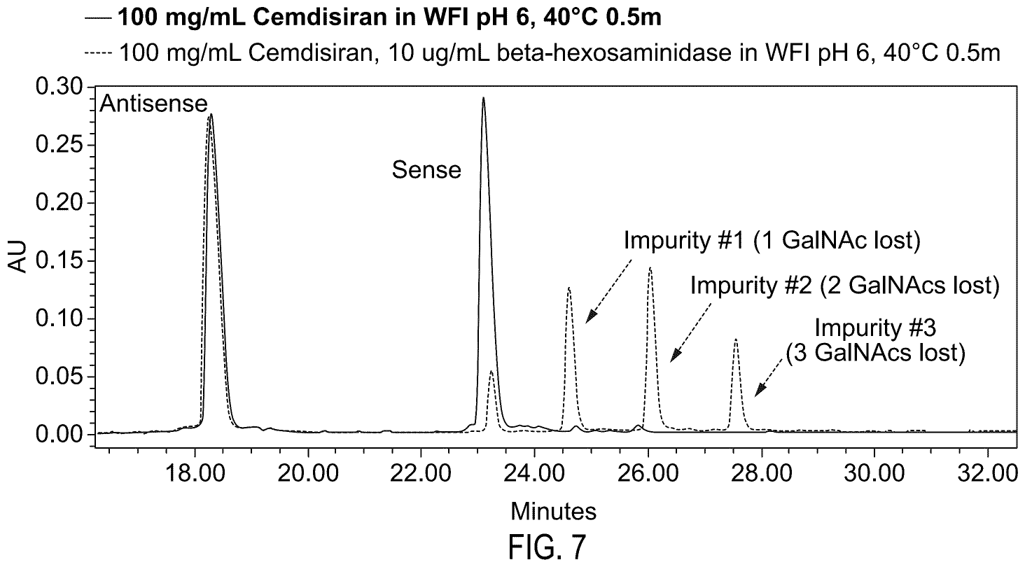

Experiment 2. A beta-hexosaminidase spiking study at accelerated conditions confirmed that the enzyme was responsible for Cemdisiran degradation in the liquid co-formulations. Cemdisiran formulations (100 mg/mL) (i.e., Cemdisiran reconstituted with WFI) with and without 10 μg/mL beta-hexosaminidase were manufactured. Glass vials were filled with formulation and stored at 40° C./75% RH. FIG. 7 shows the chromatograms from dIPRP analysis after 0.5 month at 40° C./75% RH of both formulations. A much smaller sense peak and 3 large impurities peaks were present (impurity #1, #2, and #3) in the stability sample containing beta-hexosaminidase. Impurity #1 and #2 were the same as demonstrated in FIG. 4: the loss of 1-2 GalNAc and an assumption is that impurity #3 is the loss of the third and final GalNAc.

### Reducing Cemdisiran Degradation

Experiment 1 (pH Study). Another stability study was initiated (similar to the set-up described for the above study of Cemdisiran+Pozelimab) with two co-formulations, both containing 50 mg/mL Cemdisiran and 100 mg/mL Pozelimab, both manufactured from the same source material (e.g., Pozelimab). However, an additional 20 mM of histidine was added to one co-formulation so that the final pH would be higher, pH 6.6. Excipients and final concentrations in the co-formulations studied in this Experiment are shown below:
50 mg/mL Cemdisiran, 100 mg/mL Pozelimab, 75 mM arginine HCl, 15 mM histidine, pH 5.9, 1.5% sucrose, 0.1125% PS80
50 mg/mL Cemdisiran, 100 mg/mL Pozelimab, 75 mM arginine HCl, 35 mM histidine, pH 6.6, 1.5% sucrose, 0.1125% PS80

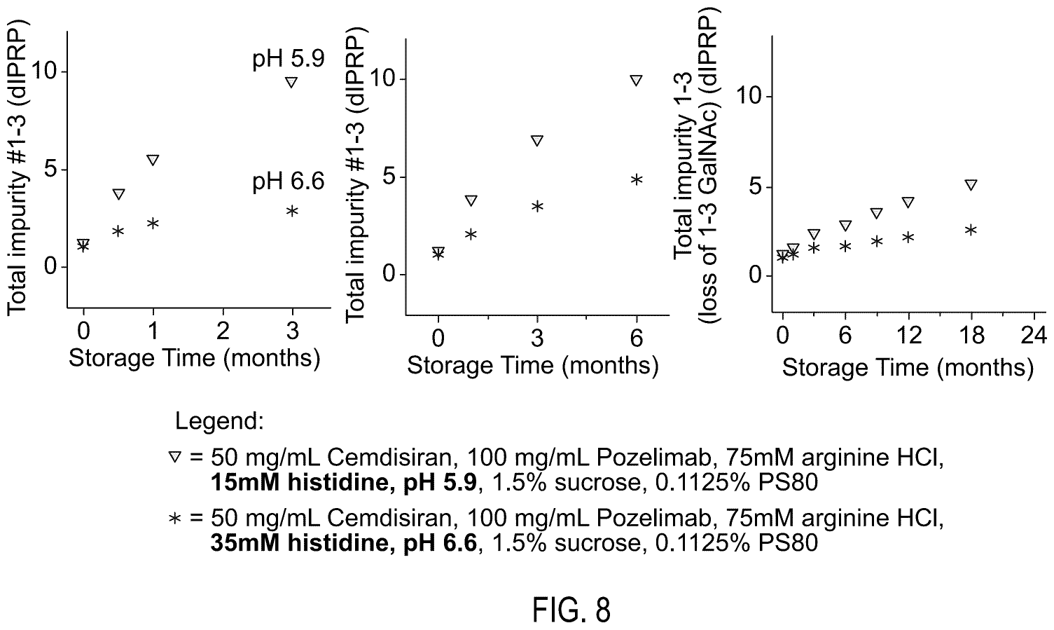

FIG. 8 showed the difference in % total impurities 1-3 (i.e., loss of 1-3 GalNAc) via dIPRP after storage at all three temperatures between the two co-formulations. Data shows that there was a significant decrease in Cemdisiran degradation, indicating that beta-hexosaminidase activity was pH dependent. At higher pH, beta-hexosaminidase activity was reduced.

Experiment 2. Another research stability study was initiated (similar to Cemdisiran+Pozelimab) with the following co-formulations:
50 mg/mL Cemdisiran, 100 mg/mL Pozelimab, 75 mM Arg HCl, 15 mM His, 1.5% sucrose, 0.1125% PS80, pH 6.0 with 5% GlcNAc;
50 mg/mL Cemdisiran, 100 mg/mL Pozelimab, 75 mM Arg HCl, 15 mM His, 1.5% sucrose, 0.1125% PS80, pH 6.0 with 5% GalNAc;
100 mg/mL Cemdisiran, 100 mg/mL Pozelimab, 50 mM Arg HCl, 10 mM His, 1.0% sucrose, 0.075% PS80, pH 6.0 with 5% GlcNAc;
   and
100 mg/mL Cemdisiran, 100 mg/mL Pozelimab, 50 mM Arg HCl, 10 mM His, 1.0% sucrose, 0.075% PS80, pH 6.0 with 5% GalNAc.

All co-formulations with GalNAc or GlcNAc showed no appreciable change in % total impurities 1-3 (i.e., loss of 1-3 GalNAcs from Cemdisiran) even after 6 months at 25° C. or 3 months at 40° C. See Tables 2-8-2-11.

TABLE 2-8
Stability of 50 mg/mL Cemdisiran & 100 mg/mL Pozelimab with GlcNAc Drug
Product After 25° C./60% RH Storage and 40° C./75% RH Storage (lot 3)

| | | 50 mg/mL Cemdisiran, 100 mg/mL Pozelimab, 15 mM | | | | | | |
|---|---|---|---|---|---|---|---|---|
| | | histidine, 75 mM arginine hydrochloride, 0.113% (w/v) | | | | | | |
| | | polysorbate 80, 1.5% (w/v) sucrose, pH 6.0 + 5% GlcNAc | | | | | | |
| | | Length of Storage (months) | | | | | | |
| Formulation | | | 25° C./60% RH | | | 40° C./75% RH | | |
| Assay | | t = 0 | 1 | 3 | 6 | 0.5 | 1 | 3 |
| Color and appearance^{a} | | Pass | Pass | Pass | Pass | Pass | Pass | Pass |
| pH | | 5.9 | 5.9 | 6.0 | 5.9 | 6.0 | 5.9 | 5.9 |
| Particulate | ≥10 μm | 8 | NR | NR | 62 | NR | NR | 31 |
| Analysis by | ≥25 μm | | | | | | | |
| MFI^{b} (#/mL) | | 0 | NR | NR | 8 | NR | NR | 0 |
| Cemdisiran Purity (%) by | | 90.9 | 90.1 | 90.9 | 90.4 | 89.9 | 89.7 | 89.5 |
| dIPRP | | | | | | | | |
| Total Impurities 1, 2, and | | 1.1 | 1.1 | 1.3 | 1.2 | 1.2 | 1.2 | 1.7 |
| 3 (%) by dIPRP^{c} | | | | | | | | |
| Total Pozelima^{b} | | 100 | 99 | 98 | 98 | 102 | 98 | 93 |
| Recovered (%) | | | | | | | | |
| Pozelimab | HMW | 1.0 | 1.2 | 1.4 | 1.6 | 1.8 | 2.6 | 6.9 |
| Purity by SE- | Main | 98.5 | 98.4 | 97.9 | 97.5 | 97.7 | 96.8 | 91.6 |
| | peak | | | | | | | |
| FLR-UPLC (%) | LMW | 0.5 | 0.4 | 0.7 | 0.9 | 0.5 | 0.6 | 1.4 |
| Pozelimab | Region 1 | 31.7 | 33.1 | 35.6 | 33.8 | 37.3 | 46.7 | 67.7 |
| Charge Variant | Region 2 | 57.5 | 53.7 | 48.5 | 51.0 | 48.7 | 37.1 | 19.1 |
| Analysis by | Region 3 | 10.8 | 13.2 | 15.8 | 15.1 | 14.0 | 16.0 | 13.2 |
| iCIEF^{d} (%) | | | | | | | | |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak. dIPRP, denaturing ion pair reverse phase; FLR, fluorescence; GalNAc, N-acetylgalactosamine; GlcNAc, N-acetylglucosamine; HMW, high molecular weight; iCIEF, imaging capillary isoelectric focusing; LMW, low molecular weight; MFI, Micro-Flow Imaging; ndIPRP, nondenaturing ion pair reverse phase; NR, not required; SE, size exclusion; UPLC, ultra-performance liquid chromatography

TABLE 2-9
Stability of 50 mg/mL Cemdisiran & 100 mg/mL Pozelimab with GalNAc Drug
Product After 25° C./60% RH Storage and 40° C./75% RH Storage (lot 3)

| | | Formulation | | | | | | |
|---|---|---|---|---|---|---|---|---|
| | | 50 mg/mL Cemdisiran, 100 mg/mL Pozelimab, 15 mM histidine, 75 mM arginine | | | | | | |
| | | hydrochloride, 0.113% (w/v) polysorbate 80, 1.5% (w/v) sucrose, pH | | | | | | |
| | | 6.0 + 5% GalNAc | | | | | | |
| | | Length of Storage (months) | | | | | | |
| | | | 25° C./60% RH | | | 40° C./75% RH | | |
| Assay | | t = 0 | 1 | 3 | 6 | 0.5 | 1 | 3 |
| Color and appearance^{a} | | Pass | Pass | Pass | Pass | Pass | Pass | Pass |
| pH | | 5.9 | 5.9 | 5.9 | 5.9 | 6.0 | 5.9 | 5.9 |
| Particulate Analysis by | ≥10 μm | 100 | 102 | 98 | 99 | 102 | 102 | 99 |
| MFI^{b} (#/mL) | ≥25 μm | 12 | NR | NR | 31 | NR | NR | 16 |
| Cemdisiran Purity (%) by dIPRP | | 90.8 | 90.2 | 90.8 | 90.3 | 89.5 | 89.6 | 89.1 |
| Total Impurities 1, 2, and 3 (%) | | 1.1 | 1.1 | 1.2 | 1.0 | 1.1 | 1.1 | 1.3 |
| by dIPRP^{c} | | | | | | | | |
| Pozelimab Purity by | HMW | 1.0 | 1.1 | 1.4 | 1.7 | 1.9 | 2.8 | 8.1 |
| SE-FLR-UPLC (%) | Main peak | 98.6 | 98.4 | 97.9 | 97.4 | 97.5 | 96.5 | 90.4 |
| | LMW | 0.4 | 0.4 | 0.7 | 0.9 | 0.6 | 0.6 | 1.5 |
| Pozelimab Charge Variant | Region 1 | 31.5 | 32.3 | 37.2 | 36.2 | 38.4 | 47.7 | 72.1 |
| Analysis by iCIEF^{d} (%) | Region 2 | 57.6 | 53.6 | 47.8 | 47.6 | 46.5 | 36.3 | 16.7 |
| | Region 3 | 11.0 | 14.1 | 15.0 | 16.2 | 14.2 | 16.0 | 11.2 |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak.
dIPRP, denaturing ion pair reverse phase;
FLR, fluorescence;
GalNAc, N-acetylgalactosamine;
GlcNAc, N-acetylglucosamine;
HMW, high molecular weight;
iCIEF, imaging capillary isoelectric focusing;
LMW, low molecular weight;
MFI, Micro-Flow Imaging;
ndIPRP, nondenaturing ion pair reverse phase;
NR, not required;
SE, size exclusion;
UPLC, ultra-performance liquid chromatography

TABLE 2-10
Stability of 100 mg/mL Cemdisiran & 100 mg/mL Pozelimab with GlcNAc Drug
Product After 25° C./60% RH Storage and 40° C./75% RH Storage (lot 3)

| | | Formulation | | | | | | |
|---|---|---|---|---|---|---|---|---|
| | | 100 mg/mL Cemdisiran, 100 mg/mL Pozelimab, 10 mM histidine, 50 mM arginine | | | | | | |
| | | hydrochloride, 0.075% (w/v) polysorbate 80, 1.0% (w/v) sucrose, pH | | | | | | |
| | | 6.0 + 5% GlcNAc (months) | | | | | | |
| | | Length of Storage (months) | | | | | | |
| | | | 25° C./60% RH | | | 40° C./75% RH | | |
| Assay | | t = 0 | 1 | 3 | 6 | 0.5 | 1 | 3 |
| Color and appearance^{a} | | Pass | Pass | Pass | Pass | Pass | Pass | Pass |
| pH | | 5.9 | 5.9 | 6.0 | 5.9 | 6.0 | 5.9 | 5.9 |
| Particulate Analysis by | ≥10 μm | 0 | NR | NR | 73 | NR | NR | 46 |
| MFI^{b} (#/mL) | ≥25 μm | 0 | NR | NR | 54 | NR | NR | 8 |
| Cemdisiran Purity (%) by dIPRP | | 90.5 | 89.9 | 90.8 | 90.4 | 90.1 | 89.6 | 89.9 |
| Total Impurities 1, 2, and 3 (%) | | 1.1 | 1.1 | 1.3 | 1.2 | 1.1 | 1.2 | 1.6 |
| by dIPRP^{c} | | | | | | | | |
| Pozelimab Purity by | HMW | 1.1 | 1.5 | 1.9 | 2.2 | 2.4 | 3.5 | 8.6 |
| SE-FLR-UPLC (%) | Main peak | 98.4 | 97.9 | 97.2 | 96.9 | 96.8 | 95.7 | 90.1 |
| | LMW | 0.5 | 0.6 | 0.9 | 0.9 | 0.7 | 0.8 | 1.3 |
| Pozelimab Charge Variant | Region 1 | 31.0 | 32.3 | 36.9 | 38.5 | 35.4 | 48.1 | 70.9 |
| Analysis by iCIEF^{d} (%) | Region 2 | 58.3 | 54.1 | 49.1 | 48.0 | 51.2 | 37.7 | 18.7 |
| | Region 3 | 10.7 | 13.6 | 14.1 | 13.5 | 13.4 | 14.2 | 10.5 |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak.
dIPRP, denaturing ion pair reverse phase;
FLR, fluorescence;
GalNAc, N-acetylgalactosamine;
GlcNAc, N-acetylglucosamine;
HMW, high molecular weight;
iCIEF, imaging capillary isoelectric focusing;
LMW, low molecular weight;
MFI, Micro-Flow Imaging;
ndIPRP, nondenaturing ion pair reverse phase;
NR, not required;
SE, size exclusion;
UPLC, ultra-performance liquid chromatography

TABLE 2-11
Stability of 100 mg/mL Cemdisiran & 100 mg/mL Pozelimab with GalNAc Drug Product
After 25° C./60% RH Storage, 40° C./75% RH Storage (lot 3)

| | | Formulation | | | | | | |
|---|---|---|---|---|---|---|---|---|
| | | 100 mg/mL Cemdisiran, 100 mg/mL Pozelimab, 10 mM histidine, 50 mM arginine | | | | | | |
| | | hydrochloride, 0.075% (w/v) polysorbate 80, 1.0% (w/v) sucrose, pH | | | | | | |
| | | 6.0 + 5% GalNAc | | | | | | |
| | | Length of Storage (months) | | | | | | |
| | | | 25° C./60% RH | | | 40° C./75% RH | | |
| Assay | | t = 0 | 1 | 3 | 6 | 0.5 | 1 | 3 |
| Color and appearance^{a} | | Pass | Pass | Pass | Pass | Pass | Pass | Pass |
| pH | | 5.9 | 5.9 | 6.0 | 5.9 | 6.0 | 5.9 | 5.9 |
| Particulate Analysis by | ≥10 μm | 31 | NR | NR | 8 | NR | NR | NA^{e} |
| MFI^{b} (#/mL) | ≥25 μm | 4 | NR | NR | 0 | NR | NR | NA^{e} |
| Cemdisiran Purity (%) by dIPRP | | 91.1 | 90.0 | 91.0 | 90.7 | 90.0 | 89.7 | 89.9 |
| Total Impurities 1, 2, and 3 (%) | | 1.0 | 1.1 | 1.2 | 1.0 | 1.1 | 1.1 | 1.3 |
| by dIPRP^{c} | | | | | | | | |
| Pozelimab Purity by | HMW | 1.1 | 1.5 | 1.9 | 2.3 | 2.5 | 3.7 | 9.7 |
| SE-FLR-UPLC (%) | Main peak | 98.5 | 97.9 | 97.3 | 96.8 | 96.7 | 95.5 | 88.9 |
| | LMW | 0.5 | 0.6 | 0.9 | 0.9 | 0.8 | 0.8 | 1.4 |
| Pozelimab Charge Variant | Region 1 | 30.2 | 32.5 | 37.3 | 37.3 | 34.5 | 48.7 | 72.4 |
| Analysis by iCIEF^{d} (%) | Region 2 | 59.0 | 53.8 | 49.3 | 49.2 | 52.4 | 37.1 | 18.4 |
| | Region 3 | 10.8 | 13.7 | 13.3 | 13.5 | 13.1 | 14.3 | 9.2 |

^{a}Sample passes color and visual appearance assessment if it is clear to slightly opalescent, essentially free from visible particulates, and colorless to yellow.
^{b}Silicone oil droplets and bubbles removed by removing particles with aspect ratio ≤0.85.
^{c}Impurities 1, 2, and 3 result from loss of 1, 2, and 3 N-acetylgalactosamines (GalNAcs).
^{d}Region 1 corresponds to acidic species that elute before the main peak, Region 2 corresponds to the main peak, and Region 3 corresponds to basic species that elute after the main peak.
^{e}Sample result unavailable to due instrument issue
dIPRP, denaturing ion pair reverse phase;
FLR, fluorescence;
GalNAc, N-acetylgalactosamine;
GlcNAc, N-acetylglucosamine;
HMW, high molecular weight;
iCIEF, imaging capillary isoelectric focusing;
LMW, low molecular weight;
MFI, Micro-Flow Imaging;
ndIPRP, nondenaturing ion pair reverse phase;
NR, not required;
SE, size exclusion;
UPLC, ultra-performance liquid chromatography

Concentration of beta-hex in Pozelimab formulated drug substance (FDS) directly impacts Cemdisiran degradation rates in liquid co-formulations

Pozelimab with the low and high measured concentrations of beta-hex (lot 3 and lot 4, respectively) was used to manufacturing additional two co-formulations and placed in storage for a study of stability.

The two co-formulations were:
100 mg/mL Cemdisiran, 100 mg/mL Pozelimab, 50 mM Arg HCl, 10 mM His, 1.0% sucrose, 0.075% PS80, pH 6.0; and
50 mg/mL Cemdisiran, 100 mg/mL Pozelimab, 75 mM Arg HCl, 15 mM His, 1.5% sucrose, 0.1125% PS80, pH 6.0

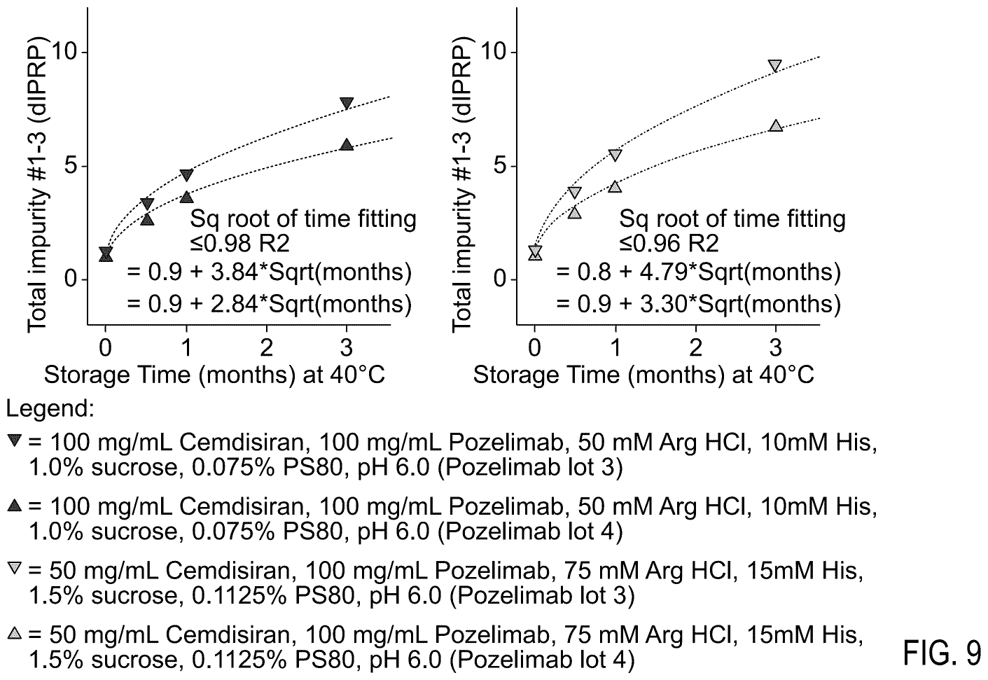

FIG. 9 shows that the co-formulations manufactured from Pozelimab containing the high level of beta-hexosaminidase exhibited a faster rate of Cemdisiran degradation than that of the low level of beta-hexosaminidase (i.e., higher rate of % total GalNac impurities via dIPRP). Sq root over time was found to have the best fitting for the data and equations.

Data also showed that the rate of Cemdisiran degradation was proportional to the beta-hexosaminidase content. Co-formulations manufactured with Pozelimab lot 3, (containing 30% higher beta-hex compared to lot 4), resulted in a 30-35% faster rate of Cemdisiran degradation for both co-formulations, compared to the co-formulations manufactured with Pozelimab lot 4.

### Example 3: Formulation Characterization and PS80 (Polysorbate 80) Ranging Studies Performed to Identity Co-Formulations

To optimize pH and the concentrations of the selected excipients for Cemdisiran & Pozelimab co-formulations, a Design of Experiment (DOE) study was developed to characterize and explore the excipient design space (see Table 3-1). A 23 co-formulation study was designed that considered all main effects, as well as the identified secondary interactions and quadratics, as defined by a risk assessment. The design was created, evaluated, and confirmed using statistical JMP software to be sufficiently powered and able to cover >95% design space with good estimation capability (i.e., prediction variance <1.0). Each co-formulation also contained 0.15% PS80.

TABLE 3-1
Formulation Parameters Used in the Cemdisiran &
Pozelimab Co-Formulation Characterization Study

| Factor | Low Level | High Level |
|---|---|---|
| pH | 5.5 | 7.0 |
| Sucrose Concentration (% w/v) | 0.8 | 3.6 |
| L-Arginine Hydrochloride Concentration (mM) | 40 | 140 |
| Pozelimab Concentration (mg/mL) | 90 | 110 |
| Cemdisiran Concentration (mg/mL) | 20 | 100 |
| Histidine Concentration (mM) | 35 | 45 |

An outline of the long-term, accelerated, and stress stability conditions, and freeze-thaw conditions is presented in Table 3-2.

TABLE 3-2
Formulation Characterization Stability
Studies for Cemdisiran + Pozelimab

| Long-Term | | Container/ |
|---|---|---|
| Stability | | Closure |
| Storage Temperature | Length of Storage (months) | glass vial |
| 5° C. | 0, 4, 6, 8, 12 | with elastomeric |
| | and 24 months | stopper |
| Accelerated and Stress Stability | | |
| Incubation Temperature | Length of Incubation | |
| 25° C./60% RH | 0, 2, and 3 months | |
| 40° C./75% RH | 0, 0.5, and 1 month | |

Excipients, excipient concentrations and pH were chosen based on the following desirability criteria:
minimized rate of HMW formation at 2-8° C.;
minimized rate of formation of acidic charge variants at 2-8° C.;
tonicity acceptable for SC administration; and
minimal Cemdisiran degradation at 2-8° C.

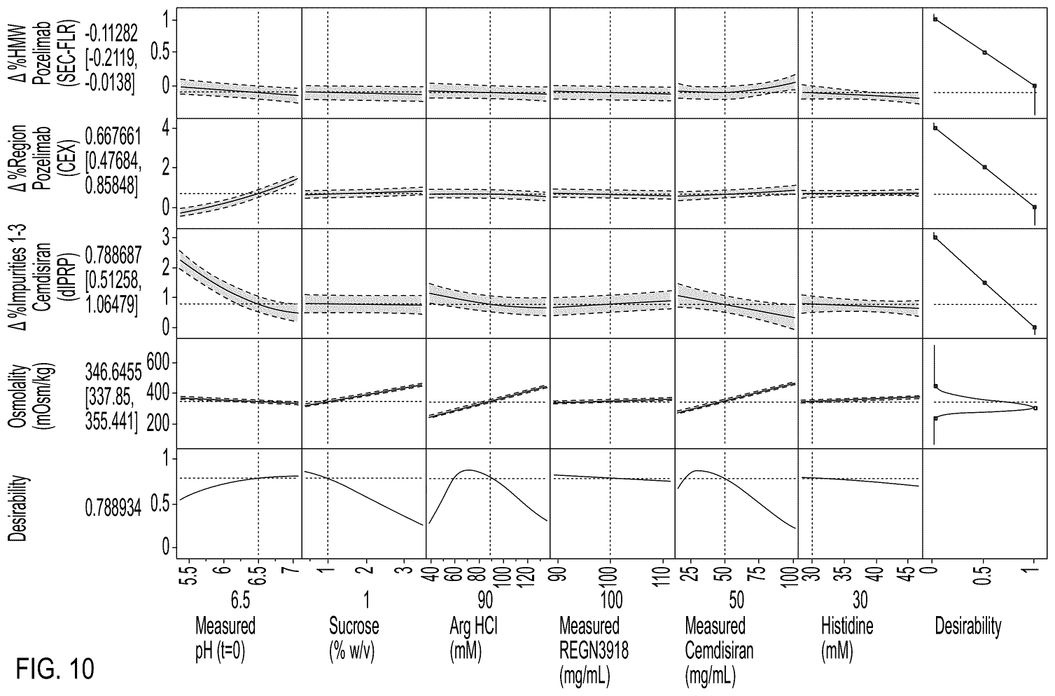

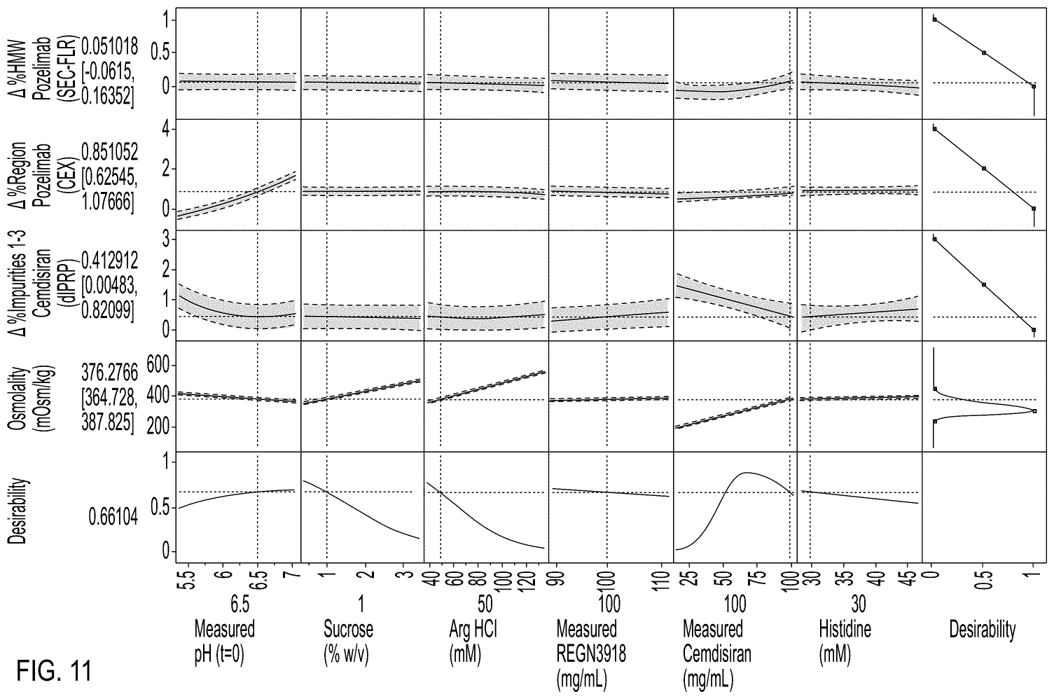

Excipients, excipient concentrations and pH displayed of co-formulations evaluated in the DOE experiments are set forth in FIG. 10 and FIG. 11. FIG. 10 represents the formulation composition that is set to the 50:100 (Cemdisiran:Pozelimab) optimized co-formulation. FIG. 11 represents the formulation composition that is set to the 100:100 optimized co-formulation. A desirability of 0.66 or 0.78 is considered a high value based on model and desirability criteria.

A higher pH (relative to pH 6) resulted in significant reduction in Cemdisiran degradation at 2-8° C. At a higher pH, a higher rate of Pozelimab acidic charge formation was observed, but this effect was relatively small after 2-8° C. storage for 4 months.

For the 50:100 co-formulation, a higher arginine HCl concentration (e.g., 90 mM vs 50 mM) was observed to decreased Cemdisiran degradation and allowed for a near isotonic solution.

### PS80 Ranging Studies

Once sucrose concentration, arginine HCl concentration, histidine concentration, and pH was selected for each co-formulation, an agitation stability study was performed to evaluate the concentration of PS80. Surfactant is necessary for preventing protein instability at air-liquid interface. The following co-formulations were manufactured with 0.025, 0.050, 0.075, 0.1 or 0.2% (w/v) PS80:
100 mg/mL Cemdisiran, 100 mg/mL Pozelimab, 50 mM arginine HCl, 30 mM histidine, 1% sucrose, X % PS80, pH 6.5; and
50 mg/mL Cemdisiran, 100 mg/mL Pozelimab, 90 mM arginine HCl, 30 mM histidine, 1% sucrose, X % PS80, pH 6.5

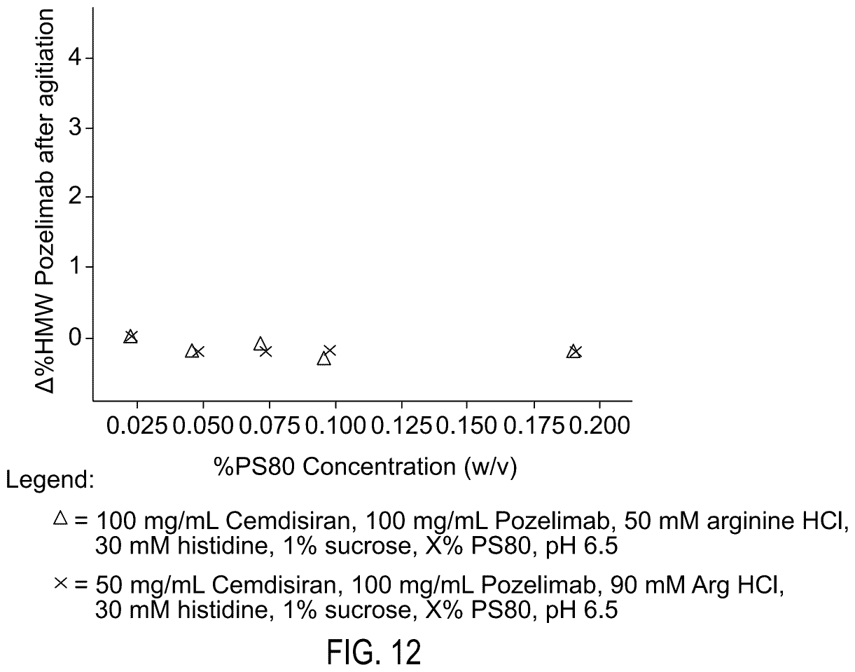

Glass vials were fill with co-formulation and placed on an orbital shaker in sideway orientation at 250 RPM for 48 hours. All co-formulations showed no change in any monitored quality attribute, including % HMW, as shown in FIG. 12.

### Stability of Optimized Co-Formulations

Research studies were initiated to assess the long-term, accelerated, and stress stability stress for the optimized co-formulations of Cemdisiran and Pozelimab in a single formulation. Glass syringes were filled with following co-formulation and placed in storage for a stability study:
100 mg/mL Cemdisiran, 100 mg/mL Pozelimab, 50 mM arginine HCl, 30 mM histidine, 1% sucrose, 0.075% PS80, pH 6.5; and
50 mg/mL Cemdisiran, 100 mg/mL Pozelimab, 90 mM arginine HCl, 30 mM histidine, 1% sucrose, 0.075% PS80, pH 6.5

Long-term, Accelerated, and Stress Stability Study. An outline of the long-term, accelerated, and stress stability conditions, and the agitation and freeze-thaw conditions is presented in Table 3-3.

TABLE 3-3
Research Stability Studies for Cemdisiran +
Pozelimab Optimized Assessment

| Long-Term | | Container/ |
|---|---|---|
| Stability | | Closure |
| Storage Temperature | Length of Storage (months) | glass syringes |
| 5° C. | 0, 1, 3, 6, 9, 12, | with elastomeric |
| | 18, 24, and 36 | stopper |
| Accelerated and Stress Stability | | |
| Incubation Temperature | Length of Incubation | |
| 25° C./60% RH | 0, 1, 2, 3, and 6 months | |
| 40° C./75% RH | 0, 0.25, 0.5, 1, | |
| | 2, and 3 months | |

No appreciable changes in the Pozelimab stability (e.g., formation of HMW and charge variants) or Cemdisiran stability (e.g., purity) after 6 months storage at 5° C. (see Table 3-4 and Table 3-5).

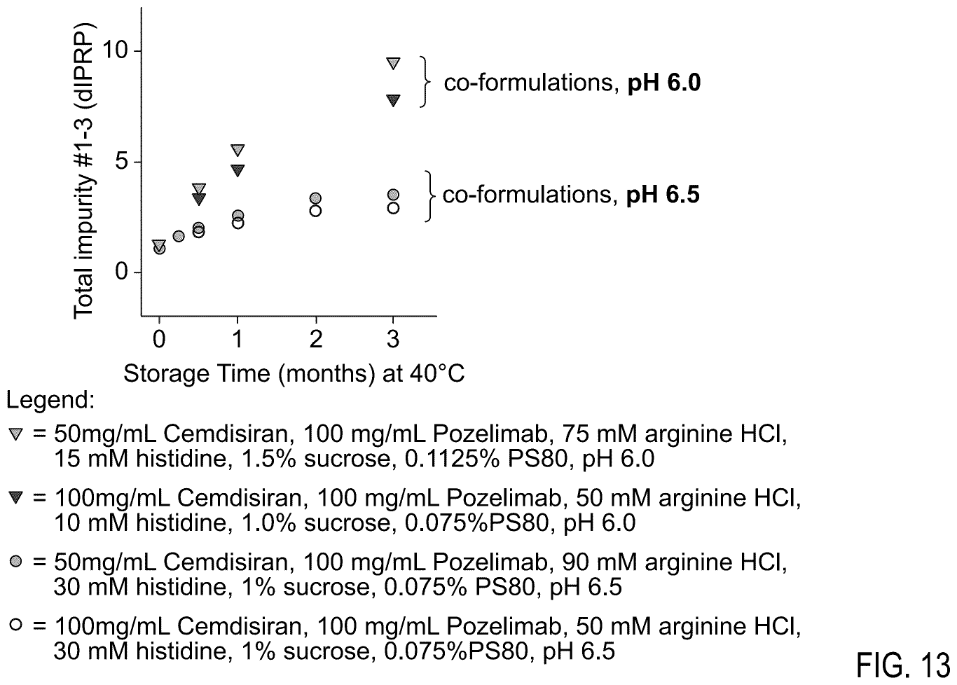

There was a decrease in Cemdisiran purity (by dIPRP) in both co-formulations at 4000/75% RH. However, the rate of Cemdisiran degradation was much lower in these co-formulations than in those of other studies (FIG. 13).

TABLE 3-4
Stability of 100 mg/mL Cemdisiran & 100 mg/mL Pozelimab Optimized Drug Product After 5° C. Storage

| | | DP Formulation | | | | | |
|---|---|---|---|---|---|---|---|
| | | 100 mg/mL Cemdisiran, 100 mg/mL Pozelimab, 30 mM histidine, 50 mM arginine | | | | | |
| | | hydrochloride, 0.075% (w/v) polysorbate 80, 1.0% (w/v) sucrose, pH 6.5 | | | | | |
| | | Pozelimab FDS Lot Number | | | | | |
| | | 3 | | | | | |
| | | Length of Storage (months) | | | | | |
| | | | 2-8° C. | | | | |
| Assay | | t = 0 | 1 | 3 | 6 | 9 | 12 |
| pH | | 6.6 | 6.5 | 6.6 | 6.6 | 6.6 | 6.6 |
| Particulate Analysis by | ≥10 μm | 81 | NR | NR | 89 | NR | 153 |
| MFI^{a} (#/container) | ≥25 μm | 49 | NR | NR | 0 | NR | 8 |
| Cemdisiran Purity (%) by dIPRP | | 90.1 | 90.7 | 90.7 | 90.9 | 90.1 | 89.6 |
| Total Impurities 1, 2, and 3 (%) | | 1.1 | 1.1 | 1.1 | 1.3 | 1.7 | 1.9 |
| by dIPRP^{b} | | | | | | | |
| Pozelimab Purity by | HMW | 1.1 | 1.1 | 1.2 | 1.4 | 1.4 | 1.6 |
| SE-FLR-UPLC (%) | Main peak | 98.1 | 98.1 | 98.0 | 97.8 | 97.8 | 97.7 |
| | LMW | 0.8 | 0.8 | 0.8 | 0.7 | 0.8 | 0.8 |
| Pozelimab Charge Variant | Region 1 | 33.5 | 34.1 | 30.5 | 31.4 | 31.1 | 30.1 |
| Analysis by iCIEF^{c} (%) | Region 2 | 57.4 | 56.6 | 59.8 | 59.3 | 59.2 | 59.6 |
| | Region 3 | 9.2 | 9.3 | 9.7 | 9.2 | 9.7 | 10.2 |

| | | | | | | |
|---|---|---|---|---|---|---|
| Pozelimab Relative Potency (%) | 100 | NR | NR | 134 | NR | 112 |

^{a}Silicone oil droplets and bubbles removed by removing particles with aspect ratio ≤0.85.
^{b}Impurities 1, 2, and 3 result from loss of 1, 2, and 3 N-acetylgalactosamines (GalNAcs).
^{c}Region 1 corresponds to acidic species that elute before the main peak, Region 2 corresponds to the main peak, and Region 3 corresponds to basic species that elute after the main peak.
dIPRP, denaturing ion pair reverse phase;
DP, drug product;
FDS, Formulated Drug Substance;
FLR, fluorescence;
HMW, high molecular weight;
iCIEF, imaging capillary isoelectric focusing;
LMW, low molecular weight;
Micro-Flow Imaging;
NR, not required;
SE, size exclusion;
UPLC, ultra-performance liquid chromatography

TABLE 3-5
Stability of 50 mg/mL Cemdisiran & 100 mg/mL Pozelimab Optimized Drug Product After 5° C. Storage

| | | DP Formulation | | | | | |
|---|---|---|---|---|---|---|---|
| | | 50 mg/mL Cemdisiran, 100 mg/mL Pozelimab, 30 mM histidine, 90 mM arginine | | | | | |
| | | hydrochloride, 0.075% (w/v) polysorbate 80, 1.0% (w/v) sucrose, pH 6.5 | | | | | |
| | | Pozelimab FDS Lot Number | | | | | |
| | | 3 | | | | | |
| | | Length of Storage (months) | | | | | |
| | | | 2-8° C. | | | | |
| Assay | | t = 0 | 1 | 3 | 6 | 9 | 12 |
| pH | | 6.6 | 6.5 | 6.5 | 6.5 | 6.5 | 6.5 |
| Particulate Analysis by | ≥10 μm | 169 | NR | NR | 9 | NR | 121 |
| MFI^{a} (#/container) | ≥25 μm | 33 | NR | NR | 0 | NR | 25 |
| Cemdisiran Purity (%) by dIPRP | | 90.5 | 90.4 | 90.4 | 90.6 | 89.7 | 88.9 |
| Total Impurities 1, 2, and 3 (%) | | 1.1 | 1.2 | 1.3 | 1.6 | 2.1 | 2.3 |
| by dIPRP^{b} | | | | | | | |
| Pozelimab Purity by | HMW | 0.9 | 1.0 | 1.0 | 1.1 | 1.1 | 1.2 |
| SE-FLR-UPLC (%) | Main peak | 98.4 | 98.2 | 98.3 | 98.2 | 98.1 | 98.2 |
| | LMW | 0.7 | 0.8 | 0.7 | 0.7 | 0.8 | 0.6 |
| Pozelimab Charge Variant | Region 1 | 33.5 | 33.8 | 32.1 | 31.9 | 22.9 | 24.4 |
| Analysis by iCIEF^{c} (%) | Region 2 | 56.2 | 56.8 | 58.7 | 58.8 | 65.9 | 65.2 |
| | Region 3 | 10.2 | 9.5 | 9.2 | 9.3 | 11.1 | 10.4 |

| | | | | | | |
|---|---|---|---|---|---|---|
| Pozelimab Relative Potency (%) | 83 | NR | NR | 96 | NR | 116 |

^{a}Silicone oil droplets and bubbles removed by removing particles with aspect ratio ≤0.85.
^{b}Impurities 1, 2, and 3 result from loss of 1, 2, and 3 N-acetylgalactosamines (GalNAcs).
^{c}Region 1 corresponds to acidic species that elute before the main peak, Region 2 corresponds to the main peak, and Region 3 corresponds to basic species that elute after the main peak.
dIPRP, denaturing ion pair reverse phase;
DP, drug product;
FDS, Formulated Drug Substance;
FLR, fluorescence;
HMW, high molecular weight;
iCIEF, imaging capillary isoelectric focusing;
LMW, low molecular weight;
Micro-Flow Imaging;
NR, not required;
SE, size exclusion;
UPLC, ultra-performance liquid chromatography

### Example 4: Clinical Trial Evaluating the Safety and Efficacy of Pozelimab and Cemdisiran Combination Therapy in Patients with Paroxysmal Nocturnal Hemoglobinuria (PNH) Who Switch from Pozelimab Monotherapy (R3918-PNH-2092)

PNH is an ultra-rare and life-threatening acquired genetic disease characterized by chronic intravascular hemolysis due to uncontrolled complement activation, and is associated with an increased risk of thrombosis. Patients with PNH often experience severe fatigue, which can negatively impact their physical functioning and health-related quality of life (QoL). Despite available treatments, patients may continue to experience episodes of breakthrough hemolysis due to insufficient complement inhibition. Cemdisiran and Pozelimab are therapeutic agents that act together to suppress terminal complement activity. Cemdisiran is an N-acetylgalactosamine-conjugated small interfering RNA (siRNA) that suppresses liver production of complement component C5, while Pozelimab is a fully human monoclonal antibody inhibitor of human C5. The combination of Pozelimab and Cemdisiran is being evaluated in an on-going phase 2, randomized, open-label, two-arm study (NCT04811716) that is designed to assess the safety and efficacy of Pozelimab and Cemdisiran combination therapy in PNH patients who have transitioned from Pozelimab monotherapy (30 mg/kg IV loading dose of Pozelimab, then 800 mg Pozelimab SC weekly) during an open-label extension trial (see NCT04162470). Safety and efficacy results are presented herein, along with patient-reported outcomes up to Week 16.

One objective of this study is to evaluate the safety and efficacy of two dosing regimens of Pozelimab and Cemdisiran combination therapy in patients with PNH who have transitioned from Pozelimab monotherapy. Another objective of this study is to report on patient-reported fatigue and impact on functioning and overall global health status (GHS)/QoL following treatment with Pozelimab and Cemdisiran combination therapy in patients with PNH who have transitioned from Pozelimab monotherapy.

TABLE 4-1
Baseline Characteristics

| | Arm 1 | | Arm 2 | |
|---|---|---|---|---|
| Characteristic | (n = 12) | | (n = 10) | |
| Age, years, mean (SD) | 53.2 | (16.4) | 43.9 | (17.2) |
| Sex, male, n (%) | 6 | (50.0) | 6 | (60.0) |
| Ethnicity, not Hispanic | 12 | (100.0) | 10 | (100.0) |
| or Latino, n (%) | | | | |
| Race, n (%) | | | | |
| White | 1 | (8.3) | 1 | (10.0) |
| Asian | 11 | (91.7) | 9 | (90.0) |
| PNH diagnosis age, | 45.0 | (32:50) | 29.5 | (24:57) |
| years, median (Q1:Q3) | | | | |
| Baseline LDH, U/L, mean (SD) | 237.9 | (41.1) | 237.2 | (49.1) |
| Baseline LDH, x ULN, mean (SD) | 0.8 | (0.2) | 0.8 | (0.2) |
| Hemoglobin, g/L, mean (SD) | 115.4 | (17.2) | 108.9 | (21.5)^{a} |
| eGFR, mL/min/1.73 m2, mean (SD) | 104.1 | (21.9) | 116.3 | (19.6) |
| Total bilirubin, μmol/L, mean (SD) | 22.8 | (12.3) | 33.0 | (16.8) |
| Haptoglobin, g/L, mean (SD) | 0.1 | (0.1) | 0.1 | (0.1) |
| Complement CH50, kIU/L, mean (SD) | 0.1 | (0.3) | 0.0 | (0.0) |

^{a}n = 9
Arm 1: Pozelimab 400 mg SC Q4W + Cemdisiran 200 mg SC Q4W. Arm 2: Pozelimab 400 mg SC Q2W + Cemdisiran 200 mg SC Q4W. CH50, total complement hemolysis assay; eGFR, estimated glomerular filtration rate; LDH, lactate dehydrogenase; PNH, paroxysmal nocturnal hemoglobinuria; Q, quartile; SC, subcutaneous; SD, standard deviation; ULN, upper limit of normal.

### Inclusion Criteria

A patient must meet the following criteria to be eligible for inclusion in the study:
1. Patients with PNH who are receiving treatment with Pozelimab monotherapy in the R3918-PNH-1868 study (NCT04162470; “An open-label extension study to evaluate the long-term safety, tolerability, and efficacy of REGN3918 in patients with paroxysmal nocturnal hemoglobinuria”) [Inclusion criterion for R3918-PNH-1868: Patients with PNH who have completed, without discontinuation, study treatment in one of the parent studies in which they participated (either R3918-PNH-1852 [NCT03946748; “Study to Evaluate the Efficacy and Safety of REGN3918 in Patients With Paroxysmal Nocturnal Hemoglobinuria (PNH)” including patients that are complement inhibitor-naïve or have not recently received complement inhibitor therapy] or R3918-PNH-1853 [“An Open-Label Study to Evaluate the Efficacy and Safety of REGN3918 in Patients with Paroxysmal Nocturnal Hemoglobinuria who Switch from Other Anti-C5 Therapies” including patients with PNH currently being treated with Eculizumab and who plan to switch to REGN3918]).
2. Provide informed consent signed by study patient
3. Willing and able to comply with clinic/remote visits and study-related procedures

### Exclusion Criteria

A patient who meets any of the criteria listed below will be excluded from the study. Patients may be re-screened up to 2 times after discussion between the investigator and the sponsor.
1. Documented*, positive polymerase chain reaction (PCR) or equivalent test based on regional recommendations for COVID-19 or suspected SARS-CoV-2 infection and:
   a. Have not recovered from COVID-19 (i.e., all COVID-19-related symptoms and major clinical findings which can potentially affect the safety of the patient have not been resolved), and
   b. Did not have 2 negative results from a nucleic acid amplification (PCR) test or equivalent test based on regional recommendations for COVID-19 to confirm that the patient is negative for SARS-CoV-2 or, if COVID-19 PCR (or equivalent) testing is not feasible, at least 3 months have transpired since the initial diagnosis
* Note: Screening for COVID-19 will not be performed as part of eligibility assessments for this study
2. Patients with documented history of liver cirrhosis or patients with liver disease with evidence of currently impaired liver function, or patients with ALT or AST (unrelated to PNH) >3×ULN at the screening visit (1 repeat lab is allowed during screening)
3. Significant protocol deviation(s) in the parent study based on the investigator's judgment and to the extent that these would (if continued) impact the study objectives and/or safety of the patient (for example, repetitive non-compliance with dosing by the patient)
4. Any new condition or worsening of an existing condition which, in the opinion of the investigator, would make the patient unsuitable for enrollment or would jeopardize the safety of the patient
5. Known hypersensitivity to Cemdisiran or any component of Cemdisiran formulation
6. Pregnant or breastfeeding women
7. Women of childbearing potential (WOCBP)* who are unwilling to practice highly effective contraception prior to the initial dose/start of the first treatment, during the study, and for at least 52 weeks after the last dose. Highly effective contraceptive measures include:
a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
b. intrauterine device (IUD); intrauterine hormone-releasing system (IUS);
c. bilateral tubal ligation;
d. vasectomized partner (provided that the male vasectomized partner is the sole sexual partner of the WOCBP study participant and that the vasectomized partner has obtained medical assessment of surgical success for the procedure); and/or
e. sexual abstinence†, ‡.
*WOCBP are defined as women who are fertile following menarche until becoming postmenopausal, unless permanently sterile. Permanent sterilization methods include hysterectomy, bilateral salpingectomy, and bilateral oophorectomy.

A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance (CTFG, 2020). Pregnancy testing and contraception are not required for women with documented hysterectomy or tubal ligation.

†Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient.
‡Periodic abstinence (calendar, symptothermal, post-ovulation methods), withdrawal (coitus interruptus), spermicides only, and lactational amenorrhea method (LAM) are not acceptable methods of contraception.
Note: The use of a female or male condom is not sufficient as a contraceptive measure but may be considered for the safety or prevention of sexually transmitted diseases. Female condom and male condom should not be used together

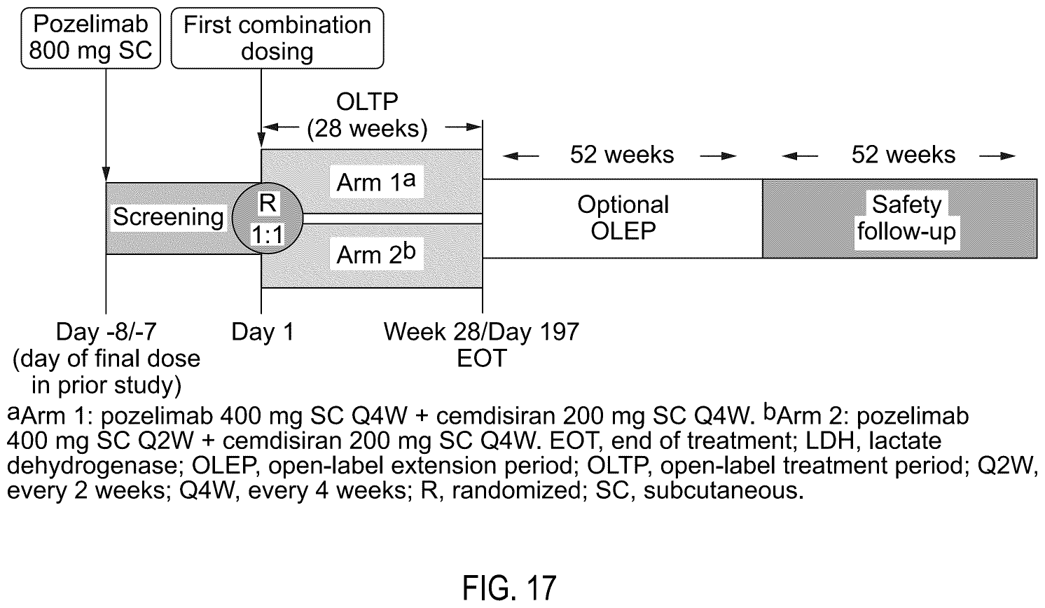

Methods: Patients (n=22) are randomized (1:1) into one of two treatment arms; both arms received subcutaneous (SC) Cemdisiran 200 mg every 4 weeks (Q4W) plus Pozelimab 400 mg SC at a frequency of either Q4W (arm 1) or every 2 weeks (Q2W; arm 2). The study consists of four periods:
a screening period (7-8 days),
an open-label treatment period (OLTP; 28 weeks),
an optional open-label extension period (OLEP; 52 weeks), and
a safety follow-up period (52 weeks).
   At any time during the study, patients meeting criteria for breakthrough hemolysis with sustained elevations of lactate dehydrogenase (LDH; >1.5× upper limit of normal [ULN] on two consecutive measurements spanning at least 2 weeks) are switched to an intensified Pozelimab treatment regimen. See FIG. 17.

The screening period is approximately 7 to 8 days. The duration of the OLTP (main study period) for a patient is a minimum of approximately 28 weeks. The duration will be longer for patients who require treatment intensification, which consists of a 28-week treatment regimen starting from the day of intensification. Patients who do not continue into the optional OLEP will be followed for an additional 52 weeks after the last dose of combination treatment. Patients opting to participate in the OLEP will continue open-label treatment for a 52-week period, to be followed by a 52-week safety follow-up period after the last dose of study treatment. Patients who complete the optional OLEP may be able to continue study treatment in a post-trial access program. Patients participating in the post-trial access program will therefore not be followed in the safety follow-up period.

Screening (7 to 8 days): The dose of Pozelimab is administered at the study site on the day of the screening visit in this study, which occurs on the day of a planned administration with Pozelimab monotherapy. Subsequently, the day 1 visit for the first combination treatment dosing occurs 7 to 8 days after the last dose of Pozelimab monotherapy.

As part of risk mitigation for this study, patients need to have documented vaccination against Neisseria meningitidis and receive updated meningococcal vaccination if needed. Daily oral antibiotic prophylaxis is recommended throughout the study. Patients are counseled regarding risk of infection with Neisseria gonorrhea, as applicable based on their risk level.

In addition to screening procedures to determine eligibility, patients are asked to complete a PNH Symptom-Specific Questionnaire daily for 7 consecutive days prior to the day 1 visit. Patients could choose to participate in the optional OLEP (open label extension period), optional future biomedical research, and/or optional pharmacogenomics component of the study by signing the respective optional informed consent forms.

Treatment Period (Day 1 to Week 28): Day 1 is scheduled 7 to 8 days after the last dose of Pozelimab monotherapy. On day 1, after confirming eligibility, patients are randomized in a 1:1 ratio to 1 of the 2 arms:
Arm 1: Pozelimab 400 mg SC every 4 weeks (Q4W) and Cemdisiran 200 mg SC Q4W;
Arm 2: Pozelimab 400 mg SC every 2 weeks (Q2W) and Cemdisiran 200 mg SC Q4W. The last doses of study treatment are administered at week 24 for arm 1 and week 26 for arm 2. Patients return for safety, efficacy, and other assessments at week 28. See Tables 4-2-4-5.

Optional Open-Label Extension Period (OLEP): All patients who complete the OLTP (including patients who receive intensified treatment) are offered the opportunity to continue in an optional 52-week OLEP, whereby the transition of the combination treatment from the OLTP to the OLEP is planned to be uninterrupted (i.e., day 1 visit of the OLE will correspond to the end of treatment (EOT) visit of the OLTP).

Patients whose treatment is not intensified during the main study period transition to the OLEP on a regimen of Pozelimab 400 mg SC Q4W and Cemdisiran 200 mg SC Q4W, regardless of their treatment assignment in the main treatment period. Patients whose treatment is intensified during the main study period transition to the OLEP and continue on the intensified treatment regimen of Pozelimab 400 mg SC Q2W and Cemdisiran 200 mg SC Q4W.

The OLEP ends 52 weeks after the first dose of study treatment in the OLEP, even if the patient requires intensified treatment during the OLEP. For patients who complete the optional OLEP, post-trial access to treatment may be available.

Combining pozelimab and Cemdisiran will rapidly and continuously suppress concentrations of C5 to pharmacologically inactive levels. A target-mediated drug disposition (TMDD) population PK model for Pozelimab and a population PK/PD model for Cemdisiran were developed based on respective data on healthy subjects. The models for Pozelimab and Cemdisiran were combined by introducing 05 production suppression effect of Cemdisiran to the synthesis rate of 05 in the TMDD model for Pozelimab. The unified model was used to perform simulations to inform dose selection of Pozelimab in combination with Cemdisiran. Based on simulations, Pozelimab 400 mg SC Q4W and Cemdisiran 200 mg SC Q4W will be sufficient to maintain the suppression of 05 to biologically inactive levels. In addition, at steady-state, the simulated total and free pozelimab concentration-time profiles are superimposable. This is consistent with the extremely low concentration of free 05 predicted by the unified population PK/PD model.

See the schedule of the various doses administered in Table 4-2 to 4-5 below.

TABLE 4-2
Schedule of Events (Open-Label Treatment Period)

| Screening | | | | | | | | | | | | |
|---|---|---|---|---|---|---|---|---|---|---|---|---|
| Period | Open-Label Treatment Period³ | | | | | | | | | | | |
| Study Procedure (Visit)^{1, 2} | | | | | | | | | | | | |
| Screening | | | | | | | | | | | | EOT⁵ |
| V1 | V2⁴ | V3 | V4 | V5 | V6 | V7 | V8 | V9 | V10 | V11 | V12 | V13 |
| Week | | | | | | | | | | | | |
| Up to −1 | 0 | 1 | 2 | 4 | 6 | 8 | 10 | 12 | 16 | 20 | 24 | 28 |
| Day | | | | | | | | | | | | |
| Up to −8 | 1 | 8 | 15 | 29 | 43 | 57 | 71 | 85 | 113 | 141 | 169 | 197 |
| Window (day) | | | | | | | | | | | | |
| — | — | ±3 | ±3 | ±3 | ±3 | ±3 | ±3 | ±3 | −7/+3 | −7/+3 | −7/+3 | −7/+3 |

Screening/Baseline:

| | | | | | | | | | | | | | |
|---|---|---|---|---|---|---|---|---|---|---|---|---|---|
| Inclusion/Exclusion | x | x | | | | | | | | | | | |
| Informed consent | x | | | | | | | | | | | | |
| Informed consents for OLEP, | x | | | | | | | | | | | | |
| FBR, and genomics research | | | | | | | | | | | | | |
| (optional) | | | | | | | | | | | | | |
| Medical history ⁶ | x | | | | | | | | | | | | |
| Prior medications ⁷ | x | | | | | | | | | | | | |
| Demographics | x | | | | | | | | | | | | |
| Height | x | | | | | | | | | | | | |
| Documentation of vaccination | x | | | | | | | | | | | | |
| for Neisseria meningitidis (or | | | | | | | | | | | | | |
| revaccination) ⁸ | | | | | | | | | | | | | |
| Risk assessment for | x | | | | | | | | | | | | |
| Neisseria gonorrhea ⁹ | | | | | | | | | | | | | |
| Patient safety card for | x | x | x | x | x | x | x | x | x | x | x | x | x |
| Neisseria meningitidis ¹⁰ | | | | | | | | | | | | | |
| Randomization | | x | | | | | | | | | | | |

Study Treatment:

| Arm 1 only: Pozelimab 400 mg | | x¹² | | | x | | x | | x | x | x | x | |
|---|---|---|---|---|---|---|---|---|---|---|---|---|---|
| SC Q4W ¹¹ | | | | | | | | | | | | | |
| Arm 2 only: Pozelimab 400 mg | | x¹² | | x | x | x | x | x | x | x | x | x¹¹ | |
| SC Q2W ¹¹ | | | | | | | | | | | | | |
| Cemdisiran 200 mg SC Q4W ¹¹ | | x¹² | | | x | | x | | x | x | x | x | |
| Injection training/patient | | <---------------------------------------------x---------------------------------------> | | | | | | | | | | | |
| instructions (as needed) ¹³ | | | | | | | | | | | | | |
| Patient diary ¹⁴ | x ¹⁵ | x | x | x | x | x | x | x | x | x | x | x | x |

| | |
|---|---|
| Antibiotics prophylaxis | <---------------------------------------------x---------------------------------------> |
| (recommended) ¹⁶ | |
| Revaccination against | <---------------------------------------------x---------------------------------------> |
| meningococcal infection (if needed) | |

Clinical Outcome Assessments:

| FACIT-Fatigue | | x | x | x | x | x | x | x | x | x |
|---|---|---|---|---|---|---|---|---|---|---|
| EORTC-QLQ-C30 | | x | x | x | x | x | x | x | x | x |
| TSQM | | x | x | x | x | x | x | x | x | x |
| PNH symptom-specific | <---------------------------------------------x---------------------------------------> | | | | | | | | | |
| questionnaire (daily) ¹⁷ | | | | | | | | | | |
| PGIS | | x | | x | | x | | x | | x |
| PGIC | | | | x | | x | | x | | x |

Safety and Anthropometric:

| | | | | | | | | | | | | | |
|---|---|---|---|---|---|---|---|---|---|---|---|---|---|
| Body weight | x | | | | x | | x | | x | x | x | x | x |
| Vital signs | x | | | x | x | | x | | x | x | x | x | x |
| Physical examination | x | | | | x | | | | x | | | | x |
| Electrocardiogram | x | | | | | | | | | | | | x |
| Adverse events | x | x | x | x | x | x | x | x | x | x | x | x | x |
| Breakthrough hemolysis | x | x | x | x | x | x | x | x | x | x | x | x | x |
| assessment ¹⁸ | | | | | | | | | | | | | |
| Concomitant meds/treatments | x | x | x | x | x | x | x | x | x | x | x | x | x |
| Transfusion record update | x | x | x | x | x | x | x | x | x | x | x | x | x |

Laboratory Testing¹⁹:

| | | | | | | | | | | | | | |
|---|---|---|---|---|---|---|---|---|---|---|---|---|---|
| Titers to measure N. meningitidis | x | | | | | | | | | | | | |
| (only if required per parent study) | | | | | | | | | | | | | |
| Coagulation panel | x | x | x | x | x | x | x | x | x | x | x | x | x |
| Chemistry (long panel) | x | x | x | x | x | x | x | x | x | x | x | x | x |
| including LDH ²⁰ | | | | | | | | | | | | | |
| Hematology ²¹ | x | x | x | x | x | | x | | x | x | x | x | x |
| Immunoglobulin G | | x | | | x | | | | x | | | | |
| Pregnancy test (WOCBP only): | s | u | | | u | | u | | u | u | u | u | u |
| serum (S) or urine (U) | | | | | | | | | | | | | |
| Urinalysis | x | x | x | x | x | | x | | x | x | x | x | x |

Pharmacokinetics and Immunogenicity Sampling:

| | | | | | | | | | | | |
|---|---|---|---|---|---|---|---|---|---|---|---|
| Pozelimab conc. sample ²² | x | x | x | x | x | x | x | x | x | x | x |
| Cemdisiran and its | | x | | | x | | x | | | x | |
| metabolite conc. sample ²³ | | | | | | | | | | | |
| Pozelimab immunogenicity | x | | | | | | x | | | | x |
| sample²⁴ | | | | | | | | | | | |
| Cemdisiran immunogenicity | | x | | | | | x | | | | x |
| sample²⁴ | | | | | | | | | | | |
| Total C5 (plasma) ²² | x | x | x | x | x | x | x | x | x | x | x |

Biomarkers:

| | | | | | | | | | | | |
|---|---|---|---|---|---|---|---|---|---|---|---|
| Free hemoglobin | x | x | | | x | x | x | x | x | x | x |
| Haptoglobin | x | x | | | x | | x | | | | x |
| Complement hemolytic assay | x | x | x | x | x | x | x | x | x | x | x |
| (serum CH50) ²⁵ | | | | | | | | | | | |
| Complement hemolytic assay | x | x | x | x | x | x | x | x | x | x | x |
| (serum AH50) ²⁵ | | | | | | | | | | | |
| sC5b-9 (plasma) | x | x | x | x | x | x | x | x | x | x | x |
| PNH erythrocyte cells | x | | | | | x | | | x | | x |
| PNH granulocyte cells | x | | | | | x | | | x | | x |

Optional research:

| | | | | | | |
|---|---|---|---|---|---|---|
| Serum and plasma | x | | | x | x | x |
| for FBR (optional) | | | | | | |
| Whole blood sample for DNA | | x | | | | |
| isolation for genomics | | | | | | |
| research (optional) ²⁶ | | | | | | |
| Whole blood RNA sample for | | x | x | | | x |
| genomics research (optional) | | | | | | |

Footnotes
¹Visits between week 6 and up to week 24 may be at the clinical site, or another preferred location, such as the patient's home. The location will be dependent on availability of home healthcare visiting professional as well as the preferences of the investigator and patient. In the event of travel restrictions due to a global pandemic, alternative mechanisms such as but not limited to telemedicine visits may be implemented to maintain continuity of study conduct.
²When multiple procedures are performed on the same day, the sequence of procedures is as follows: Clinical outcome assessments (COAs) → Electrocardiogram (ECG) → vital signs, physical examination, safety monitoring, lab collection → study treatment administration → any pre-specified post-dose sample collection.
³Patients who are re-started on an intensified treatment will undergo an adjustment to their scheduled visits. Patients may require unscheduled visit(s) as needed and should be subsequently followed per the Schedule of Events for Patients on Intensified Treatment in the OLTP.
⁴The day 1 visit should take place 7 to 8 days after the screening visit. Patients may be rescreened if they cannot schedule the screening visit and the day 1 visit over a period of 7 to 8 days.
⁵If the patient agrees to continue into the optional OLEP, the End of Treatment (EOT) visit of the OLTP will correspond to the day 1e visit of the OLEP (see Schedule of Events (Optional Open-Label Extension Period)). Any common assessments will be performed once for both visits.
⁶ Transfusions, breakthrough hemolysis history, and laboratory parameters for measurement of hemolysis (such as LDH, bilirubin, haptoglobin, reticulocyte count, and hemoglobin) should be obtained for the past 52 weeks, if possible. Prior history of thrombosis and Neisseria infections will be collected. Ongoing PNH symptoms and signs will also be collected. Information collected from parent studies may be used whenever possible.
⁷ Including Pozelimab administration.
⁸ Patients will have had previous documented vaccination for meningococcus (serotypes A, C, Y, W and serotype B if available) in the parent study, R3918-PNH-1868 (“An open-label extension study to evaluate the long-term safety, tolerability, and efficacy of REGN3918 in patients with paroxysmal nocturnal hemoglobinuria”), but may be revaccinated if prior vaccination is more than 5 years from screening. Alternatively, patients may be re-vaccinated in accordance with current national vaccination guidelines for vaccination use with complement inhibitors or local practice. Patients who require revaccination may be rescreened.
⁹ A risk factor assessment for Neisseria gonorrhea infection is recommended, and counseling is advised for at-risk patients.
¹⁰ A patient safety card will be distributed to patients at screening and risk information will be reviewed. Replacement cards may be given to the patient as needed.
¹¹ During OLTP, the dose of Cemdisiran and Pozelimab SC should be given on the day of the corresponding study visit whenever possible. Study treatment administration should always be the last procedure after all blood sample collection and study assessments have been completed unless otherwise specified.
If Pozelimab or Cemdisiran cannot be administered on the day of the corresponding study visit, the combination may be administered up to 3 days before or up to 3 days after the planned dosing date for Q2W dosing, provided that the combination dosing takes place after the corresponding study visit has been completed. For patients receiving Pozelimab 400 mg SC Q2W + Cemdisiran 200 mg SC Q4W, the dosing window (±3 days) is the same or narrower than the visit window (±3 days before week 16 or −7/+3 days on and after week 16).
If Pozelimab or Cemdisiran cannot be administered on the day of the corresponding study visit, the combination may be administered up to 7 days before or 7 days after the planned dosing date for Q4W dosing, provided that the combination dosing takes place after the corresponding study visit has been completed. For patients receiving Pozelimab 400 mg SC Q4W + Cemdisiran 200 mg SC Q4W, the visit window (±3 days before week 16 or −7/+3 days starting from week 16) is narrower than the dosing window (±7 days). Care must be taken to coordinate dosing for visits where a post-dose sample is collected to measure the concentration of Cemdisiran and its metabolites. The final SC dosing of the combination (Pozelimab and Cemdisiran) during the OLTP is at week 24 for arm 1 and the final SC dosing of Cemdisiran is at week 24 with Pozelimab at week 26 for arm 2.
¹²Patients should be monitored for at least 30 minutes after completing the first Cemdisiran injection. A 30 minute monitoring period is not needed after the Pozelimab injection.
¹³ Injection training will be provided to patients who desire self-injection or injection by a designated person. Sites should observe patient self-injection or injection by a designated person and confirm adequacy. Patient instruction materials will be provided. SC injections may either be performed by the site personnel or another healthcare professional at the patient's home or preferred location, or be administered by the patient or a designated person who has successfully completed the injection training.
¹⁴ If study treatment is given by the patient or by a designated person, the patient will complete a diary for recording compliance with study treatment administration. If patient diary is provided to the patient, then it should be reviewed at each clinic visit and data collected into the case report forms (CRFs). On the final visit, the diary should be collected by the site.
¹⁵ At the screening visit, patient diary should be reviewed for the R3918-PNH-1868 (parent) study.
¹⁶ Daily oral antibiotic prophylaxis against Neisseria meningitidis is recommended starting on the first day of dosing with study treatment and continuing until 52 weeks after discontinuation of study treatment.
¹⁷ Patient will complete daily PNH Symptom-Specific Questionnaire for 7 consecutive days prior to day 1 visit. Patients should try to complete the PNH Symptom-Specific Questionnaire at the same time each day whenever possible.
¹⁸ Breakthrough hemolysis assessment: If a patient is suspected of having a breakthrough hemolysis event, then in addition to the required laboratory collection, additional samples will be collected unless already noted in the schedule of events for that visit. If the suspected event does not occur at a scheduled visit, then an unscheduled visit should occur with an evaluation of the patient and collection of samples for suspected breakthrough hemolysis assessment. Unscheduled blood collection for suspected breakthrough hemolysis events should include, at a minimum, CBC, reticulocyte count, chemistry, coagulation parameters, D-dimer, total C5, CH50, ADA (against Pozelimab), and drug concentrations of Pozelimab, as applicable.
¹⁹Clinical lab samples will be collected prior to any study drug administration (pre-dose) unless otherwise specified. The same methodology will be applied across study visits for lab sample collection, handling and processing, as best as possible, to preserve the quality of samples and minimize hemolysis. The coagulation blood sample (tube) must always be collected first, followed by the blood chemistry sample (tube).
²⁰ Serum LDH, C-reactive protein (CRP), and bilirubin (total and direct) will be assessed as part of the blood chemistry analysis. Blood chemistry sample should be collected before study treatment administration (pre-dose). During lab collection, handling and processing, the same methodology will be applied across study visits, as best as possible, to preserve the quality of sample and avoid hemolysis during sample processing. If the investigator or sponsor suspects that the lab result is not an accurate reflection of the patient's condition, the lab sample should be repeated.
²¹ Hematology sample should be collected before study treatment administration (pre-dose).
²² Blood samples for Pozelimab concentration analysis and total C5 analysis will be obtained on the specified days prior to any study treatment administration (pre-dose).
²³ Blood samples for Cemdisiran concentration analysis and concentrations of its metabolites will be collected on the specified days prior to any study treatment administration (pre-dose) and 2 to 6 hours post Cemdisiran administration. The post-dose sample should be carefully coordinated with the dosing of Cemdisiran and may be collected at the clinic or by a visiting health care professional.
²⁴Blood samples for immunogenicity will be collected on the specified days prior to any study treatment administration (pre-dose). At the visits where immunogenicity samples are to be taken, the sample should be collected with the sample for drug concentration. In the event of suspected treatment-related SAEs, such as anaphylaxis or hypersensitivity, additional samples for drug concentration and immunogenicity may be collected at or near the onset and the resolution of the event.
²⁵ Blood samples for CH50 (efficacy endpoint) and AH50 will be obtained prior to any study treatment administration (pre-dose).
²⁶ Whole blood samples for DNA extraction (optional) should be collected on day 1 (pre-dose) but can be collected at a later study visit. Patients who had consented to DNA testing in a prior study with Pozelimab and had provided a sample for analysis do not need to provide separate consent/sample for this study.

TABLE 4-3
Schedule of Events for Patients on Intensified Treatment in the OLTP

| Intensified Treatment Period in the OLTP³ | | | | | | | | | | | |
|---|---|---|---|---|---|---|---|---|---|---|---|
| | | | | | | | | | | | EOT |
| RV1 | RV2 | RV3 | RV4 | RV5 | RV6 | RV7 | RV8 | RV9 | RV10 | RV11 | RV12 |
| Week | | | | | | | | | | | |
| 0r | 1r | 2r | 4r | 6r | 8r | 10r | 12r | 16r⁵ | 20r⁵ | 24r⁵ | 28r⁶ |
| Day | | | | | | | | | | | |
| 1r | 8r | 15r | 29r | 43r | 57r | 71r | 85r | 113r | 141r | 169r | 197r |
| Window (day) | | | | | | | | | | | |
| — | ±3 | ±3 | ±3 | ±3 | ±3 | ±3 | ±3 | −7/+3 | −7/+3 | −7/+3 | −7/+3 |

Intensified Treatment:

| Pozelimab 30 mg/kg IV (loading dose)⁴ | x⁷ | | | | | | | | | | | |
|---|---|---|---|---|---|---|---|---|---|---|---|---|
| Pozelimab 400 mg SC Q2W⁴ | x⁷ | | x | x | x | x | x | x | x | x | x⁴ | |
| Cemdisiran 200 mg SC Q4W⁴ | x⁷ | | | x | | x | | x | x | x | x | |
| Injection training/patient instructions | | <----------------------------------------x---------------------------------------------------> | | | | | | | | | | |
| (as needed)⁸ | | | | | | | | | | | | |
| Patient diary⁹ | x | x | x | x | x | x | x | x | x | x | x | x |

| | |
|---|---|
| Antibiotics prophylaxis (recommended)¹⁰ | <----------------------------------------x--------------------------------------------------->> |
| Revaccination against meningococcal | <----------------------------------------x---------------------------------------------------> |
| infection (if needed) | |

Clinical Outcome Assessments:

| FACIT-Fatigue | x | x | x | x | x | x | x | x | x |
|---|---|---|---|---|---|---|---|---|---|
| EORTC-QLQ-C30 | x | x | x | x | x | x | x | x | x |
| TSQM | x | x | x | x | x | x | x | x | x |
| PNH symptom-specific questionnaire | <----------------------------------------x---------------------------------------------------> | | | | | | | | |
| (daily)¹¹ | | | | | | | | | |
| PGIS | x | | x | | x | | x | | x |
| PGIC | | | x | | x | | x | | x |

Safety and Anthropometric:

| | | | | | | | | | | | | |
|---|---|---|---|---|---|---|---|---|---|---|---|---|
| Patient safety card for | x | x | x | x | x | x | x | x | x | x | x | x |
| Neisseria meningitidis¹² | | | | | | | | | | | | |
| Body weight | x | | | x | | x | | x | x | x | x | x |
| Vital signs | x | | x | x | | x | | x | x | x | x | x |
| Physical examination | x | | | x | | | | x | | | | x |
| Electrocardiogram | | | | | | | | | | | | x |
| Adverse events | x | x | x | x | x | x | x | x | x | x | x | x |
| Breakthrough hemolysis assessment¹³ | x | x | x | x | x | x | x | x | x | x | x | x |
| Concomitant meds/treatments | x | x | x | x | x | x | x | x | x | x | x | x |
| Transfusion record update | x | x | x | x | x | x | x | x | x | x | x | x |

Laboratory Testing¹⁴

| | | | | | | | | | | | | |
|---|---|---|---|---|---|---|---|---|---|---|---|---|
| Coagulation panel | x | x | x | x | x | x | x | x | x | x | x | x |
| Chemistry (long panel) including LDH¹⁵ | x | x | x | x | x | x | x | x | x | x | x | x |
| Hematology¹⁶ | x | x | x | x | | x | | x | x | x | x | x |
| Immunoglobulin G | x | | | x | | | | x | | | | |
| Pregnancy test (WOCBP only)¹⁷ | u | | | u | | u | | u | u | u | u | u |
| Urinalysis | x | x | x | x | | x | | x | x | x | x | x |

Pharmacokinetics and Immunogenicity Sampling:

| | | | | | | | | | | |
|---|---|---|---|---|---|---|---|---|---|---|
| Pozelimab drug conc. sample¹⁸ | x | x | x | x | x | x | x | x | x | x |
| Cemdisiran and its metabolite conc. | x | | | x | | x | | | x | |
| sample¹⁹ | | | | | | | | | | |
| Pozelimab immunogenicity sample²⁰ | x | | | | | x | | | | x |
| Cemdisiran immunogenicity sample²⁰ | x | | | | | x | | | | x |
| Total C5 (plasma)¹⁸ | x | x | x | x | x | x | x | x | x | x |

Biomarkers:

| | | | | | | | | | | |
|---|---|---|---|---|---|---|---|---|---|---|
| Free hemoglobin | x | | | x | x | x | x | x | x | x |
| Haptoglobin | x | | | x | | x | | | | x |
| Complement hemolytic assay | x | x | x | x | x | x | x | x | x | x |
| (serum CH50)²¹ | | | | | | | | | | |
| Complement hemolytic assay | x | x | x | x | x | x | x | x | x | x |
| (serum AH50)²¹ | | | | | | | | | | |
| sC5b-9 (plasma) | x | x | x | x | x | x | x | x | x | x |
| PNH erythrocyte cells | x | | | | x | | | x | | x |
| PNH granulocyte cells | x | | | | x | | | x | | x |

1. Visits between week 6r and week 24r may be at the clinical site, or another preferred location, such as patient's home. The location will be dependent on availability of home healthcare visiting professional as well as the preferences of the investigator and patient. In the event of travel restrictions due to a global pandemic, alternative mechanisms such as, but not limited to, telemedicine visits may be implemented to maintain continuity of study conduct.
2. When multiple procedures are performed on the same day, the sequence of procedures is as follows: COAs → ECG → vital signs, physical examination, safety monitoring, lab collection → study drug administration → any pre-specified post-dose sample collection.
³The intensified treatment schedule will be anchored to the day of intensification (i.e., a reset occurs with the day of intensification becoming the day 1r visit and subsequent visits following the schedule of events for intensified treatment). Patients who receive intensified treatment will be considered to have complete the study once they finish the 28-week treatment period with the intensified treatment (i.e., after completing week 28r EOT assessments).
⁴During the intensified treatment period in the OLTP, the dose of Cemdisiran and Pozelimab SC should be given on the day of the corresponding study visit whenever possible. Study treatment administration should always be the last procedure after all blood sample collection and study assessments have been completed unless otherwise specified. If Pozelimab or Cemdisiran cannot be administered on the day of the corresponding study visit, the combination may be administered up to 3 days before or up to 3 days after the planned dosing date provided that the dosing takes place after the corresponding study visit has been completed. For example, the day 29r (week 4r) visit can take place from day 26r to day 32r given the visit window of ±3 days for the week 4r visit. The dose of Pozelimab and Cemdisiran, therefore, can be given from day 26r to day 32r, but only on or after the week 4r visit assessments have been performed. Similarly, the day 113r (week 16r) visit can take place from day 106r to day 116r given the visit window of −7/+3 days for the week 16r visit. The dose of Pozelimab and Cemdisiran can be given from day 110r to day 116r, but only on or after the week 16r visit assessments have been performed. Care must be taken to coordinate dosing for visits where a post-dose sample is collected to measure concentration of Cemdisiran and its metabolites. For patients on intensified treatment in the OLTP, the final SC dose of Cemdisiran is at week 24r and the final SC dose of Pozelimab is at week 26r.
⁵For these visits, the dosing window (±3 days) is narrower than the study visit window (−7/+3 days).
⁶If the patient agrees to continue into the optional OLEP, the EOT visit of the OLTP will correspond to the day 1e visit of the OLEP. Any common assessments will be performed once for both visits.
⁷On day 1r, Pozelimab IV will be given first, with a 30-minute observation period before administration of SC doses. Subsequent Pozelimab SC dose will be administered Q2W and Cemdisiran SC dose will be administered Q4W. The SC injections may either be performed by the site personnel or another healthcare professional at patient's home or preferred location, or be administered by the patient or by a designated person who has successfully completed the injection training.
⁸Injection training will be provided to patients who desire self-injection or injection by a designated person. Site should observe patient self-injection or injection by a designated person and confirm adequacy. Patient instruction materials will be provided.
⁹If study treatment is given by the patient or by a designated person, the patient will complete a diary for recording compliance with study treatment administration. If patient diary is provided to the patient, then it should be reviewed at each clinic visit and data collected into the CRFs. On the final visit, the diary should be collected by the site.
¹⁰Daily oral antibiotic prophylaxis against Neisseria meningitidis is recommended until 52 weeks after discontinuation of study treatment.
¹¹Patients should try to complete the PNH Symptom-Specific Questionnaire at the same time each day whenever possible.
¹²Patient safety card: Site should review the instructions on the safety card with the patient at each visit. Replacement cards may be given to the patient as needed.
¹³Breakthrough hemolysis assessment: If a patient is suspected of having a breakthrough hemolysis event, then, in addition to the required laboratory collection, additional samples for drug concentrations of Pozelimab will be collected unless already noted in the schedule of events for that visit. If the suspected event does not occur at a scheduled visit, then an unscheduled visit should occur with an evaluation of the patient and collection of coagulation, chemistry, and drug concentrations of Pozelimab.
¹⁴Clinical lab samples will be collected prior to any study drug administration (pre-dose) unless otherwise specified. During lab collection, handling and processing, the same methodology will be applied across study visits, as best as possible, to preserve the quality of sample and avoid hemolysis. The coagulation blood sample (tube) must always be collected first, followed by the blood chemistry sample (tube).
¹⁵Serum LDH, CRP, and bilirubin (total and direct) will be assessed as part of the blood chemistry analysis. Blood chemistry sample should be collected before study treatment administration (pre-dose). During lab collection, handling and processing, the same methodology will be applied across study visits, as best as possible, to preserve the quality of sample and avoid hemolysis during sample processing. If the investigator or sponsor suspects that the lab result is not an accurate reflection of the patient's condition, the lab sample should be repeated.
¹⁶Hematology sample should be collected before study treatment administration (pre-dose).
¹⁷Pregnancy test for WOCBP: A urine test will be done at all visits indicated. Any positive urine pregnancy test should be confirmed with a serum pregnancy test.
¹⁸On day 1r, obtain blood sample for Pozelimab concentration and total C5 prior to IV administration of Pozelimab and also within 15 minutes after the end of the IV infusion. At subsequent timepoints, blood samples for Pozelimab drug concentration analysis and total C5 analysis will be obtained prior to any study treatment administration (pre-dose).
¹⁹Blood samples for Cemdisiran drug concentration analysis and concentrations of its metabolites will be collected on the specified days prior to any study treatment administration (pre-dose) and 2 to 6 hours post-Cemdisiran administration. The post-dose sample should be carefully coordinated with the dosing of Cemdisiran and may be collected at the clinic or by a visiting health care professional.
²⁰Blood samples for immunogenicity will be collected on the specified days prior to any study treatment administration (pre-dose). At the visits where immunogenicity samples are to be taken, the sample should be collected with the sample for drug concentration. In the event of suspected treatment-related SAEs, such as anaphylaxis or hypersensitivity, additional samples for drug concentration and immunogenicity may be collected at or near the onset and the resolution of the event.
²¹Blood samples for CH50 (efficacy endpoint) and AH50 will be obtained prior to any study treatment administration (pre-dose).

TABLE 4-4
Schedule of Events (Optional Open-Label Extension Period)

| Optional Open-Label Extension Period | | | | | | |
|---|---|---|---|---|---|---|
| OLEP-1³ | OLEP-2⁵ | OLEP-3⁵ | OLEP-4⁵ | OLEP-5⁵ | OLEP-6⁵ | OLEP-7 |
| Week | | | | | | |
| 0e | 8e | 16e | 24e | 32e | 40e | 52e |
| Day | | | | | | |
| 1e | 57e | 113e | 169e | 225e | 281e | 365e |
| Window (day) | | | | | | |
| — | −7/+3 | −7/+3 | −7/+3 | −7/+3 | −7/+3 | −7/+3 |

Treatment⁴:

| Pozelimab 400 mg SC Q2W or Q4W⁵ | x | x | x | x | x | x | x |
|---|---|---|---|---|---|---|---|
| Cemdisiran 200 mg SC Q4W⁵ | x | x | x | x | x | x | x |
| Injection training/patient instructions | <--------------------------------------------x---------------------------------------------------> | | | | | | |
| (as needed)⁶ | | | | | | | |
| Patient diary⁷ | x | x | x | x | x | x | x |

| | |
|---|---|
| Antibiotics prophylaxis (recommended)⁸ | <--------------------------------------------x---------------------------------------------------> |
| Revaccination against meningococcal | <--------------------------------------------x---------------------------------------------------> |
| infection (if needed) | |

Clinical Outcome Assessments:

| | | | |
|---|---|---|---|
| FACIT-Fatigue | x | x | x |
| EORTC-QLQ-C30 | x | x | x |
| PGIS | x | x | x |
| PGIC | x | x | x |

Safety and Anthropometric:

| | | | | | | | |
|---|---|---|---|---|---|---|---|
| Patient safety card for | x | x | x | x | x | x | x |
| Neisseria meningitidis ⁹ | | | | | | | |
| Body weight | x | x | x | x | x | x | x |
| Vital signs | x | x | x | x | x | x | x |
| Physical examination | x | | x | | x | | x |
| Electrocardiogram | x | | | | | | x |
| Adverse events | x | x | x | x | x | x | x |
| Breakthrough hemolysis | x | x | x | x | x | x | x |
| assessment¹⁰ | | | | | | | |
| Concomitant meds/treatments | x | x | x | x | x | x | x |
| Transfusion record update | x | x | x | x | x | x | x |

Laboratory Testing¹¹:

| | | | | | | | |
|---|---|---|---|---|---|---|---|
| Coagulation panel | x | x | x | x | x | x | x |
| Chemistry (long panel) | x | x | x | x | x | x | x |
| including LDH¹² | | | | | | | |
| Hematology¹³ | x | x | x | x | x | x | x |
| Pregnancy test (WOCBP only)¹⁴ | u | u | u | u | u | u | u |
| Urinalysis | x | x | x | x | x | x | x |

Pharmacokinetics and Immunogenicity:

| | | | |
|---|---|---|---|
| Pozelimab conc. sample¹⁵ | x | x | x |
| Cemdisiran and its metabolite | x | x | x |
| conc. samples¹⁶ (pre-dose and | | | |
| 2 to 6 hours post-dose) | | | |
| Pozelimab immunogenicity sample¹⁷ | x | x | x |
| Cemdisiran immunogenicity sample¹⁷ | x | x | x |
| Total C5 (plasma)¹⁵ | x | x | x |

Biomarkers:

| | | | | | |
|---|---|---|---|---|---|
| Free hemoglobin | x | | | | x |
| Haptoglobin | x | | | | x |
| Complement hemolytic assay | x | x | | x | x |
| (serum CH50)¹⁸ | | | | | |
| Complement hemolytic assay | x | x | | x | x |
| (serum AH50)¹⁸ | | | | | |
| sC5b-9 (plasma) | x | x | | x | x |
| PNH erythrocyte cells | x | | x | | x |
| PNH granulocyte cells | x | | x | | x |

Optional research:

| | | |
|---|---|---|
| Serum and plasma for FBR | x | x |
| (optional) | | |
| Whole blood RNA sample for | x | x |
| genomics research (optional) | | |

1. Visits may be at the clinical site or another preferred location, such as the patient's home. The location will depend on availability of home healthcare visiting professional as well as the preferences of the investigator and patient. In the event of travel restrictions due to a global pandemic, alternative mechanisms such as but not limited to telemedicine visits may be implemented to maintain continuity of study conduct.
2. When multiple procedures are performed on the same day, the sequence of procedures is as follows: COAs → ECG → vital signs, physical examination, safety monitoring, lab collection → study drug administration → any pre-specified post-dose sample collection.
³Day 1e of OLEP should be scheduled on the same day as week 28 (or week 28r for patients on intensified treatment) of the OLTP, and any common assessments will be performed once for both the OLTP and OLEP visits.
⁴For patients who did not receive intensified treatment during OLTP: At any time during the OLEP, patients who meet pre-specified criteria will receive intensified treatment of a Pozelimab 30 mg/kg IV loading dose followed 30 minutes later by the initiation of Pozelimab 400 mg SC Q2W and Cemdisiran 200 mg SC Q4W. Patients should be observed for 30 minutes in the interim between the IV and SC doses. Patients will continue their visit schedule at the next OLEP visit.
⁵During the OLEP, the dose of Cemdisiran and Pozelimab SC should be given on the day of the corresponding study visit whenever possible. Study treatment administration should always be the last procedure after all blood sample collection and study assessments have been completed unless otherwise specified:
If Pozelimab or Cemdisiran cannot be administered on the day of the corresponding study visit, the combination may be administered up to 3 days before or up to 3 days after the planned dosing date, provided that the combination dosing takes place after the corresponding study visit has been completed. For patients receiving Pozelimab 400 mg SC Q2W + Cemdisiran 200 mg SC Q4W, the dosing window (±3 days) is narrower than the visit window (−7/+3 days).
If Pozelimab or Cemdisiran cannot be administered on the day of the corresponding study visit, the combination may be administered up to 7 days before or 7 days after the planned dosing date, provided that the combination dosing takes place after the corresponding study visit has been completed. For patients receiving Pozelimab 400 mg SC Q4W + Cemdisiran 200 mg SC Q4W, the visit window (−7/+3 days) is narrower than the dosing window (±7 days).
Care must be taken to coordinate dosing for visits where a post-dose sample is collected to measure concentration of Cemdisiran and its metabolites. For patients whose treatment is not intensified during the OLEP, the last doses of Cemdisiran and Pozelimab are administered at week 52e. For patients whose treatment was intensified during the OLEP, the last doses of study treatment will be determined based on the time of treatment intensification.
⁶Injection training will be provided to patients who desire self-injection or injection by a designated person. Site should observe patient self-injection or injection by a designated person and confirm adequacy. Patient instruction materials will be provided.
⁷If study treatment is given by the patient or by a designated person, the patient will complete a diary for recording compliance with study treatment administration. If patient diary is provided to the patient, then it should be reviewed at each clinic visit and data collected into the CRFs. On the final visit, the diary should be collected by the site.
⁸Daily oral antibiotic prophylaxis against Neisseria meningitidis is recommended until 52 weeks after discontinuation of study treatment.
⁹ Patient safety card: Site should review the instructions on the safety card with the patient at each visit. Replacement cards may be given to the patient as needed.
¹⁰Breakthrough hemolysis assessment: If a patient is suspected of having a breakthrough hemolysis event, then in addition to the required laboratory collection, additional samples for drug concentrations of Pozelimab will be collected unless already noted in the schedule of events for that visit. If the suspected event does not occur at a scheduled visit, then an unscheduled visit should occur with an evaluation of the patient and collection of coagulation, chemistry, and drug concentration of Pozelimab.
¹¹Clinical lab samples will be collected prior to any study drug administration (pre-dose) unless otherwise specified. During lab collection, handling and processing, the same methodology will be applied across study visits, as best as possible, to preserve the quality of sample and avoid hemolysis. The coagulation blood sample (tube) must always be collected first, followed by the blood chemistry sample (tube).
¹²Serum LDH, CRP, and bilirubin (total and direct) will be assessed as part of the blood chemistry analysis. Blood chemistry sample should be collected before study treatment administration (pre-dose). During lab collection, handling and processing, the same methodology will be applied across study visits, as best as possible, to preserve the quality of sample and avoid hemolysis during sample processing. If the investigator or sponsor suspects that the lab result is not an accurate reflection of the patient's condition, the lab sample should be repeated.
¹³Hematology sample should be collected before study treatment administration (pre-dose).
¹⁴Pregnancy test for WOCBP: A urine test will be done at all visits indicated. Any positive urine pregnancy test should be confirmed with a serum pregnancy test.
¹⁵Blood samples for Pozelimab concentration analysis and total C5 analysis will be obtained on the specified days prior to any study treatment administration (pre-dose).
¹⁶Blood samples for Cemdisiran concentration analysis and concentrations of its metabolites will be collected on the specified days prior to any study treatment administration (pre-dose) and 2 to 6 hours post Cemdisiran administration. The post-dose sample should be carefully coordinated with the dosing of Cemdisiran and may be collected at the clinic or by a visiting health care professional.
¹⁷Blood samples for immunogenicity will be collected on the specified days prior to any study treatment administration (pre-dose). At the visits where immunogenicity samples are to be taken, the sample should be collected with the drug concentration sample. In the event of suspected treatment-related SAEs, such as anaphylaxis or hypersensitivity, additional drug concentration and immunogenicity samples may be collected at or near the onset and the resolution of the event.
¹⁸Blood samples for CH50 (efficacy endpoint) and AH50 will be obtained prior to any study treatment administration (pre-dose).

TABLE 4-5
Schedule of Events (Post-Treatment Safety Follow-Up Period)

| Patients in the OLTP who | | | | | | |
|---|---|---|---|---|---|---|
| discontinue study treatment will | | | | | | |
| be asked to remain in the study | | | | | | |
| until week 28 EOT (or week 28r | | | | | | |
| for patients who restarted on | | | | | | |
| intensified treatment) and follow | Post-Treatment Safety Follow-Up Period | | | | | |
| the original schedule of events as | Patients who completed | | | | | |
| applicable. After the week 28 | week 28 (with last doses | | | | | |
| EOT visit, their entry point into | administered at week 24 | | | | | |
| the safety follow-up schedule | or week 26, or week | | | | | |
| will depend on the number of | 26r) who choose not to | | | | | |
| weeks that have elapsed since | continue treatment in | | | | | |
| their last dose (i.e., a patient who | the OLEP, patients who | | | | | |
| is 20 weeks after their final dose | complete the OLEP, and | | | | | |
| of study treatment at EOT will | patients who | | | | | |
| enter into the safety follow-up | permanently discontinue | | | | | |
| period at visit FU-4 [26 weeks | treatment during the | | | | | |
| after last dose]) | OLEP will start here: | | | | Phone visit | Phone visit |
| Study Procedure | FU-1 | FU-2 | FU-3 | FU-4 | FU-5 | FU-6 |
| Week (after last dose of study | 8 | 12 | 16 | 26 | 38 | 52 |
| drug) | | | | | | |
| Day (after last dose of study | 57 | 85 | 113 | 183 | 267 | 365 |
| drug) | | | | | | |
| Window (day) | ±10 | ±10 | ±10 | ±10 | ±10 | ±10 |

Safety Assessments

| Patient safety card for Neisseria | <---------------------------------------------x -----------------------------------------> | | | | | |
|---|---|---|---|---|---|---|
| meningitidis¹ | | | | | | |
| Antibiotics prophylaxis | <---------------------------------------------x -----------------------------------------> | | | | | |
| (recommended)² | | | | | | |
| Vital signs | x | x | x | x | | |
| Physical examination | | x | | x | | |
| Concomitant meds and | x | x | x | x | x | x |
| procedures | | | | | | |
| Adverse event reporting | x | x | x | x | x | x |
| Pregnancy reporting | x | x | x | x | x | x |

Laboratory Testing

| | | | | |
|---|---|---|---|---|
| Hematology | x | x | x | x |
| Blood chemistry | x | x | x | x |

¹Patient safety card: Site should review the instructions on the safety card with the patient at each visit. Replacement cards may be given to the patient as needed.
²Daily oral antibiotic prophylaxis against N. meningitidis is recommended until 52 weeks after discontinuation of study treatment.

### Intensified Treatment

Patients will be given intensified treatment if they meet both of the following criteria:
Breakthrough hemolysis that is not due to a complement activating condition (i.e., intercurrent infection)*
For LDH increase >2×ULN due to a complement activating condition
Inadequate LDH response (i.e., LDH>1.5×ULN) that is sustained (i.e., on 2 consecutive measurements spanning at least 2 weeks)

Patients' treatment will be intensified depending on their assigned treatment group as outlined below. An assessment of the patient's weight should be performed on the day of the IV Pozelimab load in order to calculate the appropriate dose.
Patients randomized to Pozelimab 400 mg SC Q4W and Cemdisiran 200 mg SC Q4W will receive a single administration of Pozelimab 30 mg/kg IV on the day of intensification+an intensified Pozelimab regimen of 400 mg Q2W along with Cemdisiran 200 mg Q4W starting on the day of intensification. The patients will be treated with the intensified SC dose regimen for additional 28 weeks to complete the study, with the last doses of the intensified treatment at week 24r (for Cemdisiran) and week 26r (for Pozelimab);
Patients randomized to Pozelimab 400 mg SC Q2W and Cemdisiran 200 mg SC Q4W will receive a single administration of Pozelimab 30 mg/kg IV on the day of intensification+a re-initiation of the assigned combination regimen of Pozelimab 400 mg SC Q2W and Cemdisiran 200 mg SC Q4W starting on the day of intensification. The patients will continue the same SC dose regimen for additional 28 weeks to complete the study, with the last doses of the intensified treatment at week 24r (for Cemdisiran) and week 26r (for Pozelimab).

Patients who undergo treatment intensification may require unscheduled visit(s) prior to intensification. During the OLTP, patients whose treatment is intensified should follow Table 4-3 (Schedule of Events for Patients on Intensified Treatment in the OLTP), with the day of intensification anchored to day 1r (RV1). Patients who restarted on an intensified treatment will be considered to have completed the OLTP once they receive 28 weeks of the intensified treatment and complete Week 28r assessments.

During the OLEP, patients who are not on intensified treatment who meet criteria for treatment intensification will receive a single administration of Pozelimab 30 mg/kg IV based on their current weight on the day of intensification+an intensified regimen of Pozelimab 400 mg Q2W along with Cemdisiran 200 mg Q4W starting on the day of intensification and for the remainder of the OLEP. Patients will continue their visit schedule at the next OLEP visit.

Patients who experience breakthrough hemolysis that is not due to a complement activating condition and meet criteria for treatment intensification are eligible to receive intensification of Pozelimab only once (whether during the main treatment period or the OLEP), beyond which no further intensification will be permitted.

Note: In the event of an LDH increase >2×ULN due to an acute complement activating condition during the OLTP or OLEP, an IV bolus of Pozelimab 30 mg/kg IV may be given. An assessment of the patient's weight should be performed on the day of the IV Pozelimab load in order to calculate the appropriate dose. This is not considered treatment intensification. No other changes will be made to the study treatment regimen (i.e., the regular dose and frequency of Pozelimab and Cemdisiran will proceed unchanged). Patients will continue onto the next visit of their current visit schedule.
Note: The IV dose should be administered first. The SC doses should be given at least 30 minutes after completion of the IV administration.

### Primary Objective

The primary objective of the study is to evaluate the safety and tolerability of 2 dosing regimens of Pozelimab and Cemdisiran combination therapy during the open-label treatment period (OLTP).

### Secondary Objectives

The secondary objectives of the study are:
To evaluate the effect of the combination treatment on the following parameters of intravascular hemolysis: LDH control, breakthrough hemolysis, and inhibition of total complement hemolysis activity (CH50);
To evaluate the effect of the combination treatment on hemoglobin levels;
To evaluate the effect of the combination treatment on RBC transfusion requirements;
To evaluate the effect of the combination treatment on COAs measuring fatigue and health related quality of life;
To assess the concentrations of total Pozelimab in serum and total C5 and Cemdisiran in plasma;
To assess immunogenicity to Pozelimab and Cemdisiran;
To evaluate the long-term safety and efficacy of Pozelimab and Cemdisiran in an optional open-label extension period (OLEP);
To assess safety after treatment intensification with Pozelimab and Cemdisiran.

### Exploratory Objectives

The exploratory objectives of the study are:
To explore the effect of the combination treatment on clinical thrombosis events;
To explore the effect of the combination treatment on renal function and renal injury biomarkers;
To explore the effect of the combination treatment on complement activation and intravascular hemolysis relevant to PNH and other related diseases;
To explore the effect of the combination treatment on PNH clone size;
To evaluate the effect of the combination treatment on treatment satisfaction;
To explore the effect of the combination treatment on a novel COA measuring PNH-specific symptoms;
To study the combination treatment mechanism of action (including relationship to safety and efficacy), complement pathway biology, PNH and related complement mediated diseases;
To explore the effect of the combination treatment on PNH symptoms;
To explore potential differences in genotype and gene expression that may influence efficacy and safety of the combination treatment for further understanding of C5, PNH, or other conditions associated with complement-mediated injury (for patients who consent to participate in a genomics sub-study);
To explore safety and efficacy after dose intensification with Pozelimab and Cemdisiran;
To explore the long-term effects of the combination treatment on clinical and PD assessments in an optional OLEP.

### Primary Endpoint

The primary endpoint is the incidence and severity of TEAEs through week 28 of the OLTP.

### Secondary Endpoints

The secondary endpoints for the OLTP are:
Percent change of LDH from pre-treatment (defined as mean of LDH values at day −7 and day 1 [prior to combination dosing]) to end-of-treatment period (defined as mean of LDH values at week 24 through week 28);
Maintenance of adequate control of hemolysis, defined as LDH≤1.5×ULN from post-baseline (on day 1) through week 28 and from week 4 through week 28, inclusive;
Adequate control of hemolysis (defined as LDH≤1.5×ULN) at each visit from post-baseline (on day 1) through week 28, inclusive;
Normalization of LDH at each visit, defined as LDH≤1.0×ULN from post-baseline (on day 1) through week 28, inclusive;
Area under the curve (AUC) of LDH over time from baseline though week 28 and from week 4 through week 28, inclusive;
Breakthrough hemolysis (as defined herein) from baseline through week 28;
Hemoglobin stabilization (defined as patients who do not receive RBC transfusion and have no decrease in hemoglobin levels of >2 g/dL) from baseline through week 28;
Change in hemoglobin levels from baseline to week 28;
Transfusion avoidance (defined as not requiring a RBC transfusion as per protocol algorithm) from baseline to week 28;
Rate and number of units of RBCs transfused from baseline to week 28;
Change in CH50 from baseline to week 28;
Change in fatigue as measured by Functional Assessment of Chronic Illness Therapy-Fatigue (FACIT-Fatigue) scale from baseline to week 28;
Change from baseline to week 28 in global health status/quality of life scale (GHS/QoL) and physical function (PF) scores on the European Organization for Research and Treatment of Cancer: Quality-of-Life Questionnaire core 30 items (EORTC QLQ-C30);
Concentrations of total Pozelimab in serum and Cemdisiran in plasma, assessed throughout the study;
Change from baseline in concentration of total C5 assessed throughout the study;
Assessment of immunogenicity to Pozelimab and Cemdisiran as determined by the incidence, titer, and clinical impact of treatment-emergent anti-drug antibody (ADA) responses over time;
Incidence and severity of TEAEs for patients who received treatment intensification through week 28r.

### Exploratory Endpoints

The exploratory endpoints for the OLTP are:
Treatment intensification throughout the study;
Incidence of major adverse vascular events (MAVE) from baseline to week 28. MAVE includes thrombophlebitis/deep vein thrombosis, pulmonary embolus, myocardial infarction, unstable angina, renal vein or artery thrombosis, acute peripheral vascular occlusion, hepatic vein thrombosis, portal vein thrombosis mesenteric/visceral vein thrombosis or infarction, mesenteric/visceral arterial thrombosis or infarction, transient ischemic attack, cerebral arterial occlusion/cerebrovascular accident, cerebral venous occlusion, gangrene (nontraumatic; non-diabetic), and amputation (nontraumatic; non-diabetic);
Change in renal function as measured by estimated glomerular filtration rate (eGFR) from baseline to week 28;
Percent change in free hemoglobin from baseline to week 28;
Change in bilirubin from baseline to week 28;
Change in reticulocyte count from baseline to week 28;
Change and percent change in the alternative pathway hemolytic activity assay (AH50) from baseline to week 28;
Proportion of PNH erythrocytes and granulocytes from baseline to week 28
Change from baseline to week 28 in functional scale scores (Role functioning, Emotional Functioning, Cognitive Functioning and Social Functioning) and symptom scale scores (Fatigue, Nausea and vomiting, Pain, Dyspnoea, Insomnia, Appetite loss, Constipation, Diarrhoea) of the EORTC QLQ-C30;
Stability in global health status, functioning and symptoms as measured by the EORTC QLQ-C30 from baseline to week 28;
Comparison of treatment satisfaction (as assessed by the Treatment Satisfaction Questionnaire for Medication [TSQM]) at baseline vs. treatment at week 28;
Change in Patient Global Impression of Severity (PGIS) from baseline to week 28, including questions on PNH symptoms, impacts and fatigue;
Patient Global Impression of Change (PGIC) at week 28, including questions on PNH symptoms, impacts and fatigue;
Change in PNH symptoms as measured by the PNH symptom-specific questionnaire from baseline to week 28;
Exploratory endpoints related to the analyses for those patients receiving dose intensification and patients participating in the OLEP will be provided in a statistical analysis plan (SAP).

### Breakthrough Hemolysis

Patients who experience breakthrough hemolysis may receive intensified treatment as described herein. Breakthrough hemolysis is defined as an increase in LDH with concomitant signs or symptoms associated with hemolysis:
An increase in LDH occurs when:
   LDH≥2×ULN if pre-treatment LDH is ≤1.5×ULN; or
   LDH≥2×ULN after initial achievement of LDH≤1.5×ULN if pre-treatment LDH is >1.5×ULN

The signs or symptoms should correspond to those known to be associated with intravascular hemolysis due to PNH, limited to the following: new onset or worsening fatigue, headache, dyspnea, hemoglobinuria, abdominal pain, scleral icterus, erectile dysfunction, chest pain, confusion, dysphagia, new thrombotic event, anemia including hemoglobin value significantly lower (i.e., ≥2 g/dL decrease) as compared to patient's known baseline hemoglobin values.

### Efficacy Variables

Efficacy in this study is evaluated by the following laboratory assessments (Laboratory Variables for the Assessment of Efficacy):
LDH (serum): LDH as a measure of intravascular hemolysis allows for an objective and precise means to gauge whether the control of intravascular hemolysis is sustained when the patients are switched to Pozelimab and Cemdisiran combination treatment;
Hemoglobin: Hemolytic anemia is a hallmark of PNH;
CH50: The CH50 assay will be used to confirm complete inhibition of complement activity has been achieved throughout the dosing interval in patients with PNH.

These laboratory variables are relevant to the characterization and disease mechanisms of PNH (Brodsky, Paroxysmal nocturnal hemoglobinuria. Blood 2014; 124(18):2804-11).

Hemolytic anemia is a clinical manifestation of PNH, and patients often require blood transfusion for symptomatic management. The frequency of blood transfusion has been used in other studies of PNH to assess efficacy (Hillmen et al., The complement inhibitor Eculizumab in paroxysmal nocturnal hemoglobinuria. N Engl J Med 2006; 355(12):1233-43) (Röth et al., Ravulizumab (ALXN1210) in patients with paroxysmal nocturnal hemoglobinuria: results of 2 phase 1b/2 studies. Blood Adv 2018; 2(17):2176-85).

Clinical Outcome Assessments (COAs) include the following:
FACIT-Fatigue (Functional Assessment of Chronic Illness Therapy-Fatigue (FACIT-Fatigue));
EORTC-QLQ-C30 (European Organization for the Research and Treatment of Cancer: Quality of Life of Cancer Patients Questionnaire-30 (EORTC-QLQ-C30));
TSQM (Treatment Satisfaction Questionnaire for Medication);
PNH Symptom-Specific Questionnaire;
PGIS (Patient Global Impression of Severity);
PGIC (Global Impression of Change).

Patients complete the Functional Assessment of Chronic Illness Therapy (FACIT)-Fatigue scale and the European Organization for Research and Treatment of Cancer: Physical function assessments and QoL Questionnaire GHS/QoL. FACIT-Fatigue scores range from 0 to 52, with higher scores indicating less fatigue. EORTCQLQ-C30 scores range from 0 to 100; a high score for a functional scale represents a high/healthy level of functioning and a high score for the GHS/QoL represents a high QoL. These assessments are completed at baseline and intermittently throughout the OLTP. Higher scores indicate less fatigue or a better level of functioning or GHS/QoL. In this study, all baseline values and assessments are carried out before patients received the first dose of their assigned combination therapy. Pre-treatment baseline values are given for a previous phase 2 trial where all patients receive open-label Pozelimab monotherapy (see NCT03946748).

### Results (22 Pts, Median Duration 57 Days)

At the time of this analysis, all 22 randomized patients were ongoing in the study (median duration of 57 days), including one who had completed the OLTP and was continuing in the OLEP, and no patient required treatment intensification. At study baseline, patients were well controlled on Pozelimab monotherapy. Patient baseline demographics and characteristics from this study are summarized in Table 4-1.

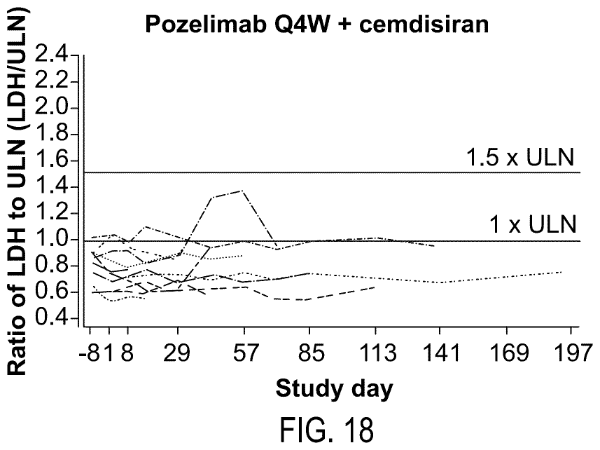

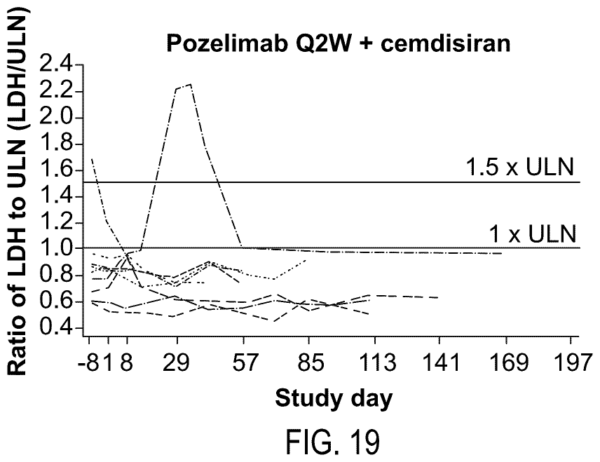

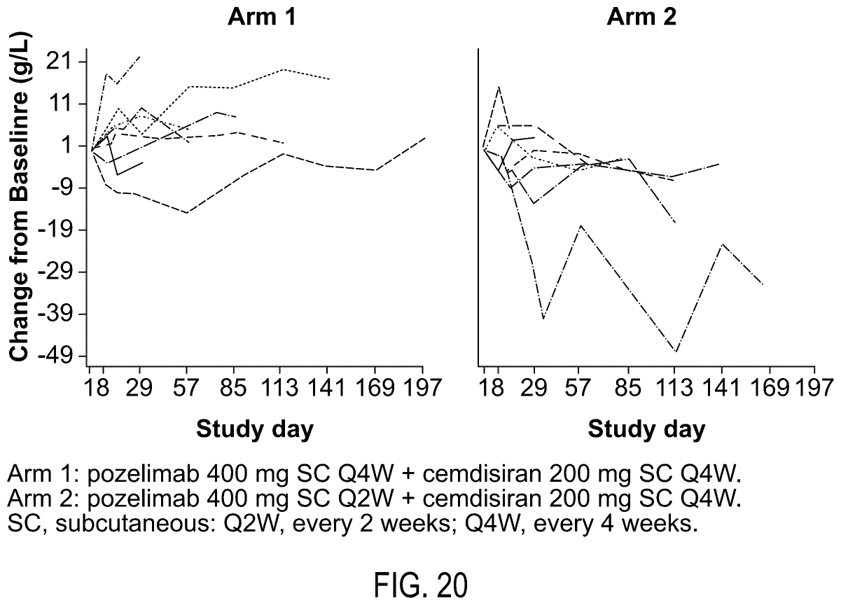

Efficacy outcomes: Twenty-one patients (95.5%) maintained adequate control of hemolysis (LDH; ≤1.5×ULN) up until the data cut-off (FIG. 18 and FIG. 19). In addition, most patients maintained an LDH value below 1.0×ULN for most of the observation period. Most (90.9%) of the patients (n=20) met the criteria for hemoglobin stabilization (did not receive a red blood cell transfusion, no decrease in hemoglobin level ≥2 g/dL). Two patients received blood transfusions and therefore did not meet the criteria for hemoglobin stabilization. Both patients recovered and continued to receive the combination therapy during the course of the study. One patient in arm 1 received a blood transfusion only. Another patient in arm 2 experienced an episode of moderate non-serious breakthrough hemolysis (in association with a chlamydia infection) and a decrease in hemoglobin level ≥2 g/dL and also received a blood transfusion. At the time of this analysis, 90.9% (20 of 22) patients met the criteria for hemoglobin stabilization (i.e., did not receive a red blood cell transfusion and had no decrease in hemoglobin ≥2 g/dL) (FIG. 20). CH50, a measure of total complement hemolysis activity, remained fully suppressed at all post-baseline time-points measured, including during the aforementioned breakthrough hemolysis event.

Treatment-emergent adverse events (TEAEs): There were no serious TEAEs or TEAEs leading to study drug discontinuation in any patient from either treatment group. Importantly, there were no meningococcal infections or TEAEs leading to death in this study. Six patients (27.3%; three from each treatment arm) experienced a total of 12 TEAEs. One patient (8.3%) in arm 1 experienced one adverse event of special interest, a mild injection-site reaction characterized by stinging lasting 30 minutes. All TEAEs were of mild-to-moderate intensity, except for a single severe TEAE of anemia occurring in one patient from the Pozelimab Q2W+Cemdisiran treatment group (arm 2). This same patient had previously experienced a moderate non-serious TEAE of breakthrough hemolysis, which was not considered related to the study treatment by the investigator.

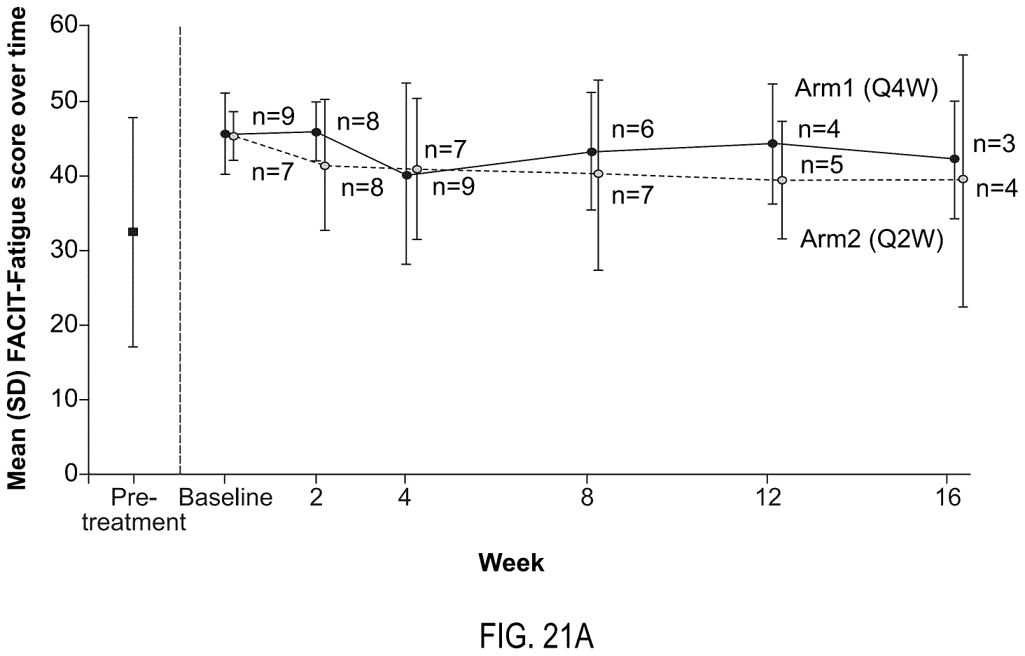

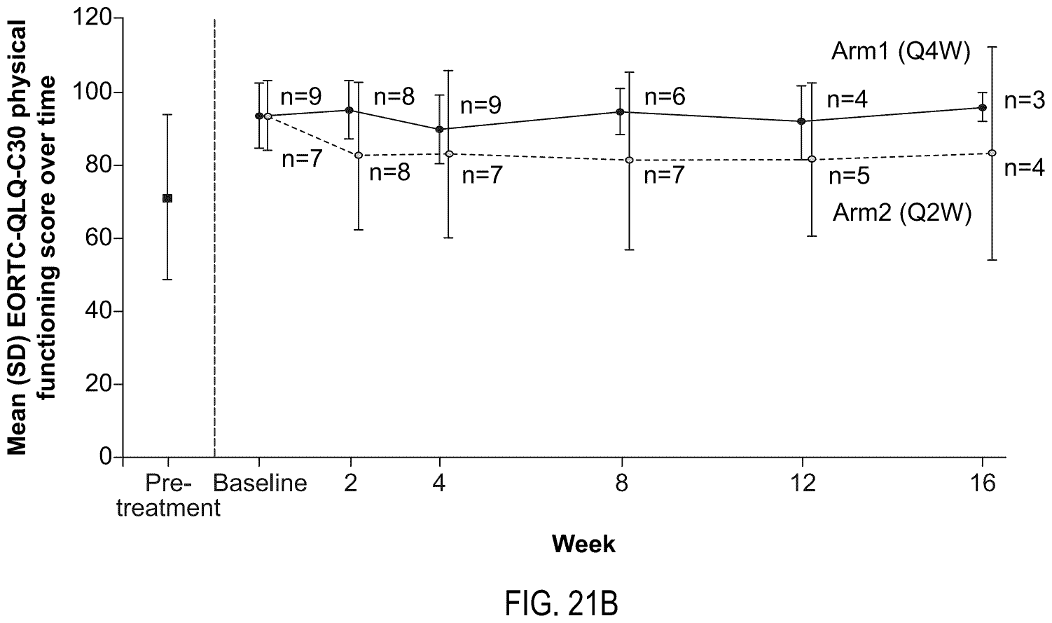

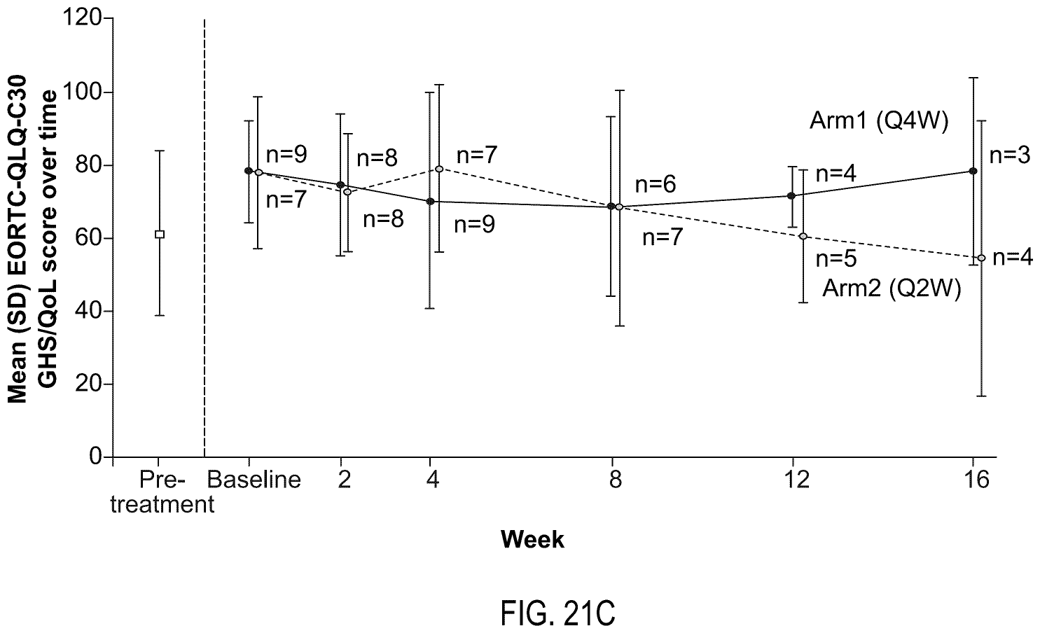

Patient-reported outcomes: Before receiving Pozelimab monotherapy in the previous phase 2 trial (NCT03946748), mean (standard deviation [SD]) pre-treatment values were 32.3 (15.2) for FACIT-Fatigue scores, 70.9 (22.5) for physical functioning scores, and 60.6 (22.4) for GHS/QoL scores. For this study, baseline values were representative of the Pozelimab monotherapy that the patients were receiving prior to transitioning to the combination therapy. At study baseline, when patients transitioned from previous Pozelimab monotherapy, mean (SD) FACIT-Fatigue scores were 45.4 (5.5) and 45.0 (3.4) for arm 1 and arm 2, respectively (FIG. 21A). Over weeks 2-16 of combination treatment, the mean FACIT-Fatigue scores were 40.1-45.6 for arm 1 and 39.2-41.4 for arm 2. At baseline, the mean physical functioning score was 93.3 for both treatment arms (SD: 8.8 for arm 1 and 9.4 for arm 2; FIG. 21B). Over weeks 2-16 of combination treatment, mean physical functioning scores were 89.6-95.6 and 81.0-83.3 for arm 1 and arm 2, respectively. Mean (SD) scores at baseline reflect improved GHS/QoL and were similar for both treatment arms: 77.8 (14.4) and 77.4 (20.8) for arm 1 and arm 2, respectively (FIG. 21C). Over weeks 2-16 of combination treatment, mean GHS/QoL scores were 68.1-77.8 for arm 1 and 54.2-78.6 for arm 2. Patient-reported outcomes are summarized in Table 4-6.

TABLE 4-6
Patient-Reported Outcomes

| | | | EORTC-QLQ-C30 GHS/QoL | | EORTC-QLQ-C30 physical | |
|---|---|---|---|---|---|---|
| | FACIT-Fatigue over time^{b} | | over time^{c} | | functioning over time^{c} | |
| | Pozelimab | Pozelimab | Pozelimab | Pozelimab | Pozelimab | Pozelimab |
| | Q4W + | Q2W + | Q4W + | Q2W + | Q4W + | Q2W + |
| | Cemdisiran | Cemdisiran | Cemdisiran | Cemdisiran | Cemdisiran | Cemdisiran |
| Visit | (n = 12) | (n = 10) | (n = 12) | (n = 10) | (n = 12) | (n = 10) |
| Pre- | 22 | | 22 | | 22 | |
| treatment, n | | | | | | |
| Mean | 32.3 (15.2) | | 60.6 (22.4) | | 70.9 (22.5) | |
| [SD]^{a} | | | | | | |
| Baseline, n | 9 | 7 | 9 | 7 | 9 | 7 |
| Mean | 45.4 (5.5) | 45.0 (3.4) | 77.8 (14.4) | 77.4 (20.8) | 93.3 (8.8) | 93.3 (9.4) |
| (SD) | | | | | | |
| Week 2, n | 8 | 8 | 8 | 8 | 8 | 8 |
| Mean | 45.6 (4.0) | 41.4 (8.8) | 74.0 (19.6) | 71.9 (16.0) | 95.0 (7.8) | 82.5 (20.1) |
| (SD) | | | | | | |
| Week 4, n | 9 | 7 | 9 | 7 | 9 | 7 |
| Mean | 40.1 (12.0) | 40.7 (9.6) | 69.4 (29.5) | 78.6 (23.0) | 89.6 (9.5) | 82.9 (22.7) |
| (SD) | | | | | | |
| Week 8, n | 6 | 7 | 6 | 7 | 6 | 7 |
| Mean | 43.0 (7.9) | 40.0 (12.7) | 68.1 (24.4) | 67.9 (32.1) | 94.4 (6.6) | 81.0 (24.2) |
| (SD) | | | | | | |
| Week 12, n | 4 | 5 | 4 | 5 | 4 | 5 |
| Mean | 44.0 (8.0) | 39.2 (7.9) | 70.8 (8.3) | 60.0 (18.1) | 91.7 (10.0) | 81.3 (20.8) |
| (SD) | | | | | | |
| Week 16, n | 3 | 4 | 3 | 4 | 3 | 4 |
| Mean | 42.0 (7.8) | 39.3 (16.9) | 77.8 (25.5) | 54.2 (37.6) | 95.6 (3.8) | 83.3 (29.1) |
| (SD) | | | | | | |

^{a}Pre-treatment baseline values are given for the previous phase 2 trial where all patients received open-label Pozelimab monotherapy (NCT03946748).
^{b}FACIT-Fatigue: scores range from 0 to 52, with higher scores indicating less fatigue.
^{c}EORTC QLQ-C30: scores range from 0 to 100; a high score for a functional scale represents a high/healthy level of functioning, a high score for the GHS/QoL represents a high QoL.

### Conclusions

The combination of Pozelimab and Cemdisiran was generally well tolerated in patients with PNH who transitioned from Pozelimab monotherapy (NCT04162470), regardless of the treatment arm. Over 90% of patients maintained adequate control of hemolysis and achieved hemoglobin stabilization. Furthermore, most patients maintained normalization of their LDH during the observation period. Accordingly, this combination therapy demonstrates significant efficacy in treating patients with PNH who transitioned from Pozelimab monotherapy.

Additionally, PNH patients who transitioned from Pozelimab monotherapy experienced meaningful improvements in pre-treatment fatigue (>5 point improvement in FACIT-Fatigue), and improvements in physical functioning, and GHS/QoL. Improvements in these scores were maintained by the combination treatment through to Week 16, particularly with the Pozelimab Q4W and Cemdisiran dose regimen. Despite the limited sample size, this evidence shows that Pozelimab and Cemdisiran combination therapy, especially the Q4W regimen, maintained improvements in patient fatigue, physical functioning, and GHS/QoL.

### Results (Week 28)

Prior to receiving Pozelimab monotherapy, mean (standard deviation [SD]) pre-treatment values were 32.3 (15.2) for FACIT-Fatigue scores (Table 4-13), 60.6 (22.4) for GHS/QoL scores (Table 4-23), and 70.9 (22.5) for physical functioning scores (Table 4-33; and Table 4-7). For the current trial, baseline values were representative of the effect of Pozelimab monotherapy that the patients were receiving prior to transitioning to the combination therapy. The mean (SD) FACIT-Fatigue score at baseline was 45.4 (5.6) for arm 1 (Pozelimab q4w and Cemdisiran q4w) and 45.6 (3.6) for arm 2 (Pozelimab q2w and Cemdisiran q4w) (Table 4-14; and Table 4-7). Over Weeks 2-28, the mean FACIT-Fatigue scores were 40.3-45.2 for arm 1 and 36.5-42.9 for arm 2 (Table 4-14-Table 4-22). The mean (SD) physical functioning score at baseline was 93.3 (8.8) for arm 1 and 94.2 (9.0) for arm 2 (Table 4-34). Over Weeks 2-28, the mean physical functioning scores were 90.0-95.0 for arm 1 and 82.0-90.9 for arm 2 (Table 4-35 Table 4-42; and Table 4-7). The mean (SD) GHS/QoL scores were well controlled and similar for both treatment arms at baseline (77.8 [14.4] for arm 1 and 80.2 [20.9] for arm 2) (Table 4-24). Over Weeks 2-28, the mean GHS/QoL scores were 69.2-77.3 for arm 1 and 66.7-75.8 for arm 2 (Table 4-24-Table 4-32; and Table 4-7).

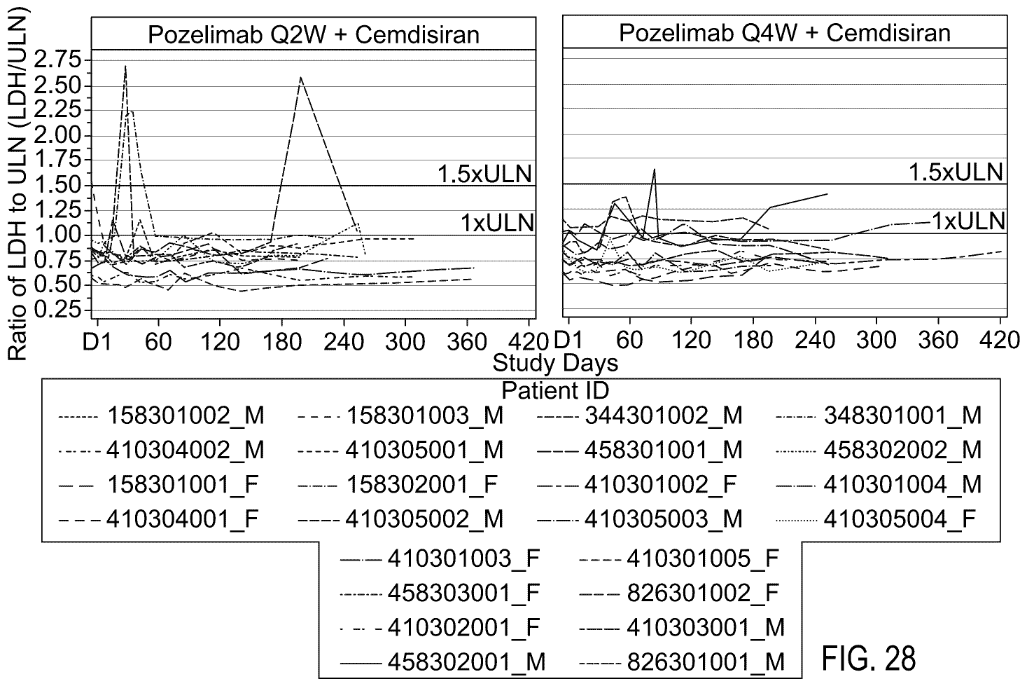

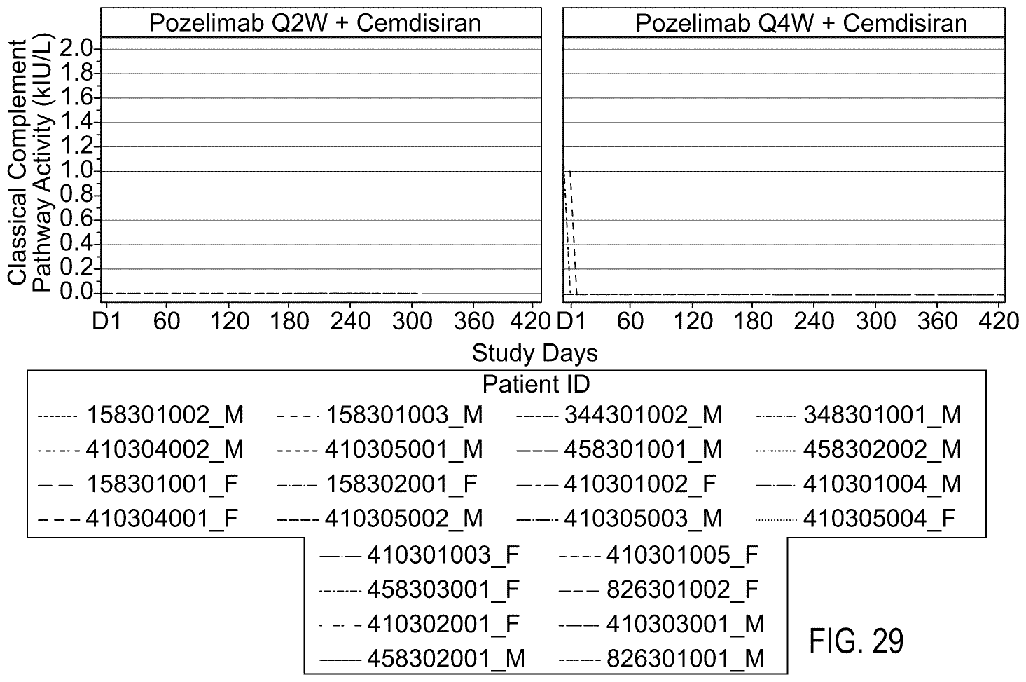

At the time of this analysis, all patients had completed the OLTP; 23 entered the optional OLEP (Table 4-8). During the OLTP, 20 patients (83.3%) maintained control of lactate dehydrogenase (LDH; ≤1.5× upper limit of normal [ULN]) at all timepoints (most patients maintained an LDH<1.0×ULN) (FIG. 28). Most patients (75%, n=18) met the criteria for hemoglobin stabilization (did not receive a blood transfusion and had no decrease in hemoglobin ≥2 g/dL) (Table 4-10). Two patients had breakthrough hemolysis (both associated with complement activating conditions) and required a blood transfusion. CH50, a measure of total complement hemolysis activity, remained fully suppressed in all patients at all post-baseline time-points (Table 4-12A, Table 4-12B and FIG. 29).

At data cut-off, 16 patients (66.7%; seven from arm-1 and nine from arm-2) experienced a total of 46 treatment-emergent adverse events (TEAEs); none leading to treatment discontinuation; 43 were of mild-to-moderate intensity (Table 4-11). Three severe TEAEs occurred in 2 patients, one patient had anemia, another patient had 2 events—gastroenteritis in association with an event of breakthrough hemolysis. Three patients in arm 2 had 1 serious TEAE each (COVID-19, upper respiratory tract infection, aforementioned gastroenteritis). No serious/severe TEAEs were considered related to study treatment, and all resolved. There were no meningococcal infections, thrombotic events, or deaths (Table 4-12 and Table 4-13).

### Summary/Conclusion

Patients with PNH who transitioned from Pozelimab monotherapy had improved baseline scores compared with pre-treatment for their GHS/QoL, physical functioning, and fatigue scores. Improvements in these scores were maintained by the combination treatment through to Week 28, particularly with the Pozelimab Q4W and Cemdisiran dose regimen. Although further validation of these finding in a larger data set would be needed, this evidence suggests that Pozelimab and Cemdisiran combination therapy, particularly the Q4W regimen, improves and maintains improvements in patient fatigue, physical functioning, and QoL.

The combination of Pozelimab and Cemdisiran was generally well tolerated in patients with PNH, regardless of treatment arm. Overall, 83.3% maintained adequate control of hemolysis (Table 4-9), most maintaining normalization of LDH with 75% achieving hemoglobin stabilization during the OLTP. These findings support the ongoing development of this combination therapy in the treatment of patients with PNH.

TABLE 4-7
Patient-reported outcomes

| | | | EORTC-QLQ-C30 physical | | EORTC-QLQ-C30 GHS/QoL | |
|---|---|---|---|---|---|---|
| | FACIT-Fatigue over time^{a} | | functioning over time^{b} | | over time^{b} | |
| | Pozelimab | Pozelimab | Pozelimab | Pozelimab | Pozelimab | Pozelimab |
| | Q4W + | Q2W + | Q4W + | Q2W + | Q4W + | Q2W + |
| | Cemdisiran | Cemdisiran | Cemdisiran | Cemdisiran | Cemdisiran | Cemdisiran |
| Visit | (n = 12) | (n = 12) | (n = 12) | (n = 12) | (n = 12) | (n = 12) |
| Pre- | 22 | | 22 | | 22 | |
| treatment, n | | | | | | |
| Mean | 32.3 (15.2) | | 70.9 (22.5) | | 60.6 (22.4) | |
| [SD]^{c} | | | | | | |
| Baseline, n^{d} | 9 | 8 | 9 | 8 | 9 | 8 |
| Mean | 45.4 (5.6) | 45.6 (3.6) | 93.3 (8.8) | 94.2 (9.0) | 77.8 (14.4) | 80.2 (20.9) |
| (SD) | | | | | | |
| Week 2, n | 10 | 10 | 10 | 10 | 10 | 10 |
| Mean | 45.2 (4.2) | 42.8 (8.4) | 94.0 (8.6) | 85.3 (18.8) | 75.8 (17.8) | 75.0 (16.7) |
| (SD) | | | | | | |
| Week 4, n | 12 | 11 | 12 | 11 | 12 | 11 |
| Mean | 40.3 (11.1) | 39.9 (9.6) | 90.0 (9.2) | 84.2 (19.4) | 70.8 (25.5) | 75.8 (22.5) |
| (SD) | | | | | | |
| Week 8, n | 10 | 12 | 10 | 12 | 10 | 12 |
| Mean | 42.9 (6.6) | 39.3 (11.3) | 92.7 (7.3) | 84.4 (19.4) | 69.2 (20.8) | 70.1 (26.2) |
| (SD) | | | | | | |
| Week 12, n | 12 | 12 | 12 | 12 | 12 | 12 |
| Mean | 43.5 (7.0) | 40.3 (8.9) | 91.1 (9.1) | 87.8 (15.5) | 71.5 (15.3) | 72.9 (19.5) |
| (SD) | | | | | | |
| Week 16, n | 12 | 12 | 12 | 12 | 12 | 12 |
| Mean | 44.0 (5.1) | 40.8 (11.3) | 90.0 (9.6) | 85.0 (19.9) | 72.2 (22.0) | 68.8 (28.9) |
| (SD) | | | | | | |
| Week 20, n | 11 | 10 | 11 | 10 | 11 | 10 |
| Mean | 43.4 (6.8) | 36.5 (12.8) | 93.9 (8.1) | 82.0 (18.9) | 77.3 (20.4) | 66.7 (24.5) |
| (SD | | | | | | |
| Week 24, n | 12 | 11 | 12 | 11 | 12 | 11 |
| Mean | 44.1 (7.3) | 42.9 (9.1) | 90.6 (9.2) | 86.1 (19.0) | 75.7 (19.0) | 73.5 (29.3) |
| (SD) | | | | | | |
| Week 28, n | 12 | 11 | 12 | 11 | 12 | 11 |
| Mean | 43.6 (6.7) | 41.7 (10.2) | 95.0 (5.8) | 90.9 (16.7) | 75.0 (16.7) | 73.5 (23.8) |
| (SD) | | | | | | |

^{a}FACIT-Fatigue: scores range from 0 to 52, with higher scores indicating less fatigue.
^{b}EORTC QLQ-C30: scores range from 0 to 100; a high score for a functional scale represents a high/healthy level of functioning, a high score for the GHS/QoL represents a high QoL.
^{c}Pre-treatment baseline values are given for the previous phase 2 trial where all patients received open-label Pozelimab monotherapy (NCT03946748).
^{d}Seven patients in this study completed PRO instruments after receiving baseline treatment and thus were removed from analysis at this timepoint.
EORTC-QLQ-C30, European Organization for Research and Treatment of Cancer; Quality-of-Life Questionnaire core 30 items; FACIT-Fatigue, Functional Assessment of Chronic Illness Therapy-Fatigue; GHS, global health status; Q2W, every 2 weeks; Q4W, every 4 weeks; QoL, quality of life; SD, standard deviation.

TABLE 4-8
Disposition Table (Safety Analysis Set)-Week 28

| | Pozelimab Q2W + | Pozelimab Q4W + | |
|---|---|---|---|
| | Cemdisiran | Cemdisiran | Total |
| | (N = 12) | (N = 12) | (N = 24) |
| Subjects Randomized | 12 (100%) | 12 (100%) | 24 (100%) |
| Safety Analysis Set | 12 (100%) | 12 (100%) | 24 (100%) |
| Full Analysis Set | 12 (100%) | 12 (100%) | 24 (100%) |
| Subjects Who Received Open-Label | 12 (100%) | 12 (100%) | 24 (100%) |
| Treatment | | | |
| Status | | | |
| COMPLETED | 12 (100%) | 12 (100%) | 24 (100%) |
| Subjects Who Received Open-Label | 11 (91.7%) | 12 (100%) | 23 (95.8%) |
| Extension Treatment | | | |
| Treatment Status | | | |
| ONGOING | 10 (90.9%) | 12 (100%) | 22 (95.7%) |
| DISCONTINUED | 1 (9.1%) | 0 | 1 (4.3%) |
| Discontinuation Reason: | | | |
| SUBJECT DECISION | 1 (100%) | 0 | 1 (100%) |
| Subjects' Study Status | | | |
| ONGOING | 10 (83.3%) | 12 (100%) | 22 (91.7%) |
| DISCONTINUED | 2 (16.7%) | 0 | 2 (8.3%) |
| Discontinuation Reason: | | | |
| SUBJECT DECISION | 2 (100%) | 0 | 2 (100%) |

Ongoing is defined as not having completed all phases and not having discontinued the study. Subjects who have completed the study are those who have finished all phases of the study (OLTP, OLEP and/or safety follow-up) that they entered.

TABLE 4-9
Maintenance of Adequate Control of Hemolysis from Baseline to Week 28

| | Pozelimab | | |
|---|---|---|---|
| | Q2W + | Q4W + | |
| | Cemdisiran | Cemdisiran | Total |
| | (N = 12) | (N = 12) | (N = 24) |
| Proportion of patients with maintaining adequate | 9 (75.0%) | 11 (91.7%) | 20 (83.3%) |
| control from baseline to week 28 | | | |
| Normal Approximation 95% Confidence Interval (%) | 50.5-99.5 | 76.0-100.0 | 68.4-98.2 |
| Clopper Pearson (95%) Confidence Interval (%) | 42.8-94.5 | 61.5-99.8 | 62.6-95.3 |

TABLE 4-10
Hemoglobin stabilization from Baseline through Week 28

| | Pozelimab | | |
|---|---|---|---|
| | Q2W + | Q4W + | |
| | Cemdisiran | Cemdisiran | Total |
| | (N = 12) | (N = 12) | (N = 24) |
| Proportion of patients with hemoglobin stabilization | 8 (66.7%) | 10 (83.3%) | 18 (75.0%) |
| from baseline to week 28 | | | |
| Normal Approximation 95% Confidence Interval (%) | 40.0-93.3 | 62.2-100.0 | 57.7-92.3 |
| Clopper Pearson (95%) Confidence Interval (%) | 34.9-90.1 | 51.6-97.9 | 53.3-90.2 |

TABLE 4-11
Treatment Emergent Adverse Events (TEAE)

| | Pozelimab Q2W + | Pozelimab Q4W + | | |
|---|---|---|---|---|
| | Cemdisiran | Cemdisiran | Total | |
| | (N = 12) | (N = 12) | (N = 24) | |
| Total Number of TEAEs | 32 | 14 | 46 | |
| Total Number of Serious TEAEs | 3 | 0 | 3 | |
| Total Number of Related TEAEs | 8 | 7 | 15 | |
| Total Number of Severe TEAEs | 3 | 0 | 3 | |
| Subjects with any TEAE | 9 (75.0%) | 7 (58.3%) | 16 | (66.7%) |
| Subjects with any Serious TEAE | 3 (25.0%) | 0 | 3 | (12.5%) |
| Subjects with any Related TEAE | 3 (25.0%) | 4 (33.3%) | 7 | (29.2%) |
| Subjects with any Severe TEAE | 2 (16.7%) | 0 | 2 | (8.3%) |
| Subjects with any TEAE Leading to | 0 | 0 | 0 | |
| Discontinuation of Any Study Drug | | | | |

| | | | |
|---|---|---|---|
| Subjects with any TEAE Leading to Death | 0 | 0 | 0 |

TABLE 4-12A
Severe TEAEs
Treatment Group: Pozelimab Q2W + Cemdisiran

| | | | AE Start Date | | Related | | | Is AE |
|---|---|---|---|---|---|---|---|---|
| | AE | System Organ Class/ | Time (Start | TEAE?/ | to Poz | Action Taken | | of |
| | Num- | Preferred term/ | Day)/Stop Date | SAE?/ | IV/Poz | with Poz IV/ | | Special |
| Subject ID | ber | Investigator Term | Time (Stop Day) | Severity | SC/Cemd | Poz SC/Cemd | Outcome | Interest? |
| 348301001 | 002 | Blood and lymphatic system | (25)/ | Y/ | N/ | Not Applicable/ | RECOVERED/ | N |
| | | disorders/Breakthrough | (57) | N/ | N/ | Dose Not Changed/ | RESOLVED | |
| | | haemolysis/ | | MOD- | N | Dose Not Changed | WITH | |
| | | BREAKTHROUGH HEMOLYSIS | | ERATE | | | SEQUELAE | |
| | 003 | Infections and infestations/ | (35)/ | Y/ | N/ | Not Applicable/ | RECOVERED/ | N |
| | | Chlamydial infection/ | (50) | N/ | N/ | Dose Not Changed/ | RESOLVED | |
| | | CHLAMYDIA TRACHOMATIS | | MOD- | N | Dose Not Changed | | |
| | | INFECTION | | ERATE | | | | |
| | 004 | Blood and lymphatic system | (113)/ | Y/ | N/ | Not Applicable/ | RECOVERED/ | N |
| | | disorders/Haemolysis/ | (114) | N/ | N/ | Dose Not Changed/ | RESOLVED | |
| | | UNPROVED SUSPECTED | | MOD- | N | Dose Not Changed | | |
| | | HEMOLISIS | | ERATE | | | | |
| | 005 | Blood and lymphatic system | (57)/ | Y/ | N/ | Not Applicable/ | RECOVERED/ | N |
| | | disorders/Anaemia/ | (112) | N/ | N/ | Dose Not Changed/ | RESOLVED | |
| | | ANEMIA | | MOD- | N | Dose Not Changed | | |
| | | | | ERATE | | | | |
| | 006 | Blood and lymphatic system | (113)/ | Y/ | N/ | Not Applicable/ | RECOVERED/ | N |
| | | disorders/Anaemia/ | (127) | N/ | N/ | Dose Not Changed/ | RESOLVED | |
| | | WORSENING ANEMIA | | SEVERE | N | Dose Not Changed | | |

TABLE 4-12B
Severe TEAEs-continued
Treatment Group: Pozelimab Q2W + Cemdisiran

| | | | AE Start Date | | Related | | | Is AE |
|---|---|---|---|---|---|---|---|---|
| | AE | System Organ Class/ | Time (Start | TEAE?/ | to Poz | Action Taken | | of |
| | Num- | Preferred term/ | Day)/Stop Date | SAE?/ | IV/Poz | with Poz IV/ | | Special |
| Subject ID | ber | Investigator Term | Time (Stop Day) | Severity | SC/Cemd | Poz SC/Cemd | Outcome | Interest? |
| 410304002 | 003 | General disorders and | (255)/ | Y/ | N/ | Not Applicable/ | RECOVERED/ | Y |
| | | administration site conditions/ | (323) | N/ | N/ | Dose Not Changed/ | RESOLVED | |
| | | Injection site reaction/ | | MILD | Y | Dose Not Changed | | |
| | | INJECTION SITE REACTION | | | | | | |
| 458301001 | 001 | Musculoskeletal and connective | (1)/ | Y/ | N/ | Not Applicable/ | RECOVERED/ | N |
| | | tissue disorders/Muscle spasms/ | (1) | N/ | N/ | Dose Not Changed/ | RESOLVED | |
| | | MUSCLE SPASM | | MILD | Y | Dose Not Changed | | |
| | 002 | Investigations/ | (57)/ | Y/ | N/ | Not Applicable/ | RECOVERED/ | N |
| | | Transaminases increased/ | ONGOING | N/ | N/ | Dose Not Changed/ | RESOLVING | |
| | | ELEVATED AST AND ALT | | MILD | N | Dose Not Changed | | |
| | 003 | Blood and lymphatic system | (198)/ | Y/ | N/ | Not Applicable/ | RECOVERED/ | N |
| | | disorders/Breakthrough | (201) | N/ | N/ | Dose Not Changed/ | RESOLVED | |
| | | haemolysis/ | | SEVERE | N | Dose Not Changed | | |
| | | BREAKTHROUGH HEMOLYSIS | | | | | | |
| | 005 | Infections and infestations/ | (194)/ | Y/ | N/ | Not Applicable/ | RECOVERED/ | N |
| | | Gastroenteritis/ | (201) | N/ | N/ | Dose Not Changed/ | RESOLVED | |
| | | INFECTION; ACUTE | | SEVERE | N | Dose Not Changed | | |
| | | GASTROENTERITIS | | | | | | |

TABLE 4-13
Baseline Pre-treatment FACIT-Fatigue
from 1852 (Full Analysis Set)

| | Pozelimab |
|---|---|
| Visit | (N = 24) |
| Baseline | |
| Value | |
| n | 22 |
| Mean (SD) | 32.3 (15.15) |
| Median | 36.5 |
| Q1:Q3 | 18.0:45.0 |
| Min:Max | 5:52 |

TABLE 4-14
FACIT-Fatigue over Time in 2092-Baseline to Week 28 (Full Analysis Set) (pt. 1)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Baseline | | | | | | |
| Observed Value | | | | | | |
| n | 8 | | 9 | | 17 | |
| Mean (SD) | 45.6 | (3.62) | 45.4 | (5.55) | 45.5 | (4.60) |
| Median | 46.0 | | 48.0 | | 48.0 | |
| Q1:Q3 | 43:49 | | 43:49 | | 43:49 | |
| Min:Max | 40:50 | | 35:50 | | 35:50 | |
| Day 15 | | | | | | |
| Observed Value | | | | | | |
| n | 10 | | 10 | | 20 | |
| Mean (SD) | 42.8 | (8.40) | 45.2 | (4.24) | 44.0 | (6.59) |
| Median | 45.5 | | 46.5 | | 46.0 | |
| Q1:Q3 | 41:49 | | 40:48 | | 41:49 | |
| Min:Max | 24:51 | | 39:50 | | 24:51 | |
| Change from baseline | | | | | | |
| n | 8 | | 9 | | 17 | |
| Mean (SD) | −4.0 | (7.96) | −0.3 | (2.45) | −2.1 | (5.86) |

| Median | −1.0 | −1.0 | −1.0 |
|---|---|---|---|
| Q1:Q3 | −9:2 | −1:1 | −3:2 |
| Min:Max | −20:3 | −4:4 | −20:4 |

TABLE 4-15
FACIT-Fatigue over Time in 2092-Baseline to Week 28 (Full Analysis Set) (pt. 2)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Day 15 | | | | | | |
| Percent change from baseline | | | | | | |
| n | 8 | | 9 | | 17 | |
| Mean (SD) | −8.9 | (18.11) | −0.3 | (6.09) | −4.4 | (13.47) |
| Median | −2.1 | | −2.0 | | −2.0 | |
| Q1:Q3 | −19:4 | | −2:2 | | −6:4 | |
| Min:Max | −45:8 | | −9:11 | | −45:11 | |
| Day 29 | | | | | | |
| Observed Value | | | | | | |
| n | 11 | | 12 | | 23 | |
| Mean (SD) | 39.9 | (9.63) | 40.3 | (11.08) | 40.1 | (10.18) |
| Median | 42.0 | | 44.0 | | 44.0 | |
| Q1:Q3 | 30:50 | | 39:48 | | 33:49 | |
| Min:Max | 25:52 | | 10:50 | | 10:52 | |
| Change from baseline | | | | | | |
| n | 8 | | 9 | | 17 | |
| Mean (SD) | −5.9 | (6.88) | −6.0 | (8.80) | −5.9 | (7.71) |

| Median | −5.5 | −3.0 | −4.0 |
|---|---|---|---|
| Q1:Q3 | −11:0 | −11:1 | −11:1 |
| Min:Max | −17:2 | −25:1 | −25:2 |

TABLE 4-16
FACIT-Fatigue over Time in 2092-Baseline to Week 28 (Full Analysis Set) (pt. 3)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Day 29 | | | | | | |
| Percent change from baseline | | | | | | |
| n | 8 | | 9 | | 17 | |
| Mean (SD) | −13.6 | (15.99) | −14.9 | (24.13) | −14.3 | (20.08) |
| Median | −11.4 | | −6.4 | | −8.0 | |
| Q1:Q3 | −25:0 | | −22:2 | | −22:2 | |
| Min:Max | −40:4 | | −71:3 | | −71:4 | |
| Day 57 | | | | | | |
| Observed Value | | | | | | |
| n | 12 | | 10 | | 22 | |
| Mean (SD) | 39.3 | (11.25) | 42.9 | (6.61) | 40.9 | (9.41) |
| Median | 42.5 | | 45.0 | | 44.0 | |
| Q1:Q3 | 30:50 | | 39:47 | | 37:49 | |
| Min:Max | 18:52 | | 29:51 | | 18:52 | |
| Change from baseline | | | | | | |
| n | 8 | | 8 | | 16 | |
| Mean (SD) | −8.0 | (9.93) | −2.9 | (3.98) | −5.4 | (7.77) |

| Median | −5.5 | −2.5 | −3.0 |
|---|---|---|---|
| Q1:Q3 | −17:1 | −6:1 | −9:1 |
| Min:Max | −24:2 | −10:1 | −24:2 |

TABLE 4-17
FACIT-Fatigue over Time in 2092-Baseline to Week 28 (Full Analysis Set) (pt. 4)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Day 57 | | | | | | |
| Percent change from baseline | | | | | | |
| n | 8 | | 8 | | 16 | |
| Mean (SD) | −18.1 | (22.70) | −6.6 | (9.00) | −12.3 | (17.71) |
| Median | −11.9 | | −5.1 | | −6.8 | |
| Q1:Q3 | −36:2 | | −14:2 | | −19:2 | |
| Min:Max | −57:4 | | −20:3 | | −57:4 | |
| Day 85 | | | | | | |
| Observed Value | | | | | | |
| n | 12 | | 12 | | 24 | |
| Mean (SD) | 40.3 | (8.92) | 43.5 | (7.01) | 41.9 | (8.02) |
| Median | 41.5 | | 48.0 | | 42.5 | |
| Q1:Q3 | 36:46 | | 36:48 | | 36:48 | |
| Min:Max | 22:52 | | 32:52 | | 22:52 | |
| Change from baseline | | | | | | |
| n | 8 | | 9 | | 17 | |
| Mean (SD) | −4.8 | (6.50) | −2.0 | (2.92) | −3.3 | (4.97) |

| Median | −5.0 | −2.0 | −2.0 |
|---|---|---|---|
| Q1:Q3 | −8:0 | −3:0 | −6:0 |
| Min:Max | −16:4 | −6:3 | −16:4 |

TABLE 4-18
FACIT-Fatigue over Time in 2092-Baseline to Week 28 (Full Analysis Set) (pt. 5)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Day 85 | | | | | | |
| Percent change from baseline | | | | | | |
| n | 8 | | 9 | | 17 | |
| Mean (SD) | −10.7 | (14.36) | −4.8 | (6.56) | −7.6 | (11.00) |
| Median | −10.7 | | −4.0 | | −5.0 | |
| Q1:Q3 | −18:0 | | −9:0 | | −14:0 | |
| Min:Max | −36:8 | | −14:6 | | −36:8 | |
| Day 113 | | | | | | |
| Observed Value | | | | | | |
| n | 12 | | 12 | | 24 | |
| Mean (SD) | 40.8 | (11.33) | 44.0 | (5.12) | 42.4 | (8.76) |
| Median | 43.5 | | 44.0 | | 44.0 | |
| Q1:Q3 | 37:48 | | 41:48 | | 41:48 | |
| Min:Max | 14:52 | | 33:51 | | 14:52 | |
| Change from baseline | | | | | | |
| n | 8 | | 9 | | 17 | |
| Mean (SD) | −5.6 | (10.76) | −1.7 | (3.24) | −3.5 | (7.75) |

| Median | −3.5 | −2.0 | −3.0 |
|---|---|---|---|
| Q1:Q3 | −8:2 | −4:1 | −5:1 |
| Min:Max | −30:4 | −6:3 | −30:4 |

TABLE 4-19
FACIT-Fatigue over Time in 2092-Baseline to Week 28 (Full Analysis Set) (pt. 6)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Day 113 | | | | | | |
| Percent change from baseline | | | | | | |
| n | 8 | | 9 | | 17 | |
| Mean (SD) | −12.8 | (24.38) | −3.5 | (7.10) | −7.9 | (17.55) |
| Median | −7.5 | | −5.7 | | −6.8 | |
| Q1:Q3 | −17:3 | | −9:2 | | −10:2 | |
| Min:Max | −68:8 | | −12:8 | | −68:8 | |
| Day 141 | | | | | | |
| Observed Value | 10 | | 11 | | 21 | |
| n | | | | | | |
| Mean (SD) | 36.5 | (12.76) | 43.4 | (6.77) | 40.1 | (10.42) |
| Median | 38.0 | | 46.0 | | 44.0 | |
| Q1:Q3 | 25:47 | | 42:48 | | 34:47 | |
| Min:Max | 19:52 | | 26:50 | | 19:52 | |
| Change from baseline | | | | | | |
| n | 8 | | 9 | | 17 | |
| Mean (SD) | −8.1 | (10.52) | −3.0 | (4.03) | −5.4 | (7.97) |

| Median | −5.0 | −4.0 | −4.0 |
|---|---|---|---|
| Q1:Q3 | −17:0 | −6:0 | −8:0 |
| Min:Max | −25:4 | −9:4 | −25:4 |

TABLE 4-20
FACIT-Fatigue over Time in 2092-Baseline to Week 28 (Full Analysis Set) (pt. 7)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Day 141 | | | | | | |
| Percent change from baseline | | | | | | |
| n | 8 | | 9 | | 17 | |
| Mean (SD) | −18.7 | (23.61) | −6.9 | (10.40) | −12.4 | (18.30) |
| Median | −11.8 | | −8.0 | | −8.0 | |
| Q1:Q3 | −39:0 | | −12:0 | | −19:0 | |
| Min:Max | −57:8 | | −26:11 | | −57:11 | |
| Day 169 | | | | | | |
| Observed Value | | | | | | |
| n | 11 | | 12 | | 23 | |
| Mean (SD) | 42.9 | (9.09) | 44.1 | (7.30) | 43.5 | (8.04) |
| Median | 45.0 | | 47.0 | | 46.0 | |
| Q1:Q3 | 40:52 | | 40:49 | | 40:50 | |
| Min:Max | 21:52 | | 26:51 | | 21:52 | |
| Change from baseline | | | | | | |
| n | 8 | | 9 | | 17 | |
| Mean (SD) | −4.6 | (8.63) | −1.8 | (3.56) | −3.1 | (6.41) |

| Median | −2.5 | −1.0 | −2.0 |
|---|---|---|---|
| Q1:Q3 | −8:2 | −3:1 | −5:1 |
| Min:Max | −23:4 | −9:2 | −23:4 |

TABLE 4-21
FACIT-Fatigue over Time in 2092-Baseline to Week 28 (Full Analysis Set) (pt. 8)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| FACIT Fatigue | | | | | | |
| Day 169 | | | | | | |
| Percent change from baseline | | | | | | |
| n | 8 | | 9 | | 17 | |
| Mean (SD) | −10.4 | (19.39) | −4.7 | (9.38) | −7.4 | (14.74) |
| Median | −5.4 | | −2.6 | | −4.1 | |
| Q1:Q3 | −18:3 | | −6:2 | | −12:3 | |
| Min:Max | −52:8 | | −26:4 | | −52:8 | |
| Day 197 | | | | | | |
| Observed Value | | | | | | |
| n | 11 | | 12 | | 23 | |
| Mean (SD) | 41.7 | (10.15) | 43.6 | (6.68) | 42.7 | (8.37) |
| Median | 43.0 | | 46.0 | | 45.0 | |
| Q1:Q3 | 40:46 | | 40:48 | | 40:48 | |
| Min:Max | 15:52 | | 28:51 | | 15:52 | |
| Change from baseline | | | | | | |
| n | 8 | | 9 | | 17 | |
| Mean (SD) | −5.5 | (9.90) | −2.0 | (4.36) | −3.6 | (7.46) |

| Median | −2.0 | −2.0 | −2.0 |
|---|---|---|---|
| Q1:Q3 | −6:−1 | −5:2 | −5:0 |
| Min:Max | −29:2 | −9:4 | −29:4 |

TABLE 4-22
FACIT-Fatigue over Time in 2092-Baseline to Week 28 (Full Analysis Set) (pt. 9)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| FACIT Fatigue | | | |
| Day 197 | | | |
| Percent change from baseline | | | |
| n | 8 | 9 | 17 |
| Mean (SD) | −12.4 (22.45) | −4.7 (10.08) | −8.3 (16.95) |
| Median | −4.2 | −4.0 | −4.0 |
| Q1:Q3 | −13:−1 | −11:4 | −13:0 |
| Min:Max | −66:4 | −20:9 | −66:9 |

TABLE 4-23
Baseline Pre-treatment EORTC-QLQ-C30
GHS/QoL from 1852 (Full Analysis Set)

| | Pozelimab |
|---|---|
| Visit | (N = 24) |
| Baseline | |
| Value | |
| n | 22 |
| Mean (SD) | 60.606 (22.4466) |
| Median | 62.500 |
| Q1:Q3 | 50.000:75.000 |
| Min:Max | 16.67:100.00 |

TABLE 4-24
EORTC-QLQ-C30 GHS/QoL over Time in 2092-Baseline
to Week 28 (Full Analysis Set) (pt. 1)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Baseline | | | |
| Observed Value | | | |
| n | 8 | 9 | 17 |
| Mean (SD) | 80.2 (20.86) | 77.8 (14.43) | 78.9 (17.21) |
| Median | 83.3 | 83.3 | 83.3 |
| Q1:Q3 | 67:100 | 67:83 | 67:83 |
| Min:Max | 42:100 | 50:100 | 42:100 |
| Day 15 | | | |
| Observed Value | | | |
| n | 10 | 10 | 20 |
| Mean (SD) | 75.0 (16.67) | 75.8 (17.76) | 75.4 (16.77) |
| Median | 79.2 | 79.2 | 79.2 |
| Q1:Q3 | 67:83 | 67:83 | 67:83 |
| Min:Max | 50:100 | 50:100 | 50:100 |
| Change from baseline | | | |
| n | 8 | 9 | 17 |
| Mean (SD) | −5.2 (8.84) | −0.9 (15.84) | −2.9 (12.82) |
| Median | −4.2 | 0.0 | 0.0 |
| Q1:Q3 | −13:0 | 0:8 | −8:0 |
| Min:Max | −17:8 | −33:17 | −33:17 |

TABLE 4-25
EORTC-QLQ-C30 GHS/QoL over Time in 2092-Baseline
to Week 28 (Full Analysis Set) (pt. 2)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 15 | | | |
| Percent change from baseline | | | |
| n | 8 | 9 | 17 |
| Mean (SD) | −5.0 (13.48) | −0.3 (19.94) | −2.5 (16.86) |
| Median | −4.2 | 0.0 | 0.0 |
| Q1:Q3 | −13:0 | 0:13 | −10:0 |
| Min:Max | −25:20 | −40:25 | −40:25 |
| Day 29 | | | |
| Observed Value | | | |
| n | 11 | 12 | 23 |
| Mean (SD) | 75.8 (22.50) | 70.8 (25.50) | 73.2 (23.70) |
| Median | 83.3 | 70.8 | 75.0 |
| Q1:Q3 | 67:100 | 58:92 | 67:100 |
| Min:Max | 33:100 | 17:100 | 17:100 |
| Change from baseline | | | |
| n | 8 | 9 | 17 |
| Mean (SD) | −5.2 (12.55) | −11.1 (21.65) | −8.3 (17.68) |
| Median | −4.2 | 0.0 | 0.0 |
| Q1:Q3 | −13:0 | −17:0 | −17:0 |
| Min:Max | −25:17 | −50:17 | −50:17 |

TABLE 4-26
EORTC-QLQ-C30 GHS/QoL over Time in 2092-Baseline
to Week 28 (Full Analysis Set) (pt. 3)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 29 | | | |
| Percent change from baseline | | | |
| n | 8 | 9 | 17 |
| Mean (SD) | −7.4 (18.41) | −15.0 (29.58) | −11.4 (24.52) |
| Median | −5.0 | 0.0 | 0.0 |
| Q1:Q3 | −18:0 | −20:0 | −20:0 |
| Min:Max | −38:25 | −75:20 | −75:25 |
| Day 57 | | | |
| Observed Value | | | |
| n | 12 | 10 | 22 |
| Mean (SD) | 70.1 (26.22) | 69.2 (20.81) | 69.7 (23.37) |
| Median | 70.8 | 66.7 | 66.7 |
| Q1:Q3 | 58:92 | 50:92 | 58:92 |
| Min:Max | 17:100 | 42:100 | 17:100 |
| Change from baseline | | | |
| n | 8 | 8 | 16 |
| Mean (SD) | −12.5 (12.60) | −10.4 (16.52) | −11.5 (14.23) |
| Median | −12.5 | −4.2 | −8.3 |
| Q1:Q3 | −21:0 | −21:0 | −21:0 |
| Min:Max | −33:0 | −42:8 | −42:8 |

TABLE 4-27
EORTC-QLQ-C30 GHS/QoL over Time in 2092-Baseline
to Week 28 (Full Analysis Set) (pt. 4)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 57 | | | |
| Percent change from baseline | | | |
| n | 8 | 8 | 16 |
| Mean (SD) | −19.9 (23.20) | −13.4 (20.91) | −16.7 (21.60) |
| Median | −14.6 | −5.0 | −11.3 |
| Q1:Q3 | −35:0 | −29:0 | −29:0 |
| Min:Max | −60:0 | −50:10 | −60:10 |
| Day 85 | | | |
| Observed Value | | | |
| n | 12 | 12 | 24 |
| Mean (SD) | 72.9 (19.50) | 71.5 (15.27) | 72.2 (17.14) |
| Median | 70.8 | 66.7 | 66.7 |
| Q1:Q3 | 67:88 | 67:83 | 67:83 |
| Min:Max | 33:100 | 50:100 | 33:100 |
| Change from baseline | | | |
| n | 8 | 9 | 17 |
| Mean (SD) | −6.3 (13.18) | −6.5 (13.03) | −6.4 (12.68) |
| Median | 0.0 | 0.0 | 0.0 |
| Q1:Q3 | −13:0 | −17:0 | −17:0 |
| Min:Max | −33:8 | −33:8 | −33:8 |

TABLE 4-28
EORTC-QLQ-C30 GHS/QoL over Time in 2092-Baseline
to Week 28 (Full Analysis Set) (pt. 5)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 85 | | | |
| Percent change from baseline | | | |
| n | 8 | 9 | 17 |
| Mean (SD) | −7.3 (20.51) | −7.8 (15.63) | −7.5 (17.50) |
| Median | 0.0 | 0.0 | 0.0 |
| Q1:Q3 | −14:0 | −20:0 | −20:0 |
| Min:Max | −50:20 | −40:10 | −50:20 |
| Day 113 | | | |
| Observed Value | | | |
| n | 12 | 12 | 24 |
| Mean (SD) | 68.8 (28.89) | 72.2 (22.00) | 70.5 (25.18) |
| Median | 70.8 | 79.2 | 75.0 |
| Q1:Q3 | 58:92 | 58:83 | 58:83 |
| Min:Max | 0:100 | 25:100 | 0:100 |
| Change from baseline | | | |
| n | 8 | 9 | 17 |
| Mean (SD) | −11.5 (23.96) | −10.0 (19.44) | −10.8 (20.99) |
| Median | 0.0 | 0.0 | 0.0 |
| Q1:Q3 | −13:0 | −17:0 | −17:0 |
| Min:Max | −67:0 | −58:0 | −67:0 |

TABLE 4-29
EORTC-QLQ-C30 GHS/QoL over Time in 2092-Baseline
to Week 28 (Full Analysis Set) (pt. 6)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 113 | | | |
| Percent change from baseline | | | |
| n | 8 | 9 | 17 |
| Mean (SD) | −16.3 (35.43) | −12.8 (23.60) | −14.4 (28.83) |
| Median | 0.0 | 0.0 | 0.0 |
| Q1:Q3 | −15:0 | −20:0 | −20:0 |
| Min:Max | −100:0 | −70:0 | −100:0 |
| Day 141 | | | |
| Observed Value | | | |
| n | 10 | 11 | 21 |
| Mean (SD) | 66.7 (24.53) | 77.3 (20.44) | 72.2 (22.57) |
| Median | 62.5 | 83.3 | 75.0 |
| Q1:Q3 | 50:83 | 50:100 | 50:92 |
| Min:Max | 33:100 | 50:100 | 33:100 |
| Change from baseline | | | |
| n | 8 | 9 | 17 |
| Mean (SD) | −9.4 (14.39) | −3.7 (17.73) | −6.4 (16.01) |
| Median | −4.2 | 0.0 | 0.0 |
| Q1:Q3 | −21:0 | −17:0 | −17:0 |
| Min:Max | −33:8 | −33:25 | −33:25 |

TABLE 4-30
EORTC-QLQ-C30 GHS/QoL over Time in 2092-Baseline
to Week 28 (Full Analysis Set) (pt. 7)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 141 | | | |
| Percent change from baseline | | | |
| n | 8 | 9 | 17 |
| Mean (SD) | −11.8 (22.60) | −4.2 (23.39) | −7.7 (22.63) |
| Median | −5.0 | 0.0 | 0.0 |
| Q1:Q3 | −27:0 | −20:0 | −20:0 |
| Min:Max | −50:00 | −40:38 | −50:38 |
| Day 169 | | | |
| Observed Value | | | |
| n | 11 | 12 | 23 |
| Mean (SD) | 73.5 (29.30) | 75.7 (18.96) | 74.6 (23.90) |
| Median | 75.0 | 75.0 | 75.0 |
| Q1:Q3 | 67:100 | 67:92 | 67:100 |
| Min:Max | 0:100 | 42:100 | 0:100 |
| Change from baseline | | | |
| n | 8 | 9 | 17 |
| Mean (SD) | −9.4 (24.98) | −4.6 (16.72) | −6.9 (20.46) |
| Median | 0.0 | 0.0 | 0.0 |
| Q1:Q3 | −13:0 | −8:0 | −8:0 |
| Min:Max | −67:17 | −42:17 | −67:17 |

TABLE 4-31
EORTC-QLQ-C30 GHS/QoL over Time in 2092-Baseline
to Week 28 (Full Analysis Set) (pt. 8)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 169 | | | |
| Percent change from baseline | | | |
| n | 8 | 9 | 17 |
| Mean (SD) | −11.3 (39.80) | −5.3 (20.33) | −8.1 (30.15) |
| Median | 0.0 | 0.0 | 0.0 |
| Q1:Q3 | −15:0 | −10:0 | −10:0 |
| Min:Max | −100:40 | −50:20 | −100:40 |
| Day 197 | | | |
| Observed Value | | | |
| n | 11 | 12 | 23 |
| Mean (SD) | 73.5 (23.81) | 75.0 (16.67) | 74.3 (19.93) |
| Median | 66.7 | 75.0 | 66.7 |
| Q1:Q3 | 67:100 | 67:83 | 67:83 |
| Min:Max | 17:100 | 50:100 | 17:100 |
| Change from baseline | | | |
| n | 8 | 9 | 17 |
| Mean (SD) | −9.4 (23.33) | −1.9 (13.03) | −5.4 (18.39) |
| Median | 0.0 | 0.0 | 0.0 |
| Q1:Q3 | −25:0 | −17:0 | −17:0 |
| Min:Max | −50:25 | −17:17 | −50:25 |

TABLE 4-32
EORTC-QLQ-C30 GHS/QoL over Time in 2092-Baseline
to Week 28 (Full Analysis Set) (pt. 9)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Global health status/QoL | | | |
| Day 197 | | | |
| Percent change from baseline | | | |
| n | 8 | 9 | 17 |
| Mean (SD) | −8.5 (38.08) | −2.2 (17.34) | −5.2 (28.20) |
| Median | 0.0 | 0.0 | 0.0 |
| Q1:Q3 | −27:0 | −20:0 | −20:0 |
| Min:Max | −75:60 | −25:25 | −75:60 |

TABLE 4-33
Baseline Pre-treatment EORTIC-QLQ-C30 Physical
Functioning from 1852 (Full Analysis Set)

| | Pozelimab |
|---|---|
| Visit | (N = 24) |
| Baseline | |
| Value | |
| n | 22 |
| Mean (SD) | 70.909 (22.4947) |
| Median | 76.667 |
| Q1:Q3 | 53.333:86.667 |
| Min:Max | 26.67:100.00 |

TABLE 4-34
EORTC-QLQ-C30 Physical Functioning over Time in 2092-
Baseline to Week 28 (Full Analysis Set) (pt. 1)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Physical Functioning | | | |
| Baseline | | | |
| Observed Value | | | |
| n | 8 | 9 | 17 |
| Mean (SD) | 94.2 (9.04) | 93.3 (8.82) | 93.7 (8.65) |
| Median | 96.7 | 100.0 | 100.0 |
| Q1:Q3 | 93:100 | 87:100 | 93:100 |
| Min:Max | 73:100 | 80:100 | 73:100 |
| Day 15 | | | |
| Observed Value | | | |
| n | 10 | 10 | 20 |
| Mean (SD) | 85.3 (18.80) | 94.0 (8.58) | 89.7 (14.90) |
| Median | 93.3 | 100.0 | 93.3 |
| Q1:Q3 | 73:100 | 87:100 | 83:100 |
| Min:Max | 40:100 | 80:100 | 40:100 |
| Change from baseline | | | |
| n | 8 | 9 | 17 |
| Mean (SD) | −10.8 (21.06) | 0.0 (3.33) | −5.1 (15.19) |
| Median | 0.0 | 0.0 | 0.0 |
| Q1:Q3 | −13:0 | 0:0 | 0:0 |
| Min:Max | −60:0 | −7:7 | −60:7 |

TABLE 4-35
EORTC-QLQ-C30 Physical Functioning over Time in 2092-
Baseline to Week 28 (Full Analysis Set) (pt. 2)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Physical Functioning | | | |
| Day 15 | | | |
| Percent change from baseline | | | |
| n | 8 | 9 | 17 |
| Mean (SD) | −11.0 (21.16) | 0.1 (3.45) | −5.2 (15.30) |
| Median | 0.0 | 0.0 | 0.0 |
| Q1:Q3 | −14:0 | 0:0 | 0:0 |
| Min:Max | −60:0 | −7:7 | −60:7 |
| Day 29 | | | |
| Observed Value | | | |
| n | 11 | 12 | 23 |
| Mean (SD) | 84.2 (19.38) | 90.0 (9.21) | 87.2 (14.90) |
| Median | 93.3 | 93.3 | 93.3 |
| Q1:Q3 | 67:100 | 80:97 | 80:100 |
| Min:Max | 47:100 | 73:100 | 47:100 |
| Change from baseline | | | |
| n | 8 | 9 | 17 |
| Mean (SD) | −13.3 (20.78) | −3.0 (9.49) | −7.8 (16.20) |
| Median | −3.3 | 0.0 | 0.0 |
| Q1:Q3 | −27:0 | 0:0 | −7:0 |
| Min:Max | −47:0 | −27:7 | −47:7 |

TABLE 4-36
EORTC-QLQ-C30 Physical Functioning over Time in 2092-
Baseline to Week 28 (Full Analysis Set) (pt. 3)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Physical Functioning | | | |
| Day 29 | | | |
| Percent change from baseline | | | |
| n | 8 | 9 | 17 |
| Mean (SD) | −14.1 (21.46) | −2.8 (9.63) | −8.1 (16.77) |
| Median | −3.3 | 0.0 | 0.0 |
| Q1:Q3 | −28:0 | 0:0 | −7:0 |
| Min:Max | −50:0 | −27:8 | −50:8 |
| Day 57 | | | |
| Observed Value | | | |
| n | 12 | 10 | 22 |
| Mean (SD) | 84.4 (19.35) | 92.7 (7.34) | 88.2 (15.39) |
| Median | 93.3 | 93.3 | 93.3 |
| Q1:Q3 | 80:97 | 87:100 | 87:100 |
| Min:Max | 40:100 | 80:100 | 40:100 |
| Change from baseline | | | |
| n | 8 | 8 | 16 |
| Mean (SD) | −13.3 (23.367) | −0.8 (6.61) | −7.1 (17.80) |
| Median | 0.0 | 0.0 | 0.0 |
| Q1:Q3 | −23:0 | −3:3 | −7:0 |
| Min:Max | −60:0 | −13:7 | −60:7 |

TABLE 4-37
EORTC-QLQ-C30 Physical Functioning over Time in 2092-
Baseline to Week 28 (Full Analysis Set) (pt. 4)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Physical Functioning | | | |
| Day 57 | | | |
| Percent change from baseline | | | |
| n | 8 | 8 | 16 |
| Mean (SD) | −13.7 (23.85) | −0.6 (6.97) | −7.1 (18.28) |
| Median | 0.0 | 0.0 | 0.0 |
| Q1:Q3 | −25:0 | −3:4 | −7:0 |
| Min:Max | −60:0 | −13:8 | −60:8 |
| Day 85 | | | |
| Observed Value | | | |
| n | 12 | 12 | 24 |
| Mean (SD) | 87.8 (15.53) | 91.1 (9.14) | 89.4 (12.58) |
| Median | 93.3 | 93.3 | 93.3 |
| Q1:Q3 | 80:100 | 80:100 | 80:100 |
| Min:Max | 53:100 | 80:100 | 53:100 |
| Change from baseline | | | |
| n | 8 | 9 | 17 |
| Mean (SD) | −10.8 (17.07) | −3.0 (5.88) | −6.7 (12.69) |
| Median | −3.3 | 0.0 | 0.0 |
| Q1:Q3 | −17:0 | −7:0 | −7:0 |
| Min:Max | −47:0 | −13:7 | −47:7 |

TABLE 4-38
EORTC-QLQ-C30 Physical Functioning over Time in 2092-
Baseline to Week 28 (Full Analysis Set) (pt. 5)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Physical Functioning | | | |
| Day 85 | | | |
| Percent change from baseline | | | |
| n | 8 | 9 | 17 |
| Mean (SD) | −11.1 (17.31) | −3.0 (6.07) | −6.8 (12.92) |
| Median | −3.3 | 0.0 | 0.0 |
| Q1:Q3 | −18:0 | −7:0 | −7:0 |
| Min:Max | −47:0 | −13:7 | −47:7 |
| Day 113 | | | |
| Observed Value | | | |
| n | 12 | 12 | 24 |
| Mean (SD) | 85.0 (19.92) | 90.0 (9.64) | 87.5 (15.52) |
| Median | 93.3 | 93.3 | 93.3 |
| Q1:Q3 | 77:100 | 83:97 | 80:100 |
| Min:Max | 40:100 | 73:100 | 40:100 |
| Change from baseline | | | |
| n | 8 | 9 | 17 |
| Mean (SD) | −13.3 (23.37) | −3.0 (3.51) | −7.8 (16.54) |
| Median | 0.0 | 0.0 | 0.0 |
| Q1:Q3 | −23:0 | −7:0 | −7:0 |
| Min:Max | −60:0 | −7:0 | −60:0 |

TABLE 4-39
EORTC-QLQ-C30 Physical Functioning over Time in 2092-
Baseline to Week 28 (Full Analysis Set) (pt. 6)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| Physical Functioning | | | | | | |
| Day 113 | | | | | | |
| Percent change from baseline | | | | | | |
| n | 8 | | 9 | | 17 | |
| Mean (SD) | −13.7 | (23.85) | −3.1 | (3.77) | −8.1 | (16.89) |
| Median | 0.0 | | 0.0 | | 0.0 | |
| Q1:Q3 | −25:0 | | −7:0 | | −7:0 | |
| Min:Max | −60:0 | | −8:0 | | −60:0 | |
| Day 141 | | | | | | |
| Observed Value | | | | | | |
| n | 10 | | 11 | | 21 | |
| Mean (SD) | 82.0 | (18.87) | 93.9 | (8.14) | 88.3 | (15.19) |
| Median | 90.0 | | 100.0 | | 93.3 | |
| Q1:Q3 | 67:100 | | 87:100 | | 80:100 | |
| Min:Max | 47:100 | | 80:100 | | 47:100 | |
| Change from baseline | | | | | | |
| n | 8 | | 9 | | 17 | |
| Mean (SD) | −12.5 | (18.67) | 0.0 | (3.33) | −5.9 | (14.12) |

| Median | −6.7 | 0.0 | 0.0 |
|---|---|---|---|
| Q1:Q3 | −17:0 | 0:0 | −7:0 |
| Min:Max | −53:0 | −7:7 | −53:7 |

TABLE 4-40
EORTC-QLQ-C30 Physical Functioning over Time in 2092-
Baseline to Week 28 (Full Analysis Set) (pt. 7)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| Physical Functioning | | | | | | |
| Day 141 | | | | | | |
| Percent change from baseline | | | | | | |
| n | 8 | | 9 | | 17 | |
| Mean (SD) | −13.1 | (18.77) | −0.1 | (3.45) | −6.1 | (14.35) |
| Median | −6.9 | | 0.0 | | 0.0 | |
| Q1:Q3 | −19:0 | | 0:0 | | −7:0 | |
| Min:Max | −53:0 | | −7:7 | | −53:7 | |
| Day 169 | | | | | | |
| Observed Value | | | | | | |
| n | 11 | | 12 | | 23 | |
| Mean (SD) | 86.1 | (18.96) | 90.6 | (9.19) | 88.4 | (14.53) |
| Median | 93.3 | | 93.3 | | 93.3 | |
| Q1:Q3 | 73:100 | | 83:100 | | 80:100 | |
| Min:Max | 40:100 | | 73:100 | | 40:100 | |
| Change from baseline | | | | | | |
| n | 8 | | 9 | | 17 | |
| Mean (SD) | −12.5 | (20.30) | −2.2 | (4.71) | −7.1 | (14.81) |

| Median | −6.7 | 0.0 | 0.0 |
|---|---|---|---|
| Q1:Q3 | −13:0 | 0:0 | −7:0 |
| Min:Max | −60:0 | −13:0 | −60:0 |

TABLE 4-41
EORTC-QLQ-C30 Physical Functioning over Time in 2092-
Baseline to Week 28 (Full Analysis Set) (pt. 8)

| | Pozelimab Q2W + | | Pozelimab Q4W + | | | |
|---|---|---|---|---|---|---|
| Parameter (unit) | Cemdisiran | | Cemdisiran | | Total | |
| Visit | (N = 12) | | (N = 12) | | (N = 24) | |
| Physical Functioning | | | | | | |
| Day 169 | | | | | | |
| Percent change from baseline | | | | | | |
| n | 8 | | 9 | | 17 | |
| Mean (SD) | −13.0 | (20.27) | −2.2 | (4.71) | −7.3 | (14.90) |
| Median | −6.9 | | 0.0 | | 0.0 | |
| Q1:Q3 | −15:0 | | 0:0 | | −7:0 | |
| Min:Max | −60:0 | | −13:0 | | −60:0 | |
| Day 197 | | | | | | |
| Observed Value | | | | | | |
| n | 11 | | 12 | | 23 | |
| Mean (SD) | 90.9 | (16.67) | 95.0 | (5.77) | 93.0 | (12.14) |
| Median | 100.0 | | 96.7 | | 100.0 | |
| Q1:Q3 | 93:100 | | 90:100 | | 93:100 | |
| Min:Max | 47:100 | | 87:100 | | 47:100 | |
| Change from baseline | | | | | | |
| n | 8 | | 9 | | 17 | |
| Mean (SD) | −6.7 | (19.19) | −0.7 | (9.09) | −2.7 | (14.73) |

| Median | 0.0 | 0.0 | 0.0 |
|---|---|---|---|
| Q1:Q3 | −3:0 | 0:0 | 0:0 |
| Min:Max | −53:7 | −13:20 | −53:20 |

TABLE 4-42
EORTC-QLQ-C30 Physical Functioning over Time in 2092-
Baseline to Week 28 (Full Analysis Set) (pt. 9)

| | Pozelimab Q2W + | Pozelimab Q4W + | |
|---|---|---|---|
| Parameter (unit) | Cemdisiran | Cemdisiran | Total |
| Visit | (N = 12) | (N = 12) | (N = 24) |
| Physical Functioning | | | |
| Day 197 | | | |
| Percent change from baseline | | | |
| n | 8 | 9 | 17 |
| Mean (SD) | −6.6 (19.24) | 1.5 (10.59) | −2.3 (15.34) |
| Median | 0.0 | 0.0 | 0.0 |
| Q1:Q3 | −3:0 | 0:0 | 0:2 |
| Min:Max | −53:7 | −13:25 | −53:25 |

### Results (24 Randomized Patients Completed the OLTP, and 23 Entered the Optional OLEP)

At data cut-off, all 24 randomized patients completed the OLTP, and 23 entered the optional OLEP. No patient required treatment intensification.

At study baseline, patients were well controlled on Pozelimab monotherapy (Table 4-43).

TABLE 4-43
Baseline characteristics

| Characteristic | Arm 1 (n = 12) | | Arm 2 (n = 12) | |
|---|---|---|---|---|
| Age, years, mean (SD) | 53.2 | (16.4) | 41.4 | (16.9) |
| Sex, male, n (%) | 6 | (50.0) | 7 | (58.3) |
| Ethnicity, not Hispanic or Latino, n | 12 | (100.0) | 12 | (100.0) |
| (%) | | | | |
| Race, n (%) | | | | |
| Asian | 11 | (91.7) | 10 | (83.3) |
| White | 1 | (8.3) | 1 | (8.3) |
| Other | 0 | | 1 | (8.3) |
| PNH diagnosis age, years, | 45.0 | (32:50) | 30.0 | (23:49) |
| median (Q1:Q3) | | | | |
| Baseline LDH, U/L, mean (SD) | 237.9 | (41.1) | 237.0 | (44.4) |
| Baseline LDH, x ULN, mean (SD) | 0.79 | (0.18) | 0.79 | (0.17) |
| Hemoglobin, g/L, mean (SD) | 115.4 | (17.2) | 108.3 | (21.4)^{a} |
| eGFR, mL/min/1.73 m², mean | 104.1 | (21.9) | 117.8 | (18.2) |
| (SD) | | | | |
| Total bilirubin, μmol/L, mean (SD) | 22.8 | (12.3) | 31.5 | (15.6) |
| Haptoglobin, g/L, mean (SD) | 0.1 | (0.1) | 0.1 | (0.1) |
| Complement CH50, kIU/L, mean | 0.1 | (0.3) | 0.0 | (0.0) |
| (SD) | | | | |

^{a}n = 11.
Arm 1: Pozelimab 400 mg SC Q4W + Cemdisiran 200 mg SC Q4W.
Arm 2: Pozelimab 400 mg SC Q2W + Cemdisiran 200 mg SC Q4W.
CH50, total complement hemolysis assay; eGFR, estimated glomerular filtration rate; LDH, lactate dehydrogenase; PNH, paroxysmal nocturnal hemoglobinuria; Q, quartile; Q2W, every 2 weeks; Q4W, every 4 weeks; SC, subcutaneous; SD, standard deviation; ULN, upper limit of normal

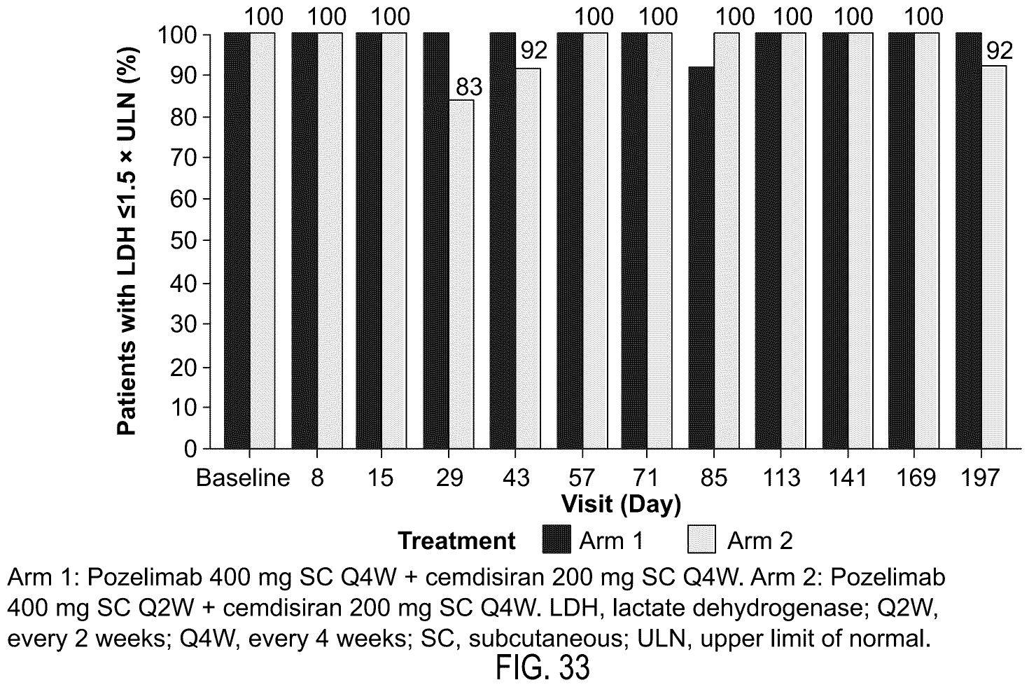

The majority of patients achieved an LDH≤1.5×ULN at each visit throughout the OLTP (FIG. 33), with most patients having an LDH value <1.0×ULN for most of the observation period.

Two patients (both from Arm 2) experienced an episode of moderate non-serious breakthrough hemolysis (one in association with a chlamydia infection, and one associated with gastroenteritis).

In total, four patients received blood transfusions from baseline through to Week 28 (Day 197; all patients from Arm 2). This includes the two patients with an event of breakthrough hemolysis (both patients continued to receive the combination therapy after receiving a blood transfusion).

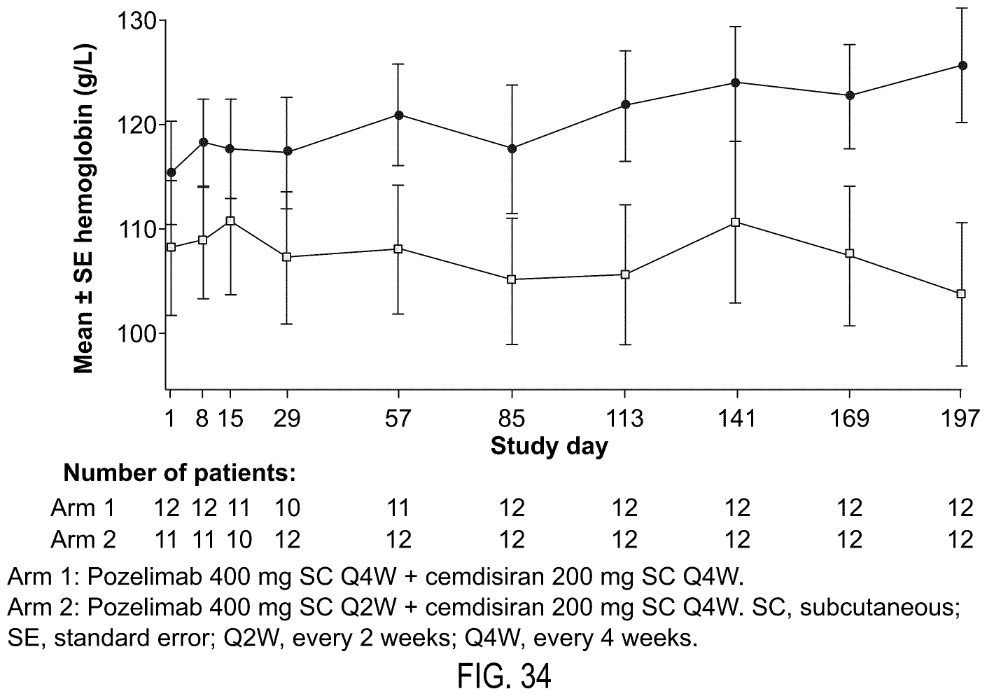

At the time of this primary analysis, 75.0% (18 of 24) of patients met the criteria for hemoglobin stabilization (i.e., did not receive a red blood cell transfusion and had no decrease in hemoglobin ≥2 g/dL; FIG. 34).

CH50, a measure of total complement hemolysis activity, remained fully suppressed at all post-baseline timepoints measured, including during the aforementioned breakthrough hemolysis events.

A total of 46 treatment-emergent adverse events (TEAEs) were reported in 16 patients (66.7%), none of which led to treatment discontinuation; 43 were of mild-to-moderate intensity.

Three severe TEAEs occurred in two patients: one patient had anemia, and another patient had two events (gastroenteritis in association with an event of breakthrough hemolysis).

Three patients in arm 2 had one serious TEAE each (COVID-19, upper respiratory tract infection, and the aforementioned gastroenteritis).

No serious or severe TEAEs were considered related to the study treatment, and all were resolved. There were no meningococcal infections, thrombotic events or deaths.

At the time of this primary analysis, Pozelimab and Cemdisiran combination treatment was generally well tolerated in patients with PNH who transitioned from Pozelimab monotherapy, regardless of the treatment arm.

The majority of patients maintained adequate control of hemolysis, most maintaining normalization of LDH (<1.0×ULN).

Most patients (75%) achieved hemoglobin stabilization during the OLTP.

These findings support the ongoing development of the combination of Pozelimab and Cemdisiran for the treatment of patients with PNH.

### Example 5: Clinical Trial Evaluating the Safety and Efficacy of Pozelimab and Cemdisiran Combination Therapy in Patients with Paroxysmal Nocturnal Hemoglobinuria (PNH) Who Switch from Eculizumab (R3918-PNH-20105)

PNH is an ultra-rare, acquired disorder caused by a mutation in the phosphatidylinositol glycan class A (PIGA) gene, which leads to impaired expression of complement-regulating proteins on the surface of hematopoietic cells. Clinical presentation of PNH includes hemolytic anemia, hemoglobinuria, and thrombosis. Complement component C5 inhibitors such as Eculizumab are part of the current standard of care for PNH patients; however, this is an intravenous treatment which relies on nurse administration. In addition, some patients experience an incomplete response to therapy, and may still experience breakthrough hemolytic events. Pozelimab and Cemdisiran are therapeutic agents with a subcutaneous maintenance regimen that may be self-administered. Both agents act together to inhibit terminal complement through complementary mechanisms of action. The efficacy and safety of the combination of Pozelimab and Cemdisiran is being evaluated in an ongoing, phase 2, open-label single-arm study in patients with PNH who switch from Eculizumab therapy (NCT04888507).

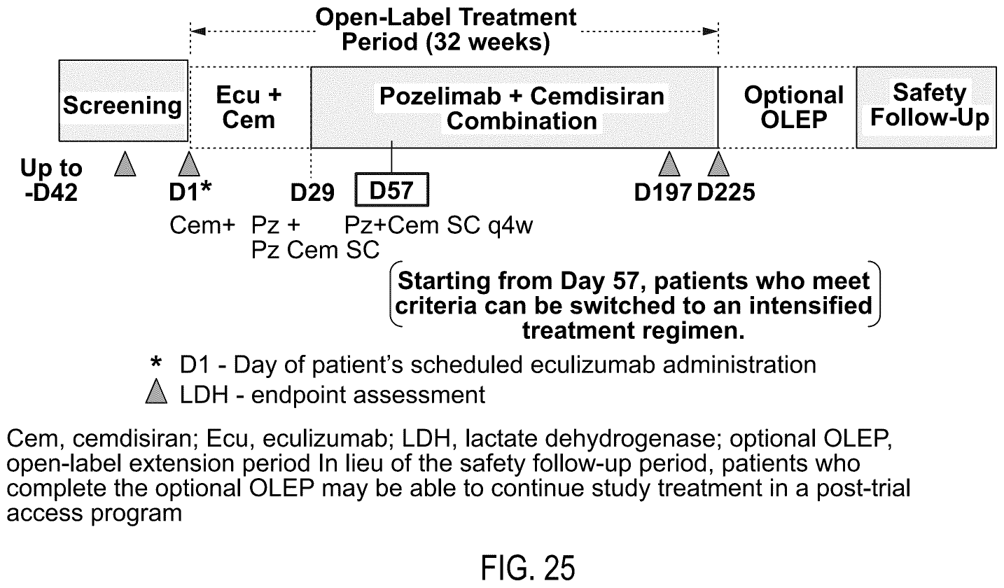

The study has 4 periods: a screening period of up to 42 days, a 32-week open label treatment period (OLTP (main study period), longer for patients who are switched to treatment intensification), an optional 52-week OLEP (Open-label extension period (an optional period)), and a 52-week post-treatment safety follow-up period. The fourth period begins when a patient completes or permanently discontinues study treatment (e.g., at the time of premature study drug discontinuation, at the completion of study treatment in the OLTP for patients who decline the optional OLEP, or at the completion of study treatment in the optional OLEP). See the study flow diagram of FIG. 25

Safety and efficacy results are presented herein. Baseline characteristics of subjects is set forth in Table 5-1.

TABLE 5-1
Baseline demographics and clinical characteristics

| | Pozelimab 400 mg Q4W + | |
|---|---|---|
| | cemdisiran 200 mg SC Q4W | |
| | (n = 6) | |
| Age at screening, years, median | 52.0 | (36:65) |
| (min:max) | | |
| Male sex, n (%) | 3 | (50.0) |
| Race, White, n (%) | 6 | (100) |
| BMI, kg/m², median (min:max) | 27.2 | (24.7:49.7) |
| Eculizumab dosage, n (%) | | |
| Standard (900 mg Q2W) | 4 | (66.7) |
| Non-standard (1200 or 1500 mg Q2W) | 2 | (33.3) |
| LDH,^{a} U/L, median (min:max) | 221.5 | (178.5:288.5) |
| Hemoglobin, g/L, median (min:max) | 110.0 | (97:142) |
| PNH RBC total,^{b} % | | |
| Mean (SD) | 64.1 | (36.9) |
| PNH PMN,^{b} % | | |
| Mean (SD) | 73.4 | (26.9) |
| PNH monocytes,^{b} % | | |
| Mean (SD) | 80.7 | (23.7) |
| Total bilirubin, μmol/L, median | 12.2 | (8.7:19.5) |
| (min:max) | | |
| Reticulocytes, 10\hat{ }9/L, median | 173.8 | (77.4:388.3) |
| (min:max) | | |
| eGFR, mL/min/1.73 m², median | 109.5 | (80:121) |
| (min:max) | | |
| Creatinine, μmol/L, median (min:max) | 57.5 | (35:88) |

^{a}Pre-treatment LDH values.
^{b}Three samples were missing from the central laboratory, so local laboratory pre-study PNH clone data were used.
BMI, body mass index; eGFR, estimated glomerular filtration rate; LDH, lactate dehydrogenase; PMN, polymorphonuclear neutrophils; PNH, paroxysmal nocturnal hemoglobinuria; Q2W, every 2 weeks; Q4W, every 4 weeks; RBC, red blood cell; SC, subcutaneous; SD, standard deviation.

### Inclusion Criteria

A patient must meet the following criteria to be eligible for inclusion in the study:
1. Male or female ≥18 years of age at the time of consent;
2. Diagnosis of paroxysmal nocturnal hemoglobinuria confirmed by a history of high-sensitivity flow cytometry from prior testing;
3. Treated with stable (i.e., no change in dose or frequency) Eculizumab therapy at the labeled dosing regimen or a higher dose and/or more frequently administered than labeled for at least 12 weeks prior to screening visit;
4. Provide informed consent signed by study patient;
5. Willing and able to comply with clinic/remote visits and study-related procedures;
6. Able to understand and complete study-related questionnaires.

### Exclusion Criteria

A patient who meets any of the following criteria will be excluded from the study:
1. History of bone marrow transplantation or receipt of an organ transplant;
2. Body weight <40 kg at screening;
3. Current plans for modification (initiation, discontinuation, or dose/dosing interval change) of the following background concomitant medications, as applicable, during screening and treatment period: erythropoietin, immunosuppressive drugs, corticosteroids, antithrombotic agents, anticoagulants, iron supplements, and folic acid;
4. Any use of complement inhibitor therapy other than Eculizumab in the 12 weeks prior to the screening visit or planned use during the study;
5. Any 2 of the following 3 abnormalities at the screening visit (one repeat measurement is allowed during screening period):
a. Peripheral blood absolute neutrophil count (ANC)<500/μL [<0.5×10⁹/L], or
b. Peripheral blood platelet count <20,000/μL, or
c. Peripheral blood reticulocyte count abnormality defined as <20,000/μL or <1%;
6. Known hypocellular bone marrow based on a history of reduced age-adjusted bone marrow cellularity and/or bone marrow cellularity <25%;
7. No documented meningococcal vaccination within 5 years prior to screening visit unless it is documented that vaccination has been administered during the screening period and prior to initiation of study treatment;
8. Unable to take antibiotics for meningococcal prophylaxis, if required by local standard of care;
9. Any active, ongoing infection or a recent infection requiring ongoing systemic treatment with antibiotics, antivirals, or antifungals within 2 weeks of screening or during the screening period;
10. Documented history of systemic fungal disease or unresolved tuberculosis (TB), or evidence of active or latent tuberculosis infection (LTBI) during screening period. Assessment for active TB and LTBI should be according with local practice or guidelines, including those pertaining to risk assessment, and the use of tuberculin skin test or T-cell interferon gamma release assay;
11. Positive hepatitis B surface antigen or hepatitis C virus RNA during screening.
   Note: Cases with unclear interpretation should be discussed with the medical monitor;
12. History of human immunodeficiency virus (HIV) infection;
13. Documented* positive polymerase chain reaction (PCR) or equivalent test based on regional recommendations for COVID-19 or suspected SARS-CoV-2 infection, and:
a. Have not recovered from COVID-19 (i.e., all COVID-19-related symptoms and major clinical findings which can potentially affect the safety of the patient have not been resolved); and
b. Did not have 2 negative results from nucleic acid amplification (PCR) test or equivalent test based on regional recommendations for COVID-19 to confirm that the patient is negative for SARS-CoV-2 or, if COVID-19 PCR (or equivalent) testing is not feasible, at least 3 months have transpired since the initial diagnosis.
*Note: Screening for COVID-19 will not be performed as part of eligibility assessments for this study;
14. Known hereditary complement deficiency;
15. Documented history of active, uncontrolled, ongoing systemic autoimmune diseases;
16. Documented history of liver cirrhosis or patients with liver disease with evidence of current, impaired liver function, or patients with ALT or AST (unrelated to PNH) greater than 3×ULN at the screening visit (one repeat assessment allowed during screening);
17. Patients with an eGFR of <30 mL/min/1.73 m² (according to Chronic Kidney Disease-Epidemiology Collaboration equation 2009 [CKD-EPI]) at screening visit (one repeat assessment allowed during screening);
18. Recent, unstable medical conditions, excluding PNH and PNH-related complications, within the past 3 months prior to screening visit (e.g., myocardial infarction, congestive heart failure with New York Heart Association Class III or IV, serious uncontrolled cardiac arrhythmia, cerebrovascular accident, active gastrointestinal bleed);
19. Anticipated need for major surgery during the study;
20. History of cancer within the past 5 years, except for adequately treated basal cell skin cancer, squamous cell skin cancer, or in situ cervical cancer;
21. Known hypersensitivity to Pozelimab, Cemdisiran, or to any components of the respective formulations;
22. Known or documented functional or anatomic asplenia;
23. Any clinically significant abnormality identified at the time of screening that in the judgment of the investigator or any sub-investigator would preclude safe completion of the study or constrain endpoints assessment such as major systemic diseases, or patients with short life expectancy;
24. Considered for any reason, including but not limited to the following:
   Deemed unable to meet specific protocol requirements, such as scheduled visits,
   Deemed unable to administer or tolerate long-term injections,
   Presence of any other conditions (e.g., geographic, social), actual or anticipated, that the investigator feels would restrict or limit the patient's participation for the duration of the study,
   Part of a vulnerable population such as the institutionalized;
25. Members of the clinical site study team and/or his/her immediate family, unless prior approval granted;
26. Pregnant or breastfeeding women;
27. Women of childbearing potential (WOCBP)* who are unwilling to practice highly effective contraception prior to the initial dose/start of the first treatment, during the study, and for at least 52 weeks after the last dose. Highly effective contraceptive measures include:
a. stable use of combined (estrogen- and progestogen-containing) hormonal contraception (oral, intravaginal, transdermal) or progestogen-only hormonal contraception (oral, injectable, implantable) associated with inhibition of ovulation initiated 2 or more menstrual cycles prior to screening;
b. intrauterine device (IUD); intrauterine hormone-releasing system (IUS);
c. bilateral tubal ligation;
d. vasectomized partner (provided that the male vasectomized partner is the sole sexual partner of the WOCBP study participant and that the vasectomized partner has obtained medical assessment of surgical success for the procedure); and/or
e. sexual abstinence†, ‡.
* WOCBP are defined as women who are fertile following menarche until becoming postmenopausal, unless permanently sterile. Permanent sterilization methods include hysterectomy, bilateral salpingectomy, and bilateral oophorectomy. A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance (CTFG. 2020. Recommendations related to contraception and pregnancy testing in clinical trials. Vol. v1.1. Clinical Trials Facilitation and Coordination Group). Pregnancy testing and contraception are not required for women with documented hysterectomy or tubal ligation.
†Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient.
‡Periodic abstinence (calendar, symptothermal, post-ovulation methods), withdrawal (coitus interruptus), spermicides only, condom use and lactational amenorrhea method (LAM) are not acceptable methods of contraception. Female condom and male condom should not be used together;
28. Participation in another interventional clinical study or use of any experimental therapy within 30 days before screening visit or within 5 half-lives of that investigational product, whichever is greater;
29. Patients with a history of significant multiple intolerability and/or severe allergies (including latex gloves) or patients who have had an anaphylactic reaction or significant multiple intolerability to prescription or non-prescription drugs;
30. Patients who have not been vaccinated against Streptococcus pneumoniae and Haemophilus influenza type B if recommended by current national/local vaccination guidelines. (Note that patients who were not previously vaccinated shall receive these vaccinations during screening, if recommended by current national/local vaccination guidelines).

### Methods

This phase 2 trial consists of a screening period (up to 42 days), a 32-week open-label treatment period (OLTP), an optional 52-week open-label extension period, and a 52-week post-treatment safety follow-up period. Patients transition from Eculizumab therapy to the combination of Pozelimab and Cemdisiran. An IV loading dose of Pozelimab is administered prior to the first dose of subcutaneous Pozelimab. The combination of subcutaneous Pozelimab and subcutaneous Cemdisiran are administered every 4 weeks.

Adequate control of hemolysis is defined as lactate dehydrogenase (LDH)≤1.5×ULN. Breakthrough hemolysis is defined as an increase in LDH (LDH≥2×ULN if pre-treatment LDH was <1.5×ULN, or LDH≥2×ULN after initial achievement of LDH≤1.5×ULN if pre-treatment LDH was >1.5×ULN) with concomitant signs or symptoms associated with hemolysis.

For the first approximately 2 weeks, patients remain on Eculizumab background treatment at their usual dose/frequency, and Cemdisiran alone is introduced. Lead-in Cemdisiran treatment and background concomitant treatment with Eculizumab Day 1 (the day of patient's scheduled Eculizumab administration): Cemdisiran 200 mg SC and Eculizumab ≥900 mg IV (at the patient's usual dose). Note: Eculizumab may be administered up to 2 days after Cemdisiran if not administered with Cemdisiran on day 1. Day 15: For patients on Eculizumab Q14 days (labeled dose regimen): day 15 (±2 days), i.e., day 13 to day 17; For patients on Eculizumab more frequently than Q14 days: patients are to be dosed within 2 days of their usual planned dose.

The following dosage regimen of Pozelimab/Cemdisiran combination therapy is administered during the switch from Eculizumab treatment: Pozelimab/Cemdisiran combination treatment: Day 29 (week 4): Pozelimab 60 mg/kg IV loading dose, followed (after a delay of at least 30 minutes) by Pozelimab 400 mg SC and Cemdisiran 200 mg SC; Day 57 (week 8) to day 197 (week 28): Pozelimab 400 mg SC and Cemdisiran 200 mg SC Q4W maintenance regimen (±7 days).

While the overall goal of the proposed dosing regimen is to prevent hemolysis, the transition period of the combination treatment initiation is also designed to mitigate the potential for the formation of large drug-target-drug (DTD) immune complexes of eculizumab-C5-pozelimab during the treatment switch. A lead-in cemdisiran dose plus a loading dose of pozelimab should minimize large DTD immune complex formation. A previous clinical study reported adverse reactions (e.g., serum sickness-like reactions, skin rash), upon switching from eculizumab to another anti-C5 mAb, crovalimab, attributed to the formation of immune complexes between C5 and the 2 non-competing C5 mAbs (Röth et al., Ravulizumab (ALXN1210) in patients with paroxysmal nocturnal hemoglobinuria: results of 2 phase 1b/2 studies. Blood Adv 2018; 2(17):2176-85). See Nishidate et al., Validation of a method to analyze size distribution of crovalimab-complement C5-eculizumab complexes in human serum. Bioanalysis. 2022 July; 14(13):935-947-Epub 2022 Jul. 29; Nishimura et al., Mitigating Drug-Target-Drug Complexes in Patients With Paroxysmal Nocturnal Hemoglobinuria Who Switch C5 Inhibitors. Clin Pharmacol Ther. 2023 April; 113(4):904-915-Epub 2023 Feb. 12. Similarly, pozelimab has been shown to bind C5 non-competitively with eculizumab (R3918-PH-19074), and thus has the potential to form higher-order immune complexes in the presence of eculizumab and C5. In vitro studies were conducted to simulate conditions that may occur when pozelimab is administered to a patient previously dosed with eculizumab. Neither pozelimab nor antibodies having the Eculizumab amino acid sequence (Eculizumab*) individually formed higher order multimers larger than a 1:2 mAb:C5 complex. Pozelimab was added to pre-formed eculizumab*:C5 complexes under conditions of excess pozelimab (5:1:1 pozelimab: eculizumab*:C5) and equimolar amounts of total mAb to C5 (1:1:2 pozelimab: eculizumab*:C5). Under conditions of pozelimab excess, the majority of complexes observed (˜86%) were either trimeric or pentameric (2:1 or 3:2 mAb:C5 molar ratios, respectively), with the remainder comprising large DTD immune complexes. At an equimolar ratio of total mAb and C5, the majority of the samples (˜86%) consisted of heterogeneous large DTD immune complexes larger than pentamers. In summary, while eculizumab* and pozelimab in combination were able to form heteromeric complexes with C5, the presence of excess pozelimab reduced the formation of higher-order immune complexes relative to conditions where total mAb and C5 were present at equimolar concentrations. In the current study, a 4-week lead-in period with a dose of cemdisiran 200 mg SC is provided. This is expected to reduce C5 production and consequently lower the total C5 (eculizumab*-C5 complexes plus free C5) level (about 86% reduction of total C5 on day 29 compared to day 1), before the initiation of the pozelimab/cemdisiran combination treatment. Consequently, the potential for the formation of the large DTD immune complexes should be significantly reduced. To further minimize the risk of formation of large DTD immune complexes upon initiating pozelimab/cemdisiran combination treatment, a loading dose of pozelimab 60 mg/kg IV is provided. Based on reported mean trough concentrations of eculizumab of 97 mg/L (Soliris, 2021) and predicted pozelimab concentration over time, the molar concentrations of pozelimab should be in excess of eculizumab following the IV loading dose with an estimated ratio of approximately 17:1. A lead-in cemdisiran dose plus a loading dose of pozelimab is considered the best solution to address this concern and provide rapid and sustained maximum C5 inhibition after the initiation of the combination treatment and throughout the dosing intervals. In addition to being part of the approach to mitigate the potential for the formation of large DTD immune complexes, the IV loading dose of pozelimab 60 mg/kg should ensure rapid and complete inhibition of C5 to avoid any breakthrough hemolysis that could occur during the treatment transition. Based on simulations, the dosage regimen of the IV loading dose followed by the SC maintenance dose of pozelimab/cemdisiran 400 mg/200 mg Q4W starting on day 29 should result in rapid and sustained suppression of C5 to biologically inactive levels.

See the schedule of the various doses administered in Table 5-2-5-5 below.

TABLE 5-2
Schedule of Events for Open-Label Treatment Period

| Study Procedure¹ | | | | | | | | | | | | | | |
|---|---|---|---|---|---|---|---|---|---|---|---|---|---|---|
| Screening Period | | Open-Label Treatment Period | | | | | | | | | | | | |
| Visit # | | | | | | | | | | | | | | |
| V1a | V1b² | V2 | V3 | V4 | V5 | V6 | V7 | V8 | V9 | V10 | V11 | V12 | V13 | EOT V14 |

| Week | | | | | | | | | | | | | |
|---|---|---|---|---|---|---|---|---|---|---|---|---|---|
| Up to −6 | 0 | 1 | 212 | 4 | 6 | 8 | 10 | 12 | 16 | 20 | 24 | 28 | 32 |
| Day | | | | | | | | | | | | | |
| Up to −42 | 1 | 8 | 15 | 29 | 43 | 57 | 71 | 85 | 113 | 141 | 169 | 197 | 225 |

Window (day)

| | | | | | | | | | | | |
|---|---|---|---|---|---|---|---|---|---|---|---|
| ±2 | ±2 | ±3 | ±3 | ±3 | ±3 | ±3 | ±7 | ±7 | ±7 | ±7 | ±7 |

Screening/Baseline:

| | | | | | | | | | | | | | | | |
|---|---|---|---|---|---|---|---|---|---|---|---|---|---|---|---|
| Inclusion/exclusion criteria | X | X | X | | | | | | | | | | | | |
| Informed consent | X | | | | | | | | | | | | | | |
| Informed consent for OLEP | X | | | | | | | | | | | | | | |
| Informed consent for FBR (optional) | X | | | | | | | | | | | | | | |
| Informed consent for genomic testing | X | | | | | | | | | | | | | | |
| (optional) | | | | | | | | | | | | | | | |
| Medical history³ | X | | | | | | | | | | | | | | |
| Prior medications⁴ | X | X | | | | | | | | | | | | | |
| Demographics | X | | | | | | | | | | | | | | |
| Height | X | | | | | | | | | | | | | | |
| Vaccination/re-vaccinate for Neisseria | X | | | | | | | | | | | | | | |
| meningitidis⁵ | | | | | | | | | | | | | | | |
| Vaccination against Streptococcus | X | | | | | | | | | | | | | | |
| pneumoniae and | | | | | | | | | | | | | | | |
| Haemophilus influenza type B (if needed) | | | | | | | | | | | | | | | |
| Tuberculosis history and assessment⁶ | X | | | | | | | | | | | | | | |
| Risk assessment for Neisseria gonorrhea7 | X | | | | | | | | | | | | | | |
| Patient safety card forNeisseria | | | X | X | X | X | X | X | X | X | X | X | X | X | X |
| meningitidis⁸ | | | | | | | | | | | | | | | |
| Enrollment | | | X | | | | | | | | | | | | |

Treatment:

| Administer Cemdisiran 200 mg SC Q4W^{9,11} | | X | | X | | X | | X | X | X | X | X | |
|---|---|---|---|---|---|---|---|---|---|---|---|---|---|
| Administer Pozelimab IV 60 mg/kg¹⁰ | | | | X | | | | | | | | | |
| Administer Pozelimab 400 mg SC Q4W¹¹ | | | | X | | X | | X | X | X | X | X | |
| Administer Eculizumab¹² | | X | X | | | | | | | | | | |
| Injection Training/patient instructions, if | | | | X | X | X | X | X | X | X | X | X | |
| needed¹³ | | | | | | | | | | | | | |
| Patient diary¹⁴ | | | | | | X | X | X | X | X | X | X | X |
| Concomitant meds and procedure | | <------------------------------------------------------------X----------------------------------------> | | | | | | | | | | | |
| Transfusion record update | ←----------------------------------------------------------------X-----------------------------------------------------> | | | | | | | | | | | | |
| Antibiotics prophylaxis (recommended)¹⁵ | | ←---------------------------------------------------------------------X-----------------------------> | | | | | | | | | | | |
| Revaccination against meningococcal | ←----------------------------------------------------------------X--------------------------------------------------> | | | | | | | | | | | | |
| infection (if needed) | | | | | | | | | | | | | |

Clinical Outcome Assessments:

| | | | | | | | | | | | | | | |
|---|---|---|---|---|---|---|---|---|---|---|---|---|---|---|
| FACIT-Fatigue | | X | | X | X | | X | | X | X | X | X | X | X |
| EORTC-QLQ-30 | | X | | X | X | | X | | X | X | X | X | X | X |
| TSQM | | X | | X | X | | X | | X | X | X | X | X | X |
| PNH symptom-specific questionnaire | X | X | X | X | X | X | X | X | X | X | X | X | X | X |
| (daily)¹⁶ | | | | | | | | | | | | | | |
| PGIS | | X | | | X | | | | X | | X | X | | X |
| PGIC | | | | | X | | | | X | | X | X | | X |

Safety and Anthropometric:

| | | | | | | | | | | |
|---|---|---|---|---|---|---|---|---|---|---|
| Body weight | X | | | X | X | X | X | X | X | X |
| Vital signs | X | X | X | X | X | X | X | X | X | X |
| Physical examination | X | X | | X | | X | | | | X |
| Electrocardiogram | X | | | | | | | | X | X |

| | |
|---|---|
| Adverse events | ←--------------------------------------------------------------------X--------------------------------------------------> |
| Breakthrough hemolysis assessment¹⁷ | ←-------------------------------------------------------------------X--------------------------------------------------> |

Laboratory Testing¹⁸:

| | | | | | | | | | | | | | | | |
|---|---|---|---|---|---|---|---|---|---|---|---|---|---|---|---|
| Titers to measure N. meningitidis (only if | X | | | | | | | | | | | | | | |
| required per local practice/regulations) | | | | | | | | | | | | | | | |
| Hematology | X | X² | X | X | X | X | | X | | X | | X | X | X | X |
| Coagulation panel | X | X² | X | X | X | X | X | X | X | X | X | X | X | X | X |
| Chemistry, including LDH¹⁹ | X | X² | X | X | X | X | X | X | X | X | X | X | X | X | X |
| Hepatitis B and C testing | X | | | | | | | | | | | | | | |
| Pregnancy test (applicable patients)²⁰ | X | | X | | | X | | X | | X | X | X | X | X | X |
| Urinalysis | X | | X | X | X | X | | X | | X | X | X | X | | X |

Pharmacokinetics, total C5, and Immunogenicity Sampling:

| | | | | | | | | | | | |
|---|---|---|---|---|---|---|---|---|---|---|---|
| Blood samples for conc. of | | | | X | X | X | X | X | X | X | X |
| Pozelimab²¹ | | | | | | | | | | | |
| Blood samples for conc. of | X | | | | | X | | | | X | |
| Cemdisiran and | | | | | | | | | | | |
| metabolites²² | | | | | | | | | | | |
| Blood samples for conc. of | X | | X | X | X | X | X | X | X | | X |
| Eculizumab²¹ | | | | | | | | | | | |
| Blood samples for conc. of | X | X | X | X | X | X | X | X | X | X | X |
| total C5²¹ | | | | | | | | | | | |
| Blood samples for | X | | | | | | X | | | | X |
| immunogenicity of | | | | | | | | | | | |
| Pozelimab²³ | | | | | | | | | | | |
| Blood samples for | X | | | | | X | | | | X | |
| immunogenicity of | | | | | | | | | | | |
| Cemdisiran²³ | | | | | | | | | | | |

Biomarkers:

| | | | | | | | | | | | | |
|---|---|---|---|---|---|---|---|---|---|---|---|---|
| Free hemoglobin | | X | | | X | X | X | X | X | X | X | X |
| Haptoglobin | | X | | | X | | X | | | | | X |
| Complement hemolytic | | X | X | X | X | X | X | X | X | X | X | X |
| assay (serum CH50)²¹ | | | | | | | | | | | | |
| Complement hemolytic | | X | X | X | X | X | X | X | X | X | X | X |
| assay (serum AH50)²¹ | | | | | | | | | | | | |
| sC5b-9 (plasma) | | X | X | X | X | X | X | X | X | X | X | X |
| PNH erythrocyte cells | X | | | | | X | | | | | | X |
| PNH granulocyte cells | X | | | | | X | | | | | | X |

Optional pharmacogenomics and future biomedical research:

| | | | | | | |
|---|---|---|---|---|---|---|
| Future research serum | X | X | X | X | X | X |
| and plasma (optional)²⁴ | | | | | | |
| Whole blood sample for | X | | | | | |
| DNA isolation (optional)²⁵ | | | | | | |
| Whole blood RNA sample | X | | | | | X |
| (optional) | | | | | | |

Footnotes
¹Study procedures: when multiple procedures are performed on the same day, the sequence of procedures is as follows: COA assessments, ECG and/or vital signs, blood collection, study treatment administration, and any pre-specified post-dose sample collection.
²Screening visit 1b can be combined with visit 1a, if LDH can be obtained one day before or on the day of Eculizumab administration. Visit 1b and additional interim visits may also be needed for repeat blood collection, vaccination, etc.
³Medical history: transfusions, breakthrough hemolysis history, and laboratory parameters for measurement of hemolysis (such as LDH, bilirubin, haptoglobin, reticulocyte count, and hemoglobin) should be obtained for the past 1 year, if possible. Prior history at any time of thrombosis and Neisseria infections will be collected if feasible. Ongoing PNH symptoms and signs will also be collected.
⁴Prior medications: including detailed Eculizumab administration history (past 26 weeks) and N. meningitidis vaccination (past 5 years); all other prior medications 12 weeks prior to screening.
⁵Patients will require administration with meningococcal vaccination unless documentation is provided of prior immunization in the past 5 years prior to screening, or less than 5 years if required according to national vaccination guidelines for vaccination use with complement inhibitors or local practice. For patients who require administration with meningococcal vaccination(s) during the screening period, administration should occur preferably at least 2 weeks prior to day 1, or at another time point according to local practice or national guidelines.
⁶Tuberculosis history and assessment. Screening by tuberculin skin test or T-cell interferon gamma release assay may be performed according to local practice or guidelines at the discretion of the investigator.
7A risk factor assessment for N. gonorrhea is recommended, and counseling is advised for at-risk patients.
⁸Patient safety card: provide the patient safety card for N. meningitidis infection to the patient on day 1 or any other visit when needed. Site should review the instructions on the safety card with the patient at each visit.
⁹Cemdisiran administration: the first day of dosing of Cemdisiran will take place at the patient's usual schedule of administration for Eculizumab.
¹⁰Pozelimab IV administration: administration at day 29 should precede SC administration. After completion of IV administration, the patient should be observed for at least 30 minutes and if no clinical concern, then SC administration of the combination should proceed. Patients should be monitored for at least another 30 minutes after the first SC dosing.
¹¹The SC doses of Pozelimab and Cemdisiran should be given Q4W (every 28 days) starting at day 29 (week 4). From day 57 (week 8) onward, Cemdisiran and Pozelimab SC administration may either be continued by the site personnel or another healthcare professional at the patient's home, or administration by the patient or designated person at the patient's preferred location after adequate training. The final SC dosing of the combination during the OLTP is at week 28. During the Q4W dose administration interval starting at day 57, the dose of Cemdisiran and Pozelimab SC should be given on the day of the corresponding study visit whenever possible. Study treatment administration should always be the last procedure after all blood sample collection and study assessments have been completed unless otherwise specified. If Pozelimab or Cemdisiran cannot be administered on the day of the corresponding study visit, the combination may be administered up to 7 days before or up to 7 days after the planned dosing date, provided that the dosing takes place after the corresponding study visit has been completed. For example, the day 57 (week 8) visit can take place from day 54 to day 60 given the visit window. The corresponding dose of Pozelimab and Cemdisiran can be given from day 54 to day 64, but only after the week 8 visit assessments have been performed. Similarly, the day 113 (week 16) visit can take place from day 106 to day 120 given the visit window. The corresponding dose of Pozelimab and Cemdisiran can be given from day 106 to day 120, but only after the week 16 visit assessments have been performed. Pozelimab and Cemdisiran should be administered on the same day whenever possible. Care must be taken to coordinate dosing for visits where a post-dose sample is collected to measure the concentration of Cemdisiran and its metabolites.
¹²Eculizumab administration: continue patient's Eculizumab administration at the usual dose and dosing interval. Administration of Eculizumab at day 1 (when first dose of Cemdisiran is administered) may occur up to 2 days later.
Note:
the week 2 visit should be scheduled relative to the patient's typical dosing frequency.
For patients taking Eculizumab with a frequency of:
Every 12 days
The visit should be scheduled on day 13 (±2 days).
Every 13 days
The visit should be scheduled on day 14 (±2 days).
Every 14 days
The visit should be scheduled on day 15 (±2 days).
The dose of Eculizumab should be administered according to the usual dose frequency and must be dosed on or after the visit and corresponding assessments have been performed.
¹³Injection training will be provided to patients who desire self-injection or injection by a designated person. Site staff should observe patient's self-injection or injection by a designated person and confirm adequacy. Patient instruction materials will be provided.
¹⁴If needed, based on patient self-administration/administration by a designated person, the patient will complete a diary for recording data on study treatment administration starting at the day 57 visit or a subsequent visit. If patient diary is provided to the patient, then it should be reviewed at each clinic visit and data collected into the case report forms (CRFs). On the final visit, the diary should be collected by the site.
¹⁵Daily oral antibiotic prophylaxis against N. meningitidis is recommended starting on the first day of dosing with study treatment and continuing until 52 weeks after discontinuation of Pozelimab/Cemdisiran. If vaccination for N. meningitidis occurs less than 2 weeks prior to day 1, then antibiotic prophylaxis must be administered for at least 2 weeks from the time of vaccination.
¹⁶Patients will complete the PNH Symptom-Specific Questionnaire on a daily basis for at least 14 days prior to the day 1 visit. Patients should try to complete the PNH Symptom-Specific Questionnaire at the same time each day whenever possible.
¹⁷Breakthrough hemolysis assessment: if a patient is suspected of having a breakthrough hemolysis event, then in addition to the required laboratory collection, additional samples for coagulation parameters, chemistry, hematology, reticulocyte count, D-dimer, total C5, CH50, ADA (against Pozelimab), and drug concentrations of Pozelimab and Eculizumab will be collected unless already noted in the Schedule of Events for that visit. If the suspected event does not occur at a scheduled visit, then an unscheduled visit should occur with an evaluation of the patient and collection of coagulation parameters, chemistry, hematology, reticulocyte count, total C5, CH50 and drug concentrations of Pozelimab and Eculizumab.
¹⁸During lab collection, handling, and processing, the same methodology will be applied across study visits, as best as possible, to preserve the quality of sample and avoid hemolysis. The coagulation blood sample (tube) must always be collected first, followed by the blood chemistry sample (tube). If the investigator or sponsor suspects that the lab result is not an accurate reflection of the patient's condition, the lab sample should be repeated.
¹⁹Serum LDH, CRP, and bilirubin will be assessed as part of the blood chemistry analysis. The coagulation blood sample (tube) must always be collected first, followed by the blood chemistry sample (tube). During screening, obtain chemistry including LDH prior to Eculizumab administration on the day of (or if not possible, one day before) Eculizumab administration. On day 1 and all subsequent visits, obtain chemistry including LDH prior to any study treatment administration.
²⁰Pregnancy test for WOCBP: a serum test will be done at the screening visit and a urine test will be done at all other visits indicated. Any positive urine test should be confirmed with a serum pregnancy test.
²¹Blood sample collection for concentrations of Pozelimab, Eculizumab, total C5, CH50 (efficacy endpoint), and AH50: obtain samples prior to any study drug administration (pre-dose). On day 29, obtain blood samples prior to IV administration of Pozelimab and also within 15 minutes after the end of the IV infusion.
²²Blood samples for concentrations of Cemdisiran and its metabolites will be collected prior to any study treatment administration (pre-dose) and at 2 to 6 hours post-Cemdisiran administration. The post-dose sample should be carefully coordinated with the dosing of Cemdisiran and may be collected at the clinic or by a visiting healthcare professional.
²³Blood samples for immunogenicity will be collected before the administration of any study drug (pre-dose). At the visits where immunogenicity samples are to be taken, the sample should be collected with the drug concentration sample. In the event of suspected SAEs, such as anaphylaxis or hypersensitivity, additional samples for drug concentration and immunogenicity may be collected at or near the event.
²⁴Future research serum and plasma (optional): samples should be collected, as permitted by patient consent and local regulatory policies. They may be stored for up to 15 years or as permitted by local regulatory policies, whichever is shorter, for future biomedical research.
²⁵Whole blood samples (optional) for DNA extraction should be collected on day 1 (pre-dose) but can be collected at a later study visit.

TABLE 5-3
Schedule of Events for Treatment Period (for Patients on Intensified Treatment in the OLTP)

| Study Procedure¹ | | | | | | | | | | | | |
|---|---|---|---|---|---|---|---|---|---|---|---|---|
| Intensified Treatment Period in the OLTP | | | | | | | | | | | | |
| Visit # | | | | | | | | | | | | |
| RV1 | RV2 | RV3 | RV4 | RV5 | RV6 | RV7 | RV8 | RV9 | RV10 | RV11 | RV12 | EOT RV13 |
| Week | | | | | | | | | | | | |
| 0r | 1r | 2r | 4r | 6r | 8r | 10r | 12r | 16r | 20r | 24r | 28r | 32r |
| Day | | | | | | | | | | | | |
| 1r | 8r | 15r | 29r | 43r | 57r | 71r | 85r | 113r | 141r | 169r | 197r | 225r |

Window (day)

| | | | | | | | | | | | |
|---|---|---|---|---|---|---|---|---|---|---|---|
| ±2 | ±2 | ±3 | ±3 | ±3 | ±3 | ±3 | −7/+3 | −7/+3 | −7/+3 | −7/+3 | −7/+3 |

Treatment:²

| Administer Pozelimab IV 30 mg/kg | X | | | | | | | | | | | |
|---|---|---|---|---|---|---|---|---|---|---|---|---|
| Administer Pozelimab 400 mg SC Q2W³ | X | | X | X | X | X | X | X | X | X | X | X3 |
| Administer Cemdisiran 200 mg SC Q4W³ | X | | | X | | X | | X | X | X | X | X |
| Injection Training/patient instructions, if | X | X | X | X | X | X | X | X | X | X | X | X |
| needed⁴ | | | | | | | | | | | | |
| Patient diary⁵ | X | X | X | X | X | X | X | X | X | X | X | X |
| Concomitant meds and procedures | ←--------------------------------------------------------------------X---------------------------------------------------> | | | | | | | | | | | |
| Transfusion record update | ←--------------------------------------------------------------------X---------------------------------------------------> | | | | | | | | | | | |
| Antibiotics prophylaxis (recommended)⁶ | ←---------------------------------------------------------------------X-----------------------------------------------> | | | | | | | | | | | |
| Re-vaccination against meningococcal | ←--------------------------------------------------------------------X---------------------------------------------------> | | | | | | | | | | | |
| infection (if needed) | | | | | | | | | | | | |

Clinical Outcome Assessments:

| | | | | | | | | | | | | | |
|---|---|---|---|---|---|---|---|---|---|---|---|---|---|
| FACIT-Fatigue | X | | X | X | | X | | X | X | X | X | X | X |
| EORTC-QLQ-30 | X | | X | X | | X | | X | X | X | X | X | X |
| TSQM | X | | X | X | | X | | X | X | X | X | X | X |
| PNH symptom-specific questionnaire | X | X | X | X | X | X | X | X | X | X | X | X | X |
| (daily)⁷ | | | | | | | | | | | | | |
| PGIS | X | | | X | | | | X | | X | X | | X |
| PGIC | | | | X | | | | X | | X | X | | X |

Safety and Anthropometric:

| Patient safety card for Neisseria | ←--------------------------------------------------------------------X---------------------------------------------------> | | | | | | | | |
|---|---|---|---|---|---|---|---|---|---|
| meningitidis⁸ | | | | | | | | | |
| Body weight | X | | X | X | X | X | X | X | X |
| Vital signs | X | X | X | X | X | X | X | X | X |
| Physical examination | X | | X | | X | | | | X |
| Electrocardiogram | | | | | | | | X | X |

| | |
|---|---|
| Adverse events | ←--------------------------------------------------------------------X---------------------------------------------------> |
| Breakthrough hemolysis assessment⁹ | ←----------------------------------------------------------------X---------------------------------------------------> |

Laboratory Testing:

| | | | | | | | | | | | | | |
|---|---|---|---|---|---|---|---|---|---|---|---|---|---|
| Titers to measure N. meningitidis (only if | X | | | | | | | | | | | | |
| required per local practice/regulations) | | | | | | | | | | | | | |
| Hematology | X | X | X | X | | X | | X | X | X | X | X | X |
| Coagulation panel | X | X | X | X | X | X | X | X | X | X | X | X | X |
| Chemistry including LDH¹¹ | X | X | X | X | X | X | X | X | X | X | X | X | X |
| Pregnancy test (applicable patients)¹² | X | | | X | | X | | X | X | X | X | X | X |
| Urinalysis | X | X | X | X | | X | | X | X | X | X | | X |

Pharmacokinetics, total C5, and Immunogenicity Sampling:

| | | | | | | | | | | | |
|---|---|---|---|---|---|---|---|---|---|---|---|
| Blood samples for conc. of Pozelimab¹³ | X | | X | X | X | X | X | X | X | X | X |
| Blood samples for conc. of Cemdisiran and | X | | | | | X | | | | X | |
| metabolites¹⁴ | | | | | | | | | | | |
| Blood samples for conc. of total C5¹³ | X | X | X | X | X | X | X | X | X | X | X |
| Blood samples for immunogenicity of | X | | | | | | X | | | | X |
| Pozelimab¹⁵ | | | | | | | | | | | |
| Blood samples for immunogenicity of | X | | | | | X | | | | X | |
| Cemdisiran¹⁵ | | | | | | | | | | | |

Biomarkers:

| | | | | | | | | | | | |
|---|---|---|---|---|---|---|---|---|---|---|---|
| Free hemoglobin | X | | | X | X | X | X | X | X | X | X |
| Haptoglobin | X | | | X | | X | | | | | X |
| Complement hemolytic assay (serum | X | X | X | X | X | X | X | X | X | X | X |
| CH50)¹³ | | | | | | | | | | | |
| Complement hemolytic assay (serum | X | X | X | X | X | X | X | X | X | X | X |
| AH50)¹³ | | | | | | | | | | | |
| sC5b-9 (plasma) | X | X | X | X | X | X | X | X | X | X | X |
| PNH erythrocyte cells | X | | | | X | | | | | | X |
| PNH granulocyte cells | X | | | | X | | | | | | X |

Footnotes
¹Study procedures: When multiple procedures are performed on the same day, the sequence of procedures is as follows: COA assessments, ECG and/or vital signs, blood collection, study treatment administration, and any pre-specified post-dose sample collection.
²Patients should be monitored for at least 30 minutes after completion of Pozelimab 30 mg/kg IV. Subsequent SC doses will be administered Q2W (Pozelimab) and Q4W (Cemdisiran) and may either be performed by the site personnel or another healthcare professional at the patient's home, or administered by the patient or by a designated person. For patients on intensified treatment in the OLTP, the final SC dose of Cemdisiran is at day 197r, week 28r, and the final SC dose of Pozelimab is at day 211r (week 30r).
³Pozelimab and Cemdisiran SC administration: the dose of Pozelimab SC should be given Q2W (every 14 days) and Cemdisiran should be given Q4W (every 28 days) and on the day of the corresponding study visit whenever possible and as applicable. Study treatment administration should always be the last procedure after all blood sample collection and study assessments have been completed unless otherwise specified. If administration of Pozelimab or Cemdisiran cannot be administered on the day of the corresponding study visit, the dose may be administered up to 3 days before or up to 3 days after the planned dosing date as long as the dosing takes place after the corresponding study visit has been completed. For example, the day 29r (week 4r) visit can take place from day 26r to day 32r given the visit window. The dose of Pozelimab and Cemdisiran therefore can be given from day 26r to day 32r, but only on or after the week 4r visit assessments have been performed. Similarly, the day 113r (week 16r) visit can take place from day 106r to day 116r given the visit window. The dose of Pozelimab and Cemdisiran can be given from day 110r to day 116r, but only on or after the week 16r visit assessments have been performed. Whenever possible, the dose of Cemdisiran should be administered on the same day as the Q4W dose of Pozelimab. The final dose of Cemdisiran is at week 28r and the final dose of Pozelimab is at week 30r.
⁴Injection training will be provided to patients who desire self-injection or injection by a designated person. The site should observe patient self-injection or injection by a designated person and confirm adequacy. Patient instruction materials will be provided.
⁵If needed, based on patient self-administration/administration by a designated person, the patient will complete a diary for recording data on study treatment administration. If the patient diary is provided to the patient, then it should be reviewed at each clinic visit and data collected into the CRFs. On the final visit, the diary should be collected by the site.
⁶Daily oral antibiotic prophylaxis against N. meningitidis is recommended until 52 weeks after discontinuation of Pozelimab/Cemdisiran.
⁷Patients should try to complete the PNH Symptom-Specific Questionnaire at the same time each day whenever possible.
⁸Patient safety card: the site should review the instructions on the safety card with the patient at each visit.
⁹Breakthrough hemolysis assessment: if a patient is suspected of having a breakthrough hemolysis event, then in addition to the required laboratory collection, additional samples for coagulation parameters, chemistry, hematology, reticulocyte count, D-dimer, total C5, CH50, ADA (against Pozelimab), and drug concentrations of Pozelimab will be collected unless already noted in the Schedule of Events for that visit. If the suspected event does not occur at a scheduled visit, then an unscheduled visit should occur with an evaluation of the patient and collection of coagulation parameters, chemistry, hematology, reticulocyte count, total C5, CH50 and drug concentrations of Pozelimab.
10. During lab collection, handling, and processing, the same methodology will be applied across study visits, as best as possible, to preserve the quality of the sample and avoid hemolysis. The coagulation blood sample (tube) must always be collected first, followed by the blood chemistry sample (tube).
¹¹Serum LDH, CRP, and bilirubin will be assessed as part of the blood chemistry analysis.
¹²Pregnancy test for WOCBP: a urine test will be done at all visits indicated.
¹³Blood sample collection for concentrations of Pozelimab, total C5, CH50 (efficacy endpoint), and AH50: obtain samples prior to any study drug administration (pre-dose). On day 1, obtain blood sample for Pozelimab concentration and total C5 prior to IV administration of Pozelimab and also within 15 minutes after the end of the IV infusion.
¹⁴Blood samples for concentrations of Cemdisiran and its metabolites will be collected prior to any study treatment administration (pre-dose) and at 2 to 6 hours post-Cemdisiran dosing. The post-dose sample should be carefully coordinated with the dosing of Cemdisiran and may be collected at the clinic or by a visiting healthcare professional.
¹⁵Blood samples for immunogenicity will be collected before the administration of any study drug (pre-dose). At the visits where immunogenicity samples are to be taken, the sample should be collected with the drug concentration sample. In the event of suspected SAEs, such as anaphylaxis or hypersensitivity, additional samples for drug concentration and immunogenicity may be collected at or near the event.

TABLE 5-4
Schedule of Events (Optional Open-Label Extension Period)

| Study Procedure (Visit)^{1,2} | | | | | | |
|---|---|---|---|---|---|---|
| Optional Open-Label Extension Period | | | | | | |
| OLEP-1³ | OLEP-2 | OLEP-3 | OLEP-4 | OLEP-5 | OLEP-6 | OLEP-7 |
| Week | | | | | | |
| 0e | 8e | 16e | 24e | 32e | 40e | 52e |
| Day | | | | | | |
| 1e | 57e | 113e | 169e | 225e | 281e | 365e |
| Window (day)⁴ | | | | | | |
| — | −7/+3 | −7/+3 | −7/+3 | −7/+3 | −7/+3 | −7/+3 |

Treatment⁵:

| Re-vaccination against | <_{——}_{——}_{——}_{——}_{——}_{——}_{——}_{——}_{——}X_{——}_{——}_{——}_{——}_{——}_{——}> | | | | | | |
|---|---|---|---|---|---|---|---|
| meningococcal infection (if | | | | | | | |
| needed) | | | | | | | |
| Pozelimab 400 mg SC Q2W or | X | X | X | X | X | X | X |
| Q4W⁶ | | | | | | | |
| Cemdisiran 200 mg SC Q4W⁶ | X | X | X | X | X | X | X |
| Injection training/patient | <_{——}_{——}_{——}_{——}_{——}_{——}_{——}_{——}_{——}X_{——}_{——}_{——}_{——}_{——}_{——}> | | | | | | |
| instructions (as needed)⁷ | | | | | | | |
| Patient diary⁸ | X | X | X | X | X | X | X |
| Antibiotics prophylaxis | <_{——}_{——}_{——}_{——}_{——}_{——}_{——}_{——}_{——}X_{——}_{——}_{——}_{——}_{——}_{——}> | | | | | | |
| (recommended)⁹ | | | | | | | |

Clinical Outcome Assessments:

| | | | |
|---|---|---|---|
| FACIT-Fatigue | X | X | X |
| EORTC-QLQ-C30 | X | X | X |
| PGIS | X | X | X |
| PGIC | X | X | X |

Safety and Anthropometric:

| | | | | | | | |
|---|---|---|---|---|---|---|---|
| Patient safety card for | X | X | X | X | X | X | X |
| Neisseria meningitidis¹⁰ | | | | | | | |
| Body weight | X | X | X | X | X | X | X |
| Vital signs | X | X | X | X | X | X | X |
| Physical examination | X | | X | | X | | X |
| Electrocardiogram | X | | | | | | X |
| Adverse events | X | X | X | X | X | X | X |
| Breakthrough hemolysis | X | X | X | X | X | X | X |
| assessment¹¹ | | | | | | | |
| Concomitant meds/treatments | X | X | X | X | X | X | X |
| Transfusion record update | X | X | X | X | X | X | X |

Laboratory Testing¹²:

| | | | | | | | |
|---|---|---|---|---|---|---|---|
| Titers to measure N. meningitidis | X | | | | | | |
| (only if required per local | | | | | | | |
| practice/regulations) | | | | | | | |
| Coagulation panel | X | X | X | X | X | X | X |
| Chemistry (long panel) including | X | X | X | X | X | X | X |
| LDH¹³ | | | | | | | |
| Hematology¹⁴ | X | X | X | X | X | X | X |
| Pregnancy test (WOCBP only)¹⁵ | | X | X | X | X | X | X |
| Urinalysis | X | X | X | X | X | X | X |

Pharmacokinetics, total C5, and Immunogenicity:

| | | | |
|---|---|---|---|
| Blood samples for conc. of | X | X | X |
| Pozelimab¹⁶ | | | |
| Blood samples for conc. of | X | X | X |
| Cemdisiran and metabolites¹⁷ | | | |
| Blood samples for conc. of total C5¹⁶ | X | X | X |
| Blood samples for immunogenicity of | X | X | X |
| Pozelimab¹⁸ | | | |
| Blood samples for immunogenicity of | X | X | X |
| Cemdisiran¹⁸ | | | |

Biomarkers:

| | | | | | |
|---|---|---|---|---|---|
| Free hemoglobin | X | | | | X |
| Haptoglobin | X | | | | X |
| Complement hemolytic assay (serum | X | X | | X | X |
| CH50)19 | | | | | |
| Complement hemolytic assay (serum | X | X | | X | X |
| AH50)19 | | | | | |
| sC5b-9 (plasma) | X | X | | X | X |
| PNH erythrocyte cells | | | X | | X |
| PNH granulocyte cells | X | | X | | X |

Optional research:

| | | |
|---|---|---|
| Future research serum and plasma | X | X |
| (optional) | | |
| Whole blood RNA sample (optional) | X | X |

Footnotes
¹Visits may be at the clinical site or another preferred location, such as the patient's home. The location will depend on the availability of a home healthcare visiting professional as well as the preferences of the investigator and patient. In the event of travel restrictions due to a global pandemic, alternative mechanisms such as but not limited to telemedicine visits may be implemented to maintain continuity of study conduct.
²Study procedures (visits): when multiple procedures are performed on the same day, the sequence of procedures is as follows: COA assessments, ECG and/or vital signs, blood collection, study treatment administration, and any pre-specified post-dose sample collection.
³Day 1e of OLEP should be scheduled on the same day as week 32 (or week 32r for patients on intensified treatment) of the OLTP, and any common assessments will be performed once for both the OLTP and OLEP visits.
⁴During the OLEP, the dose of Cemdisiran and Pozelimab SC should be given on the day of the corresponding study visit whenever possible. Study treatment administration should always be the last procedure after all blood sample collection and study assessments have been completed unless otherwise specified. If Pozelimab or Cemdisiran cannot be administered on the day of the corresponding study visit, the combination may be administered up to 3 days before or up to 3 days after the planned dosing date for patients in an intensified treatment regimen (Pozelimab Q2W dosing) or up to 7 days before or 7 days after the planned dosing date for patients on a maintenance treatment regimen (Pozelimab Q4W dosing), provided that the dosing takes place after the corresponding study visit has been completed. Care must be taken to coordinate dosing for visits where a post-dose sample is collected to measure concentration of Cemdisiran and its metabolites.
⁵For patients who did not receive intensified treatment during OLTP: at any time during the OLEP, patients who meet pre-specified criteria will receive intensified treatment consisting of a Pozelimab 30 mg/kg IV loading dose followed 30 minutes later by the initiation of Pozelimab 400 mg SC Q2W and Cemdisiran 200 mg SC Q4W. Patients will continue their visit schedule at the next OLEP visit.
⁶Study treatment administration should always be the last procedure after all blood sample collection and study assessments have been completed unless otherwise specified. For patients whose treatment is not intensified during the OLEP, the last doses of Cemdisiran and Pozelimab are administered at week 52e. For patients whose treatment was intensified during the OLEP, the last doses of study treatment will be determined based on the time of treatment intensification.
⁷Injection training will be provided to patients who desire self-injection or injection by a designated person. The site should observe patient self-injection or injection by a designated person and confirm adequacy. Patient instruction materials will be provided.
⁸If study treatment is given by the patient or by a designated person, the patient will complete a diary for recording compliance with study treatment administration. If the patient diary is provided to the patient, then it should be reviewed at each clinic visit and data collected into the CRFs. On the final visit, the diary should be collected by the site.
⁹Daily oral antibiotic prophylaxis against N. meningitidis is recommended until 52 weeks after discontinuation of study treatment.
¹⁰Patient safety card: the site should review the instructions on the safety card with the patient at each visit.
¹¹Breakthrough hemolysis assessment: if a patient is suspected of having a breakthrough hemolysis event, then in addition to the required laboratory collection, additional samples for coagulation parameters, chemistry, hematology, reticulocyte count, D-dimer, total C5, CH50, ADA (against Pozelimab), and drug concentrations of Pozelimab will be collected unless already noted in the Schedule of Events for that visit. If the suspected event does not occur at a scheduled visit, then an unscheduled visit should occur with an evaluation of the patient and collection of coagulation parameters, chemistry, hematology, reticulocyte count, total C5, CH50 and drug concentrations of Pozelimab.
¹²Clinical lab samples will be collected prior to any study drug administration (pre-dose) unless otherwise specified. The coagulation blood sample (tube) must always be collected first, followed by the blood chemistry sample (tube). During lab collection, handling, and processing, the same methodology will be applied across study visits, as best as possible, to preserve the quality of the sample and avoid hemolysis.
¹³Serum LDH, CRP, and bilirubin will be assessed as part of the blood chemistry analysis. The blood chemistry sample should be collected before study treatment administration (pre-dose). If the investigator or sponsor suspects that the lab result is not an accurate reflection of the patient's condition, the lab sample should be repeated.
¹⁴The hematology sample should be collected before study treatment administration (pre-dose).
¹⁵Pregnancy test for WOCBP: a urine test will be done at all visits indicated. Any positive urine pregnancy test should be confirmed with a serum pregnancy test.
¹⁶Blood samples for Pozelimab concentration analysis and total C5 analysis will be obtained on the specified days prior to any study treatment administration (pre-dose). If the patient receives treatment intensification during the open-label extension period, a PK sample should be obtained prior to IV Pozelimab administration and 15 minutes post-dose.
¹⁷Blood samples for Cemdisiran concentration analysis and concentrations of its metabolites will be collected on the specified days prior to any study treatment administration (pre-dose) and 2 to 6 hours post-Cemdisiran administration. The post-dose sample should be carefully coordinated with the dosing of Cemdisiran and may be collectedat the clinic or by a visiting healthcare professional.
¹⁸Blood samples for immunogenicity will be collected on the specified days prior to any study treatment administration (pre-dose). At the visits where immunogenicity samples are to be taken, the sample should be collected with the drug concentration sample. In the event of suspected treatment-related SAEs, such as anaphylaxis or hypersensitivity, additional drug concentration and immunogenicity samples may be collected at or near the onset and the resolution of the event.
19Blood samples for CH50 (efficacy endpoint) and AH50 will be obtained prior to any study treatment administration (pre-dose).

TABLE 5-5
Schedule of Events for Post-Treatment Safety Follow-Up Period (All Patients)

| | Study Procedure | | | | | |
|---|---|---|---|---|---|---|
| | 52-Week Post-Treatment Safety Follow-Up Period | | | | | |
| | | | | | Phone | Phone |
| | | | | | visit | visit |
| Visit # ¹ | FU-1 | FU-2 | FU-3 | FU-4 | FU-5 | FU-6 |
| Week (after last dose of study drug) | 8 | 12 | 16 | 26 | 38 | 52 |
| Day | 253 | 281 | 309 | 379 | 463 | 561 |
| Window (day) | ±10 | ±10 | ±10 | ±10 | ±10 | ±10 |

Safety Assessments:

| Patient safety card for | <------------------------------------------------X---------------------------------------> | | | | | |
|---|---|---|---|---|---|---|
| Neisseria meningitidis2 | | | | | | |
| Antibiotics prophylaxis | <---------------------------------------------X------------------------------------------> | | | | | |
| (recommended)3 | | | | | | |
| Vital signs | X | X | X | X | | |
| Physical examination | | X | | X | | |
| Concomitant meds and | X | X | X | X | X | X |
| procedures | | | | | | |

| | |
|---|---|
| Adverse events | <------------------------------------------X------------------------------------------> |
| Pregnancy reporting | <------------------------------------------X------------------------------------------> |

Laboratory Testing:

| | | | | |
|---|---|---|---|---|
| Hematology | X | X | X | X |
| Chemistry | X | X | X | X |

Footnotes
¹ Patients who discontinue study treatment in either the OLTP or the intensified OLTP will be asked to remain in the study until week 32 EOT (or week 32r EOT) and follow the original Schedule of Events as applicable. After the week 32 or week 32r EOT visit, the entry point into the safety follow-up schedule will depend on the number of weeks that have elapsed since patient's last dose (e.g., a patient who is 20 weeks after his/her final dose of study treatment at EOT will enter into the safety follow-up period at Visit FU-4 [26 weeks after last dose]). Patients who completed week 32r in the OLTP who choose not to continue treatment in the OLEP, patients who complete the optional OLEP but do not continue study treatment in a post-trial access program, and patients who permanently discontinue treatment during the OLEP will enter into the safety follow-up period at FU-1.
2Patient safety card: Site should review the instructions on the safety card with the patient at each visit. Replacement cards may be given to the patient as needed.
3Daily oral antibiotic prophylaxis against N. meningitidis is recommended until 52 weeks after discontinuation of study treatment.

### Primary Objective

The primary objective of the study is to evaluate the safety and tolerability of Pozelimab and Cemdisiran combination therapy in patients with PNH who switch from Eculizumab therapy.

### Secondary Objectives

The secondary objectives of the study are:
To evaluate the effect of the combination treatment on the following parameters of intravascular hemolysis: LDH control, breakthrough hemolysis, and inhibition of CH50
To evaluate the effect of the combination treatment on the stability of LDH during the transition period from Eculizumab monotherapy to combination with Pozelimab and Cemdisiran
To evaluate the effect of the combination treatment on red blood cell (RBC) transfusion requirements
To evaluate the effect of the combination treatment on hemoglobin levels
To evaluate the effect of the combination treatment on clinical outcome assessments (COAs) measuring fatigue and health related quality of life (HRQoL)
To assess the concentrations of total Pozelimab and Eculizumab in serum, and total Cemdisiran and C5 protein in plasma
To assess the immunogenicity of Pozelimab and Cemdisiran
To assess safety after dose intensification
To evaluate the long-term safety and efficacy of the combination treatment in an optional open-label extension period (OLEP)

### Exploratory Objectives

The exploratory objectives of the study are:
To explore the need for intensified treatment;
To explore the effect on clinical thrombosis events;
To explore the effect on renal function and renal injury biomarkers;
To explore the effect on complement activation and intravascular hemolysis relevant to PNH and other related diseases;
To explore the effect on PNH clone size;
To evaluate the effect on a COA measuring treatment satisfaction (TSQM);
To explore the effect on a novel COA measuring PNH-specific symptoms;
To explore the effect on PNH symptoms;
To explore potential differences in genotype and gene expression that may influence efficacy and safety of the combination treatment for further understanding of C5, PNH, or other conditions associated with complement-mediated injury (for patients who consent to participate in a genomics sub-study);
To explore Pozelimab and Cemdisiran mechanism of action (related to efficacy and/or safety), complement pathway biology, PNH, and related complement-mediated diseases;
To explore efficacy after dose intensification with Pozelimab and Cemdisiran;
To explore the long-term effects of the combination treatment on clinical and PD assessments in an optional OLEP.

### Primary Endpoint

The primary study endpoint is the incidence and severity of TEAEs through day 225 of the OLTP.

### Secondary Endpoints

The secondary endpoints for the OLTP are:
The percent change in LDH from pre-treatment (as defined by the mean of the LDH values at the screening visit [obtained no more than one day before administration with Eculizumab] and baseline (day 1 visit, prior to administration of Cemdisiran and Eculizumab) to end-of-treatment period (as defined by the mean of the LDH values at days 197 and 225 in the OLTP);
The percent change in LDH from pre-treatment to day 29;
The proportion of patients who are transfusion-free (defined as not requiring an RBC transfusion as per protocol algorithm, i.e., transfusion avoidance) from baseline through day 225, and from day 29 through day 225, inclusive;
The rate and number of units of RBCs transfused from baseline through day 225, and from day 29 through day 225, inclusive. The proportion of patients with breakthrough hemolysis from baseline through day 225, and from day 29 through day 225, inclusive
The proportion of patients who maintain adequate control of hemolysis, defined as LDH≤1.5×ULN from post-baseline (on day 1) through day 225, and from day 57 through day 225, inclusive;
The proportion of patients with adequate control of hemolysis at each visit from postbaseline (on day 1) through day 225, inclusive;
The proportion of patients with normalization of their LDH at each visit, defined as LDH≤1.0×ULN, from post-baseline (on day 1) through day 225, inclusive;
The area under the curve (AUC) of LDH over time between baseline through day 225, and from day 57 through day 225, inclusive;
The proportion of patients with hemoglobin stabilization (defined as patients who do not receive an RBC transfusion and have no decrease in hemoglobin level of ≥2 g/dL) from baseline through day 225, and from day 29 through day 225, inclusive;
The change in hemoglobin levels from baseline to day 225, inclusive;
Change in fatigue as measured by the FACIT-Fatigue Scale from baseline to day 225, inclusive;
Change from baseline to day 225 in global health status/QoL scale (GHS) and physical function (PF) scores on the European Organization for Research and Treatment of Cancer: Quality-of-Life Questionnaire core-30 items (EORTC QLQ-C30);
Change in total CH50 from baseline to day 225, inclusive;
Concentrations of total Pozelimab and Eculizumab in serum, and total Cemdisiran in plasma assessed throughout the study;
Change from baseline in concentration of total C5 assessed throughout the study;
Assessment of immunogenicity to Pozelimab and Cemdisiran as determined by the incidence, titer, and clinical impact of treatment-emergent anti-drug antibody (ADA) responses over time;
Incidence and severity of TEAEs for patients who receive dose intensification through day 225r;

The secondary endpoints for the optional OLEP are:
Incidence and severity of TEAEs during the 52-week OLEP in patients treated with Pozelimab and Cemdisiran combination therapy;
Change and percent change of LDH from day 1e (baseline of the OLEP) to week 24e and week 52e;
The proportion of patients who are transfusion-free (defined as not requiring an RBC transfusion as per protocol algorithm) from day 1e through week 24e and week 52e (i.e., transfusion avoidance);
The rate and number of units of RBCs transfused from day 1e through week 24e and week 52e;
The proportion of patients with breakthrough hemolysis from day 1e through week 24e and week 52e;
The proportion of patients who maintain adequate control of their hemolysis, defined as LDH≤1.5×ULN between day 1e through week 24e and week 52e, inclusive;
The proportion of patients with adequate control of hemolysis at each visit from day 1e through week 24e and week 52e, inclusive;
The proportion of patients with normalization of their LDH, defined as LDH≤1.0×ULN at each visit from day 1e through week 24e and week 52e, inclusive;
The AUC of LDH over time between day 1e through week 24e and week 52e, inclusive • The proportion of patients with hemoglobin stabilization (defined as patients who do not receive an RBC transfusion and have no decrease in hemoglobin level of ≥2 g/dL) from day 1e through week 24e and week 52e;
The change in hemoglobin levels from day 1e to week 24e and week 52e of the OLEP;
Change in fatigue as measured by the FACIT-Fatigue Scale from day 1e to week 52e of the OLEP;
Change from day 1e to week 52e of the OLEP in GHS/QoL scale PF scores on the EORTC QLQ-C30;
Change in CH50 from day 1e to week 16e, week 24e, and week 52e of the OLEP;
Concentrations of total Pozelimab in serum, and total C5 and Cemdisiran in plasma, assessed over time during the OLEP;
Assessment of immunogenicity to Pozelimab and Cemdisiran as determined by the incidence, titer, and clinical impact of treatment-emergent ADA responses over time during the OLEP.

### Exploratory Endpoints

The exploratory endpoints for the OLTP are:
Proportion of patients who require treatment intensification throughout the study;
Incidence of MAVE, defined as adverse events of special interest that include thrombophlebitis/deep vein thrombosis, pulmonary embolus, myocardial infarction, unstable angina, renal vein or artery thrombosis, acute peripheral vascular occlusion, hepatic vein thrombosis, portal vein thrombosis, mesenteric/visceral vein thrombosis or infarction, mesenteric/visceral arterial thrombosis or infarction, transient ischemic attack, cerebral arterial occlusion/cerebrovascular accident, cerebral venous occlusion, gangrene (nontraumatic; non-diabetic), amputation (nontraumatic; non-diabetic) from baseline through day 225;
Change in renal function as measured by estimated glomerular filtration rate (eGFR) from baseline to day 225;
Percent change in free hemoglobin from baseline to day 225;
Change in bilirubin from baseline to day 225;
Change in reticulocyte count from baseline to day 225;
Change and percent change in AH50 from baseline to day 225;
Proportion of PNH erythrocytes and granulocytes from baseline to day 225;
Change from baseline to day 225 in functional scale scores (Role Functioning, Emotional Functioning, Cognitive Functioning, and Social Functioning) and symptom scale scores (Fatigue, Nausea and vomiting, Pain, Dyspnoea, Insomnia, Appetite Loss, Constipation, Diarrhoea) of the EORTC QLQ-C30;
Proportion of patients with stability in global health status, functioning, and symptoms as measured by the EORTC QLQ-C30 from baseline to day 225;
Comparison of treatment satisfaction (as assessed by the TSQM) at baseline (with Eculizumab) versus treatment at day 225 (with Pozelimab/Cemdisiran);
Change in PNH symptoms as measured by the de novo PNH Symptom-Specific Questionnaire from baseline to day 225;
Change in Patient Global Impression of Severity (PGIS) from baseline to day 225, including questions on PNH symptoms, impacts, and fatigue;
Patient Global Impression of Change (PGIC) at day 225, including questions on PNH symptoms, impacts, and fatigue;
Identify any potential differences that influence efficacy and safety via genotyping and gene expression analysis (by RNA sequencing).

### Efficacy Variables

Laboratory Variables for the Assessment of Efficacy-Efficacy in this study is evaluated by the following laboratory assessments:
LDH (serum)
Hemoglobin
CH50

These laboratory variables are relevant to the characterization and disease mechanisms of PNH (Brodsky, Paroxysmal nocturnal hemoglobinuria, Blood 2014; 124(18):2804-11). Lactate dehydrogenase as a measure of intravascular hemolysis allows for an objective and precise means to gauge whether the control of intravascular hemolysis with Eculizumab is sustained when the patients are switched to Pozelimab and Cemdisiran combination treatment. The CH50 assay will be used to confirm complete inhibition of complement activity has been achieved throughout the dosing interval in patients with PNH.

### Transfusion Record

Hemolytic anemia is a clinical manifestation of PNH, and patients often require blood transfusion for symptomatic management. The frequency of blood transfusion has been used in other studies of PNH to assess efficacy (Hillmen, 2006) (Röth, 2018).

### Clinical Outcome Assessments

The following clinical outcome assessments will be completed by the patient:
FACIT-Fatigue;
EORTC QLQ-C30;
TSQM;
PNH Symptom-Specific Questionnaire;
PGIS;
PGIC.

### Safety Variables:

The safety variables in this study include:
TEAEs;
Body weight;
Vital signs;
Electrocardiogram (ECG);
Physical examination;
Routine safety laboratory tests (hematology, chemistry, urinalysis, and pregnancy testing [for women of childbearing potential or WOCBP]);
Concomitant medications and treatments.

### Screening Period

The screening period will evaluate patients to establish their eligibility to enter the study.

The screening visit should take place up to 42 days prior to day 1 (a day that the patient is scheduled to be administered Eculizumab). An additional interim screening visit(s) may take place as needed, for instance, in order to obtain the LDH value for pretreatment assessment on the day of (or if not possible, one day before) Eculizumab administration, and prior to Eculizumab dosing.

Historical data will be collected including, but not limited to, Eculizumab administration, concomitant medications, hemolytic parameters, and transfusions. Data will also be collected on PNH signs and symptoms during the screening period.

Patients will require vaccination/revaccination for N. meningitidis unless documentation is provided of prior immunization in the past 5 years prior to screening, or less than 5 years if required according to current national vaccination guidelines for vaccination use with complement inhibitors or local practice. For patients who require meningococcal vaccination during the screening period, administration should occur preferably at least 2 weeks prior to day 1, unless local practice or national guidelines specify a different vaccination protocol. If vaccination precedes the initiation of study treatment by less than 2 weeks, then the patient must receive antibiotic prophylaxis for a minimum of 2 weeks from the date of vaccine administration

During screening, patients who have not been vaccinated against Streptococcus pneumoniae and Haemophilus influenza type B according to current national/local vaccination guidelines will be required to be vaccinated to be eligible for the study.

In addition to N. meningitidis, fatal or serious infections with N. gonorrhea have been reported in patients taking Eculizumab. Therefore, a risk assessment and counseling regarding the potential risk of N. gonorrhea infection will be conducted per local practice.

Patients will be assessed for active or latent tuberculosis infection based on local practice or applicable guidelines. Based on the risk assessment, the need for screening with either tuberculin skin test or T-cell interferon-gamma release assay will be made. The interpretation of these results, as applicable, will be made by the investigator. Further management and treatment will be the responsibility of the investigator.

In addition to screening procedures, patients will be asked to complete a PNH Symptom-Specific Questionnaire daily for at least 14 consecutive days prior to the day 1 visit.

Patients may choose to participate in the optional OLEP, optional future biomedical research, and/or optional pharmacogenomics component of the study by signing the respective optional informed consent forms (ICFs).

### Open-Label Treatment Period

Patients who fulfill all the eligibility criteria will be enrolled in the study and receive their first dose of study drug on day 1, which should occur on the day of the patient's scheduled treatment with Eculizumab. As described herein, patients will be administered Cemdisiran with Eculizumab on day 1 (Note: Eculizumab may be administered up to 2 days after day 1 Cemdisiran in order to accommodate the logistical complexities associated with its administration). Eculizumab will be administered alone on day 15 (+2 days or earlier, according to the patient's usual dosing frequency. The first dose of combination SC therapy with Pozelimab and Cemdisiran without background Eculizumab will be administered on day 29 and continue Q4W thereafter. The first dose of combination SC therapy on day 29 will be preceded by an IV loading dose of Pozelimab to achieve high concentrations of Pozelimab rapidly in order to provide complete inhibition of C5, as soon as possible, during the switch from Eculizumab to Pozelimab. The SC doses should not be given until at least 30 minutes after completion of the IV administration, and the patient should be observed during the interval. Patients will also be monitored for at least 30 minutes after completing the first SC injections for the Pozelimab and Cemdisiran combination.

After the first SC dose of the Pozelimab and Cemdisiran combination on day 29, subsequent study treatment administrations may be continued by the site personnel, a healthcare professional if available, or administered by the patient or designated person at the patient's preferred location. These options for study treatment administration will depend on preference of the investigator and patient, local regulations, and availability of healthcare professional. If self-administration (or administration by a designated person) is undertaken, then sufficient injection training at the scheduled administration(s) with a Pozelimab and Cemdisiran maintenance regimen will be provided by the investigator or qualified study staff designee. After training, observation of self-administration (or administration by designated person) will be conducted by clinical site personnel and may be conducted in person at the patient's home or via telemedicine. Once this observation is considered satisfactory, the Pozelimab and Cemdisiran maintenance regimen can be subsequently administered independently by patient/designated person for the remainder of the study. A patient diary will be provided prior to initiation of self-administration for recording data on study treatment administration. The diary should be completed upon each study drug administration.

### Safety Considerations

Breakthrough hemolysis is assessed by the investigator throughout the study and is as defined herein. During the study, a patient meeting criteria for breakthrough hemolysis or inadequate LDH response may qualify for treatment intensification as described herein.

### Breakthrough Hemolysis

Breakthrough hemolysis is defined as an increase in LDH with concomitant signs or symptoms associated with hemolysis:
An increase in LDH occurs when:
   LDH≥2×ULN if pre-treatment LDH is <1.5×ULN; or
   LDH≥2×ULN subsequent to initial achievement of LDH≤1.5×ULN if pre-treatment LDH is >1.5×ULN. Note: pre-treatment LDH is defined by the mean of the LDH values at the screening visit and day 1 visit
The signs or symptoms should correspond to those known to be associated with intravascular hemolysis due to PNH, limited to the following: new onset or worsening fatigue, headache, dyspnea, hemoglobinuria, abdominal pain, scleral icterus, erectile dysfunction, chest pain, confusion, dysphagia, new thrombotic event, anemia including hemoglobin value significantly lower (i.e., ≥2 g/dL decrease) as compared to patient's known baseline hemoglobin values.

The decision to transfuse with RBCs during the study should proceed according to the criteria herein.

Transfusions with RBCs during the screening period and while the patient is receiving study treatment may proceed according to the following predefined criteria that will trigger a transfusion as clinically indicated, however the actual number of units to be transfused is at the discretion of the investigator:
Transfuse with RBC(s) if the hemoglobin level is 59 g/dL with new onset or worsening signs or symptoms resulting from anemia that are of sufficient severity to warrant transfusion; or
Transfuse with RBC(s) if the hemoglobin level is 57 g/dL with or without signs or symptoms of anemia.

Patients should be closely monitored for the entire study for early signs and symptoms of meningococcal infection and evaluated immediately if an infection is suspected. Patients will be provided a patient safety card describing signs and symptoms of suspected meningococcal infection along with instructions to follow in case of a potential meningococcal infection as well as information for the non-investigator healthcare provider for awareness. Daily oral antibiotic prophylaxis is recommended.

During intravenous infusion of Pozelimab, due to concern of potential IV infusion reactions, patients should be observed for at least 30 minutes after the infusion. In addition, emergency equipment and medication for the treatment of infusion reactions must be available at the clinical site for immediate use. All infusion reactions must be reported as adverse events (AEs) and graded. Patients should also be observed for at least 30 minutes after completing the first SC injections of the Pozelimab and Cemdisiran combination.

During the transition of therapy from Eculizumab to Pozelimab, investigators should have heightened awareness for possible AEs as a result of the risk of formation of large multimers of complexes of Eculizumab-C5-Pozelimab (i.e., large drug-target-drug (DTD) immune complexes).

### Study Procedures

Study procedures in the treatment period include laboratory assessments of efficacy (LDH, hemoglobin, and CH50), transfusion record update, clinical outcome assessments, body weight, and routine safety assessments (vital signs, physical examination, ECG, safety laboratory testing). Treatment-emergent adverse events and concomitant medications will be monitored throughout the study. Patients will provide blood samples for biomarkers, drug concentration for potential PK and PD assessment, immunogenicity, and exploratory assessments. Study procedures are listed by visit in Table 5-2 and described herein. Study procedures, including sample collection for laboratory analysis, may be performed at the study clinic or at another location that is more convenient for the patient (including home visits), if this option is available and with approval. The last doses of study treatment for patients who do not receive dose intensification are administered on day 197 (week 28). Patients will return for safety, efficacy, and other assessments at the end of treatment (EOT) visit at week 32. For patients who restarted on intensified treatment during the study, the last dose of study treatment is administered at week 30. Patients will return for safety, efficacy, and other assessments at the EOT visit at week 32 (Table 5-3).

### Optional Open-Label Extension Period

All patients who complete the OLTP study treatment, including patients who received the intensified regimen, will be offered the opportunity to continue in an optional 52-week OLEP, whereby the treatment with Pozelimab and Cemdisiran from the OLTP to the OLEP is planned to be uninterrupted (i.e., day 1e visit of the OLEP will correspond to the EOT visit in the OLTP, and any common assessments will be performed once for both visits). Study assessments and conduct for the optional OLEP are as described previously for the OLTP and are detailed in Table 5-4 (Schedule of Events for the optional OLEP).

During the optional OLEP, patients who are not on intensified treatment who meet criteria herein for treatment intensification will follow the dosing regimen as described herein with the new regimen starting on the day of intensification, and will continue their visit schedule at the next OLEP visit.

This means that starting the day of intensification (day pre-defined criteria have been met), the patient will receive a single administration of Pozelimab 30 mg/kg IV followed by SC administration of Pozelimab and Cemdisiran the same day. Thereafter, the patients will receive Pozelimab 400 mg Q2W and Cemdisiran 200 mg Q4W starting from the day of intensification (±3 days). The visit schedule for the OLEP will remain unchanged.

For patients who complete the optional OLEP, post-trial access to treatment may be available.

### Treatment Intensification

Patients who meet criteria for treatment intensification will receive a single administration of Pozelimab 30 mg/kg IV on the day of initiation (can be initiated from day 57 onward) in addition to a maintenance regimen with a shortened frequency of Pozelimab administration 400 mg SC Q2W along with Cemdisiran 200 mg SC Q4W (±3 days) for a period of 32 weeks starting on the day of initiation.

An intensified regimen is available to provide extra C5 suppression that some patients may need and which cannot be managed adequately by the standard dose regimen. Patients will receive intensification of their Pozelimab treatment from day 57 onward as described herein if they meet both criteria below:
Breakthrough hemolysis that is not due to a complement-activating condition (i.e., intercurrent infection); and
Inadequate LDH response (i.e., LDH>1.5×ULN) that is sustained (i.e., on 2 consecutive measurements spanning at least 2 weeks).

During the OLTP, patients who undergo treatment intensification may require unscheduled visit(s) before initiation. The day of initiation of the intensified regimen should be re-anchored to baseline of the Intensified Treatment Period in the OLTP (day 1r) and thereafter following a similar schedule of subsequent visits and assessments as newly enrolled patients (see Table 5-3). Patients who are on intensified treatment will be considered to have completed the OLTP once they finish the 32-week treatment period on the intensified regimen.

During the optional OLEP, patients who are not on intensified treatment who meet criteria for treatment intensification will follow the dosing regimen described herein with the new regimen starting on the day of intensification and for the remainder of the OLEP. Patients will continue their visit schedule at the next OLEP visit (Table 5-4).

Patients are eligible to receive intensification of Pozelimab only once (whether during the main treatment period or the optional OLEP), beyond which no further intensification will be permitted. Note: After day 29, patients who have an LDH increase ≥2×ULN due to an acute complement activating condition during the OLTP, intensified OLTP or optional OLEP may receive an IV Pozelimab dose of 30 mg/kg IV at the discretion of the investigator. An assessment of the patient's weight should be performed on the day of the IV Pozelimab load in order to calculate the appropriate dose. This is not considered treatment intensification as there will be no change in regimen and no requirement to reset their schedule to day 1.

An assessment of the patient's weight should be performed on the day of the IV Pozelimab load in order to calculate the appropriate dose. The IV dose should be administered first. The SC doses should be given at least 30 minutes after completion of the IV administration. Note: Patients who experience breakthrough hemolysis that is not due to a complement-activating condition, and meet criteria for treatment intensification, are eligible to receive intensification of Pozelimab only once (whether during the main treatment period or the optional OLEP), beyond which no further intensification will be permitted.

In the event of an LDH increase ≥2×ULN due to an acute complement-activating condition (i.e., intercurrent infection) after day 29 in the OLTP, intensified OLTP or at any time in the optional OLEP, an IV loading dose of Pozelimab 30 mg/kg IV may be given at the discretion of the investigator and in consultation with the sponsor. An assessment of the patient's weight should be performed on the day of the IV Pozelimab load in order to calculate the appropriate dose. This is not considered treatment intensification. No other changes will be made to the study treatment regimen (i.e., the regular dose and frequency of Pozelimab and Cemdisiran will proceed unchanged). Patients will continue onto the next visit of their current visit schedule.

### Results (5 Pts Completing Day 169)

At the time of this data analysis, 6 patients were enrolled, 5 of whom were ongoing. While one patient discontinued after 29 days of treatment, the remaining 5 patients had a treatment duration of at least 169 days, with one completing the OLTP (Day 225).

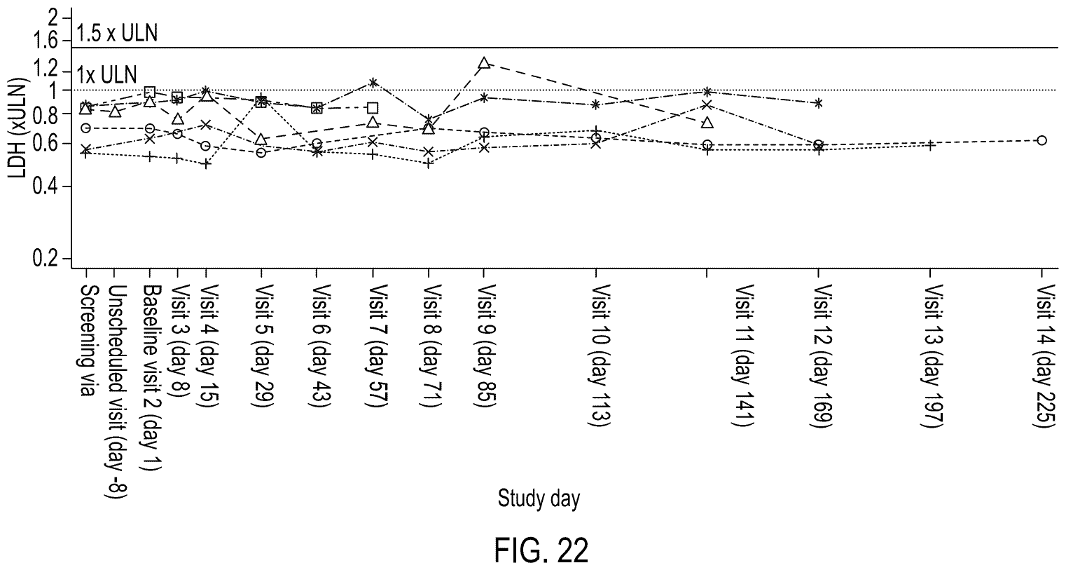

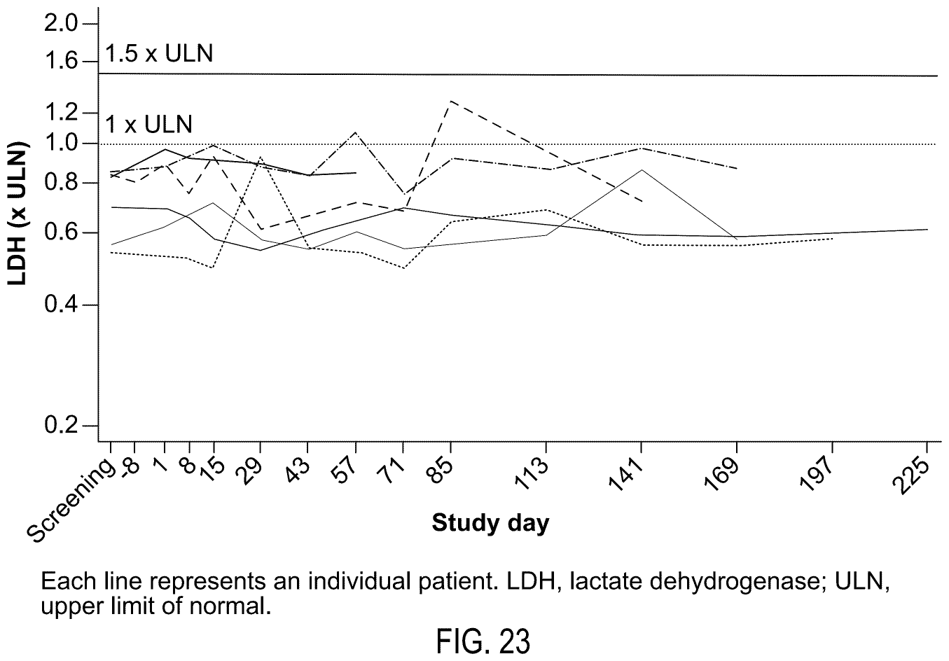

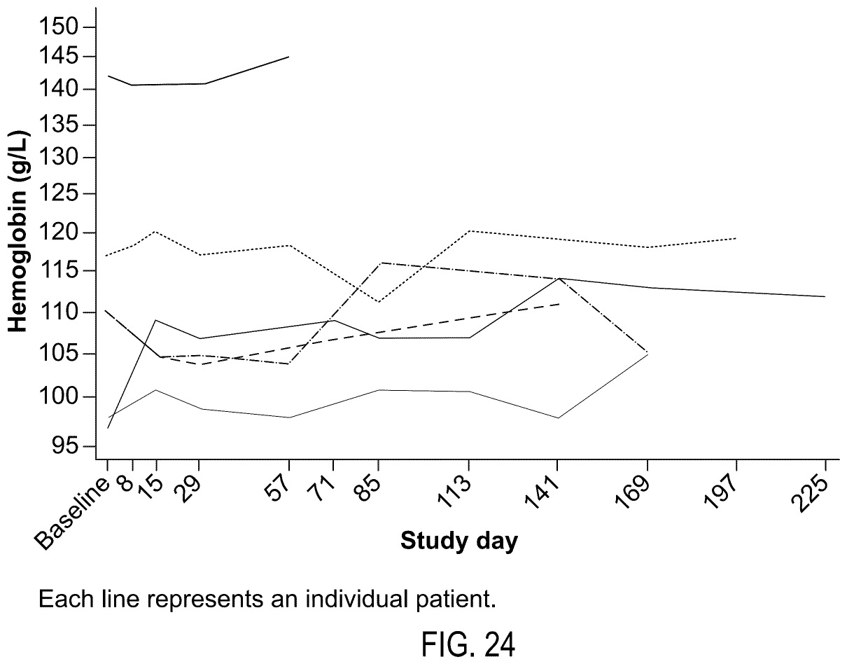

Up to the time of data cut-off, no patient had a lactate dehydrogenase (LDH) level greater than 1.5×ULN (FIG. 22 and FIG. 23) or experienced an event of breakthrough hemolysis, including the two patients who were previously treated with higher doses of Eculizumab (1200 mg and 1500 mg every 2 weeks, respectively). Furthermore, all but two LDH values remained normal (≤1.0×ULN) at all timepoints evaluated. All patients achieved hemoglobin stabilization (FIG. 24). No patient received a red blood cell transfusion nor had a decrease in hemoglobin ≥2 g/dl. CH50, a measure of terminal complement activity, remained fully suppressed at 0 klU/L throughout the study up to the time of the data cut-off.

There were no serious or severe TEAEs. Importantly, there were no meningococcal infections or adverse events due to potential large drug-target-drug immune complexes, or TEAEs leading to death, in this study. One patient discontinued study treatment due to two mild, non-serious TEAEs of headache at Days 2 and 16.

### Conclusions

These results show that, in patients with PNH transitioning from Eculizumab treatment, including patients treated with higher than standard of care doses, the combination of Pozelimab and Cemdisiran was generally well tolerated, providing sustained control of intravascular hemolysis without any breakthrough hemolysis events. Accordingly, this combination therapy demonstrates significant efficacy in treating patients with PNH who transitioned from Eculizumab therapy.

### Results (5 Pts Completing Day 225)

Six patients were enrolled. Five patients completed the OLTP (Day 225); one patient discontinued treatment after 29 days due to a treatment-emergent adverse event (TEAE) (Table 5-12).

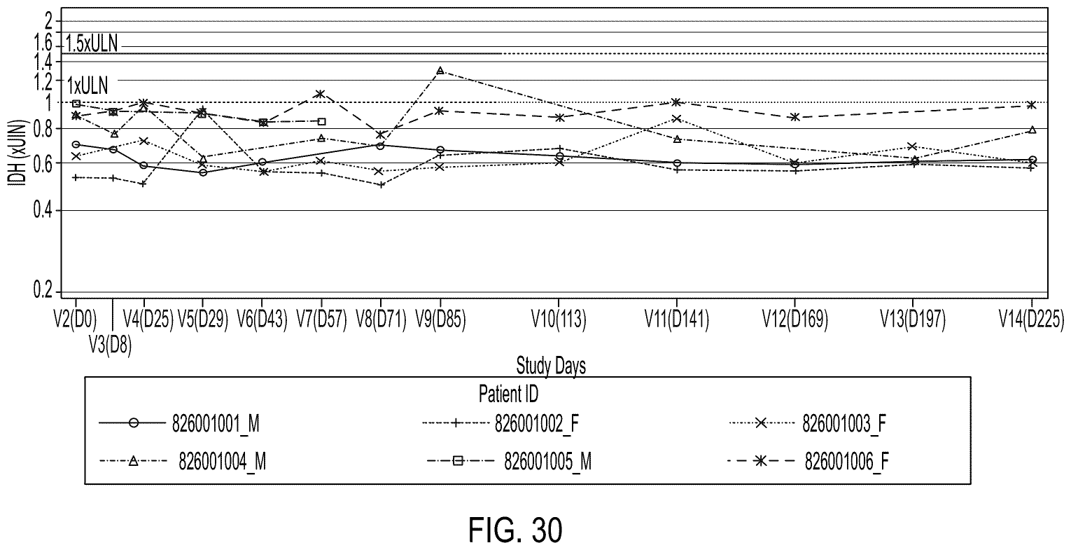

At baseline, lactate dehydrogenase (LDH) was well controlled on Eculizumab. In the year prior to enrollment, no patient had a history of a blood transfusion, but one patient had a history of breakthrough hemolysis. During the 32-week OLTP, no patient had an LDH greater than 1.5× the upper limit of normal (ULN; FIG. 30) or experienced an event of breakthrough hemolysis (Table 5-6), including the two patients who were previously treated with higher doses of Eculizumab (1200 mg and 1500 mg every 2 weeks, respectively). Furthermore, all but two LDH values remained normal (≤1.0×ULN) at all timepoints evaluated (FIG. 30). During the OLTP, all patients achieved hemoglobin stabilization (did not receive a red blood cell transfusion and had no decrease in hemoglobin level of ≥2 g/dL) (Table 5-7), and no patient required a blood transfusion. CH50, a measure of terminal complement activity, remained fully suppressed at 0 klU/L throughout the study (Table 5-8).

One patient discontinued study treatment due to two mild, non-serious TEAEs of headache at Days 2 and 16. One subject experienced a serious TEAE of endometrial hyperplasia for long standing, intermittent post-menopausal bleeding that required hospitalization for pre-emptive hysterectomy/ovariectomy; the event was deemed not related to the study treatment by the investigator and sponsor (Table 5-9). There were no meningococcal infections, TEAEs due to potential large drug-target-drug immune complexes, thrombotic events or TEAEs leading to death (Table 5-10 and Table 5-11).

### Summary/Conclusion

Results suggest that in patients with PNH transitioning from Eculizumab treatment (including patients receiving higher than standard doses), the combination of Pozelimab and Cemdisiran was generally well tolerated, providing sustained control of intravascular hemolysis without any breakthrough hemolysis events. Findings support the ongoing development of Pozelimab and Cemdisiran combination therapy.

TABLE 5-6
Breakthrough Hemolysis (Full Analysis Set)

| | Pozelimab 400 mg SC Q4W + |
|---|---|
| | Cemdisiran 200 mg SC Q4W |
| | (N = 6) |
| Patients (number and percentage) With Breakthrough | 0 |
| Hemolysis From Baseline to Day 225 | |
| Normal Approximation 95% Confidence Interval (%) | 0.0-0.0 |
| Clopper Pearson 95% Confidence Interval (%) | 0.0-45.9 |
| Patients (number and percentage) With Breakthrough | 0 |
| Hemolysis From Baseline to Day 29 to 225 | |
| Normal Approximation 95% Confidence Interval (%) | 0.0-0.0 |
| Clopper Pearson 95% Confidence Interval (%) | 0.0-45.9 |

TABLE 5-7
Hemoglobin Stabilization (Full Analysis Set)

| | Pozelimab 400 mg SC Q4W + |
|---|---|
| | Cemdisiran 200 mg SC Q4W |
| | (N = 6) |
| Patients (number and percentage) With Hemoglobin | 6 (100%) |
| Stabilization From Baseline to Day 225 | |
| Normal Approximation 95% Confidence Interval (%) | 100.0-100.0 |
| Clopper Pearson 95% Confidence Interval (%) | 54.1-100.0 |
| Patients (number and percentage) With Hemoglobin | 6 (100%) |
| Stabilization From Day 29 to Day 225 | |
| Normal Approximation 95% Confidence Interval (%) | 100.0-100.0 |
| Clopper Pearson 95% Confidence Interval (%) | 54.1-100.0 |

TABLE 5-8
Summary of CH50 (kIU/L)-Change from Baseline over Time (Full Analysis Set)

| | Pozelimab 400 mg SC Q4W + Cemdisiran 200 mg SC Q4W | | | | | | | | | |
|---|---|---|---|---|---|---|---|---|---|---|
| | (N = 6) | | | | | | | | | |
| | Value at Visit | | | | | Change from Baseline | | | | |
| Visit | n | Mean(SD) | Median | Q1:Q3 | Min: Max | n | Mean(SD) | Median | Q1:Q3 | Min: Max |
| BASELINE | 6 | 0.0(0.0) | 0.0 | 0:0 | 0:0 | | | | | |
| VISIT 3 (DAY 8) | 6 | 0.0(0.0) | 0.0 | 0:0 | 0:0 | 6 | 0.0(0.0) | 0.0 | 0:0 | 0:0 |
| VISIT 4 (DAY 15) | 6 | 0.0(0.0) | 0.0 | 0:0 | 0:0 | 6 | 0.0(0.0) | 0.0 | 0:0 | 0:0 |
| VISIT 5 (DAY 29) | 6 | 0.0(0.0) | 0.0 | 0:0 | 0:0 | 6 | 0.0(0.0) | 0.0 | 0:0 | 0:0 |
| VISIT 7 (DAY 57) | 6 | 0.0(0.0) | 0.0 | 0:0 | 0:0 | 6 | 0.0(0.0) | 0.0 | 0:0 | 0:0 |
| VISIT 9 (DAY 85) | 5 | 0.0(0.0) | 0.0 | 0:0 | 0:0 | 5 | 0.0(0.0) | 0.0 | 0:0 | 0:0 |
| VISIT 10 (DAY 113) | 4 | 0.0(0.0) | 0.0 | 0:0 | 0:0 | 4 | 0.0(0.0) | 0.0 | 0:0 | 0:0 |
| VISIT 11 (DAY 141) | 5 | 0.0(0.0) | 0.0 | 0:0 | 0:0 | 5 | 0.0(0.0) | 0.0 | 0:0 | 0:0 |
| VISIT 12 (DAY 169) | 5 | 0.0(0.0) | 0.0 | 0:0 | 0:0 | 5 | 0.0(0.0) | 0.0 | 0:0 | 0:0 |
| VISIT 13 (DAY 197) | 4 | 0.0(0.0) | 0.0 | 0:0 | 0:0 | 4 | 0.0(0.0) | 0.0 | 0:0 | 0:0 |
| | | | | | 0:0 | 5 | 0.0(0.0) | | | 0:0 |
| VISIT 14/EOT | 5 | 0.0(0.0) | 0.0 | 0:0 | | | | 0.0 | 0:0 | |
| (DAY 225)/OLEP-1 | | | | | | | | | | |
| (DAY 1e) | | | | | | | | | | |

TABLE 5-9
Summary of Treatment Emergent Serious Adverse Events by System
Organ Class and Preferred Term (Safety Analysis Set)

| System Organ | Pozelimab 400 mg SC Q4W + |
|---|---|
| Class MedDRA | Cemdisiran 200 mg SC Q4W |
| Preferred Term | (N = 6) |
| Number of Treatment-Emergent Serious Adverse Event | 1 |
| Number of subjects with at least one Treatment- | 1(16.7%) |
| Emergent Serious Adverse Event | |
| Reproductive system and breast disorders | 1(16.7%) |
| Endometrial hyperplasia | 1(16.7%) |

TABLE 5-10
Overview of Treatment Emergent Adverse Events
of Special Interest (Safety Analysis Set)

| AE of Special | Pozelimab 400 mg SC Q4W + |
|---|---|
| Interest Category | Cemdisiran 200 mg SC Q4W |
| Subcategory | (N = 6) |
| Number of TEAE of Special Interest | 23 |
| Number of subjects with at least one TEAE of Special | 5(83.3%) |
| Interest | |
| Suspected Neisseria infection | 0 |
| Moderate or severe infusion reactions | 0 |
| Any Thrombotic or embolic event | 0 |
| Moderate or severe hypersensitivity reactions potentially | 0 |
| related to study treatment | |
| Adverse events potentially due to suspected large DTD | 0 |
| immune complexes | |
| Liver transaminase elevations | 0 |
| Other AESIs (mild infusion reactions, mild hypersensitivity | 5(83.3%) |
| reactions or injection site reactions) | |
| Infusion reaction | 0 |
| Hypersensitivity or an allergic reaction | 0 |
| Injection site reaction | 5(83.3%) |

TABLE 5-11
Summary of Treatment Emergent Adverse Events Leading to Death
by System Organ Class and Preferred Term (Safety Analysis Set)

| System Organ | Pozelimab 400 mg SC Q4W + |
|---|---|
| Class MedDRA | Cemdisiran 200 mg SC Q4W |
| Preferred Term | (N = 6) |
| Number of TEAE Leading to Death | 0 |
| Number of subjects with at least on TEAE Leading to | 0 |
| Death | |

TABLE 5-12
Patient Disposition (Safety Analysis Set) Treatment group:
Pozelimab 400 mg SC Q4W + Cemdisiran 200 mg SC Q4W

| | | | | End of | |
|---|---|---|---|---|---|
| | | | End of Treatment | Treatment | |
| | | | Status of | Status of | End of Study |
| | | Last Dose | OLTP/Reason of trt. | OLEP/Reason | Status/Reason of |
| Subject ID | Age/Sex | (Study Day) | discont. | of trt. discont. | study discont. |
| 826001001 | 63/M | (253) | COMPLETED | ONGOING | ONGOING |
| 826001002 | 65/F | (253) | COMPLETED | ONGOING | ONGOING |
| 826001003 | 40/F | (225) | COMPLETED | ONGOING | ONGOING |
| 826001004 | 36/M | (225) | COMPLETED | ONGOING | ONGOING |
| 826001004 | 51/M | (29) | DISCONTINUED/ | | DISCONTINUED/ |
| | | | ADVERSE EVENT | | WITHDRAWAL |
| | | | | | BY SUBJECT |
| 826001006 | 53/F | (225) | COMPLETED | ONGOING | ONGOING |

### Results (5 Pts Completing Open Label Treatment Period: Median Treatment Duration 229 [Range: 56-280 Days])

Six patients were enrolled (Table 5-13), and five completed the OLTP (median [range] treatment duration: 229 [56-280] days). One patient discontinued study treatment after 29 days due to two mild, non-serious treatment-emergent adverse events (TEAEs) of headache at Days 2 and 16, both occurring prior to administration of the combination treatment at Day 29.

TABLE 5-13
Baseline Demographics and Clinical Characteristics

| | Pozelimab 400 mg Q4W + | |
|---|---|---|
| | cemdisiran 200 mg SC Q4W | |
| | (n = 6) | |
| Age, years, median (min:max) | 52.0 | (36:65) |
| Male sex, n (%) | 3 | (50.0) |
| Race, White, n (%) | 6 | (100.0) |
| BMI, kg/m², median (min:max) | 27.2 | (24.7:49.7) |
| Eculizumab dosage, n (%) | | |
| Standard (900 mg Q2W) | 4 | (66.7) |
| Non-standard (1200 or 1500 mg Q2W) | 2 | (33.3) |
| LDH,^{a} U/L, median (min:max) | 231.5 | (175:291) |
| Hemoglobin, g/L, median (min:max) | 110.0 | (97:142) |
| PNH RBCs total,^{b} % | | |
| Mean (SD) | 56.5 | (32.3) |
| PNH PMNs,^{b} % | | |
| Mean (SD) | 64.7 | (28.0) |
| PNH monocytes,^{b} % | | |
| Mean (SD) | 81.7 | (15.9) |
| Total bilirubin, μmol/L, median (min:max) | 12.2 | (8.7:19.5) |
| Reticulocytes, 10⁹/L, median (min:max) | 173.8 | (77.4:388.3) |
| eGFR, mL/min/1.73 m², | 109.5 | (80:121) |
| median (min:max) | | |
| Creatinine, μmol/L, median (min:max) | 57.5 | (35:88) |

^{a}Pre-treatment values.
^{b}Three samples were missing from the central laboratory, so local laboratory pre-study PNH clone data were used.
BMI, body mass index; eGFR, estimated glomerular filtration rate; LDH, lactate dehydrogenase; PMN, polymorphonuclear neutrophil; PNH, paroxysmal nocturnal
hemoglobinuria; Q2W, every 2 weeks; Q4W, every 4 weeks; RBC, red blood cell; SC, subcutaneous; SD, standard deviation.

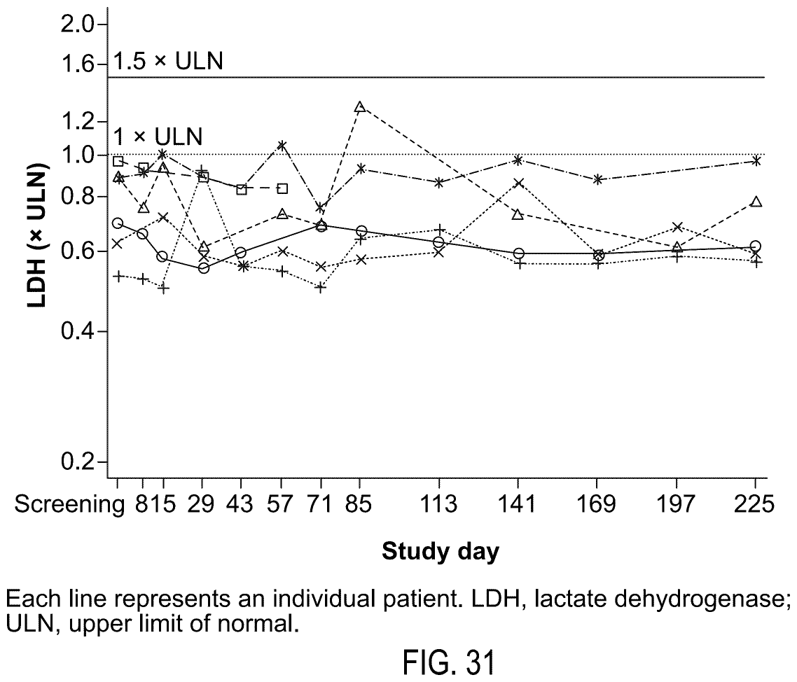

All patients had an LDH value of ≤1.5×ULN at all timepoints assessed; four out of the six patients maintained normal LDH values (≤1.0×ULN) at all time points evaluated (FIG. 31).

No patients experienced a breakthrough hemolysis event, including the two patients who were previously treated with higher doses of eculizumab (1200 mg or 1500 mg every 2 weeks, respectively).

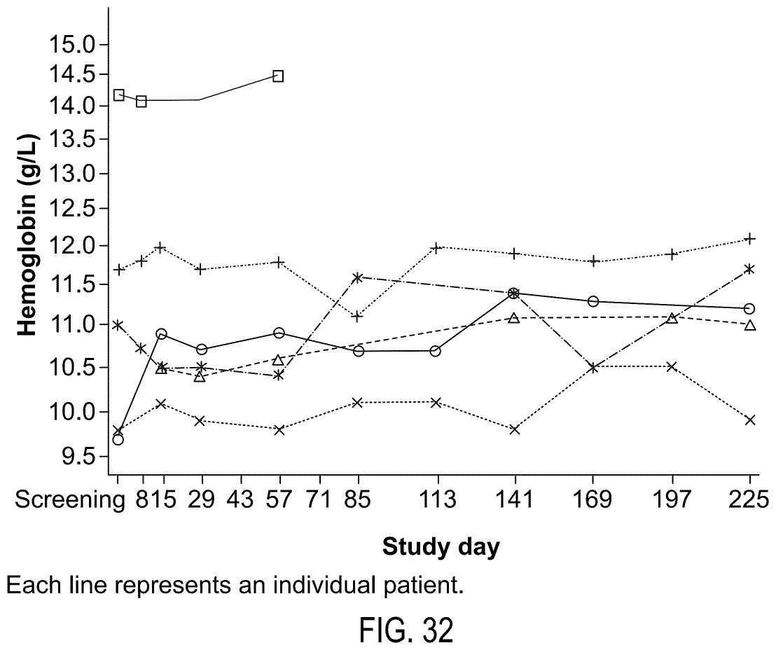

Individual patients' hemoglobin levels over time are shown in FIG. 32; no patients received a red blood cell transfusion, nor had a decrease in hemoglobin ≥2 g/dL.

CH50, a measure of terminal complement activity, remained fully suppressed at 0 klU/L throughout the study.

One patient experienced a serious and severe TEAE of endometrial hyperplasia for long standing, intermittent post-menopausal bleeding that required hospitalization for pre-emptive hysterectomy/ovariectomy; the event was deemed not related to study treatment.

The most common TEAEs were non-serious injection-site reactions (n=5, 83.3%; none led to treatment discontinuation), and headache (n=3, 50.0%). Importantly, there were no meningococcal infections, adverse events due to potential large drug-target-drug immune complexes, or TEAEs leading to death.

In patients with PNH transitioning from Eculizumab treatment, the combination of Pozelimab and Cemdisiran treatment was generally well tolerated and provided sustained control of intravascular hemolysis.

No patient experienced an event of breakthrough hemolysis.

Findings support the ongoing development of Pozelimab and Cemdisiran combination therapy.

### Results (52 Week Open-Label Extension Data)

Patients who completed the 32-week open-label treatment period (OLTP) were offered to participate in an optional 52-week OLEP. Patients were adults with PNH who had switched from stable Eculizumab therapy to the combination (pozelimab 400 mg and cemdisiran 200 mg) SC every 4 weeks in the OLTP. The study enrolled two patients who were previously treated with higher doses of Eculizumab (1200 mg or 1500 mg every two weeks).

All five patients who completed the OLTP were enrolled in and completed the OLEP. After completing the OLEP, all patients transitioned to an expanded access program to continue the combination of Pozelimab and Cemdisiran. At baseline of the OLTP, lactate dehydrogenase (LDH) was well controlled on Eculizumab and remained controlled during the 32-week OLTP. During the 52-week OLEP, no patient had an LDH greater than 1.5× the upper limit of normal (ULN; Figure) at any of the scheduled study visits or met the protocol criteria for breakthrough hemolysis (either by central or local laboratory values; defined as an increase in LDH [LDH≥2×ULN if pre-treatment LDH≤1.5×ULN, or LDH≥2×ULN subsequent to initial achievement of LDH≤1.5×ULN if pre-treatment LDH>1.5×ULN] with concomitant signs or symptoms associated with hemolysis). The two patients who previously received higher doses of eculizumab also maintained control of LDH levels throughout the OLTP and OLEP. Four of five patients remained transfusion free, but one patient required a blood transfusion while hospitalized with an acute complement-activating condition. This patient experienced two serious and severe treatment-emergent adverse events of respiratory infection and consequently acute hemolysis that did not meet the trial criteria for breakthrough hemolysis but was reported as an adverse event based on clinical judgement. The investigator and sponsor assessed these events as not treatment related. CH50, a measure of terminal complement activity at fixed time points, remained suppressed throughout the study in all patients. No other serious or severe adverse events were reported. There were no meningococcal infections, thrombotic events, or TEAEs leading to death.

Results suggest that, in patients with PNH transitioning from Eculizumab treatment, the combination of Pozelimab and Cemdisiran was generally well tolerated and provided long term sustained control of intravascular hemolysis without any breakthrough hemolysis events. Findings support the ongoing development of Pozelimab and Cemdisiran combination therapy.

### Example 6: A Randomized, Open-Label Eculizumab and Ravulizumab Controlled, Non-Inferiority Study to Evaluate the Efficacy and Safety of Pozelimab and Cemdisiran Combination Therapy in Patients with Paroxysmal Nocturnal Hemoglobinuria Who are Currently Treated with Eculizumab or Ravulizumab (R3918-PNH-2022)

This study is a randomized, open-label, Eculizumab and Ravulizumab-controlled, non-inferiority study. The study was terminated early due to recruitment issues.

Patients treated with Eculizumab will be eligible if they are taking Eculizumab at the labeled posology of 900 mg IV Q 14 days for at least 12 weeks prior to screening visit.

Patients treated with Ravulizumab will be eligible if they are taking IV Ravulizumab at the labeled posology Q8W based on body weight (BW) as follows: 3000 mg for BW≥40 kg to <60 kg, 3300 mg for BW≥60 kg to <100, 3600 mg for BW 100 kg for at least 24 weeks prior to screening visit.

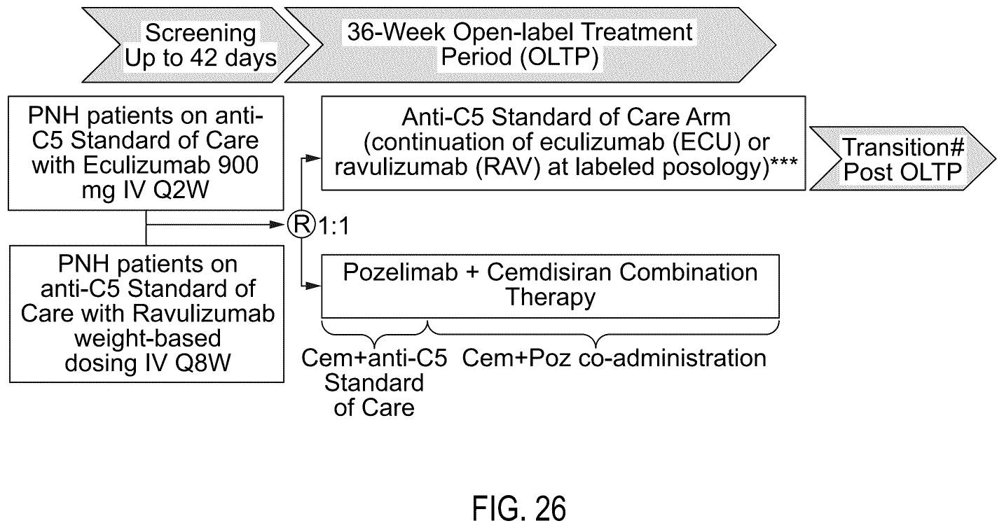

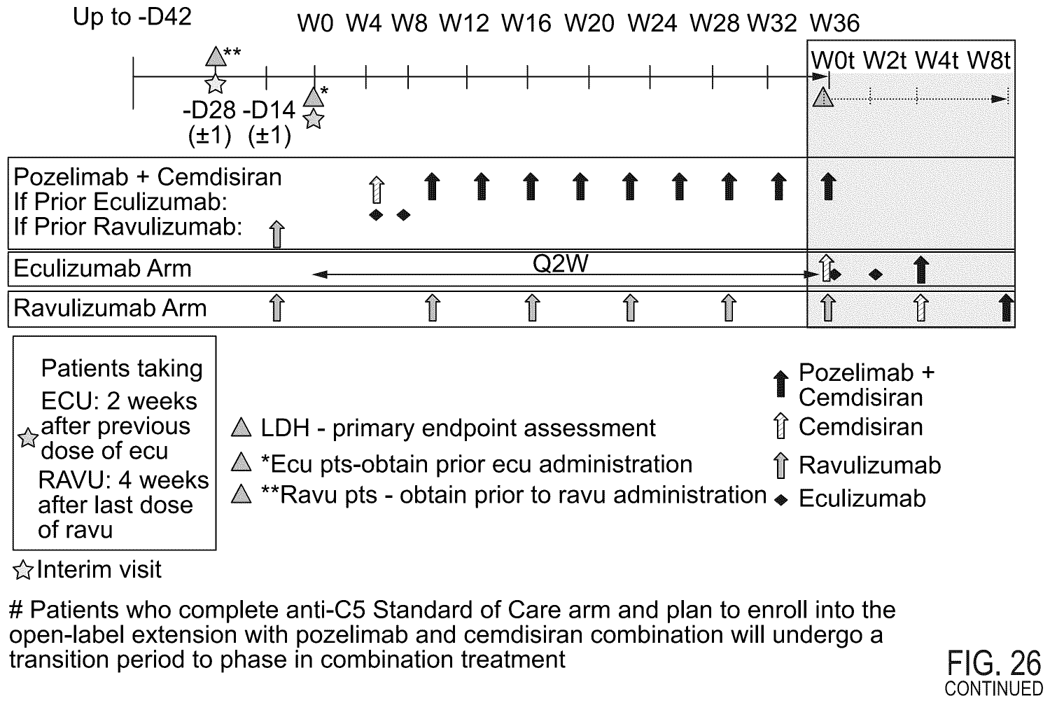

The study (FIG. 26) has the following periods: a 6-week screening period and a 36-week open label treatment period (OLTP). Patients who complete the OLTP in the anti-C5 standard-of-care arm and plan to enroll in the follow-on open-label long-term extension study with Pozelimab and Cemdisiran combination must participate in a post-OLTP transition period. Patients who discontinue study treatment as well as patients who decline enrollment into the OLE study will undergo a safety off-treatment follow-up period of up to 52 weeks.

### Screening Period.

The first screening visit should take place up to 6 weeks prior to day 1 (Table 6-1). Screening visit 1 should be scheduled based on the patient's Eculizumab or Ravulizumab dosing regimen prior to the study.

For patients taking Eculizumab at screening:
Screening visit 1 should be scheduled on the day of or the day prior to an Eculizumab dose and may take place up to 6 weeks prior to day 1.
At visit 2e, patients will be provided with an eCOA device to take home.
Day 1 shall be scheduled on the day of or up to 2 days prior to the patient's scheduled Eculizumab administration.

For patients taking Ravulizumab at screening:
Screening visit 1 should be scheduled about 6 weeks after a Ravulizumab dose
Visit 2r should be scheduled on the day of or up to 2 days prior to the patient's next scheduled Ravulizumab dose. This will be the last non-study Ravulizumab dose. Additional laboratory samples for baseline assessments will be taken at this visit and eCOA device will be provided to the patient to take home.
The day 1 visit should be scheduled 26 to 28 days after the last administration of Ravulizumab at visit 2r.

Additional interim screening visit(s) may take place as needed, for instance, for repeat blood collection.

Historical data will be collected such as but not limited to Eculizumab or Ravulizumab administration, concomitant medications, hemolytic parameters, and RBC transfusions.

Due to the risk of Neisseria meningitidis infection, patients will require administration of meningococcal vaccination(s) in accordance with the local Eculizumab or Ravulizumab prescribing information, where applicable, and in accordance with current national vaccination guidelines for vaccination use with complement inhibitors or local practice and at the very least, within a period of 5 years prior to screening. For patients who require administration with meningococcal vaccination(s) during the screening period, administration should occur preferably at least 2 weeks prior to day 1, or at another time point according to local practice or national guidelines/local Eculizumab or Ravulizumab prescribing information (as applicable). If vaccination precedes the initiation of study treatment by less than 2 weeks, then the patient must receive antibiotic prophylaxis for a minimum of 2 weeks from the date of vaccine administration.

In addition to Neisseria meningitidis infection, fatal or serious infections with Neisseria gonorrhea have been reported in patients taking complement inhibitor therapy. Patients should therefore undergo a risk assessment and counseling regarding the potential risk of Neisseria gonorrhea as per local practice or national guidelines.

Patients who have not been vaccinated against Streptococcus pneumoniae and Haemophilus influenzae type B may receive these vaccinations during the screening period or on the day of randomization, based on investigator discretion and taking into consideration the available national guidelines.

Patients will be assessed for active or latent tuberculosis (TB) infection based on local practice or applicable guidelines. Based on the risk assessment, the need for screening with either tuberculin skin test or T-cell interferon-gamma release assay will be made. The interpretation of these results, as applicable, will be made by the investigator. Further management and treatment of TB will be the responsibility of the investigator.

### Randomization

Day 1 (randomization) must take place on the day of the patient's scheduled Eculizumab administration or 4 weeks (i.e., 26 to 28 days) after the last administration of Ravulizumab, as applicable. If the day of randomization cannot be scheduled on the day of the patient's next Eculizumab dose or exactly 4 weeks after the last Ravulizumab dose, a window of 1 to 2 days is allowed such that the day of randomization may take place 1 to 2 days prior to the next scheduled Eculizumab dose or 26 to 28 days after the last Ravulizumab dose, as applicable.

Patients who fulfill all the eligibility criteria will be randomized in a 1:1 ratio to anti C5 standard of-care (i.e., continue existing treatment with Eculizumab or Ravulizumab) or treatment with Pozelimab and Cemdisiran.

Randomization will be stratified based on the criteria described herein:

Eligible patients will be randomized in a 1:1 ratio to receive either the combination treatment with SC Pozelimab 400 mg and Cemdisiran 200 mg Q4W or continue their anti C5 standard-of-care therapy with either Eculizumab 900 mg IV Q2W or IV Ravulizumab according to the labeled weight-based dosing algorithm according to a central randomization scheme provided by an Interactive Web Response System (IRWS) to the designated study pharmacist (or qualified designee). Randomization will be stratified according to the following factors:
The screening visit LDH value (≤1.5×ULN or >1.5×ULN) Note: Enrollment of patients who have a screening visit LDH level >1.5×ULN will be capped at 20% of the overall enrolled population.
RBC/whole blood transfusion within the past 1 year prior to randomization (yes/no)
Anti-C5 standard-of-care therapy taken at screening (Eculizumab versus Ravulizumab)

### Open-Label Treatment Period (OLTP)

The treatment period is 36 weeks. Treatment administration is based on a patient's PNH treatment prior to screening as well as their treatment assignment:
Pozelimab and Cemdisiran arm:
   Patients who were on Eculizumab at screening will receive the following during the study:

| | |
|---|---|
| Day 1 | Cemdisiran 200 mg SC, followed by a 2-hour observation period, and |
| | Eculizumab 900 mg IV (+2 days for Eculizumab, from day 1 to day 3) |
| Day 15 (week 2) | Eculizumab 900 mg IV (+2 days, from day 13 to 17) |
| Day 29 (week 4) | Pozelimab 60 mg/kg single IV loading dose. After a 30-minute observation |
| | period, this IV infusion is followed by Pozelimab 400 mg SC and |
| | Cemdisiran 200 mg SC, followed by a 2-hour observation period after the |
| | last study drug administration |
| Day 57 (week 8) | Pozelimab 400 mg SC and Cemdisiran 200 mg SC (+7 days) maintenance |
| | regimen, followed by a 2-hour observation period after the last study drug |
| | administration at day 57, and after day 57, Pozelimab 400 mg SC and |
| | Cemdisiran 200 mg SC Q4W (+7 days) |
| | Note that a 2-hour observation period will be instituted after the Pozelimab |
| | SC administration on day 85 (week 12). |
| | For subsequent administrations after the third administration of each study |
| | treatment, the observation period can be reduced to 30 minutes, provided |
| | the combination is felt to be well tolerated by the patient, per investigator's |
| | discretion. |

Patients who were on Ravulizumab at screening will receive the following during the study:

| | |
|---|---|
| Day 1 | Cemdisiran 200 mg SC (26 to 28 days after last administration of |
| | Ravulizumab), followed by a 2-hour observation period |
| Day 29 (week 4) | Pozelimab 60 mg/kg single IV loading dose. After a 30-minute observation |
| | period, this IV infusion is followed by Pozelimab 400 mg SC and |
| | Cemdisiran 200 mg SC, followed by a 2-hour observation period after the |
| | last study drug administration |
| Day 57 (week 8) | Pozelimab 400 mg SC and Cemdisiran 200 mg SC (+7 days) maintenance |
| | regimen, followed by a 2-hour observation period after the last study drug |
| | administration at day 57, and after day 57, Pozelimab 400 mg SC and |
| | Cemdisiran 200 mg SC Q4W (+7 days) |
| | Note that a 2-hour observation period will be instituted after the Pozelimab |
| | SC administration on day 85 (week 12). |
| | For subsequent administrations after the third administration of each study |
| | drug, the observation period can be reduced to 30 minutes, provided the |
| | combination is felt to be well tolerated by the patient, per investigator's |
| | discretion. |

Anti-C5 standard-of-care arm:
   Patients who were on Eculizumab at screening will continue to receive Eculizumab during the study:

| | |
|---|---|
| Day 1 | Eculizumab 900 mg IV Q2W (+2 days) |
| Day 15 (week 2) | Eculizumab 900 mg IV Q2W (±2 days) |

Patients who were on Ravulizumab at screening will continue to receive Ravulizumab during the study:

| | |
|---|---|
| Day 29 (week 4) | Ravulizumab IV Q8W per labeled |
| | weight-based dosage (±7 days) |

During IV infusion of Pozelimab, due to concern of potential IV infusion reactions, patients should be observed for at least 30 minutes after the infusion and prior to the administration of the first SC injections for Pozelimab and Cemdisiran.

A 2-hour observation period should be instituted following the first 3 administrations of Pozelimab and Cemdisiran, whether alone or in combination. For subsequent administrations after the third administration of each study treatment, the observation period can be reduced to 30 minutes, provided the combination is felt to be well tolerated by the patient, per investigator's discretion

While the overall goal of the proposed dosing regimen is to prevent hemolysis, the initiation of therapy is also designed to mitigate the potential for the formation of large drug-target-drug (DTD) immune complexes of eculizumab-C5-pozelimab or ravulizumab-C5-pozelimab during the treatment switch. As explained herein, a lead-in cemdisiran dose plus 60 mg/kg IV loading dose of pozelimab is included in order to minimize large DTD immune complex formation.

### Post-Open-Label Treatment Period (Post-OLTP)

Patients who complete the OLTP (i.e., end of study [EOS] visit at day 253) will be offered the opportunity to enroll in another study, a follow-on OLE study. Screening for the next study may be conducted while the patient is in the OLTP, as described in Section 9.1.1.1 footnote #1.

For patients who complete the 36-week OLTP on the Pozelimab and Cemdisiran arm, the transition of treatment from the current study to the OLE is planned to be uninterrupted, whereby day 1 visit of the OLE will correspond to the EOT period/EOS visit in the current study.

Patients who complete the 36-week OLTP on the anti-C5 standard-of-care arm and plan to participate in the OLE study will undergo a transition period in order to switch to Cemdisiran and Pozelimab combination treatment in the same manner as those switching to the combination at study initiation as follows:

Patients treated with Eculizumab:
Transition day 1 (week 36 of OLTP): Cemdisiran 200 mg SC, followed by a 2-hour observation period, and Eculizumab 900 mg IV (±2 days treatment window)
Transition day 15 (week 2 of transition period): Eculizumab 900 mg IV (±2 days)
Transition day 29 (week 4 of transition period): Pozelimab 60 mg/kg IV loading dose. After a 30-minute observation period, this IV infusion is followed by Pozelimab 400 mg SC and Cemdisiran 200 mg SC, followed by a 2-hour observation period after the last study drug administration. Patients will transition to the OLE study after completing the week 4t visit.

Patients treated with Ravulizumab:
Transition day 1 (week 36 of OLTP): IV Ravulizumab at the labeled weight-based dosage
Transition day 29 (week 4 of transition period): Cemdisiran 200 mg SC, followed by a 2-hour observation period
Transition day 57 (week 8 of transition period): Pozelimab 60 mg/kg IV loading dose. After a 30-minute observation period, this IV infusion is followed by Pozelimab 400 mg SC and Cemdisiran 200 mg SC, followed by a 2-hour observation period after the last study drug administration. Patients will transition to the OLE study after completing the week 8t visit.

This approach for patients randomized to the anti-C5 standard-of-care arm and who plan to enroll in the OLE study, ensures a consistent dosing approach when switching from Eculizumab or Ravulizumab to Pozelimab and Cemdisiran combination therapy and has no impact on study efficacy analysis for the randomized treatment period. Patients who discontinue treatment as well as patients who decline enrollment into the follow-on study of the OLE will undergo a safety off-treatment follow up period (FUP) of up to 52 weeks according to Table 6-4. Patients discontinuing the study treatment should be treated in accordance with local standards of care while continuing to be monitored in the 52-week off-treatment safety FUP. Investigators switching patients from the combination treatment to another anti-C5 mAb should have a heightened awareness for possible adverse events resulting from the formation of large DTD immune complexes.

The main study is considered finished, when all patients either complete the 36-week treatment period or prematurely discontinue the study. Additional data collected during the transition period and safety off-treatment FUP will be described separately.

Study assessments and procedures are presented by study period and visit in Table 6-1 (OLTP), Table 6-2 (transition period for Eculizumab arm), Table 6-3 (transition period for Ravulizumab arm), and Table 6-4 (FUP). See FIG. 26.

TABLE 6-1
Schedule of Events for the Open-Label Treatment Period

| | | Study Procedure (Visit)¹ | | | | |
|---|---|---|---|---|---|---|
| | | Screening | | | | |
| | | | For | For | | |
| | | | ravu | ecu | Open Label Treatment Period (OLTP) | |
| | | Visit # | | | | |
| | | V1² | V2r | V2e | V3 | V4 |
| | | Week | | | | |
| | | Up | | −4 | | |
| | | to −6 | −4 | to −2 | 0 | 2 |
| | | Day | | | | |
| | | Up | | −28 | | |
| | | to −42 | −28 | to −14 | 1³ | 15 |
| | | Window (day) | | | | |
| | | | +2 | | | ±2 |
| Screening/Baseline²: | | | | | | |
| eCOA device dispensation | | | X | X | | |
| Inclusion/Exclusion | | X | X | X | X | |
| Informed consent | | X | | | | |
| FBR informed consent (optional) | | X | | | | |
| Genomics informed consent (optional) | | X | | | | |
| Medical history⁴ | | X | | | | |
| Prior medications⁵ | | X | | | | |
| Demographics | | X | | | | |
| Height | | X | | | | |
| Hepatitis B and C testing | | X | | | | |
| Vaccination/revaccinate for Neisseria | | <-------------- X ----------> | | | | |
| meningitidis⁶ | | | | | | |
| Vaccination against Streptococcus | | <-------------- X ----------> | | | | |
| pneumoniae and Haemophilus influenzae | | | | | | |
| type B (if needed) ⁷ | | | | | | |
| Tuberculosis history and assessment⁸ | | X | | | | |
| Risk assessment for Neisseria gonorrhea⁹ | | X | | | | |
| Randomization | | | | | X | |
| Treatment: | | | | | | |
| IVRS/IWRS | | X | | | X | X |
| Timing of | Administer Eculizumab 900 mg | X | <---- Q2W ---> | | | |
| ecu/ravu | IV Q2W (ONLY patients | | | | | |
| (non-IMP) | already receiving Eculizumab) | | | | | |
| during | Administer Ravulizumab IV | | X³⁶ | | | |
| screening | Q8W according to labeled | | | | | |
| | weight-based dosing (ONLY | | | | | |
| | patients already receiving | | | | | |
| | Ravulizumab) | | | | | |
| Pozelimab | Administer Eculizumab 900 mg | | | | X¹¹ | X¹¹ |
| and | IV | | | | | |
| Cemdisiran | (This is non-IMP -- ONLY for | | | | | |
| arm | patients previously taking | | | | | |
| | Eculizumab) | | | | | |
| | Administer Pozelimab 60 | | | | | |
| | mg/kg IV loading dose | | | | | |
| | Administer Cemdisiran 200 mg | | | | X | |
| | SC Q4W^{13, 14} | | | | | |
| | Administer Pozelimab 400 mg | | | | | |
| | SC Q4W^{12, 14} | | | | | |
| Anti-C5 | Administer Eculizumab 900 mg | | | | X | X |
| SOC: | IV Q2W¹⁵ | | | | | |
| Eculizumab | Administer Cemdisiran 200 mg | | | | | |
| IMP arm | SC (ONLY if enrolling in the | | | | | |
| | OLE) | | | | | |
| Anti-C5 | Administer Ravulizumab IV | | | | | |
| SOC: | Q8W according to labeled | | | | | |
| Ravulizumab | weight based dosing¹⁶ | | | | | |
| IMP arm | | | | | | |

| | | | | | |
|---|---|---|---|---|---|
| Concomitant meds and treatment | X | X | X | X | X |
| Transfusion record update | X | X | X | X | X |

| | |
|---|---|
| Antibiotics prophylaxis (recommended)¹⁹ | <-----------------------------------X----------------------> |

Clinical outcome assessments (COAs):

| FACIT-Fatigue | | | | | X | X |
|---|---|---|---|---|---|---|
| EORTC-QLQ-C30 | | | | | X | X |
| EQ-5D-5L | | | | | X | |
| TSQM | | | | | X | |
| PNH symptom-specific questionnaire (daily | | | <---------------------------------------X-----------------------------------------> | | | |
| from day −14 to end of study) ²⁰ | | | | | | |
| PGIS (PNH Symptoms/Impacts/Fatigue) | | | | | X | X |
| PGIC (PNH Symptoms/Impacts/Fatigue) | | | | | | |
| Safety and Anthropometric: | | | | | | |
| Patient safety card for Neisseria | | | | | X | X |
| meningitidis¹⁰ | | | | | | |
| For Ravulizumab arm only: Provide patient | | | | | X | |
| safety brochure for Ravulizumab | | | | | | |
| For Eculizumab arm only: Provide patient | | | | | X | |
| safety brochure for Eculizumab | | | | | | |
| Body weight | | X | | | | |
| Vital signs²¹ | | X | | | X | X |
| Physical examination²² | | X | | | | |
| Electrocardiogram | | X | | | | |
| Adverse events | | X | X | X | X | X |
| Breakthrough hemolysis assessment²³ | | X | X | X | X | X |
| Laboratory Testing²⁴: | | | | | | |
| Titers to measure N. meningitidis (only if | | X | | | | |
| required per local practice/regulations) | | | | | | |
| For patients receiving | Blood chemistry | | X | | | |
| Ravulizumab prior to | Hematology | | X | | | |
| screening: Additional | Coagulation panel | | X | | | |
| samples | Free hemoglobin | | X | | | |
| (drawn pre-ravu dose) | Haptoglobin | | X | | | |

| | | | |
|---|---|---|---|
| Hematology²⁵ | X | X | X |
| Coagulation panel | X | X | X |
| Blood chemistry (long panel) including | X | X | X |
| LDH²⁶ | | | |
| D-dimer | | X | |
| Immunoglobulin G | | X | |
| Pregnancy test (applicable patients)²⁷ | X | X | |
| Urinalysis | X | X | X |
| Direct antiglobulin test (DAT or Coombs | | X | |
| test) | | | |

Pharmacokinetics, ADA and Total C5 Sampling²⁸:

| | | | |
|---|---|---|---|
| Pozelimab | Blood samples for conc. of | X | |
| and | Pozelimab | | |
| Cemdisiran | Blood samples for conc. of | X | |
| arm | Cemdisiran and metabolites³⁰ | | |
| | Blood samples for ADA of | X | |
| | Pozelimab³¹ | | |
| | Blood samples for ADA of | X | |
| | Cemdisiran³¹ | | |
| | Blood samples for conc. of total | X | X |
| | C5 | | |
| Anti-C5 | Blood samples for conc. of | X | X |
| standard- | Eculizumab (for patients taking | | |
| of-care arm | Eculizumab only) | | |
| | Blood samples for conc. of | X | X |
| | Ravulizumab (for patients | | |
| | taking Ravulizumab only) | | |
| | Blood samples for conc. of | | |
| | Cemdisiran and metabolites³⁰ | | |
| | Blood samples for ADA of | | |
| | Pozelimab³¹ | | |
| | Blood samples for ADA of | | |
| | Cemdisiran³¹ | | |
| | Blood samples for conc. of total | X | X |
| | C5 | | |

| | | | |
|---|---|---|---|
| Biomarkers: | | | |
| Free hemoglobin²⁸ | | X³⁴ | |
| Haptoglobin²⁸ | | X³⁴ | |
| Complement hemolytic assay (serum CH50 | | X³⁴ | X |
| & AH50)²⁸ | | | |
| sC5b-9 (plasma)²⁸ | | X³⁴ | |
| PNH erythrocyte cells²⁸ | X | | |
| PNH granulocytes and monocytes²⁸ | X | | |
| Exploratory research serum sample²⁸ | | X³⁴ | |
| Exploratory research plasma sample²⁸ | | X³⁴ | X |
| Optional pharmacogenomics (DNA AND RNA): | | | |
| Whole blood for DNA isolation (optional)³⁵ | | X | |
| Whole blood for RNA isolation (optional) | | X | |

| | | Study Procedure (Visit)¹ | | | | | | | |
|---|---|---|---|---|---|---|---|---|---|
| | | Open Label Treatment Period (OLTP) | | | | | | | |
| | | Visit # | | | | | | | |
| | | V5 | V6 | V7 | V8 | V9 | V10 | V11 | V12 |
| | | Week | | | | | | | |
| | | 4 | 6 | 8 | 10 | 12 | 16 | 20 | 24 |
| | | Day | | | | | | | |
| | | 29 | 43 | 57 | 71 | 85 | 113 | 141 | 169 |
| | | Window (day) | | | | | | | |
| | | ±3 | ±3 | ±3 | ±3 | ±3 | ±7 | ±7 | ±7 |
| Screening/Baseline²: | | | | | | | | | |
| eCOA device dispensation | | | | | | | | | |
| Inclusion/Exclusion | | | | | | | | | |
| Informed consent | | | | | | | | | |
| FBR informed consent (optional) | | | | | | | | | |
| Genomics informed consent (optional) | | | | | | | | | |
| Medical history⁴ | | | | | | | | | |
| Prior medications⁵ | | | | | | | | | |
| Demographics | | | | | | | | | |
| Height | | | | | | | | | |
| Hepatitis B and C testing | | | | | | | | | |
| Vaccination/revaccinate for Neisseria | | | | | | | | | |
| meningitidis⁶ | | | | | | | | | |
| Vaccination against Streptococcus | | | | | | | | | |
| pneumoniae and Haemophilus influenzae | | | | | | | | | |
| type B (if needed) ⁷ | | | | | | | | | |
| Tuberculosis history and assessment⁸ | | | | | | | | | |
| Risk assessment for Neisseria gonorrhea⁹ | | | | | | | | | |
| Randomization | | | | | | | | | |
| Treatment: | | | | | | | | | |
| IVRS/IWRS | | X | X | X | X | X | X | X | X |
| Timing of | Administer Eculizumab 900 mg | | | | | | | | |
| ecu/ravu | IV Q2W (ONLY patients | | | | | | | | |
| (non-IMP) | already receiving Eculizumab) | | | | | | | | |
| during | Administer Ravulizumab IV | | | | | | | | |
| screening | Q8W according to labeled | | | | | | | | |
| | weight-based dosing (ONLY | | | | | | | | |
| | patients already receiving | | | | | | | | |
| | Ravulizumab) | | | | | | | | |
| Pozelimab | Administer Eculizumab 900 mg | | | | | | | | |
| and | IV | | | | | | | | |
| Cemdisiran | (This is non-IMP -- ONLY for | | | | | | | | |
| arm | patients previously taking | | | | | | | | |
| | Eculizumab) | | | | | | | | |
| | Administer Pozelimab 60 | X¹² | | | | | | | |
| | mg/kg IV loading dose | | | | | | | | |
| | Administer Cemdisiran 200 mg | X | | X | | X | X | X | X |
| | SC Q4W^{13, 14} | | | | | | | | |
| | Administer Pozelimab 400 mg | | | | | X | X | X | X |
| | SC Q4W^{12, 14} | | | | | | | | |
| Anti-C5 | Administer Eculizumab 900 mg | X | X | X | X | X | X | X | X |
| SOC: | IV Q2W¹⁵ | | | | | | | | |
| Eculizumab | Administer Cemdisiran 200 mg | | | | | | | | |
| IMP arm | SC (ONLY if enrolling in the | | | | | | | | |
| | OLE) | | | | | | | | |
| Anti-C5 | Administer Ravulizumab IV | X | | | | X | | X | |
| SOC: | Q8W according to labeled | | | | | | | | |
| Ravulizumab | weight based dosing¹⁶ | | | | | | | | |
| IMP arm | | | | | | | | | |

| | | | | | | | | |
|---|---|---|---|---|---|---|---|---|
| Concomitant meds and treatment | X | X | X | X | X | X | X | X |
| Transfusion record update | X | X | X | X | X | X | X | X |

| | |
|---|---|
| Antibiotics prophylaxis (recommended)¹⁹ | <-----------------------------------X----------------------> |

Clinical outcome assessments (COAs):

| FACIT-Fatigue | | X | | X | | X | X | X | X |
|---|---|---|---|---|---|---|---|---|---|
| EORTC-QLQ-C30 | | X | | X | | X | X | X | X |
| EQ-5D-5L | | | | | | | | | |
| TSQM | | X | | X | | X | X | X | X |
| PNH symptom-specific questionnaire (daily | | <---------------------------------------X-----------------------------------------> | | | | | | | |
| from day −14 to end of study) ²⁰ | | | | | | | | | |
| PGIS (PNH Symptoms/Impacts/Fatigue) | | X | | | | X | | X | |
| PGIC (PNH Symptoms/Impacts/Fatigue) | | X | | | | X | | | |
| Safety and Anthropometric: | | | | | | | | | |
| Patient safety card for Neisseria | | X | X | X | X | X | X | X | X |
| meningitidis¹⁰ | | | | | | | | | |
| For Ravulizumab arm only: Provide patient | | | | | | | | | |
| safety brochure for Ravulizumab | | | | | | | | | |
| For Eculizumab arm only: Provide patient | | | | | | | | | |
| safety brochure for Eculizumab | | | | | | | | | |
| Body weight | | X | | X | | X | X | X | X |
| Vital signs²¹ | | X | X | X | X | X | X | X | X |
| Physical examination²² | | X | | X | | X | | | |
| Electrocardiogram | | | | | | X | | | |
| Adverse events | | X | X | X | X | X | X | X | X |
| Breakthrough hemolysis assessment²³ | | X | X | X | X | X | X | X | X |
| Laboratory Testing²⁴: | | | | | | | | | |
| Titers to measure N. meningitidis (only if | | | | | | | | | |
| required per local practice/regulations) | | | | | | | | | |
| For patients receiving | Blood chemistry | | | | | | | | |
| Ravulizumab prior to | Hematology | | | | | | | | |
| screening: Additional | Coagulation panel | | | | | | | | |
| samples | Free hemoglobin | | | | | | | | |
| (drawn pre-ravu dose) | Haptoglobin | | | | | | | | |

| | | | | | | | | |
|---|---|---|---|---|---|---|---|---|
| Hematology²⁵ | X | X | X | X | X | X | X | X |
| Coagulation panel | X | X | X | X | X | X | X | X |
| Blood chemistry (long panel) including | X | X | X | X | X | X | X | X |
| LDH²⁶ | | | | | | | | |
| D-dimer | | | | | | X | | |
| Immunoglobulin G | X | | | | X | | | |
| Pregnancy test (applicable patients)²⁷ | X | | X | | X | X | X | X |
| Urinalysis | X | | | | | | | |
| Direct antiglobulin test (DAT or Coombs | X | | X | | X | X | X | X |
| test) | | | | | | | | |

Pharmacokinetics, ADA and Total C5 Sampling²⁸:

| | | | | | | | |
|---|---|---|---|---|---|---|---|
| Pozelimab | Blood samples for conc. of | X²⁹ | X | X | X | X | X |
| and | Pozelimab | | | | | | |
| Cemdisiran | Blood samples for conc. of | | | X | | | X |
| arm | Cemdisiran and metabolites³⁰ | | | | | | |
| | Blood samples for ADA of | | | X | | | X |
| | Pozelimab³¹ | | | | | | |
| | Blood samples for ADA of | | | X | | | X |
| | Cemdisiran³¹ | | | | | | |
| | Blood samples for conc. of total | X | X | X | X | X | X |
| | C5 | | | | | | |
| Anti-C5 | Blood samples for conc. of | X | | X | | | X |
| standard- | Eculizumab (for patients taking | | | | | | |
| of-care arm | Eculizumab only) | | | | | | |
| | Blood samples for conc. of | X | | X | | | |
| | Ravulizumab (for patients | | | | | | |
| | taking Ravulizumab only) | | | | | | |
| | Blood samples for conc. of | | | | | | |
| | Cemdisiran and metabolites³⁰ | | | | | | |
| | Blood samples for ADA of | | | | | | |
| | Pozelimab³¹ | | | | | | |
| | Blood samples for ADA of | | | | | | |
| | Cemdisiran³¹ | | | | | | |
| | Blood samples for conc. of total | X | X | X | X | X | X |
| | C5 | | | | | | |

| | | | | | | | |
|---|---|---|---|---|---|---|---|
| Biomarkers: | | | | | | | |
| Free hemoglobin²⁸ | X | | X | | X | | X |
| Haptoglobin²⁸ | X | | X | | X | | X |
| Complement hemolytic assay (serum CH50 | X | X | X | X | X | | X |
| & AH50)²⁸ | | | | | | | |
| sC5b-9 (plasma)²⁸ | X | | | | | | |
| PNH erythrocyte cells²⁸ | | | | | | | |
| PNH granulocytes and monocytes²⁸ | | | | | | X | |
| Exploratory research serum sample²⁸ | | X | | | | | |
| Exploratory research plasma sample²⁸ | | X | X | X | | | |
| Optional pharmacogenomics (DNA AND RNA): | | | | | | | |
| Whole blood for DNA isolation (optional)³⁵ | | | | | | | |
| Whole blood for RNA isolation (optional) | | | | | X | | |

| | | Study Procedure (Visit)¹ | | |
|---|---|---|---|---|
| | | Open Label Treatment Period (OLTP) | | |
| | | | | EOS |
| | | Visit # | | |
| | | V13 | V14 | V15 |
| | | Week | | |
| | | 28 | 32 | 36 |
| | | Day | | |
| | | 197 | 225 | 253 |
| | | Window (day) | | |
| | | ±7 | ±3 | ±3 |
| Screening/Baseline²: | | | | |
| eCOA device dispensation | | | | |
| Inclusion/Exclusion | | | | |
| Informed consent | | | | |
| FBR informed consent (optional) | | | | |
| Genomics informed consent (optional) | | | | |
| Medical history⁴ | | | | |
| Prior medications⁵ | | | | |
| Demographics | | | | |
| Height | | | | |
| Hepatitis B and C testing | | | | |
| Vaccination/revaccinate for Neisseria | | | | |
| meningitidis⁶ | | | | |
| Vaccination against Streptococcus | | | | |
| pneumoniae and Haemophilus influenzae | | | | |
| type B (if needed) ⁷ | | | | |
| Tuberculosis history and assessment⁸ | | | | |
| Risk assessment for Neisseria gonorrhea⁹ | | | | |
| Randomization | | | | |
| Treatment: | | | | |
| IVRS/IWRS | | X | X | X |
| Timing of | Administer Eculizumab 900 mg | | | |
| ecu/ravu | IV Q2W (ONLY patients | | | |
| (non-IMP) | already receiving Eculizumab) | | | |
| during | Administer Ravulizumab IV | | | |
| screening | Q8W according to labeled | | | |
| | weight-based dosing (ONLY | | | |
| | patients already receiving | | | |
| | Ravulizumab) | | | |
| Pozelimab | Administer Eculizumab 900 mg | | | |
| and | IV | | | |
| Cemdisiran | (This is non-IMP -- ONLY for | | | |
| arm | patients previously taking | | | |
| | Eculizumab) | | | |
| | Administer Pozelimab 60 | | | |
| | mg/kg IV loading dose | | | |
| | Administer Cemdisiran 200 mg | X | X | X³⁷ |
| | SC Q4W^{13, 14} | | | |
| | Administer Pozelimab 400 mg | X | X | X³⁷ |
| | SC Q4W^{12, 14} | | | |
| Anti-C5 | Administer Eculizumab 900 mg | X | X | X³⁷ |
| SOC: | IV Q2W¹⁵ | | | |
| Eculizumab | Administer Cemdisiran 200 mg | | | X³⁷ |
| IMP arm | SC (ONLY if enrolling in the | | | |
| | OLE) | | | |
| Anti-C5 | Administer Ravulizumab IV | X | | X³⁷ |
| SOC: | Q8W according to labeled | | | |
| Ravulizumab | weight based dosing¹⁶ | | | |
| IMP arm | | | | |
| Concomitant meds and treatment | | X | X | X |
| Transfusion record update | | X | X | X |
| Antibiotics prophylaxis (recommended)¹⁹ | | <-----------------------------------X----------------------> | | |
| Clinical outcome assessments (COAs): | | | | |
| FACIT-Fatigue | | X | X | X |
| EORTC-QLQ-C30 | | X | X | X |
| EQ-5D-5L | | | | X |
| TSQM | | X | X | X |
| PNH symptom-specific questionnaire (daily | | <---------------------------------------X-----------------------------------------> | | |
| from day −14 to end of study) ²⁰ | | | | |
| PGIS (PNH Symptoms/Impacts/Fatigue) | | X | | X |
| PGIC (PNH Symptoms/Impacts/Fatigue) | | | | X |
| Safety and Anthropometric: | | | | |
| Patient safety card for Neisseria | | X | X | X |
| meningitidis¹⁰ | | | | |
| For Ravulizumab arm only: Provide patient | | | | |
| safety brochure for Ravulizumab | | | | |
| For Eculizumab arm only: Provide patient | | | | |
| safety brochure for Eculizumab | | | | |
| Body weight | | X | X | X |
| Vital signs²¹ | | X | X | X |
| Physical examination²² | | X | | X |
| Electrocardiogram | | | | X |
| Adverse events | | X | X | X |
| Breakthrough hemolysis assessment²³ | | X | X | X |
| Laboratory Testing²⁴: | | | | |
| Titers to measure N. meningitidis (only if | | | | |
| required per local practice/regulations) | | | | |
| For patients receiving | Blood chemistry | | | |
| Ravulizumab prior to | Hematology | | | |
| screening: Additional | Coagulation panel | | | |
| samples | Free hemoglobin | | | |
| (drawn pre-ravu dose) | Haptoglobin | | | |
| Hematology²⁵ | | X | X | X |
| Coagulation panel | | X | X | X |
| Blood chemistry (long panel) including | | X | X | X |
| LDH²⁶ | | | | |
| D-dimer | | | | X |
| Immunoglobulin G | | | | |
| Pregnancy test (applicable patients)²⁷ | | X | X | X |
| Urinalysis | | | | |
| Direct antiglobulin test (DAT or Coombs | | | X | X |
| test) | | | | |
| Pharmacokinetics, ADA and Total C5 Sampling²⁸: | | | | |
| Pozelimab | Blood samples for conc. of | X | X | X |
| and | Pozelimab | | | |
| Cemdisiran | Blood samples for conc. of | | X | |
| arm | Cemdisiran and metabolites³⁰ | | | |
| | Blood samples for ADA of | | | X |
| | Pozelimab³¹ | | | |
| | Blood samples for ADA of | | | X |
| | Cemdisiran³¹ | | | |
| | Blood samples for conc. of total | X | X | X |
| | C5 | | | |
| Anti-C5 | Blood samples for conc. of | | | X |
| standard- | Eculizumab (for patients taking | | | |
| of-care arm | Eculizumab only) | | | |
| | Blood samples for conc. of | X | | X |
| | Ravulizumab (for patients | | | |
| | taking Ravulizumab only) | | | |
| | Blood samples for conc. of | | | X³² |
| | Cemdisiran and metabolites³⁰ | | | |
| | Blood samples for ADA of | | | X³³ |
| | Pozelimab³¹ | | | |
| | Blood samples for ADA of | | | X³³ |
| | Cemdisiran³¹ | | | |
| | Blood samples for conc. of total | X | X | X |
| | C5 | | | |

| | | | |
|---|---|---|---|
| Biomarkers: | | | |
| Free hemoglobin²⁸ | X | | X |
| Haptoglobin²⁸ | X | | X |
| Complement hemolytic assay (serum CH50 | X | | X |
| & AH50)²⁸ | | | |
| sC5b-9 (plasma)²⁸ | | | X |
| PNH erythrocyte cells²⁸ | | | |
| PNH granulocytes and monocytes²⁸ | | | X |
| Exploratory research serum sample²⁸ | X | | X |
| Exploratory research plasma sample²⁸ | X | X | X |
| Optional pharmacogenomics (DNA AND RNA): | | | |
| Whole blood for DNA isolation (optional)³⁵ | | | |
| Whole blood for RNA isolation (optional) | | | X |

Footnotes
¹When multiple procedures are performed on the same day, the sequence of procedures is as follows: Clinical outcome assessments, electrocardiogram (ECG) and/or vital signs, blood collection, and study treatment administration. Patients who are being screened in this study (R3918-PNH-2022) who complete the R3918-PNH-2021 study will have all assessments performed as indicated in the SOE. This implies that the Screening Visit 1 in the R3918-PNH-2022 study may take place at week 24 of the R3918-PNH-2021 study and V2r of the R3918-PNH-2022 study take place at the time of week 26 of theR3918-PNH-2021 study prior to the Ravulizumab dose administration. Assessments that are common to both studies should not be duplicated.
²Screening visit 1 should be scheduled based on the patient's dosing regimen prior to the study. For patients taking Eculizumab, screening visit 1 should be scheduled on the day of or the day prior to an Eculizumab dose. For patients taking Ravulizumab, screening visit 1 should be scheduled about 6 weeks after the last Ravulizumab dose. Additional screening visits may be scheduled as needed.
³For patients taking Ravulizumab, the day 1 visit should occur 4 weeks (i.e., 26 to 28 days) after the last administration of Ravulizumab.
⁴Medical history including, transfusions, breakthrough hemolysis history, and laboratory parameters for measurement of hemolysis (such as LDH, bilirubin, haptoglobin, reticulocyte count, and hemoglobin) should be obtained for the past 52 weeks, if possible. Prior history of thrombosis and infections of the Neisseria spp. will be collected. Patients who have a C5 mutation confirmed while the study is ongoing should have the information included as part of the patient's medical history. Patients who are poor responders to Eculizumab or Ravulizumab treatment during the study may be asked for a mutation analysis to be conducted as part of the study, if the patient agrees to such testing.
⁵Including detailed Eculizumab or Ravulizumab administration history and Neisseria meningitidis vaccination and other vaccinations as applicable.
⁶Patients will require administration with meningococcal vaccination unless documentation is provided of prior immunization in the past 5 years, or less than 5 years if required according to current national vaccination guidelines for vaccination use with complement inhibitors/local Eculizumab or Ravulizumab prescribing information. For patients who require administration with meningococcal vaccination(s) during the screening period, administration should occur preferably at least 2 weeks prior to day 1, or at another time point according to local Eculizumab or Ravulizumab prescribing information/national guidelines.
⁷Vaccination for Streptococcus pneumoniae and Haemophilus influenzae type B should be initiated per current national/local vaccination guidelines.
⁸Tuberculosis history and assessment: Screening by tuberculin skin test or T-cell interferon gamma release assay may be performed according to local practice or guidelines at the discretion of the investigator
⁹A risk factor assessment for Neisseria gonorrhea will be performed in accordance with local practice/national guidelines, and regular testing and counseling is advised for at-risk patients.
¹⁰Patient safety card: provide the patient safety card for Neisseria meningitidis infection to the patient on day 1 and any other visit when needed. Site should review the instructions on the safety card with the patient at each visit.
¹¹Eculizumab may be administered up to 2 days after day 1 visit or within 2 days of day 15 visit. If Eculizumab administration does not coincide with the day of clinic visit, as applicable, then the clinic visit should always precede the infusion of Eculizumab for patients on Q2W Eculizumab dosing.
¹²Pozelimab administration: administer study treatment to patients previously taking Eculizumab or Ravulizumab and randomized to Pozelimab/Cemdisiran arm.
¹³Cemdisiran administration: administer study treatment to patients randomized to Pozelimab/Cemdisiran arm.
¹⁴The dose of Pozelimab and Cemdisiran should be given Q4W (every 28 days) and on the day of the corresponding study visit whenever possible. Study treatment administration should always be the last procedure after all blood sample collection and study assessments have been completed. If Pozelimab or Cemdisiran cannot be administered on the day of the corresponding study visit, the combination may be administered up to 7 days before or up to 7 days after the planned dosing date as long as the dosing takes place after the corresponding study visit has been completed. For example, the week 8 (day 57) visit can take place from day 54 to day 60 given the visit window. The dose of Pozelimab and Cemdisiran therefore can be given from day 54 to day 64 but only on or after the week 8 visit assessments have been performed. Similarly, the week 16 (day 113) visit can take place from day 106 to day 120 given the visit window. The dose of Pozelimab and Cemdisiran can be given from day 106 to day 120 but only on or after the week 16 visit assessments have been performed. Pozelimab and Cemdisiran should be administered on the same day whenever possible. A 2-hour observation period should be instituted following the first 3 administrations of Pozelimab and Cemdisiran, whether alone or in combination. For subsequent administrations after the third administration of each study treatment, the observation period can be reduced to 30 minutes, provided the combination is felt to be well tolerated by the patient, per investigator's discretion.
¹⁵Eculizumab administration: administer Eculizumab study treatment to patients previously taking Eculizumab and randomized to the anti-C5 standard-of-care arm. Time points at weeks 14, 18, 22, 26, 30, and 34 are not included in the schedule of events, nevertheless Eculizumab should be administered at these time points. If Eculizumab administration does not coincide with the day of clinic visit, as applicable, then the clinic visit should always precede the infusion of Eculizumab. Patients who opt to enroll in the OLE should follow the schedule of events for the transition period (Table 6-2) after completion of the end of OLTP.
¹⁶Only patients who plan to continue in the follow-on OLE study will receive a dose of study treatment at the EOS week 36 visit for the OLTP. In addition, patients who are taking Eculizumab or Ravulizumab during the OLTP should continue to follow the schedule of events for the transition period (Table 6-2) after completion of the end of OLTP.
Note:
Patients who completed the OLTP but who decline enrollment into the OLE study will not receive study treatment at the EOS week 36 visit and should continue to follow the schedule of events for the follow-up period (Table 6-4).
¹⁷Intentionally left blank
¹⁸Intentionally left blank
¹⁹Daily oral antibiotic prophylaxis against Neisseria meningitidis is recommended starting on the first day of dosing with study treatment and continuing until up to 52 weeks after discontinuation of Pozelimab/Cemdisiran. For post-treatment prophylaxis for Eculizumab or Ravulizumab follow the local prescribing information/national guidelines/local practice. If vaccination for Neisseria meningitidis occurs less than 2 weeks prior to day 1, then antibiotic prophylaxis must be administered for at least 2 weeks from the day of vaccination.
²⁰Patient will complete PNH Symptom-Specific Questionnaire daily for 14 days prior to day 1 visit and continuing through the OLTP.
²¹Vital signs include temperature, sitting blood pressure, and pulse. Vital signs will be obtained pre-dose after the patient has been sitting quietly for at least approximately 5 minutes, where applicable.
²²Physical examination will include an evaluation of the head and neck, lungs, heart, abdomen, extremities, and skin. Care should be taken to examine and assess any abnormalities that may be present, as indicated by the patient's medical history.
²³Breakthrough hemolysis assessment: If a patient is suspected of having a breakthrough hemolysis event, then in addition to the required laboratory collection, additional samples for CBC, coagulation parameters (including D-dimer), chemistry, reticulocyte count, total C5, CH50, drug concentrations of Pozelimab/Cemdisiran/Eculizumab/Ravulizumab (depending on the patient's randomization/enrollment), ADA (against Pozelimab), and exploratory research serum and plasma will be collected unless already noted in the schedule of events for that visit. If the suspected event does not occur at a scheduled visit then an unscheduled visit should occur with an evaluation of the patient and collection of CBC, coagulation parameters (including D-dimer), chemistry, reticulocyte count, total C5, CH50, drug concentrations of Pozelimab/Cemdisiran/Eculizumab/Ravulizumab, and ADA (against Pozelimab), as applicable, and exploratory research serum and plasma.
²⁴During lab collection, handling and processing, the same methodology will be applied across study visits, as best as possible, to preserve the quality of sample and avoid hemolysis during sample processing. If the investigator or sponsor suspects that the lab result is not an accurate reflection of the patient's condition, consideration should be given to repeat the lab sample if clinically warranted, and in all cases where an LDH is ≥2 × ULN in association with a potassium >6 mmol/L. Blood collection should always be obtained prior to study treatment administration, unless otherwise noted. The coagulation blood sample must always be collected first, followed immediately by the blood chemistry sample.
²⁵Hemoglobin will be assessed as part of the hematology analysis. Hematology sample should be collected before study treatment administration.
²⁶Serum LDH, CRP, and bilirubin will be assessed as part of the blood chemistry analysis. During the screening period, obtain chemistry including LDH on the day of (or if not possible, one day before) Eculizumab or Ravulizumab administration. On day 1 and all subsequent visits, obtain chemistry including LDH prior to any study treatment administration, as applicable.
²⁷Pregnancy test: A serum test will be done at screening visit and a urine test will be done at all other visits.
²⁸Blood sample collection for concentrations of Pozelimab, Cemdisiran, Eculizumab, Ravulizumab, total C5, CH50, AH50, free hemoglobin, haptoglobin, sC5b-9, PNH erythrocytes, granulocytes, monocytes, and exploratory research serum and plasma sample: Obtain samples prior to any study drug administration (pre-dose). Sampling for PK and ADA is applicable to treatment received (i.e., drug conc for Pozelimab is obtained only in patients who receive Pozelimab). Eculizumab PK samples should be obtained in all patients taking Eculizumab at screening and Ravulizumab PK samples should be obtained in all patients taking Ravulizumab at screening.
²⁹For patients who receive Pozelimab IV infusion: obtain blood samples where permitted, prior to IV administration of Pozelimab and also within 15 minutes after the end of the IV infusion.
³⁰Blood samples for concentrations of Cemdisiran and its metabolites will be collected, where permitted, prior to any study treatment administration (pre-dose) and at 1 to 4 hours post dose. The post dose sample may be collected at the clinic or by a visiting health care professional (if available).
³¹Blood samples for ADA will be collected, where permitted, before the administration of any study drug (pre-dose). In the event of suspected SAEs, such as anaphylaxis or hypersensitivity, additional blood samples may be collected at or near the onset of the event for PK, ADA, and other analyses.
³²Samples for the measurement of concentrations of Cemdisiran and its metabolites will be collected at this visit only for patients who are receiving Eculizumab treatment and willing to continue in the transition period and into the OLE
³³Pozelimab and Cemdisiran ADA samples will also be collected pre-dose at this visit for patients who are received Eculizumab or Ravulizumab treatment and willing to continue in the transition period and into the OLE.
³⁴All biomarkers collected specifically on V3/day 1 must be collected pre-dose.
³⁵Whole blood samples for DNA extraction (optional) should be collected on day 1 (pre-dose) but can be collected at a later study visit.
³⁶For patients on Ravulizumab, visit 2r procedures should be completed on the day of or up to 2 days prior to Ravulizumab dose administration. Day 1 must be scheduled 4 weeks after the Ravulizumab dose or within 26 to 28 days counting from the day of the Ravulizumab dose.
³⁷Study treatment (Pozelimab/Cemdisiran, Eculizumab, or Ravulizumab) at week 36 is only for patients who intend to continue in the OLE. Patients who decline enrollment into OLE will not he dosed at week 36.

TABLE 6-2
Schedule of Events for the Transition Period: Patients on Eculizumab in OLTP who Plan to Enroll in the OLE

| | | Study Procedure (Visit) ¹ | |
|---|---|---|---|
| | | Transition period for patients on Eculizumab in OLTP | |
| | | (only for patients who complete OLTP and plan to | |
| | | enroll in OLE) | |
| | | Visit # | |
| | | (2 weeks after last dose of | |
| | | Eculizumab at OLTP week 36) | |
| | | TV1e² | TV2e |
| Weeks (after last dose of | | 2t | 4t |
| study treatment at OLTP week 36) | | | |
| Day | | 15t | 29t |
| Window (day) | | ±2 | ±2 |
| Baseline: | | | |
| Patient safety card for Neisseria meningitidis³ | | X | X |
| Treatment: | | | |
| IVRS/IWRS | | X | X |
| Anti-C5 | Administer Pozelimab 60 mg/kg IV⁵ | | X |
| standard-of-care | Administer Cemdisiran 200 mg SC⁵ | | X |
| arm: | Administer Pozelimab 400 mg SC⁵ | | X |
| Eculizumab⁴ | Administer Eculizumab 900 mg IV Q2W⁵ | X | |
| Concomitant meds and treatment | | X | X |
| Transfusion record update | | X | X |
| Antibiotics prophylaxis (recommended)⁷ | | X | X |
| Safety and Anthropometric: | | | |
| Body Weight | | X | X |
| Vital signs | | X | X |
| Adverse events | | X | X |
| Breakthrough hemolysis assessment⁸ | | X | X |
| Laboratory Testing⁹: | | | |
| Hematology¹⁰ | | X | X |
| Coagulation panel | | X | X |
| Blood chemistry (long panel) including LDH¹¹ | | X | X |
| Pregnancy test (applicable patients)¹² | | | X |
| Pharmacokinetics and Total C5 Sampling: | | | |
| Patients | Blood samples for conc. of Pozelimab¹³ | | X |
| previously | | | |
| taking | Blood samples for conc. of Eculizumab | | X |
| Eculizumab | Total C5 sample¹⁴ | | X |

| | | |
|---|---|---|
| CH50 | X | X |

Footnotes
¹ When multiple procedures are performed on the same day, the sequence of procedures is as follows: vital signs, blood collection (first coagulation draw then chemistry draw followed by all other labs) and study drug administration. It is particularly important that the scheduled blood draws are obtained prior to the administration of study treatment, especially efficacy parameters such as LDH (i.e., measurements reflect a time point at the end of the dosing interval).
²Transition period day 1 is the day of the week 36 Eculizumab dose given in the OLTP. During the transition period, Eculizumab may be administered within 2 days of all visits where Eculizumab administration applies. If Eculizumab administration does not coincide with the day of clinic visit, as applicable, then the clinic visit should always precede the infusion of Eculizumab.
³Patient safety card: provide the patient safety card for Neisseria meningitidis infection to the patient at any visit when needed. Site should review the instructions on the safety card with the patient at each visit.
⁴Patients randomized to the anti-C5 standard-of-care arm taking Eculizumab, who complete the 36-week OLTP and plan to enroll into the OLE study, will transition from Eculizumab to the combination treatment as follows: the patients will have received Cemdisiran 200 mg along with Eculizumab 900 mg IV on the week 36 End of Treatment Visit for the OLTP. Final Eculizumab 900 mg IV is on week 2t visit of the transition period, then Pozelimab 60 mg/kg IV along with Pozelimab 400 mg SC and Cemdisiran 200 mg SC on week 4t visit. Patients will transition to the OLE study after completing the week 4t visit.
⁵All study treatments (i.e., Pozelimab, Cemdisiran, and Eculizumab) should be administered as the last procedure after all blood sample collection and study assessments have been completed. A 2-hour observation period should be instituted following the administration of Cemdisiran on transition day 1 (week 36 of OLTP). On day 29 (week 4 of transition period), after completion of administration of Pozelimab 60 mg/kg IV, patients should be monitored for at least 30 minutes prior to administration of Pozelimab/Cemdisiran SC. A 2-hour observation period should be instituted after the last administration of study drug (whether Pozelimab or Cemdisiran SC).
6. Intentionally left blank
⁷Daily oral antibiotic prophylaxis against Neisseria meningitidis is recommended starting on the first day of dosing with study treatment and continuing until up to 52 weeks after discontinuation of Pozelimab/Cemdisiran. For post-treatment prophylaxis for Eculizumab or Ravulizumab follow the local prescribing information/national guidelines/local practice.
⁸If a patient is suspected of having a breakthrough hemolysis event, then in addition to the required laboratory collection, additional samples for CBC, coagulation parameters (including D-dimer), chemistry, reticulocyte count, total C5, CH50 and drug concentrations of Pozelimab, Cemdisiran, Eculizumab or Ravulizumab, ADA (against Pozelimab), and exploratory research serum and plasma will be collected unless already noted in the schedule of events for that visit. If the suspected event does not occur at a scheduled visit then an unscheduled visit should occur to evaluate the patient and to collect CBC, coagulation parameters (including D-dimer), chemistry, reticulocyte count, total C5, CH50 and drug concentrations of Eculizumab or Ravulizumab/Pozelimab/Cemdisiran, and ADA (against Pozelimab) as applicable, and exploratory research serum and plasma.
⁹During lab collection, handling and processing, the same methodology will be applied across study visits, as best as possible, to preserve the quality of sample and avoid hemolysis during sample processing. If the investigator or sponsor suspects that the lab result is not an accurate reflection of the patient's condition, consideration should be given to repeating the lab sample if clinically warranted and, in all cases, where an LDH is ≥2 × ULN in association with potassium ≥6 mmol/L. Blood collection should always be obtained prior to study treatment administration, unless otherwise noted.
¹⁰Hemoglobin will be assessed as part of the hematology analysis. Hematology sample should be collected before study treatment administration.
¹¹Serum LDH, CRP, and bilirubin will be assessed as part of the blood chemistry analysis. Obtain chemistry including LDH prior to any study treatment administration.
¹²Pregnancy test: A urine test will be done.
¹³For patients who receive Pozelimab IV infusion: obtain blood samples prior to IV administration of Pozelimab and also within 15 minutes after the end of the IV infusion.
¹⁴Blood sample for CH50 and total C5 will be collected pre-dose.

TABLE 6-3
Schedule of Events for the Transition Period: Patients
on Ravulizumab in OLTP who Plan to Enroll in the OLE

| | | Study Procedure (Visit) ¹ | |
|---|---|---|---|
| | | Transition period for patients on Ravulizumab in | |
| | | OLTP | |
| | | (only for patients who complete OLTP and plan to | |
| | | enroll in OLE) | |
| | | Visit # | |
| | | (4 weeks after last dose | |
| | | of Ravulizumab | |
| | | at OLTP week 36) | |
| | | TV1r² | TV2r |
| Weeks (after last dose of study treatment at OLTP | | 4t | 8t |
| week 36) | | | |
| Day | | 29t | 57t |
| Window (day) | | ±2 | ±2 |
| Baseline: | | | |
| Patient safety card for Neisseria meningitidis³ | | X | X |
| Treatment: | | | |
| IVRS/IWRS | | X | X |
| Anti-C5 | Administer Cemdisiran 200 mg SC⁵ | X | X |
| standard-of-care | | | |
| arm: | Administer Pozelimab 60 mg/kg IV⁵ | | X |
| Ravulizumab⁶ | Administer Pozelimab 400 mg SC⁵ | | X |
| Concomitant meds and treatment | | X | X |
| Transfusion record update | | X | X |
| Antibiotics prophylaxis (recommended)⁷ | | X | X |
| Safety and Anthropometric: | | | |
| Body Weight | | X | X |
| Vital signs | | X | X |
| Adverse events | | X | X |
| Breakthrough hemolysis assessment⁸ | | X | X |
| Laboratory Testing⁹: | | | |
| Hematology¹⁰ | | X | |
| Coagulation panel | | X | X |
| Blood chemistry (long panel) including LDH¹¹ | | X | X |
| Pregnancy test (applicable patients)¹² | | X | X |
| Pharmacokinetics and Total C5 Sampling: | | | |
| Patients | Blood samples for conc. of Pozelimab¹³ | | X |
| previously | Blood samples for conc. of Ravulizumab | | |
| taking | Total C5 sample¹⁴ | | X |
| Ravulizumab | | | X |

| | | |
|---|---|---|
| CH50 | X | X¹⁴ |

Footnotes
¹ When multiple procedures are performed on the same day, the sequence of procedures is as follows: vital signs, blood collection (first coagulation draw then chemistry draw followed by all other labs) and study drug administration. It is particularly important that the scheduled blood draws are obtained prior to the administration of study treatment, especially efficacy parameters such as LDH (i.e., measurements reflect a time point at the end of the dosing interval).
²Transition period day 1 is the day of the week 36 Ravulizumab dose given in the OLTP.
³Patient safety card: provide the patient safety card for Neisseria meningitidis infection to the patient at any visit when needed. Site should review the instructions on the safety card with the patient at each visit.
4. Intentionally left blank
⁵All study treatments (i.e., Pozelimab and Cemdisiran) should be administered as the last procedure after all blood sample collection and study assessments have been completed. A 2-hour observation period should be instituted following the administration of Cemdisiran on transition day 29 (week 4 of transition period). On day 57 (week 8 of transition period), after completion of administration of Pozelimab 60 mg/kg IV, patients should be monitored for at least 30 minutes prior to administration of the Pozelimab/Cemdisiran SC. A 2-hour observation period should be instituted after the last administration of study drug (whether Pozelimab or Cemdisiran SC).
⁶Patients randomized to the anti-C5 standard-of-care arm taking Ravulizumab, who complete the 36-week OLTP and plan to enroll into the OLE study will transition from Ravulizumab to the combination treatment as follows: the patients will have received the last dose of Ravulizumab at the week 36 End of Treatment Visit for the OLTP. They will enter into the transition period with the first visit at week 4t, 4 weeks after the last dose of Ravulizumab. At the week 4t visit, they will receive Cemdisiran 200 mg SC. At the week 8t visit they will receive Pozelimab 60 mg/kg IV along with Pozelimab 400 mg SC and Cemdisiran 200 mg SC. They will transition to the OLE study after completing the week 8t visit.
⁷Daily oral antibiotic prophylaxis against Neisseria meningitidis is recommended starting on the first day of dosing with study treatment and continuing until up to 52 weeks after discontinuation of Pozelimab/Cemdisiran. For post-treatment prophylaxis for Eculizumab or Ravulizumab follow the local prescribing information/national guidelines/local practice.
⁸If a patient is suspected of having a breakthrough hemolysis event, then in addition to the required laboratory collection, additional samples for CBC, coagulation parameters (including D-dimer), chemistry, reticulocyte count, total C5, CH50 and drug concentrations of Pozelimab, Cemdisiran, Eculizumab or Ravulizumab, ADA (against Pozelimab), and exploratory research serum and plasma will be collected unless already noted in the schedule of events for that visit. If the suspected event does not occur at a scheduled visit then an unscheduled visit should occur to evaluate the patient and to collect CBC, coagulation parameters (including D-dimer), chemistry, reticulocyte count, total C5, CH50 and drug concentrations of Eculizumab or Ravulizumab/Pozelimab/Cemdisiran, and ADA (against Pozelimab) as applicable, and exploratory research serum and plasma.
⁹During lab collection, handling and processing, the same methodology will be applied across study visits, as best as possible, to preserve the quality of sample and avoid hemolysis during sample processing. If the investigator or sponsor suspects that the lab result is not an accurate reflection of the patient's condition, consideration should be given to repeating the lab sample if clinically warranted and in all cases where an LDH is ≥2 × ULN in association with potassium ≥6 mmol/L. Blood collection should always be obtained prior to study treatment administration, unless otherwise noted.
¹⁰Hemoglobin will be assessed as part of the hematology analysis. Hematology sample should be collected before study treatment administration.
¹¹Serum LDH, CRP, and bilirubin will be assessed as part of the blood chemistry analysis. Obtain chemistry including LDH prior to any study treatment administration.
¹²Pregnancy test: A urine test will be done.
¹³For patients who receive Pozelimab IV infusion: obtain blood samples prior to IV administration of Pozelimab and also within 15 minutes after the end of the IV infusion.
¹⁴Blood sample for CH50 and total C5 will be collected pre-dose.

TABLE 6-4
Schedule of Events for the Safety Off-Treatment Follow-up Period

| | Visit | | | | | | |
|---|---|---|---|---|---|---|---|
| | | | | | | Phone | Phone |
| | | | | | | visit | visit |
| | FU-1 | FU-2 | FU-3 | FU-4 | FU-5 | FU-6 | FU-7 |
| | Weeks (after the last dose of study drug) | | | | | | |
| | 4 | 8 | 12 | 16 | 26 | 38 | 52 |
| | Window (day) | | | | | | |
| | ±10 | ±10 | ±10 | ±10 | ±10 | ±10 | ±10 |
| Body weight | X | X | X | X | X | | |
| Vital signs | X | X | X | X | X | | |
| Physical examination | | | X | | X | | |
| Concomitant meds/treatment | X | X | X | X | X | X | X |
| Adverse event reporting | X | X | X | X | X | X | X |
| Pregnancy reporting | X | X | X | X | X | X | X |
| Antibiotics prophylaxis (recommended)¹ | X | X | X | X | X | X | X |
| Patient safety card for Neisseria meningitidis² | X | X | X | X | X | X | X |

Laboratory Testing

| | | | | | |
|---|---|---|---|---|---|
| Hematology | X | X | X | X | X |
| Blood chemistry | X | X | X | X | X |

| | |
|---|---|
| Monthly urine pregnancy test (WOCBP)³ | <------------------------------------X--------------------------------> |

Pharmacokinetics, and ADA Sampling:

| | |
|---|---|
| Pozelimab PK sample | X |
| ADA sample for Pozelimab | X |
| ADA sample for Cemdisiran | X |
| Total C5 (plasma) | X |

Footnotes
¹Antibiotics prophylaxis (recommended): Daily oral antibiotic prophylaxis against Neisseria meningitidis is recommended starting at FU-1 and continuing until up to 52 weeks after discontinuation of Pozelimab/Cemdisiran. For post-treatment prophylaxis for Eculizumab or Ravulizumab follow the local prescribing information/national guidelines/local practice.
²Patient safety card for Neisseria meningitidis: Patient safety card: provide the patient safety card for Neisseria meningitidis infection to the patient at FU-1 or any other visit when needed. Site should review the instructions on the safety card with the patient at each visit.
³Pregnancy testing: Monthly urine pregnancy testing will be conducted for WOCBP only. If performed via at-home testing kits, patients should be reminded to call the study staff each month with the results of their pregnancy test, and as soon as possible if their pregnancy test result is positive.

### Inclusion Criteria

A patient must meet the following criteria to be eligible for inclusion in the study:

1. Male or female ≥18 years of age or legal age of majority, whichever is greater, at the time of consent
2. Diagnosis of PNH confirmed by a history of high-sensitivity flow cytometry from prior testing
3. (i) Ongoing treatment with Eculizumab* 900 mg IV Q 14 days for at least 12 weeks prior to screening visit.
or
(ii) Ongoing treatment with Ravulizumab* IV Q8W based on BW as follows, 3000 mg for BW≥40 kg to <60 kg, 3300 mg for BW≥60 kg to <100, 3600 mg for BW 100 kg for at least 24 weeks prior to screening visit.
   Note: Patients opting to participate from the R3918-PNH-2021 trial (A Randomized, Open-Label, Ravulizumab-Controlled Study to Evaluate the Efficacy and Safety of Pozelimab and Cemdisiran Combination Therapy in Patients with Paroxysmal Nocturnal Hemoglobinuria who are Complement Inhibitor Treatment-Naive or Have Not Recently Received Complement Inhibitor Therapy) who were randomized to the Ravulizumab arm must complete the open-label treatment period to be considered for eligibility in this study. *Biosimilars are not permitted, unless approved by the Sponsor.
   4. Provide informed consent signed by study patient.
   5. Willing and able to comply with clinic/remote visits and study-related procedures.
   6. Able to understand study-related questionnaires.

### Exclusion Criteria

Note: If a patient screen fails, and if the study is still ongoing, they may be rescreened (up to 2 times) if the Principal Investigator determines the patient may be eligible upon rescreening.

A patient who meets any of the following criteria will be excluded from the study:

1. Patients with a screening LDH>1.5×ULN who have not taken their C5 inhibitor within the labeled dose interval at the dose prior to the screening LDH assessment.
2. Receipt of an organ transplant, history of bone marrow transplantation or other hematologic transplant.
3. Body weight <40 kilograms at screening visit.
4. Current plans for modification (initiation, discontinuation, or dose/dosing interval change) of the following background concomitant medications, as applicable, during screening and treatment period: erythropoietin, immunosuppressive drugs, corticosteroids, anti-thrombotic agents, anticoagulants, iron supplements, and folic acid.
5. Any use of complement inhibitor therapy other than Eculizumab or Ravulizumab in the 26 weeks prior to the screening visit or planned use during the study with the exception of study treatments.
6. Any of the following abnormalities at the screening visit (two repeat measurements are allowed per parameter during screening period):
a. Peripheral blood absolute neutrophil count (ANC)<500/μL (<0.5×10⁹/L) or
b. Peripheral blood platelet count <30,000/μL or
c. Peripheral blood reticulocyte count abnormality defined as <60,000/μL (<0.06×10⁶/μL, <60×10⁹/L)
   Note: a patient will not be excluded if upon repeat testing the parameter no longer meets the exclusion criterion.
   Note: Patients receiving acute treatment (e.g., platelet transfusions, granulocyte colony stimulating factors) for these conditions during screening and in the 1-month preceding screening will not be eligible.
   7. Not meeting meningococcal vaccination requirements for Eculizumab or Ravulizumab according to the current local prescribing information (where available) and at a minimum documentation of meningococcal vaccination within 5 years prior to screening visit.
   Note: Patients without prior vaccination will be eligible provided they are willing to undergo vaccination prior to initiation of study treatment and vaccination is documented prior to randomization.
   8. Any contraindication for receiving Neisseria meningitidis vaccination.
   9. Unable to take antibiotics for meningococcal prophylaxis (if required by local Eculizumab or Ravulizumab prescribing information, where applicable or national guidelines/local practice or if vaccination is less than 2 weeks from study treatment initiation).
   10. Any active, ongoing infection or a recent infection requiring ongoing systemic treatment with antibiotics, antivirals, or antifungals within 2 weeks prior to screening or during the screening period.
   11. Documented history of systemic fungal disease or unresolved tuberculosis, or evidence of active or latent tuberculosis infection (LTBI) (i.e., if not having completed treatment for LTBI) during screening period. Assessment for active TB and LTBI should accord with local practice or guidelines, including those pertaining to risk assessment, and the use of tuberculin skin test or T-cell interferon gamma release assay.
   12. Positive hepatitis B surface antigen or hepatitis C virus RNA during screening.
   Note: Cases with unclear interpretation should be discussed with the medical monitor.
   13. Patients with known HIV with history of opportunistic infections in the last 1 year, any history of HIV related malignancy, documented history of CD4 count <500 cells/μL or detectable viral load within the last 6 months (Note: CD4 count and viral load must be available within the last 6 months, and may be conducted by a local laboratory during screening if needed).
   Note: Local testing for HIV may be conducted in patients if required locally or by local regulations.
   14. Documented* history of positive RT-PCR, antigen or serology test, or other health authority authorized test for SARS-CoV-2 and:
a. Have not recovered from COVID-19 (all COVID-19-related symptoms and major clinical findings which can potentially affect the safety of the patient should be resolved to baseline), and
b. Did not have 2 negative results from a health authority-authorized nucleic acid amplification (RT-PCR) test or other health authority authorized test for COVID 19 taken at least 48 hours apart prior to day 1.
   *Note: Screening for COVID-19 will not be performed as part of eligibility assessments for this study
   15. Known hereditary complement deficiency
   16. Documented history of active, uncontrolled, ongoing systemic autoimmune diseases
   17. Documented history of liver cirrhosis or patients with liver disease with evidence of current impaired liver function or patients with ALT or AST (unrelated to PNH or its complications) greater than 3×ULN at the screening visit (if the AST or ALT returns >3×ULN, one repeat assessment of the abnormal parameter(s) is allowed during screening).
   18. Patients with an eGFR of <30 mL/min/1.73 m² (according to Chronic Kidney Disease-Epidemiology Collaboration equation 2009) at screening visit (one repeat assessment allowed during screening).
   19. Recent, unstable medical conditions, excluding PNH and PNH related complications, within the past 3 months prior to screening visit (e.g., myocardial infarction, congestive heart failure with New York Heart Association Class≥III or IV, serious uncontrolled cardiac arrhythmia, cerebrovascular accident, active gastrointestinal bleed)
   20. Anticipated need for major surgery during the study
   21. History of cancer within the past 5 years, except for adequately treated basal cell skin cancer, squamous cell skin cancer, or in situ cervical cancer
   22. Participation in another interventional clinical study (except R3918-PNH-2021) or use of any experimental therapy within 30 days before screening visit or within 5 half-lives of that investigational product, whichever is greater, with the exception of Eculizumab or Ravulizumab.
   23. Known hypersensitivity to Eculizumab or Ravulizumab (as applicable), Pozelimab, Cemdisiran or to any components of their respective formulations.
   24. Patients with functional or anatomic asplenia
   25. Any clinically significant abnormality identified at the time of screening that in the judgment of the Investigator or sub-Investigator(s) would preclude safe completion of the study or constrain endpoints assessment such as major systemic diseases, or patients with short life expectancy
   26. Considered by the Investigator or sub-Investigator(s) as inappropriate for this study for any reason, e.g.,
a. Deemed unable to meet specific protocol requirements, such as scheduled visits.
b. Deemed unable to administer or tolerate need for chronic injections
c. Presence of any other conditions (e.g., geographic, social etc.) actual or anticipated, that the Investigator feels would restrict or limit the patient's participation for the duration of the study.
d. Part of a vulnerable population such as the institutionalized (this may also include patients who are committed to an institution by order issued either by the judicial or the administrative authorities, as applicable)
e. Patient ineligible for clinical trial participation due to local regulations (e.g., under legal protection measures [such as L1121-8 or L1121-8-1 in France], etc).
   27. Members of the clinical site study team and/or his/her immediate family, unless prior approval granted by the Sponsor.
   28. Pregnant or breastfeeding women.
   29. Women of childbearing potential (WOCBP)* who are unwilling to practice highly effective contraception prior to the initial dose/start of the first study treatment, during the study, and for at least 52 weeks after the last dose. Highly effective contraceptive measures include:
a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
b. intrauterine device (IUD); intrauterine hormone-releasing system (IUS);
c. bilateral tubal ligation or tubal occlusion;
d. vasectomized partner (provided that the male vasectomized partner is the sole sexual partner of the WOCBP study participant and that the vasectomized partner has obtained medical assessment of surgical success for the procedure); and/or
e. sexual abstinence†, ‡.
   *WOCBP are defined as women who are fertile following menarche until becoming postmenopausal, unless permanently sterile. Permanent sterilization methods include hysterectomy, bilateral salpingectomy, and bilateral oophorectomy.
   A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance.
   Pregnancy testing and contraception are required for WOCBP. Pregnancy testing and contraception are not required for women who are post-menopausal or permanently sterile.
   †Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the subject.
   ‡Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.
   30. Non-responsive to Eculizumab or Ravulizumab treatment (Note: non-responsive refers to patients with no reduction in LDH after receiving treatment with Eculizumab or Ravulizumab; patients with a response to Eculizumab or Ravulizumab that is sub-optimal are not excluded provided all other eligibility criteria are satisfied)
   31. Hemoglobin ≤7 g/dL (Note: A patient may receive a blood transfusion during the screening period and is eligible if repeat hemoglobin returns >7 g/dL prior to randomization. More than 2 repeat measurements are allowed.)

### Primary Objective

The primary objective of the study is to evaluate the effect of Pozelimab and Cemdisiran combination therapy on hemolysis, as assessed by LDH, after 36 weeks of treatment, in patients with PNH who switch from Eculizumab or Ravulizumab therapy to the combination treatment versus patients who continue their Eculizumab or Ravulizumab therapy.

### Secondary Objectives

The secondary objectives of the study are to:
Evaluate the effect of Pozelimab and Cemdisiran combination treatment versus anti C5 standard-of-care treatment (Eculizumab or Ravulizumab) on the following:
   Transfusion requirements and transfusion parameters
   Measures of hemolysis: LDH control, breakthrough hemolysis, and inhibition of CH50
   Hemoglobin levels
   Fatigue as assessed by Clinical Outcome Assessments (COAs)
   HRQoL as assessed by COAs
   Safety and tolerability
To assess the concentrations of total Pozelimab and either total Eculizumab or total Ravulizumab in serum and Cemdisiran and total C5 protein in plasma
To assess the immunogenicity of Pozelimab and Cemdisiran

### Exploratory Objectives

The exploratory objectives of the study are:
To explore the effect on clinical thrombosis events
To explore the effect on renal function and renal injury biomarkers
To explore the effect on complement activation and hemolysis relevant to PNH and other related diseases
To explore the effect on PNH clone size
To explore the effect on COAs measuring treatment satisfaction questionnaire for medication (TSQM), QoL (European Quality of Life-Five Dimension-Five Level Scale [EQ-5D-5L] and European organization for research and treatment of cancer quality of-Life questionnaire core 30 items [EORTC-QLQ-C30]), and PNH-specific symptoms
Develop a molecular understanding of PNH and related diseases, and study mechanism of action (including relationship to safety and efficacy) and complement pathway biology.
To explore whether potential differences in patient efficacy and safety are associated with genotype and gene expression and to further study C5, PNH, or other conditions associated with complement-mediated injury using optional whole blood DNA and RNA collection from consented patients.

### Primary Endpoint

The primary endpoint is the percent change in LDH from baseline to EOT period at week 36 (day 253).

### Key Secondary Endpoints

The key secondary endpoints are:
Transfusion avoidance after day 1 through week 36, inclusive (defined as not requiring an RBC transfusion as per protocol algorithm based on hemoglobin values after day 1)
Breakthrough hemolysis, in patients with a baseline LDH≤1.5×ULN, after day 1 through week 36, inclusive
Hemoglobin stabilization (defined as patients who do not receive an RBC transfusion and have no decrease in hemoglobin level from baseline of ≥2 g/dL) after day 1 through week 36, inclusive
Maintenance of adequate control of hemolysis, defined as LDH≤1.5×ULN from week 8 through week 36, inclusive
Adequate control of hemolysis (defined as LDH≤1.5×ULN) from week 8 through week 36, inclusive
Normalization of LDH, defined as LDH≤1.0×ULN from week 8 through week 36, inclusive
Change in fatigue as measured by the FACIT-Fatigue Scale from baseline to week 36
Change in PF score on the EORTC-QLQ-C30 from baseline to week 36
Change in global health status (GHS)/QoL scale score on the EORTC-QLQ-C30 from baseline to week 36

### Other Secondary Endpoints

Other secondary endpoints are:
Transfusion avoidance from week 4 through week 36, inclusive (defined as not requiring an RBC transfusion as per protocol algorithm based on hemoglobin values after day 1)
Breakthrough hemolysis, in patients with a baseline LDH≤1.5×ULN, from week 4 through week 36, inclusive
Hemoglobin stabilization (defined as patients who do not receive an RBC transfusion and have no decrease in hemoglobin level from baseline of ≥2 g/dL) from week 4 through week 36, inclusive
Maintenance of adequate control of hemolysis, defined as LDH≤1.5×ULN after day 1 through week 36, inclusive
Adequate control of hemolysis (defined as LDH≤1.5×ULN) after day 1 through week 36, inclusive
Normalization of LDH (defined as LDH≤1.0×ULN) after day 1 through week 36, inclusive
Rate and number of units of RBCs transfused per protocol algorithm after day 1 through week 36, and from week 4 through week 36
Change in hemoglobin levels from baseline to week 36
Incidence and severity of treatment-emergent SAEs, TEAEs of special interest and TEAEs leading to treatment discontinuation over 36 weeks
Change and percent change in total CH50 from baseline to week 36
Concentration of total C5 in plasma assessed throughout the study
Concentrations of total Pozelimab in serum assessed throughout the study
Concentrations of Cemdisiran in plasma assessed throughout the study
Concentrations of total Eculizumab or Ravulizumab in serum assessed throughout the study
Incidence of treatment emergent anti-drug antibodies (ADAs) to Pozelimab assessed throughout the study
Incidence of treatment emergent ADAs to Cemdisiran assessed over 36 weeks

### Exploratory Endpoints

The exploratory endpoints are:
Incidence of major adverse vascular events (MAVE) from day 1 through week 36, and from week 4 through week 36
Breakthrough hemolysis (rate) after day 1 to week 36 in patients with a baseline LDH of <1.5×ULN
Change in renal function as measured by estimated glomerular filtration rate (eGFR) from pre-treatment day 1 to week 36
Percent change in free hemoglobin from baseline to week 36
Change in haptoglobin from baseline to week 36
Change in total bilirubin from baseline to week 36
Change in reticulocyte count from baseline to week 36
Change and percent change in alternative pathway hemolytic activity assay (AH50) from baseline to week 36
Change in PNH erythrocytes, granulocytes, and monocytes from baseline to week 36
Change in treatment satisfaction as assessed by the TSQM from baseline to week 36
Change in EQ-5D-5L scores from baseline to week 36
Change in PNH symptoms as measured by the PNH symptom-specific questionnaire scores from baseline to week 36
Change from baseline to week 36 in functional scale scores (Role Functioning, Emotional Functioning, Cognitive Functioning and Social Functioning) and symptom scale scores (Fatigue, Nausea and Vomiting, Pain, Dyspnea, Insomnia, Appetite Loss, Constipation, Diarrhea) of the EORTC-QLQ-C30
Stability in global health status, functioning and symptoms as measured by the EORTC-QLQ-C30 from baseline to week 36 Efficacy Variables-Laboratory Variables for the Assessment of Efficacy
   Efficacy in this Study is Evaluated by the Following Laboratory Assessments:
LDH (serum): LDH as a measure of intravascular hemolysis allows for an objective and precise means to gauge whether the control of intravascular hemolysis is sustained when the patients are switched to Pozelimab and Cemdisiran combination treatment
Hemoglobin: Hemolytic anemia is a hallmark of PNH

These laboratory variables are relevant to the characterization and disease mechanisms of PNH (Brodsky RA. Paroxysmal nocturnal hemoglobinuria. Blood 2014; 124(18):2804-2811.).

### Transfusion Record

Hemolytic anemia is a clinical manifestation of PNH, and patients often require blood transfusion for symptomatic management. The frequency of blood transfusion has been used in other studies of PNH to assess efficacy (Hillmen P, Muus P, Dührsen U, Risitano A M, Schubert J, Luzzatto L, et al. Effect of the complement inhibitor Eculizumab on thromboembolism in patients with paroxysmal nocturnal hemoglobinuria. Blood 2007; 110(12):4123-4128) (Röth A, Egyed M, Ichikawa S, Kim J S, Nagy Z, Gasl Weisinger J, et al. The SMART Anti-hC5 Antibody (SKY59/RO7112689) Shows Good Safety and Efficacy in Patients with Paroxysmal Nocturnal Hemoglobinuria (PNH). Blood 2018; 132(Suppl 1):535).

### Clinical Outcome Assessments

Brief descriptions of COAs are provided in Section 9.2.3 and include the following:
FACIT-Fatigue
EORTC-QLQ C30
TSQM
EQ-5D-5L
PNH Symptom-Specific Questionnaire

### Safety Variables and Anthropometric Variables

Safety and anthropometric variables in this study include:
Vaccination/revaccination for Neisseria meningitidis,
Vaccination against Streptococcus pneumoniae and Haemophilus influenzae type B (if needed)
Concomitant medications and procedures
Height
Body weight
Vital Signs
Physical Examination
Electrocardiogram (ECG)
AEs
Breakthrough hemolysis assessment
Routine safety laboratory tests (hematology, chemistry, coagulation parameters, direct antiglobulin test, urinalysis, and pregnancy testing [for women of childbearing potential or WOCBP])

### Example 7: A Randomized, Open-Label, Ravulizumab-Controlled, Non-Inferiority Study to Evaluate the Efficacy and Safety of Pozelimab and Cemdisiran Combination Therapy in Patients with Paroxysmal Nocturnal Hemoglobinuria Who are Complement Inhibitor Treatment-Naive or have not Recently Received Complement Inhibitor Therapy (R3918-PNH-2021)

This study is a randomized, open-label, Ravulizumab-controlled, non-inferiority study. The study is planned to enroll patients with PNH who are complement inhibitor treatment-naive or have not recently received complement inhibitor therapy.

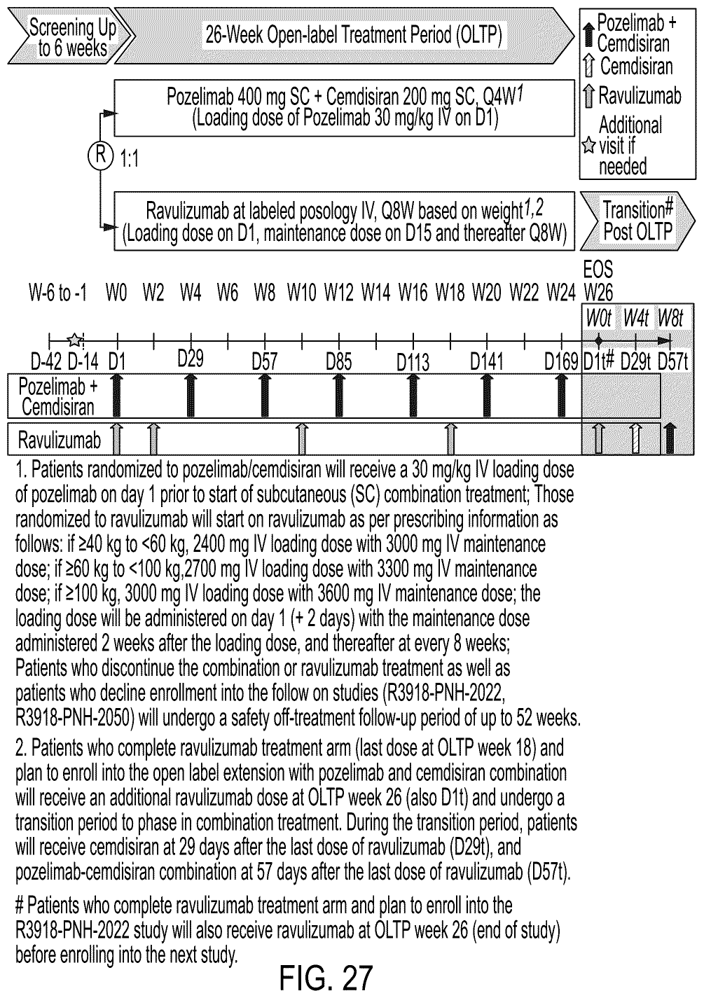

The study consists of the following periods: up to 6-week screening period and a 26-week OLTP with either Ravulizumab or the combination of Pozelimab and Cemdisiran (FIG. 27). Patients who complete the Ravulizumab OLTP and plan to be screened for the R3918-PNH-2022 study or enroll in the follow on open-label long-term extension study with Pozelimab and Cemdisiran combination will follow additional post-OLTP procedures. Patients who discontinue study treatment as well as patients who do not go on to participate in the R3918-PNH-2022 or decline enrollment into the OLE study will undergo a safety off-treatment follow-up period of up to 52 weeks.

### Screening Period

The screening period of up to 6 weeks prior to day 1 will evaluate patients to establish their eligibility to enter the study. Historical data related to prior and concomitant medications, hemolytic parameters, and RBC transfusions as well as PNH symptoms will be collected.

Additional interim screening visit(s) may take place as needed, for instance repeat blood collection.

Due to the risk of Neisseria meningitidis infection, patients will require administration of meningococcal vaccination(s) in accordance with local Ravulizumab prescribing information, where applicable, and in accordance with current national vaccination guidelines for vaccination use with complement inhibitors or local practice and at a minimum, within a period of 5 years prior to screening. For patients who require administration with meningococcal vaccination(s) during the screening period, administration should occur preferably at least 2 weeks prior to day 1, or at another time point according to local practice or national guidelines/local Ravulizumab prescribing information (where available). If vaccination precedes the initiation of study treatment by less than 2 weeks, then the patient must receive antibiotic prophylaxis for a minimum of 2 weeks from the date of vaccine administration.

Patients who have not been vaccinated against Streptococcus pneumoniae and Haemophilus influenzae type B may receive these vaccinations during the screening period or on the day of randomization based on investigator discretion and taking into consideration the available national guidelines.

In addition to Neisseria meningitidis infection, fatal or serious infections with Neisseria gonorrhea have been reported in patients taking complement inhibitors. Therefore, patients should undergo a risk assessment and counseling regarding the potential risk of Neisseria gonorrhea infection as per local practice or national guidelines.

Patients will be assessed for active or latent tuberculosis (TB) infection based on local practice or applicable guidelines. Based on the risk assessment, the need for screening with either tuberculin skin test or T-cell interferon-gamma release assay will be made. The interpretation of these results, as applicable, will be made by the investigator. Further management and treatment of TB will be the responsibility of the investigator.

Patients who complete the screening assessments and are deemed eligible for enrollment will be randomized in a 1:1 ratio into the combination arm or the Ravulizumab arm. Randomization will be stratified based on the factors described herein-Patients will be randomized by an Interactive Web Response System (IWRS) in a 1:1 ratio to receive either the combination treatment with SC Pozelimab 400 mg and Cemdisiran 200 mg Q4W or IV Ravulizumab according to the labeled weight-based dosing algorithm. Stratified, blocked randomization will be performed using the following stratification factors:
RBC/whole blood transfusion within the past 1 year prior to randomization (yes/no) Note: Enrollment of patients who have had no RBC transfusion in the past 1 year before randomization will be capped at 20% of the overall enrolled population.
Screening visit LDH levels (2.0 to <4.0×ULN or 24.0×ULN)

Randomization blocking will be performed centrally.

### Open-Label Treatment Period (OLTP)

The treatment period is 26 weeks (Table 7-1). Patients will receive the corresponding treatments according to their treatment assignment described below.
Ravulizumab Arm:
Patients who are randomized to the Ravulizumab arm will start Ravulizumab treatment on day 1 (+2 days) as per the prescribing information for Ravulizumab as follows:

| | |
|---|---|
| Day 1 (Ravulizumab may be given | Loading dose based on weight (≥40 kg to <60 |
| on the same day after visit | kg, 2400 mg IV; ≥60 kg to <100 kg, 2700 mg |
| procedures are completed or up to 2 | IV; ≥100 kg, 3000 mg IV) |
| days after the visit): | |
| Day 15 (As the first maintenance | Maintenance dose based on weight (≥40 kg |
| dose is to be administered 2 weeks | to <60 kg, 3000 mg IV; ≥60 kg to <100 kg, |
| after the loading dose, the week 2 | 3300 mg IV; ≥100 kg, 3600 mg IV) and |
| [day 15] visit should be scheduled on | thereafter every 8 weeks (Q8W) (±7 days) |
| the day of or up to 2 days preceding | |
| the Ravulizumab administration.) | |

Pozelimab and Cemdisiran Arm:
   Patients who are randomized to Pozelimab and Cemdisiran combination arm will receive the following treatments during the study:

| | |
|---|---|
| Day 1* | A single loading dose of Pozelimab 30 mg/kg |
| | IV followed by Pozelimab 400 mg SC and |
| | Cemdisiran 200 mg SC (the combination |
| | maintenance dose) |
| Day 29 | Combination maintenance dose (Pozelimab |
| | 400 mg and Cemdisiran 200 mg) SC and |
| | thereafter Q4W |

*Note:
On day 1, the time from the completion of the Pozelimab IV infusion to the start of the combination SC injections should be at least 30 minutes. Patients should be monitored for at least another 30 minutes after completing the first SC injections for Pozelimab and Cemdisiran. Note that Pozelimab and Cemdisiran should be administered at different anatomical sites on the day of the SC injections (e.g., left thigh, right thigh). Injection sites should be rotated for subsequent injections.
In addition, emergency equipment and medication for the treatment of infusion reactions must be available at the clinical site for immediate use. All infusion reactions must be reported as AEs and graded.

### Administration of Maintenance Regimen of Pozelimab and Cemdisiran

After day 1 dosing, subsequent administrations may be continued by the site personnel or a healthcare professional at the patient's home (if possible and approved by the sponsor) or for the combination of Pozelimab and Cemdisiran, administered by the patient or designated person at the patient's preferred location. These various options for administration will depend on preference of the investigator and patient, local regulations as well as availability of healthcare professional with sponsor endorsement. If self-administration/administration by designated person is undertaken for the combination of Pozelimab and Cemdisiran, then sufficient injection training at the scheduled administration(s) with Pozelimab and Cemdisiran maintenance regimen will be provided. After training, observation of self-administration/administration by designated person will be conducted by clinical site personnel or visiting healthcare professional/virtual visit (if available). Once this observation is considered satisfactory, the Pozelimab and Cemdisiran maintenance regimen can be subsequently administered independently by patient/designated person for the remainder of the study. Patients who self-administer/have the study drug administered by a designated person shall complete a patient diary to collect information on study treatment administration. They shall also be contacted by the study site to ensure study drug administration as planned.

### Post-Open-Label Treatment Period (Post-OLTP)

All patients who complete the 26-week OLTP study treatment will be offered to participate in the long-term, OLE study of the combination treatment.

Patients randomized to the Ravulizumab arm who complete the 26-week OLTP and plan to enroll into the next study will be given an additional Ravulizumab dose at week 26 of the OLTP. These patients will first be offered to participate in the R3918 PNH 2022 study (a Pozelimab and Cemdisiran combination study in patients with PNH who are currently treated with Eculizumab or Ravulizumab). If recruitment for the R3918-PNH-2022 is open, the patient will proceed with screening as described for the R3918-PNH-2022 study.

If the patient does not meet eligibility requirements for the R3918-PNH-2022 study, or if recruitment is closed for R3918-PNH-2022, the patient may proceed into the OLE study. The transition from Ravulizumab to Cemdisiran and Pozelimab should be managed in the following manner (Table 7-2):
Transition day 1 (week 26 of OLTP): Ravulizumab at the labeled weight-based dose
Transition day 29 (week 4 of transition period): Cemdisiran 200 mg SC
Transition day 57 (week 8 of transition period): Pozelimab 60 mg/kg IV loading dose followed by Pozelimab 400 mg SC and Cemdisiran 200 mg SC

This approach for patients randomized to Ravulizumab and who plan to enroll in the OLE study directly is to ensure a consistent dosing approach when switching from Ravulizumab to Pozelimab and Cemdisiran combination therapy.

For patients who are randomized to the Pozelimab and Cemdisiran combination arm, day 1 visit of the OLE study will occur 2 weeks after the week 26 end of study (EOS) visit in the current study to ensure that there is no interruption of study treatment.

Patients who discontinue study treatment as well as patients who do not go on to participate in the R3918-PNH-2022 or decline enrollment into the OLE study will undergo a safety off-treatment follow-up period of up to 52 weeks according to Table 7-3. Patients discontinuing study treatment should be treated in accordance with local standards of care while continuing to be monitored in the 52-week off-treatment safety follow-up period. Investigators switching patients from the combination treatment to another anti-C5 mAb should have a heightened awareness for possible AEs resulting from the formation of large DTD immune complexes.

The main study is considered finished when all patients either complete the 26-week treatment period or prematurely discontinue the study. Additional data collected during the transition period and safety off-treatment follow-up period will be described separately.

### Schedule of Events

In light of the public health emergency related to COVID-19, the continuity of clinical study conduct, and oversight may require implementation of temporary or alternative mechanisms. Examples of such mechanisms may include, but are not limited to, any of the following: phone contact, virtual visits, telemedicine visits, online meetings, non-invasive remote monitoring devices, use of local clinic or laboratory locations, and home visits by skilled staff. Additionally, no waivers to deviate from protocol enrollment criteria due to COVID-19 will be granted. All temporary mechanisms utilized, and deviations from planned study procedures are to be documented as being related to COVID-19 and will remain in effect only for the duration of the public health emergency.

Study assessments and procedures are presented by study period and visit in Table 7-1 (OTLP), Table 7-2 (Transition Period), and Table 7-3 (Safety Follow-up Period). See FIG. 27.

TABLE 7-1
Schedule of Events for the Open-Label Treatment Period

| Study Procedure (Visit)² | | | | | | | | | | | | |
|---|---|---|---|---|---|---|---|---|---|---|---|---|
| | | Open-Label Treatment Period (OLTP)¹ | | | | | | | | | | |
| Screening | | | | | | | | | | | | EOS |
| V1 | V2 | V3 | V4 | V5 | V6 | V7 | V8 | V9 | V10 | V11 | V12 | V13 |
| Week | | | | | | | | | | | | |
| Up | −4 | | | | | | | | | | | |
| to −6 | to −2 | 0 | 2 | 4 | 8 | 10 | 12 | 16 | 18 | 20 | 24 | 26 |
| Day | | | | | | | | | | | | |
| Up | −28 | | | | | | | | | | | |
| to −42 | to −14 | 1 | 15 | 29 | 57 | 71 | 85 | 113 | 127 | 141 | 169 | 183 |

Window (day)

| | | | | | | | | | |
|---|---|---|---|---|---|---|---|---|---|
| ±2 | ±3 | ±3 | ±3 | ±3 | ±7 | ±3 | ±3 | ±7 | ±3 |

Screening/Baseline:

| eCOA device dispensation | | X | |
|---|---|---|---|
| Inclusion/Exclusion | X | X | X |
| Informed consent | X | | |
| FBR informed consent (optional) | X | | |
| Genomics informed consent (optional) | X | | |
| Medical history³ | | | |
| Prior medications⁴ | X | | |
| Demographics | X | | |
| Height | X | | |
| Hepatitis B and C testing | X | | |
| Vaccination/revaccination for Neisseria | <------- X ------> | | |
| meningitidis⁵ | | | |
| Vaccination against Streptococcus pneumoniae | <------- X ------> | | |
| and Haemophilus influenzae type B (if needed)⁶ | | | |
| Tuberculosis history and assessment⁷ | X | | |
| Risk assessment for Neisseria gonorrhea ⁸ | X | | |
| Randomization | | | |

Treatment:

| IVRS/IWRS | | X | X | X | X | X | X | X | X | X | X | X | X |
|---|---|---|---|---|---|---|---|---|---|---|---|---|---|
| Ravulizumab | Ravulizumab IV Q8W¹⁰ | | X | | | | X | | | X | | | X³⁰ |
| arm | | | | | | | | | | | | | |
| Pozelimab | Pozelimab 30 mg/kg IV loading | | X¹² | | | | | | | | | | |
| and | dose | | | | | | | | | | | | |
| Cemdisiran | Pozelimab 400 mg SC Q4W¹² | | X¹² | | X | X | | X | | | X | X³¹ | |
| treatment | Cemdisiran 200 mg SC Q4W¹² | | X | | X | X | | X | X | | X | X³¹ | |
| arm¹¹ | | | | | | | | | | | | | |

| | | | | | | | | | | | | | |
|---|---|---|---|---|---|---|---|---|---|---|---|---|---|
| Injection training/patient instructions¹² | | | | | X | | | X | | | X | | |
| Patient diary¹³ | | | | | | | X | | | | X | | |
| Concomitant meds and procedures | X | X | X | X | X | X | X | X | | X | X | X | X |
| Transfusion record update | X | X | X | X | Xrecent | X | X | X | X | X | X | X | X |

| | |
|---|---|
| Antibiotics prophylaxis (recommended)¹⁴ | <--------------------------------X---------------------------------> |

Clinical outcome assessments (COAs):

| FACIT-Fatigue | | X | X | X | X | X | X | X | X | X |
|---|---|---|---|---|---|---|---|---|---|---|
| EORTC-QLQ-C30 | | X | X | X | X | X | X | X | | X |
| EQ-5D-5L | | | | | | | | | | |
| TSQM | | X | | X | X | X | X | X | X | X |
| PNH symptom-specific questionnaire (daily)¹⁵ | <---------------------------------------X-------------------------------------> | | | | | | | | | |
| PGIS (PNH Symptoms/Impacts/Fatigue) | | X | X | X | X | X | X | X | X | X |
| PGIC (PNH Symptoms/Impacts/Fatigue) | | | | X | | X | | | X | X |

Safety and Anthropometric:

| | | | | | | | | | | | | | |
|---|---|---|---|---|---|---|---|---|---|---|---|---|---|
| For Ravulizumab arm only: Provide patient safety | | | X | | | | | | | | | | |
| brochure for Ravulizumab | | | | | | | | | | | | | |
| Patient safety card for Neisseria meningitidis ⁹ | | | | | X | X | X | X | X | X | X | X | X |
| Body weight | X | | X | | X | X | X | X | X | X | X | X | X |
| Vital signs¹⁶ | | | X | | | | X | X | X | X | X | X | |
| Physical examination¹⁷ | | | | | | | | | | | | | |
| Electrocardiogram | | | | | | | | | | | | | |
| Adverse events | X | X | X | X | X | X | X | X | X | X | X | X | X |
| Breakthrough hemolysis assessment¹⁸ | X | X | X | | X | X | X | X | X | X | X | X | X |

Laboratory Testing¹⁹:

| | | | | | | | | | | | |
|---|---|---|---|---|---|---|---|---|---|---|---|
| Titers to measure N. meningitidis (only if required | X | | | | | | | | | | |
| per local practice/regulations) | | | | | | | | | | | |
| Hematology²⁰ | | X | X | | | X | X | X | X | X | X |
| Coagulation parameters | > | | | | X | X | X | | X | | X |
| Blood chemistry (long panel) including LDH²¹ | X | X | X | | X | | X | X | X | | X |
| D-dimer | | | | | | X | | | | | X |
| Immunoglobulin G | | | X | | | X | | | | | |
| Pregnancy test (applicable patients): serum (S) or | S | U | U | U | | U | U | | U | U | U |
| urine (U) | | | | | | | | | | | |
| Urinalysis | X | X | X | X | | X | X | | X | X | X |
| Direct antiglobulin test (DAT or Coombs test) | | X | | | | | | | | | X |

Pharmacokinetics, ADA, and Total C5 Sampling:

| | | | | | | | | | | | |
|---|---|---|---|---|---|---|---|---|---|---|---|
| Pozelimab | Pozelimab PK sample²² | X | X | X | | X | X | | X | X | |
| and | Cemdisiran and its metabolites PK | | | | | X | | | X | | |
| Cemdisiran | sample²³ | | | | | | | | | | |
| arm | ADA sample for Pozelimab²⁵ | | | | | | | | | | |
| | ADA sample for Cemdisiran²⁵ | | | | | | | | | | |
| | Total C5 (plasma)²⁷ | X | X | X | | X | X | | X | X | X |
| Ravulizumab | Ravulizumab PK²⁴ | | | | X | X | X | X | | X | X |
| arm | ADA sample for Pozelimab²⁵ | | | | | | | | | | X²⁶ |
| | ADA sample for Cemdisiran²⁵ | | | | | | | | | | X²⁶ |
| | Total C5 (plasma)²⁷ | X | X | X | | X | X | | X | X | X |

Biomarkers:

| | | | | | | | | | | | |
|---|---|---|---|---|---|---|---|---|---|---|---|
| Free hemoglobin²⁷ | X | X²⁸ | | | | X | | | X | | X |
| Haptoglobin²⁷ | | X²⁸ | | X | X | X | | | X | | X |
| Complement hemolytic assay (serum CH50 and | | X²⁸ | X | X | X | X | X | X | X | X | X |
| AH50)²⁷ | | | | | | | | | | | |
| sC5b-9 (plasma)²⁷ | | X²⁸ | X | X | X | X | X | X | X | X | X |
| PNH erythrocyte cells²⁷ | X | | | | | X | | | | | X |
| PNH granulocytes and monocytes²⁷ | X | | | | | X | | | | | X |
| Exploratory research serum sample²⁷ | | X²⁸ | | | X | | | | | | X |
| Exploratory research plasma sample²⁷ | | X²⁸ | | | X | | | | X | | X |

Optional pharmacogenomics (RNA & DNA)

| | | | |
|---|---|---|---|
| Whole blood sample for DNA isolation | X | | |
| (optional)²⁹ | | | |
| Whole blood sample for RNA isolation (optional) | X | X | X |

Footnotes
¹Study procedure visits on days 15, 71, 113, 127, 141, and 169 may be at the clinical site, or another preferred location such as patient's home. The location will be dependent on availability (and if approved by the sponsor) of home healthcare visiting professional, and preferences of the investigator and patient. Visits may also be conducted at another preferred location depending on extenuating circumstances such as due to SARS-CoV-2 infection provided the assessments can be performed by the visiting healthcare professional.
²When multiple procedures are performed on the same day, the sequence of procedures is as follows: COA assessments; ECG/vital signs/physical examination; blood collection (first coagulation draw then chemistry draw followed by all other labs); and study drug administration. It is particularly important that the scheduled blood draws are obtained prior to the administration of Ravulizumab or Pozelimab and Cemdisiran, especially efficacy parameters such as LDH (i.e., measurements reflect a time point at the end of the dosing interval). During blood sample collection, handling, and processing, the same methodology will be applied across study visits, as best as possible, to preserve the quality of sample and avoid hemolysis during sample processing. If the investigator or sponsor suspects that the lab result is not an accurate reflection of the patient's condition, consideration should be given to repeating the lab sample if clinically warranted and, in all cases, where an LDH is ≥2 × ULN in association with potassium ≥6 mmol/L. Specific instructions for avoiding hemolysis are provided in the relevant section of the protocol.
³Medical history including, transfusions, breakthrough hemolysis history, and laboratory parameters for measurement of hemolysis (such as LDH, bilirubin, haptoglobin, reticulocyte count, and hemoglobin) should be obtained for the past 52 weeks, if possible. Prior history of thrombosis and infections of the Neisseria spp. will be collected. Patients with a known C5 mutation (i.e., C5 variants R885H/C) are not eligible, however, if a C5 mutation is later suspected and confirmed while the study is ongoing, the information should be included as part of the patient's medical history. Patients who are poor responders to Ravulizumab treatment during the study may be asked for a mutation analysis to be conducted as part of the study, if the patient agrees to such testing.
⁴Including detailed medication history for PNH treatment and Neisseria meningitidis vaccination history and other vaccinations as applicable.
⁵Patients will require administration with meningococcal vaccination unless documentation is provided of prior immunization in the past 5 years or less than 5 years if required according to current national vaccination guidelines for vaccination use with complement inhibitors/local Ravulizumab prescribing information. For patients who require administration with meningococcal vaccination(s) during the screening/period, administration should occur preferably at least 2 weeks prior to day 1, or at another time point according to local Ravulizumab prescribing information/national guidelines.
⁶Vaccination for Streptococcus pneumoniae and Haemophilus influenzae Type B should be per current national/local vaccination guidelines.
⁷Screening by tuberculin skin test or T-cell interferon-gamma release assay may be performed according to local practice or guidelines at the discretion of the investigator.
⁸A risk factor assessment for Neisseria gonorrhea will be performed in accordance with local practice/national guidelines, and regular testing and counseling is advised for at-risk patients.
⁹Patient safety card for Neisseria meningitidis infection will be provided to the patient on day 1 or any other visit when needed. Site should review the instructions on the safety card with the patient at each visit.
¹⁰Patients who are randomized to Ravulizumab will receive Ravulizumab according to the labeled posology with the first dose of Ravulizumab administered on day 1 (or up to 2 days after the day 1 visit) according to the patient's weight (>40 kg to <60 kg, 2400 mg IV; ≥60 kg to <100 kg, 2700 mg IV; ≥100 kg, 3000 mg IV). The first maintenance dose should be administered 2 weeks after the loading dose as follows: (>40 kg to <60 kg, 3000 mg IV; ≥60 kg to <100 kg, 3300 mg IV; ≥100 kg, 3600 mg IV). Thereafter, the maintenance dose should be administered IV Q8W (+7 days). As the first maintenance dose is to be administered 2 weeks after the loading dose, the week 2 visit should proceed within the visit window, the day of or preceding the Ravulizumab administration. If Ravulizumab administration does not coincide with the day of clinic visit, as applicable, assuming that both the treatment and visit windows are respected, then the clinic visit should always precede the infusion of Ravulizumab dosing. A body weight should be performed prior to administration of Ravulizumab to allow for weight-based dosing. Patients who opt not to enroll into the follow on OLE study or who will not screen for the R3918-PNH-2022 study will have the last dose of Ravulizumab at week 18 (day 127).
¹¹Patients who are randomized to Pozelimab and Cemdisiran combination arm will receive Pozelimab 30 mg/kg IV along with Pozelimab 400 mg SC and Cemdisiran 200 mg SC on day 1. Patients should be monitored for at least 30 minutes after completion of Pozelimab 30 mg/kg IV and should be monitored for at least another 30 minutes after the completion of the first Pozelimab and Cemdisiran SC dosing. Subsequent SC doses of the combination will be administered Q4W and may either be performed by the site personnel or another healthcare professional at patient's home (if available), or administration by patient or designated person at the patient's preferred location. The final dosing of the SC combination in the OLTP is at week 24. The dose of Pozelimab and Cemdisiran should be given Q4W (every 28 days) and on the day of the corresponding study visit whenever possible. Study treatment administration should always be the last procedure after all blood sample collection and study assessments have been completed. If Pozelimab or Cemdisiran cannot be administered on the day of the corresponding study visit, the combination may be administered up to 7 days before or up to 7 days after the planned dosing date as long as the dosing takes place after the corresponding study visit has been completed. For example, the week 8 (D57) visit can take place from D54 to D60 given the visit window. The dose of Pozelimab and Cemdisiran therefore can be given from D54 to D64 but only on or after the week 8 visit assessments have been performed. Similarly, the week 16 (D113) visit can take place from D106 to D 120 given the visit window. The dose of Pozelimab and Cemdisiran can be given from D106 to D120 but only on or after the week 16 visit assessments have been performed. Pozelimab and Cemdisiran should be administered on the same day whenever possible.
¹²If the sponsor has endorsed self-injection, injection training will be provided to patients who desire self-injection or injection by a designated person. Site should observe patient syringe preparation and self-injection or injection by a designated person and confirm adequacy. Patient instruction materials will be provided (or reviewed as needed).
¹³Patient diary: If needed, for self-administration or administration by a designated person with Pozelimab and Cemdisiran combination treatment only, a patient diary may be provided to collect information on study treatment administration. Patient diary may be provided at week 4 visit or a subsequent visit. If patient diary is provided to the patient, then it should be reviewed at each clinic visit and data collected into the case report forms (CRFs). On the final visit, the diary should be collected by the site.
¹⁴Daily oral antibiotic prophylaxis against Neisseria meningitidis is recommended starting on the first day of dosing with study treatment and continuing until up to 52 weeks after discontinuation of Pozelimab and Cemdisiran. For post-treatment prophylaxis for Ravulizumab, consult the local prescribing information/national guidelines/local practice. If vaccination for Neisseria meningitidis occurs less than 2 weeks prior to day 1, then antibiotic prophylaxis must be administered for at least 2 weeks from the day of vaccination.
¹⁵Patient will complete PNH Symptom-Specific Questionnaire daily for 14 days prior to day 1 visit and continuing through the OLTP.
¹⁶Vital signs include temperature, sitting blood pressure and pulse. Vital signs will be obtained pre-dose after the patient has been sitting quietly for at least approximately 5 minutes, where applicable.
¹⁷Physical examination will include an evaluation of the head and neck, lungs, heart, abdomen, extremities, and skin. Care should be taken to examine and assess any abnormalities that may be present, as indicated by the patient's medical history.
¹⁸If a patient is suspected of having a breakthrough hemolysis event, then in addition to the required laboratory collection, additional samples for CBC, coagulation parameters (including D-dimer), chemistry, reticulocyte count, total C5, CH50, drug concentrations of Pozelimab/Ravulizumab/Cemdisiran, ADA (against Pozelimab) and exploratory research serum and plasma will be collected unless already noted in the schedule of events for that visit. If the suspected event does not occur at a scheduled visit, then an unscheduled visit should occur to evaluate the patient and to collect CBC, coagulation parameters (including D-dimer), chemistry, reticulocyte count, total C5, CH50 drug concentrations of Ravulizumab/Pozelimab/Cemdisiran, and ADA (against Pozelimab). In addition, an exploratory research serum and plasma sample should be collected.
¹⁹Clinical lab samples will be collected first before study drug administration. The coagulation blood sample must always be collected first, followed immediately by the blood chemistry sample. The same methodology will be applied across study visits, as best as possible, to preserve the quality of sample and avoid hemolysis during sample processing.
²⁰Hemoglobin will be assessed as part of the hematology analysis. Hematology sample should be collected before study treatment administration.
²¹Serum LDH, CRP, and bilirubin will be assessed as part of the blood chemistry analysis. Blood chemistry sample should be collected before study treatment administration.
²²Blood samples for Pozelimab PK will be obtained on the specified days prior to the Pozelimab dosing. On study visit day 1, obtain blood samples prior to IV administration of Pozelimab and also within 15 minutes after the end of the Pozelimab IV infusion.
²³Cemdisiran and its metabolite PK samples will be collected on the specified days before and 1 to 4 hours post Cemdisiran administration. The post dose sample may be collected at the clinic or by a visiting health care professional (if available).
²⁴Blood samples for Ravulizumab PK will be obtained prior to IV administration of Ravulizumab and also within 15 minutes after the end of Ravulizumab IV infusion.
²⁵Blood samples for ADA will be collected on the specified days before the study drug administration. In the event of suspected SAEs, such as anaphylaxis or hypersensitivity, additional blood samples may be collected at or near the onset of the event for PK, ADA, and other analyses.
²⁶For patients randomized to combination arm as well as patients in Ravulizumab arm who will be continuing in the transition period, blood samples are collected pre-dose to assess ADA of Pozelimab and Cemdisiran.
²⁷Blood samples for total C5, free hemoglobin, haptoglobin, CH50, AH50, sC5b-9, and PNH erythrocytes, granulocytes, monocytes, and exploratory research serum and plasma will be obtained on the specified days prior to Ravulizumab or the combination administration.
²⁸All biomarkers collected specifically on V3/day 1 must be collected pre-dose.
²⁹Whole blood samples for DNA extraction (optional) should be collected on day 1 (predose) but can be collected at a later study visit.
³⁰A Ravulizumab dose will be given at week 26 only if the patient intends to continue into OLE or R3918-PNH-2022. Patients who do not continue into a subsequent study with the Pozelimab/Cemdisiran combination will not receive a Ravulizumab dose at EOS.
³¹For patients planning to enroll in the OLE, day 1 of the R3918-PNH-2050 study must be scheduled 4 weeks after the week 24 dose of the combination treatment (i.e., 2 weeks after the week 26 EOS visit in the R3918-PNH-2021) to ensure no interruption in treatment administration.

TABLE 7-2
Schedule of Events for the Transition Period (In Patients who
Complete Ravulizumab in the OLTP and Plan to Enroll in the OLE)

| | | Study Procedure (Visit)¹ | |
|---|---|---|---|
| | | Transition Period (only patients who complete | |
| | | Ravulizumab OLTP and plan to enroll in OLE)² | |
| | | Visit # | |
| | | (4 weeks after last dose of | |
| | | Ravulizumab at OLTP week 26) | |
| | | TV1 | TV2 |
| Weeks (after last dose of Ravulizumab at OLTP week 26) | | 4t | 8t |
| Day | | 29t | 57t |
| Window (day) | | ±2 | ±2 |
| Baseline: | | | |
| Patient safety card for Neisseria meningitidis³ | | X | X |
| Treatment: | | | |
| IVRS/IWRS | | X | X |
| Ravulizumab arm | Pozelimab 60 mg/kg administration^{4, 6} | | X |
| | Pozelimab 400 mg SC^{5, 6} | | X |
| | Cemdisiran 200 mg SC^{5, 6} | X | X |

| | | |
|---|---|---|
| Concomitant meds and procedures | X | X |
| Transfusion record update | X | X |
| Antibiotics prophylaxis (recommended) ⁶ | X | X |
| Safety and Anthropometric: | | |
| Body weight | X | X |
| Vital signs | X | X |
| Adverse events | X | X |
| Breakthrough hemolysis assessment⁷ | X | X |
| Laboratory Testing⁸: | | |
| Hematology⁹ | X | X |
| Coagulation parameters | X | X |
| Blood chemistry (long panel) including LDH¹⁰ | X | X |
| Pregnancy test (applicable patients): urine (U) | U | U |
| Urinalysis | X | X |
| Pharmacokinetics and Total C5 Sampling: | | |
| Pozelimab PK sample¹¹ | | X |
| Ravulizumab PK sample | | X |
| Total C5 (plasma) | | X¹² |
| Biomarkers: | | |
| CH50 | X | X¹² |

Footnotes
¹When multiple procedures are performed on the same day, the sequence of procedures is as follows: vital signs, blood collection (first coagulation draw then chemistry draw followed by all other labs), and study drug administration. It is particularly important that the scheduled blood draws are obtained prior to the administration of Ravulizumab or Pozelimab and Cemdisiran, especially efficacy parameters such as LDH (i.e., measurements reflect a time point at the end of the dosing interval). During blood sample collection, handling, and processing, the same methodology will be applied across study visits, as best as possible, to preserve the quality of sample and avoid hemolysis during sample processing. If the investigator or sponsor suspects that the lab result is not an accurate reflection of the patient's condition, consideration should be given to repeating the lab sample if clinically warranted and, in all cases, where an LDH is ≥2 × ULN in association with potassium ≥6 mmol/L. Specific instructions for avoiding hemolysis are provided.
²Patients randomized to the Ravulizumab arm who complete the 26-week OLTP: If recruitment for the R3918-PNH-2022 is open and the patient has screened but was not eligible for the R3918-PNH-2022 study, the patient may instead proceed into the OLE study after completing the transition period. If recruitment for R3918-PNH-2022 is no longer open or the patient is not interested in participating in the R3918-PNH-2022, but is interested in participating in the OLE study, the patient may proceed directly into the transition period.
³Patient safety card for Neisseria meningitidis infection will be provided to the patient on any visit when needed. Site should review the instructions on the safety card with the patient at each visit.
⁴Patients randomized to the Ravulizumab arm, who complete the 26 weeks OLTP and plan to enroll into the OLE study, will transition from Ravulizumab to the combination treatment as follows: final Ravulizumab maintenance dose (per labeled weight-based dosing algorithm) will be administered IV at week 26 EOS visit in OLTP. Patients will receive Cemdisiran 200 mg SC on week 4t followed at week 8t by Pozelimab 60 mg/kg IV along with Pozelimab 400 mg SC and Cemdisiran 200 mg SC.
⁵All study treatments (i.e., Pozelimab, Cemdisiran, and Ravulizumab) should be administered as the last procedure after all blood sample collection and study assessments have been completed. After completion of administration of Pozelimab 60 mg/kg IV, patients should be monitored for at least 30 minutes. Patients should be monitored for at least 30 minutes after completion of the first Pozelimab SC dosing and the first Cemdisiran SC dosing.
⁶ Daily oral antibiotic prophylaxis against Neisseria meningitidis is recommended starting on the first day of dosing with study treatment and continuing until up to 52 weeks after discontinuation of Pozelimab and Cemdisiran. For post-treatment prophylaxis for Ravulizumab, consult the local prescribing information/national guidelines/local practice.
⁷If a patient is suspected of having a breakthrough hemolysis event, then in addition to the required laboratory collection, additional samples for CBC, coagulation parameters (including D-dimer), chemistry, reticulocyte count, total C5, CH50, drug concentrations of Pozelimab, Ravulizumab, and Cemdisiran, ADA (against Pozelimab), and exploratory research serum and plasma will be collected unless already noted in the schedule of events for that visit. If the suspected event does not occur at a scheduled visit, then an unscheduled visit should occur to evaluate the patient and to collect CBC, coagulation parameters (including D-dimer), chemistry, reticulocyte count, total C5, CH50 and drug concentrations of Ravulizumab/Pozelimab/Cemdisiran, and ADA (against Pozelimab). In addition, an exploratory research serum and plasma sample should be collected.
⁸Clinical lab samples will be collected first before study drug administration. The same methodology will be applied across study visits, as best as possible, to preserve the quality of sample and avoid hemolysis during sample processing.
⁹Hemoglobin will be assessed as part of the hematology analysis. Hematology sample should be collected before study treatment administration.
¹⁰Serum LDH, CRP, and bilirubin will be assessed as part of the blood chemistry analysis. Blood chemistry sample should be collected before study treatment administration.
¹¹Blood samples for Pozelimab PK will be obtained on the specified days prior to the Pozelimab IV dosing and also within 15 minutes after the end of the Pozelimab IV infusion.
¹²All biomarkers and total C5 collected specifically on TV2 must be collected pre-dose.

The FUP is for patients who discontinue treatment for any reason, including those who complete the OLTP but decline enrollment into the next study.

Patients in the Ravulizumab arm who complete OLTP (i.e., last dose of study drug at week 18 and EOS at week 26) will enter FUP at visit FU-3 (i.e., 12 weeks after last dose of study drug)

Patients in the Pozelimab/Cemdisiran arm who complete OLTP (i.e., last dose of study drug at week 24 and EOS at week 26) will enter FUP at visit FU-1 (i.e., 4 weeks after last dose of study drug)

For patients who discontinue and do not complete OLTP, they will have an ET visit. Thereafter, their entry point into the FUP will depend on the number of weeks that have elapsed after their final dose of study treatment

TABLE 7-3
Schedule of Events for the Safety Off-Treatment Follow-up Period

| | | | | | | Phone | Phone |
|---|---|---|---|---|---|---|---|
| | | | | | | visit | visit |
| Visit | FU-1 | FU-2 | FU-3 | FU-4 | FU-5 | FU-6 | FU-7 |
| Weeks (after the last dose of study drug) | 4 | 8 | 12 | 16 | 26 | 38 | 52 |
| Window (day) | ±10 | ±10 | ±10 | ±10 | ±10 | ±10 | ±10 |
| Body weight | X | X | X | X | X | | |
| Vital signs | X | X | X | X | X | | |
| Physical examination | | | X | | X | | |
| Concomitant meds/treatment | X | X | X | X | X | X | X |
| Adverse event reporting | X | X | X | X | X | X | X |
| Pregnancy reporting | X | X | X | X | X | X | X |
| Antibiotics prophylaxis (recommended)¹ | <-------------------------------------X---------------------------------------> | | | | | | |
| Patient safety card for Neisseria | X | X | X | X | X | X | X |
| meningitidis² | | | | | | | |

Laboratory Testing

| | | | | | |
|---|---|---|---|---|---|
| Hematology | X | X | X | X | X |
| Blood chemistry | X | X | X | X | X |

| | |
|---|---|
| Monthly urine pregnancy test (WOCBP)³ | <-------------------------------------X---------------------------------------> |

Pharmacokinetics, ADA, and Total C5 Sampling:

| | |
|---|---|
| Pozelimab PK sample | X |
| ADA sample for Pozelimab | X |
| ADA sample for Cemdisiran | X |
| Total C5 (plasma) | X |

Footnotes
¹Antibiotics prophylaxis (recommended): Daily oral antibiotic prophylaxis against Neisseria meningitidis is recommended, continuing until up to 52 weeks after discontinuation of Pozelimab and Cemdisiran. For post-treatment prophylaxis for Ravulizumab, follow the local prescribing information/national guidelines/local practice.
²Patient safety card for Neisseria meningitidis: Patient safety card for Neisseria meningitidis infection will be provided to the patient at FU-1 or any other visit when needed. Site should review the instructions on the safety card with the patient at each visit.
³Pregnancy testing: Monthly urine pregnancy testing will be conducted for WOCBP only. If performed via at-home testing kits, patients should be reminded to call the study staff each month with the results of their pregnancy test, and as soon as possible if their pregnancy test result is positive.

The study population will consist of adult male and female patients with confirmed diagnosis of PNH along with active signs and symptoms with evidence of hemolysis and are either complement inhibitor treatment-naive or have not recently received complement inhibitor treatment. Method of treatment assignment and stratification factors are described herein:

Patients will be randomized by an Interactive Web Response System (IWRS) in a 1:1 ratio to receive either the combination treatment with SC Pozelimab 400 mg and Cemdisiran 200 mg Q4W or IV Ravulizumab according to the labeled weight-based dosing algorithm. Stratified, blocked randomization will be performed using the following stratification factors:
RBC/whole blood transfusion within the past 1 year prior to randomization (yes/no) Note: Enrollment of patients who have had no RBC transfusion in the past 1 year before randomization will be capped at 20% of the overall enrolled population.
Screening visit LDH levels (2.0 to <4.0×ULN or ≥4.0×ULN)

Randomization blocking will be performed centrally.

Note: Enrollment of patients who have had no RBC transfusion in the past 1 year prior to day 1 will be capped at 20% of the overall enrolled population.

### Inclusion Criteria

A patient must meet the following criteria to be eligible for inclusion in the study.
1. Male or female 18 years of age or legal age of majority, whichever is greater, at the time of consent
2. Diagnosis of PNH confirmed by high-sensitivity flow cytometry testing with PNH granulocytes (i.e., polymorphonuclear neutrophils [PMNs]) or monocytes ≥5% at the screening visit
3. Active disease, as defined by the presence of 1 or more PNH-related signs or symptoms (e.g., fatigue, hemoglobinuria, abdominal pain, shortness of breath [dyspnea], anemia [hemoglobin ≤10 g/dL], history of a MAVE [including thrombosis], dysphagia, or erectile dysfunction) or history of RBC transfusion due to PNH within 3 months of the screening visit
4. LDH level ≥2×ULN at the screening visit
5. Willing and able to comply with clinic/remote visits and study-related procedures
6. Provide informed consent signed by study patient
7. Able to understand study-related questionnaires

### Exclusion Criteria

Note: If a patient screen fails, and if the study is still ongoing, they may be rescreened (up to 2 times) if the investigator determines the patient may be eligible upon rescreening.

A patient who meets any of the following criteria will be excluded from the study:

1. Prior treatment with Eculizumab within 3 months prior to screening, Ravulizumab within 6 months prior to screening, or other complement inhibitors within 5 half-lives of the respective agent prior to screening.
2. Receipt of an organ transplant, history of bone marrow transplantation or other hematologic transplant.
3. Body weight <40 kilograms at screening visit.
4. Current plans for modification (initiation, discontinuation, or dose/dosing interval change) of the following background concomitant medications, as applicable, during screening and treatment periods: erythropoietin, immunosuppressive drugs, corticosteroids, antithrombotic agents, anticoagulants, iron supplements, and folic acid.
5. Planned use of any complement inhibitor therapy other than study drugs during the treatment period.
6. Any of the following abnormalities at the screening visit (two repeat measurements are allowed per parameter during screening period):
a. Peripheral blood absolute neutrophil count (ANC)<500/μL (<0.5×10⁹/L) or
b. Peripheral blood platelet count <30,000/μL or
c. Peripheral blood reticulocyte count abnormality defined as <60,000/μL (<0.06×10⁶/μL, <60×10⁹/L)
   Note: A patient will not be excluded if upon repeat testing the parameter no longer meets the exclusion criterion.
   Note: Patients receiving acute treatment (e.g., platelet transfusions, granulocyte colony stimulating factors) for these conditions during screening and in the 1-month preceding screening will not be eligible
   7. Not meeting meningococcal vaccination requirements for Ravulizumab according to the current local prescribing information (where available) and at a minimum documentation of meningococcal vaccination within 5 years prior to screening visit.
   Note: Patients without prior vaccination will be eligible provided they are willing to undergo vaccination prior to initiation of study treatment and vaccination is documented prior to randomization.
   8. Any contraindication for receiving Neisseria meningitidis vaccination.
   9. Unable to take antibiotics for meningococcal prophylaxis (if required by local Ravulizumab prescribing information, where available, or national guidelines/local practice, or if necessary when vaccination is less than 2 weeks from study treatment initiation).
   10. Any active, ongoing infection or a recent infection requiring ongoing systemic treatment with antibiotics, antivirals, or antifungals within 2 weeks of screening or during the screening period.
   11. Documented history of systemic fungal disease or unresolved TB, or evidence of active or latent tuberculosis infection (LTBI) (i.e., if not having completed treatment for LTBI) during screening period. Assessment for active TB and LTBI should accord with local practice or guidelines, including those pertaining to risk assessment, and the use of tuberculin skin test or T-cell interferon-gamma release assay.
   12. Positive hepatitis B surface antigen or hepatitis C virus RNA during screening. Note: Cases with unclear interpretation should be discussed with the medical monitor.
   13. Patients with known HIV with history of opportunistic infections in the last 1 year, any history of HIV related malignancy, documented history of CD4 count <500 cells/μL or detectable viral load within the last 6 months (Note: CD4 count, and viral load must be available within the last 6 months, and may be conducted by a local laboratory during screening if needed)
   Note: Local testing for HIV may be conducted in patients if required locally or by local regulations.
   14. Documented* history of positive RT-PCR, antigen or serology test, or other health authority authorized test for SARS-CoV-2 and:
a. Have not recovered from COVID-19 (all COVID-19-related symptoms and major clinical findings which can potentially affect the safety of the patient should be resolved to baseline), and
b. Did not have 2 negative results from a health authority-authorized nucleic acid amplification (RT-PCR) test or other health authority authorized test for COVID-19 taken at least 48 hours apart prior to day 1.
   *Note. Screening for COVID-19 will not be performed as part of eligibility assessments for this study
   15. Known hereditary complement deficiency.
   16. Documented history of active, uncontrolled, ongoing systemic autoimmune diseases.
   17. Documented history of liver cirrhosis or patients with liver disease with evidence of current impaired liver function or patients with ALT or AST (unrelated to PNH or its complications) >3×ULN at the screening visit (if the AST or ALT returns >3×ULN, 1 repeat lab of the abnormal parameter(s) is allowed during screening).
   18. Patients with an eGFR of <30 mL/min/1.73 m² (according to Chronic Kidney Disease-Epidemiology Collaboration equation 2009) at screening visit (one repeat assessment allowed during screening).
   19. Recent unstable medical conditions, excluding PNH and PNH related complications, within the past 3 months prior to screening visit (e.g., myocardial infarction, congestive heart failure with New York Heart Association Class ≥III, serious uncontrolled cardiac arrhythmia, cerebrovascular accident, active gastrointestinal bleed).
   20. Anticipated need for major surgery during the study.
   21. History of cancer within the past 5 years, except for adequately treated basal cell skin cancer, squamous cell skin cancer, or in situ cervical cancer.
   22. Participation in another interventional clinical study or use of any experimental therapy within 30 days before screening visit or within 5 half-lives of that investigational product, whichever is greater, except for complement inhibitors.
   23. Known hypersensitivity to Eculizumab, Ravulizumab, Pozelimab, Cemdisiran or to any components of their respective formulations.
   24. Prior discontinuation of Eculizumab or Ravulizumab due to a safety reason or due to lack of efficacy.
   25. Patients with functional or anatomic asplenia.
   26. Any clinically significant abnormality identified at the time of screening that in the judgment of the investigator would preclude safe completion of the study or constrain endpoints assessment such as major systemic diseases, or patients with short life expectancy.
   27. Considered by the investigator or any sub-investigator as inappropriate for this study for any reason, e.g.,
a. Deemed unable to meet specific protocol requirements, such as scheduled visits.
b. Deemed unable to administer or tolerate need for chronic injections.
c. Presence of any other conditions (e.g., geographic, social) actual or anticipated, that the investigator feels would restrict or limit the patient's participation for the duration of the study.
d. Part of a vulnerable population such as the institutionalized (this may also include patients who are committed to an institution by order issued either by the judicial or the administrative authorities, as applicable).
e. Patient ineligible for clinical trial participation due to local regulations (e.g., under legal protection measures [such as L1121-8 or L1121-8-1 in France], etc).
   28. Members of the clinical site study team and/or his/her immediate family unless prior approval granted by the sponsor.
   29. Pregnant or breastfeeding women.
   30. Women of childbearing potential (WOCBP)* who are unwilling to practice highly effective contraception prior to the initial dose/start of the first treatment, during the study, and for at least 52 weeks after the last dose. Highly effective contraceptive measures include:
a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
b. intrauterine device (IUD); intrauterine hormone-releasing system (IUS)
c. bilateral tubal ligation or tubal occlusion;
d. vasectomized partner (provided that the male vasectomized partner is the sole sexual partner of the WOCBP study participant and that the vasectomized partner has obtained medical assessment of surgical success for the procedure) and/or
e. sexual abstinencet†, ‡
   *WOCBP are defined as women who are fertile following menarche until becoming postmenopausal, unless permanently sterile. Permanent sterilization methods include hysterectomy, bilateral salpingectomy, and bilateral oophorectomy.
   A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance.
   Pregnancy testing and contraception are required for WOCBP. Pregnancy testing and contraception are not required for women who are post-menopausal or permanently sterile.
   †Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the subject.
   ‡Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.
   31. Hemoglobin ≤7 g/dL (Note: A patient may receive a blood transfusion during the screening period and is eligible if repeat hemoglobin returns >7 g/dL prior to randomization. More than 2 repeat measurements are allowed.)

### Primary Objective

The primary objective of the study is to evaluate the effect on hemolysis and RBC transfusions over a 26-week treatment period of Pozelimab and Cemdisiran combination treatment versus Ravulizumab treatment in patients with active PNH who are complement inhibitor treatment-naive or have not recently received complement inhibitor therapy

### Secondary Objectives

The secondary objectives of the study are to:
Evaluate the effect of Pozelimab and Cemdisiran combination treatment versus Ravulizumab treatment on the following:
   Measures of hemolysis
   Transfusion parameters
   Hemoglobin levels
   Fatigue as assessed by Clinical Outcome Assessments (COAs)
   HRQoL as assessed by COAs
   Safety and tolerability
   Complement activation
To assess the concentrations of total Pozelimab and total Ravulizumab in serum and Cemdisiran and total C5 protein in plasma
To assess the immunogenicity of Pozelimab and Cemdisiran

### Exploratory Objectives

The exploratory objectives of the study are to explore the effect of Pozelimab and Cemdisiran combination treatment versus Ravulizumab treatment on the following:
Clinical thrombosis events
Renal function and renal injury biomarkers
Complement activation and hemolysis relevant to PNH and other related diseases
PNH clone size
COAs measuring treatment satisfaction (Treatment satisfaction questionnaire for medication [TSQM]), QoL (European Quality of Life-Five Dimension-Five Level Scale [EQ-5D-5L] and European organization for research and treatment of cancer quality-of-life questionnaire core 30 items [EORTC-QLQ-C30]), and PNH specific symptoms
Develop a molecular understanding of PNH, and related diseases and study mechanisms of action (including relationship to safety and efficacy) and complement pathway biology
To explore whether potential differences in patient efficacy and safety are associated with genotype and gene expression and to further study C5, PNH, or other conditions associated with complement-mediated injury, using optional whole blood DNA and RNA collection from consented patients

### Co-Primary Endpoints

The co-primary endpoints are:
Percent change in LDH from baseline to week 26
Transfusion avoidance (defined as not requiring an RBC transfusion as per protocol algorithm based on post-baseline hemoglobin values) from day 1 through week 26

### Key Secondary Endpoints

The key secondary endpoints are:
Maintenance of adequate control of hemolysis, defined as LDH≤1.5×ULN from week 8 through week 26, inclusive
Breakthrough hemolysis (defined as LDH≥2×ULN [subsequent to initial achievement of LDH≤1.5×ULN] concomitant with signs or symptoms associated with hemolysis) from post-baseline day 1 through week 26
Adequate control of hemolysis (defined as LDH≤1.5×ULN) from week 8 through week 26, inclusive
Hemoglobin stabilization (defined as patients who do not receive an RBC transfusion and have no decrease in hemoglobin level from baseline of ≥2 g/dL) from day 1 (post-baseline) through week 26
Normalization of LDH (defined as LDH≤1.0×ULN) between week 8 through week 26, inclusive
Change in fatigue as measured by the FACIT-Fatigue Scale from baseline to week 26
Change from baseline to week 26 in PF scores on the EORTC-QLQ-C30
Change from baseline to week 26 in global health status (GHS)/QoL scale score on the EORTC-QLC-C30

### Other Secondary Endpoints

The other secondary endpoints are:
Rate and number of units of RBC transfused per protocol algorithm from post-baseline day 1 through week 26
Time to first LDH≤1.5×ULN and ≤1.0×ULN
Percentage of days with LDH≤1.5×ULN between week 8 and week 26, inclusive
Change in hemoglobin levels from baseline to week 26
Incidence and severity of treatment emergent serious AEs, treatment-emergent adverse events (TEAEs) of special interest and TEAEs leading to treatment discontinuation over 26 weeks
Change and percent change in total CH50 from baseline to week 26
Concentration of total C5 in plasma assessed throughout the study
Concentrations of total Pozelimab in serum assessed throughout the study
Concentrations of Cemdisiran in plasma assessed throughout the study
Concentrations of total Ravulizumab in serum assessed throughout the study
Incidence of treatment emergent anti-drug antibodies (ADAs) to Pozelimab assessed throughout the study
Incidence of treatment emergent ADAs to Cemdisiran assessed throughout the study

### Exploratory Endpoints

The exploratory endpoints are:
Incidence of MAVE (defined as adverse event of special interest [AESI] that include thrombophlebitis/deep vein thrombosis, pulmonary embolus, myocardial infarction, unstable angina, renal vein or artery thrombosis, acute peripheral vascular occlusion, hepatic vein thrombosis, portal vein thrombosis, mesenteric/visceral vein thrombosis or infarction, mesenteric/visceral arterial thrombosis or infarction, transient ischemic attack, cerebral arterial occlusion/cerebrovascular accident, cerebral venous occlusion, gangrene [nontraumatic; non-diabetic], amputation [nontraumatic; non-diabetic]) from day 1 through week 26
Rate of breakthrough hemolysis from post-baseline day 1 to week 26
Change in renal function as measured by estimated glomerular filtration rate (eGFR) from baseline to week 26
Percent change in free hemoglobin from baseline to week 26
Change in haptoglobin from baseline to week 26
Change in total bilirubin from baseline to week 26
Change in reticulocyte count from baseline to week 26
Change and percent change in alternative pathway hemolytic activity assay (AH50) from baseline to week 26
Change in PNH erythrocytes, granulocytes, and monocytes from baseline to week 26
Comparison of treatment satisfaction as assessed by the TSQM at baseline versus treatment at week 26 (with Pozelimab/Cemdisiran or Ravulizumab)
Change in EQ-5D-5L scores from baseline to week 26
Change in PNH symptoms as measured by the PNH symptom-specific questionnaire scores from baseline to week 26
Change from baseline to week 26 in functional scale scores (Role Functioning, Emotional Functioning, Cognitive Functioning and Social Functioning) and symptom scale scores (Fatigue, Nausea and vomiting, Pain, Dyspnea, Insomnia, Appetite loss, Constipation, Diarrhea) of the EORTC-QLQ-C30
Improvement in GHS, functioning and symptoms as measured by the EORTC-QLQ-C30 from baseline to week 26
Time to first clinically meaningful improvement in HRQoL and symptoms as measured by EORTC-QLQ-C30 GHS functional and symptoms scales

### Laboratory Variables for the Assessment of Efficacy

Efficacy in this study is evaluated by the following laboratory assessments:
LDH (serum): LDH as a measure of intravascular hemolysis allows for an objective and precise means to gauge the control of intravascular hemolysis Hemoglobin: Hemolytic anemia is a hallmark of PNH

These laboratory variables are relevant to the characterization and disease mechanisms of PNH (Brodsky RA. Paroxysmal nocturnal hemoglobinuria. Blood 2014; 124(18):2804-2811).

### Transfusion Record

Hemolytic anemia is a clinical manifestation of PNH, and patients often require blood transfusion for symptomatic management. The frequency of blood transfusion has been used in other studies of PNH to assess efficacy (Hillmen P, Young NS, Schubert J, Brodsky RA, Socie G, Muus P, et al. The complement inhibitor Eculizumab in paroxysmal nocturnal hemoglobinuria. N Engl J Med 2006; 355(12):1233-1243) (Röth A, Egyed M, Ichikawa S, Kim J S, Nagy Z, Gasl Weisinger J, et al. The SMART Anti-hC5 Antibody (SKY59/RO7112689) Shows Good Safety and Efficacy in Patients with Paroxysmal Nocturnal Hemoglobinuria (PNH). Blood 2018; 132(Suppl 1):535.).

### Clinical Outcome Assessments

COAs include the following:
FACIT-Fatigue
EORTC-QLQ-C30
TSQM
EQ-5D-5L
PNH Symptom-Specific Questionnaire

### Safety and Anthropometric Variables

Safety and anthropometric variables in this study include:
Vaccination/revaccination for Neisseria meningitidis
Vaccination against Streptococcus pneumoniae and Haemophilus influenzae type B (if needed)
Concomitant medications and procedures
Height
Body weight
Vital Signs
Physical Examination
Electrocardiogram (ECG)
AEs
Breakthrough hemolysis assessment
Routine safety laboratory tests (hematology, chemistry, coagulation parameters, direct antiglobulin test, urinalysis, and pregnancy testing [for women of childbearing potential or WOCBP])

### Interim Results (Week 26)

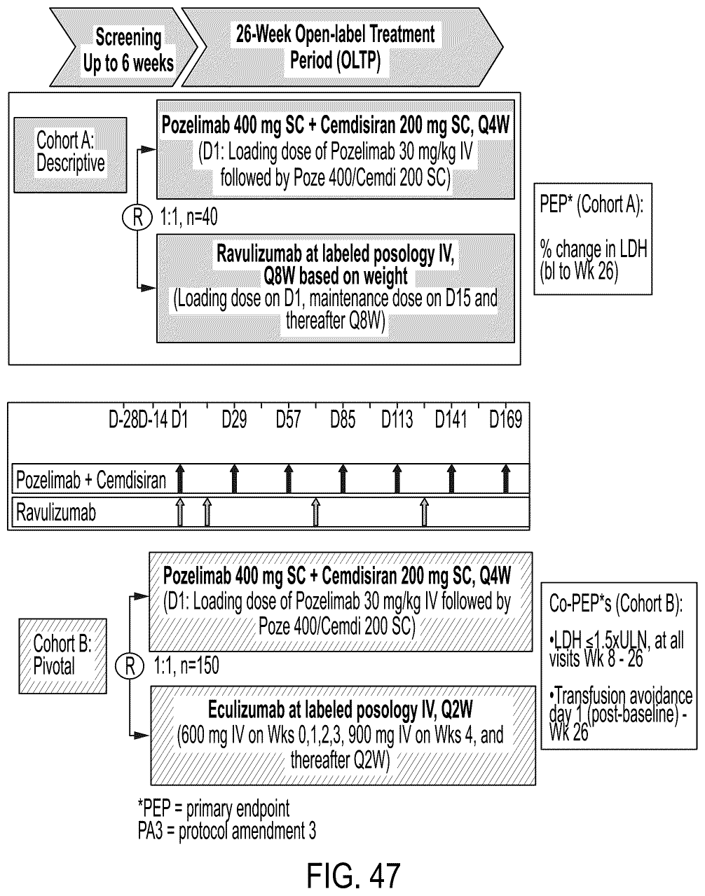

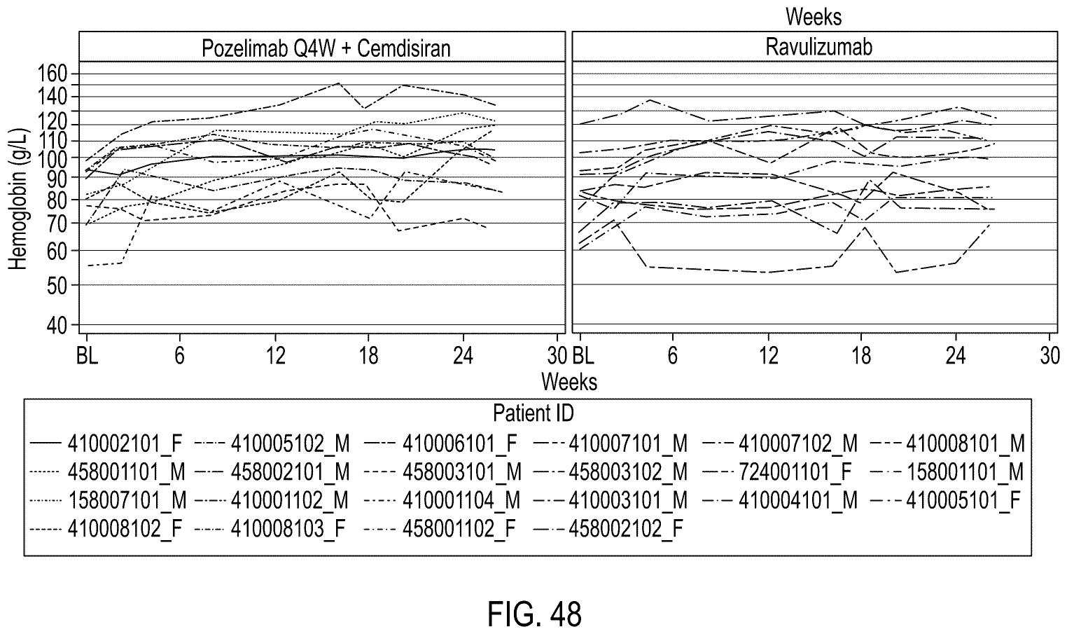

In this study, initially designed to study treatment of complement inhibitor naïve patients with PNH with the combination using Ravulizumab as a comparator was redesigned to use Eculizumab as a comparator. Cohort A (n=40) enrolled patients (1:1 randomization) to receive either Pozelimab/Cemdisiran combination treatment or Ravulizumab. Cohort B (n=150) enrolled patients (1:1 randomization) to receive Pozelimab/Cemdisiran combination treatment or Eculizumab. See FIG. 47.

Patients who received the combination treatment regimen (which may be referred to as “Pozelimab Q4W+Cemdisiran” or “combo” or “combination”) exhibited superior control over hemolysis, normalization of LDH and lack of breakthrough hemolysis, relative to patients who received only Ravulizumab monotherapy treatment regimen (“Ravulizumab”).

Adequate control of hemolysis refers to achievement of LDH≤1.5×ULN (considered clinically relevant threshold, ULN=upper limit of normal). Pharmacologic treatment is often initiated in symptomatic patients with LDH>1.5×ULN (LDH=lactate dehydrogenase). LDH s 1.5 xULN on complement inhibitor (CI) therapy correlates with improved symptoms, QoL and blood transfusions. Maintenance of adequate control of hemolysis refers to sustained reduction in LDH≤1.5×ULN at all visits from week 8 through week 26, inclusive, without an intervening event of breakthrough hemolysis. Normalization of LDH refers to achievement of LDH 51×ULN. Breakthrough hemolysis refers to an increase in LDH≥2×ULN after initial achievement of LDH≤1.5×ULN, plus signs or symptoms of hemolysis.

Twenty two patients completed the treatment period (11 in each arm) with 46 randomized at the time of the analysis (Table 7-4 and Table 7-5).

TABLE 7-4
Participant Disposition during the Open-Label
Treatment Period- Analysis Set for Cohort A

| | Pozelimab Q4W + | |
|---|---|---|
| | Cemdisiran | Ravulizumab |
| | (N = 24) | (N = 22) |
| Subjects Randomized | 24 (100%) | 22 (100%) |
| Subjects completed Week | 11 (45.8%) | 11 (50.0%) |
| 26 Visit | | |

TABLE 7-5
Study Participation-Analysis Set for Cohort A

| | Pozelimab Q4W + | |
|---|---|---|
| | Cemdisiran | Ravulizumab |
| | (N = 24) | (N = 22) |
| Time of Study | | |
| Participation (Days) [1] | | |
| n | 24 | 22 |
| Mean (SD) | 131.7 (67.42) | 156.2 (73.51) |
| Median | 148.5 | 179.5 |
| Q1:Q3 | 76:190 | 103:211 |
| Min:Max | 1:208 | 16:243 |
| Duration of Study | | |
| Participation, n (%) [1] | | |
| >=1 days | 24 (100%) | 22 (100%) |
| >=4 weeks | 22 (91.7%) | 21 (95.5%) |
| >=8 weeks | 20 (83.3%) | 19 (86.4%) |
| >=16 weeks | 15 (62.5%) | 16 (72.7%) |
| >=20 weeks | 14 (58.3%) | 14 (63.6%) |
| >=24 weeks | 11 (45.8%) | 12 (54.5%) |
| >=26 weeks | 11 (45.8%) | 11 (50.0%) |

[1] Time on Study is defined as: [date of last study contact] − [date of first dose] + 1

Baseline characteristics were generally similar in both arms (Table 7-6).

TABLE 7-6
Baseline characteristics - Combo (Pozelimab
Q4W + Cemdisiran) vs Ravulizumab, interim

| | Pozelimab Q4W + | |
|---|---|---|
| | Cemdisiran | Ravulizumab |
| | (n = 24) | (n = 22) |
| Age (mean, years)(SD) | 46.7 | 41.1 |
| | (16.2) | (17.5) |
| % male | 50% | 54.5% |
| LDH, mean (×ULN) (SD) | 6.6 | 5.9 |
| | (3.1) | (3.6) |
| Hemoglobin (mean, g/L) | 84.6 | 87.9 |
| PNH RBC clones, mean | 34% | 34% |
| (SD) | (22.7) | (29.7) |
| PNH monocyte clones, | 85% | 84% |
| mean | | |
| PNH granulocyte clones, | 72% | 73% |
| mean | | |
| Total bilirubin (mean, | 31 | 30 |
| umol/L) (SD) | (26.6) | (22.4) |
| Reticulocytes (mean, | 210 | 172 |
| 10⁹/L)* | (125.2) | (67.3) |
| Transfusions in prior | 75%, 6 | 73%, 6.5 |
| year (% of pts, # of | | |
| transfusions) | | |
| AA/MDS\hat{ }(% of pts, # pts) | 17%, 4 | 23%, 5 |

*median reticulocytes: 175 and 174 × 10⁹/L respectively
\hat{ }1 MDS patient in each arm

Hemoglobin (Hb) stabilization & transfusion avoidance was similar in week 26 completers among patients on combo vs Ravulizumab. See Tables 7-7 A-D.

Ninety-one % of the Combo patients vs 73% of the Ravulizumab patients maintained adequate control of hemolysis (LDH≤1.5×ULN) (Table 7-7). No patient (n=46) met the protocol criteria for breakthrough hemolysis. One patient had a bump in LDH levels on the combo due to viral fever D179 to 181 (LDH increase at D182, V13).

TABLE 7-7A
Patients who Maintained Adequate Control
of Hemolysis-Analysis Set for Cohort A

| | Pozelimab Q4W + | |
|---|---|---|
| | Cemdisiran | Ravulizumab |
| | (n = 24) | (n = 22) |
| Patients who completed the OLTP- | 10/11 (90.9%) | 8/11 (72.7%) |
| responders | | |
| Patients still on-going in the OLTP | | |
| Non-responders | 1/13 (7.69%) | 1/11 (9.09%) |
| Status to be | 12/13 (92.3%) | 10/11 (90.9%) |
| determined | | |

TABLE 7-7B
Patients with Hemoglobin Stabilization-Analysis Set for Cohort A

| | Pozelimab Q4W + | |
|---|---|---|
| | Cemdisiran | Ravulizumab |
| | (N = 24) | (N = 22) |
| Patients who completed the OLTP | | |
| Responders | 6/11 (54.5%) | 6/11 (54.5%) |
| Non-Responders | 5/11 (45.5%) | 5/11 (45.5%) |
| Patients still on-going in the OLTP | | |
| Non-Responders | 7/13 (53.8%) | 3/11 (27.3%) |
| Status to be determined | 6/13 (46.2%) | 8/11 (72.7%) |

A responder is a patient who does not receive an Per-protocol RBC transfusion and has no decrease in hemoglobin level from baseline of >=2 g/dL from day 1 (post-baseline) through week 26. Hemoglobin stabilization: patients who do not receive an RBC transfusion and have no decrease in Hb from baseline of ≥2 g/dL from day 1 (post-baseline) through week 26

TABLE 7-7C
Patients who Achieved Transfusion Avoidance Per
Protocol Algorithm-Analysis Set for Cohort A

| | Pozelimab Q4W + | |
|---|---|---|
| | Cemdisiran | Ravulizumab |
| | (N = 24) | (N = 22) |
| Patients who completed the OLTP | | |
| Responders | 6/11 (54.5%) | 6/11 (54.5%) |
| Non-Responders | 5/11 (45.5%) | 5/11 (45.5%) |
| Patients still on-going in the OLTP | | |
| Non-Responders | 7/13 (53.8%) | 3/11 (27.3%) |
| Status to be determined | 6/13 (46.2%) | 8/11 (72.7%) |

Transfusion avoidance: not requiring an RBC transfusion per protocol algorithm based on post-baseline Hb values from day 1 through week 26.

Transfusions can be driven by:
Hemolysis (IVH (intravascular hemolysis), EVH (extravascular hemolysis))*
Bone marrow insufficiency
Other (e.g., blood loss)
   May be imbalance in other factors given small sample size.

TABLE 7-7D
Transfusion Summary

| | | On study pts |
|---|---|---|
| | | with |
| | On study pts | transfusion, |
| | with | all (whether |
| | transfusion | or not |
| | (completers) | criteria met) |
| Combination | 36% | 46% |
| Ravulizumab | 36% | 32% |

There was a higher proportion of Combo patients that achieved adequate control of hemolysis (LDH e 1.5×ULN By Visit) relative to Ravulizumab patients. The LDH measurements of each patient per visit are set forth in Table 7-8. LDH measurements that were above the reference range are indicated with an “(H)” and LDH measurements that were below the reference range are indicated with an “(L)”.

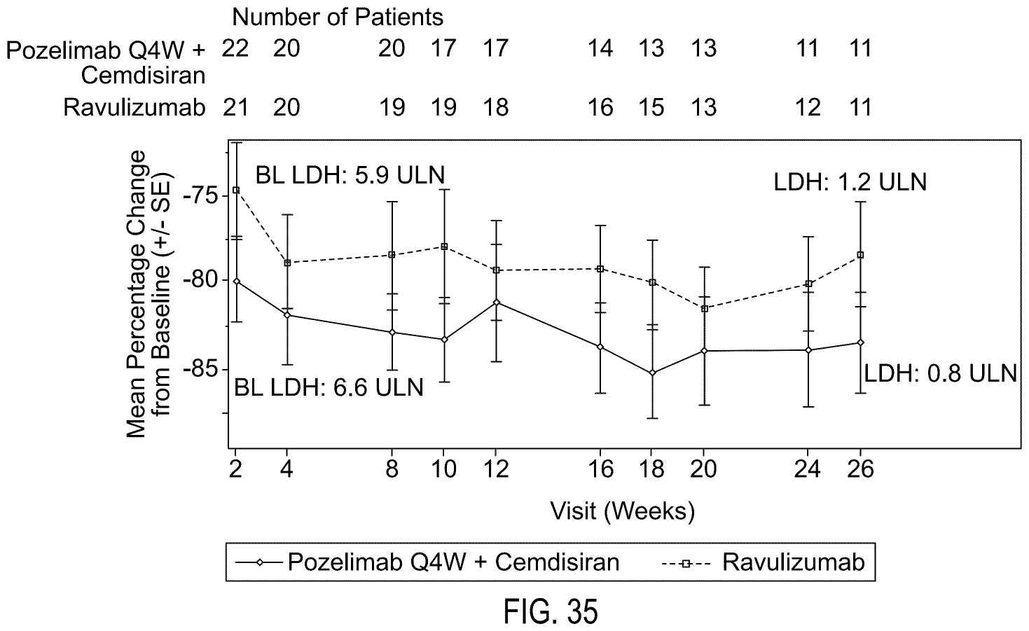

The mean percentage change from baseline in LDH among Combo patients and Ravulizumab patients is graphically summarized in FIG. 35. The Combo patients achieved a greater percent change and lower mean LDH from baseline to week 26 relative to that of Ravulizumab patients. The baseline LDH among those completing week 26 was: Combo: 6.5×ULN and Ravulizumab: 5.7×ULN. The mean % change from baseline at week 26 was: Combo: −83.8 and Ravulizumab: −78.5.

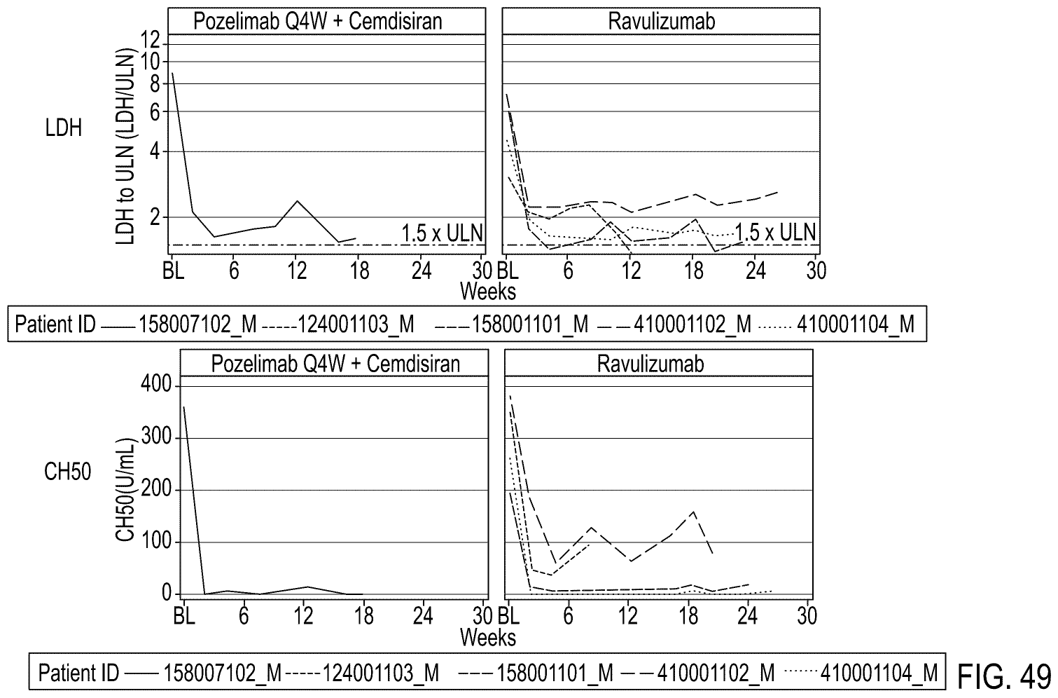

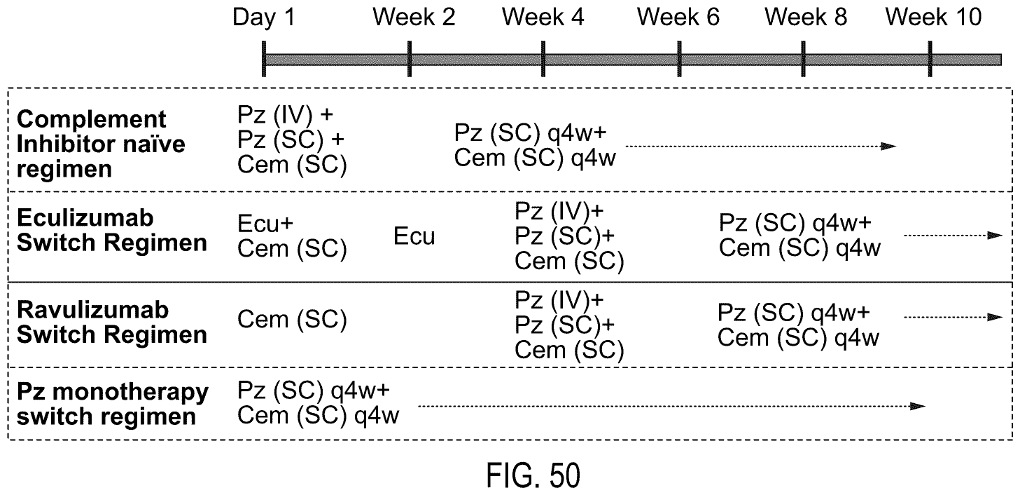

A comparison between the LDH and CH56 values of inadequate responders from each arm is set forth in FIG. 49.

TABLE 7-8
Clinical Laboratory Tests-LDH (Full Analysis Set)

| | | | | |
|---|---|---|---|---|
| | | | Reference | |
| Subject ID | Age/Sex | Visit | Range | Result |

Pozelimab q4w + Cemdisiran

| | | | | |
|---|---|---|---|---|
| 158003102 | 39/F | Screening Visit 1 | 135-330 U/L | 717(H) |
| | | Visit 3 | 135-330 U/L | 695(H) |
| | | Visit 4 | 135-330 U/L | 251 |
| | | Visit 5 | 135-330 U/L | 395(H) |
| | | Visit 6 | 135-330 U/L | 244 |
| | | Visit 7 | 135-330 U/L | 224 |
| | | Visit 8 | 135-330 U/L | 297 |
| 158007102 | 66/M | Screening Visit 1 | 135-281 U/L | 2580(H) |
| | | Visit 3 | 135-281 U/L | 2520(H) |
| | | Visit 4 | 135-281 U/L | 597(H) |
| | | Visit 5 | 135-281 U/L | 456(H) |
| | | Visit 6 | 135-281 U/L | 500(H) |
| | | Visit 7 | 135-281 U/L | 515(H) |
| | | Visit 8 | 135-281 U/L | 664(H) |
| | | Visit 9 | 135-281 U/L | 434(H) |
| | | Visit 10 | 135-281 U/L | 457(H) |
| 380003101 | 40/F | Screening Visit 1 | 135-330 U/L | 1650(H) |
| | | Visit 3 | 135-330 U/L | 1695(H) |
| | | Visit 4 | 135-330 U/L | 327 |
| | | Visit 5 | 135-330 U/L | 291 |
| | | Visit 6 | 135-330 U/L | 307 |
| 410002101 | 65/F | Screening Visit 1 | 135-330 U/L | 957(H) |
| | | Visit 3 | 135-330 U/L | 1010(H) |
| | | Visit 4 | 135-330 U/L | 298 |
| | | Visit 5 | 135-330 U/L | 266 |
| | | Visit 6 | 135-330 U/L | 260 |
| | | Visit 7 | 135-330 U/L | 248 |
| | | Visit 8 | 135-330 U/L | 255 |
| | | Visit 9 | 135-330 U/L | 259 |
| | | Visit 10 | 135-330 U/L | 250 |
| | | Visit 11 | 135-330 U/L | 282 |
| | | Visit 12 | 135-330 U/L | 283 |
| | | Visit 13 | 135-330 U/L | 280 |
| 410005102 | 61/M | Screening Visit 1 | 135-281 U/L | 2565(H) |
| | | Visit 3 | 135-281 U/L | 3000(H) |
| | | Visit 4 | 135-281 U/L | 276 |
| | | Visit 5 | 135-281 U/L | 190 |
| | | Visit 6 | 135-281 U/L | 159 |
| | | Visit 7 | 135-281 U/L | 183 |
| | | Visit 8 | 135-281 U/L | 219 |
| | | Visit 9 | 135-281 U/L | 184 |
| | | Visit 10 | 135-281 U/L | 186 |
| | | Visit 11 | 135-281 U/L | 191 |
| | | Visit 12 | 135-281 U/L | 176 |
| | | Visit 13 | 135-281 U/L | 169 |
| 410005105 | 39/M | Screening Visit 1 | 135-281 U/L | 1065(H) |
| | | Visit 3 | 135-281 U/L | 1165(H) |
| | | Visit 4 | 135-281 U/L | 250 |
| | | Visit 5 | 135-281 U/L | 158 |
| | | Visit 6 | 135-281 U/L | 167 |
| 410006101 | 36/F | Screening Visit 1 | 135-330 U/L | 1544(H) |
| | | Visit 3 | 135-330 U/L | 1581(H) |
| | | Visit 4 | 135-330 U/L | 232 |
| | | Visit 5 | 135-330 U/L | 194 |
| | | Visit 6 | 135-330 U/L | 223 |
| | | Visit 7 | 135-330 U/L | 186 |
| | | Visit 8 | 135-330 U/L | 177 |
| | | Visit 9 | 135-330 U/L | 212 |
| | | Visit 10 | 135-330 U/L | 172 |
| | | Visit 11 | 135-330 U/L | 185 |
| | | Visit 12 | 135-330 U/L | 177 |
| | | Visit 13 | 135-330 U/L | 165 |
| 410007101 | 40/M | Screening Visit 1 | 135-281 U/L | 1586(H) |
| | | Visit 3 | 135-281 U/L | 1485(H) |
| | | Visit 4 | 135-281 U/L | 280 |
| | | Visit 5 | 135-281 U/L | 202 |
| | | Visit 6 | 135-281 U/L | 217 |
| | | Visit 6 | 135-281 U/L | 216 |
| | | Visit 7 | 135-281 U/L | 229 |
| | | Visit 8 | 135-281 U/L | 200 |
| | | Visit 9 | 135-281 U/L | 198 |
| | | Visit 10 | 135-281 U/L | 200 |
| | | Visit 11 | 135-281 U/L | 202 |
| | | Visit 12 | 135-281 U/L | 233 |
| | | Visit 13 | 135-281 U/L | 230 |
| 410007102 | 58/M | Screening Visit 1 | 135-281 U/L | 1845(H) |
| | | Visit 3 | 135-281 U/L | 3015(H) |
| | | Visit 4 | 135-281 U/L | 300(H) |
| | | Visit 5 | 135-281 U/L | 175 |
| | | Visit 6 | 135-281 U/L | 204 |
| | | Visit 7 | 135-281 U/L | 211 |
| | | Visit 8 | 135-281 U/L | 203 |
| | | Visit 9 | 135-281 U/L | 198 |
| | | Visit 10 | 135-281 U/L | 204 |
| | | Visit 11 | 135-281 U/L | 182 |
| | | Visit 12 | 135-281 U/L | 200 |
| | | Visit 13 | 135-281 U/L | 201 |
| 410008101 | 73/M | Screening Visit 1 | 135-281 U/L | 949(H) |
| | | Visit 3 | 135-281 U/L | 699(H) |
| | | Visit 4 | 135-281 U/L | 279 |
| | | Visit 5 | 135-281 U/L | 274 |
| | | Visit 6 | 135-281 U/L | 232 |
| | | Visit 7 | 135-281 U/L | 273 |
| | | Visit 8 | 135-281 U/L | 331(H) |
| | | Visit 8 | 135-281 U/L | 392(H) |
| | | Visit 9 | 135-281 U/L | 278 |
| | | Visit 10 | 135-281 U/L | 273 |
| | | Visit 11 | 135-281 U/L | 305(H) |
| | | Unscheduled Visit 111.01 | 135-281 U/L | 250 |
| | | Visit 12 | 135-281 U/L | 280 |
| | | Visit 13 | 135-281 U/L | 239 |
| 458001101 | 44/M | Screening Visit 1 | 135-281 U/L | 2145(H) |
| | | Visit 3 | 135-281 U/L | 1950(H) |
| | | Visit 4 | 135-281 U/L | 295(H) |
| | | Visit 5 | 135-281 U/L | 197 |
| | | Visit 6 | 135-281 U/L | 191 |
| | | Visit 7 | 135-281 U/L | 277 |
| | | Visit 8 | 135-281 U/L | 201 |
| | | Visit 9 | 135-281 U/L | 236 |
| | | Visit 10 | 135-281 U/L | 236 |
| | | Visit 11 | 135-281 U/L | 216 |
| | | Visit 12 | 135-281 U/L | 208 |
| | | Visit 13 | 135-281 U/L | 238 |
| 458002101 | 46/M | Screening Visit 1 | 135-281 U/L | 2760(H) |
| | | Visit 3 | 135-281 U/L | 2370(H) |
| | | Visit 4 | 135-281 U/L | 251 |
| | | Visit 5 | 135-281 U/L | 208 |
| | | Visit 6 | 135-281 U/L | 313(H) |
| | | Visit 6 | 135-281 U/L | 257 |
| | | Visit 7 | 135-281 U/L | 323(H) |
| | | Visit 8 | 135-281 U/L | 274 |
| | | Visit 9 | 135-281 U/L | 263 |
| | | Visit 10 | 135-281 U/L | 201 |
| | | Visit 11 | 135-281 U/L | 209 |
| | | Visit 12 | 135-281 U/L | 210 |
| | | Visit 13 | 135-281 U/L | 212 |
| | | Follow-up 1 | 135-281 U/L | 200 |
| 458002103 | 43/F | Screening Visit 1 | 135-330 U/L | 2340(H) |
| | | Unscheduled Visit 101.01 | 135-330 U/L | 3105(H) |
| | | Visit 3 | | |
| | | Unscheduled Visit 104.02 | 135-330 U/L | 520(H) |
| | | Visit 4 | 135-330 U/L | 390(H) |
| | | Visit 5 | 135-330 U/L | 221 |
| | | Visit 6 | 135-330 U/L | 145 |
| | | Visit 7 | 135-330 U/L | 157 |
| | | Visit 8 | 135-330 U/L | 142 |
| 458003101 | 39/M | Screening Visit 1 | 135-281 U/L | 2565(H) |
| | | Unscheduled Visit 102.01 | 135-281 U/L | 3330(H) |
| | | Visit 3 | 135-281 U/L | 2550(H) |
| | | Visit 4 | 135-281 U/L | 328(H) |
| | | Visit 5 | 135-281 U/L | 209 |
| | | Visit 6 | 135-281 U/L | 175 |
| | | Visit 7 | 135-281 U/L | 199 |
| | | Visit 8 | 135-281 U/L | 193 |
| | | Visit 9 | 135-281 U/L | 256 |
| | | Visit 10 | 135-281 U/L | 187 |
| | | Visit 11 | 135-281 U/L | 220 |
| | | Visit 12 | 135-281 U/L | 177 |
| | | Visit 13 | 135-281 U/L | 177 |
| 458003102 | 29/M | Screening Visit 1 | 135-281 U/L | 1755(H) |
| | | Visit 3 | 135-281 U/L | 2145(H) |
| | | Visit 4 | 135-281 U/L | 231 |
| | | Visit 5 | 135-281 U/L | 179 |
| | | Visit 6 | 135-281 U/L | 174 |
| | | Visit 7 | 135-281 U/L | 169 |
| | | Visit 8 | 135-281 U/L | 17+ |
| | | Visit 9 | 135-281 U/L | 196 |
| | | Visit 10 | 135-281 U/L | 176 |
| | | Visit 11 | 135-281 U/L | 175 |
| | | Visit 12 | 135-281 U/L | 180 |
| | | Visit 13 | 135-281 U/L | 449(H) |
| 616002101 | 21/F | Screening Visit 1 | 135-330 U/L | 677(H) |
| | | Unscheduled Visit 102.01 | 135-330 U/L | 870(H) |
| | | Visit 3 | 135-330 U/L | 864(H) |
| | | Visit 4 | 135-330 U/L | 301 |
| | | Visit 5 | 135-330 U/L | 242 |
| | | Visit 6 | 135-330 U/L | 237 |
| | | Visit 7 | 135-330 U/L | 231 |
| | | Visit 8 | 135-330 U/L | 248 |
| 702001102 | 47/M | Screening Visit 1 | 135-281 U/L | 2535(H) |
| | | Visit 3 | 135-281 U/L | 3270(H) |
| | | Visit 4 | 135-281 U/L | 258 |
| | | Visit 5 | 135-281 U/L | 141 |
| | | Visit 6 | 135-281 U/L | 176 |
| | | Visit 7 | 135-281 U/L | 152 |
| | | Visit 8 | 135-281 U/L | 165 |
| | | Visit 9 | 135-281 U/L | 164 |
| | | Visit 10 | 135-281 U/L | 164 |
| | | Visit 11 | 135-281 U/L | 177 |
| 724001101 | 48/F | Screening Visit 1 | 135-330 U/L | 1206(H) |
| | | Visit 3 | 135-330 U/L | 805(H) |
| | | Visit 4 | 135-330 U/L | 395(H) |
| | | Visit 5 | 135-330 U/L | 312 |
| | | Visit 6 | 135-330 U/L | 297 |
| | | Visit 7 | 135-330 U/L | 246 |
| | | Visit 8 | 135-330 U/L | 255 |
| | | Visit 9 | 135-330 U/L | 246 |
| | | Visit 10 | 135-330 U/L | 203 |
| | | Visit 11 | 135-330 U/L | 262 |
| | | Visit 12 | 135-330 U/L | 239 |
| | | Visit 13 | 135-330 U/L | 230 |
| 724002101 | 87/F | Screening Visit 1 | 135-330 U/L | >1260(H) |
| | | Visit 3 | 135-330 U/L | 1218(H) |
| | | Visit 4 | 135-330 U/L | 319 |
| | | Visit 5 | 135-330 U/L | 248 |
| | | Visit 6 | 135-330 U/L | 246 |
| | | Visit 8 | 135-330 U/L | 229 |
| | | Visit 9 | 135-330 U/L | 263 |
| | | Visit 11 | 135-330 U/L | 243 |
| 764003102 | 49/F | Screening Visit 1 | 135-330 U/L | 2385(H) |
| 764004101 | 56/M | Screening Visit 1 | 135-281 U/L | 2490(H) |
| | | Visit 3 | 135-281 U/L | 2565(H) |
| | | Visit 4 | 135-281 U/L | 268 |
| 764005101 | 50/F | Screening Visit 1 | 135-330 U/L | 2550(H) |
| | | Visit 3 | 135-330 U/L | 3720(H) |
| | | Visit 4 | 135-330 U/L | 228 |
| 792001103 | 21/F | Screening Visit 1 | 135-330 U/L | 1183(H) |
| | | Visit 3 | 135-330 U/L | 1875(H) |
| | | Visit 4 | 135-330 U/L | 340(H) |
| | | Visit 5 | 135-330 U/L | 262 |
| 840001102 | 23/F | Screening Visit 1 | 135-330 U/L | 2640(H) |
| | | Visit 3 | 135-330 U/L | 2145(H) |
| | | Visit 6 | 135-330 U/L | 387(H) |
| | | Visit 7 | 135-330 U/L | 217 |

Ravulizumab

| | | | | |
|---|---|---|---|---|
| 124001102 | 58/M | Screening Visit 1 | 135-281 U/L | 2340(H) |
| | | Screening Visit 1 | 135-281 U/L | 1015(H) |
| | | Visit 3 | 135-281 U/L | 1380(H) |
| | | Visit 4 | 135-281 U/L | 273 |
| | | Visit 5 | 135-281 U/L | 245 |
| | | Visit 6 | 135-281 U/L | 213 |
| | | Visit 7 | 135-281 U/L | 232 |
| | | Visit 8 | 135-281 U/L | 225 |
| | | Visit 9 | 135-281 U/L | 220 |
| | | Visit 10 | 135-281 U/L | 232 |
| 124001103 | 21/M | Screening Visit 1 | 135-281 U/L | 1380(H) |
| | | Visit 3 | 135-281 U/L | 860(H) |
| | | Visit 4 | 135-281 U/L | 588(H) |
| | | Visit 5 | 135-281 U/L | 553(H) |
| | | Visit 6 | 135-281 U/L | 618(H) |
| | | Visit 7 | 135-281 U/L | 638(H) |
| | | Visit 8 | 135-281 U/L | 504(H) |
| | | Visit 9 | 135-281 U/L | 393(H) |
| 124001105 | 36/M | Screening Visit 1 | 135-281 U/L | 520(H) |
| | | Screening Visit 1 | 135-281 U/L | 571(H) |
| | | Visit 3 | 135-281 U/L | 515(H) |
| 158001101 | 34/M | Screening Visit 1 | 135-281 U/L | 1890(H) |
| | | Visit 3 | 135-281 U/L | 1710(H) |
| | | Visit 4 | 135-281 U/L | 503(H) |
| | | Visit 5 | 135-281 U/L | 407(H) |
| 158001101 | 34/M | Visit 6 | 135-281 U/L | 442(H) |
| | | Visit 7 | 135-281 U/L | 538(H) |
| | | Visit 8 | 135-281 U/L | 440(H) |
| | | Visit 9 | 135-281 U/L | 463(H) |
| | | Visit 10 | 135-281 U/L | 557(H) |
| | | Visit 11 | 135-281 U/L | 394(H) |
| | | Visit 12 | 135-281 U/L | 459(H) |
| | | Visit 13 | 135-281 U/L | 488(H) |
| | | Tv1r | 135-281 U/L | 482(H) |
| | | Unscheduled Visit 215.01 | 135-281 U/L | 329(H) |
| 158003101 | 66/M | Screening Visit 1 | 135-281 U/L | 1028(H) |
| | | Visit 3 | 135-281 U/L | 1044(H) |
| | | Visit 4 | 135-281 U/L | 246 |
| | | Visit 5 | 135-281 U/L | 179 |
| | | Visit 6 | 135-281 U/L | 162 |
| | | Unscheduled Visit 107.01 | 135-281 U/L | 193 |
| | | Visit 8 | 135-281 U/L | 191 |
| | | Visit 9 | 135-281 U/L | 171 |
| | | Visit 10 | 135-281 U/L | 188 |
| 158007101 | 20/M | Screening Visit 1 | 135-281 U/L | 2115(H) |
| | | Visit 3 | 135-281 U/L | 2085(H) |
| | | Visit 4 | 135-281 U/L | 472(H) |
| | | Visit 5 | 135-281 U/L | 292(H) |
| | | Visit 6 | 135-281 U/L | 307(H) |
| | | Visit 7 | 135-281 U/L | 248 |
| | | Visit 8 | 135-281 U/L | 242 |
| | | Visit 9 | 135-281 U/L | 376(H) |
| | | Visit 10 | 135-281 U/L | 226 |
| | | Visit 11 | 135-281 U/L | 239 |
| | | Visit 12 | 135-281 U/L | 297(H) |
| | | Visit 13 | 135-281 U/L | 277 |
| | | Unscheduled Visit 113.01 | 135-281 U/L | 225 |
| 392004101 | 75/M | Screening Visit 1 | 135-281 U/L | 1538(H) |
| | | Visit 3 | 135-281 U/L | 1236(H) |
| | | Visit 4 | 135-281 U/L | 337(H) |
| | | Visit 5 | 135-281 U/L | 276 |
| 410001102 | 62/M | Screening Visit 1 | 135-281 U/L | 1380(H) |
| | | Visit 3 | 135-281 U/L | 2055(H) |
| | | Visit 4 | 135-281 U/L | 623(H) |
| | | Visit 5 | 135-281 U/L | 619(H) |
| | | Visit 6 | 135-281 U/L | 664(H) |
| | | Visit 7 | 135-281 U/L | 661(H) |
| | | Visit 8 | 135-281 U/L | 594(H) |
| | | Visit 9 | 135-281 U/L | 684(H) |
| | | Visit 10 | 135-281 U/L | 716(H) |
| | | Visit 11 | 135-281 U/L | 642(H) |
| | | Visit 12 | 135-281 U/L | 681(H) |
| | | Visit 13 | 135-281 U/L | 735(H) |
| 410001104 | 33/M | Screening Visit 1 | 135-281 U/L | 1254(H) |
| | | Screening Visit 1 | 135-281 U/L | 1345(H) |
| | | Visit 3 | 135-281 U/L | 1252(H) |
| | | Visit 4 | 135-281 U/L | 551(H) |
| | | Visit 5 | 135-281 U/L | 465(H) |
| | | Visit 6 | 135-281 U/L | 450(H) |
| | | Visit 7 | 135-281 U/L | 448(H) |
| | | Visit 8 | 135-281 U/L | 509(H) |
| | | Visit 9 | 135-281 U/L | 480(H) |
| | | Visit 10 | 135-281 U/L | 493(H) |
| | | Visit 11 | 135-281 U/L | 461(H) |
| | | Visit 12 | 135-281 U/L | 485(H) |
| | | Visit 13 | 135-281 U/L | 494(H) |
| 410003101 | 47/M | Screening Visit 1 | 135-281 U/L | 2010(H) |
| | | Visit 3 | 135-281 U/L | 2025(H) |
| | | Visit 4 | 135-281 U/L | 188 |
| | | Visit 5 | 135-281 U/L | 158 |
| | | Visit 6 | 135-281 U/L | 169 |
| | | Visit 7 | 135-281 U/L | 168 |
| | | Visit 8 | 135-281 U/L | 166 |
| | | Visit 8 | 135-281 U/L | 130(L) |
| | | Visit 9 | 135-281 U/L | 149 |
| | | Visit 10 | 135-281 U/L | 165 |
| | | Visit 11 | 135-281 U/L | 154 |
| | | Visit 12 | 135-281 U/L | 151 |
| | | Visit 13 | 135-281 U/L | 183 |
| | | Tv1r | 135-281 U/L | 178 |
| | | Tv2r | 135-281 U/L | 149 |
| 410004101 | 67/M | Screening Visit 1 | 135-281 U/L | 684(H) |
| | | Visit 3 | 135-281 U/L | 1125(H) |
| | | Visit 4 | 135-281 U/L | 313(H) |
| | | Visit 5 | 135-281 U/L | 272 |
| | | Visit 6 | 135-281 U/L | 253 |
| | | Visit 7 | 135-281 U/L | 236 |
| | | Visit 8 | 135-281 U/L | 224 |
| | | Visit 9 | 135-281 U/L | 251 |
| | | Visit 10 | 135-281 U/L | 235 |
| | | Visit 11 | 135-281 U/L | 225 |
| | | Visit 12 | 135-281 U/L | 229 |
| | | Visit 13 | 135-281 U/L | 251 |
| 410005101 | 20/F | Screening Visit 1 | 135-330 U/L | 2355(H) |
| | | Visit 3 | 135-330 U/L | 2265(H) |
| | | Visit 4 | 135-330 U/L | 400(H) |
| | | Visit 5 | 135-330 U/L | 333(H) |
| | | Visit 6 | 135-330 U/L | 327 |
| | | Visit 7 | 135-330 U/L | 328 |
| | | Visit 8 | 135-330 U/L | 279 |
| | | Visit 9 | 135-330 U/L | 311 |
| | | Visit 10 | 135-330 U/L | 303 |
| | | Visit 11 | 135-330 U/L | 259 |
| | | Visit 12 | 135-330 U/L | 256 |
| | | Visit 13 | 135-330 U/L | 272 |
| | | Unscheduled Visit 214.01 | 135-330 U/L | 277 |
| | | Unscheduled Visit 215.01 | 135-330 U/L | 307 |
| 410005103 | 23/F | Screening Visit 1 | 135-330 U/L | 2160(H) |
| | | Visit 3 | 135-330 U/L | 1684(H) |
| | | Visit 4 | 135-330 U/L | 331(H) |
| | | Visit 5 | 135-330 U/L | 280 |
| | | Visit 6 | 135-330 U/L | 267 |
| | | Visit 7 | 135-330 U/L | 257 |
| | | Visit 8 | 135-330 U/L | 217 |
| | | Visit 9 | 135-330 U/L | 214 |
| | | Visit 10 | 135-330 U/L | 216 |
| | | Visit 11 | 135-330 U/L | 217 |
| 410008102 | 25/F | Screening Visit 1 | 135-330 U/L | 1120(H) |
| | | Visit 3 | 135-330 U/L | 962(H) |
| | | Visit 4 | 135-330 U/L | 381(H) |
| | | Visit 5 | 135-330 U/L | 276 |
| | | Visit 6 | 135-330 U/L | 234 |
| | | Visit 7 | 135-330 U/L | 265 |
| | | Visit 8 | 135-330 U/L | 280 |
| | | Visit 9 | 135-330 U/L | 234 |
| | | Visit 10 | 135-330 U/L | 284 |
| | | Visit 11 | 135-330 U/L | 252 |
| | | Visit 12 | 135-330 U/L | 244 |
| | | Visit 13 | 135-330 U/L | 303 |
| | | Tv1r | 135-330 U/L | 248 |
| | | Tv2r | 135-330 U/L | 241 |
| 410008103 | 51/F | Screening Visit 1 | 135-330 U/L | 1560(H) |
| | | Visit 3 | 135-330 U/L | 1160(H) |
| | | Unscheduled Visit 103.02 | 135-330 U/L | 496(H) |
| | | Visit 4 | 135-330 U/L | 305 |
| | | Visit 5 | 135-330 U/L | 271 |
| | | Visit 6 | 135-330 U/L | 236 |
| | | Visit 7 | 135-330 U/L | 257 |
| | | Visit 8 | 135-330 U/L | 223 |
| | | Visit 9 | 135-330 U/L | 200 |
| | | Visit 10 | 135-330 U/L | 260 |
| | | Visit 11 | 135-330 U/L | 245 |
| | | Visit 12 | 135-330 U/L | 260 |
| | | Visit 13 | 135-330 U/L | 220 |
| 458001102 | 30/F | Screening Visit 1 | 135-330 U/L | 3180(H) |
| | | Unscheduled Visit 102.01 | 135-330 U/L | 2760(H) |
| | | Visit 3 | 135-330 U/L | 2580(H) |
| | | Visit 4 | 135-330 U/L | 323 |
| | | Visit 5 | 135-330 U/L | 244 |
| | | Visit 6 | 135-330 U/L | 255 |
| | | Visit 7 | 135-330 U/L | 264 |
| | | Visit 8 | 135-330 U/L | 314 |
| | | Visit 9 | 135-330 U/L | 252 |
| | | Visit 10 | 135-330 U/L | 245 |
| | | Visit 11 | 135-330 U/L | 193 |
| | | Visit 12 | 135-330 U/L | 261 |
| | | Visit 13 | 135-330 U/L | 329 |
| | | Unscheduled Visit 113.02 | 135-330 U/L | 258 |
| 458002102 | 45/F | Screening Visit 1 | 135-330 U/L | 1271(H) |
| | | Visit 3 | 135-330 U/L | 1725(H) |
| | | Visit 4 | 135-330 U/L | 277 |
| | | Visit 5 | 135-330 U/L | 208 |
| | | Visit 6 | 135-330 U/L | 263 |
| | | Visit 6 | 135-330 U/L | 285 |
| | | Visit 7 | 135-330 U/L | 334(H) |
| | | Visit 8 | 135-330 U/L | 193 |
| | | Visit 9 | 135-330 U/L | 207 |
| | | Visit 10 | 135-330 U/L | 199 |
| | | Visit 11 | 135-330 U/L | 202 |
| | | Visit 12 | 135-330 U/L | 184 |
| | | Visit 13 | 135-330 U/L | 220 |
| | | Unscheduled Visit 214.01 | 135-330 U/L | 190 |
| 702001101 | 61/F | Screening Visit 1 | 135-330 U/L | 1530(H) |
| | | Visit 3 | 135-330 U/L | 1560(H) |
| | | Visit 4 | 135-330 U/L | 380(H) |
| | | Visit 5 | 135-330 U/L | 237 |
| | | Visit 6 | 135-330 U/L | 246 |
| | | Visit 7 | 135-330 U/L | 245 |
| | | Visit 8 | 135-330 U/L | 250 |
| | | Visit 9 | 135-330 U/L | 244 |
| | | Visit 10 | 135-330 U/L | 246 |
| | | Visit 11 | 135-330 U/L | 243 |
| | | Visit 12 | 135-330 U/L | 252 |
| 724001102 | 20/F | Screening Visit 1 | 135-330 U/L | 883(H) |
| | | Visit 3 | 135-330 U/L | 993(H) |
| | | Visit 4 | 135-330 U/L | 292 |
| | | Visit 5 | 135-330 U/L | 228 |
| | | Visit 6 | 135-330 U/L | 289 |
| | | Visit 7 | 135-330 U/L | 220 |
| | | Visit 8 | 135-330 U/L | 188 |
| 764002101 | 36/F | Screening Visit 1 | 135-330 U/L | 2580(H) |
| | | Visit 3 | 135-330 U/L | 2700(H) |
| | | Visit 4 | 135-330 U/L | 515(H) |
| | | Visit 5 | 135-330 U/L | 342(H) |
| | | Visit 6 | 135-330 U/L | 336(H) |
| | | Visit 7 | 135-330 U/L | 396(H) |
| | | Visit 8 | 135-330 U/L | 317 |
| 764002103 | 32/M | Screening Visit 1 | 135-281 U/L | 6045(H) |
| | | Visit 3 | 135-281 U/L | 5415(H) |
| | | Visit 4 | 135-281 U/L | 326(H) |
| | | Visit 5 | 135-281 U/L | 337(H) |
| | | Visit 6 | 135-281 U/L | 347(H) |
| | | Visit 7 | 135-281 U/L | 348(H) |
| 764004102 | 42/F | Screening Visit 1 | 135-330 U/L | 4230(H) |
| | | Visit 3 | 135-330 U/L | 2880(H) |
| | | Visit 4 | 135-330 U/L | 586(H) |

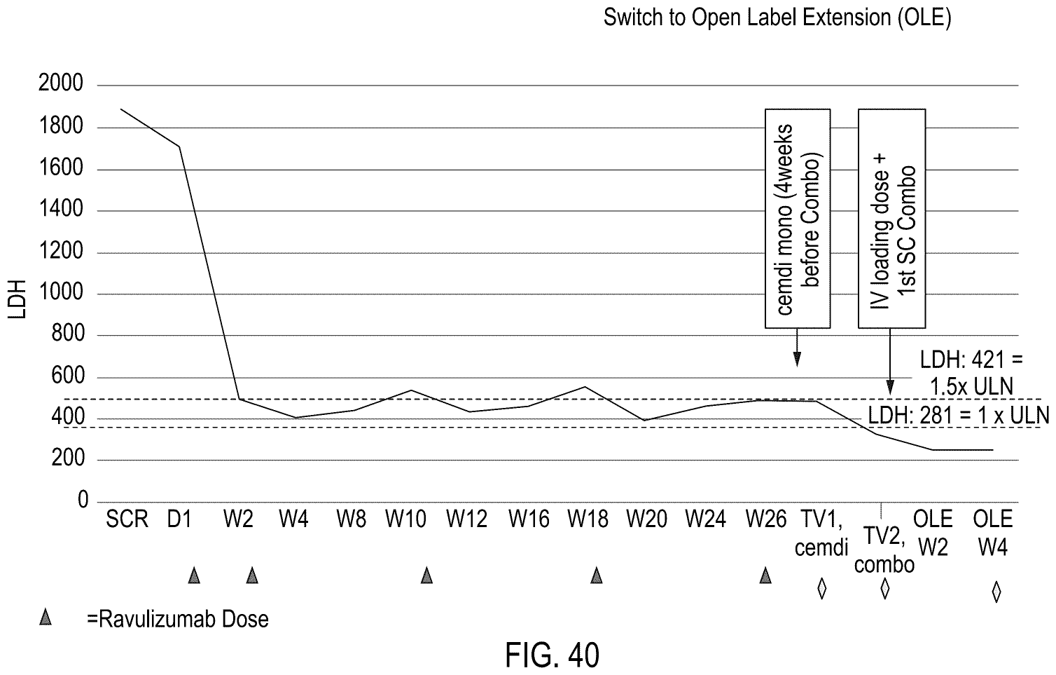

An inadequate responder in the Ravulizumab group (Subject ID. 158-001-101) normalized LDH after switching to the combo (first of 4 to cross over). See FIG. 40 wherein LDH levels over time before and after switch to combo are set forth. Prior to the switch from Ravulizumab to the combination, a saw-tooth-like pattern was observed. This pattern was not obvious after the switch.

As shown in FIG. 40, the patient (Subject ID. 158-001-101) who switched from Ravulizumab therapy received 200 mg SC Cemdisiran 4 weeks after the last dose of Ravulizumab, then, 4 weeks later, received an IV loading dose of 60 mg/kg Pozelimab and the first SC dose of combination (200 mg Cemdisiran and 400 mg Pozelimab). Another SC maintenance dose of the combination was given another 4 weeks later.

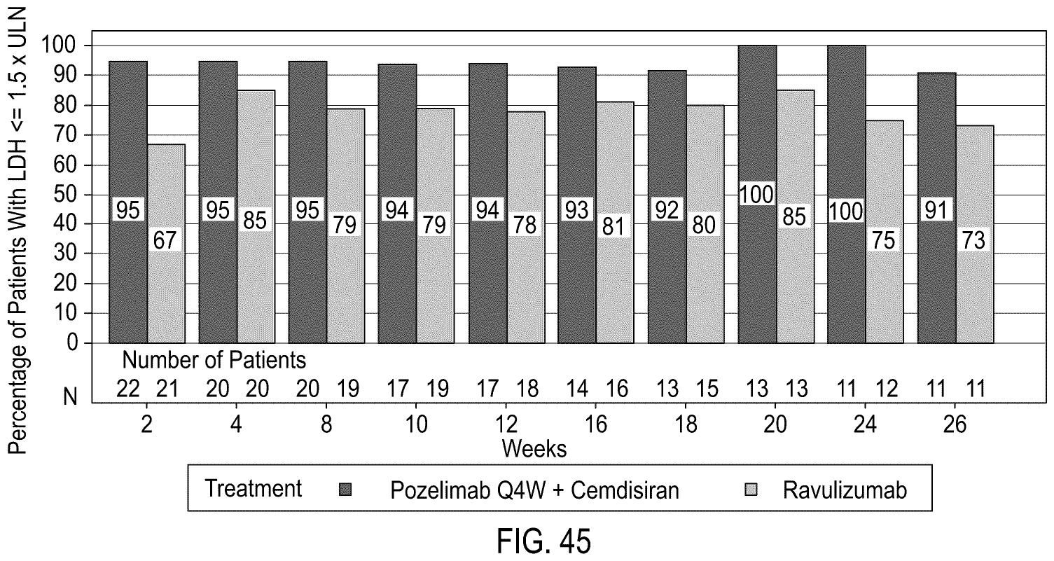

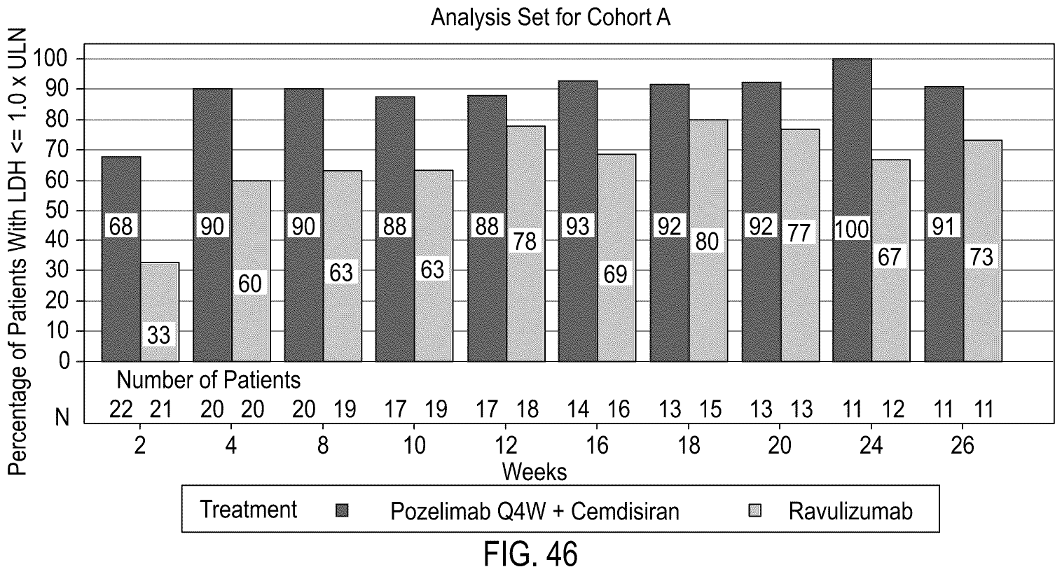

As set forth in Table 7-9 and 7-10, among Combo patients there was a higher proportion that achieved adequate control of hemolysis (i.e., achieved LDH≤1.5×ULN By Visit) and normalization of LDH (i.e., achieved LDH≤1×ULN By Visit) as compared to that of Ravulizumab patients. See FIG. 45 and FIG. 46.

TABLE 7-9
Percentage of Patients with LDH <= 1.5 ULN by Visit - Analysis
set for Cohort A (number of patients in parentheses)

| Week | Combo | Ravulizumab |
|---|---|---|
| 2 | 95 (22) | 67 (21) |
| 4 | 95 (20) | 85 (20) |
| 8 | 95 (20) | 79 (19) |
| 10 | 94 (17) | 79 (19) |
| 12 | 94 (17) | 78 (18) |
| 16 | 93 (14) | 81 (16) |
| 18 | 92 (13) | 80 (15) |
| 20 | 100 (13) | 85 (13) |
| 24 | 100 (11) | 75 (12) |
| 26 | 91 (11) | 73 (11) |

TABLE 7-10
Percentage of Patients with LDH <= 1.0 ULN by Visit - Analysis
Set for Cohort A (number of patients in parentheses)

| Week | Combo | Ravulizumab |
|---|---|---|
| 2 | 68 (22) | 33 (21) |
| 4 | 90 (20) | 60 (20) |
| 8 | 90 (20) | 63 (19) |
| 10 | 88 (17) | 63 (19) |
| 12 | 88 (17) | 78 (18) |
| 16 | 93 (14) | 69 (16) |
| 18 | 92 (13) | 80 (15) |
| 20 | 92 (13) | 77 (13) |
| 24 | 100 (11) | 67 (12) |
| 26 | 91 (11) | 73 (11) |

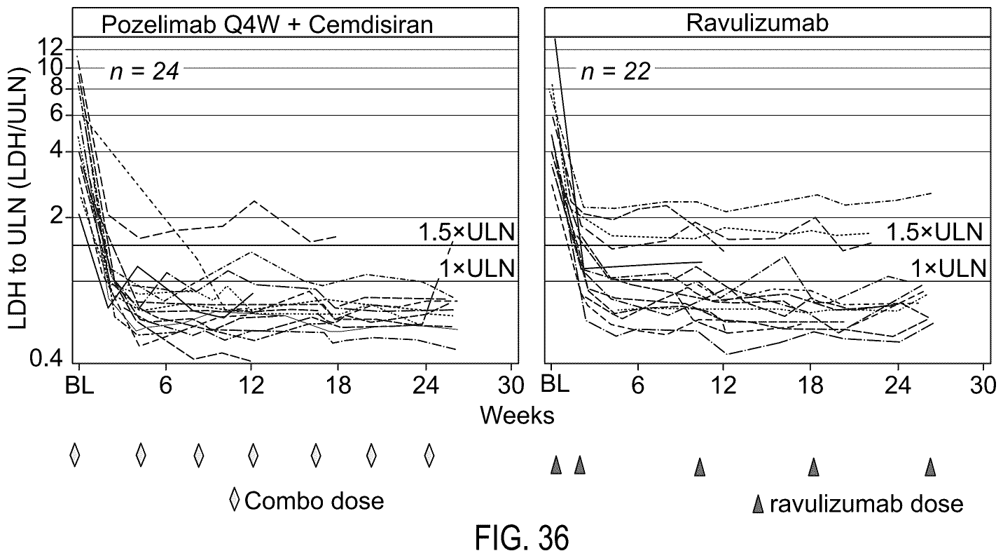

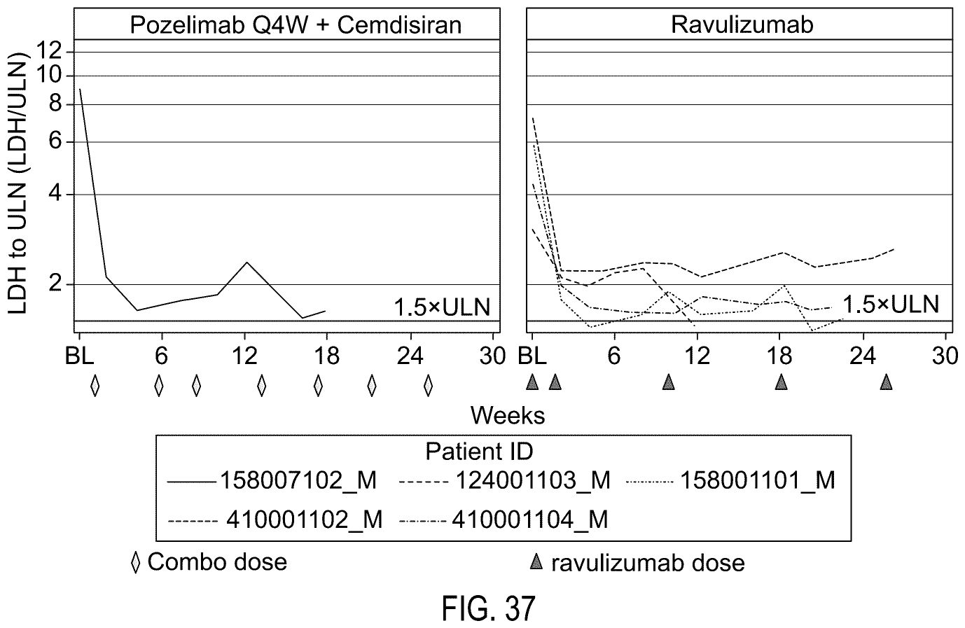

The majority of patients in both treatment groups (Combo and Ravulizumab) achieve control of LDH (below 1.5×ULN) by week 4 and maintained control through week 26 endpoint. See FIG. 36. Five patients failed to achieve adequate control of LDH by week 8—4 patients in Ravulizumab arm and patient in Combo arm. See FIG. 37. The Combo patients had a higher proportion of patients achieving LDH normalization (:51×ULN By Visit) compared to the Ravulizumab patients (Table 7-11).

TABLE 7-11
Clinical Laboratory Tests-LDH/ULN (Full Analysis Set)

| | | | | | |
|---|---|---|---|---|---|
| | | | | Ratio of | Ratio of |
| | | Analysis | Analysis | LDH to | LDH to |
| Subject ID | Visit | visit | value | ULN ≤1 | ULN ≤1.5 |

Pozelimab q4w + Cemdisiran

| | | | | | |
|---|---|---|---|---|---|
| 158003102 | Screening | | 2.17 | | |
| | Visit 1 | | | | |
| | Visit 3 | Baseline | 2.11 | N | N |
| | Visit 4 | Week 2 | 0.76 | Y | Y |
| | Visit 5 | Week 4 | 1.2 | N | Y |
| | Visit 6 | Week 8 | 0.74 | Y | Y |
| | Visit 7 | Week 10 | 0.68 | Y | Y |
| | Visit 8 | Week 12 | 0.9 | Y | Y |
| 158007102 | Screening | | 9.18 | | |
| | Visit 1 | | | | |
| | Visit 3 | Baseline | 8.97 | N | N |
| | Visit 4 | Week 2 | 2.12 | N | N |
| | Visit 5 | Week 4 | 1.62 | N | N |
| | Visit 6 | Week 8 | 1.78 | N | N |
| | Visit 7 | Week 10 | 1.83 | N | N |
| | Visit 8 | Week 12 | 2.36 | N | N |
| | Visit 9 | Week 16 | 1.54 | N | N |
| | Visit 10 | Week 18 | 1.63 | N | N |
| 380003101 | Screening | | 5 | | |
| | Visit 1 | | | | |
| | Visit 3 | Baseline | 5.14 | N | N |
| | Visit 4 | Week 2 | 0.99 | Y | Y |
| | Visit 5 | Week 4 | 0.88 | Y | Y |
| | Visit 6 | Week 8 | 0.93 | Y | Y |
| 410002101 | Screening | | 2.9 | | |
| | Visit 1 | | | | |
| | Visit 3 | Baseline | 3.06 | N | N |
| | Visit 4 | Week 2 | 0.9 | Y | Y |
| | Visit 5 | Week 4 | 0.81 | Y | Y |
| | Visit 6 | Week 8 | 0.79 | Y | Y |
| | Visit 7 | Week 10 | 0.75 | Y | Y |
| | Visit 8 | Week 12 | 0.77 | Y | Y |
| | Visit 9 | Week 16 | 0.78 | Y | Y |
| | Visit 10 | Week 18 | 0.76 | Y | Y |
| | Visit 11 | Week 20 | 0.85 | Y | Y |
| | Visit 12 | Week 24 | 0.86 | Y | Y |
| | Visit 13 | Week 26 | 0.85 | Y | Y |
| 410005102 | Screening | | 9.13 | | |
| | Visit 1 | | | | |
| | Visit 3 | Baseline | 10.68 | N | N |
| | Visit 4 | Week 2 | 0.98 | Y | Y |
| | Visit 5 | Week 4 | 0.68 | Y | Y |
| | Visit 6 | Week 8 | 0.57 | Y | Y |
| | Visit 7 | Week 10 | 0.65 | Y | Y |
| | Visit 8 | Week 12 | 0.78 | Y | Y |
| | Visit 9 | Week 16 | 0.65 | Y | Y |
| | Visit 10 | Week 18 | 0.66 | Y | Y |
| | Visit 11 | Week 20 | 0.68 | Y | Y |
| | Visit 12 | Week 24 | 0.63 | Y | Y |
| | Visit 13 | Week 26 | 0.6 | Y | Y |
| 410005105 | Screening | | 3.79 | | |
| | Visit 1 | | | | |
| | Visit 3 | Baseline | 4.15 | N | N |
| | Visit 4 | Week 2 | 0.89 | Y | Y |
| | Visit 5 | Week 4 | 0.56 | Y | Y |
| | Visit 6 | Week 8 | 0.59 | Y | Y |
| 410006101 | Screening | | 4.68 | | |
| | Visit 1 | | | | |
| | Visit 3 | Baseline | 4.79 | N | N |
| | Visit 4 | Week 2 | 0.7 | Y | Y |
| | Visit 5 | Week 4 | 0.59 | Y | Y |
| | Visit 6 | Week 8 | 0.68 | Y | Y |
| | Visit 7 | Week 10 | 0.56 | Y | Y |
| | Visit 8 | Week 12 | 0.54 | Y | Y |
| | Visit 9 | Week 16 | 0.64 | Y | Y |
| | Visit 10 | Week 18 | 0.52 | Y | Y |
| | Visit 11 | Week 20 | 0.56 | Y | Y |
| | Visit 12 | Week 24 | 0.54 | Y | Y |
| | Visit 13 | Week 26 | 0.5 | Y | Y |
| 410007101 | Screening | | 5.64 | | |
| | Visit 1 | | | | |
| | Visit 3 | Baseline | 5.28 | N | N |
| | Visit 4 | Week 2 | 1 | Y | Y |
| | Visit 5 | Week 4 | 0.72 | Y | Y |
| | Visit 6 | Week 8 | 0.77 | Y | Y |
| | Visit 6 | Week 8 | 0.77 | Y | Y |
| | Visit 7 | Week 10 | 0.81 | Y | Y |
| | Visit 8 | Week 12 | 0.71 | Y | Y |
| | Visit 9 | Week 16 | 0.7 | Y | Y |
| | Visit 10 | Week 18 | 0.71 | Y | Y |
| | Visit 11 | Week 20 | 0.72 | Y | Y |
| | Visit 12 | Week 24 | 0.83 | Y | Y |
| | Visit 13 | Week 26 | 0.82 | Y | Y |
| 410007102 | Screening | | 6.57 | | |
| | Visit 1 | | | | |
| | Visit 3 | Baseline | 10.73 | N | N |
| | Visit 4 | Week 2 | 1.07 | N | Y |
| | Visit 5 | Week 4 | 0.62 | Y | Y |
| | Visit 6 | Week 8 | 0.73 | Y | Y |
| | Visit 7 | Week 10 | 0.75 | Y | Y |
| | Visit 8 | Week 12 | 0.72 | Y | Y |
| | Visit 9 | Week 16 | 0.7 | Y | Y |
| | Visit 10 | Week 18 | 0.73 | Y | Y |
| | Visit 11 | Week 20 | 0.65 | Y | Y |
| | Visit 12 | Week 24 | 0.71 | Y | Y |
| | Visit 13 | Week 26 | 0.72 | Y | Y |
| 410008101 | Screening | | 3.38 | | |
| | Visit 1 | | | | |
| | Visit 3 | Baseline | 2.49 | N | N |
| | Visit 4 | Week 2 | 0.99 | Y | Y |
| | Visit 5 | Week 4 | 0.98 | Y | Y |
| | Visit 6 | Week 8 | 0.83 | Y | Y |
| | Visit 7 | Week 10 | 0.97 | Y | Y |
| | Visit 8 | Week 12 | 1.18 | N | Y |
| | Visit 8 | Week 12 | 1.4 | N | Y |
| | Visit 9 | Week 16 | 0.99 | Y | Y |
| | Visit 10 | Week 18 | 0.97 | Y | Y |
| | Visit 11 | Week 20 | 1.09 | N | Y |
| | Unscheduled | | 0.89 | | |
| | Visit 111.01 | | | | |
| | Visit 12 | Week 24 | 1 | Y | Y |
| | Visit 13 | Week 26 | 0.85 | Y | Y |
| 458001101 | Screening | | 7.63 | | |
| | Visit 1 | | | | |
| | Visit 3 | Baseline | 6.94 | N | N |
| | Visit 4 | Week 2 | 1.05 | N | Y |
| | Visit 5 | Week 4 | 0.7 | Y | Y |
| | Visit 6 | Week 8 | 0.68 | Y | Y |
| | Visit 7 | Week 10 | 0.99 | Y | Y |
| | Visit 8 | Week 12 | 0.72 | Y | Y |
| | Visit 9 | Week 16 | 0.84 | Y | Y |
| | Visit 10 | Week 18 | 0.84 | Y | Y |
| | Visit 11 | Week 20 | 0.77 | Y | Y |
| | Visit 12 | Week 24 | 0.74 | Y | Y |
| | Visit 13 | Week 26 | 0.85 | Y | Y |
| 458002101 | Screening | | 9.82 | | |
| | Visit 1 | | | | |
| | Visit 3 | Baseline | 8.43 | N | N |
| | Visit 4 | Week 2 | 0.89 | Y | Y |
| | Visit 5 | Week 4 | 0.74 | Y | Y |
| | Visit 6 | Week 8 | 1.11 | N | Y |
| | Visit 6 | Week 8 | 0.91 | Y | Y |
| | Visit 7 | Week 10 | 1.15 | N | Y |
| | Visit 8 | Week 12 | 0.98 | Y | Y |
| | Visit 9 | Week 16 | 0.94 | Y | Y |
| | Visit 10 | Week 18 | 0.72 | Y | Y |
| | Visit 11 | Week 20 | 0.74 | Y | Y |
| | Visit 12 | Week 24 | 0.75 | Y | Y |
| | Visit 13 | Week 26 | 0.75 | Y | Y |
| 458002103 | Screening | | 7.09 | | |
| | Visit 1 | | | | |
| | Unscheduled | Baseline | 9.41 | N | N |
| | Visit 101.01 | | | | |
| | Unscheduled | | 1.58 | | |
| | Visit 104.02 | | | | |
| | Visit 4 | Week 2 | 1.18 | N | Y |
| | Visit 5 | Week 4 | 0.67 | Y | Y |
| | Visit 6 | Week 8 | 0.44 | Y | Y |
| | Visit 7 | Week 10 | 0.48 | Y | Y |
| | Visit 8 | Week 12 | 0.43 | Y | Y |
| 458003101 | Screening | | 9.13 | | |
| | Visit 1 | | | | |
| | Unscheduled | | 11.85 | | |
| | Visit 102.01 | | | | |
| | Visit 3 | Baseline | 9.07 | N | N |
| | Visit 4 | Week 2 | 1.17 | N | Y |
| | Visit 5 | Week 4 | 0.74 | Y | Y |
| | Visit 6 | Week 8 | 0.62 | Y | Y |
| | Visit 7 | Week 10 | 0.71 | Y | Y |
| | Visit 8 | Week 12 | 0.69 | Y | Y |
| | Visit 9 | Week 16 | 0.91 | Y | Y |
| | Visit 10 | Week 18 | 0.67 | Y | Y |
| | Visit 11 | Week 20 | 0.78 | Y | Y |
| | Visit 12 | Week 24 | 0.63 | Y | Y |
| | Visit 13 | Week 26 | 0.63 | Y | Y |
| 458003102 | Screening | | 6.25 | | |
| | Visit 1 | | | | |
| | Visit 3 | Baseline | 7.63 | N | N |
| | Visit 4 | Week 2 | 0.82 | Y | Y |
| | Visit 5 | Week 4 | 0.64 | Y | Y |
| | Visit 6 | Week 8 | 0.62 | Y | Y |
| | Visit 7 | Week 10 | 0.6 | Y | Y |
| | Visit 8 | Week 12 | 0.61 | Y | Y |
| | Visit 9 | Week 16 | 0.7 | Y | Y |
| | Visit 10 | Week 18 | 0.63 | Y | Y |
| | Visit 11 | Week 20 | 0.62 | Y | Y |
| | Visit 12 | Week 24 | 0.64 | Y | Y |
| | Visit 13 | Week 26 | 1.6 | N | N |
| 616002101 | Screening | | 2.05 | | |
| | Visit 1 | | | | |
| | Unscheduled | | 2.64 | | |
| | Visit 102.01 | | | | |
| | Visit 3 | Baseline | 2.62 | N | N |
| | Visit 4 | Week 2 | 0.91 | Y | Y |
| | Visit 5 | Week 4 | 0.73 | Y | Y |
| | Visit 6 | Week 8 | 0.72 | Y | Y |
| | Visit 7 | Week 10 | 0.7 | Y | Y |
| | Visit 8 | Week 12 | 0.75 | Y | Y |
| 702001102 | Screening | | 9.02 | | |
| | Visit 1 | | | | |
| | Visit 3 | Baseline | 11.64 | N | N |
| | Visit 4 | Week 2 | 0.92 | Y | Y |
| | Visit 5 | Week 4 | 0.5 | Y | Y |
| | Visit 6 | Week 8 | 0.63 | Y | Y |
| | Visit 7 | Week 10 | 0.54 | Y | Y |
| | Visit 8 | Week 12 | 0.59 | Y | Y |
| | Visit 9 | Week 16 | 0.58 | Y | Y |
| | Visit 10 | Week 18 | 0.58 | Y | Y |
| | Visit 11 | Week 20 | 0.63 | Y | Y |
| 724001101 | Screening | | 3.65 | | |
| | Visit 1 | | | | |
| | Visit 3 | Baseline | 2.44 | N | N |
| | Visit 4 | Week 2 | 1.2 | N | Y |
| | Visit 5 | Week 4 | 0.95 | Y | Y |
| | Visit 6 | Week 8 | 0.9 | Y | Y |
| | Visit 7 | Week 10 | 0.75 | Y | Y |
| | Visit 8 | Week 12 | 0.77 | Y | Y |
| | Visit 9 | Week 16 | 0.75 | Y | Y |
| | Visit 10 | Week 18 | 0.62 | Y | Y |
| | Visit 11 | Week 20 | 0.79 | Y | Y |
| | Visit 12 | Week 24 | 0.72 | Y | Y |
| | Visit 13 | Week 26 | 0.7 | Y | Y |
| 724002101 | Screening | | 3.82 | | |
| | Visit 1 | | | | |
| | Visit 3 | Baseline | 3.69 | N | N |
| | Visit 4 | Week 2 | 0.97 | Y | Y |
| | Visit 5 | Week 4 | 0.75 | Y | Y |
| | Visit 6 | Week 8 | 0.75 | Y | Y |
| | Visit 8 | Week 12 | 0.69 | Y | Y |
| | Visit 9 | Week 16 | 0.8 | Y | Y |
| | Visit 11 | Week 20 | 0.74 | Y | Y |
| 764003102 | Screening | Baseline | 7.23 | N | N |
| | Visit 1 | | | | |
| 764004101 | Screening | | 8.86 | | |
| | Visit 1 | | | | |
| | Visit 3 | Baseline | 9.13 | N | N |
| | Visit 4 | Week 2 | 0.95 | Y | Y |
| 764005101 | Screening | | 7.73 | | |
| | Visit 1 | | | | |
| | Visit 3 | Baseline | 11.27 | N | N |
| | Visit 4 | Week 2 | 0.69 | Y | Y |
| 792001103 | Screening | | 3.58 | | |
| | Visit 1 | | | | |
| | Visit 3 | Baseline | 5.68 | N | N |
| | Visit 4 | Week 2 | 1.03 | N | Y |
| | Visit 5 | Week 4 | 0.79 | Y | Y |
| 840001102 | Screening | | 8 | | |
| | Visit 1 | | | | |
| | Visit 3 | Baseline | 6.5 | N | N |
| | Visit 6 | Week 8 | 1.17 | N | Y |
| | Visit 7 | Week 10 | 0.66 | Y | Y |

Ravulizumab

| | | | | | |
|---|---|---|---|---|---|
| 124001102 | Screening | | 8.33 | | |
| | Visit 1 | | | | |
| | Screening | | 3.61 | | |
| | Visit 1 | | | | |
| | Visit 3 | Baseline | 4.91 | N | N |
| | Visit 4 | Week 2 | 0.97 | Y | Y |
| | Visit 5 | Week 4 | 0.87 | Y | Y |
| | Visit 6 | Week 8 | 0.76 | Y | Y |
| | Visit 7 | Week 10 | 0.83 | Y | Y |
| | Visit 8 | Week 12 | 0.8 | Y | Y |
| | Visit 9 | Week 16 | 0.78 | Y | Y |
| | Visit 10 | Week 18 | 0.83 | Y | Y |
| 124001103 | Screening | | 4.91 | | |
| | Visit 1 | | | | |
| | Visit 3 | Baseline | 3.06 | N | N |
| | Visit 4 | Week 2 | 2.09 | N | N |
| | Visit 5 | Week 4 | 1.97 | N | N |
| | Visit 6 | Week 8 | 2.2 | N | N |
| | Visit 7 | Week 10 | 2.27 | N | N |
| | Visit 8 | Week 12 | 1.79 | N | N |
| | Visit 9 | Week 16 | 1.4 | N | Y |
| 124001105 | Screening | | 1.85 | | |
| | Visit 1 | | | | |
| | Screening | | 2.03 | | |
| | Visit 1 | | | | |
| | Visit 3 | Baseline | 1.83 | N | N |
| 158001101 | Screening | | 6.73 | | |
| | Visit 1 | | | | |
| | Visit 3 | Baseline | 6.09 | N | N |
| | Visit 4 | Week 2 | 1.79 | N | N |
| | Visit 5 | Week 4 | 1.45 | N | Y |
| | Visit 6 | Week 8 | 1.57 | N | N |
| | Visit 7 | Week 10 | 1.91 | N | N |
| | Visit 8 | Week 12 | 1.57 | N | N |
| | Visit 9 | Week 16 | 1.65 | N | N |
| | Visit 10 | Week 18 | 1.98 | N | N |
| | Visit 11 | Week 20 | 1.4 | N | Y |
| | Visit 12 | Week 24 | 1.63 | N | N |
| | Visit 13 | Week 26 | 1.74 | N | N |
| 158003101 | Screening | | 3.66 | | |
| | Visit 1 | | | | |
| | Visit 3 | Baseline | 3.72 | N | N |
| | Visit 4 | Week 2 | 0.88 | Y | Y |
| | Visit 5 | Week 4 | 0.64 | Y | Y |
| | Visit 6 | Week 8 | 0.58 | Y | Y |
| | Unscheduled | Week 10 | 0.69 | Y | Y |
| | Visit 107.01 | | | | |
| | Visit 8 | Week 12 | 0.68 | Y | Y |
| | Visit 9 | Week 16 | 0.61 | Y | Y |
| | Visit 10 | Week 18 | 0.67 | Y | Y |
| 158007101 | Screening | | 7.53 | | |
| | Visit 1 | | | | |
| | Visit 3 | Baseline | 7.42 | N | N |
| | Visit 4 | Week 2 | 1.68 | N | N |
| | Visit 5 | Week 4 | 1.04 | N | Y |
| | Visit 6 | Week 8 | 1.09 | N | Y |
| | Visit 7 | Week 10 | 0.88 | Y | Y |
| | Visit 8 | Week 12 | 0.86 | Y | Y |
| | Visit 9 | Week 16 | 1.34 | N | Y |
| | Visit 10 | Week 18 | 0.8 | Y | Y |
| | Visit 11 | Week 20 | 0.85 | Y | Y |
| | Visit 12 | Week 24 | 1.06 | N | Y |
| | Visit 13 | Week 26 | 0.99 | Y | Y |
| 392004101 | Screening | | 5.47 | | |
| | Visit 1 | | | | |
| | Visit 3 | Baseline | 4.4 | N | N |
| | Visit 4 | Week 2 | 1.2 | N | Y |
| | Visit 5 | Week 4 | 0.98 | Y | Y |
| 410001102 | Screening | | 4.91 | | |
| | Visit 1 | | | | |
| | Visit 3 | Baseline | 7.31 | N | N |
| | Visit 4 | Week 2 | 2.22 | N | N |
| | Visit 5 | Week 4 | 2.2 | N | N |
| | Visit 6 | Week 8 | 2.36 | N | N |
| | Visit 7 | Week 10 | 2.35 | N | N |
| | Visit 8 | Week 12 | 2.11 | N | N |
| | Visit 9 | Week 16 | 2.43 | N | N |
| | Visit 10 | Week 18 | 2.55 | N | N |
| | Visit 11 | Week 20 | 2.28 | N | N |
| | Visit 12 | Week 24 | 2.42 | N | N |
| | Visit 13 | Week 26 | 2.62 | N | N |
| 410001104 | Screening | | 4.46 | | |
| | Visit 1 | | | | |
| | Screening | | 4.79 | | |
| | Visit 1 | | | | |
| | Visit 3 | Baseline | 4.46 | N | N |
| | Visit 4 | Week 2 | 1.96 | N | N |
| | Visit 5 | Week 4 | 1.65 | N | N |
| | Visit 6 | Week 8 | 1.6 | N | N |
| | Visit 7 | Week 10 | 1.59 | N | N |
| | Visit 8 | Week 12 | 1.81 | N | N |
| | Visit 9 | Week 16 | 1.71 | N | N |
| | Visit 10 | Week 18 | 1.75 | N | N |
| | Visit 11 | Week 20 | 1.64 | N | N |
| | Visit 12 | Week 24 | 1.73 | N | N |
| | Visit 13 | Week 26 | 1.76 | N | N |
| 410003101 | Screening | | 7.15 | | |
| | Visit 1 | | | | |
| | Visit 3 | Baseline | 7.21 | N | N |
| | Visit 4 | Week 2 | 0.67 | Y | Y |
| | Visit 5 | Week 4 | 0.56 | Y | Y |
| | Visit 6 | Week 8 | 0.6 | Y | Y |
| | Visit 7 | Week 10 | 0.6 | Y | Y |
| | Visit 8 | Week 12 | 0.59 | Y | Y |
| | Visit 8 | Week 12 | 0.46 | Y | Y |
| | Visit 9 | Week 16 | 0.53 | Y | Y |
| | Visit 10 | Week 18 | 0.59 | Y | Y |
| | Visit 11 | Week 20 | 0.55 | Y | Y |
| | Visit 12 | Week 24 | 0.54 | Y | Y |
| | Visit 13 | Week 26 | 0.65 | Y | Y |
| 410004101 | Screening | | 2.43 | | |
| | Visit 1 | | | | |
| | Visit 3 | Baseline | 4 | N | N |
| | Visit 4 | Week 2 | 1.11 | N | Y |
| | Visit 5 | Week 4 | 0.97 | Y | Y |
| | Visit 6 | Week 8 | 0.9 | Y | Y |
| | Visit 7 | Week 10 | 0.84 | Y | Y |
| | Visit 8 | Week 12 | 0.8 | Y | Y |
| | Visit 9 | Week 16 | 0.89 | Y | Y |
| | Visit 10 | Week 18 | 0.84 | Y | Y |
| | Visit 11 | Week 20 | 0.8 | Y | Y |
| | Visit 12 | Week 24 | 0.81 | Y | Y |
| | Visit 13 | Week 26 | 0.89 | Y | Y |
| 410005101 | Screening | | 7.14 | | |
| | Visit 1 | | | | |
| | Visit 3 | Baseline | 6.86 | N | N |
| | Visit 4 | Week 2 | 1.21 | N | Y |
| | Visit 5 | Week 4 | 1.01 | N | Y |
| | Visit 6 | Week 8 | 0.99 | Y | Y |
| | Visit 7 | Week 10 | 0.99 | Y | Y |
| | Visit 8 | Week 12 | 0.85 | Y | Y |
| | Visit 9 | Week 16 | 0.94 | Y | Y |
| | Visit 10 | Week 18 | 0.92 | Y | Y |
| | Visit 11 | Week 20 | 0.78 | Y | Y |
| | Visit 12 | Week 24 | 0.78 | Y | Y |
| | Visit 13 | Week 26 | 0.82 | Y | Y |
| 410005103 | Screening | | 6.55 | | |
| | Visit 1 | | | | |
| | Visit 3 | Baseline | 5.1 | N | N |
| | Visit 4 | Week 2 | 1 | N | Y |
| | Visit 5 | Week 4 | 0.85 | Y | Y |
| | Visit 6 | Week 8 | 0.81 | Y | Y |
| | Visit 7 | Week 10 | 0.78 | Y | Y |
| | Visit 8 | Week 12 | 0.66 | Y | Y |
| | Visit 9 | Week 16 | 0.65 | Y | Y |
| | Visit 10 | Week 18 | 0.65 | Y | Y |
| | Visit 11 | Week 20 | 0.66 | Y | Y |
| 410008102 | Screening | | 3.39 | | |
| | Visit 1 | | | | |
| | Visit 3 | Baseline | 2.92 | N | N |
| | Visit 4 | Week 2 | 1.15 | N | Y |
| | Visit 5 | Week 4 | 0.84 | Y | Y |
| | Visit 6 | Week 8 | 0.71 | Y | Y |
| | Visit 7 | Week 10 | 0.8 | Y | Y |
| | Visit 8 | Week 12 | 0.85 | Y | Y |
| | Visit 9 | Week 16 | 0.71 | Y | Y |
| | Visit 10 | Week 18 | 0.86 | Y | Y |
| | Visit 11 | Week 20 | 0.76 | Y | Y |
| | Visit 12 | Week 24 | 0.74 | Y | Y |
| | Visit 13 | Week 26 | 0.92 | Y | Y |
| 410008103 | Screening | | 4.73 | | |
| | Visit 1 | | | | |
| | Visit 3 | Baseline | 3.52 | N | N |
| | Unscheduled | | 1.5 | | |
| | Visit 103.02 | | | | |
| | Visit 4 | Week 2 | 0.92 | Y | Y |
| | Visit 5 | Week 4 | 0.82 | Y | Y |
| | Visit 6 | Week 8 | 0.72 | Y | Y |
| | Visit 7 | Week 10 | 0.78 | Y | Y |
| | Visit 8 | Week 12 | 0.68 | Y | Y |
| | Visit 9 | Week 16 | 0.61 | Y | Y |
| | Visit 10 | Week 18 | 0.79 | Y | Y |
| | Visit 11 | Week 20 | 0.74 | Y | Y |
| | Visit 12 | Week 24 | 0.79 | Y | Y |
| | Visit 13 | Week 26 | 0.67 | Y | Y |
| 458001102 | Screening | | 9.64 | | |
| | Visit 1 | | | | |
| | Unscheduled | | 8.36 | | |
| | Visit 102.01 | | | | |
| | Visit 3 | Baseline | 7.82 | N | N |
| | Visit 4 | Week 2 | 0.98 | Y | Y |
| | Visit 5 | Week 4 | 0.74 | Y | Y |
| | Visit 6 | Week 8 | 0.77 | Y | Y |
| | Visit 7 | Week 10 | 0.8 | Y | Y |
| | Visit 8 | Week 12 | 0.95 | Y | Y |
| | Visit 9 | Week 16 | 0.76 | Y | Y |
| | Visit 10 | Week 18 | 0.74 | Y | Y |
| | Visit 11 | Week 20 | 0.58 | Y | Y |
| | Visit 12 | Week 24 | 0.79 | Y | Y |
| | Visit 13 | Week 26 | 1 | Y | Y |
| 458002102 | Screening | | 3.85 | | |
| | Visit 1 | | | | |
| | Visit 3 | Baseline | 5.23 | N | N |
| | Visit 4 | Week 2 | 0.84 | Y | Y |
| | Visit 5 | Week 4 | 0.63 | Y | Y |
| | Visit 6 | Week 8 | 0.8 | Y | Y |
| | Visit 6 | Week 8 | 0.86 | Y | Y |
| | Visit 7 | Week 10 | 1.01 | N | Y |
| | Visit 8 | Week 12 | 0.58 | Y | Y |
| | Visit 9 | Week 16 | 0.63 | Y | Y |
| | Visit 10 | Week 18 | 0.6 | Y | Y |
| | Visit 11 | Week 20 | 0.61 | Y | Y |
| | Visit 12 | Week 24 | 0.56 | Y | Y |
| | Visit 13 | Week 26 | 0.67 | Y | Y |
| 702001101 | Screening | | 4.64 | | |
| | Visit 1 | | | | |
| | Visit 3 | Baseline | 4.73 | N | N |
| | Visit 4 | Week 2 | 1.15 | N | Y |
| | Visit 5 | Week 4 | 0.72 | Y | Y |
| | Visit 6 | Week 8 | 0.75 | Y | Y |
| | Visit 7 | Week 10 | 0.74 | Y | Y |
| | Visit 8 | Week 12 | 0.76 | Y | Y |
| | Visit 9 | Week 16 | 0.74 | Y | Y |
| | Visit 10 | Week 18 | 0.75 | Y | Y |
| | Visit 11 | Week 20 | 0.74 | Y | Y |
| | Visit 12 | Week 24 | 0.76 | Y | Y |
| 724001102 | Screening | | 2.68 | | |
| | Visit 1 | | | | |
| | Visit 3 | Baseline | 3.01 | N | N |
| | Visit 4 | Week 2 | 0.88 | Y | Y |
| | Visit 5 | Week 4 | 0.69 | Y | Y |
| | Visit 6 | Week 8 | 0.88 | Y | Y |
| | Visit 7 | Week 10 | 0.67 | Y | Y |
| | Visit 8 | Week 12 | 0.57 | Y | Y |
| 764002101 | Screening | | 7.82 | | |
| | Visit 1 | | | | |
| | Visit 3 | Baseline | 8.18 | N | N |
| | Visit 4 | Week 2 | 1.56 | N | N |
| | Visit 5 | Week 4 | 1.04 | N | Y |
| | Visit 6 | Week 8 | 1.02 | N | Y |
| | Visit 7 | Week 10 | 1.2 | N | Y |
| | Visit 8 | Week 12 | 0.96 | Y | Y |
| 764002103 | Screening | | 21.51 | | |
| | Visit 1 | | | | |
| | Visit 3 | Baseline | 19.27 | N | N |
| | Visit 4 | Week 2 | 1.16 | N | Y |
| | Visit 5 | Week 4 | 1.2 | N | Y |
| | Visit 6 | Week 8 | 1.23 | N | Y |
| | Visit 7 | Week 10 | 1.24 | N | Y |
| 764004102 | Screening | | 12.82 | | |
| | Visit 1 | | | | |
| | Visit 3 | Baseline | 8.73 | N | N |
| | Visit 4 | Week 2 | 1.78 | N | N |

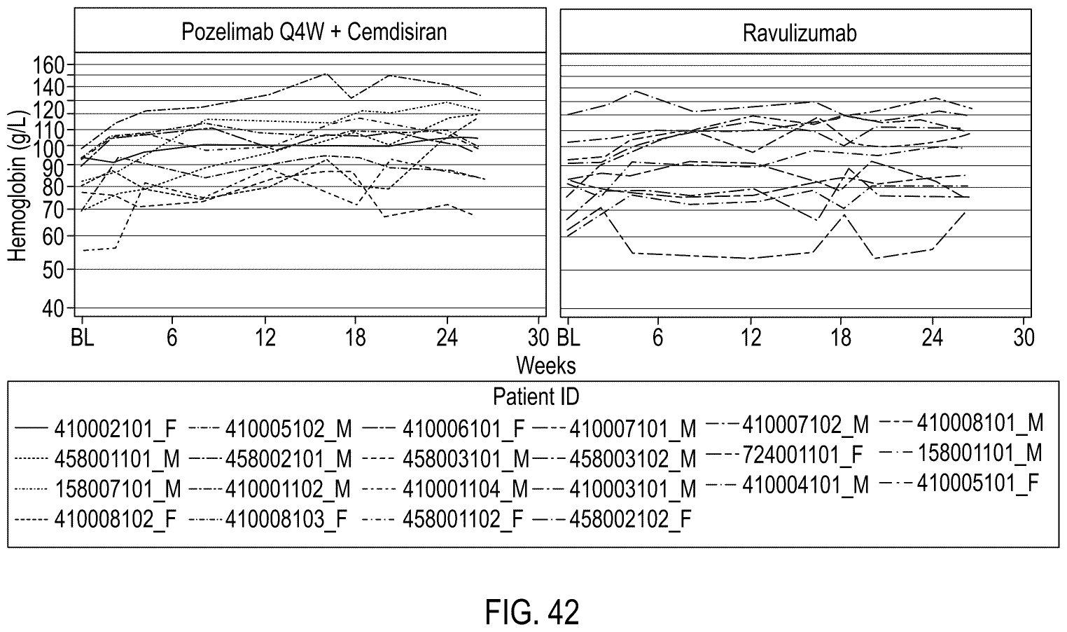

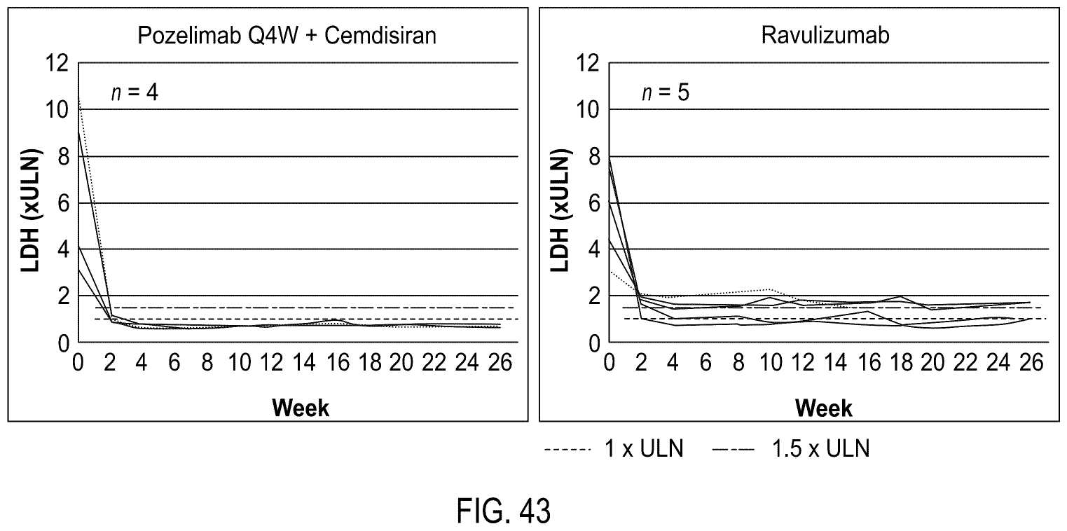

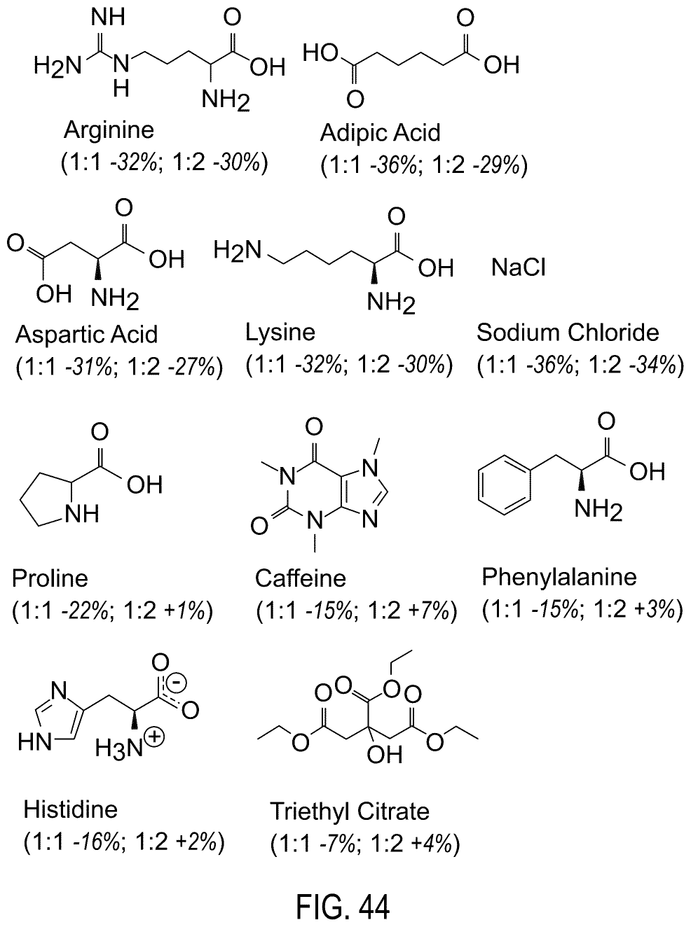

Hemoglobin (Hb) was similar across treatment groups among week 26 completers. A summary of change from baseline of hemoglobin measurements over 26 weeks is set forth in Table 7-12. Individual hemoglobin measurements per visit are set forth in Table 7-13. See also FIG. 42 which summarizes hemoglobin values over time among Combo patients and Ravulizumab patients.

TABLE 7-12
Summary of Changes in Hemoglobin over 26 Weeks

| | Baseline | Baseline mean | | |
|---|---|---|---|---|
| | mean Hb* | Hb\hat{ } (week 26 | Week | Change from |
| | (all patients) | completers) | 26 Hb\hat{ } | baseline |
| Combo | 84.6 | 82.1 | 102.5 | +20.5 |
| Ravulizumab | 87.9 | 83.4 | 96.2 | +12.8 |

Hb = hemoglobin;
reference range (g/L): 110 to 155 female, 125 to 170 male
n = 24 combo, 22 ravulizumab;
\hat{ }n = 11 combo and ravulizumab

TABLE 7-13
Clinical Laboratory Test-Hemoglobin (Full Analysis Set)

| | | | | |
|---|---|---|---|---|
| | | | | Result (g/L) |
| | | | Reference | (Out of Range |
| Subject ID | Age/Sex | Visit | Range | Flag |

Pozelimab q4w + Cemdisiran

| | | | | |
|---|---|---|---|---|
| 158003102 | 39/F | Screening Visit 1 | 110-155 g/L | 124 |
| | | Visit 3 | 110-155 g/L | 130 |
| | | Visit 4 | 110-155 g/L | 140 |
| | | Visit 5 | 110-155 g/L | 130 |
| | | Visit 6 | 110-155 g/L | 130 |
| | | Visit 8 | 110-155 g/L | 131 |
| 158007102 | 66/M | Screening Visit 1 | 125-170 g/L | 74(L) |
| | | Visit 3 | 125-170 g/L | 53(L) |
| | | Visit 4 | 125-170 g/L | 71(L) |
| | | Visit 5 | 125-170 g/L | 65(L) |
| | | Visit 6 | 125-170 g/L | 61(L) |
| | | Visit 8 | 125-170 g/L | 47(L) |
| | | Visit 9 | 125-170 g/L | 79(L) |
| | | Visit 10 | 125-170 g/L | 93(L) |
| 380003101 | 40/F | Screening Visit 1 | 110-155 g/L | 76(L) |
| | | Visit 3 | 110-155 g/L | 88(L) |
| | | Visit 4 | 110-155 g/L | 90(L) |
| | | Visit 5 | 110-155 g/L | 86(L) |
| | | Visit 6 | 110-155 g/L | 79(L) |
| 410002101 | 65/F | Screening Visit 1 | 110-155 g/L | 104(L) |
| | | Visit 3 | 110-155 g/L | 94(L) |
| | | Visit 4 | 110-155 g/L | 91(L) |
| | | Visit 5 | 110-155 g/L | 96(L) |
| | | Visit 6 | 110-155 g/L | 101(L) |
| | Visit 8 | 2022-12- | 100(L) | CENTRAL LAB |
| | | 23T09: 30(86) | | |
| 410002101 | 65/F | Visit 9 | 110-155 g/L | 101(L) |
| | | Visit 10 | | |
| | | Visit 11 | 110-155 g/L | 100(L) |
| | | Visit 12 | 110-155 g/L | 105(L) |
| | | Visit 13 | 110-155 g/L | 105(L) |
| 410005102 | 61/M | Screening Visit 1 | 125-170 g/L | 75(L) |
| | | Visit 3 | 125-170 g/L | 69(L) |
| | | Visit 4 | 125-170 g/L | 94(L) |
| | | Visit 5 | 125-170 g/L | 90(L) |
| | | Visit 6 | 125-170 g/L | 84(L) |
| | | Visit 8 | 125-170 g/L | 90(L) |
| | | Visit 9 | 125-170 g/L | 95(L) |
| | | Visit 10 | 125-170 g/L | 94(L) |
| | | Visit 11 | 125-170 g/L | 89(L) |
| | | Visit 12 | 125-170 g/L | 87(L) |
| | | Visit 13 | 125-170 g/L | 83(L) |
| 410005105 | 39/M | Screening Visit 1 | 125-170 g/L | 109(L) |
| | | Visit 3 | 125-170 g/L | 110(L) |
| | | Visit 4 | 125-170 g/L | 115(L) |
| | | Visit 5 | 125-170 g/L | 119(L) |
| | | Visit 6 | 125-170 g/L | 125 |
| 410006101 | 36/F | Screening Visit 1 | 110-155 g/L | 96(L) |
| | | Visit 3 | 110-155 g/L | 99(L) |
| | | Visit 4 | 110-155 g/L | 114 |
| | | Visit 5 | 110-155 g/L | 122 |
| 410006101 | 36/F | Visit 6 | 110-155 g/L | 125 |
| | | Visit 8 | 110-155 g/L | 134 |
| | | Visit 9 | 110-155 g/L | 152 |
| | | Visit 10 | 110-155 g/L | 132 |
| | | Visit 11 | 110-155 g/L | 150 |
| | | Visit 12 | 110-155 g/L | 141 |
| | | Visit 13 | 110-155 g/L | 133 |
| 410007101 | 40/M | Screening Visit 1 | 125-170 g/L | 77(L) |
| | | Visit 3 | 125-170 g/L | 93(L) |
| | | Visit 4 | 125-170 g/L | 105(L) |
| | | Visit 5 | 125-170 g/L | 107(L) |
| | | Visit 6 | 125-170 g/L | 98(L) |
| | | Visit 8 | 125-170 g/L | 100(L) |
| | | Visit 9 | 125-170 g/L | 113(L) |
| | | Visit 10 | 125-170 g/L | 117(L) |
| | | Visit 11 | | |
| | | Unscheduled Visit | 125-170 g/L | 103(L) |
| | | 111.01 | | |
| | | Visit 12 | 125-170 g/L | 107(L) |
| | | Visit 13 | 125-170 g/L | 98(L) |
| 410007102 | 58/M | Screening Visit 1 | 125-170 g/L | 79(L) |
| | | Visit 3 | 125-170 g/L | 90(L) |
| | | Visit 4 | 125-170 g/L | 106(L) |
| | | Visit 5 | | |
| | | Unscheduled Visit | 125-170 g/L | 93(L) |
| | | 105.01 | | |
| | | Visit 6 | 125-170 g/L | 111(L) |
| | | Visit 8 | 125-170 g/L | 98(L) |
| 410007102 | 58/M | Visit 9 | 125-170 g/L | 103(L) |
| | | Visit 10 | 125-170 g/L | 109(L) |
| | | Visit 11 | 125-170 g/L | 109(L) |
| | | Visit 12 | 125-170 g/L | 101(L) |
| | | Visit 13 | 125-170 g/L | 97(L) |
| 410008101 | 73/M | Screening Visit 1 | 125-170 g/L | 79(L) |
| | | Unscheduled Visit | 125-170 g/L | 93(L) |
| | | 102.01 | | |
| | | Visit 3 | 125-170 g/L | 77(L) |
| | | Visit 4 | 125-170 g/L | 76(L) |
| | | Visit 5 | 125-170 g/L | 71(L) |
| | | Visit 7 | 125-170 g/L | 73(L) |
| | | Unscheduled Visit | 125-170 g/L | 72(L) |
| | | 108.01 | | |
| | | Visit 8 | 125-170 g/L | 82(L) |
| | | Visit 9 | 125-170 g/L | 87(L) |
| | | Visit 10 | 125-170 g/L | 87(L) |
| | | Visit 11 | 125-170 g/L | 67(L) |
| | | Unscheduled Visit | 125-170 g/L | 78(L) |
| | | 111.01 | | |
| | | Visit 12 | 125-170 g/L | 72(L) |
| | | Visit 13 | 125-170 g/L | 67(L) |
| 458001101 | 44/M | Screening Visit 1 | 125-170 g/L | 47(L) |
| | | Visit 3 | 125-170 g/L | 55(L) |
| | | Visit 4 | 125-170 g/L | 56(L) |
| | | Visit 5 | 125-170 g/L | 81(L) |
| | | Visit 6 | 125-170 g/L | 75(L) |
| | | Visit 8 | 125-170 g/L | 88(L) |
| | | Visit 9 | 125-170 g/L | 75(L) |
| 458001101 | 44/M | Visit 10 | 125-170 g/L | 71(L) |
| | | Visit 11 | 125-170 g/L | 93(L) |
| | | Visit 12 | 125-170 g/L | 86(L) |
| | | Visit 13 | 125-170 g/L | 83(L) |
| 458002101 | 46/M | Screening Visit 1 | 125-170 g/L | 69(L) |
| | | Visit 3 | 125-170 g/L | 82(L) |
| | | Visit 4 | 125-170 g/L | 86(L) |
| | | Visit 5 | 125-170 g/L | 95(L) |
| | | Visit 6 | 125-170 g/L | 116(L) |
| | | Visit 8 | | |
| | | Unscheduled Visit | 125-170 g/L | 117(L) |
| | | 108.01 | | |
| | | Visit 9 | 125-170 g/L | 114(L) |
| | | Visit 10 | 125-170 g/L | 122(L) |
| | | Visit 11 | 125-170 g/L | 121(L) |
| | | Visit 12 | 125-170 g/L | 128 |
| | | Visit 13 | 125-170 g/L | 123(L) |
| | | Follow-up 1 | 125-170 g/L | 129 |
| 458002103 | 43/F | Screening Visit 1 | 110-155 g/L | 118 |
| | | Visit 3 | 110-155 g/L | 97(L) |
| | | Visit 4 | 110-155 g/L | 114 |
| | | Visit 5 | 110-155 g/L | 113 |
| | | Visit 6 | 110-155 g/L | 124 |
| | | Visit 8 | 110-155 g/L | 128 |
| 458003101 | 39/M | Screening Visit 1 | 125-170 g/L | 107(L) |
| | | Visit 3 | 125-170 g/L | 94(L) |
| 458003101 | 39/M | Visit 4 | 125-170 g/L | 106(L) |
| | | Visit 5 | 125-170 g/L | 107(L) |
| | | Visit 6 | 125-170 g/L | 114(L) |
| | | Visit 8 | 125-170 g/L | 108(L) |
| | | Visit 9 | 125-170 g/L | 106(L) |
| | | Visit 10 | 125-170 g/L | 109(L) |
| | | Visit 11 | 125-170 g/L | 101(L) |
| | | Visit 12 | 125-170 g/L | 117(L) |
| | | Visit 13 | 125-170 g/L | 120(L) |
| 458003102 | 29/M | Screening Visit 1 | 125-170 g/L | 68(L) |
| | | Visit 3 | 125-170 g/L | 70(L) |
| | | Visit 4 | 125-170 g/L | 76(L) |
| | | Visit 5 | 125-170 g/L | 79(L) |
| | | Visit 6 | 125-170 g/L | 89(L) |
| | | Visit 8 | 125-170 g/L | 98(L) |
| | | Visit 9 | 125-170 g/L | 107(L) |
| | | Visit 10 | 125-170 g/L | 106(L) |
| | | Visit 11 | 125-170 g/L | 108(L) |
| | | Visit 12 | 125-170 g/L | 109(L) |
| | | Visit 13 | 125-170 g/L | 100(L) |
| 616002101 | 21/F | Screening Visit 1 | 110-155 g/L | 81(L) |
| | | Unscheduled Visit | 110-155 g/L | 85(L) |
| | | 102.01 | | |
| | | Visit 3 | 110-155 g/L | 82(L) |
| | | Visit 4 | 110-155 g/L | 83(L) |
| | | Visit 5 | 110-155 g/L | 87(L) |
| | | Visit 6 | 110-155 g/L | 86(L) |
| 616002101 | 21/F | Visit 8 | 110-155 g/L | 95(L) |
| 702001102 | 47/M | Screening Visit 1 | 125-170 g/L | 81(L) |
| | | Visit 3 | 125-170 g/L | 68(L) |
| | | Visit 4 | 125-170 g/L | 108(L) |
| | | Visit 5 | 125-170 g/L | 105(L) |
| | | Visit 6 | 125-170 g/L | 94(L) |
| | | Visit 8 | 125-170 g/L | 97(L) |
| | | Visit 9 | 125-170 g/L | 102(L) |
| | | Visit 10 | 125-170 g/L | 97(L) |
| | | Visit 11 | 125-170 g/L | 104(L) |
| 724001101 | 48/F | Screening Visit 1 | 110-155 g/L | 102(L) |
| | | Visit 3 | 110-155 g/L | 80(L) |
| | | Visit 4 | 110-155 g/L | 87(L) |
| | | Visit 5 | 110-155 g/L | 78(L) |
| | | Visit 6 | 110-155 g/L | 74(L) |
| | | Visit 8 | 110-155 g/L | 79(L) |
| | | Visit 9 | 110-155 g/L | 93(L) |
| | | Visit 10 | 110-155 g/L | 80(L) |
| | | Visit 11 | 110-155 g/L | 79(L) |
| | | Visit 12 | 110-155 g/L | 105(L) |
| | | Visit 13 | 110-155 g/L | 119 |
| 724002101 | 87/F | Screening Visit 1 | 110-155 g/L | 112 |
| | | Visit 3 | 110-155 g/L | 105(L) |
| | | Visit 4 | 110-155 g/L | 105(L) |
| | | Visit 5 | 110-155 g/L | 98(L) |
| 724002101 | 87/F | Visit 6 | 110-155 g/L | 105(L) |
| | | Visit 8 | 110-155 g/L | 105(L) |
| | | Visit 9 | 110-155 g/L | 116 |
| | | Visit 11 | 110-155 g/L | 110 |
| 764003102 | 49/F | Screening Visit 1 | 110-155 g/L | 83(L) |
| 764004101 | 56/M | Screening Visit 1 | 125-170 g/L | 74(L) |
| | | Visit 3 | 125-170 g/L | 65(L) |
| | | Visit 4 | 125-170 g/L | 70(L) |
| 764005101 | 50/F | Screening Visit 1 | 110-155 g/L | 103(L) |
| | | Visit 3 | 110-155 g/L | 89(L) |
| | | Visit 4 | 110-155 g/L | 86(L) |
| 792001103 | 21/F | Screening Visit 1 | 110-155 g/L | 82(L) |
| | | Unscheduled Visit | 110-155 g/L | 88(L) |
| | | 101.01 | | |
| | | Unscheduled Visit | 110-155 g/L | 91(L) |
| | | 102.01 | | |
| | | Visit 3 | 110-155 g/L | 68(L) |
| | | Visit 4 | 110-155 g/L | 85(L) |
| | | Visit 5 | 110-155 g/L | 89(L) |
| 840001102 | 23/F | Screening Visit 1 | 110-155 g/L | 105(L) |
| | | Visit 3 | 110-155 g/L | 89(L) |
| | | Visit 5 | | |
| | | Visit 6 | 110-155 g/L | 96(L) |

Ravulizumab

| | | | | |
|---|---|---|---|---|
| 124001102 | 58/M | Screening Visit 1 | 125-170 g/L | 107(L) |
| | | Screening Visit 1 | | |
| | | Unscheduled Visit | 125-170 g/L | 131 |
| | | 102.01 | | |
| | | Visit 3 | 125-170 g/L | 124(L) |
| | | Visit 4 | 125-170 g/L | 124(L) |
| | | Visit 5 | 125-170 g/L | 139 |
| | | Visit 7 | | |
| | | Visit 9 | 125-170 g/L | 121(L) |
| | | Visit 10 | | |
| 124001103 | 21/M | Screening Visit 1 | 125-170 g/L | 69(L) |
| | | Visit 3 | 125-170 g/L | 71(L) |
| | | Visit 4 | 125-170 g/L | 80(L) |
| | | Visit 5 | 125-170 g/L | 67(L) |
| | | Visit 7 | | |
| | | Unscheduled Visit | 125-170 g/L | 75(L) |
| | | 108.01 | | |
| | | Visit 9 | 125-170 g/L | 78(L) |
| 124001105 | 36/M | Screening Visit 1 | 125-170 g/L | 138 |
| | | Screening Visit 1 | 125-170 g/L | 141 |
| | | Visit 3 | 125-170 g/L | 129 |
| 158001101 | 34/M | Screening Visit 1 | 125-170 g/L | 84(L) |
| | | Visit 3 | 125-170 g/L | 83(L) |
| | | Visit 4 | 125-170 g/L | 79(L) |
| | | Visit 5 | 125-170 g/L | 77(L) |
| | | Visit 6 | 125-170 g/L | 75(L) |
| | | Visit 8 | 125-170 g/L | 76(L) |
| 158001101 | 34/M | Visit 9 | 125-170 g/L | 82(L) |
| | | Visit 10 | 125-170 g/L | 84(L) |
| | | Visit 11 | 125-170 g/L | 81(L) |
| | | Visit 12 | 125-170 g/L | 84(L) |
| | | Visit 13 | 125-170 g/L | 85(L) |
| | | Tv1r | 125-170 g/L | 82(L) |
| | | Unscheduled Visit | 125-170 g/L | 82(L) |
| | | 215.01 | | |
| 158003101 | 66/M | Screening Visit 1 | 125-170 g/L | 144 |
| | | Visit 3 | 125-170 g/L | 137 |
| | | Visit 4 | 125-170 g/L | 142 |
| | | Visit 5 | 125-170 g/L | 139 |
| | | Visit 6 | 125-170 g/L | 138 |
| | | Visit 8 | 125-170 g/L | 135 |
| | | Visit 9 | 125-170 g/L | 137 |
| | | Visit 10 | 125-170 g/L | 138 |
| 158007101 | 20/M | Screening Visit 1 | 125-170 g/L | 72(L) |
| | | Visit 3 | 125-170 g/L | 75(L) |
| | | Visit 4 | 125-170 g/L | 89(L) |
| | | Visit 5 | 125-170 g/L | 98(L) |
| | | Visit 6 | 125-170 g/L | 110(L) |
| | | Visit 8 | 125-170 g/L | 119(L) |
| | | Visit 9 | 125-170 g/L | 114(L) |
| | | Visit 10 | 125-170 g/L | 120(L) |
| | | Visit 11 | 125-170 g/L | 115(L) |
| | | Visit 12 | 125-170 g/L | 117(L) |
| | | Visit 13 | 125-170 g/L | 110(L) |
| 158007101 | 20/M | Unscheduled Visit | | |
| | | 113.01 | | |
| 392004101 | 75/M | Screening Visit 1 | 125-170 g/L | 100(L) |
| | | Visit 3 | 125-170 g/L | 97(L) |
| | | Visit 4 | 125-170 g/L | 94(L) |
| | | Visit 5 | 125-170 g/L | 98(L) |
| 410001102 | 62/M | Screening Visit 1 | | |
| | | Unscheduled Visit | | |
| | | 102.01 | | |
| | | Unscheduled Visit | | |
| | | 102.02 | | |
| | | Visit 3 | 125-170 g/L | 66(L) |
| | | Visit 4 | 125-170 g/L | 78(L) |
| | | Visit 5 | 125-170 g/L | 78(L) |
| | | Visit 6 | 125-170 g/L | 76(L) |
| | | Visit 8 | 125-170 g/L | 79(L) |
| | | Visit 9 | 125-170 g/L | 66(L) |
| | | Visit 10 | 125-170 g/L | 89(L) |
| | | Visit 11 | 125-170 g/L | 76(L) |
| | | Visit 12 | 125-170 g/L | 76(L) |
| | | Visit 13 | 125-170 g/L | 75(L) |
| 410001104 | 33/M | Screening Visit 1 | 125-170 g/L | 81(L) |
| | | | 125-170 g/L | 62(L) |
| | | | 125-170 g/L | 75(L) |
| | | Screening Visit 1 | | |
| | | Unscheduled Visit | 125-170 g/L | 77(L) |
| | | 102.01 | | |
| | | Visit 3 | 125-170 g/L | 62(L) |
| 410001104 | 33/M | Visit 4 | 125-170 g/L | 71(L) |
| | | Visit 5 | 125-170 g/L | 55(L) |
| | | Visit 6 | 125-170 g/L | 54(L) |
| | | Visit 8 | 125-170 g/L | 53(L) |
| | | Visit 9 | 125-170 g/L | 55(L) |
| | | Visit 10 | 125-170 g/L | 68(L) |
| | | Visit 11 | 125-170 g/L | 53(L) |
| | | Visit 12 | 125-170 g/L | 56(L) |
| | | Visit 13 | 125-170 g/L | 69(L) |
| 410003101 | 47/M | Screening Visit 1 | 125-170 g/L | 113(L) |
| | | Visit 3 | 125-170 g/L | 120(L) |
| | | Visit 4 | 125-170 g/L | 127 |
| | | Visit 5 | 125-170 g/L | 137 |
| | | Visit 7 | 125-170 g/L | 123(L) |
| | | Visit 8 | | |
| | | Visit 9 | 125-170 g/L | 129 |
| | | Visit 10 | 125-170 g/L | 119(L) |
| | | Visit 11 | 125-170 g/L | 123(L) |
| | | Visit 12 | 125-170 g/L | 132 |
| | | Visit 13 | 125-170 g/L | 125 |
| | | Tv1r | 125-170 g/L | 121(L) |
| | | Tv2r | 125-170 g/L | 126 |
| 410004101 | 67/M | Screening Visit 1 | 125-170 g/L | 98(L) |
| | | Visit 3 | 125-170 g/L | 103(L) |
| | | Visit 4 | 125-170 g/L | 104(L) |
| | | Visit 5 | 125-170 g/L | 110(L) |
| 410004101 | 67/M | Visit 6 | 125-170 g/L | 109(L) |
| | | Visit 8 | 125-170 g/L | 110(L) |
| | | Visit 9 | 125-170 g/L | 115(L) |
| | | Visit 10 | 125-170 g/L | 118(L) |
| | | Visit 11 | 125-170 g/L | 116(L) |
| | | Visit 12 | 125-170 g/L | 122(L) |
| | | Visit 13 | 125-170 g/L | 121(L) |
| 410005101 | 20/F | Screening Visit 1 | 110-155 g/L | 88(L) |
| | | Visit 3 | 110-155 g/L | 81(L) |
| | | Visit 4 | 110-155 g/L | 75(L) |
| | | Visit 5 | 110-155 g/L | 92(L) |
| | | Visit 6 | 110-155 g/L | 90(L) |
| | | Visit 8 | 110-155 g/L | 89(L) |
| | | Visit 9 | 110-155 g/L | 98(L) |
| | | Visit 10 | 110-155 g/L | 96(L) |
| | | Visit 11 | 110-155 g/L | 95(L) |
| | | Visit 12 | 110-155 g/L | 100(L) |
| | | Visit 13 | 110-155 g/L | 99(L) |
| | | Unscheduled Visit | 110-155 g/L | 92(L) |
| | | 214.01 | | |
| | | Unscheduled Visit | 110-155 g/L | 86(L) |
| | | 215.01 | | |
| 410005103 | 23/F | Screening Visit 1 | 110-155 g/L | 75(L) |
| | | Visit 3 | 110-155 g/L | 90(L) |
| | | Visit 4 | 110-155 g/L | 89(L) |
| | | Visit 5 | 110-155 g/L | 101(L) |
| | | Visit 6 | 110-155 g/L | 96(L) |
| | | Visit 8 | 110-155 g/L | 104(L) |
| 410005103 | 23/F | Visit 9 | 110-155 g/L | 102(L) |
| | | Visit 10 | 110-155 g/L | 113 |
| | | Visit 11 | 110-155 g/L | 98(L) |
| 410008102 | 25/F | Screening Visit 1 | 110-155 g/L | 97(L) |
| | | Visit 3 | 110-155 g/L | 91(L) |
| | | Visit 4 | 110-155 g/L | 92(L) |
| | | Visit 5 | 110-155 g/L | 100(L) |
| | | Visit 6 | 110-155 g/L | 109(L) |
| | | Visit 8 | 110-155 g/L | 115 |
| | | Visit 9 | 110-155 g/L | 109(L) |
| | | Visit 10 | 110-155 g/L | 101(L) |
| | | Visit 11 | 110-155 g/L | 112 |
| | | Visit 12 | 110-155 g/L | 112 |
| | | Visit 13 | 110-155 g/L | 111 |
| | | Tv1r | 110-155 g/L | 112 |
| | | Tv2r | | |
| 410008103 | 51/F | Screening Visit 1 | 110-155 g/L | 93(L) |
| | | Visit 3 | | |
| | | Unscheduled Visit | 110-155 g/L | 96(L) |
| | | 103.02 | | |
| | | Visit 4 | 110-155 g/L | 95(L) |
| | | Visit 5 | 110-155 g/L | 104(L) |
| | | Visit 6 | 110-155 g/L | 110 |
| | | Visit 8 | 110-155 g/L | 97(L) |
| | | Visit 9 | 110-155 g/L | 118 |
| | | Visit 10 | 110-155 g/L | 103(L) |
| | | Visit 11 | 110-155 g/L | 100(L) |
| 410008103 | 51/F | Visit 12 | 110-155 g/L | 103(L) |
| | | Visit 13 | 110-155 g/L | 108(L) |
| 458001102 | 30/F | Screening Visit 1 | 110-155 g/L | 84(L) |
| | | Visit 3 | 110-155 g/L | 83(L) |
| | | Visit 4 | 110-155 g/L | 86(L) |
| | | Visit 5 | 110-155 g/L | 85(L) |
| | | Visit 6 | 110-155 g/L | 92(L) |
| | | Visit 8 | 110-155 g/L | 91(L) |
| | | Visit 9 | 110-155 g/L | 81(L) |
| | | Visit 10 | 110-155 g/L | 78(L) |
| | | Visit 11 | 110-155 g/L | 92(L) |
| | | Visit 12 | 110-155 g/L | 83(L) |
| | | Visit 13 | 110-155 g/L | 75(L) |
| | | Unscheduled Visit | 110-155 g/L | 82(L) |
| | | 113.02 | | |
| 458002102 | 45/F | Screening Visit 1 | 110-155 g/L | 79(L) |
| | | Unscheduled Visit | 110-155 g/L | 70(L) |
| | | 102.01 | | |
| | | Visit 3 | 110-155 g/L | 60(L) |
| | | Visit 4 | | |
| | | Unscheduled Visit | 110-155 g/L | 76(L) |
| | | 104.01 | | |
| | | Visit 5 | 110-155 g/L | 76(L) |
| | | Visit 6 | 110-155 g/L | 72(L) |
| | | Visit 8 | 110-155 g/L | 73(L) |
| | | Visit 9 | 110-155 g/L | 78(L) |
| | | Visit 10 | 110-155 g/L | 71(L) |
| | | Visit 11 | 110-155 g/L | 80(L) |
| | | Visit 12 | 110-155 g/L | 80(L) |
| 458002102 | 45/F | Visit 13 | 110-155 g/L | 80(L) |
| | | Unscheduled Visit | 110-155 g/L | 76(L) |
| | | 214.01 | | |
| 702001101 | 61/F | Screening Visit 1 | 110-155 g/L | 69(L) |
| | | Unscheduled Visit | 110-155 g/L | 84(L) |
| | | 101.02 | | |
| | | Visit 3 | 110-155 g/L | 49(L) |
| | | Visit 4 | 110-155 g/L | 54(L) |
| | | Visit 5 | 110-155 g/L | 72(L) |
| | | Visit 6 | 110-155 g/L | 58(L) |
| | | Visit 8 | 110-155 g/L | 67(L) |
| | | Visit 9 | 110-155 g/L | 71(L) |
| | | Visit 10 | 110-155 g/L | 62(L) |
| | | Visit 11 | 110-155 g/L | 81(L) |
| | | Visit 12 | 110-155 g/L | 64(L) |
| 724001102 | 20/F | Screening Visit 1 | 110-155 g/L | 120 |
| | | Visit 3 | 110-155 g/L | 103(L) |
| | | Visit 4 | 110-155 g/L | 102(L) |
| | | Visit 5 | 110-155 g/L | 96(L) |
| | | Visit 6 | 110-155 g/L | 96(L) |
| | | Visit 8 | 110-155 g/L | 97(L) |
| 764002101 | 36/F | Screening Visit 1 | 110-155 g/L | 74(L) |
| | | Unscheduled Visit | 110-155 g/L | 94(L) |
| | | 102.01 | | |
| | | Visit 3 | 110-155 g/L | 62(L) |
| | | Visit 4 | 110-155 g/L | 59(L) |
| | | Visit 5 | 110-155 g/L | 82(L) |
| | | Visit 6 | 110-155 g/L | 63(L) |
| 764002101 | 36/F | Visit 8 | 110-155 g/L | 81(L) |
| 764002103 | 32/M | Screening Visit 1 | 125-170 g/L | 108(L) |
| | | Visit 3 | 125-170 g/L | 73(L) |
| | | Visit 4 | 125-170 g/L | 74(L) |
| | | Visit 5 | 125-170 g/L | 73(L) |
| | | Visit 6 | 125-170 g/L | 90(L) |
| 764004102 | 42/F | Screening Visit 1 | 110-155 g/L | 93(L) |
| | | Visit 3 | 110-155 g/L | 82(L) |
| | | Visit 4 | 110-155 g/L | 75(L) |

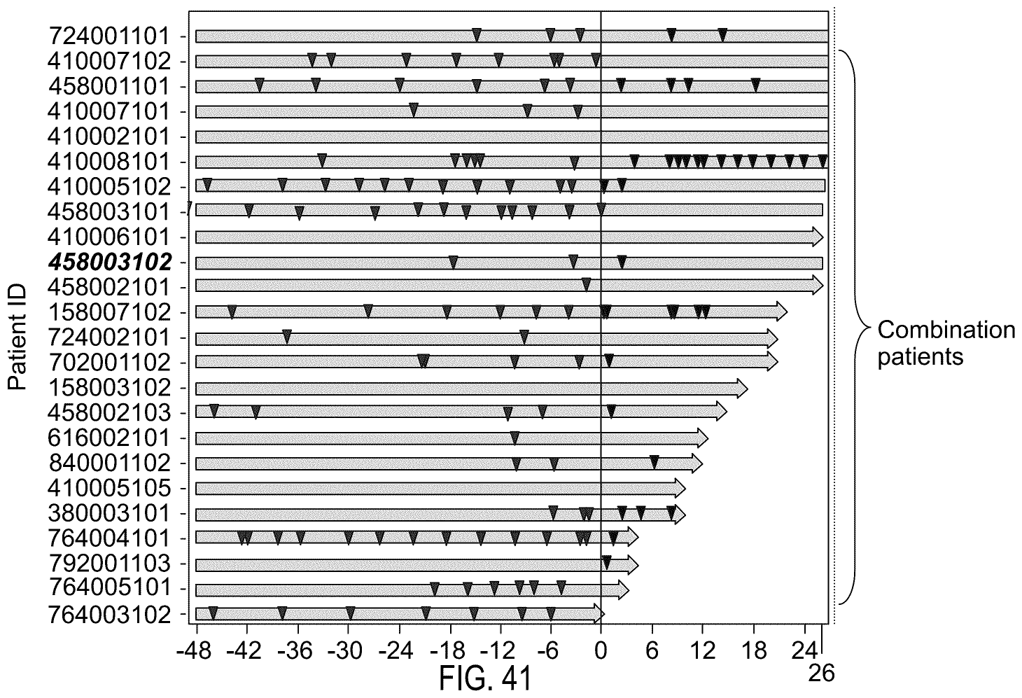

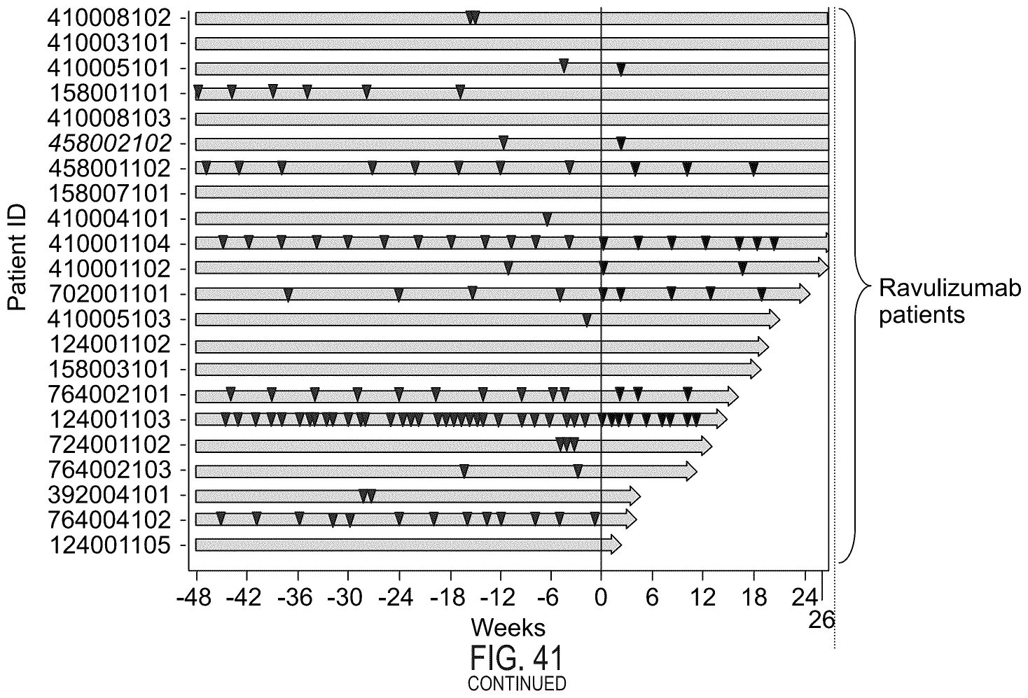

Transfusion avoidance was similar among week 26 completers. See Table 7-14. Transfusions can be driven by hemolysis (intravascular hemolysis, extravascular hemolytsis), bone marrow insufficiency and other factors such as blood loss. There may have been an imbalance in other factors given small sample size. See also, the summary of per protocol transfusions between groups in FIG. 41.

TABLE 7-14
Summary of Transfusion Received by Patients

| | | On study pts with |
|---|---|---|
| | On study pts with transfusion, all | transfusion |
| | (whether or not criteria met) | (completers) |
| Combo | 46% | 36% |
| Ravulizumab | 32% | 36% |

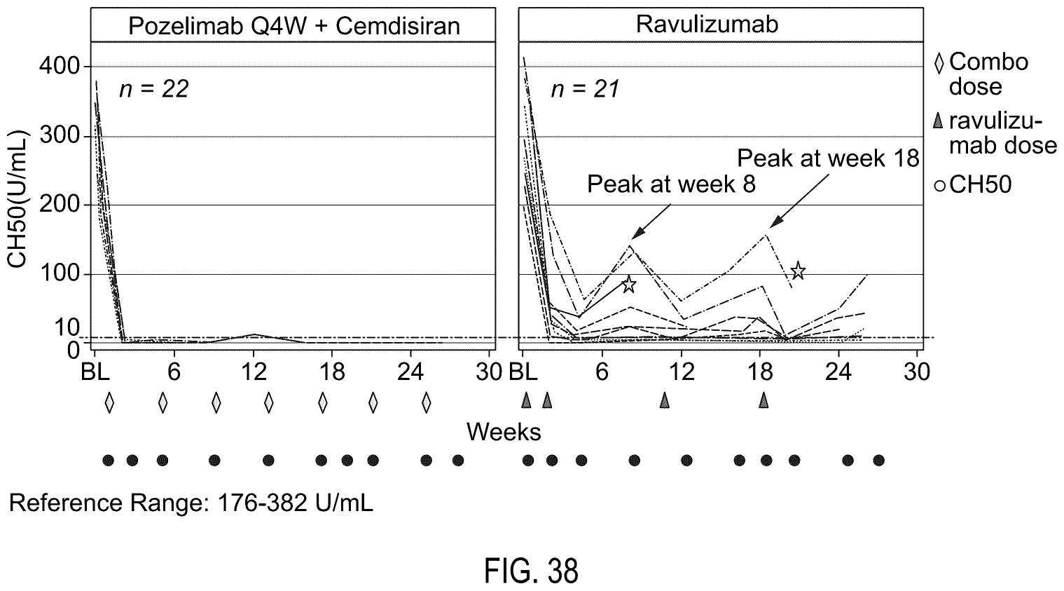

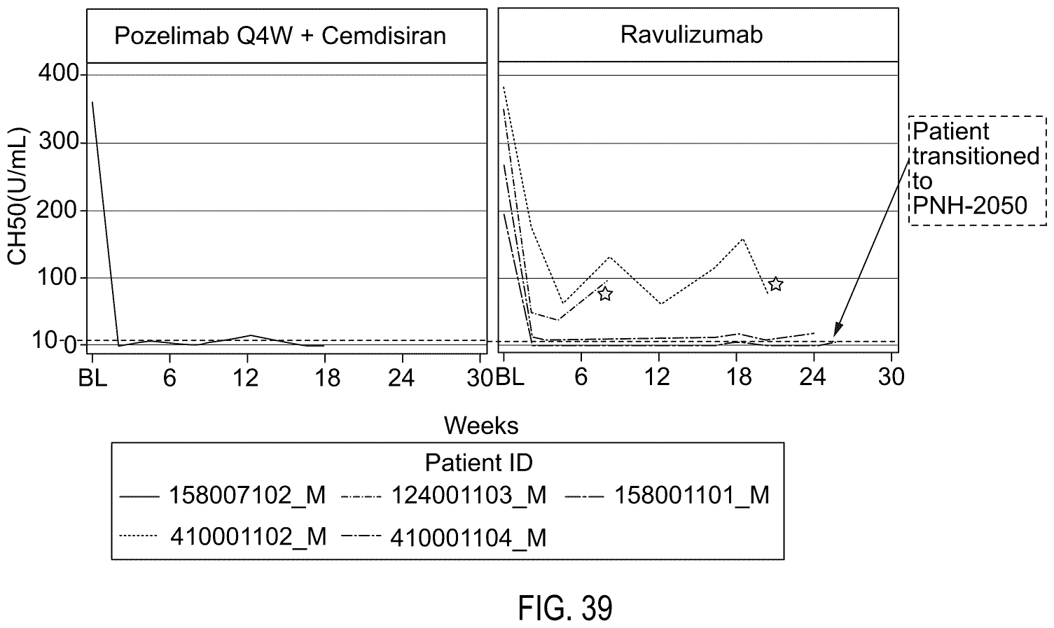

Complement hemolytic assay (CH50) is an ex vivo functional assay measuring the activity of terminal complement in patient's blood and measures the ability of an individual's blood to lyse sensitized (antibody coated) sheep red blood cells (RBCs). Result are reported as the reciprocal of the dilution of serum required to lyse 50% of antibody coated sheep RBCs. If elevated, terminal complement activity is present. The assay is sensitive to reduction or absence of any component of the classical pathway (C1, C2, C3, C4) or terminal complement activity (C5 through 9). The CH50 assay results of individual patients per visit are set forth in Table 7-15. Out of range result flags (H=high and L=low) are included. CH50 data for Ravulizumab patients revealed a saw-tooth pattern that aligned with drug trough levels, while CH50 for Combo patients remained close to 100% inhibition at all timepoints. See FIG. 38. CH50 data from inadequate responders on Ravulizumab showed incomplete terminal complement inhibition. See FIG. 39. The patient who transitioned to the combo treatment is indicated (Subject ID 158-001-101).

TABLE 7-15
Clinical Laboratory Test-CH50 (Full Analysis Set)

| | | | | |
|---|---|---|---|---|
| | | | Reference | Result in Std |
| | | | Range in Std | Units (Out of |
| Subject ID | Age/Sex | Visit | Units | Range Flag) |

Pozelimab q4w + Cemdisiran

| | | | | |
|---|---|---|---|---|
| 158003102 | 39/F | Visit 3 | 176-382 | 340 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| 158007102 | 66/M | Visit 3 | 176-382 | 359 |
| | | | U/mL | |
| | | Visit 4 | 176-382 | 0(L) |
| | | | U/mL | |
| | | Visit 5 | 176-382 | 6(L) |
| | | | U/mL | |
| | | Visit 6 | 176-382 | 1(L) |
| | | | U/mL | |
| | | Visit 8 | 176-382 | 14(L) |
| | | | U/mL | |
| | | Visit 9 | 176-382 | 1(L) |
| | | | U/mL | |
| | | Visit 10 | 176-382 | 0(L) |
| | | | U/mL | |
| 380003101 | 40/F | Visit 3 | 176-382 | 304 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | | |
| 410002101 | 65/F | Visit 3 | 176-382 | 342 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 410005102 | 61/M | Visit 3 | 176-382 | 199 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 410005105 | 39/M | Visit 3 | 176-382 | 250 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | | |
| 410006101 | 36/F | Visit 3 | 176-382 | 306 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 410007101 | 40/M | Visit 3 | 176-382 | 248 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 410007102 | 58/M | Visit 3 | 176-382 | 304 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 410008101 | 73/M | Visit 3 | 176-382 | 290 |
| | | | U/mL | |
| | | Visit 4 | 176-382 | 0(L) |
| | | | U/mL | |
| | | Visit 5 | 176-382 | 0(L) |
| | | | U/mL | |
| | | Visit 7 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Unscheduled | 176-382 | 0(L) |
| | | Visit 111.01 | U/mL | |
| | | Visit 12 | 176-382 | 0(L) |
| | | | U/mL | |
| | | Visit 13 | 176-382 | 0(L) |
| | | | U/mL | |
| 458001101 | 44/M | Visit 3 | 176-382 | 242 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 458002101 | 46/M | Visit 3 | 176-382 | 350 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 458002103 | 43/F | Visit 3 | 176-382 | 278 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | | |
| 458003101 | 39/M | Unscheduled | 176-382 | 304 |
| | | Visit 101.01 | U/mL | |
| | | Visit 3 | 176-382 | 236 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 458003102 | 29/M | Visit 3 | 176-382 | 208 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 2(L) |
| | | | U/mL | |
| | | Visit 11 | 176-382 | 0(L) |
| | | | U/mL | |
| | | Visit 12 | | |
| | | Visit 13 | | |
| 616002101 | 21/F | Visit 3 | 176-382 | 311 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | | |
| 702001102 | 47/M | Visit 3 | 176-382 | 267 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | | |
| | | Visit 11 | | |
| 724001101 | 48/F | Visit 3 | 176-382 | 256 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| 724002101 | 87/F | Visit 3 | 176-382 | 314 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 11 | | |
| 764004101 | 56/M | Visit 3 | 176-382 | 258 |
| | | | U/mL | |
| | | Visit 4 | | |
| 764005101 | 50/F | Visit 3 | | |
| | | Visit 4 | | |
| 792001103 | 21/F | Visit 3 | 176-382 | 381 |
| | | | U/mL | |
| | | Visit 4 | 176-382 | 0(L) |
| | | | U/mL | |
| | | Visit 5 | | |
| 840001102 | 23/F | Visit 3 | 176-382 | 229 |
| | | | U/mL | |
| | | Visit 6 | | |

Ravulizumab

| | | | | |
|---|---|---|---|---|
| 124001102 | 58/M | Visit 3 | 176-382 | 274 |
| | | | U/mL | |
| | | Visit 4 | 176-382 | 0(L) |
| | | | U/mL | |
| | | Visit 5 | 176-382 | 0(L) |
| | | | U/mL | |
| | | Visit 7 | 176-382 | 0(L) |
| | | | U/mL | |
| | | Visit 9 | | |
| 124001103 | 21/M | Visit 3 | 176-382 | 350 |
| | | | U/mL | |
| | | Visit 4 | 176-382 | 48(L) |
| | | | U/mL | |
| | | Visit 5 | 176-382 | 37(L) |
| | | | U/mL | |
| | | Visit 7 | 176-382 | 96(L) |
| | | | U/mL | |
| | | Visit 9 | | |
| 124001105 | 36/M | Visit 3 | | |
| 158001101 | 34/M | Visit 3 | 176-382 | 196 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 1(L) |
| | | | U/mL | |
| | | Visit 10 | 176-382 | 6(L) |
| | | | U/mL | |
| | | Visit 11 | 176-382 | 0(L) |
| | | | U/mL | |
| | | Visit 12 | 176-382 | 1(L) |
| | | | U/mL | |
| | | Visit 13 | 176-382 | 6(L) |
| | | | U/mL | |
| | | Tv1r | 176-382 | 1(L) |
| | | | U/mL | |
| | | Unscheduled | | |
| | | Visit 215.01 | | |
| 158003101 | 66/M | Visit 3 | 176-382 | 228 |
| | | | U/mL | |
| | | Visit 4 | 176-382 | 13(L) |
| | | | U/mL | |
| | | Visit 5 | 176-382 | 0(L) |
| | | | U/mL | |
| | | Visit 6 | 176-382 | 2(L) |
| | | | U/mL | |
| | | Visit 8 | 176-382 | 1(L) |
| | | | U/mL | |
| | | Visit 9 | | |
| | | Visit 10 | | |
| 158007101 | 20/M | Visit 3 | 176-382 | 257 |
| | | | U/mL | |
| | | Visit 4 | 176-382 | 11(L) |
| | | | U/mL | |
| | | Visit 5 | 176-382 | 1(L) |
| | | | U/mL | |
| | | Visit 6 | 176-382 | 5(L) |
| | | | U/mL | |
| | | Visit 8 | 176-382 | 0(L) |
| | | | U/mL | |
| | | Visit 9 | 176-382 | 1(L) |
| | | | U/mL | |
| | | Visit 10 | 176-382 | 6(L) |
| | | | U/mL | |
| | | Visit 11 | 176-382 | 0(L) |
| | | | U/mL | |
| | | Visit 12 | 176-382 | 4(L) |
| | | | U/mL | |
| | | Visit 13 | 176-382 | 4(L) |
| | | | U/mL | |
| | | Unscheduled | | |
| | | Visit 113.01 | | |
| 392004101 | 75/M | Visit 3 | 176-382 | 303 |
| | | | U/mL | |
| | | Visit 4 | | |
| | | Visit 5 | | |
| 410001102 | 62/M | Visit 3 | 176-382 | 384(H) |
| | | | U/mL | |
| | | Visit 4 | 176-382 | 175(L) |
| | | | U/mL | |
| | | Visit 5 | 176-382 | 62(L) |
| | | | U/mL | |
| | | Visit 6 | 176-382 | 130(L) |
| | | | U/mL | |
| | | Visit 8 | 176-382 | 61(L) |
| | | | U/mL | |
| | | Visit 9 | 176-382 | 117(L) |
| | | | U/mL | |
| | | Visit 10 | 176-382 | 158(L) |
| | | | U/mL | |
| | | Visit 11 | 176-382 | 76(L) |
| | | | U/mL | |
| | | Visit 12 | | |
| | | Visit 13 | | |
| 410001104 | 33/M | Visit 3 | 176-382 | 268 |
| | | | U/mL | |
| | | Visit 4 | 176-382 | 13(L) |
| | | | U/mL | |
| | | Visit 5 | 176-382 | 6(L) |
| | | | U/mL | |
| | | Visit 6 | 176-382 | 9(L) |
| | | | U/mL | |
| | | Visit 8 | 176-382 | 9(L) |
| | | | U/mL | |
| | | Visit 9 | 176-382 | 11(L) |
| | | | U/mL | |
| | | Visit 10 | 176-382 | 17(L) |
| | | | U/mL | |
| | | Visit 11 | 176-382 | 7(L) |
| | | | U/mL | |
| | | Visit 12 | 176-382 | 18(L) |
| | | | U/mL | |
| | | Visit 13 | | |
| 410003101 | 47/M | Visit 3 | 176-382 | 254 |
| | | | U/mL | |
| | | Visit 4 | 176-382 | 10(L) |
| | | | U/mL | |
| | | Visit 5 | 176-382 | 0(L) |
| | | | U/mL | |
| | | Visit 7 | 176-382 | 0(L) |
| | | | U/mL | |
| | | Visit 8 | 176-382 | 0(L) |
| | | | U/mL | |
| | | Visit 9 | 176-382 | 2(L) |
| | | | U/mL | |
| | | Visit 10 | 176-382 | 8(L) |
| | | | U/mL | |
| | | Visit 11 | 176-382 | 0(L) |
| | | | U/mL | |
| | | Visit 12 | 176-382 | 3(L) |
| | | | U/mL | |
| | | Visit 13 | 176-382 | 2(L) |
| | | | U/mL | |
| | | Tv1r | 176-382 | 1(L) |
| | | | U/mL | |
| | | Tv2r | 176-382 | 0(L) |
| | | | U/mL | |
| 410004101 | 67/M | Visit 3 | 176-382 | 290 |
| | | | U/mL | |
| | | Visit 4 | 176-382 | 8(L) |
| | | | U/mL | |
| | | Visit 5 | 176-382 | 2(L) |
| | | | U/mL | |
| | | Visit 6 | 176-382 | 5(L) |
| | | | U/mL | |
| | | Visit 8 | 176-382 | 2(L) |
| | | | U/mL | |
| | | Visit 9 | 176-382 | 4(L) |
| | | | U/mL | |
| | | Visit 10 | 176-382 | 8(L) |
| | | | U/mL | |
| | | Visit 11 | 176-382 | 3(L) |
| | | | U/mL | |
| | | Visit 12 | 176-382 | 9(L) |
| | | | U/mL | |
| | | Visit 13 | 176-382 | 12(L) |
| | | | U/mL | |
| 410005101 | 20/F | Visit 3 | 176-382 | 275 |
| | | | U/mL | |
| | | Visit 4 | 176-382 | 0(L) |
| | | | U/mL | |
| | | Visit 5 | 176-382 | 0(L) |
| | | | U/mL | |
| | | Visit 6 | 176-382 | 0(L) |
| | | | U/mL | |
| | | Visit 8 | 176-382 | 0(L) |
| | | | U/mL | |
| | | Visit 9 | 176-382 | 0(L) |
| | | | U/mL | |
| | | Visit 10 | 176-382 | 0(L) |
| | | | U/mL | |
| | | Visit 11 | 176-382 | 0(L) |
| | | | U/mL | |
| | | Visit 12 | 176-382 | 0(L) |
| | | | U/mL | |
| | | Visit 13 | 176-382 | 0(L) |
| | | | U/mL | |
| | | Unscheduled | 176-382 | 0(L) |
| | | Visit 214.01 | U/mL | |
| | | Unscheduled | 176-382 | 0(L) |
| | | Visit 215.01 | U/mL | |
| 410005103 | 23/F | Visit 3 | 176-382 | 248 |
| | | | U/mL | |
| | | Visit 4 | 176-382 | 39(L) |
| | | | U/mL | |
| | | Visit 5 | 176-382 | 4(L) |
| | | | U/mL | |
| | | Visit 6 | 176-382 | 8(L) |
| | | | U/mL | |
| | | Visit 8 | 176-382 | 1(L) |
| | | | U/mL | |
| | | Visit 9 | 176-382 | 10(L) |
| | | | U/mL | |
| | | Visit 10 | | |
| | | Visit 11 | | |
| 410008102 | 25/F | Visit 3 | 176-382 | 276 |
| | | | U/mL | |
| | | Visit 4 | 176-382 | 8(L) |
| | | | U/mL | |
| | | Visit 5 | 176-382 | 0(L) |
| | | | U/mL | |
| | | Visit 6 | 176-382 | 1(L) |
| | | | U/mL | |
| | | Visit 8 | 176-382 | 1(L) |
| | | | U/mL | |
| | | Visit 9 | 176-382 | 2(L) |
| | | | U/mL | |
| | | Visit 10 | 176-382 | 8(L) |
| | | | U/mL | |
| | | Visit 11 | 176-382 | 0(L) |
| | | | U/mL | |
| | | Visit 12 | 176-382 | 3(L) |
| | | | U/mL | |
| | | Visit 13 | 176-382 | 21(L) |
| | | | U/mL | |
| | | Tv1r | 176-382 | 0(L) |
| | | | U/mL | |
| | | Tv2r | | |
| 410008103 | 51/F | Visit 3 | 176-382 | 276 |
| | | | U/mL | |
| | | Unscheduled | 176-382 | 36(L) |
| | | Visit 103.02 | U/mL | |
| | | Visit 4 | 176-382 | 23(L) |
| | | | U/mL | |
| | | Visit 5 | 176-382 | 1(L) |
| | | | U/mL | |
| | | Visit 6 | 176-382 | 19(L) |
| | | | U/mL | |
| | | Visit 8 | 176-382 | 2(L) |
| | | | U/mL | |
| | | Visit 9 | 176-382 | 9(L) |
| | | | U/mL | |
| | | Visit 10 | 176-382 | 17(L) |
| | | | U/mL | |
| | | Visit 11 | 176-382 | 1(L) |
| | | | U/mL | |
| | | Visit 12 | 176-382 | 10(L) |
| | | | U/mL | |
| | | Visit 13 | 176-382 | 15(L) |
| | | | U/mL | |
| 458001102 | 30/F | Visit 3 | 176-382 | 291 |
| | | | U/mL | |
| | | Visit 4 | 176-382 | 19(L) |
| | | | U/mL | |
| | | Visit 5 | 176-382 | 1(L) |
| | | | U/mL | |
| | | Visit 6 | 176-382 | 24(L) |
| | | | U/mL | |
| | | Visit 8 | 176-382 | 22(L) |
| | | | U/mL | |
| | | Visit 9 | 176-382 | 18(L) |
| | | | U/mL | |
| | | Visit 10 | 176-382 | 39(L) |
| | | | U/mL | |
| | | Visit 11 | 176-382 | 3(L) |
| | | | U/mL | |
| | | Visit 12 | 176-382 | 37(L) |
| | | | U/mL | |
| | | Visit 13 | 176-382 | 45(L) |
| | | | U/mL | |
| | | Unscheduled | | |
| | | Visit 113.02 | | |
| 458002102 | 45/F | Visit 3 | 176-382 | 419(H) |
| | | | U/mL | |
| | | Visit 4 | 176-382 | 130(L) |
| | | | U/mL | |
| | | Visit 5 | 176-382 | 37(L) |
| | | | U/mL | |
| | | Visit 6 | 176-382 | 142(L) |
| | | | U/mL | |
| | | Visit 8 | 176-382 | 33(L) |
| | | | U/mL | |
| | | Visit 9 | 176-382 | 66(L) |
| | | | U/mL | |
| | | Visit 10 | 176-382 | 83(L) |
| | | | U/mL | |
| | | Visit 11 | 176-382 | 12(L) |
| | | | U/mL | |
| | | Visit 12 | 176-382 | 49(L) |
| | | | U/mL | |
| | | Visit 13 | 176-382 | 99(L) |
| | | | U/mL | |
| | | Unscheduled | | |
| | | Visit 214.01 | | |
| 702001101 | 61/F | Visit 3 | 176-382 | 402(H) |
| | | | U/mL | |
| | | Visit 4 | 176-382 | 27(L) |
| | | | U/mL | |
| | | Visit 5 | 176-382 | 12(L) |
| | | | U/mL | |
| | | Visit 6 | 176-382 | 24(L) |
| | | | U/mL | |
| | | Visit 8 | 176-382 | 6(L) |
| | | | U/mL | |
| | | Visit 9 | 176-382 | 37(L) |
| | | | U/mL | |
| | | Visit 10 | 176-382 | 36(L) |
| | | | U/mL | |
| | | Visit 11 | 176-382 | 9(L) |
| | | | U/mL | |
| | | Visit 12 | | |
| 724001102 | 20/F | Visit 3 | 176-382 | 257 |
| | | | U/mL | |
| | | Visit 4 | 176-382 | 13(L) |
| | | | U/mL | |
| | | Visit 5 | 176-382 | 3(L) |
| | | | U/mL | |
| | | Visit 6 | | |
| | | Visit 8 | | |
| 764002101 | 36/F | Visit 3 | 176-382 | 343 |
| | | | U/mL | |
| | | Visit 4 | 176-382 | 58(L) |
| | | | U/mL | |
| | | Visit 5 | 176-382 | 19(L) |
| | | | U/mL | |
| | | Visit 6 | 176-382 | 51(L) |
| | | | U/mL | |
| | | Visit 8 | 176-382 | 26(L) |
| | | | U/mL | |
| 764002103 | 32/M | Visit 3 | 176-382 | 218 |
| | | | U/mL | |
| | | Visit 4 | 176-382 | 20(L) |
| | | | U/mL | |
| | | Visit 5 | 176-382 | 9(L) |
| | | | U/mL | |
| | | Visit 6 | | |
| 764004102 | 42/F | Visit 3 | 176-382 | 303 |
| | | | U/mL | |
| | | Visit 4 | | |

No new safety signals emerge based on treatment emergent adverse events comparing combo vs Ravulizumab:
% subjects with TEAEs similar in both arms, but higher #TEAEs w/ combo driven by a large #of TEAEs reported in one patient (n=21)
2 subjects with serious and severe TEAEs in combo arm (not related to study treatment)
   SAEs
      1 patient with fever and seizure (both at Day 6) with hemolytic crisis (Day 4)
      1 patient with lower extremity cellulitis (severe); combo sub optimal responder
   Additional severe TEAEs in the 1 patient with numerous TEAEs
      Non-serious TEAEs: worsening of anemia (3), worsening of neutropenia

### See Table 7-16.

TABLE 7-16
Overview of Treatment-Emergent Adverse Events-Occurred in
the OLTP (baseline to Week 26-Analysis Set for Cohort A)

| | Pozelimab Q4W + | |
|---|---|---|
| | Cemdisiran | Ravulizumab |
| Total Number of TEAEs | N = 24 | N = 22 |
| Total Number of serious | 62 | 34 |
| TEAEs | | |
| Total Number of related | 4 | 0 |
| TEAEs | | |
| Subjects with any TEAE | 10 | 4 |
| Subjects with any serious | 16 (66.7%) | 14 (63.6%) |
| TEAE | | |
| Subjects with any | 2 (8.3%) | 0 |
| severeTEAE | | |
| Subjects with any TEAE | 2 (8.3%) | 0 |
| leading to discontinuation | | |
| of any study drug | | |
| Subjects with any TEAE | 0 | 0 |
| leading to death | | |

### Example 8: Viscosity Reducers in Co-Formulations Containing Cemdisiran & REGN3918

This study was initiated to evaluate the effects of potential excipients on viscosity. Two base co-formulations, one containing 120 mg/mL Cemdisiran, 120 mg/mL Pozelimab, 15 mM histidine, pH 6.2 and another containing 75 mg/mL Cemdisiran, 150 mg/mL Pozelimab, 15 mM histidine, pH 6.2 were manufactured with a potential viscosity reducer. List of viscosity reducers and concentrations tested are shown below:
75 mM arginine hydrochloride
5 mM sodium adipate
75 mM sodium chloride
75 mM lysine hydrochloride
75 mM sodium aspartate
75 mM proline
35 mM histidine added for a total of 50 mM histidine in final co-formulations
50 mM caffeine
50 mM phenylalanine
75 mM triethyl citrate

Co-formulations without additional excipient were also manufactured and used as a control for this study.

### Results

Viscosity at 20° C. was accessed by an automatic viscometer for all co-formulations. Table 8-1 shows the reduction of viscosity for each co-formulation compared to control. Maximum viscosity reduction observed was approximately a third. All excipients tested were effective viscosity reducers for co-formulations that contained 120 mg/mL Cemdisiran and 120 mg/mL Pozelimab, while only a subset was effective for co-formulations that contained 75 mg/mL Cemdisiran and 150 mg/mL Pozelimab. A common property of ineffective viscosity reducer was cyclic structure vs a effective viscosity reducer which was the salt form for co-formulations that contained 75 mg/mL Cemdisiran and 150 mg/mL Pozelimab. Intermolecular interactions possibly dominated by electrostatics, given effectiveness of sodium chloride and salt forms of amino acids (either Na⁺ or HCl) for both co-formulations.

Purity of both Pozelimab and Cemdisiran was measured after ≥1 month storage at 2-8° C. and compared to source material used for manufacturing co-formulations. Size exclusion ultra-performance liquid chromatography using a fluorescence detector was performed to access Pozelimab stability. See Table 8-2. Denaturing anion exchange ultra-performance liquid chromatography was performed to access Cemdisiran stability. See Table 8-3. All formulations showed no appreciable change in purity.

TABLE 8-1
Viscosity of Cemdisiran & Pozelimab with Various Excipients (viscosity (at 20°
C.) was measured and results compared to control (without viscosity reducer))

| | 1:1 base formulation: | | 1:2 base formulation: | |
|---|---|---|---|---|
| | 120 mg/mL Cemdisiran, | | 75 mg/mL Cemdisiran, | |
| | 120 mg/mL Pozelimab, | | 150 mg/mL Pozelimab, | |
| | 15 mM histidine, pH 6.2 | | 15 mM histidine, pH 6.2 | |
| | | % Reduction | | % Reduction |
| | Viscosity at | compared | Viscosity at | compared |
| Viscosity Reducer | 20° C. (cP) | to control | 20° C. (cP) | to control |
| None (control) | 29.1 | NA | 29.8 | NA |
| 75 mM arginine | 19.7 | −32 | 20.8 | −30* |
| hydrochloride | | | | |
| 75 mM sodium adipate | 18.6 | −36 | 21.3 | −29* |
| 75 mM sodium chloride | 18.7 | −36 | 19.8 | −34* |
| 75 mM lysine | 19.6 | −33 | 20.9 | −30* |
| hydrochloride | | | | |
| 75 mM sodium aspartate | 19.9 | −32 | 21.7 | −27* |
| 75 mM proline | 22.7 | −22 | 30.0 | +1 |
| 50 mM histidine (total) | 24.5 | −16 | 30.4 | +2 |
| 50 mM caffeine | 24.7 | −15 | 32.0 | +7 |
| 50 mM phenylalanine | 24.7 | −15 | 30.7 | +3 |
| 75 mM triethyl citrate | 27.0 | −7 | 30.9 | +4 |

*Intermolecular interactions possibly dominated by electrostatics since salts are effective at reducing viscosity

TABLE 8-2
SEC-FLR-UPLC (Size exclusion ultra-performance liquid chromatography
using a fluorescence detector) Analysis to Access REGN3918 stability

| | 1:1 base formulation: | | 1:2 base formulation: | |
|---|---|---|---|---|
| | 120 mg/mL Cemdisiran, | | 75 mg/mL Cemdisiran, | |
| | 120 mg/mL Pozelimab, | | 150 mg/mL Pozelimab, | |
| | 15 mM histidine, pH 6.2 | | 15 mM histidine, pH 6.2 | |
| | REGN3918 | Delta from | REGN3918 | Delta from |
| Viscosity Reducer | Purity (%) | control | Purity (%) | control |
| 75 mM arginine hydrochloride | 96.9 | 0.6 | 96.7 | 0.4 |
| 75 mM sodium adipate | 96.5 | 0.3 | 96.5 | 0.3 |
| 75 mM sodium chloride | 96.6 | 0.4 | 96.7 | 0.5 |
| 75 mM lysine hydrochloride | 96.8 | 0.5 | 96.9 | 0.6 |
| 75 mM sodium aspartate | 96.7 | 0.4 | 96.6 | 0.3 |
| 75 mM proline | 96.6 | 0.3 | 96.8 | 0.5 |
| 50 mM histidine (total) | 96.4 | 0.2 | 96.7 | 0.4 |
| 50 mM caffeine | 96.6 | 0.4 | 96.7 | 0.4 |
| 50 mM phenylalanine | 96.2 | 0.0 | 96.3 | 0.1 |
| 75 mM triethyl citrate | 96.5 | 0.3 | 96.7 | 0.4 |
| Control (compared to source | 96.2 | NA | 96.2 | NA |
| material use for manufacturing | | | | |
| formulations) | | | | |

TABLE 8-3
dAEX-UPLC (denaturing anion exchange ultra-performance liquid
chromatography) Analysis to Access Cemdisiran Purity

| | 1:1 base formulation: | | 1:2 base formulation: | |
|---|---|---|---|---|
| | 120 mg/mL Cemdisiran, | | 75 mg/mL Cemdisiran, | |
| | 120 mg/mL Pozelimab, | | 150 mg/mL Pozelimab, | |
| | 15 mM histidine, pH 6.2 | | 15 mM histidine, pH 6.2 | |
| | Cemdisiran | Delta from | Cemdisiran | Delta from |
| Viscosity Reducer | Purity (%) | control | Purity (%) | control |
| 75 mM arginine | 94.4 | −1.0 | 94.7 | −0.7 |
| hydrochloride | | | | |
| 75 mM sodium adipate | 94.8 | −0.5 | 94.7 | −0.6 |
| 75 mM sodium chloride | 94.5 | −0.8 | 94.3 | −1.1 |
| 75 mM lysine hydrochloride | 94.5 | −0.9 | 94.1 | −1.3 |
| 75 mM sodium aspartate | 94.8 | −0.5 | 94.6 | −0.7 |
| 75 mM proline | 94.7 | −0.7 | 94.0 | −1.4 |
| 50 mM histidine (total) | 94.7 | −0.6 | 95.3 | −0.1 |
| 50 mM caffeine | 94.8 | −0.5 | 94.6 | −0.7 |
| 50 mM phenylalanine | 94.6 | −0.7 | 94.6 | −0.8 |
| 75 mM triethyl citrate | 94.7 | −0.6 | 94.6 | −0.8 |
| Control (compared to source | 95.4 | NA | 95.4 | NA |
| material use for | | | | |
| manufacturing formulations) | | | | |

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present disclosure is not to be limited in scope by the specific embodiments described herein. Indeed, various modifications of the disclosure in addition to those described herein will become apparent to those skilled in the art from the foregoing description and accompanying figures. Such modifications are intended to fall within the scope of the appended claims.

1. A co-formulation comprising:
a C5 iRNA which is conjugated to a ligand that comprises one or more terminal N-Acetylgalactosamine (GalNAc) and/or N-acetylglucosamine (GlcNAc) residues;
an antibody or antigen-binding fragment thereof that binds specifically to C5 (anti-C5) which is isolated from a mammalian host cell; and
a pharmaceutically acceptable carrier;
wherein the co-formulation has a pH of greater than or less than about 6.

2. The co-formulation of claim 1, comprising:
a C5 iRNA;
an antibody or antigen-binding fragment thereof that binds specifically to C5;
a buffer;
a viscosity reducer;
a stabilizer; and
a non-ionic surfactant, and
pH of greater than or less than about 6.

3. The co-formulation of claim 1, which has a pH of about 6.5 or a pH within not less than 0.5 of 6.0.

4. The co-formulation of claim 1, comprising a buffer which is a histidine-based buffer, a citrate-based buffer, a phosphate-based buffer and/or an acetate-based buffer.

5. The co-formulation of claim 1, which comprises about 10-35, 35-45, 20-50, 20, 25, 30, 35, 40, 45 or 50 mM buffer.

6. The co-formulation of claim 1, comprising a viscosity reducer which is an inorganic salt and/or an amino acid.

7. (canceled)

8. The co-formulation of claim 1, which comprises about 20-140, 20, 25, 30, 35, 40, 45, 50, 55, 60, 65, 70, 75, 80, 85, 90, 95, 100, 105, 110, 115, 120, 125, 130, 135 or 140 mM viscosity reducer.

9. The co-formulation of claim 1, comprising a stabilizer which is a polyol or sugar.

10. The co-formulation of claim 1, comprising a stabilizer which is trehalose, sorbitol, mannitol, taurine, propane sulfonic acid, L-proline, sucrose, glycerol, threitol, maltitol, polyethylene glycol (PEG), and/or PEG3350.

11. The co-formulation of claim 1, which comprises about 0.8-3.6, 0.8, 0.9, 1.0, 1.25, 1.50, 2.0, 2.25, 2.50, 2.75, 3.00, 3.1, 3.2, 3.3, 3.4, 3.5 or 3.6% (w/v) stabilizer.

12. (canceled)

13. (canceled)

14. The co-formulation of claim 1, which comprises about 0.025, 0.05, 0.075, 0.1, 0.125, 0.15, 0.175% (w/v) non-ionic surfactant.

15. The co-formulation of claim 1, further comprising one or more viscosity reducers.

16. The co-formulation of claim 1, further comprising one or more viscosity reducers at a concentration of about 5 mM to about 100 mM each.

17. (canceled)

18. The co-formulation of claim 15, wherein the viscosity reducer is a one or more of: an amino acid, a dicarboxylic acid, an inorganic salt, an ester of citric acid and/or a xanthine.

19. The co-formulation of claim 15, wherein the viscosity reducer is one or more of:
arginine;
adipic acid;
NaCl;
lysine;
proline;
histidine;
caffeine;
phenylalanine; and
triethyl citrate.

20-22. (canceled)

23. The co-formulation of claim 10, which comprises about 94% or more C5 iRNA purity as assessed by anion exchange chromatography after about 1 month at 2-8° C.

24. The co-formulation of claim 1, which comprises a 1:1 ratio of milligrams per milliliter concentration of C5 iRNA and anti-C5 antibody or antigen-binding fragment.

25-29. (canceled)

30. The co-formulation of claim 1, wherein the antibody or antigen-binding fragment thereof that binds specifically to C5 (anti-C5) comprises:
(1) a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10;
(2) a heavy chain variable region (HCVR) that comprises the HCDR1, HCDR2 and HCDR3 of a HCVR that comprises the amino acid sequence set forth in SEQ ID NO: 18, and a light chain variable region (LCVR) that comprises the LCDR1, LCDR2 and LCDR3 of a LCVR that comprises the amino acid sequence set forth in SEQ ID NO: 26;
(3) a heavy chain variable region (HCVR) that comprises the HCDR1, HCDR2 and HCDR3 of a HCVR that comprises the amino acid sequence set forth in SEQ ID NO: 34, and a light chain variable region (LCVR) that comprises the LCDR1, LCDR2 and LCDR3 of a LCVR that comprises the amino acid sequence set forth in SEQ ID NO: 42;
(4) a heavy chain variable region (HCVR) that comprises the HCDR1, HCDR2 and HCDR3 of a HCVR that comprises the amino acid sequence set forth in SEQ ID NO: 50, and a light chain variable region (LCVR) that comprises the LCDR1, LCDR2 and LCDR3 of a LCVR that comprises the amino acid sequence set forth in SEQ ID NO: 58;
(5) a heavy chain variable region (HCVR) that comprises the HCDR1, HCDR2 and HCDR3 of a HCVR that comprises the amino acid sequence set forth in SEQ ID NO: 66, and a light chain variable region (LCVR) that comprises the LCDR1, LCDR2 and LCDR3 of a LCVR that comprises the amino acid sequence set forth in SEQ ID NO: 74;
(6) a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90;
(7) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 106;
(8) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 114;
(9) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 106;
(10) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 130;
(11) a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 106;
(12) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 106;
(13) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130;
(14) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 114;
(15) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 130;
(16) a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 130;
(17) a heavy chain variable region (HCVR) that comprises the HCDR1, HCDR2 and HCDR3 of a HCVR that comprises the amino acid sequence set forth in SEQ ID NO: 154, and a light chain variable region (LCVR) that comprises the LCDR1, LCDR2 and LCDR3 of a LCVR that comprises the amino acid sequence set forth in SEQ ID NO: 162;
(18) a heavy chain variable region (HCVR) that comprises the HCDR1, HCDR2 and HCDR3 of a HCVR that comprises the amino acid sequence set forth in SEQ ID NO: 170, and a light chain variable region (LCVR) that comprises the LCDR1, LCDR2 and LCDR3 of a LCVR that comprises the amino acid sequence set forth in SEQ ID NO: 178;
(19) a heavy chain variable region (HCVR) that comprises the HCDR1, HCDR2 and HCDR3 of a HCVR that comprises the amino acid sequence set forth in SEQ ID NO: 186, and a light chain variable region (LCVR) that comprises the LCDR1, LCDR2 and LCDR3 of a LCVR that comprises the amino acid sequence set forth in SEQ ID NO: 194;
(20) a heavy chain variable region (HCVR) that comprises the HCDR1, HCDR2 and HCDR3 of a HCVR that comprises the amino acid sequence set forth in SEQ ID NO: 202, and a light chain variable region (LCVR) that comprises the LCDR1, LCDR2 and LCDR3 of a LCVR that comprises the amino acid sequence set forth in SEQ ID NO: 210;
(21) a heavy chain variable region (HCVR) that comprises the HCDR1, HCDR2 and HCDR3 of a HCVR that comprises the amino acid sequence set forth in SEQ ID NO: 218, and a light chain variable region (LCVR) that comprises the LCDR1, LCDR2 and LCDR3 of a LCVR that comprises the amino acid sequence set forth in SEQ ID NO: 226;
(22) a heavy chain variable region (HCVR) that comprises the HCDR1, HCDR2 and HCDR3 of a HCVR that comprises the amino acid sequence set forth in SEQ ID NO: 234, and a light chain variable region (LCVR) that comprises the LCDR1, LCDR2 and LCDR3 of a LCVR that comprises the amino acid sequence set forth in SEQ ID NO: 242;
(23) a heavy chain variable region (HCVR) that comprises the HCDR1, HCDR2 and HCDR3 of a HCVR that comprises the amino acid sequence set forth in SEQ ID NO: 250, and a light chain variable region (LCVR) that comprises the LCDR1, LCDR2 and LCDR3 of a LCVR that comprises the amino acid sequence set forth in SEQ ID NO: 258;
(24) a heavy chain variable region (HCVR) that comprises the HCDR1, HCDR2 and HCDR3 of a HCVR that comprises the amino acid sequence set forth in SEQ ID NO: 266, and a light chain variable region (LCVR) that comprises the LCDR1, LCDR2 and LCDR3 of a LCVR that comprises the amino acid sequence set forth in SEQ ID NO: 258;
(25) a heavy chain variable region (HCVR) that comprises the HCDR1, HCDR2 and HCDR3 of a HCVR that comprises the amino acid sequence set forth in SEQ ID NO: 274, and a light chain variable region (LCVR) that comprises the LCDR1, LCDR2 and LCDR3 of a LCVR that comprises the amino acid sequence set forth in SEQ ID NO: 282;
(26) a heavy chain variable region (HCVR) that comprises the HCDR1, HCDR2 and HCDR3 of a HCVR that comprises the amino acid sequence set forth in SEQ ID NO: 290, and a light chain variable region (LCVR) that comprises the LCDR1, LCDR2 and LCDR3 of a LCVR that comprises the amino acid sequence set forth in SEQ ID NO: 298;
(27) a heavy chain variable region (HCVR) that comprises the HCDR1, HCDR2 and HCDR3 of a HCVR that comprises the amino acid sequence set forth in SEQ ID NO: 306, and a light chain variable region (LCVR) that comprises the LCDR1, LCDR2 and LCDR3 of a LCVR that comprises the amino acid sequence set forth in SEQ ID NO: 314;
(28) a heavy chain variable region (HCVR) that comprises the HCDR1, HCDR2 and HCDR3 of a HCVR that comprises the amino acid sequence set forth in SEQ ID NO: 322, and a light chain variable region (LCVR) that comprises the LCDR1, LCDR2 and LCDR3 of a LCVR that comprises the amino acid sequence set forth in SEQ ID NO: 330; and/or
(29) a heavy chain variable region (HCVR) that comprises the HCDR1, HCDR2 and HCDR3 of a HCVR that comprises the amino acid sequence set forth in SEQ ID NO: 338, and a light chain variable region (LCVR) that comprises the LCDR1, LCDR2 and LCDR3 of a LCVR that comprises the amino acid sequence set forth in SEQ ID NO: 346.

31. The co-formulation of claim 1, wherein the antibody or antigen-binding fragment thereof that binds specifically to C5 (anti-C5) comprises:
(i) a heavy chain variable region comprising an HCDR1 that comprises the amino acid sequence set forth in SEQ ID NO: 4, an HCDR2 that comprises the amino acid sequence set forth in SEQ ID NO: 6, and an HCDR3 that comprises the amino acid sequence set forth in SEQ ID NO: 8, and a light chain variable region comprising an LCDR1 that comprises the amino acid sequence set forth in SEQ ID NO: 12, an LCDR2 that comprises the amino acid sequence set forth in SEQ ID NO: 14, and an LCDR3 that comprises the amino acid sequence set forth in SEQ ID NO: 16;
(ii) a heavy chain variable region comprising an HCDR1 that comprises the amino acid sequence set forth in SEQ ID NO: 20, an HCDR2 that comprises the amino acid sequence set forth in SEQ ID NO: 22, and an HCDR3 that comprises the amino acid sequence set forth in SEQ ID NO: 24, and a light chain variable region comprising an LCDR1 that comprises the amino acid sequence set forth in SEQ ID NO: 28, an LCDR2 that comprises the amino acid sequence set forth in SEQ ID NO: 30, and an LCDR3 that comprises the amino acid sequence set forth in SEQ ID NO: 32;
(iii) a heavy chain variable region comprising an HCDR1 that comprises the amino acid sequence set forth in SEQ ID NO: 36, an HCDR2 that comprises the amino acid sequence set forth in SEQ ID NO: 38, and an HCDR3 that comprises the amino acid sequence set forth in SEQ ID NO: 40, and a light chain variable region comprising an LCDR1 that comprises the amino acid sequence set forth in SEQ ID NO: 44, an LCDR2 that comprises the amino acid sequence set forth in SEQ ID NO: 46, and an LCDR3 that comprises the amino acid sequence set forth in SEQ ID NO: 48;
(iv) a heavy chain variable region comprising an HCDR1 that comprises the amino acid sequence set forth in SEQ ID NO: 52, an HCDR2 that comprises the amino acid sequence set forth in SEQ ID NO: 54, and an HCDR3 that comprises the amino acid sequence set forth in SEQ ID NO: 56, and a light chain variable region comprising an LCDR1 that comprises the amino acid sequence set forth in SEQ ID NO: 60, an LCDR2 that comprises the amino acid sequence set forth in SEQ ID NO: 62, and an LCDR3 that comprises the amino acid sequence set forth in SEQ ID NO: 64;
(v) a heavy chain variable region comprising an HCDR1 that comprises the amino acid sequence set forth in SEQ ID NO: 68, an HCDR2 that comprises the amino acid sequence set forth in SEQ ID NO: 70, and an HCDR3 that comprises the amino acid sequence set forth in SEQ ID NO: 72, and a light chain variable region comprising an LCDR1 that comprises the amino acid sequence set forth in SEQ ID NO: 76, an LCDR2 that comprises the amino acid sequence set forth in SEQ ID NO: 78, and an LCDR3 that comprises the amino acid sequence set forth in SEQ ID NO: 80;
(vi) a heavy chain variable region comprising an HCDR1 that comprises the amino acid sequence set forth in SEQ ID NO: 84, an HCDR2 that comprises the amino acid sequence set forth in SEQ ID NO: 86, and an HCDR3 that comprises the amino acid sequence set forth in SEQ ID NO: 88, and a light chain variable region comprising an LCDR1 that comprises the amino acid sequence set forth in SEQ ID NO: 92, an LCDR2 that comprises the amino acid sequence set forth in SEQ ID NO: 94, and an LCDR3 that comprises the amino acid sequence set forth in SEQ ID NO: 96;
(vii) a heavy chain variable region comprising an HCDR1 that comprises the amino acid sequence set forth in SEQ ID NO: 100, an HCDR2 that comprises the amino acid sequence set forth in SEQ ID NO: 102, and an HCDR3 that comprises the amino acid sequence set forth in SEQ ID NO: 104, and a light chain variable region comprising an LCDR1 that comprises the amino acid sequence set forth in SEQ ID NO: 108, an LCDR2 that comprises the amino acid sequence set forth in SEQ ID NO: 110, and an LCDR3 that comprises the amino acid sequence set forth in SEQ ID NO: 112;
(viii) a heavy chain variable region comprising an HCDR1 that comprises the amino acid sequence set forth in SEQ ID NO: 100, an HCDR2 that comprises the amino acid sequence set forth in SEQ ID NO: 102, and an HCDR3 that comprises the amino acid sequence set forth in SEQ ID NO: 104, and a light chain variable region comprising an LCDR1 that comprises the amino acid sequence set forth in SEQ ID NO: 116, an LCDR2 that comprises the amino acid sequence set forth in SEQ ID NO: 118, and an LCDR3 that comprises the amino acid sequence set forth in SEQ ID NO: 120;
(ix) a heavy chain variable region comprising an HCDR1 that comprises the amino acid sequence set forth in SEQ ID NO: 124, an HCDR2 that comprises the amino acid sequence set forth in SEQ ID NO: 126, and an HCDR3 that comprises the amino acid sequence set forth in SEQ ID NO: 128, and a light chain variable region comprising an LCDR1 that comprises the amino acid sequence set forth in SEQ ID NO: 108, an LCDR2 that comprises the amino acid sequence set forth in SEQ ID NO: 110, and an LCDR3 that comprises the amino acid sequence set forth in SEQ ID NO: 112;
(x) a heavy chain variable region comprising an HCDR1 that comprises the amino acid sequence set forth in SEQ ID NO: 100, an HCDR2 that comprises the amino acid sequence set forth in SEQ ID NO: 102, and an HCDR3 that comprises the amino acid sequence set forth in SEQ ID NO: 104, and a light chain variable region comprising an LCDR1 that comprises the amino acid sequence set forth in SEQ ID NO: 132, an LCDR2 that comprises the amino acid sequence set forth in SEQ ID NO: 134, and an LCDR3 that comprises the amino acid sequence set forth in SEQ ID NO: 136;
(xi) a heavy chain variable region comprising an HCDR1 that comprises the amino acid sequence set forth in SEQ ID NO: 140, an HCDR2 that comprises the amino acid sequence set forth in SEQ ID NO: 142, and an HCDR3 that comprises the amino acid sequence set forth in SEQ ID NO: 144, and a light chain variable region comprising an LCDR1 that comprises the amino acid sequence set forth in SEQ ID NO: 108, an LCDR2 that comprises the amino acid sequence set forth in SEQ ID NO: 110, and an LCDR3 that comprises the amino acid sequence set forth in SEQ ID NO: 112;
(xii) a heavy chain variable region comprising an HCDR1 that comprises the amino acid sequence set forth in SEQ ID NO: 148, an HCDR2 that comprises the amino acid sequence set forth in SEQ ID NO: 150, and an HCDR3 that comprises the amino acid sequence set forth in SEQ ID NO: 152, and a light chain variable region comprising an LCDR1 that comprises the amino acid sequence set forth in SEQ ID NO: 108, an LCDR2 that comprises the amino acid sequence set forth in SEQ ID NO: 110, and an LCDR3 that comprises the amino acid sequence set forth in SEQ ID NO: 112;
(xiii) a heavy chain variable region comprising an HCDR1 that comprises the amino acid sequence set forth in SEQ ID NO: 124, an HCDR2 that comprises the amino acid sequence set forth in SEQ ID NO: 126, and an HCDR3 that comprises the amino acid sequence set forth in SEQ ID NO: 128, and a light chain variable region comprising an LCDR1 that comprises the amino acid sequence set forth in SEQ ID NO: 132, an LCDR2 that comprises the amino acid sequence set forth in SEQ ID NO: 134, and an LCDR3 that comprises the amino acid sequence set forth in SEQ ID NO: 136;
(xiv) a heavy chain variable region comprising an HCDR1 that comprises the amino acid sequence set forth in SEQ ID NO: 148, an HCDR2 that comprises the amino acid sequence set forth in SEQ ID NO: 150, and an HCDR3 that comprises the amino acid sequence set forth in SEQ ID NO: 152, and a light chain variable region comprising an LCDR1 that comprises the amino acid sequence set forth in SEQ ID NO: 116, an LCDR2 that comprises the amino acid sequence set forth in SEQ ID NO: 118, and an LCDR3 that comprises the amino acid sequence set forth in SEQ ID NO: 120;
(xv) a heavy chain variable region comprising an HCDR1 that comprises the amino acid sequence set forth in SEQ ID NO: 148, an HCDR2 that comprises the amino acid sequence set forth in SEQ ID NO: 150, and an HCDR3 that comprises the amino acid sequence set forth in SEQ ID NO: 152, and a light chain variable region comprising an LCDR1 that comprises the amino acid sequence set forth in SEQ ID NO: 132, an LCDR2 that comprises the amino acid sequence set forth in SEQ ID NO: 134, and an LCDR3 that comprises the amino acid sequence set forth in SEQ ID NO: 136;
(xvi) a heavy chain variable region comprising an HCDR1 that comprises the amino acid sequence set forth in SEQ ID NO: 140, an HCDR2 that comprises the amino acid sequence set forth in SEQ ID NO: 142, and an HCDR3 that comprises the amino acid sequence set forth in SEQ ID NO: 144, and a light chain variable region comprising an LCDR1 that comprises the amino acid sequence set forth in SEQ ID NO: 132, an LCDR2 that comprises the amino acid sequence set forth in SEQ ID NO: 134, and an LCDR3 that comprises the amino acid sequence set forth in SEQ ID NO: 136;
(xvii) a heavy chain variable region comprising an HCDR1 that comprises the amino acid sequence set forth in SEQ ID NO: 156, an HCDR2 that comprises the amino acid sequence set forth in SEQ ID NO: 158, and an HCDR3 that comprises the amino acid sequence set forth in SEQ ID NO: 160, and a light chain variable region comprising an LCDR1 that comprises the amino acid sequence set forth in SEQ ID NO: 164, an LCDR2 that comprises the amino acid sequence set forth in SEQ ID NO: 166, and an LCDR3 that comprises the amino acid sequence set forth in SEQ ID NO: 168;
(xviii) a heavy chain variable region comprising an HCDR1 that comprises the amino acid sequence set forth in SEQ ID NO: 172, an HCDR2 that comprises the amino acid sequence set forth in SEQ ID NO: 174, and an HCDR3 that comprises the amino acid sequence set forth in SEQ ID NO: 176, and a light chain variable region comprising an LCDR1 that comprises the amino acid sequence set forth in SEQ ID NO: 180, an LCDR2 that comprises the amino acid sequence set forth in SEQ ID NO: 182, and an LCDR3 that comprises the amino acid sequence set forth in SEQ ID NO: 184;
(xix) a heavy chain variable region comprising an HCDR1 that comprises the amino acid sequence set forth in SEQ ID NO: 188, an HCDR2 that comprises the amino acid sequence set forth in SEQ ID NO: 190, and an HCDR3 that comprises the amino acid sequence set forth in SEQ ID NO: 192, and a light chain variable region comprising an LCDR1 that comprises the amino acid sequence set forth in SEQ ID NO: 196, an LCDR2 that comprises the amino acid sequence set forth in SEQ ID NO: 198, and an LCDR3 that comprises the amino acid sequence set forth in SEQ ID NO: 200;
(xx) a heavy chain variable region comprising an HCDR1 that comprises the amino acid sequence set forth in SEQ ID NO: 204, an HCDR2 that comprises the amino acid sequence set forth in SEQ ID NO: 206, and an HCDR3 that comprises the amino acid sequence set forth in SEQ ID NO: 208, and a light chain variable region comprising an LCDR1 that comprises the amino acid sequence set forth in SEQ ID NO: 212, an LCDR2 that comprises the amino acid sequence set forth in SEQ ID NO: 214, and an LCDR3 that comprises the amino acid sequence set forth in SEQ ID NO: 216;
(xxi) a heavy chain variable region comprising an HCDR1 that comprises the amino acid sequence set forth in SEQ ID NO: 220, an HCDR2 that comprises the amino acid sequence set forth in SEQ ID NO: 222, and an HCDR3 that comprises the amino acid sequence set forth in SEQ ID NO: 224, and a light chain variable region comprising an LCDR1 that comprises the amino acid sequence set forth in SEQ ID NO: 228, an LCDR2 that comprises the amino acid sequence set forth in SEQ ID NO: 230, and an LCDR3 that comprises the amino acid sequence set forth in SEQ ID NO: 232;
(xxii) a heavy chain variable region comprising an HCDR1 that comprises the amino acid sequence set forth in SEQ ID NO: 236, an HCDR2 that comprises the amino acid sequence set forth in SEQ ID NO: 238, and an HCDR3 that comprises the amino acid sequence set forth in SEQ ID NO: 240, and a light chain variable region comprising an LCDR1 that comprises the amino acid sequence set forth in SEQ ID NO: 244, an LCDR2 that comprises the amino acid sequence set forth in SEQ ID NO: 246, and an LCDR3 that comprises the amino acid sequence set forth in SEQ ID NO: 248;
(xxiii) a heavy chain variable region comprising an HCDR1 that comprises the amino acid sequence set forth in SEQ ID NO: 252, an HCDR2 that comprises the amino acid sequence set forth in SEQ ID NO: 254, and an HCDR3 that comprises the amino acid sequence set forth in SEQ ID NO: 256, and a light chain variable region comprising an LCDR1 that comprises the amino acid sequence set forth in SEQ ID NO: 260, an LCDR2 that comprises the amino acid sequence set forth in SEQ ID NO: 262, and an LCDR3 that comprises the amino acid sequence set forth in SEQ ID NO: 264;
(xxiv) a heavy chain variable region comprising an HCDR1 that comprises the amino acid sequence set forth in SEQ ID NO: 268, an HCDR2 that comprises the amino acid sequence set forth in SEQ ID NO: 270, and an HCDR3 that comprises the amino acid sequence set forth in SEQ ID NO: 272, and a light chain variable region comprising an LCDR1 that comprises the amino acid sequence set forth in SEQ ID NO: 260, an LCDR2 that comprises the amino acid sequence set forth in SEQ ID NO: 262, and an LCDR3 that comprises the amino acid sequence set forth in SEQ ID NO: 264;
(xxv) a heavy chain variable region comprising an HCDR1 that comprises the amino acid sequence set forth in SEQ ID NO: 276, an HCDR2 that comprises the amino acid sequence set forth in SEQ ID NO: 278, and an HCDR3 that comprises the amino acid sequence set forth in SEQ ID NO: 280, and a light chain variable region comprising an LCDR1 that comprises the amino acid sequence set forth in SEQ ID NO: 284, an LCDR2 that comprises the amino acid sequence set forth in SEQ ID NO: 286, and an LCDR3 that comprises the amino acid sequence set forth in SEQ ID NO: 288;
(xxvi) a heavy chain variable region comprising an HCDR1 that comprises the amino acid sequence set forth in SEQ ID NO: 292, an HCDR2 that comprises the amino acid sequence set forth in SEQ ID NO: 294, and an HCDR3 that comprises the amino acid sequence set forth in SEQ ID NO: 296, and a light chain variable region comprising an LCDR1 that comprises the amino acid sequence set forth in SEQ ID NO: 300, an LCDR2 that comprises the amino acid sequence set forth in SEQ ID NO: 302, and an LCDR3 that comprises the amino acid sequence set forth in SEQ ID NO: 304;
(xxvii) a heavy chain variable region comprising an HCDR1 that comprises the amino acid sequence set forth in SEQ ID NO: 308, an HCDR2 that comprises the amino acid sequence set forth in SEQ ID NO: 310, and an HCDR3 that comprises the amino acid sequence set forth in SEQ ID NO: 312, and a light chain variable region comprising an LCDR1 that comprises the amino acid sequence set forth in SEQ ID NO: 316, an LCDR2 that comprises the amino acid sequence set forth in SEQ ID NO: 318, and an LCDR3 that comprises the amino acid sequence set forth in SEQ ID NO: 320;
(xxviii) a heavy chain variable region comprising an HCDR1 that comprises the amino acid sequence set forth in SEQ ID NO: 324, an HCDR2 that comprises the amino acid sequence set forth in SEQ ID NO: 326, and an HCDR3 that comprises the amino acid sequence set forth in SEQ ID NO: 328, and a light chain variable region comprising an LCDR1 that comprises the amino acid sequence set forth in SEQ ID NO: 332, an LCDR2 that comprises the amino acid sequence set forth in SEQ ID NO: 334, and an LCDR3 that comprises the amino acid sequence set forth in SEQ ID NO: 336; or
(xxix) a heavy chain variable region comprising an HCDR1 that comprises the amino acid sequence set forth in SEQ ID NO: 340, an HCDR2 that comprises the amino acid sequence set forth in SEQ ID NO: 342, and an HCDR3 that comprises the amino acid sequence set forth in SEQ ID NO: 344, and a light chain variable region comprising an LCDR1 that comprises the amino acid sequence set forth in SEQ ID NO: 348, an LCDR2 that comprises the amino acid sequence set forth in SEQ ID NO: 350, and an LCDR3 that comprises the amino acid sequence set forth in SEQ ID NO: 352.

32. The co-formulation of claim 1, wherein the antibody or antigen-binding fragment thereof that binds specifically to C5 (anti-C5) comprises:
(1) a heavy chain variable region that comprises the amino acid sequence set forth in SEQ ID NO: 2, and a light chain variable region that comprises the amino acid sequence set forth in SEQ ID NO: 10;
(2) a heavy chain variable region that comprises the amino acid sequence set forth in SEQ ID NO: 18, and a light chain variable region that comprises the amino acid sequence set forth in SEQ ID NO: 26;
(3) a heavy chain variable region that comprises the amino acid sequence set forth in SEQ ID NO: 34, and a light chain variable region that comprises the amino acid sequence set forth in SEQ ID NO: 42;
(4) a heavy chain variable region that comprises the amino acid sequence set forth in SEQ ID NO: 50, and a light chain variable region that comprises the amino acid sequence set forth in SEQ ID NO: 58;
(5) a heavy chain variable region that comprises the amino acid sequence set forth in SEQ ID NO: 66, and a light chain variable region that comprises the amino acid sequence set forth in SEQ ID NO: 74;
(6) a heavy chain variable region that comprises the amino acid sequence set forth in SEQ ID NO: 82, and a light chain variable region that comprises the amino acid sequence set forth in SEQ ID NO: 90;
(7) a heavy chain variable region that comprises the amino acid sequence set forth in SEQ ID NO: 98, and a light chain variable region that comprises the amino acid sequence set forth in SEQ ID NO: 106;
(8) a heavy chain variable region that comprises the amino acid sequence set forth in SEQ ID NO: 98, and a light chain variable region that comprises the amino acid sequence set forth in SEQ ID NO: 114;
(9) a heavy chain variable region that comprises the amino acid sequence set forth in SEQ ID NO: 122, and a light chain variable region that comprises the amino acid sequence set forth in SEQ ID NO: 106;
(10) a heavy chain variable region that comprises the amino acid sequence set forth in SEQ ID NO: 98, and a light chain variable region that comprises the amino acid sequence set forth in SEQ ID NO: 130;
(11) a heavy chain variable region that comprises the amino acid sequence set forth in SEQ ID NO: 138, and a light chain variable region that comprises the amino acid sequence set forth in SEQ ID NO: 106;
(12) a heavy chain variable region that comprises the amino acid sequence set forth in SEQ ID NO: 146, and a light chain variable region that comprises the amino acid sequence set forth in SEQ ID NO: 106;
(13) a heavy chain variable region that comprises the amino acid sequence set forth in SEQ ID NO: 122, and a light chain variable region that comprises the amino acid sequence set forth in SEQ ID NO: 130;
(14) a heavy chain variable region that comprises the amino acid sequence set forth in SEQ ID NO: 146, and a light chain variable region that comprises the amino acid sequence set forth in SEQ ID NO: 114;
(15) a heavy chain variable region that comprises the amino acid sequence set forth in SEQ ID NO: 146, and a light chain variable region that comprises the amino acid sequence set forth in SEQ ID NO: 130;
(16) a heavy chain variable region that comprises the amino acid sequence set forth in SEQ ID NO: 138, and a light chain variable region that comprises the amino acid sequence set forth in SEQ ID NO: 130;
(17) a heavy chain variable region that comprises the amino acid sequence set forth in SEQ ID NO: 154, and a light chain variable region that comprises the amino acid sequence set forth in SEQ ID NO: 162;
(18) a heavy chain variable region that comprises the amino acid sequence set forth in SEQ ID NO: 170, a light chain variable region that comprises the amino acid sequence set forth in SEQ ID NO: 178;
(19) a heavy chain variable region that comprises the amino acid sequence set forth in SEQ ID NO: 186, and a light chain variable region that comprises the amino acid sequence set forth in SEQ ID NO: 194;
(20) a heavy chain variable region that comprises the amino acid sequence set forth in SEQ ID NO: 202, and a light chain variable region that comprises the amino acid sequence set forth in SEQ ID NO: 210;
(21) a heavy chain variable region that comprises the amino acid sequence set forth in SEQ ID NO: 218, and a light chain variable region that comprises the amino acid sequence set forth in SEQ ID NO: 226;
(22) a heavy chain variable region that comprises the amino acid sequence set forth in SEQ ID NO: 234, and a light chain variable region that comprises the amino acid sequence set forth in SEQ ID NO: 242;
(23) a heavy chain variable region that comprises the amino acid sequence set forth in SEQ ID NO: 250, and a light chain variable region that comprises the amino acid sequence set forth in SEQ ID NO: 258;
(24) a heavy chain variable region that comprises the amino acid sequence set forth in SEQ ID NO: 266, and a light chain variable region that comprises the amino acid sequence set forth in SEQ ID NO: 258;
(25) a heavy chain variable region that comprises the amino acid sequence set forth in SEQ ID NO: 274, and a light chain variable region that comprises the amino acid sequence set forth in SEQ ID NO: 282;
(26) a heavy chain variable region that comprises the amino acid sequence set forth in SEQ ID NO: 290, and a light chain variable region that comprises the amino acid sequence set forth in SEQ ID NO: 298;
(27) a heavy chain variable region that comprises the amino acid sequence set forth in SEQ ID NO: 306, and a light chain variable region that comprises the amino acid sequence set forth in SEQ ID NO: 314;
(28) a heavy chain variable region that comprises the amino acid sequence set forth in SEQ ID NO: 322, and a light chain variable region that comprises the amino acid sequence set forth in SEQ ID NO: 330; or
(29) a heavy chain variable region that comprises the amino acid sequence set forth in SEQ ID NO: 338, and a light chain variable region that comprises the amino acid sequence set forth in SEQ ID NO: 346.

33. The co-formulation of claim 1, which comprises about 90 to about 275 mg/ml; or about 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 211, 220, 242, or 274 mg/ml; or at least about 150 mg/ml, at least about 175 mg/ml, at least about 200 mg/ml, at least about 211 mg/ml, at least about 220 mg/ml, at least about 242 mg/ml or at least about 274 mg/ml of the antibody or antigen-binding fragment that specifically binds to C5.

34. The co-formulation of claim 1, wherein the C5 iRNA is a double-stranded ribonucleic acid (dsRNA) agent comprising a sense strand and an antisense strand, wherein the antisense strand comprises a region of complementarity comprising at least 17 contiguous nucleotides differing by no more than 3 nucleotides from the nucleotide sequence of 5′-UAUUAUAAAAAUAUCUUGCUUUU-3′ (SEQ ID NO: 364), and wherein the dsRNA agent comprises at least one modified nucleotide.

35. The co-formulation of claim 1, wherein the C5 iRNA is a double-stranded ribonucleic acid (dsRNA) agent comprising a sense strand and an antisense strand, wherein the sense strand comprises 5′-asasGfcAfaGfaUfAfUfuUfuuAfuAfaua-3′ (SEQ ID NO: 406) and the antisense strand comprises 5′-usAfsUfuAfuaAfaAfauaUfcUfuGfcuususudTdT-3′ (SEQ ID NO: 369), wherein
a, g, c and u are 2′-0-methyl (2′-OMe) A, G, C, and U, respectively;
Af, Gf, Cf and Uf are 2′-fluoro A, G, C and U, respectively;
dT is a deoxy-thymine nucleotide;
s is a phosphorothioate linkage; and
wherein the sense strand is conjugated at the 3′-terminus to the ligand

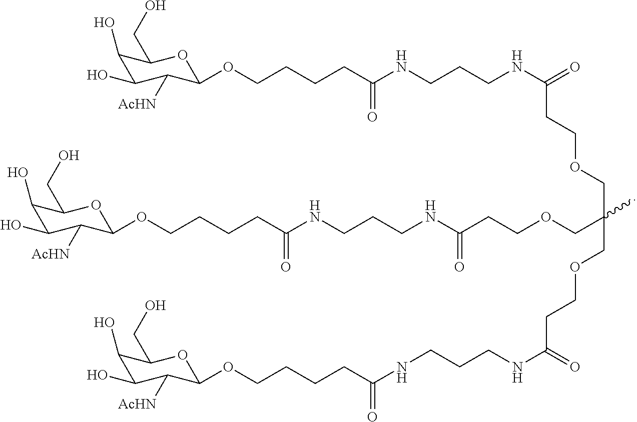 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

36. The co-formulation of claim 1, wherein the C5 iRNA is Cemdisiran or the Na⁺ salt form thereof.

37. The co-formulation of claim 1, a which comprises C5 iRNA which is Cemdisiran and one or more of Cemdisiran impurity 1, Cemdisiran impurity 2 and Cemdisiran impurity 3.

38. The co-formulation of claim 1, which comprises about 20-100, 20, 25, 30, 35, 40, 45, 50, 55, 60, 65, 70, 75, 80, 85, 90, 95, 100, 110, 115, 120, 130, 140, 150, 155, 160, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg/ml C5 iRNA.

39. The co-formulation of claim 1, which has a viscosity <30 cP at 20° C.; and/or an osmolality of 240-450 mOsm/kg.

40. The co-formulation of claim 39, which has a viscosity ≤20 cP at 20° C.

41. The co-formulation of claim 1, comprising:
a double stranded C5 iRNA; and
an anti-C5 antibody or antigen-binding fragment thereof,
a pH above or below 6.0 by at least 0.5;
a C5 iRNA,
an anti-C5 antibody or antigen-binding fragment thereof,
a buffer,
a viscosity reducer,
a stabilizer, and
a non-ionic surfactant;
a C5 iRNA,
an anti-C5 antibody or antigen-binding fragment thereof,
histidine-based buffer,
L-arginine,
a stabilizer, and
a non-ionic surfactant;
a C5 iRNA,
an anti-C5 antibody or antigen-binding fragment thereof,
histidine-based buffer,
L-arginine,
a sugar or polyol, and
a non-ionic surfactant;
Cemdisiran, Pozelimab,
histidine-based buffer,
L-arginine,
a stabilizer, and
a non-ionic surfactant,
pH about 6.5;
Cemdisiran,
Pozelimab
histidine-based buffer,
L-arginine,
sucrose, and
polysorbate 80,
pH about 6.5;
100 ±10 mg/mL C5 iRNA,
100 ±10 mg/mL anti-C5 antibody or antigen-binding fragment thereof,
50±5 mM viscosity reducer,
10±1 mM buffer,
1.0 ±0.1% stabilizer,
0.075±0.0075% non-ionic surfactant,
pH 6.5;
75±7.5 mg/mL C5 iRNA,
150±15 mg/mL anti-C5 antibody or antigen-binding fragment thereof,
75±7.5 mM viscosity reducer,
15±1.5 mM buffer,
1.5 ±0.15% stabilizer,
0.1125±0.01125% non-ionic surfactant,
pH 6.5;
50 ±5 mg/mL C5 iRNA,
100±10 mg/mL anti-C5 antibody or antigen-binding fragment thereof,
75 mM±7.5 viscosity reducer,
15±1.5 mM buffer,
1.5±0.15% stabilizer,
0.1125±0.01125% non-ionic surfactant;
pH 6.5;
50±5 mg/mL C5 iRNA,
100±10 mg/mL anti-C5 antibody or antigen-binding fragment thereof,
75±7.5 mM viscosity reducer,
35±3.5 mM buffer,
1.5±0.15% stabilizer,
0.1125±0.01125% non-ionic surfactant,
pH 6.5;
100 ±10 mg/mL C5 iRNA,
100±10 mg/mL anti-C5 antibody or antigen-binding fragment thereof,
50 ±5 mM viscosity reducer,
30 ±3 mM buffer,
1±0.1% stabilizer,
0.075±0.0075% non-ionic surfactant,
pH 6.5;
50±5 mg/mL C5 iRNA,
100±10 mg/mL anti-C5 antibody or antigen-binding fragment thereof,
90 ±9 mM viscosity reducer,
30 ±3 mM buffer,
1±0.1% stabilizer,
0.075±0.0075% non-ionic surfactant,
pH 6.5;
100 mg/mL Cemdisiran,
100 mg/mL Pozelimab,
50 mM L-arginine,
30 mM histidine-based buffer,
1% (w/v) sucrose,
0.075% (w/v) PS80,
pH 6.5;
50 mg/mL Cemdisiran,
100 mg/mL Pozelimab,
90 mM L-arginine,
30 mM histidine-based buffer,
1% (w/v) sucrose,
0.075% (w/v) PS80,
pH 6.5;
100 mg/mL Cemdisiran,
100 mg/mL Pozelimab,
50 mM L-arginine,
10 mM histidine-based buffer,
1.0% sucrose,
0.075% PS80,
pH 6.5;
75 mg/mL Cemdisiran,
150 mg/mL Pozelimab,
75 mM L-arginine,
15 mM histidine-based buffer,
1.5% sucrose,
0.1125% PS80,
pH 6.5;
50 mg/mL Cemdisiran,
100 mg/mL Pozelimab,
75 mM L-arginine,
15 mM histidine-based buffer,
1.5% sucrose,
0.1125% PS80;
pH 6.5;
50 mg/mL Cemdisiran,
100 mg/mL Pozelimab,
75 mM L-arginine,
35 mM histidine-based buffer,
1.5% sucrose,
0.1125% PS80,
pH 6.5;
100 mg/mL Cemdisiran,
100 mg/mL Pozelimab,
50 mM L-arginine,
30 mM histidine-based buffer,
1% sucrose,
0.075% PS80,
pH 6.5;
50 mg/mL Cemdisiran,
100 mg/mL Pozelimab,
90 mM L-arginine,
30 mM histidine-based buffer,
1% sucrose,
0.075% PS80,
pH 6.5;
optionally, further comprising GalNAc and/or GlcNAc;
120 mg/mL C5 iRNA,
120 mg/mL anti-C5 antibody or antigen-binding fragment,
A viscosity reducer;
15 mM histidine,
pH 6.2;
75 mg/mL C5 iRNA,
150 mg/mL anti-C5 antibody or antigen-binding fragment,
A viscosity reducer;
15 mM histidine,
pH 6.2;
120 mg/mL C5 iRNA,
120 mg/mL anti-C5 antibody or antigen-binding fragment,
15 mM histidine,
pH 6.2;
75 mg/mL C5 iRNA,
150 mg/mL anti-C5 antibody or antigen-binding fragment,
15 mM histidine,
pH 6.2;
120 mg/mL Cemdisiran,
120 mg/mL Pozelimab,
15 mM histidine,
pH 6.2;
120 mg/mL Cemdisiran,
120 mg/mL Pozelimab,
75 mM arginine,
15 mM histidine,
pH 6.2;
120 mg/mL Cemdisiran,
120 mg/mL Pozelimab,
75 mM adipate,
15 mM histidine,
pH 6.2;
120 mg/mL Cemdisiran,
120 mg/mL Pozelimab,
75 mM NaCl,
15 mM histidine,
pH 6.2;
120 mg/mL Cemdisiran,
120 mg/mL Pozelimab,
75 mM lysine,
15 mM histidine,
pH 6.2;
120 mg/mL Cemdisiran,
120 mg/mL Pozelimab,
75 mM aspartate,
15 mM histidine,
pH 6.2;
120 mg/mL Cemdisiran,
120 mg/mL Pozelimab,
75 mM proline,
15 mM histidine,
pH 6.2;
120 mg/mL Cemdisiran,
120 mg/mL Pozelimab,
50 mM histidine,
pH 6.2;
120 mg/mL Cemdisiran,
120 mg/mL Pozelimab,
50 mM caffeine,
15 mM histidine,
pH 6.2;
120 mg/mL Cemdisiran,
120 mg/mL Pozelimab,
50 mM phenylalanine,
15 mM histidine,
pH 6.2
120 mg/mL Cemdisiran,
120 mg/mL Pozelimab,
50 mM triethyl citrate,
15 mM histidine,
pH 6.2;
75 mg/mL Cemdisiran,
150 mg/mL Pozelimab,
15 mM histidine,
pH 6.2;
75 mg/mL Cemdisiran,
150 mg/mL Pozelimab,
75 mM arginine,
15 mM histidine,
pH 6.2;
75 mg/mL Cemdisiran,
150 mg/mL Pozelimab,
75 mM adipate,
15 mM histidine,
pH 6.2;
75 mg/mL Cemdisiran,
150 mg/mL Pozelimab,
75 mM NaCl,
15 mM histidine,
pH 6.2;
75 mg/mL Cemdisiran,
150 mg/mL Pozelimab,
75 mM lysine,
15 mM histidine,
pH 6.2;
or
75 mg/mL Cemdisiran,
150 mg/mL Pozelimab,
75 mM aspartate,
15 mM histidine,
pH 6.2.

42. The co-formulation of claim 1, wherein the C5 iRNA is Cemdisiran;
antibody or antigen-binding fragment is Pozelimab;
viscosity reducer is L-arginine;
buffer is a histidine-based buffer;
stabilizer is sucrose;
non-ionic surfactant is polysorbate 80; and
pH is about 6.5.

43. The co-formulation of claim 1, wherein
the C5 iRNA is conjugated to a ligand that comprises one or more terminal N-Acetylgalactosamine (GalNAc) or N-acetylglucosamine (GlcNAc) residues;
the pH is within no less than about 0.5 of about 6; and/or
the pH is about 6.5.

44. The co-formulation of claim 1, which
comprises beta-hexosaminidase;
comprises the antibody of antigen-binding fragment thereof which was expressed and isolated from a mammalian host cell that contains beta-hexosaminidase;
comprises the antibody of antigen-binding fragment thereof which was expressed and isolated from a Chinese hamster ovary cell;
comprises no more than about 1% Cemdisiran Impurity 1 relative to total Cemdisiran;
comprises no less than about 80% Cemdisiran, relative to total Cemdisiran, after 2 years storage at 2-8° C.;
has about 91% Cemdisiran before storage at t=0;
has no less than about 80% Cemdisiran after 1, 12, 2, 22 or 3 years storage at 2-8° C.;
has about 80% to about 91% Cemdisiran.
exhibits a Cemdisiran Purity (%) by dIPRP of about 90.5% at t=0; 91.1% after 1 month storage at 2-8° C.; 90.8% after 3 months storage at 2-8° C.; 90% after 6 months storage at 2-8° C.; 88.8% after 9 months storage at 2-8° C.; 88.7% after 12 months storage at 2-8° C.; 89% after 18 months storage at 2-8° C.; and/or 89.4% after 24 months storage at 2-8° C.;
exhibits a Cemdisiran Purity (%) by dIPRP of about 90.8% at t=0, 90.6% after 1 month storage at 2-8° C.; 90.5% after 3 months storage at 2-8° C.; 89.4% after 6 months storage at 2-8° C.; 88.3% after 9 months storage at 2-8° C.; 87.8% after 12 months storage at 2-8° C.; 87.8% after 18 months storage at 2-8° C.; and/or 89.4% after 24 months storage at 2-8° C.;
exhibits a Cemdisiran Single Strand Purity (%) by dIPRP of about 90.5% at t=0; 90.2% after 1 month storage at 25° C. and 60% RH; 87.8% after 3 months storage at 25° C. and 60% RH; 85.1% after 6 months storage at 25° C. and 60% RH; 90% after 0.5 months storage at 40° C. and 75% RH; 88.9% after 1 month storage at 40° C. and 75% RH; 85.8% after 3 months storage at 40° C. and 75% RH;
exhibits a Cemdisiran Purity (%) by dIPRP of about 90.8% at t=0; 88.8% after 1 month storage at 25° C. and 60% RH; 85.9% after 3 months storage at 25° C. and 60% RH; 82.3% after 6 months storage at 25° C. and 60% RH; 88.9% after 0.5 months storage at 40° C. and 75% RH; 87.3% after 1 month storage at 40° C. and 75% RH; 82.3% after 3 months storage at 40° C. and 75% RH;
exhibits a Cemdisiran purity (%) by dIPRP of about 90.9% at t=0; about 90.1% after 1 month of storage at 25° C., 60% RH; about 90.9% after 3 months of storage at 25° C., 60% RH; about 9.4% after 6 months of storage at 25° C., 60% RH; about 89.9% after 0.5 months of storage at 40° C., 75% RH; about 89.7% after 1 month of storage at 40° C., 75% RH; and/or about 89.5% after 3 months of storage at 40° C., 75% RH;
exhibits a Cemdisiran purity (%) by dIPRP of about 90.8% at t=0; about 90.2% after 1 month of storage at 25° C., 60% RH; about 90.8% after 3 months of storage at 25° C., 60% RH; about 9.3% after 6 months of storage at 25° C., 60% RH; about 89.5% after 0.5 months of storage at 40° C., 75% RH; about 89.6% after 1 month of storage at 40° C., 75% RH; and/or about 89.1% after 3 months of storage at 40° C., 75% RH;
exhibits a Cemdisiran purity (%) by dIPRP of about 90.5% at t=0; about 89.9% after 1 month of storage at 25° C., 60% RH; about 90.8% after 3 months of storage at 25° C., 60% RH; about 9.4% after 6 months of storage at 25° C., 60% RH; about 9.1% after 0.5 months of storage at 40° C., 75% RH; about 89.6% after 1 month of storage at 40° C., 75% RH; and/or about 89.9% after 3 months of storage at 40° C., 75% RH; and/or
exhibits a Cemdisiran purity (%) by dlPRP of about 91.1% at t=0; about 90% after 1 month of storage at 25° C., 60% RH; about 91% after 3 months of storage at 25° C., 60% RH; about 90.7% after 6 months of storage at 25° C., 60% RH; about 90% after 0.5 months of storage at 40° C., 75% RH; about 89.7% after 1 month of storage at 40° C., 75% RH; and/or about 89.9% after 3 months of storage at 40° C., 75% RH.

45. The co-formulation of claim 1, which comprises:
no more than about 2.1 parts per million (ppm) molar ratio of beta-hexosaminidase to antibody or antigen-binding fragment;
include no more than about 0.170 micrograms/ml beta-hexosaminidase,
include no more than about 0.04 micrograms/ml beta-hexosaminidase; and/or
about 0.04; 0.05; 0.06; 0.06; 0.0605; 0.0605; 0.0605; 0.063; 0.07; 0.07; 0.0765; 0.078; 0.08; 0.14; 0.141; 0.15; 0.1525; 0.166; or 0.17 micrograms/ml beta-hexosaminidase; or no more than any of such concentrations.

46. A method for administering the co-formulation of claim 1 to a subject comprising introducing the co-formulation into the body of the subject.

47. (canceled)

48. (canceled)

49. A method for treating or preventing a C5-associated disease or disorder in a subject in need thereof comprising administering a therapeutically effective amount of the co-formulation of claim 1 to the subject.

50. The method of claim 49, wherein the disease or disorder is: a disorder of inappropriate or undesirable complement activation; a hemodialysis complication; a lung disease or disorder; a neurological disorder; a parasitic disease; a post-ischemic reperfusion condition; a proteinuric kidney disease; a renal disorder; adult respiratory distress syndrome (ARDS); age-related macular degeneration (AMD); allergy; Alport's syndrome; Alzheimer's disease; an autoimmune disease; an immune complex disorder; an inflammatory disorder; an ocular disease; an organic dust disease; angiopathic thrombosis and protein-losing enteropathy; atherosclerosis; bronchoconstriction; bullous pemphigoid; C3 glomerulopathy; capillary leak syndrome; CHAPLE disease (CD55 deficiency with hyperactivation of complement; chemical injury due to irritant gasses and/or chemicals; chronic obstructive pulmonary disease (COPD); complement activation due to burn; complement activation due to frostbite; complement activation due to obesity; complement activation due to sepsis; Crohn's disease; diabetes; diabetic macular edema (DME); diabetic nephropathy; diabetic retinopathy; dry AMD; dyspnea; emphysema; epilepsy; fibrogenic dust diseases; geographic atrophy (GA); glomerulopathy; Goodpasture's Syndrome; Guillain-Barre Syndrome; hemolytic anemia; hemoptysis; hereditary angioedema; hyperacute allograft rejection; hypersensitivity pneumonitis; immune complex-associated inflammation; infectious disease; inflammation of an autoimmune disease; inherited CD59 deficiency; injury due to inert dusts and/or minerals; interleukin-2 induced toxicity during IL-2 therapy; lupus nephritis; membranoproliferative glomerulonephritis; membranoproliferative nephritis; mesenteric artery reperfusion after aortic reconstruction; multiple sclerosis; myasthenia gravis; myocardial infarction; neuromyelitis optica; ocular angiogenesis; Parkinson's disease; pneumonia; progressive kidney failure; psoriasis; pulmonary embolisms and infarcts; pulmonary fibrosis; pulmonary vasculitis; renal ischemia; renal ischemia-reperfusion injury; rheumatoid arthritis; schizophrenia; SLE nephritis; smoke injury; stroke; systemic inflammatory response in post-pump syndrome due to cardiopulmonary bypass or renal bypass; systemic lupus erythematosus (SLE); thermal injury; traumatic brain injury; uveitis; vasculitis; wet AMD; paroxysmal nocturnal hemoglobinuria (PNH); and/or xenograft rejection.

51. The method of claim 46, wherein the subject is administered one or more further therapeutic agents.

52. (canceled)

53. (canceled)

54. A method for increasing the stability of RNA, or for reducing beta-hexosaminidase activity, in a composition, comprising the RNA which is conjugated to a ligand that comprises one or more terminal N-Acetylgalactosamine (GalNAc) residues and/or N-acetylglucosamine (GlcNAc) residues; and beta-hexosaminidase comprising (i) adding GalNAc and/or GlcNAc to the composition and/or (ii) increasing or decreasing the pH of the composition from about 6.

55. The method of claim 54, wherein the composition comprises
the RNA, which is a C5 iRNA;
an anti-C5 antibody or antigen-binding fragment thereof that was expressed and isolated from a mammalian host cell that comprises the beta-hexosaminidase;
and, optionally,
a buffer;
a viscosity reducer;
a stabilizer; and
a non-ionic surfactant.

56. The method of claim 54, wherein the composition comprises an antibody that was expressed in a Chinese hamster ovary (CHO) cell.

57. The method of claim 54, wherein the RNA is a double stranded RNA, optionally comprising an overhang of 1 or 2 nucleotides on one or both ends.

58. The method of claim 54, wherein the RNA was chemically synthesized.

59. A method for making a co-formulation of claim 1, comprising combining the RNAi and the anti-C5 antibody or antigen-binding fragment, and (i) adding GalNAc to the co-formulation and/or (ii) adjusting the pH of the co-formulation to about or below about 6.

60. (canceled)

61. A method for administering to a subject an antibody or antigen-binding fragment thereof that binds specifically to C5 (anti-C5) in combination with a C5 iRNA comprising introducing the antibody or fragment and the iRNA into the body of the subject.

62. (canceled)

63. A method for treating or preventing a C5-associated disease or disorder in a subject in need thereof comprising administering to the subject a therapeutically effective amount of an antibody or antigen-binding fragment thereof that binds specifically to C5 in combination with a C5 iRNA which are in a single co-formulation or are in separate formulations.

64. The method of claim 61, further comprising administering one or more initial intravenous or subcutaneous loading doses of the antibody or antigen-binding fragment and/or the iRNA.

65. The method of claim 61, comprising administering one or more doses of
(1) about 400 mg of the anti-C5 antibody or antigen-binding fragment; and
(2) about 200 mg of the C5 iRNA.

66. The method of claim 61, wherein:
about 400 mg of the anti-C5 antibody or antigen-binding fragment is administered about every 2, 3 or 4 weeks (±3 days); and
about 200 mg of the C5 iRNA is administered about every 4 weeks (±3 days).

67. The method of claim 61, wherein the subject is administered:
(i) about 400 mg of the anti-C5 antibody or antigen-binding fragment subcutaneously about every 2 weeks (±3, 4, 5, 6 or 7 days), and about 200 mg of the C5 iRNA subcutaneously about every 4 weeks (±3, 4, 5, 6 or 7 days);
(ii) about 400 mg of the anti-C5 antibody or antigen-binding fragment subcutaneously about every 4 weeks (±3, 4, 5, 6 or 7 days), and about 200 mg of the C5 iRNA subcutaneously about every 4 weeks (±3, 4, 5, 6 or 7 days);
(iii) an intravenous loading dose of anti-C5 antibody or antigen-binding fragment, about 400 mg of the anti-C5 antibody or antigen-binding fragment subcutaneously and about 200 mg of the C5 iRNA subcutaneously; and then, about every 4 weeks (+3, 4, 5, 6 or 7 days) thereafter, about 400 mg of the anti-C5 antibody or antigen-binding fragment subcutaneously and about 200 mg of the C5 iRNA subcutaneously;
(iv) an intravenous loading dose of about 30 or 60 mg/kg anti-C5 antibody or antigen-binding fragment, about 400 mg of the anti-C5 antibody or antigen-binding fragment subcutaneously and about 200 mg of the C5 iRNA subcutaneously; and then, about every 4 weeks (±3, 4, 5, 6 or 7 days) thereafter, about 400 mg of the anti-C5 antibody or antigen-binding fragment subcutaneously and about 200 mg of the C5 iRNA subcutaneously;
(v) an intravenous loading dose of about 30 or 60 mg/kg anti-C5 antibody or antigen-binding fragment followed by one or more weekly subcutaneous doses of about 800 mg anti-C5 antibody or antigen-binding fragment, then, after an optional 1 week period, about 400 mg of the anti-C5 antibody or antigen-binding fragment subcutaneously and about 200 mg of the C5 iRNA subcutaneously; and then, about every 4 weeks (±3, 4, 5, 6 or 7 days) thereafter, about 400 mg of the anti-C5 antibody or antigen-binding fragment subcutaneously and about 200 mg of the C5 iRNA subcutaneously;
(vi) (a) a dose of Eculizumab intravenously and about 200 mg C5 iRNA subcutaneously; (b) a dose of the Eculizumab up to about 14 days (+3, 4, 5, 6 or 7 days) later; and (c) about another 14 or 15 days (13, 4, 5, 6 or 7 days) later, an anti-C5 antibody or antigen-binding fragment dose of 30 or 60 mg/kg body weight intravenously, an anti-C5 antibody or antigen-binding fragment about 400 mg subcutaneously and C5 iRNA about 200 mg subcutaneously and (d) about every 4 weeks (+3, 4, 5, 6 or 7 days) thereafter, a dose of anti-C5 antibody or antigen-binding fragment about 400 mg subcutaneously and C5 iRNA about 200 mg subcutaneously; or
(vii) (a) about a 200 mg SC dose of C5 iRNA; (b) about 28 days (±3, 4, 5, 6 or 7 days) later, a 30 or 60 mg/kg IV loading dose of anti-C5 antibody or antigen-binding fragment, a 400 mg SC dose of anti-C5 antibody or antigen-binding fragment and a 200 mg SC dose of C5 iRNA; and (c) about another 29 days (±3, 4, 5, 6 or 7 days) later and about every 4 weeks (±3, 4, 5, 6 or 7 days) thereafter, about a 400 mg SC dose of anti-C5 antibody or antigen-binding fragment and about a 200 mg SC dose of C5 iRNA; or
(viii) (a) about 4 weeks (±3, 4, 5, 6 or 7 days) after an administration of Ravulizumab, a 200 mg SC dose of C5 iRNA; (b) about another 28 days (±3, 4, 5, 6 or 7 days) later, a 30 or 60 mg/kg IV loading dose of anti-C5 antibody or antigen-binding fragment, a 400 mg SC dose of anti-C5 antibody or antigen-binding fragment and a 200 mg SC dose of C5 iRNA; and (c) about another 29 days (+3, 4, 5, 6 or 7 days) later and about every 4 weeks (±3, 4, 5, 6 or 7 days) thereafter, a 400 mg SC dose of anti-C5 antibody or antigen-binding fragment and a 200 mg SC dose of C5 iRNA.

68. The method of claim 61, wherein:
the anti-C5 antibody or antigen-binding fragment and C5 iRNA are administered about every 4 weeks (±3, 4, 5, 6 or 7 days) subcutaneously in a single injection of a co-formulation that comprises the anti-C5 antibody or antigen-binding fragment and C5 iRNA; and
about every 4 weeks (+3, 4, 5, 6 or 7 days) a further injection of the anti-C5 antibody or antigen-binding fragment is administered subcutaneously.

69. The method of claim 61, wherein:
the anti-C5 antibody or antigen-binding fragment and C5 iRNA are administered about every 4 weeks (±3, 4, 5, 6 or 7 days) subcutaneously in separate injections of separate formulations wherein one comprises the anti-C5 antibody or antigen-binding fragment and the other comprises the C5 iRNA; and
about every 4 weeks (+3, 4, 5, 6 or 7 days) a further injection of the anti-C5 antibody or antigen-binding fragment is administered subcutaneously.

70. The method of claim 61, wherein:
the anti-C5 antibody or antigen-binding fragment and C5 iRNA are administered about every 4 weeks (±3, 4, 5, 6 or 7 days) subcutaneously in a single injection of a co-formulation that comprises the anti-C5 antibody or antigen-binding fragment and C5 iRNA; and
about every 2 weeks (+3, 4, 5, 6 or 7 days) a further injection of the anti-C5 antibody or antigen-binding fragment is administered subcutaneously.

71. The method of claim 61, wherein:
the anti-C5 antibody or antigen-binding fragment and C5 iRNA are administered about every 4 weeks (±3, 4, 5, 6 or 7 days) subcutaneously in separate injections of separate formulations wherein one comprises the anti-C5 antibody or antigen-binding fragment and the other comprises the C5 iRNA; and
about every 2 weeks (+3, 4, 5, 6 or 7 days) a further injection of the anti-C5 antibody or antigen-binding fragment is administered subcutaneously.

72. The method of claim 61, wherein the subject is complement inhibitor naïve or has previously received Pozelimab monotherapy, Ravulizumab and/or Eculizumab therapy.

73. The method of claim 72 wherein the administration of Ravulizumab is intravenous or subcutaneous.

74. The method of claim 72 wherein the administration of Eculizumab is about 900 mg intravenously.

75. (canceled)

76. (canceled)

77. The method of claim 61, for treating or preventing a C5-associated disease or disorder in a subject in need thereof comprising administering to the subject a therapeutically effective amount of an anti-C5 antibody or antigen-binding fragment thereof and C5 iRNA, wherein the subject has previously received Eculizumab, wherein the method comprises administering to the subject:
(i) a dose of Eculizumab intravenously and 200 mg C5 iRNA subcutaneously;
(ii) a dose of the Eculizumab up to about 14 days (±3, 4, 5, 6 or 7 days) later (about day 15);
(iii) about 14 or 15 days (13, 4, 5, 6 or 7 days) later (about day 29), the anti-C5 antibody or antigen-binding fragment at a dose of about 30 or 60 mg/kg body weight intravenously, about 400 mg of the anti-C5 antibody or antigen-binding fragment subcutaneously and about 200 mg of the C5 iRNA subcutaneously; and then
(iv) starting about 28 days (+3, 4, 5, 6 or 7 days) later (about day 57) and about every about 28 days (±3, 4, 5, 6 or 7 days) thereafter, about 400 mg of the anti-C5 antibody or antigen-binding fragment subcutaneously and about 200 mg of the C5 iRNA subcutaneously.

78. The method of claim 61, for treating or preventing a C5-associated disease or disorder in a subject in need thereof comprising administering to the subject a therapeutically effective amount of an anti-C5 antibody or antigen-binding fragment thereof and a C5 iRNA, wherein the subject has previously received Ravulizumab, wherein the method comprises administering to the subject:
(i) about 28 days (±3, 4, 5, 6 or 7 days) after the last administration of Ravulizumab, about a 200 mg SC dose of C5 iRNA;
(ii) about 28 days (±3, 4, 5, 6 or 7 days) later (about day 29), about a 30 or 60 mg/kg IV dose of anti-C5 antibody or antigen-binding fragment, about a 400 mg SC dose of anti-C5 antibody or antigen-binding fragment and about a 200 mg SC dose of C5 iRNA; and then
(iii) starting about 28 days (+3, 4, 5, 6 or 7 days) later (about day 57) and about every about 28 days (13, 4, 5, 6 or 7 days) thereafter, about a 400 mg SC dose of anti-C5 antibody or antigen-binding fragment and about a 200 mg SC dose of C5 iRNA.

79. The method of claim 61, for treating or preventing a C5-associated disease or disorder in a subject in need thereof comprising administering to the subject a therapeutically effective amount of an anti-C5 antibody or antigen-binding fragment thereof and a C5 iRNA, wherein the subject has not previously received complement inhibitor treatment or not received complement inhibitor treatment recently, wherein the method comprises administering to the subject:
(i) on about day 1, an intravenous dose of about 30 or 60 mg/kg anti-C5 antibody or antigen-binding fragment, about a 400 mg subcutaneous (SC) dose of the antibody or fragment, and about a 200 mg SC dose of the C5 iRNA; and
(ii) starting about 28 days later (±3, 4, 5, 6 or 7 days) and about every 4 weeks (±3, 4, 5, 6 or 7 days) thereafter, about 400 mg SC of the anti-C5 antibody or antigen-binding fragment and about 200 mg SC of the C5 iRNA.

80. The method of claim 61, for treating or preventing a C5-associated disease or disorder in a subject in need thereof comprising administering to the subject a therapeutically effective amount of an anti-C5 antibody or antigen-binding fragment thereof and a C5 iRNA, wherein the subject has previously received anti-C5 antibody or antigen-binding fragment monotherapy, wherein the method comprises administering to the subject:
(i) starting about 7 to 8 (+3 days) days after the last dose of anti-C5 antibody or antigen-binding fragment monotherapy or when the next dose of the monotherapy is due and about every 4 weeks (±3, 4, 5, 6 or 7 days) thereafter, about a 400 mg SC dose of the anti-C5 antibody or antigen-binding fragment and about a 200 mg SC dose of the C5 iRNA; or
(ii) starting about 7 to 8 (±3 days) days after the last dose of anti-C5 antibody or antigen-binding fragment monotherapy or when the next dose of the monotherapy is due: about a 400 mg SC dose of the anti-C5 antibody or antigen-binding fragment and another the dose about every 2 weeks (±3, 4, 5, 6 or 7 days) thereafter; and about a 200 mg SC dose of the C5 iRNA and another the dose about every 4 weeks (±3, 4, 5, 6 or 7 days) thereafter.

81. A method for treating or preventing a C5-associated disease or disorder in a subject in need thereof comprising administering to the subject a therapeutically effective amount of an anti-C5 antibody or antigen-binding fragment thereof in combination with a C5 iRNA, wherein the subject has received one or more doses of a non-competing anti-C5 antibody or antigen-binding fragment:
(1) a dose of C5 iRNA after a dose of N/C Ab, but before the first dose of Pozelimab;
or
(1) a dose of C5 iRNA after a dose of N/C Ab, but before the first dose of Pozelimab; then
(2) an intravenous loading dose of Pozelimab which is the first dose of Pozelimab;
or
(1) an intravenous loading dose of Pozelimab along with a dose of Pozelimab 400 mg SC Q4W and Cemdisiran 200 mg SC Q4W; then
(2) starting about 4 weeks thereafter (and continuing every 4 weeks thereafter), Pozelimab 400 mg SC Q4W and Cemdisiran 200 mg SC Q4W;
or
(1) a dose of C5 iRNA after a dose of the N/C Ab has been administered and about 4 weeks before an intravenous loading dose of Pozelimab along with a dose of Pozelimab 400 mg SC Q4W and Cemdisiran 200 mg SC Q4W; then
(2) starting about 4 weeks thereafter (and continuing every 4 weeks thereafter), Pozelimab 400 mg SC Q4W and Cemdisiran 200 mg SC Q4W;
or
(1) a dose of C5 iRNA and the non-competing antibody or fragment (N/C Ab) on the day the dose of N/C Ab is due;
(2) the next dose of N/C Ab on the day such dose is due;
(3) after about 1-2 half-lives of the N/C Ab or when the next dose of N/C Ab would be due, a Pozelimab 30 or 60 mg/kg IV loading dose, and Pozelimab 400 mg SC and Cemdisiran 200 mg SC;
(4) starting 4 weeks thereafter (and continuing every 4 weeks thereafter), Pozelimab 400 mg SC Q4W and Cemdisiran 200 mg SC Q4W;
or
(1) following about 1-2 half-lives of the N/C Ab from the last dose thereof, or, on the day the next dose of N/C Ab is due; or, after half of the interval between doses has elapsed since the last dose of N/C Ab, a dose of C5 iRNA;
(2) following about another 1-2 half-lives of the N/C Ab, Pozelimab 30 or 60 mg/kg IV loading dose, Pozelimab 400 mg SC and Cemdisiran 200 mg SC; and
(3) starting 4 weeks thereafter (and continuing every 4 weeks thereafter), Pozelimab 400 mg SC Q4W and Cemdisiran 200 mg SC Q4W.

82. The method of claim 81 wherein:
the dose of C5 iRNA is administered 2, 3, 4, 5, 6, 7 or 8 weeks after a dose of N/C Ab, but 1, 2, 3 or 4 weeks before the first dose of Pozelimab;
the C5 iRNA is Cemdisiran;
the C5 iRNA is Cemdisiran and the dose is 200 mg SC;
the anti-C5 antibody or antigen-binding fragment thereof is Pozelimab;
the intravenous loading dose of Pozelimab is 30 or 60 mg/kg;
the non-competing anti-C5 antibody or antigen-binding fragment is Eculizumab;
the non-competing anti-C5 antibody or antigen-binding fragment is Eculizumab and doses are due every 1, 2, 3 or 4 weeks;
the non-competing anti-C5 antibody or antigen-binding fragment is Ravulizumab;
the non-competing anti-C5 antibody or antigen-binding fragment is Ravulizumab and doses are due every 4 or 8 weeks;
the half-life of the non-competing antibody is about 11 days; or
the half-life of the non-competing antibody is about 32 days.

83. The method of claim 46 wherein, during treatment, the subject achieves or achieves and maintains any one or more of:
hemoglobin stabilization;
does not receive a red blood cell transfusion;
has no decrease in hemoglobin ≥2 g/dL;
does not experience breakthrough hemolysis; CH50 levels in blood are fully suppressed relative to baseline (at 0 kIU/L) before treatment and/or during any breakthrough hemolysis event;
lack of treatment emergent adverse events;
improvement in fatigue, relative to before treatment;
>5 point improvement in FACIT-Fatigue score relative to before treatment;
improvement in physical functioning score on the European;
organization for Research and Treatment of Cancer: Quality-of-Life Questionnaire; core 30 items (EORTC QLQ-C30)) relative to before treatment;
improvement in GHS/QoL (global health status/QOL scale (GHS)), relative to before treatment;
reduction in lactate dehydrogenase (LDH) levels relative to before treatment;
achievement of LDH≤1.5× upper limit of normal (ULN) relative to before treatment
achievement and maintenance of LDH≤1.0×ULN;
a reduction in blood bilirubin levels relative to before treatment;
a reduction in reticulocyte count relative to before treatment;
a reduction in alternative pathway hemolytic activity assay (AH50) relative to before treatment;
a reduction in PNH erythrocytes and/or granulocytes relative to before treatment;
improvement in fatigue, shortness of breath, muscle weakness, headache, abdominal, pain, pain in back/legs, chest discomfort, difficulty sleeping, difficulty thinking clearly, and/or difficulty swallowing relative to before treatment;
improvement in renal function as measured by estimated glomerular filtration rate (eGFR) relative to before treatment;
reduction in blood free hemoglobin relative to before treatment;
reduction in total C5 blood levels relative to before treatment;
reduction in PNH clone size relative to before treatment; and/or
increase in haptoglobin level relative to before treatment.

84. The method of claim 61, wherein the C5-associated disease or disorder is a disorder of inappropriate or undesirable complement activation; a hemodialysis complication; a lung disease or disorder; a neurological disorder; a parasitic disease; a post-ischemic reperfusion condition; a proteinuric kidney disease; a renal disorder; adult respiratory distress syndrome (ARDS); age-related macular degeneration (AMD); allergy; Alport's syndrome; Alzheimer's disease; an autoimmune disease; an immune complex disorder; an inflammatory disorder; an ocular disease; an organic dust disease; angiopathic thrombosis and protein-losing enteropathy; atherosclerosis; bronchoconstriction; bullous pemphigoid; C3 glomerulopathy; capillary leak syndrome; CHAPLE disease (CD55 deficiency with hyperactivation of complement; chemical injury due to irritant gasses and/or chemicals; chronic obstructive pulmonary disease (COPD); complement activation due to burn; complement activation due to frostbite; complement activation due to obesity; complement activation due to sepsis; Crohn's disease; diabetes; diabetic macular edema (DME); diabetic nephropathy; diabetic retinopathy; dry AMD; dyspnea; emphysema; epilepsy; fibrogenic dust diseases; geographic atrophy (GA); glomerulopathy; Goodpasture's Syndrome; Guillain-Barre Syndrome; hemolytic anemia; hemoptysis; hereditary angioedema; hyperacute allograft rejection; hypersensitivity pneumonitis; immune complex-associated inflammation; infectious disease; inflammation of an autoimmune disease; inherited CD59 deficiency; injury due to inert dusts and/or minerals; interleukin-2 induced toxicity during IL-2 therapy; lupus nephritis; membranoproliferative glomerulonephritis; membranoproliferative nephritis; mesenteric artery reperfusion after aortic reconstruction; multiple sclerosis; myasthenia gravis; myocardial infarction; neuromyelitis optica; ocular angiogenesis; Parkinson's disease; pneumonia; progressive kidney failure; psoriasis; pulmonary embolisms and infarcts; pulmonary fibrosis; pulmonary vasculitis; renal ischemia; renal ischemia-reperfusion injury; rheumatoid arthritis; schizophrenia; SLE nephritis; smoke injury; stroke; systemic inflammatory response in post-pump syndrome due to cardiopulmonary bypass or renal bypass; systemic lupus erythematosus (SLE); thermal injury; traumatic brain injury; uveitis; vasculitis; wet AMD; paroxysmal nocturnal hemoglobinuria (PNH); and/or xenograft rejection.

85. The method of claim 46, wherein the C5 iRNA and the anti-C5 antibody or antigen-binding fragment are co-formulated into a co-formulation and both the antibody or fragment and the C5 iRNA are administered by way of a single injection of the co-formulation.

86. The method of claim 85 wherein the co-formulation has a pH of about 6.5.

87. The method of claim 46, wherein the C5 iRNA and the anti-C5 antibody or antigen-binding fragment are co-formulated into a co-formulation comprising 100 mg/ml Cemdisiran and 100 mg/ml Pozelimab; or 50 mg/ml Cemdisiran and 100 mg/ml Pozelimab.

88. The method of claim 46, wherein t co formulation comprises:
Cemdisiran;
Pozelimab that was expressed and isolated from a mammalian host cell that includes beta-hexosaminidase;
a buffer;
a viscosity reducer;
a stabilizer; and
a non-ionic surfactant;
at a pH of about 6.5.

89. The method of claim 62, wherein the subcutaneous injection is performed with a pre-filled syringe.

90. The method of claim 46, wherein the subject suffers from aplastic anemia and/or myelodysplastic syndrome.

91. The method of claim 46, wherein the subject has previously received or which further comprises administering, before, after or during the administering of 400 mg subcutaneous Pozelimab and 200 mg subcutaneous Cemdisiran, to the subject:
one or more doses of subcutaneous or intravenous Pozelimab;
one or more 400 mg subcutaneous doses of Pozelimab;
one or more doses of subcutaneous or intravenous anti-C5 antibody or antigen-binding fragment;
one or more doses of subcutaneous or intravenous Eculizumab;
one or more doses of subcutaneous or intravenous Ravulizumab;
one or more doses of subcutaneous or intravenous Cemdisiran;
one or more doses of subcutaneous or intravenous C5 iRNA;
one or more subcutaneous doses of 800 mg Pozelimab;
one or more subcutaneous doses of 800 mg anti-C5 antibody or antigen-binding fragment;
one or more intravenous doses of 30 mg/kg body weight Pozelimab;
one or more intravenous doses of 30 mg/kg body weight anti-C5 antibody or antigen-binding fragment;
one or more intravenous doses of about 60 mg/kg body weight of Pozelimab;
one or more intravenous doses of about 60 mg/kg body weight of anti-C5 antibody or antigen-binding fragment;
one or more subcutaneous doses of about 800 mg of Pozelimab;
one or more subcutaneous doses of about 800 mg of anti-C5 antibody or antigen-binding fragment;
one intravenous dose of about 60 mg/kg body weight of Pozelimab and then one or more subcutaneous doses of about 800 mg of Pozelimab;
one intravenous dose of about 60 mg/kg body weight of anti-C5 antibody or antigen-binding fragment and then one or more subcutaneous doses of about 800 mg of anti-C5 antibody or antigen-binding fragment;
one or more doses of ≥300, ≥600, ≥900 or ≥1200 mg of Eculizumab intravenously;
one or more doses of 200 mg of Cemdisiran subcutaneously;
and/or
one or more doses of 200 mg of C5 iRNA subcutaneously.

92. The method of claim 46, wherein:
intravenous administration of anti-C5 antibody or antigen-binding fragment is separated from subcutaneous administration of anti-C5 antibody or antigen-binding fragment or C5 iRNA by about 30 minutes;
subcutaneous administration of anti-C5 antibody or antigen-binding fragment and C5 iRNA is followed by an observation period of about 30 minutes, 1 hour or 2 hours; and/or
subcutaneous administration of C5 iRNA is followed by an observation period of about 30 minutes, 1 hour or 2 hours.

93. The method of claim 46 wherein, if the subject exhibits one or more of the criteria:
breakthrough hemolysis that is not due to a complement activating condition; and/or
LDH increase 2×ULN due to a complement activating condition,
then the subject receives an intensified treatment further comprising one or more 30 mg/kg IV dose of anti-C5 antibody or antigen-binding fragment.

94. The method of claim 46 wherein, if the subject exhibits one or more of the criteria:
breakthrough hemolysis that is not due to a complement activating condition; and/or
LDH increase ≥2×ULN due to a complement activating condition,
then the subject receives an intensified treatment wherein:
(1) if the subject had received a treatment regimen comprising about 400 mg of the anti-C5 antibody or antigen-binding fragment administered subcutaneously about every 4 weeks (±3, 4, 5, 6 or 7 days), and about 200 mg of the C5 iRNA administered subcutaneously about every 4 weeks (±3, 4, 5, 6 or 7 days); then
administering a single 30 mg/kg IV dose of anti-C5 antibody or antigen-binding fragment on the day of intensification and an intensified regimen of about 400 mg of the anti-C5 antibody or antigen-binding fragment administered subcutaneously about every 2 weeks (13, 4, 5, 6 or 7 days), and about 200 mg of the C5 iRNA administered subcutaneously about every 4 weeks (±3, 4, 5, 6 or 7 days) is administered starting on the day of intensification; or
(2) if the subject had received a treatment regimen comprising about 400 mg of the anti-C5 antibody or antigen-binding fragment administered subcutaneously about every 2 weeks (±3, 4, 5, 6 or 7 days), and about 200 mg of the C5 iRNA administered subcutaneously about every 4 weeks (±3, 4, 5, 6 or 7 days); then
administering a single 30 mg/kg IV dose of anti-C5 antibody or antigen-binding fragment on the day of intensification and re-initiation of the treatment regimen comprising about 400 mg of the anti-C5 antibody or antigen-binding fragment administered subcutaneously about every 2 weeks (±3, 4, 5, 6 or 7 days), and about 200 mg of the C5 iRNA administered subcutaneously about every 4 weeks (±3, 4, 5, 6 or 7 days) starting on the day of intensification.

95. The method of claim 46, wherein the anti-C5 antibody or antigen-binding fragment or Pozelimab is expressed in a mammalian host cell and the iRNA or Cemdisiran is chemically synthesized.

96. The method of claim 95 wherein the host cell is a Chinese hamster ovary cell.

97. The method of claim 46, wherein the anti-C5 antibody or antigen-binding fragment and C5 iRNA are co-formulated into a co-formulation that comprises
no more than about 2.1 parts per million (ppm) molar ratio of beta-hexosaminidase to antibody or antigen-binding fragment;
no more than about 0.170 micrograms/ml beta-hexosaminidase,
no more than about 0.04 micrograms/ml beta-hexosaminidase; or
about 0.04; 0.05; 0.06; 0.0605; 0.063; 0.07; 0.0765; 0.078; 0.08; 0.14; 0.141; 0.15; 0.1525; 0.166; or 0.17 micrograms/ml beta-hexosaminidase; or no more than any of such concentrations.

98. The method of claim 61, wherein the antibody or antigen-binding fragment thereof is
(1) a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10;
(2) a heavy chain variable region (HCVR) that comprises the HCDR1, HCDR2 and HCDR3 of a HCVR that comprises the amino acid sequence set forth in SEQ ID NO: 18, and a light chain variable region (LCVR) that comprises the LCDR1, LCDR2 and LCDR3 of a LCVR that comprises the amino acid sequence set forth in SEQ ID NO: 26;
(3) a heavy chain variable region (HCVR) that comprises the HCDR1, HCDR2 and HCDR3 of a HCVR that comprises the amino acid sequence set forth in SEQ ID NO: 34, and a light chain variable region (LCVR) that comprises the LCDR1, LCDR2 and LCDR3 of a LCVR that comprises the amino acid sequence set forth in SEQ ID NO: 42;
(4) a heavy chain variable region (HCVR) that comprises the HCDR1, HCDR2 and HCDR3 of a HCVR that comprises the amino acid sequence set forth in SEQ ID NO: 50, and a light chain variable region (LCVR) that comprises the LCDR1, LCDR2 and LCDR3 of a LCVR that comprises the amino acid sequence set forth in SEQ ID NO: 58;
(5) a heavy chain variable region (HCVR) that comprises the HCDR1, HCDR2 and HCDR3 of a HCVR that comprises the amino acid sequence set forth in SEQ ID NO: 66, and a light chain variable region (LCVR) that comprises the LCDR1, LCDR2 and LCDR3 of a LCVR that comprises the amino acid sequence set forth in SEQ ID NO: 74;
(6) a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90;
(7) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 106;
(8) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 114;
(9) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 106;
(10) a heavy chain variable region (HCVR) that comprises the HCDR1, HCDR2 and HCDR3 of a HCVR that comprises the amino acid sequence set forth in SEQ ID NO: 98, and a light chain variable region (LCVR) that comprises the LCDR1, LCDR2 and LCDR3 of a LCVR that comprises the amino acid sequence set forth in SEQ ID NO: 130;
(11) a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 106;
(12) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 106;
(13) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130;
(14) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 114;
(15) a heavy chain variable region (HCVR) that comprises the HCDR1, HCDR2 and HCDR3 of a HCVR that comprises the amino acid sequence set forth in SEQ ID NO: 146, and a light chain variable region (LCVR) that comprises the LCDR1, LCDR2 and LCDR3 of a LCVR that comprises the amino acid sequence set forth in SEQ ID NO: 130;
(16) a heavy chain variable region (HCVR) that comprises the HCDR1, HCDR2 and HCDR3 of a HCVR that comprises the amino acid sequence set forth in SEQ ID NO: 138, and a light chain variable region (LCVR) that comprises the LCDR1, LCDR2 and LCDR3 of a LCVR that comprises the amino acid sequence set forth in SEQ ID NO: 130;
(17) a heavy chain variable region (HCVR) that comprises the HCDR1, HCDR2 and HCDR3 of a HCVR that comprises the amino acid sequence set forth in SEQ ID NO: 154, and a light chain variable region (LCVR) that comprises the LCDR1, LCDR2 and LCDR3 of a LCVR that comprises the amino acid sequence set forth in SEQ ID NO: 162;
(18) a heavy chain variable region (HCVR) that comprises the HCDR1, HCDR2 and HCDR3 of a HCVR that comprises the amino acid sequence set forth in SEQ ID NO: 170, and a light chain variable region (LCVR) that comprises the LCDR1, LCDR2 and LCDR3 of a LCVR that comprises the amino acid sequence set forth in SEQ ID NO: 178;
(19) a heavy chain variable region (HCVR) that comprises the HCDR1, HCDR2 and HCDR3 of a HCVR that comprises the amino acid sequence set forth in SEQ ID NO: 186, and a light chain variable region (LCVR) that comprises the LCDR1, LCDR2 and LCDR3 of a LCVR that comprises the amino acid sequence set forth in SEQ ID NO: 194;
(20) a heavy chain variable region (HCVR) that comprises the HCDR1, HCDR2 and HCDR3 of a HCVR that comprises the amino acid sequence set forth in SEQ ID NO: 202, and a light chain variable region (LCVR) that comprises the LCDR1, LCDR2 and LCDR3 of a LCVR that comprises the amino acid sequence set forth in SEQ ID NO: 210;
(21) a heavy chain variable region (HCVR) that comprises the HCDR1, HCDR2 and HCDR3 of a HCVR that comprises the amino acid sequence set forth in SEQ ID NO: 218, and a light chain variable region (LCVR) that comprises the LCDR1, LCDR2 and LCDR3 of a LCVR that comprises the amino acid sequence set forth in SEQ ID NO: 226;
(22) a heavy chain variable region (HCVR) that comprises the HCDR1, HCDR2 and HCDR3 of a HCVR that comprises the amino acid sequence set forth in SEQ ID NO: 234, and a light chain variable region (LCVR) that comprises the LCDR1, LCDR2 and LCDR3 of a LCVR that comprises the amino acid sequence set forth in SEQ ID NO: 242;
(23) a heavy chain variable region (HCVR) that comprises the HCDR1, HCDR2 and HCDR3 of a HCVR that comprises the amino acid sequence set forth in SEQ ID NO: 250, and a light chain variable region (LCVR) that comprises the LCDR1, LCDR2 and LCDR3 of a LCVR that comprises the amino acid sequence set forth in SEQ ID NO: 258;
(24) a heavy chain variable region (HCVR) that comprises the HCDR1, HCDR2 and HCDR3 of a HCVR that comprises the amino acid sequence set forth in SEQ ID NO: 266, and a light chain variable region (LCVR) that comprises the LCDR1, LCDR2 and LCDR3 of a LCVR that comprises the amino acid sequence set forth in SEQ ID NO: 258;
(25) a heavy chain variable region (HCVR) that comprises the HCDR1, HCDR2 and HCDR3 of a HCVR that comprises the amino acid sequence set forth in SEQ ID NO: 274, and a light chain variable region (LCVR) that comprises the LCDR1, LCDR2 and LCDR3 of a LCVR that comprises the amino acid sequence set forth in SEQ ID NO: 282;
(26) a heavy chain variable region (HCVR) that comprises the HCDR1, HCDR2 and HCDR3 of a HCVR that comprises the amino acid sequence set forth in SEQ ID NO: 290, and a light chain variable region (LCVR) that comprises the LCDR1, LCDR2 and LCDR3 of a LCVR that comprises the amino acid sequence set forth in SEQ ID NO: 298;
(27) a heavy chain variable region (HCVR) that comprises the HCDR1, HCDR2 and HCDR3 of a HCVR that comprises the amino acid sequence set forth in SEQ ID NO: 306, and a light chain variable region (LCVR) that comprises the LCDR1, LCDR2 and LCDR3 of a LCVR that comprises the amino acid sequence set forth in SEQ ID NO: 314;
(28) a heavy chain variable region (HCVR) that comprises the HCDR1, HCDR2 and HCDR3 of a HCVR that comprises the amino acid sequence set forth in SEQ ID NO: 322, and a light chain variable region (LCVR) that comprises the LCDR1, LCDR2 and LCDR3 of a LCVR that comprises the amino acid sequence set forth in SEQ ID NO: 330; and/or
(29) a heavy chain variable region (HCVR) that comprises the HCDR1, HCDR2 and HCDR3 of a HCVR that comprises the amino acid sequence set forth in SEQ ID NO: 338, and a light chain variable region (LCVR) that comprises the LCDR1, LCDR2 and LCDR3 of a LCVR that comprises the amino acid sequence set forth in SEQ ID NO: 346.

99. The method of claim 46, wherein the C5 iRNA comprises an RNA strand that is complementary to an mRNA transcribed from the C5 gene sense strand DNA sequence AAGCAAGATATTTTTATAATA (nucleotides 782-802 of SEQ ID NO: 360).

100. The method of claim 46, wherein the C5 iRNA is a double-stranded ribonucleic acid (dsRNA) agent comprising a sense strand and an antisense strand, wherein the antisense strand comprises a region of complementarity comprising at least 17 contiguous nucleotides differing by no more than 3 nucleotides from the nucleotide sequence of 5′-UAUUAUAAAAAUAUCUUGCUUUU-3′ (SEQ ID NO: 364), and wherein the dsRNA agent comprises at least one modified nucleotide.

101. The method of claim 46, wherein the C5 iRNA is a double-stranded ribonucleic acid (dsRNA) agent comprising a sense strand and an antisense strand, wherein the sense strand comprises 5′-asasGfcAfaGfaUfAfUfuUfuuAfuAfaua-3′ (SEQ ID NO: 406) and the antisense strand comprises 5′-usAfsUfuAfuaAfaAfauaUfcUfuGfcuususudTdT-3′ (SEQ ID NO: 369), wherein
a, g, c and u are 2′-0-methyl (2′-OMe) A, G, C, and U, respectively;
Af, Gf, Cf and Uf are 2′-fluoro A, G, C and U, respectively;
dT is a deoxy-thymine nucleotide;
s is a phosphorothioate linkage; and
wherein the sense strand is conjugated at the 3′-terminus to the ligand

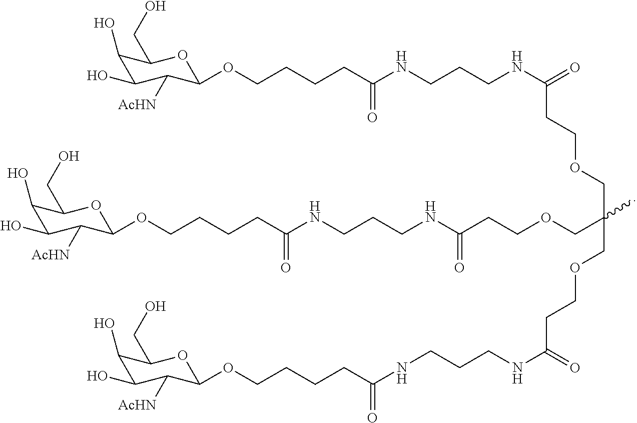 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

102. The method of claim 61, wherein the C5 iRNA and the antibody or antigen-binding fragment thereof that binds specifically to C5 are in a co-formulation.

103. The method of claim 46, wherein the C5 iRNA and the anti-C5 antibody or antigen-binding fragment thereof are in a single co-formulation which, when administered subcutaneously, is administered in 1 or 2 or more injections of said co-formulation.

104. The method of claim 46, wherein the C5 iRNA and the anti-C5 antibody or antigen-binding fragment thereof are in a single co-formulation which, when administered subcutaneously, is administered in 2 injections of said co-formulation.

105. The method of claim 46, wherein the C5 iRNA is Cemdisiran.

106. The method of claim 105, wherein the Cemdisiran is the Na⁺ salt form.

107. The method of claim 46, wherein the anti-C5 antibody or antigen-binding fragment thereof is Pozelimab.

108. The method of claim 91, wherein said 30 mg/kg or 60 mg/kg IV dose of Pozelimab or anti-C5 antibody or antigen-binding fragment thereof is an 30 mg/kg IV dose.

109. The method of claim 91, wherein said 30 mg/kg or 60 mg/kg IV dose of Pozelimab or anti-C5 antibody or antigen-binding fragment thereof is an 60 mg/kg IV dose.

110. The co-formulation of claim 1, wherein the anti-C5 antibody or antigen-binding fragment comprises a heavy chain comprising the amino acid sequence:
QVQLQESGPGLVKPSETLSLTCTVSGDSVSSSYWTWIRQPPGKGLEWIGYIYYSGSSNYN PSLKSRATISVDTSKNQFSLKLSSVTAADTAVYYCAREGNVDTTMIFDYWGQGTLVTVS SASTKGPSVFPLAPCSRSTSESTAALGCLVKDYFPEPVTVSWNSGALTSGVHTFPAVLQS SGLYSLSSVVTVPSSSLGTKTYTCNVDHKPSNTKVDKRVESKYGPPCPPCPAPEFLGGPS VFLFPPKPKDTLMISRTPEVTCVVVDVSQEDPEVQFNWYVDGVEVHNAKTKPREEQFNS TYRVVSVLTVLHQDWLNGKEYKCKVSNKGLPSSIEKTISKAKGQPREPQVYTLPPSQEE MTKNQVSLTCLVKGFYPSDIAVEWESNGQPENNYKTTPPVLDSDGSFFLYSRLTVDKSR WQEGNVFSCSVMHEALHNHYTQKSLSLSLGK; optionally, lacking the C-terminal Lysine; and a light chain comprising the amino acid sequence:
AIQMTQSPSSLSASVGDRVTITCRASQGIRNDLGWYQQKPGKA
PKLLIYAASSLQSGVPSRFAGRGSGTDFTLTISSLQPEDFATY
YCLQDFNYPWTFGQGTKVEIKRTVAAPSVFIFPPSDEQLKSGT
ASVVCLLNNFYPREAKVQWKVDNALQSGNSQESVTEQDSKDST
YSLSSTLTLSKADYEKHKVYACEVTHQGLSSPVTKSFNRGEC.
